(19) United States
(12) Patent Application Publication
(10) Pub. No.: US 2023/0312577 A1
(43) Pub. Date: Oct. 5, 2023

(54) DUAL KINASE-BROMODOMAIN INHIBITORS

(71) Applicants: MONASH UNIVERSITY, Clayton, Victoria (AU); PETER MACCALLUM CANCER INSTITUTE, East Melbourne, Victoria (AU)

(72) Inventors: Philip THOMPSON, Clayton (AU); Jake SHORTT, Clayton (AU); Ricky JOHNSTONE, East Melbourne (AU); Xiao MA, Clayton (AU); Danielle OH, Clayton (AU); Ian JENNINGS, Clayton (AU)

(21) Appl. No.: 18/000,632

(22) PCT Filed: Jun. 4, 2021
(86) PCT No.: PCT/AU2021/050562

§ 371 Date: Dec. 2, 2022

(30) Foreign Application Priority Data
Jun. 5, 2020 (AU) 2020901867

(51) Int. Cl. C07D 487/04; C07D 495/04; C07D 471/04; A61P 35/00

(52) U.S. Cl. CPC C07D 487/04; C07D 495/04; C07D 471/04; A61P 35/00

(57) ABSTRACT

Provided herein are compounds of Formula (I) that are dual inhibitors of kinases and bromo-domain proteins. The disclosure also relates to pharmaceutical compositions containing such compounds, methods for using such compounds in the treatment of cancers, particularly, the treatment of multiple myeloma cancers, and to related uses.

### FIELD

The present disclosure relates to compounds that are dual inhibitors of kinases and bromodomain proteins. The disclosure also relates to pharmaceutical compositions containing such compounds, methods for using such compounds in the treatment of cancers, particularly, the treatment of multiple myeloma cancers, and to related uses.

### BACKGROUND

Cancer is the term given to a collection of related diseases in which abnormal cells divide in an uncontrolled manner, such that they invade nearby tissues. Cancer is recognised as a leading cause of death, with the Agency for Research on Cancer estimating 18.1 million new cancer cases, and 9.6 million cancer deaths, in 2018.

Some cancers cause tumors of the haematopoietic and lymphoid tissues, producing tumours that affect the blood, bone marrow, lymph, and lymphatic system. Together, these cancers may derive from either of the two major blood cell lineages—myeloid and lymphoid cell lines—and are commonly referred to as “blood cancers”.

One example is multiple myeloma, also known as plasma cell myeloma, which is a cancer of plasma cells. This is the second most common haematological cancer which often results in multi-system disease involving bone marrow, kidneys, bones, and immune system. Despite significant advances in the treatment landscape of multiple myeloma, it still remains incurable to date.

In multiple myeloma, and indeed many other cancers, it has been found that the Myc gene is frequently altered. This manifests in Myc often being persistently over-expressed.

The Myc gene encodes a transcription factor that binds to and regulates nearly 10-15% of genes in the human genome. The Myc targets mediate fundamental biological processes necessary for cell survival and general well-being, ranging from gene expression and cell-cycle programs to cell proliferation and response and to DNA damage, thereby establish Myc as a global transcription regulator. Consequently, Myc is associated with not only cancer, but a number of physiological disorders.

There have been significant efforts into the research and development of new and efficacious oncology agents. Indeed, there have been significant advances in the chemotherapeutic treatment of cancer in recent decades. However, therapeutic application of oncology agents is often hampered by difficulties associated with their formulation and delivery, including poor pharmacokinetics properties such as rapid metabolism and/or excretion, and/or lack of targeting to the site of action. Further, a number of oncology agents are associated with severe side effects, providing a narrow therapeutic window, limiting the dosage regimen that can be used, and potentially reducing the efficacy of the treatment. Accordingly, modern cancer therapy has thus far proven only partially successful in treating and prolonging the lives of patients with many types of cancer.

Accordingly, there remains a need to develop safe and efficacious oncology agents, wherein the pharmacokinetic/pharmacodynamic properties provide for potent, selective, long-lasting, and overall controlled delivery of therapeutics, all the while resulting in fewer side-effects to the patient.

### SUMMARY

The present inventors have undertaken extensive research into the development of dual inhibitors and have surprisingly identified that compounds of Formula (I) can provide effective dual inhibitors of kinases and bromodomain proteins.

Accordingly, in one aspect there is provided a compound of Formula (I), or a pharmaceutically acceptable salt, solvate or stereoisomer thereof:

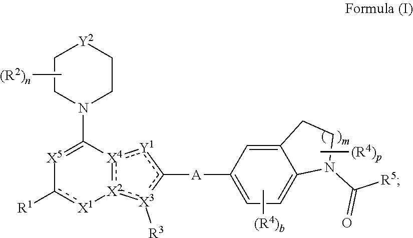

wherein X¹ is C(R) or N; X² is CH, C or N; X³ is C or N; X⁴ is C or N; X⁵ is C or N; Y¹ is CH, N or S; Y² is O, CH₂, or N(R); R is selected from the group consisting of hydrogen, halogen, and C₁₋₆alkyl; R¹, R², R³ and R⁴ are each independently selected from the group consisting of hydrogen, halogen, —C₁₋₆alkyl, —O—C₁₋₆alkyl, —C₁₋₆haloalkyl, —O—C₁₋₆haloalkyl, —C₂-6alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷); wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and wherein each —C₁₋₆alkyl is unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶; R⁵ is selected from the group consisting of —C₁₋₆alkyl, —O(R⁶), 3-10 membered carbocyclyl, and 3-10 membered heterocyclyl; m is 1, 2, or 3; n is 0, 1, 2, 3, 4, 5, 6, 7, or 8; p is 0, 1, 2, 3, 4, 5, 6, 7, or 8; bis 0, 1, 2, or 3;

A is a linker of Formula (II):

-[A¹]ᵤ₁-[Z¹]ₜ₁-[A²]ᵤ₂-[Z²]ₜ₂-[A³]ᵤ₃-[Z³]ₜ₃— Formula (II);
wherein A¹, A² and A³ are each independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ or —C(O)R⁶; Z¹, Z² and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶ or —C(O)R⁶; u¹, u² and u³ are each independently 0, 1, 2, 3, 4, or 5; t¹, t² and t³ are each independently 0, 1, or 2; and wherein at least one of u¹, u², u³, t¹, t², and t³, is at least 1; and if present, R⁶ and R⁷ are each independently selected from the group consisting of hydrogen, —C₁₋₆alkyl, 3-10 membered carbocyclyl, and 3-10 membered heterocyclyl; and if present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, —C(O)C₁₋₆alkyl.

In some embodiments, the compound of Formula (I) is a dual inhibitor of a kinase enzyme and a bromodomain protein. In some embodiments, the compound of Formula (I) is a dual inhibitor of PI3K and BRD4.

In a further aspect, there is provided a pharmaceutical composition comprising a compound of Formula (I), salt, solvate or stereoisomer, and a pharmaceutically acceptable excipient.

In a further aspect, there is provided a method of inhibiting a kinase enzyme and a bromodomain protein, comprising contacting a compound of Formula (I), salt, solvate or stereoisomer, or the pharmaceutical composition, with a kinase enzyme and a bromodomain protein.

In a further aspect, there is provided the compound of Formula (I), salt, solvate or stereoisomer, or the pharmaceutical composition, for use in the prevention or treatment of a cancer.

In a further aspect, there is provided a method of preventing or treating cancer in a subject, comprising administering an effective amount of the compound of Formula (I), salt, solvate or stereoisomer, or the pharmaceutical composition, to the subject.

In a further aspect, there is provided the use of a compound of Formula (I), salt, solvate or stereoisomer, or of the pharmaceutical composition, in the manufacture of a medicament for use in preventing or treating a cancer in a subject.

### BRIEF DESCRIPTION OF THE DRAWINGS

FIG. 1 shows the results of the activity of Cpd 010 in an apoptosis assay against a panel of human multiple myeloma cell lines.

FIG. 2 shows Western Blot analysis of control (DMSO) and Cpd 010.

FIG. 3 shows the results of the cellular thermal shift assay (CETSA) of control (DMSO) and Cpd 010.

FIG. 4 shows the transciptional downregulation of MYC gene by Cpd 010.

FIG. 5 shows the results of the RNA-Seq and Gene Set Enrichment Analysis (GSEA) against MYC target genes by Cpd 010.

FIG. 6 shows the results of the chromatin immunoprecipitation and sequencing analysis of Cpd 010.

FIG. 7 shows the results of the apoptosis assay in BET-inhibitor-resistant cells.

FIG. 8 shows the synergy between PI3K and BET inhibitors and the energy score calculation.

FIG. 9 shows the results of the in vivo assessment of the maximum tolerated dose (MTD) of Cpd 010.

FIG. 10 shows the results of the in vivo therapeutic assessment of Cpd 010.

FIG. 11 shows the results of the in vivo assessment of PD-L1, PI3K activity, and tumour burden from a single dose of Cpd 010.

### DESCRIPTION

### General Definitions

Unless specifically defined otherwise, all technical and scientific terms used herein shall be taken to have the same meaning as commonly understood by one of ordinary skill in the art (e.g., chemistry, medicinal chemistry and the like).

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

As used herein, singular forms “a”, “an” and “the” include plural aspects, unless the context clearly indicates otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” refers to any organism susceptible to a disease or condition. For example, the subject can be a mammal, primate, livestock (e.g., sheep, cow, horse, pig), companion animal (e.g., dog, cat), or laboratory animal (e.g., mouse, rabbit, rat, guinea pig, hamster). In one example, the subject is a mammal. In one embodiment, the subject is human. In one embodiment, the disease or condition is cancer.

As used herein, the term “treating” includes alleviation of the symptoms associated with a specific disorder or condition and eliminating said symptoms. For example, as used herein, the term “treating cancer” refers to alleviating the symptoms associated with cancer and eliminating said symptoms. In one embodiment, the term “treating cancer” refers to a reduction in cancerous tumour size. In one embodiment, the term “treating cancer” refers to an increase in progression-free survival.

As used herein, the term “prevention” includes prophylaxis of the specific disorder or condition. For example, as used herein, the term “preventing cancer” refers to preventing the onset or duration of the symptoms associated with cancer. In one example, the term “preventing cancer” refers to slowing or halting the progression of the cancer. In one example, the term “preventing cancer” refers to slowing or preventing metastasis.

As would be understood by the person skilled in the art, a compound of Formula (I) or salt thereof would be administered in a therapeutically effective amount. The term “therapeutically effective amount”, as used herein, refers to a compound of Formula (I) or salt thereof being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a compound of Formula (I) or salt thereof being administered in an amount sufficient to result in a reduction of symptoms associated with cancer. In one embodiment, the term “therapeutically effective amount” refers to a compound of Formula (I) or salt thereof being administered in an amount sufficient to result in a reduction in cancerous tumour size.

The term, an “effective amount”, as used herein, refers to an amount of a compound of Formula (I) or salt thereof effective to achieve a desired pharmacologic effect or therapeutic improvement without undue adverse side effects or to achieve a desired pharmacologic effect or therapeutic improvement with a reduced side effect profile. By way of example only, therapeutically effective amounts may be determined by routine experimentation, including but not limited to a dose escalation clinical trial. The term “therapeutically effective amount” includes, for example, a prophylactically effective amount. In one embodiment, a prophylactically effective amount is an amount sufficient to prevent cancer. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. Thus, it is not always possible to specify an exact “effective amount”. However, an appropriate “effective amount” in any individual case may be determined by one of ordinary skill in the art using routine experimentation. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

The compounds of the present disclosure may contain chiral (asymmetric) centers or the molecule as a whole may be chiral. The individual stereoisomers (enantiomers and diastereoi-somers) and mixtures of these are within the scope of the present disclosure.

The following definitions apply to the terms as used throughout this specification, unless otherwise limited in specific instances.

As used herein, the term “halogen” means fluorine, chorine, bromine, or iodine.

As used herein, the term “alkyl” encompasses both straight chain (i.e., linear) and branched chain hydrocarbon groups. Examples of alkyl groups include methyl, ethyl, n-propyl, iso-propyl, n-butyl, t-butyl, i-butyl, sec-butyl, pentyl, and hexyl groups. In one example, the alkyl group is of one to six carbon atoms (i.e. C₁₋₆alkyl).

“Alkylene” or “alkylene chain” refers to a straight or branched divalent hydrocarbon chain linking the rest of the molecule to a radical group, consisting solely of carbon and hydro gen, containing no unsaturation and having from one to twelve carbon atoms, for example, methylene, ethylene, propylene, n butylene, and the like. The alkylene chain is attached to the rest of the molecule through a single bond and to the radical group through a single bond. The point of attachment of the alkylene chain to the rest of the molecule and to the radical group is through one carbon in the alkylene chain or through any two carbons within the chain. In some embodiments, an alkylene comprises one to eight carbon atoms (e.g., C₁₋₈alkylene). In some embodiments, an alkylene comprises one to five carbon atoms (e.g., C₁₋₅alkylene). In some embodiments, an alkylene comprises one to four carbon atoms (e.g., C₁₋₄alkylene). In some embodiments, an alkylene comprises one to three carbon atoms (e.g., C₁₋₃alkylene). In some embodiments, an alkylene comprises one to two carbon atoms (e.g., C₁₋₂alkylene). In some embodiments, an alkylene comprises one carbon atom (e.g., C₁alkylene).

As used herein, the term “alkoxy” refers to the group —O-alkyl, where “alkyl” is as described above. Examples of alkoxy groups include methoxy, ethoxy, propoxy, and butoxy groups. In one example, the alkoxy group is of one to six carbon atoms (i.e. —O—C₁₋₆alkyl).

As used herein, the term “alkenyl” refers to both straight and branched chain unsaturated hydrocarbon groups with at least one carbon-carbon double bond. Examples of alkenyl groups include ethenyl, propenyl, butenyl, pentenyl, and hexenyl groups. In one example, the alkenyl group is of two to six carbon atoms (i.e. C₂₋₆alkenyl).

As used herein, the term “alkynyl” refers to both straight and branched chain unsaturated hydrocarbon groups with at least one carbon-carbon triple bond. Examples of alkynyl groups include ethynyl, propynyl, butynyl, pentynyl, and hexynyl groups. In one example, the alkynyl group is of two to six carbon atoms (i.e. C₂₋₆alkynyl).

As used herein, the term “haloalkyl” refers to an alkyl group having at least one halogen substituent, where “alkyl” and “halogen” are as described above. Similarly, the term “dihaloalkyl” means an alkyl group having two halogen substituents, and the term “trihaloalkyl” means an alkyl group having three halogen substituents. Examples of haloalkyl groups include fluoromethyl, chloromethyl, bromomethyl, iodomethyl, fluoropropyl, and fluorobutyl groups. Examples of dihaloalkyl groups include difluoromethyl and difluoroethyl groups. Examples of trihaloalkyl groups include trifluoromethyl and trifluoroethyl groups. In one example, the haloalkyl group is of one to six carbon atoms (i.e. C₁₋₆haloalkyl).

As used herein, the terms “carbocyclyl” and “carbocycle” refer to a monovalent non-aromatic, saturated, or partially unsaturated, or aromatic ring having 3 to 12 carbon atoms (i.e., 3-12 membered carbocylyl) as a monocyclic ring. In one example, the carbocyclyl is a 3-10 membered carbocyclyl. A carbocyclyl group may, for example, be monocyclic or polycyclic (i.e. bicyclic, tricyclic). A polycyclic carbocyclyl group may contain fused rings. Examples of monocyclic carbocyclyl groups include, but are not limited to, cyclopropyl, cyclobutyl, cyclo-pentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, and the like. Examples of monocyclic, aromatic carbocyclyl group include, but are not limited to, phenyl and naphthalenyl.

As used herein, the term “heterocyclyl” refers to an aromatic or non-aromatic cyclic group which is analogous to a carbocyclyl group, but in which from one or more of the carbon atoms is/are replaced by one or more heteroatoms independently selected from nitrogen, oxy-gen, or sulfur. A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydro-quinolyl, and benzoazepanyl. Examples of monocyclic aromatic heterocyclyl groups (also referred to as monocyclic heteroaryl groups) include furanyl, thienyl, pyrrolyl, oxazolyl, thia-zolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinul, pyr-idopyrazinyl, benzoxazolyl, benzothiophenyl, benzimidazolyl, naphthyridinyl, quinolinyl, ben-zofuranyl, indolyl, indazolyl, benzothiazolyl, oxazolyl[4,5-b]pyridyl, pyridopyrimidinyl, iso-quinolinyl, and benzohydroxazole.

As used herein, the term “saturated” refers to a group where all available valence bonds of the backbone atoms are attached to other atoms Representative examples of saturated groups include, but are not limited to, butyl, cyclohexyl, piperidine, and the like.

As used herein, the term “unsaturated” refers to a group where at least one valence bond of two adjacent backbone atoms is not attached to other atoms. Representative examples include, but are not limited to, alkenes (e.g., —CH₂—CH₂CH═CH), phenyl, pyrrole, and the like.

As used herein, the term “substituted” refers to a group having one or more hydrogens or other atoms removed from a carbon or suitable heteroatom and replaced with a further group (i.e., substituent).

As used herein, the term “unsubstituted” refers to a group that does not have any further groups attached thereto or substituted therefore.

The present disclosure relates to compounds of Formula (I) and salts thereof. Salts may be formed in the case of embodiments of the compound of Formula (I) which contain a suitable acidic or basic group. Suitable salts of the compound of Formula (I) include those formed with organic or inorganic acids or bases.

As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccha-rate, formate, benzoate, glutamate, methanesulfonate, ethanesulfonate, benzenesulfonate, p-tol-uenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or tri-ethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport. In one example, the compound of Formula (I) is a dihydrochloride salt. In one example, the compound of Formula (I) is a hydrochloride salt.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of formula (I) and salts thereof.

As used herein, the term “stereoisomer” refers to compounds having the same molecular formula and sequence of bonded atoms (i.e., atom connectivity), though differ in the three-dimensional orientations of their atoms in space. As used herein, the term “enantiomers” refers to two compounds that are stereoisomers in that they are non-superimposable mirror images of one another. Relevant stereocenters may be donated with (R)- or (S)-configuration.

Those skilled in the art of organic and/or medicinal chemistry will appreciate that the compounds of Formula (I) and salts thereof may be present in amorphous form, or in a crystal line form. It will be understood that the present disclosure encompasses all forms and poly-morphs of the compounds of Formula (I) and salts thereof.

### Dual Inhibitors

The present disclosure provides compounds of Formula (I), or a pharmaceutically acceptable salt, solvate, or stereoisomer thereof:

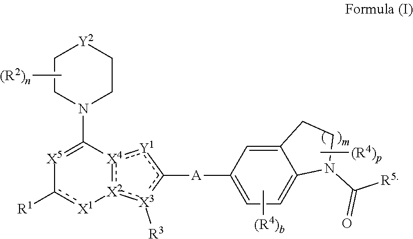

In the above Formula (I), X¹ can be C(R) or N; X² can be CH, C, or N; X³ can be C or N; X⁴ can be C or N; X⁵ can be C or N; Y¹ can be CH, N, or S; and Y² can be O, CH₂, or N(R).

In the above Formula (I), R is selected from the group consisting of hydrogen, halogen, and C₁₋₆alkyl.

In the above Formula (I), R¹, R², R³, and R⁴ can each be independently selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷). The alkyl, alkenyl, alkynyl, carbocyclyl or heterocyclyl groups can each be unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. Each —C₁₋₆alkyl can be unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶. In the above Formula (I), R⁵ can be selected from the group consisting of —C₁₋₆alkyl, —O(R⁶), 3-10 membered carbocyclyl, and 3-10 membered heterocyclyl.

In the above Formula (I), m can be 1, 2, or 3; n can be 0, 1, 2, 3, 4, 5, 6, 7, or 8; p can be 0, 1, 2, 3, 4, 5, 6, 7, or 8; and b can be 0, 1, 2, or 3.

In the above Formula (I), A can be a linker of Formula (II):

-[A¹]ᵤ₁-[Z¹]ₜ₁-[A²]ᵤ₂-[Z²]ₜ₂-[A³]ᵤ₃-[Z³]ₜ₃— Formula (II).

In the above Formula (II), A¹, A² and A³ can each be independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶.

In the above Formula (II), Z¹, Z² and Z³ can each be independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), and —C(O)OR⁶ or —C(O)R⁶.

In the above Formula (II), u¹, u² and u³ can each be independently 0, 1, 2, 3, 4, or 5; and t¹, t² and t³ can each be independently 0, 1, or 2. It will be appreciated that at least one of u¹, u², u³, t¹, t², and t³, is at least 1. For example, any one or more of u¹, u², u³, t¹, t², and t³, can be 1 or more. If present, R⁶ and R⁷ can each be independently selected from the group consisting of hydrogen, —C₁₋₆alkyl, 3-10 membered carbocyclyl, and 3-10 membered heterocyclyl. If present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

The present disclosure further provides compounds of Formula (I), or a pharmaceutically acceptable salt, solvate or stereoisomer thereof:

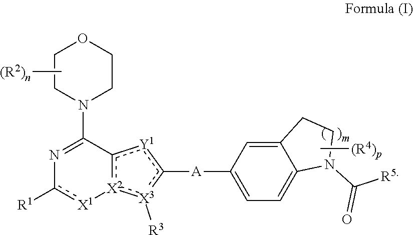

In the above Formula (I), X¹ can be C(R), CH, or N; X² can be CH, C or N; X³ can be C or N; and Y¹ can be CH, N or S.

In the above Formula (I), R is selected from the group consisting of hydrogen, halogen, and C₁₋₆alkyl, as described herein.

In the above Formula (I), R¹, R², R³, and R⁴ can each be independently selected from the group consisting of hydrogen, halogen, —C₁₋₆alkyl, —O—C₁₋₆alkyl, —C₁₋₆haloalkyl, —O—C₁-6haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷). The alkyl, alkenyl, alkynyl, carbocyclyl or heterocyclyl groups can each be unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. Each —C₁₋₆alkyl can be unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶. In the above Formula (I), R⁵ can be selected from the group consisting of —C₁₋₆alkyl, —O(R⁶), 3-10 membered carbocyclyl, and 3-10 membered heterocyclyl.

In the above Formula (I), m can be 1, 2, or 3; n can be 0, 1, 2, 3, 4, 5, 6, 7, or 8; and p can be 0, 1, 2, 3, 4, 5, 6, 7, or 8.

In the above Formula (I), A can be a linker of Formula (II):

[A¹]ᵤ₁-[Z¹]ₜ₁-[A²]ᵤ₂-[Z²]ₜ₂-[A³]ᵤ₃-[Z³]ₜ₃— Formula (II).

In the above Formula (II), A¹, A², and A³ can each be independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶.

In the above Formula (II), Z¹, Z², and Z³ can each be independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶.

In the above Formula (II), u¹, u², and u³ can each be independently 0, 1, 2, 3, 4, or 5; and t¹, t² and t³ can each be independently 0, 1, or 2. It will be appreciated that at least one of u¹, u², u³, t¹, t², and t³, is at least 1. For example, any one or more of u¹, u², u³, t¹, t², and t³, can be 1 or more. If present, R⁶ and R⁷ can each be independently selected from the group consisting of hydrogen, —C₁₋₆alkyl, 3-10 membered carbocyclyl, and 3-10 membered heterocyclyl. If present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

As used herein, the term “dual inhibitor” refers to the capacity of a single molecule to interact with at least two different protein targets in vitro or in vivo, including in the capacity to inhibit the activity or normal function of said targets, e.g., to inhibit binding or enzymatic activity. In one example, the compounds of Formula (I) are effective or capable of providing dual inhibitors of both kinase proteins and bromodomain proteins. In one example, the compounds of Formula (I) are effective or capable of providing dual inhibitors of both PI3K proteins and bromodomain proteins. That is, the compounds of Formula (I) are effective or capable of providing both PI3K inhibitors and bromodomain inhibitors.

PI3K belongs to the family of lipid kinases, which act on lipids by phosphorylating them on their hydroxyl substituents. It is this phosphorylation that can modify the function of a lipid. The phosphorylation state of phosphatidylinositol plays a major role in cellular signaling, such as in the insulin signaling pathway, and also has roles in endocytosis, exocytosis and other cell trafficking events. The phosphatidylinositol kinases are responsible for the phosphorylation of phosphatidylinositol species. This sub-family includes phosphoinositide 3-kinase (PI3K). PI3Ks are a family of enzymes involved in cellular functions such as cell growth, proliferation, differentiation, motility, survival and intracellular trafficking, which in turn implicates the PI3Ks in cancer. The PI3Ks may be further divided into Class I, Class II, Class III, and Class IV, based on primary structure, regulation, and in vivo lipid substrate specificity. The Class I PI3Ks include PI3Kα, PI3Kβ, and PI3Kγ.

As used herein, the term “PI3K inhibitor” refers to a compound capable of interacting with the normal or wild-type function of PI3K, i.e., enzymatic activity, in vivo and in vitro (e.g., PI3Kα, PI3Kβ, PI3Kγ).

Bromodomains are a protein domain that recognizes acetylated lysine residues, and are responsible for transducing the signal carried by acetylated lysine residues and translating it into various normal or abnormal phenotypes. An example of the bromodomain family is the BET (bromodomain and extraterminal domain) family. Members of the BET family include BRD2, BRD3, BRD4 and BRDT.

As used herein, the term “bromodomain inhibitor” refers to a compound capable of interacting with the normal or wild-type function of a bromodomain protein, i.e., enzymatic activity, in vivo and in vitro (e.g., BRD4).

In one example, the dual inhibitor has the capacity to interact with at least one kinase protein and at least one bromodomain protein. In one example, the dual inhibitor has the capacity to interact with at least one PI3K protein and at least one bromodomain protein. In one example, the dual inhibitor has the capacity to interact with at least one PI3K protein and at least one BET protein. In one example, the dual inhibitor has the capacity to interact with at least one PI3K protein and at least one BRD4 protein. In one example, the dual inhibitor has the capacity to interact with at least one PI3Kα protein and at least one BRD4 protein. In one example, the dual inhibitor has the capacity to interact with at least one PI3Kβ protein and at least one BRD4 protein. In one example, the dual inhibitor has the capacity to interact with at least one PI3Kγ protein and at least one BRD4 protein. In one example, the dual inhibitor has the capacity to interact with at least one PI3Kδ protein and at least one BRD4 protein.

Compounds of Formula (I) will be described with reference to the left-hand side of the compound (i.e., the portion of the compound lying left of the -A- linker), the right-hand side of the compound (i.e., the portion of the compound lying right of the -A- linker), and the linker -A-.

### Left-Hand Side of Formula (I)

In one example, the compound of Formula (I) is:

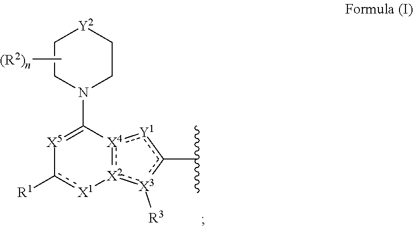

wherein
   X¹ is C(R) or N;
   X² is CH, C or N;
   X³ is C or N;
   X⁴ is C or N;
   X⁵ is C or N;
   Y¹ is CH, N or S;
   Y² is O, CH₂, or N(R);
   R is selected from the group consisting of hydrogen, halogen, and C₁₋₆alkyl;
   R¹, R², and R³ are each independently selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
      wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
         wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
   n is 0, 1, 2, 3, 4, 5, 6, 7, or 8;
   if present, R⁶ and R⁷ are each independently selected from the group consisting of hydrogen, —C₁₋₆alkyl, 3-10 membered carbocyclyl, and 3-10 membered heterocyclyl; and
   if present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

In the above Formula (I), R¹, R², R³, X², X³, and Y¹ are as described herein. Further, in the above Formula (I), in one example, X¹ is C(R). In one example, X¹ is N. In one example, X⁴ is C. In one example, X⁴ is N. In one example, X⁵ is C. In one example, X⁵ is N. In one example, Y² is 0. In one example, Y² is CH₂. In one example, Y² is N(R). In one example, R is hydrogen. In one example, R is halogen. Accordingly, in one example, R is chlorine. In one example, R is bromine. In one examine, R is iodine. In one example, R is a C₁₋₆alkyl group.

In one example, the compound of Formula (I) is:

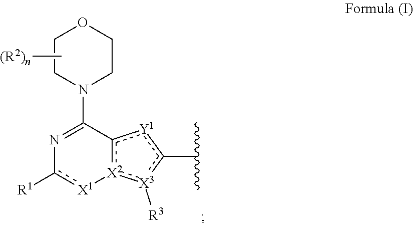

wherein
X¹ is C(R), CH or N;
X² is CH, C or N;
X³ is C or N;
Y¹ is CH, N or S;
   R is selected from the group consisting of hydrogen, halogen, and C₁₋₆alkyl;
R¹, R² and R³ are each independently selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
   wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
      wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
n is 0, 1, 2, 3, 4, 5, 6, 7, or 8; and
if present, R⁶ and R⁷ are each independently selected from the group consisting of hydrogen, —C₁₋₆alkyl, 3-10 membered carbocyclyl, and 3-10 membered heterocyclyl.

In one example, R¹ is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷); wherein said alkyl, alkenyl, alkynyl, carbocyclyl or heterocyclyl are each unsubstituted or substituted with one or more substituents each inde pendently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and wherein each —C₁₋₆alkyl is unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶; and if pre sent, R⁶ and R⁷ are each independently selected from the group consisting of hydrogen, —C₁₋₆alkyl, 3-10 membered carbocyclyl, and 3-10 membered heterocyclyl.

In one example, R¹ is hydrogen. In one example, R¹ is —C₁₋₆alkyl. In one example, R¹ is a 3-10 membered carbocyclyl. In one example, R¹ is a 3-10 membered heterocyclyl. In one example, R¹ is a 3-10 membered carbocyclyl, wherein said carbocyclyl is substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. In one example, R¹ is a 3-10 membered heterocyclyl, wherein said heterocyclyl is substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. In one example, R¹ is a 3-10 membered heterocyclyl, wherein said heterocyclyl is substituted with one or more substituents each independently selected from the group consisting of halogen, —C₁₋₆haloalkyl, and —N(R⁶)(R⁷). In one example, R¹ is a 3-10 membered heterocyclyl, wherein said heterocyclyl is substituted with one or more halogen substituents. In one example, R¹ is a 3-10 membered heterocyclyl, wherein said heterocyclyl is substituted with one or more —C₁₋₆haloalkyl substituents. In one example, R¹ is a 3-10 membered heterocyclyl, wherein said heterocyclyl is substituted with one or more —N(R⁶)(R⁷) substituents.

In one example, R¹ is a 3-10 membered heterocyclyl, wherein said heterocyclyl is substituted with —NH₂. In one example, R¹ is a 3-10 membered heterocyclyl, wherein said heterocyclyl is substituted with —CF₂. In one example, R¹ is a 3-10 membered heterocyclyl, wherein said heterocyclyl is substituted with —CF₃. In one example, R¹ is a 3-10 membered heterocyclyl, wherein said heterocyclyl is substituted with —CF₃ and —NH₂.

In one example, R¹ is selected from the group consisting of:

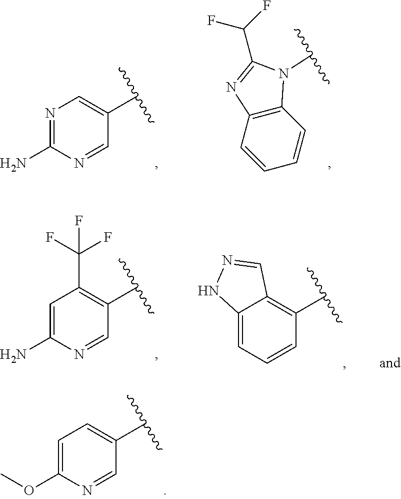

In one example, R¹ is selected from the group consisting of:

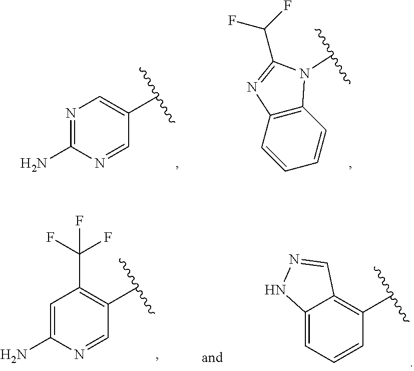 [SMILES: *c1cnc(N)nc1 ; *n1c(C(F)F)nc2ccccc21 ; *c1cnc(N)cc1C(F)(F)F ; *c1cccc2[nH]ncc12]

In one example, R² of Formula (I) is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷). The alkyl, alkenyl, alkynyl, carbocyclyl or heterocyclyl groups are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. Each —C₁₋₆alkyl is unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶. In one example, R² is hydrogen.

In one example, n of Formula (I) is 0, 1, 2, 3, 4, 5, 6, 7, or 8. In Formula (I), it will be appreciated that n designates the number of R² substituents. In one example, n of Formula (I) is 0. In the instance where n of Formula (I) is 0, it will be understood that the R² substituent is not present in Formula (I). In one example, n of Formula (I) is 1, and one R² substituent is present in Formula (I). In one example, n of Formula (I) is 2, and two R² substituents are present in Formula (I). In one example, n of Formula (I) is 3, and three R² substituents are present in Formula (I). In one example, n of Formula (I) is 4, and four R² substituents are present in Formula (I). In one example, n of Formula (I) is 5, and five R² substituents are present in Formula (I). In one example, n of Formula (I) is 6, and six R² substituents are present in Formula (I). In one example, n of Formula (I) is 7, and seven R² substituents are present in Formula (I). In one example, n of Formula (I) is 8, and eight R² substituents are present in Formula (I).

In one example, n is 1, 2, 3, 4, 5, 6, 7, or 8, and the R² substituent(s) is be attached to any carbon, and in any configuration, on the designated ring of Formula (I).

In one example, when n is 2, the two R² substituents present in Formula (I), may be attached to the same or different carbon atoms of the designated ring:

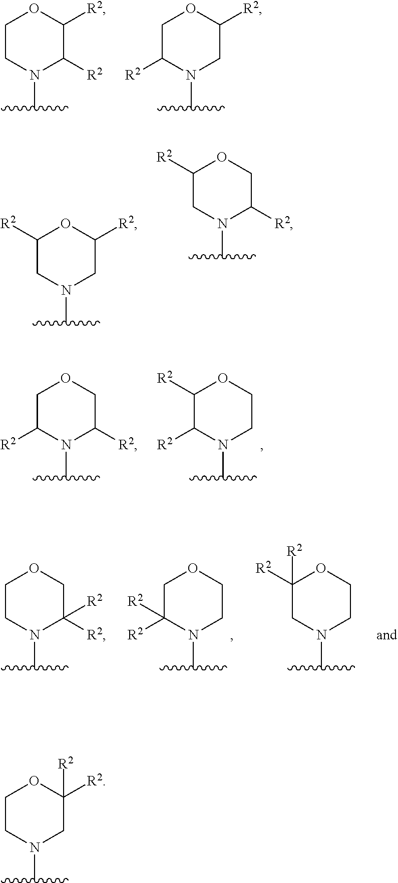

The same will be understood when n is 3, 4, 5, 6, 7, or 8.

In one example, R³ of Formula (I) is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷). The alkyl, alkenyl, alkynyl, carbocyclyl or heterocyclyl groups are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. Each —C₁₋₆alkyl is unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶.

In one example, R³ is hydrogen. In one example, R³ is —C₁₋₆alkyl. In one example, R³ is —CH₃.

In one example, X¹ is C(R), CH, or N. In one example, X¹ is C(R). In one example, X¹ is CH. In one example, X¹ is N.

In one example, X² is CH, C or N. In one example, X² is CH. In one example, X² is C.

In one example, X² is N.

In one example, X³ C or N. In one example, X³ is C. In one example, X³ is N.

In one example, Y¹ is CH, N or S. In one example, Y¹ is CH. In one example, Y¹ is N. In one example, Y¹ is S.

In one example, X¹ is C(R), X² is N, X³ is C, and Y¹ is N, wherein R is as described herein. In one example, X¹ is CH, X² is N, X³ is C, and Y¹ is N. In one example, X¹ is N, X² is C, X³ is C, and Y¹ is S. In one example, X¹ is CH, X² is C, X³ is N, and Y¹ is CH, and R³ is not present. In one example, X¹ is N, X² is N, X³ is C, and Y¹ is CH.

In one example, the compound of Formula (I) is:

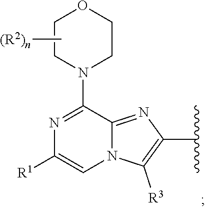

wherein R¹, R², and R³ are as described herein.

In one example, the compound of Formula (I) is:

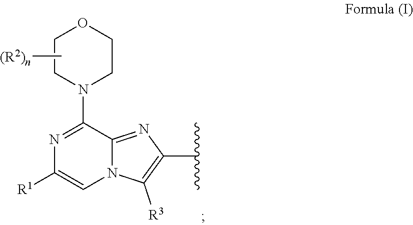

wherein R¹ is a 3-10 membered heterocyclyl, wherein said heterocyclyl is substituted with one or more substituents each independently selected from the group consisting of halogen, —C₁₋₆haloalkyl, and —N(R⁶)(R⁷); and R², R³, and n are as described herein.

In one example, the compound of Formula (I) is:

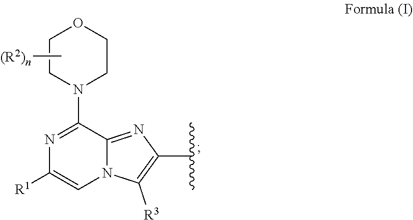

wherein R¹ is a 3-10 membered heterocyclyl, wherein said heterocyclyl is substituted with halogen; and R², R³, and n are as described herein.

In one example, the compound of Formula (I) is:

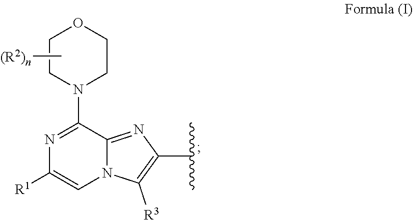

wherein R¹ is a 3-10 membered heterocyclyl, wherein said heterocyclyl is substituted with —C₁₋₆haloalkyl; and R², R³, and n are as described herein.

In one example, the compound of Formula (I) is:

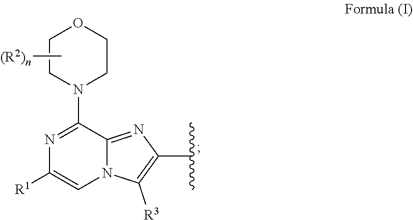

wherein R¹ is a 3-10 membered heterocyclyl, wherein said heterocyclyl is substituted with —NH₂; and R², R³, and n are as described herein.

In one example, the compound of Formula (I) is:

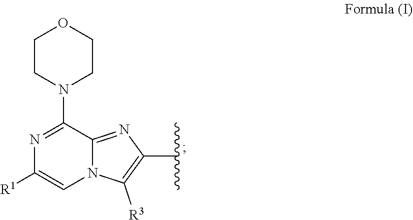

wherein R¹ is a 3-10 membered heterocyclyl, wherein said heterocyclyl is substituted with one or more substituents each independently selected from the group consisting of halogen, —C₁₋₆haloalkyl, and —N(R⁶)(R⁷); R³ is hydrogen or —CH₃; and if present, R⁶ and R⁷ are as described herein.

In one example, the compound of Formula (I) is:

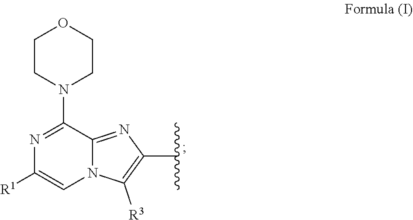

wherein R¹ is selected from the group consisting of:

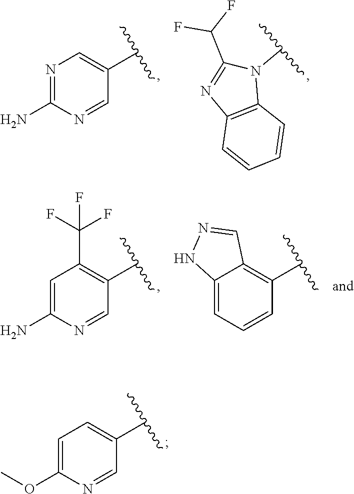

R³ is hydrogen or —CH₃; and if present, R⁶ and R⁷ are as described herein.

In one example, the compound of Formula (I) is:

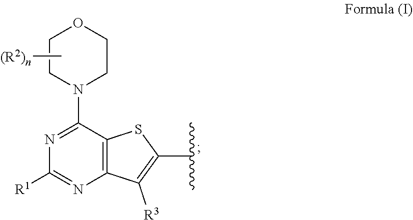

wherein R¹, R², R³, and n are as described herein.

In one example, the compound of Formula (I) is:

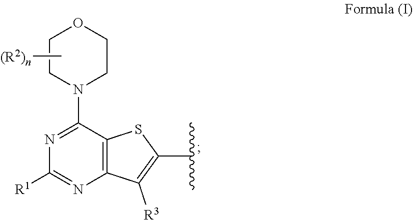

wherein R¹ is a 3-10 membered heterocyclyl, wherein said heterocyclyl is substituted with one or more substituents each independently selected from the group consisting of halogen, —C₁₋₆haloalkyl, and —N(R⁶)(R⁷); and R² is as described herein; R³ is —CH₃; and n is as described herein.

In one example, the compound of Formula (I) is:

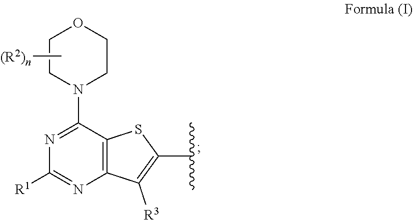

wherein R¹ is a 3-10 membered heterocyclyl, wherein said heterocyclyl is substituted with halogen; R² is as described herein; R³ is —CH₃; and n is as described herein.

In one example, the compound of Formula (I) is:

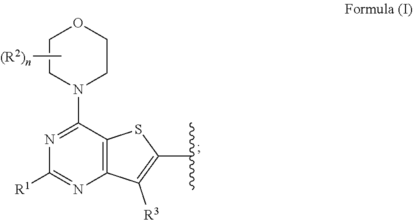

wherein R¹ is a 3-10 membered heterocyclyl, wherein said heterocyclyl is substituted with —C₁₋₆haloalkyl; R² is as described herein; R³ is hydrogen or —CH₃; and n is as described herein.

In one example, the compound of Formula (I) is:

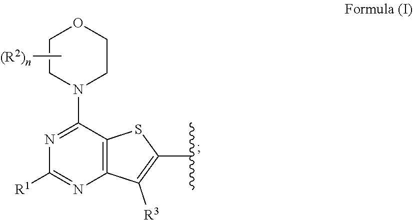

wherein R¹ is a 3-10 membered heterocyclyl, wherein said heterocyclyl is substituted with —NH₂; R² is as described herein; R³ is hydrogen or —CH₃; and n is as described herein.

In one example, the compound of Formula (I) is:

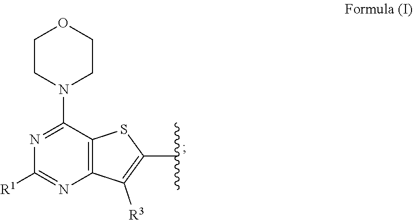

wherein R¹ is a 3-10 membered heterocyclyl, wherein said heterocyclyl is substituted with one or more substituents each independently selected from the group consisting of halogen, —C₁₋₆haloalkyl, and —N(R⁶)(R⁷); and R³ is hydrogen or —CH₃.

In one example, the compound of Formula (I) is:

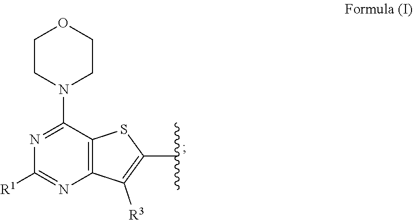

wherein R¹ is selected from the group consisting of:

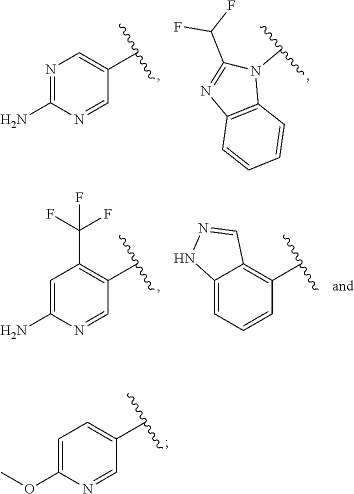

and R³ is hydrogen or —CH₃.

### Right-Hand Side of Formula (I)

In one example, the compound of Formula (I) is:

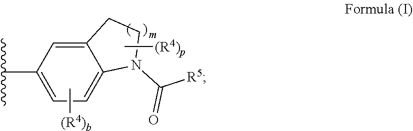

wherein R⁴ is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷); wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and wherein each —C₁₋₆alkyl is unsubstituted or sub stituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
m is 1, 2, or 3;
p is 0, 1, 2, 3, 4, 5, 6, 7, or 8;
b is 0, 1, 2, or 3; and if present, R⁶ and R⁷ are each independently selected from the group consisting of hydrogen, —C₁₋₆alkyl, 3-10 membered carbocyclyl, and 3-10 membered heterocyclyl.

In Formula (I), b can be 0, 1, 2, or 3. In Formula (I), it will be appreciated that b designates the number of R⁴ substituents present on the aryl ring. In one example, b of Formula (I) is 0. In the instance where p of Formula (I) is 0, it will be understood that the R⁴ substituent is not present in Formula (I). In one example, b is 1. In one example, b is 2. In one example, b is 3.

In one example, the compound of Formula (I) is:

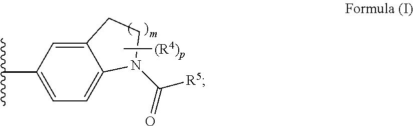

wherein R⁴ is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷); wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and wherein each —C₁₋₆alkyl is unsubstituted or sub stituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
m is 1, 2, or 3;
p is 0, 1, 2, 3, 4, 5, 6, 7, or 8; and
if present, R⁶ and R⁷ are each independently selected from the group consisting of hy drogen, —C₁₋₆alkyl, 3-10 membered carbocyclyl, and 3-10 membered heterocyclyl.

In one example, m of Formula (I) is 1, 2, or 3.

In one example, R⁴ of Formula (I) is selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷). The alkyl, alkenyl, alkynyl, carbocyclyl or heterocyclyl groups are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶. Each —C₁₋₆alkyl is unsubstituted or substituted with one or more substituents each independently selected from —OR⁶, —N(R⁶)(R⁷), —C(O)N(R⁶)(R⁷), —N(R⁶)C(O)(R⁷), —C(O)OR⁶, and —C(O)R⁶.
In one example, R⁴ is hydrogen. In one example, R⁴ is —C₁₋₆alkyl. In one example, R⁴ is —CH₃.

In one example, p of Formula (I) is 0, 1, 2, 3, 4, 5, 6, 7, or 8. In Formula (I), it will be appreciated that p designates the number of R⁴ substituents. In one example, p of Formula (I) is 0. In the instance where p of Formula (I) is 0, it will be understood that the R⁴ substituent is not present in Formula (I). In one example, p of Formula (I) is 1, and one R⁴ substituent is present in Formula (I). In one example, p of Formula (I) is 2, and two R⁴ substituents are present in Formula (I). In one example, p of Formula (I) is 3, and three R⁴ substituents are present in Formula (I). In one example, p of Formula (I) is 4, and four R⁴ substituents are present in Formula (I). In one example, p of Formula (I) is 5, and five R⁴ substituents are present in Formula (I). In one example, p of Formula (I) is 6, and six R⁴ substituents are present in Formula (I). In one example, p of Formula (I) is 7, and seven R⁴ substituents are present in Formula (I). In one example, p of Formula (I) is 8, and eight R⁴ substituents are present in Formula (I).

In one example, p is 1, 2, 3, 4, 5, 6, 7, or 8, and the R⁴ substituent(s) is attached to any carbon, and in any configuration, on the designated ring of Formula (I). It will be understood that for p to be 5 or 6, m must be at least 2. It will also be understood that for p to be 7 or 8, m must be 3.

In one example, R⁵ of Formula (I) is selected from the group consisting of —C₁₋₆alkyl, —O(R⁶), 3-10 membered carbocyclyl, and 3-10 membered heterocyclyl. In one example, R⁵ of Formula (I) is —C₁₋₆alkyl. In one example, R⁵ of Formula (I) is —O(R⁶). In one example, R⁵ of Formula (I) is 3-10 membered carbocyclyl. In one example, R⁵ of Formula (I) is 3-10 membered heterocyclyl.

In one example, R⁵ is hydrogen. In one example, R⁵ is —C₁₋₆alkyl. In one example, R⁵ is —CH₃. In one example, R⁵ is —OR⁶, wherein R⁶ is selected from the group consisting of hydrogen, —C₁₋₆alkyl, 3-10 membered carbocyclyl, and 3-10 membered heterocyclyl. In one example, R⁵ is —OR⁶, wherein R⁶ is hydrogen. In one example, R⁵ is —OR⁶, wherein R⁶ is —C₁₋₆alkyl. In one example, R⁵ is —OR⁶, wherein R⁶ is 3-10 membered carbocyclyl. In one example, R⁵ is —OR⁶, wherein R⁶ is 3-10 membered heterocyclyl. In one example, R⁵ is —OR⁶, wherein R⁶ is —CH₃. In one example, R⁵ is —OCH₃.

In one example, m is 1, and p is 0, 1, 2, 3, or 4. In one example, m is 2, and p is 0, 1, 2, 3, 4, 5 or 6. In one example, m is 3, and p is 0, 1, 2, 3, 4, 5, 6, 7, or 8.

In one example, m is 1:

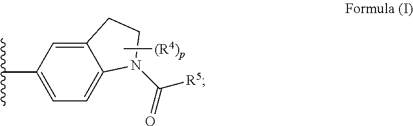

wherein R⁴, R⁵, and p are as described herein.

In one example, m is 2:

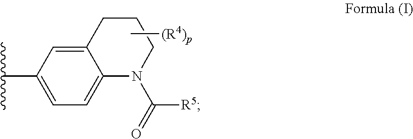

wherein R⁴, R⁵, and p are as described herein.
   In one example, m is 3:

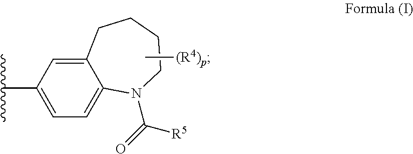

wherein R⁴, R⁵, and p are as described herein.

In one example, the compound of Formula (I) is:

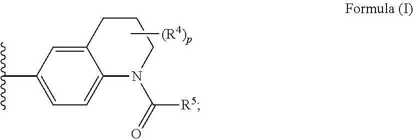

wherein R⁴, R⁵, and p are as described herein.

In one example, the compound of Formula (I) is:

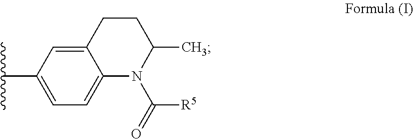

and R⁵ is selected from the group consisting of —C₁₋₆alkyl, —O(R⁶), 3-10 membered carbocyclyl, and 3-10 membered heterocyclyl; and R⁶ is as described herein.

In one example, the compound of Formula (I) is selected from the group consisting of:

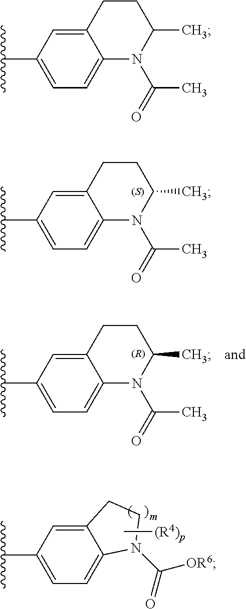

wherein R⁴, m, R⁶, and p are as described herein.

In one example, the compound of Formula (I) is:

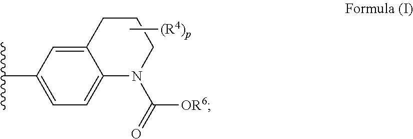

wherein R⁴, R⁶, and p are as described herein.

In one example, the compound of Formula (I) is:

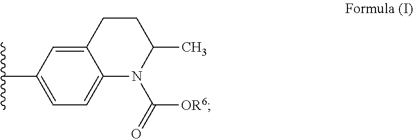

wherein R⁶ is selected from the group consisting of hydrogen, —C₁₋₆alkyl, 3-10 membered carbocyclyl, and 3-10 membered heterocyclyl.

In one example, the compound of Formula (I) is:

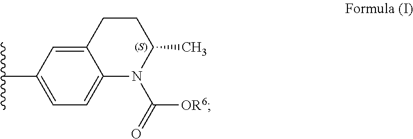

wherein R⁶ is as described herein.

In one example, the compound of Formula (I) is:

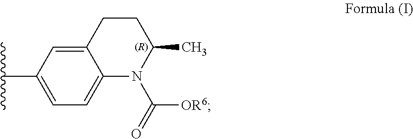

wherein R⁶ is as described herein.

In one example, the compound of Formula (I) is:

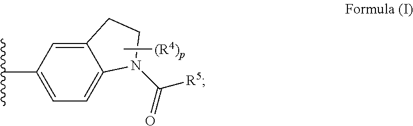

wherein R⁴, R⁵, and p are as described herein.

In one example, the compound of Formula (I) is:

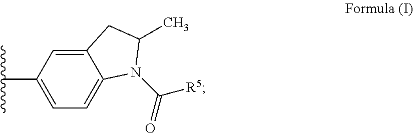

R⁵ is selected from the group consisting of —C₁₋₆alkyl, —O(R⁶), 3-10 membered carbocyclyl, and 3-10 membered heterocyclyl; and R⁶ is as described herein.

In one example, the compound of Formula (I) is selected from the group consisting of:

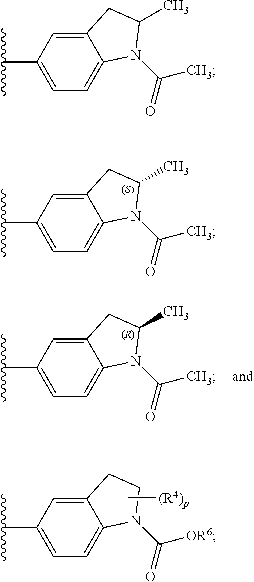

wherein R⁴ and p are as described herein.

In one example, the compound of Formula (I) is selected from the group consisting of:

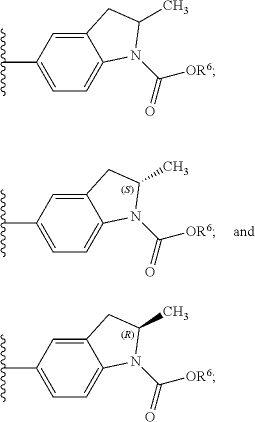

wherein R⁶ is as described herein.

### Linker A

In one example, -A- is a linker of Formula (II):

-[A¹]ᵤ₁-[Z¹]ₜ₁-[A²]ᵤ₂-[Z²]ₜ₂-[A³]ᵤ₃-[Z³]ₜ₃— Formula (II);
wherein A¹, A², and A³ are each independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶;
u¹, u², and u³ are each independently 0, 1, 2, 3, 4, or 5;
t¹, t², and t³ are each independently 0, 1, or 2; and
wherein at least one of u¹, u², u³, t¹, t², and t³, is at least 1; and
if present, R⁶ and R⁷ are each independently selected from the group consisting of hydrogen, —C₁₋₆alkyl, 3-10 membered carbocyclyl, and 3-10 membered heterocyclyl; and if present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

In one example, u¹ is 0, 1, 2, 3, 4, or 5. In one example, u¹ is 0, and it will be understood that no A¹ is present in the linker of Formula (II). Similarly, in one example, u¹ is 1, and -A¹- is present in the linker. Similarly, in one example, u¹ is 2, and -A¹-A¹- is present in the linker. Similarly, in one example, u¹ is 3, and -A¹-A¹-A¹- is present in the linker. Similarly, in one example, u¹ is 4, and A¹-A¹-A¹-A¹- is present in the linker. Similarly, in one example, u¹ is 5, and -A¹-A¹-A¹-A¹-A¹- is present in the linker.

In one example, u² is 0, 1, 2, 3, 4, or 5. In one example, u² is 0, and it will be understood that no A² is present in the linker of Formula (II). Similarly, in one example, u² is 1, and -A²- is present in the linker. Similarly, in one example, u² is 2, and -A²-A²- is present in the linker. Similarly, in one example, u² is 3, and -A²-A²-A²- is present in the linker. Similarly, in one example, u² is 4, and -A²-A²-A²-A² is present in the linker. Similarly, in one example, u² is 5, and -A²-A²-A²-A²-A²- is present in the linker.

In one example, u³ is 0, 1, 2, 3, 4, or 5. In one example, u³ is 0, and it will be understood that no A³ is present in the linker of Formula (II). Similarly, in one example, u³ is 1, and -A³- is present in the linker. Similarly, in one example, u³ is 2, and -A³-A³- is present in the linker. Similarly, in one example, u³ is 3, and -A³-A³-A³- is present in the linker. Similarly, in one example, u³ is 4, and -A³-A³-A³-A³- is present in the linker. Similarly, in one example, u³ is 5, and A³-A³-A³-A³-A³- is present in the linker.

It will be appreciated that when, for example, u¹ is 2 and -A¹-A¹- is present in the linker, each A¹ is independently selected from the group consisting of —C₁₋₆alkylene-, —C(O)—, —C(O)N(R⁶)—, —N(R⁶)C(O)—, —N(R⁶)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶. In one example, u¹ is 2 and -A¹-A¹- is present in the linker as —C(O)—C₁₋₆alkylene-. That is, the one or more A¹ present in the linker are independent of one another.

In one example, t¹ is 0, 1, or 2. In one example, t¹ is 0, and it will be understood that no Z¹ is present in the linker of Formula (II). Similarly, in one example, t¹ is 1, and —Z¹— is present in the linker. Similarly, in one example, t¹ is 2, and —Z¹—Z¹— is present in the linker.

In one example, t² is 0, 1, or 2. In one example, t² is 0, and it will be understood that no Z² is present in the linker of Formula (II). Similarly, in one example, t² is 1, and —Z²— is present in the linker. Similarly, in one example, t² is 2, and —Z²—Z²— is present in the linker.

In one example, t³ is 0, 1, or 2. In one example, t³ is 0, and it will be understood that no Z³ is present in the linker of Formula (II). Similarly, in one example, t³ is 1, and —Z³— is present in the linker. Similarly, in one example, t³ is 2, and —Z³—Z³— is present in the linker.

It will be appreciated that when, for example, t¹ is 2 and —Z¹—Z¹— is present in the linker, each Z¹ is independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶. In one example, t¹ is 2 and —Z¹—Z¹— is present in the linker as -3-10 membered carbocyclyl-3-10 membered heterocyclyl-. That is, the one or more Z¹ present in the linker are independent of one another.

In one example, at least one of u¹, u², u³, t¹, t², and t³ is at least 1. That is, the linker of Formula (II) is present in Formula (I) (i.e., there is not direct attachment of the left-hand side to the right-hand side). That is, in one example, A is present in Formula (I).

In one example, Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents inde pendently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z¹ is selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z² is selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z³ is selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z¹ is 3-10 membered carbocyclyl. In one example, Z¹ is 3-10 membered heterocyclyl. In one example, Z² is 3-10 membered carbocyclyl. In one example, Z² is 3-10 membered heterocyclyl. In one example, Z³ is 3-10 membered carbocyclyl. In one example, Z³ is 3-10 membered heterocyclyl.

In one example, Z¹ is a 3-10 membered unsaturated heterocyclyl. In one example, Z¹ is a 3-10 membered unsaturated carbocyclyl. In one example, Z¹ is a 5-6 membered unsaturated heterocyclyl. In one example, Z¹ is a 5-6 membered unsaturated carbocyclyl. In one example, Z¹ is a 5 membered unsaturated heterocyclyl. In one example, Z¹ is a 6 membered unsaturated heterocyclyl. In one example, Z¹ is a 3-10 membered partially or fully saturated heterocyclyl. In one example, Z¹ is a 3-10 membered partially or fully saturated carbocyclyl. In one example, Z¹ is a 5-6 membered partially or fully saturated heterocyclyl. In one example, Z¹ is a 5-6 membered partially or fully saturated carbocyclyl. In one example, Z¹ is a 5 membered partially or fully saturated heterocyclyl. In one example, Z¹ is a 6 membered partially or fully saturated heterocyclyl.

In one example, Z² is a 3-10 membered unsaturated heterocyclyl. In one example, Z² is a 3-10 membered unsaturated carbocyclyl. In one example, Z² is a 5-6 membered unsaturated heterocyclyl. In one example, Z² is a 5-6 membered unsaturated carbocyclyl. In one example, Z² is a 5 membered unsaturated heterocyclyl. In one example, Z² is a 6 membered unsaturated heterocyclyl. In one example, Z² is a 3-10 membered partially or fully saturated heterocyclyl. In one example, Z² is a 3-10 membered partially or fully saturated carbocyclyl. In one example, Z² is a 5-6 membered partially or fully saturated heterocyclyl. In one example, Z² is a 5-6 membered partially or fully saturated carbocyclyl. In one example, Z² is a 5 membered partially or fully saturated heterocyclyl. In one example, Z² is a 6 membered partially or fully saturated heterocyclyl.

In one example, Z³ is a 3-10 membered unsaturated heterocyclyl. In one example, Z³ is a 3-10 membered unsaturated carbocyclyl. In one example, Z³ is a 5-6 membered unsaturated heterocyclyl. In one example, Z³ is a 5-6 membered unsaturated carbocyclyl. In one example, Z³ is a 5 membered unsaturated heterocyclyl. In one example, Z³ is a 6 membered unsaturated heterocyclyl. In one example, Z³ is a 3-10 membered partially or fully saturated heterocyclyl. In one example, Z³ is a 3-10 membered partially or fully saturated carbocyclyl. In one example, Z³ is a 5-6 membered partially or fully saturated heterocyclyl. In one example, Z³ is a 5-6 membered partially or fully saturated carbocyclyl. In one example, Z³ is a 5 membered partially or fully saturated heterocyclyl. In one example, Z³ is a 6 membered partially or fully saturated heterocyclyl.

In one example, Z¹ is 3-10 membered carbocyclyl substituted with one or more halogen. In one example, Z¹ is 3-10 membered heterocyclyl substituted with one or more halogen. In one example, Z² is 3-10 membered carbocyclyl substituted with one or more halogen. In one example, Z² is 3-10 membered heterocyclyl substituted with one or more halogen. In one ex ample, Z³ is 3-10 membered carbocyclyl substituted with one or more halogen. In one example, Z³ is 3-10 membered heterocyclyl substituted with one or more halogen.

In one example, Z¹ is 3-10 membered carbocyclyl substituted with one or more —OH. In one example, Z¹ is 3-10 membered heterocyclyl substituted with one or more —OH. In one ex ample, Z² is 3-10 membered carbocyclyl substituted with one or more —OH. In one example, Z² is 3-10 membered heterocyclyl substituted with one or more —OH. In one example, Z³ is 3-10 membered carbocyclyl substituted with one or more —OH. In one example, Z³ is 3-10 membered heterocyclyl substituted with one or more —OH.

In one example, Z¹ is 3-10 membered carbocyclyl substituted with one or more ═O. In one example, Z¹ is 3-10 membered heterocyclyl substituted with one or more ═O. In one exam ple, Z² is 3-10 membered carbocyclyl substituted with one or more ═O. In one example, Z² is 3-10 membered heterocyclyl substituted with one or more ═O. In one example, Z³ is 3-10 membered carbocyclyl substituted with one or more ═O. In one example, Z³ is 3-10 membered heterocyclyl substituted with one or more ═O.

In one example, Z¹ is 3-10 membered carbocyclyl substituted with one or more —C₁₋₆alkyl. In one example, Z¹ is 3-10 membered heterocyclyl substituted with one or more —C₁₋₆alkyl. In one example, Z² is 3-10 membered carbocyclyl substituted with one or more —C₁₋₆alkyl. In one example, Z² is 3-10 membered heterocyclyl substituted with one or more —C₁₋₆alkyl. In one example, Z³ is 3-10 membered carbocyclyl substituted with one or more —C₁₋₆alkyl. In one example, Z³ is 3-10 membered heterocyclyl substituted with one or more —C₁₋₆alkyl.

In one example, Z¹ is 3-10 membered carbocyclyl substituted with one or more —O—C₁₋₆alkyl. In one example, Z¹ is 3-10 membered heterocyclyl substituted with one or more —O—C₁₋₆alkyl. In one example, Z² is 3-10 membered carbocyclyl substituted with one or more —O—C₁₋₆alkyl. In one example, Z² is 3-10 membered heterocyclyl substituted with one or more —O—C₁₋₆alkyl. In one example, Z³ is 3-10 membered carbocyclyl substituted with one or more —O—C₁₋₆alkyl. In one example, Z³ is 3-10 membered heterocyclyl substituted with one or more —O—C₁₋₆alkyl.

In one example, Z¹ is 3-10 membered carbocyclyl substituted with one or more —N(R⁶)(R⁷). In one example, Z¹ is 3-10 membered heterocyclyl substituted with one or more N(R⁶)(R⁷). In one example, Z² is 3-10 membered carbocyclyl substituted with one or more N(R⁶)(R⁷). In one example, Z² is 3-10 membered heterocyclyl substituted with one or more N(R⁶)(R⁷). In one example, Z³ is 3-10 membered carbocyclyl substituted with one or more N(R⁶)(R⁷). In one example, Z³ is 3-10 membered heterocyclyl substituted with one or more N(R⁶)(R⁷).

In one example, Z¹ is 3-10 membered carbocyclyl substituted with one or more —C(O)N(R⁶)(R⁷). In one example, Z¹ is 3-10 membered heterocyclyl substituted with one or more —C(O)N(R⁶)(R⁷). In one example, Z² is 3-10 membered carbocyclyl substituted with one or more —C(O)N(R⁶)(R⁷). In one example, Z² is 3-10 membered heterocyclyl substituted with one or more —C(O)N(R⁶)(R⁷). In one example, Z³ is 3-10 membered carbocyclyl substituted with one or more —C(O)N(R⁶)(R⁷). In one example, Z³ is 3-10 membered heterocyclyl substituted with one or more —C(O)N(R⁶)(R⁷).

In one example, Z¹ is 3-10 membered carbocyclyl substituted with one or more —N(R⁶)C(O)(R⁷). In one example, Z¹ is 3-10 membered heterocyclyl substituted with one or more —N(R⁶)C(O)(R⁷). In one example, Z² is 3-10 membered carbocyclyl substituted with one or more —N(R⁶)C(O)(R⁷). In one example, Z² is 3-10 membered heterocyclyl substituted with one or more —N(R⁶)C(O)(R⁷). In one example, Z³ is 3-10 membered carbocyclyl substituted with one or more —N(R⁶)C(O)(R⁷). In one example, Z³ is 3-10 membered heterocyclyl substituted with one or more —N(R⁶)C(O)(R⁷).

In one example, Z¹ is 3-10 membered carbocyclyl substituted with one or more —C(O)OR⁶. In one example, Z¹ is 3-10 membered heterocyclyl substituted with one or more —C(O)OR⁶. In one example, Z² is 3-10 membered carbocyclyl substituted with one or more —C(O)OR⁶. In one example, Z² is 3-10 membered heterocyclyl substituted with one or more —C(O)OR⁶. In one example, Z³ is 3-10 membered carbocyclyl substituted with one or more —C(O)OR⁶. In one example, Z³ is 3-10 membered heterocyclyl substituted with one or more —C(O)OR⁶.

In one example, Z¹ is 3-10 membered carbocyclyl substituted with one or more —C(O)R⁶. In one example, Z¹ is 3-10 membered heterocyclyl substituted with one or more —C(O)R⁶. In one example, Z² is 3-10 membered carbocyclyl substituted with one or more —C(O)R⁶. In one example, Z² is 3-10 membered heterocyclyl substituted with one or more —C(O)R⁶. In one ex ample, Z³ is 3-10 membered carbocyclyl substituted with one or more —C(O)R⁶. In one example, Z³ is 3-10 membered heterocyclyl substituted with one or more —C(O)R⁶.

It will be understood that each Z group, for example one Z¹ group, may be substituted with one or more different substituents each independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ or —C(O)R⁶.

In one example, Z¹ is a 3-10 membered carbocyclyl substituted with, for example, one or more of halogen and one or more of —OH. Similarly, in one example, Z¹ is a 3-10 membered carbocyclyl substituted with, for example, one or more of halogen and one or more of —OH. Similarly, in one example, Z¹ is a 3-10 membered carbocyclyl substituted with, for example, one or more of halogen and one or more of —OH and one or more of —C₁₋₆alkyl.

In one example, Z¹ is a 3-10 membered heterocyclyl substituted with, for example, one or more of halogen and one or more of —OH. Similarly, in one example, Z¹ is a 3-10 membered heterocyclyl substituted with, for example, one or more of halogen and one or more of —OH. Similarly, in one example, Z¹ is a 3-10 membered heterocyclyl substituted with, for example, one or more of halogen and one or more of —OH and one or more of —C₁₋₆alkyl.

In one example, Z² is a 3-10 membered carbocyclyl substituted with, for example, one or more of halogen and one or more of —OH. Similarly, in one example, Z² is a 3-10 membered carbocyclyl substituted with, for example, one or more of halogen and one or more of —OH. Similarly, in one example, Z² is a 3-10 membered carbocyclyl substituted with, for example, one or more of halogen and one or more of —OH and one or more of —C₁₋₆alkyl.

In one example, Z² is a 3-10 membered heterocyclyl substituted with, for example, one or more of halogen and one or more of —OH. Similarly, in one example, Z² is a 3-10 membered heterocyclyl substituted with, for example, one or more of halogen and one or more of —OH. Similarly, in one example, Z² is a 3-10 membered heterocyclyl substituted with, for example, one or more of halogen and one or more of —OH and one or more of —C₁₋₆alkyl.

In one example, Z³ is a 3-10 membered carbocyclyl substituted with, for example, one or more of halogen and one or more of —OH. Similarly, in one example, Z³ is a 3-10 membered carbocyclyl substituted with, for example, one or more of halogen and one or more of —OH. Similarly, in one example, Z³ is a 3-10 membered carbocyclyl substituted with, for example, one or more of halogen and one or more of —OH and one or more of —C₁₋₆alkyl.

In one example, Z³ is a 3-10 membered heterocyclyl substituted with, for example, one or more of halogen and one or more of —OH. Similarly, in one example, Z³ is a 3-10 membered heterocyclyl substituted with, for example, one or more of halogen and one or more of —OH. Similarly, in one example, Z³ is a 3-10 membered heterocyclyl substituted with, for example, one or more of halogen and one or more of —OH and one or more of —C₁₋₆alkyl.

It is to be understood that Z¹, Z² and Z³ are independent of one another. For example, in a compound of Formula (I), if present Z¹ may be a 3-10 membered heterocyclyl substituted with two halogens, if present Z² may be a 3-10 membered carbocyclyl substituted with one halogen and one —C₁₋₆alkyl, and if present Z³ may be a 3-10 membered carbocyclyl substituted with two halogens and one —C₁₋₆alkyl and one —NH₂.

In one example, Z¹, Z², and Z³ are each independently selected from the group consisting of:

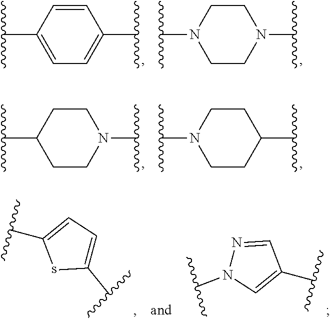

wherein each are unsubstituted or substituted with one or more substituents inde pendently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z¹ is selected from the group consisting of:

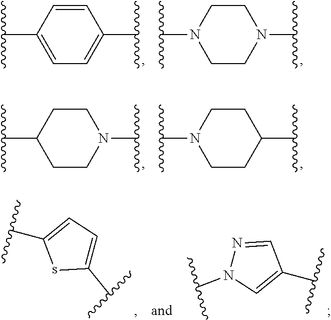

wherein each are unsubstituted or substituted with one or more substituents inde pendently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z² is selected from the group consisting of:

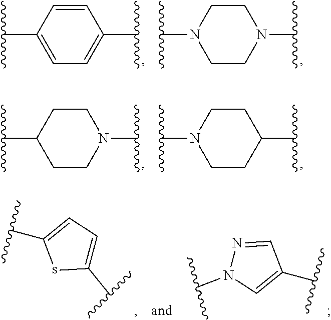

wherein each are unsubstituted or substituted with one or more substituents inde pendently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, Z³ is selected from the group consisting of:

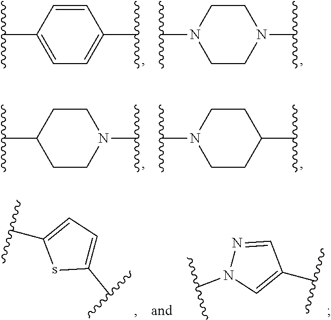

wherein each are unsubstituted or substituted with one or more substituents inde pendently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶.

In one example, R¹¹ are each independently selected from the group consisting of hydrogen, hydroxy, —C(O)OC₁₋₆alkyl, —C(O)C₁₋₆alkyl, —C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁-6alkyl), —C(O)N(C₁₋₆alkyl)₂, —C(O)OH, —C(O)OC₁₋₆alkyl, and —C(O)C₁₋₆alkyl. In one example, the C₁₋₆alkyl is selected from the group consisting of —CH₃, —CH₂CH₃, —CH₂CH₂CH₃, —CH₂CH₂CH₂CH₃, —CH₂CH₂CH₂CH₂CH₃, and —CH₂CH₂CH₂CH₂CH₂CH₃.

Formula (II) may be more specifically described by Formula (IIa). In one example, A is a linker of Formula (IIa):

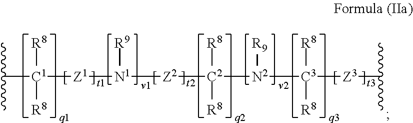

wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and if present, R⁶ and R⁷ are each independently selected from the group consisting of hydrogen, —C₁₋₆alkyl, 3-10 membered carbocyclyl, and 3-10 membered heterocyclyl.

In one example, q¹ is 0, 1, 2, 3, 4, or 5. In one example, q¹ is 0, and it will be understood that no C¹ is present in the linker of Formula (Ha). Similarly, in one example, q¹ is 1, and —C¹(R⁸)₂— is present in the linker. Similarly, in one example, q¹ is 2, and —C¹(R⁸)₂—C¹(R⁸)₂— is present in the linker. Similarly, in one example, q¹ is 3, and —C¹(R⁸)₂—C¹(R⁸)₂—C¹(R⁸)₂— is present in the linker. Similarly, in one example, q¹ is 4, and —C¹(R⁸)₂—C¹(R⁸)₂—C¹(R⁸)₂—C¹(R⁸)₂— is present in the linker. Similarly, in one example, q¹ is 5, and —C¹(R⁸)₂—C¹(R⁸)₂—C¹(R⁸)₂—C¹(R⁸)₂—C¹(R⁸)₂— is present in the linker.

In one example, q² is 0, 1, 2, 3, 4, or 5. In one example, q² is 0, and it will be understood that no C² is present in the linker of Formula (IIa). Similarly, in one example, q² is 1, and —C²(R⁸)₂— is present in the linker. Similarly, in one example, q² is 2, and —C²(R⁸)₂—C²(R⁸)₂— is present in the linker. Similarly, in one example, q² is 3, and —C²(R⁸)₂—C²(R⁸)₂—C²(R⁸)₂— is present in the linker. Similarly, in one example, q² is 4, and —C²(R⁸)₂—C²(R⁸)₂—C²(R⁸)₂—C²(R⁸)₂— is present in the linker. Similarly, in one example, q² is 5, and —C²(R⁸)₂—C²(R⁸)₂—C²(R⁸)₂—C²(R⁸)₂—C²(R⁸)₂— is present in the linker.

In one example, q³ is 0, 1, 2, 3, 4, or 5. In one example, q³ is 0, and it will be understood that no C³ is present in the linker of Formula (IIa). Similarly, in one example, q³ is 1, and —C³(R⁸)₂— is present in the linker. Similarly, in one example, q³ is 2, and —C³(R⁸)₂—C³(R⁸)₂— is present in the linker. Similarly, in one example, q³ is 3, and —C³(R⁸)₂—C³(R⁸)₂—C³(R⁸)₂— is present in the linker. Similarly, in one example, q³ is 4, and —C³(R⁸)₂—C³(R⁸)₂—C³(R⁸)₂—C³(R⁸)₂— is present in the linker. Similarly, in one example, q³ is 5, and —C³(R⁸)₂—C³(R⁸)₂—C³(R⁸)—C³(R⁸)₂—C³(R⁸)₂— is present in the linker.

It will be appreciated that when, for example, q¹ is 2 and —C¹(R⁸)₂—C¹(R⁸)₂— is present in the linker, each C¹(R⁸)₂ is independently selected. That is, the one or more C¹(R⁸)₂ present in the linker are independent of one another.

In one example, t¹ is 0, 1, or 2. In one example, t¹ is 0, and it will be understood that no Z¹ is present in the linker of Formula (Ha). Similarly, in one example, t¹ is 1, and —Z¹— is present in the linker. Similarly, in one example, t¹ is 2, and —Z¹—Z¹— is present in the linker.

In one example, t² is 0, 1, or 2. In one example, t² is 0, and it will be understood that no Z² is present in the linker of Formula (Ha). Similarly, in one example, t² is 1, and —Z²— is present in the linker. Similarly, in one example, t² is 2, and —Z²—Z²— is present in the linker.

In one example, t³ is 0, 1, or 2. In one example, t³ is 0, and it will be understood that no Z³ is present in the linker of Formula (Ha). Similarly, in one example, t³ is 1, and —Z³— is present in the linker. Similarly, in one example, t³ is 2, and —Z³—Z³— is present in the linker.

It will be appreciated that when, for example, t¹ is 2 and —Z¹—Z¹— is present in the linker, each Z¹ is independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ or —C(O)R⁶. In one example, t¹ is 2 and —Z¹—Z¹— is present in the linker as -3-10 membered carbocyclyl-3-10 membered heterocyclyl-. That is, the one or more Z¹ present in the linker are independent of one another.

In one example, v¹ is 0, 1, or 2. In one example, v¹ is 0, and it will be understood that no N¹(R⁹) is present in the linker of Formula (IIa). Similarly, in one example, v¹ is 1, and —N¹(R⁹)— is present in the linker. Similarly, in one example, v¹ is 2, and —N¹(R⁹)— N¹(R⁹)— is pre sent in the linker.

In one example, v² is 0, 1, or 2. In one example, v² is 0, and it will be understood that no N²(R⁹) is present in the linker of Formula (IIa). Similarly, in one example, v² is 1, and —N²(R⁹)— is present in the linker. Similarly, in one example, v² is 2, and —N²(R⁹)—N²(R⁹)— is present in the linker.

In one example, v³ is 0, 1, or 2. In one example, v³ is 0, and it will be understood that no N³(R⁹) is present in the linker of Formula (IIa). Similarly, in one example, v³ is 1, and —N³(R⁹)— is present in the linker. Similarly, in one example, v³ is 2, and —N³(R⁹)—N³(R⁹)— is present in the linker.

In one example, at least one of q¹, q², q³, t¹, t², t³, v¹, and v², is at least 1. That is, the linker of Formula (IIa) is present in Formula (I) (i.e., there is not direct attachment of the left-hand side to the right-hand side). That is, in one example, A is present in Formula (I).

In one example, each R⁸ is independently selected from the group consisting of hydro gen, —OH, —C₁₋₆alkyl, —C(O)OC₁₋₆alkyl, and —C(O)C₁₋₆alkyl; or two R⁸ are taken together form a C═O substituent with the adjacent carbon atom; wherein the —C₁₋₆alkyl is unsubstituted or sub stituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶; and wherein R⁶ and R⁷ are each independently selected from the group consisting of hydrogen, —C₁₋₆alkyl, 3-10 membered carbocyclyl, and 3-10 membered heterocyclyl.

In one example, R⁸ is hydrogen. In one example, R⁸ is —OH. In one example, R⁸ is —C₁₋₆alkyl. In one example, R⁸ is —C(O)OC₁₋₆alkyl. In one example, R⁸ is —C(O)C₁₋₆alkyl. In one example, two R⁸ are taken together form a C═O substituent with the adjacent carbon atom.

In one example, each R⁹ is independently selected from the group consisting of hydro gen, —OH, —C₁₋₆alkyl, —C(O)OC₁₋₆alkyl, and —C(O)C₁₋₆alkyl; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶; and wherein R⁶ and R⁷ are each independently selected from the group consisting of hydrogen, —C₁₋₆alkyl, 3-10 membered carbocyclyl, and 3-10 membered heterocyclyl.

In one example, R⁹ is hydrogen. In one example, R⁹ is —OH. In one example, R⁹ is —C₁₋₆alkyl. In one example, R⁹ is —C(O)OC₁₋₆alkyl. In one example, R⁹ is —C(O)C₁₋₆alkyl. In one example, R⁹ is C(O)CH₃. In one example, R⁹ is C(O)OC(CH₃)₃.

In one example, Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶; wherein if present, R⁶ and R⁷ are each independently selected from the group consisting of hydrogen, —C₁₋₆alkyl, 3-10 membered carbocyclyl, and 3-10 membered heterocyclyl.

In one example, t¹ is 0, 1, or 2. In one example, t¹ is 0, and it will be understood that no Z¹ is present in the linker of Formula (II). Similarly, in one example, t¹ is 1, and —Z¹— is present in the linker. Similarly, in one example, t¹ is 2, and —Z¹—Z¹— is present in the linker.

In one example, t² is 0, 1, or 2. In one example, t² is 0, and it will be understood that no Z² is present in the linker of Formula (II). Similarly, in one example, t² is 1, and —Z²— is present in the linker. Similarly, in one example, t² is 2, and —Z²—Z²— is present in the linker.

In one example, t³ is 0, 1, or 2. In one example, t³ is 0, and it will be understood that no Z³ is present in the linker of Formula (II). Similarly, in one example, t³ is 1, and —Z³— is present in the linker. Similarly, in one example, t³ is 2, and —Z³—Z³— is present in the linker.

It will be appreciated that when, for example, t¹ is 2 and —Z¹—Z¹— is present in the linker, each Z¹ is independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶. In one example, t¹ is 2 and —Z¹—Z¹— is present in the linker as -3-10 membered carbocyclyl-3-10 membered heterocyclyl. That is, the one or more Z¹ present in the linker are independent of one another.

In one example, C¹, C², and C³ are each carbon atoms. In one example, N¹ and N² are each nitrogen atoms.

In one example, Formula (IIa) is selected from the group consisting of:

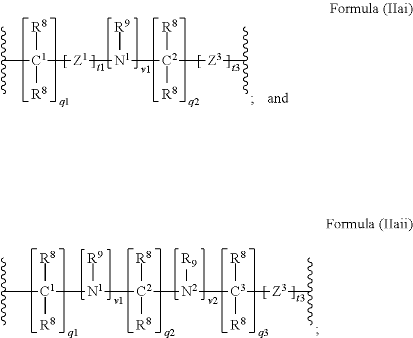

wherein R⁸ and R⁹ are as described herein; C¹, C², and C³ are each carbon; N¹ and N² are each nitrogen; Z¹ and Z³ are as described herein; q¹, q², and q³ are each independently 0, 1, 2, 3, 4, or 5; t¹ and t³ are each independently 0, 1, or 2; v¹ and v² are each independently 0, 1 or 2; and wherein at least one of q¹, q², q³, t¹, t², t³, v¹, and v², is at least 1.

In one example, Formula (IIa) is selected from the group consisting of:

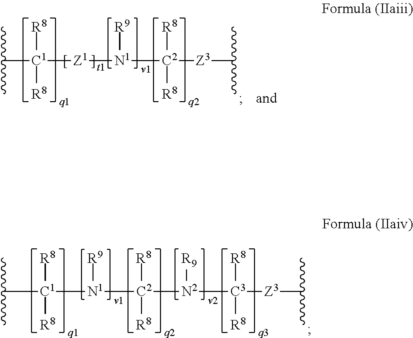

wherein R⁸ and R⁹ are as described herein; C¹, C², and C³ are each carbon; N¹ and N² are each nitrogen; Z¹ and Z³ are as described herein; q¹, q², and q³ are each independently 0, 1, 2, 3, 4, or 5; t¹ is 0, 1, or 2; and v¹ and v² are each independently 0, 1 or 2.

In one example, wherein t¹ is 1, and Z¹ is Formula (III):

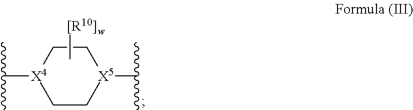

wherein X⁴ is CH or N; X⁵ is CH or N; R¹⁰ are each independently selected from the group consisting of halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —CN, —OR⁶, —SR⁶, —N(R⁶)(R⁷), —C(O)R⁶, —C(O)OR⁶, —C(O)N(R⁶)(R⁷), —N(R⁶)C(O)R⁷, —S(O)OR⁶, —S(O)ON(R⁶)(R⁷), and —N(R⁶)S(O)OR⁷; w is 0, 1, 2, 3, 4, 5, 6, 7, or 8; and if present, R⁶ and R⁷ are as described herein.

In one example, X⁴ is CH or N. In one example X⁴ is CH. In one example, X⁴ is N.

In one example, X⁵ is CH or N. In one example X⁵ is CH. In one example, X⁵ is N.

In one example, X⁴ is CH and X⁵ is CH. In one example, X⁴ is CH and X⁵ is N. In one example, X⁴ is N and X⁵ is CH. In one example, X⁴ is N and X⁵ is N.

In one example, R¹⁰ are each independently selected from the group consisting of halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —CN, —OR⁶, —SR⁶, —N(R⁶)(R⁷), —C(O)R⁶, —C(O)OR⁶, —C(O)N(R⁶)(R⁷), —N(R⁶)C(O)R⁷, —S(O)OR⁶, —S(O)ON(R⁶)(R⁷), and —N(R⁶)S(O)OR⁷.

In one example, R¹⁰ is halogen. In one example, R¹⁰ is —C₁₋₆alkyl.

In one example, w of Formula (III) is 0, 1, 2, 3, 4, 5, 6, 7, or 8. In Formula (III), it will be appreciated that w designates the number of R¹⁰ substituents. In one example, w of Formula (III) is 0. In the instance where w of Formula (III) is 0, it will be understood that the R¹⁰ substituent is not present in Formula (III). In one example, w of Formula (III) is 1, and one R¹⁰ substituent is present in Formula (III). In one example, w of Formula (III) is 2, and two R¹⁰ substituents are present in Formula (III). In one example, w of Formula (III) is 3, and three R¹⁰ substituents are present in Formula (III). In one example, w of Formula (III) is 4, and four R¹⁰ substituents are present in Formula (III). In one example, w of Formula (III) is 5, and five R¹⁰ substituents are present in Formula (III). In one example, w of Formula (III) is 6, and six R¹⁰ substituents are present in Formula (III). In one example, w of Formula (III) is 7, and seven R¹⁰ substituents are present in Formula (III). In one example, w of Formula (III) is 8, and eight R¹⁰ substituents are present in Formula (III).

In one example, w is 1, 2, 3, 4, 5, 6, 7, or 8, and the R¹⁰ substituent(s) is be attached to any carbon, and in any configuration, on the designated ring of Formula (III).

In one example, when w is 2, the two R¹⁰ substituents present in Formula (III), may be attached to the same or different carbon atoms of the designated ring:

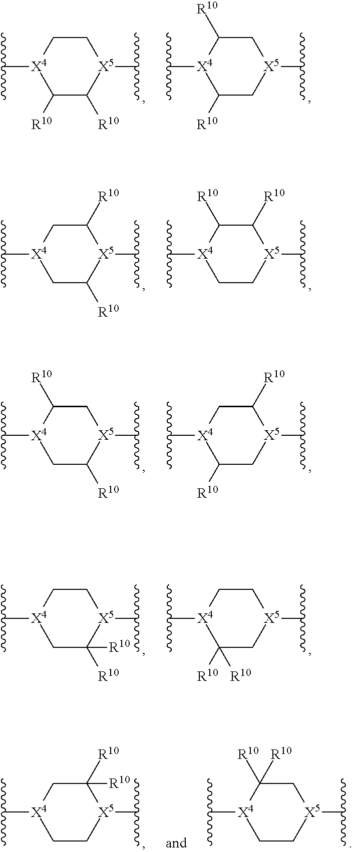

The same will be understood when w is 3, 4, 5, 6, 7, or 8.

In one example, Formula (III) is selected from the group consisting of:

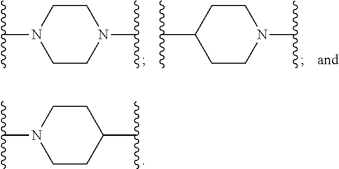

In one example, Formula (IIa) is selected from the group consisting of:

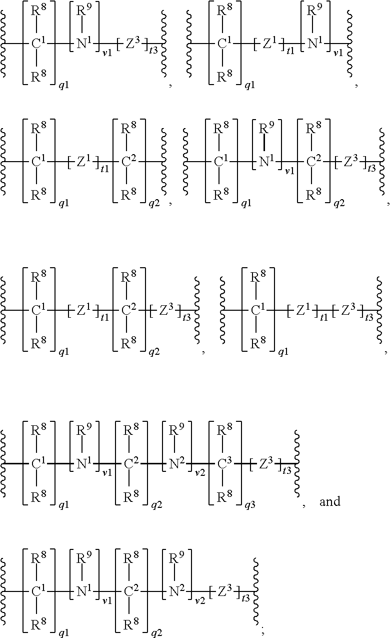

wherein, if present, R⁸ and R⁹ are as described herein; if present, C¹, C², and C³ are each carbon; if present, N¹ and N² are each nitrogen; if present, Z^{t}, Z², and Z³ are as described herein; if present, q^{t}, q², and q³ are each independently 0, 1, 2, 3, 4, or 5; if present, t¹, t², and t³ are each independently 0, 1, or 2; if present, v¹ and v² are each independently 0, 1 or 2; and wherein at least one of q¹, q², q³, t¹, t², t³, v¹, and v², is at least 1.

In one example, Formula (IIa) is selected from the group consisting of:

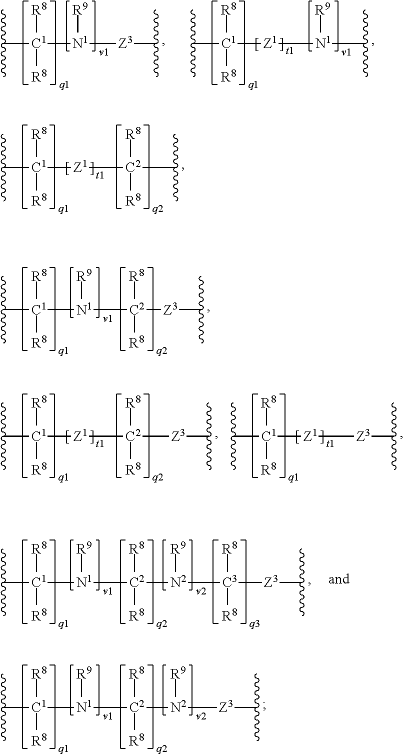

wherein, if present, R⁸ and R⁹ are as described herein; if present, C¹, C², and C³ are each carbon; if present, N^{t} and N² are each nitrogen; if present, Z¹, Z², and Z³ are as described herein; if present, q^{t}, q², and q³ are each independently 0, 1, 2, 3, 4, or 5; if present, t¹ is 0, 1, or 2; and if present, v¹ and v² are each independently 0, 1 or 2.

In one example, Formula (IIa) is selected from the group consisting of:

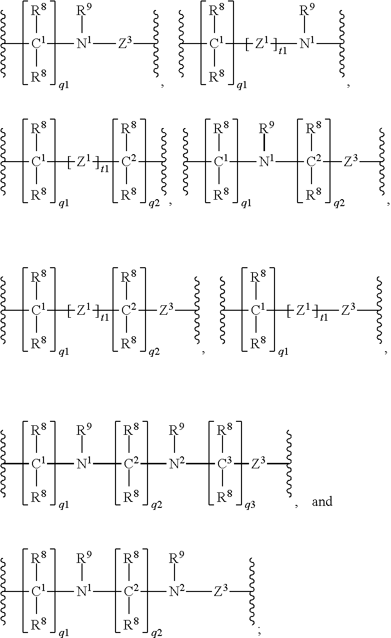

wherein, if present, R⁸ and R⁹ are as described herein; if present, C¹, C², and C³ are each carbon; if present, N^{t} and N² are each nitrogen; if present, Z¹, Z², and Z³ are as described herein; and if present, q^{t}, q², and q³ are each independently 0, 1, 2, 3, 4, or 5.

In one example, Formula (IIa) is selected from the group consisting of:

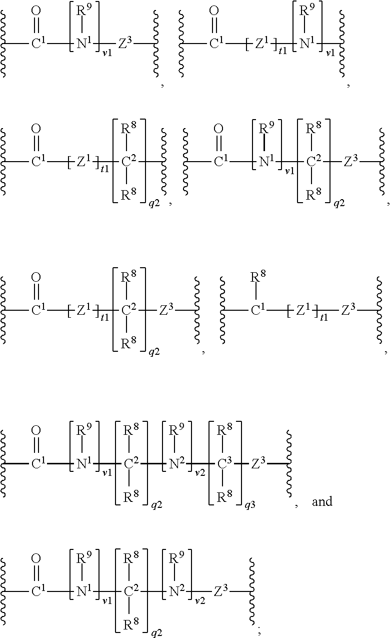

wherein, if present, R⁸ and R⁹ are as described herein; if present, C¹, C², and C³ are each carbon; if present, N¹ and N² are each nitrogen; if present, Z¹, Z², and Z³ are as described herein; if present, q² and q³ are each independently 0, 1, 2, 3, 4, or 5; if present, t¹ is 0, 1, or 2; and if present, v¹ and v² are each independently 0, 1 or 2.

In one example, Formula (IIa) is selected from the group consisting of:

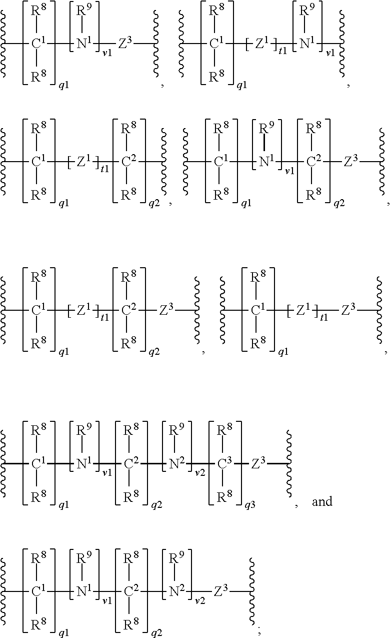

wherein, if present, R⁸ and R⁹ are as defined herein; if present, C¹, C², and C³ are each carbon; if present, N¹ and N² are each nitrogen; if present, Z¹ and Z² are as described herein; and Z³ is selected from the group consisting of:

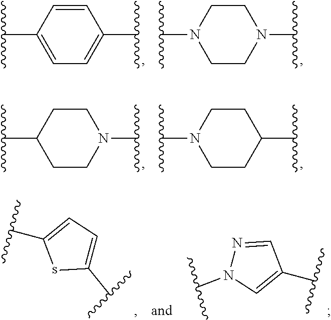

if present, q¹, q², and q³ are each independently 0, 1, 2, 3, 4, or 5; if present, t¹ is 0, 1, or 2; and if present, v¹ and v² are each independently 0, 1 or 2.

In one example, Formula (IIa) is selected from the group consisting of:

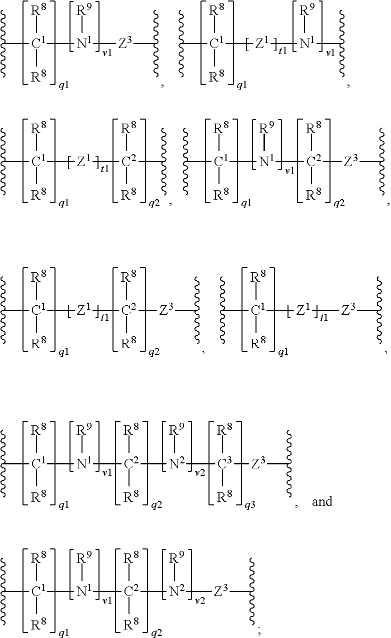

wherein, if present, R⁸ and R⁹ are as described herein; if present, C¹, C², and C³ are each carbon; if present, N¹ and N² are each nitrogen; if present, Z¹ and Z² are as described herein; Z³ is:

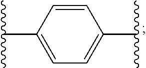 [SMILES: *c1ccc(*)cc1]

if present, q¹, q², and q³ are each independently 0, 1, 2, 3, 4, or 5; if present, t¹ is 0, 1, or 2; and if present, v¹ and v² are each independently 0, 1 or 2.

In one example, A, or Formula (II), or Formula (IIa) is selected from the group consisting of:

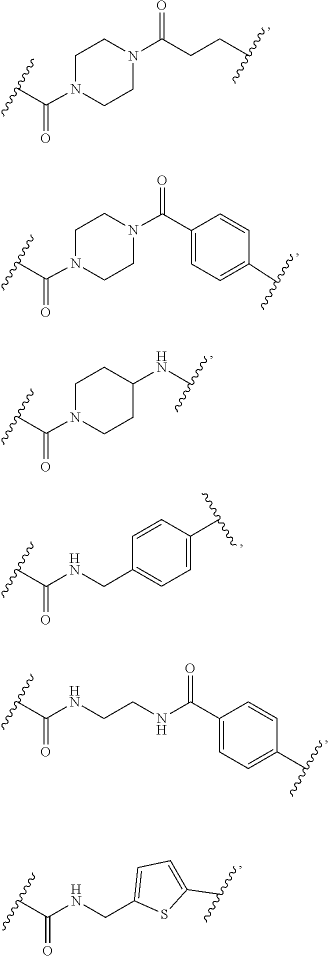 [SMILES: *CCC(=O)N1CCN(C(*)=O)CC1 ; *C(=O)N1CCN(C(=O)c2ccc(*)cc2)CC1 ; *NC1CCN(C(*)=O)CC1 ; *C(=O)NCc1ccc(*)cc1 ; *C(=O)NCCNC(=O)c1ccc(*)cc1 ; *C(=O)NCc1ccc(*)s1]

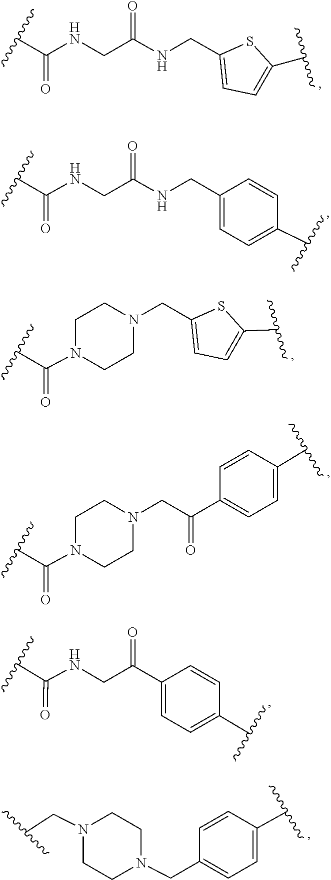 [SMILES: *C(=O)NCC(=O)NCc1ccc(*)s1 ; *C(=O)NCC(=O)NCc1ccc(*)cc1 ; *C(=O)N1CCN(Cc2ccc(*)s2)CC1 ; *C(=O)N1CCN(CC(=O)c2ccc(*)cc2)CC1 ; *C(=O)NCC(=O)c1ccc(*)cc1 ; *CN1CCN(Cc2ccc(*)cc2)CC1]

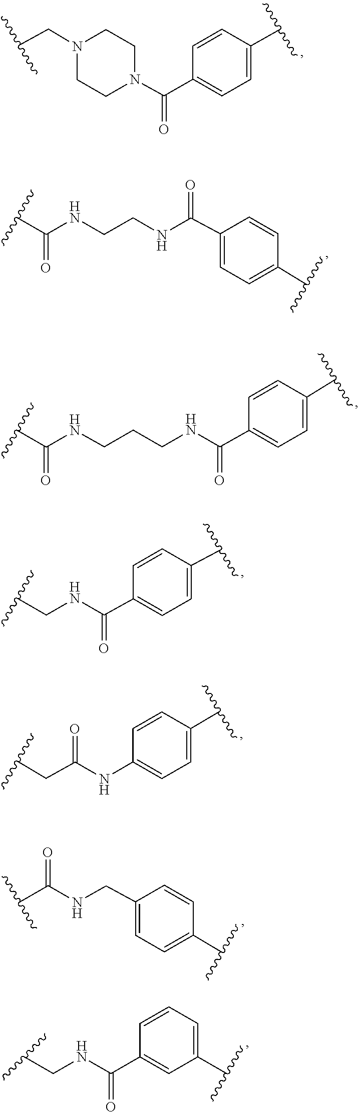

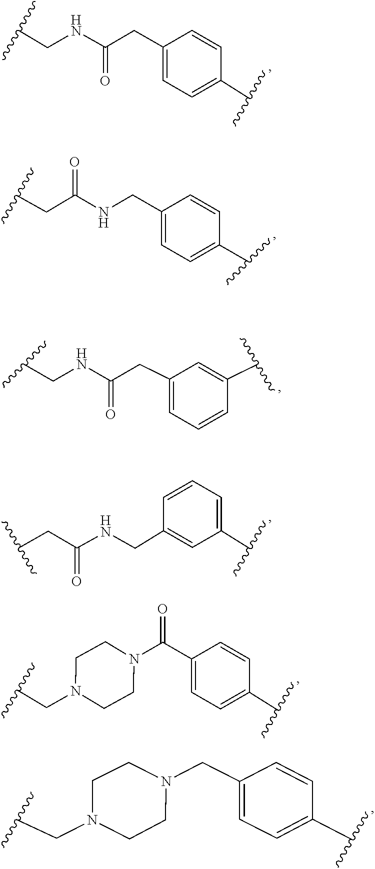 [SMILES: *CNC(=O)Cc1ccc(*)cc1 ; *CC(=O)NCc1ccc(*)cc1 ; *CNC(=O)Cc1cccc(*)c1 ; *CC(=O)NCc1cccc(*)c1 ; *CN1CCN(C(=O)c2ccc(*)cc2)CC1 ; *CN1CCN(Cc2ccc(*)cc2)CC1]

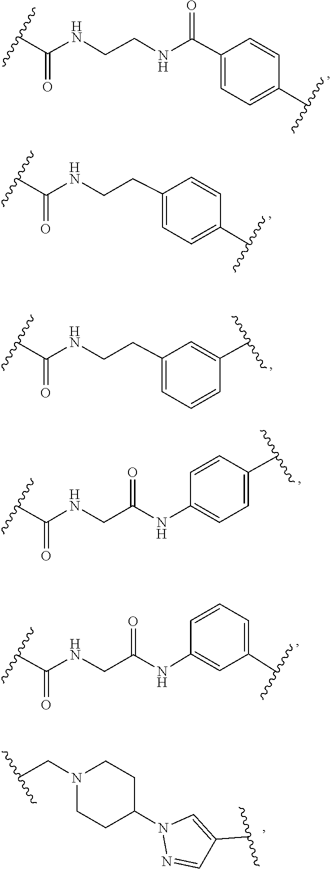 [SMILES: *C(=O)NCCNC(=O)c1ccc(*)cc1 ; *C(=O)NCCc1ccc(*)cc1 ; *C(=O)NCCc1cccc(*)c1 ; *C(=O)NCC(=O)Nc1ccc(*)cc1 ; *C(=O)NCC(=O)Nc1cccc(*)c1 ; *CN1CCC(n2cc(*)cn2)CC1]

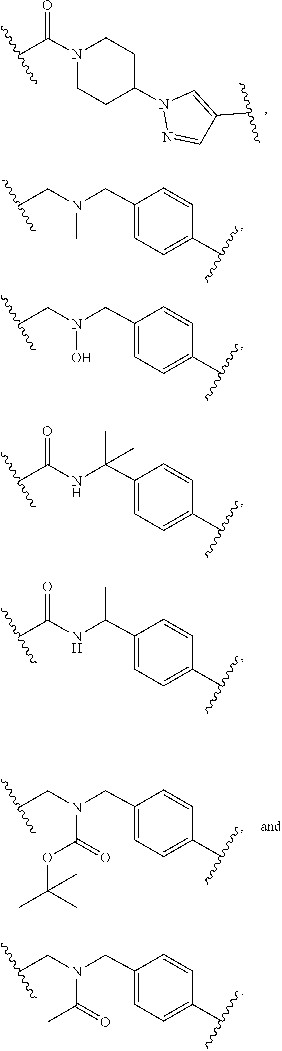

In one example, A, or Formula (II), or Formula (IIa) is:

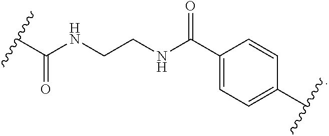 [SMILES: *C(=O)NCCNC(=O)c1ccc(*)cc1]

In one example, A, or Formula (II), or Formula (IIa) is:

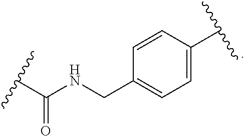 [SMILES: *C(=O)NCc1ccc(*)cc1]

In one example, A is a linker selected to provide a shortest pathway of covalently linked atoms of between about 3 to 30 atoms, 4 to 20 atoms 5 to 15 atoms, or 6 to 12 atoms. The atoms of the linker may be selected from C, O, N, and S. As described herein, the atoms of the linker may be provided by any one or more of the groups A¹, A², A³, Z¹, Z², and Z³.

### Compounds of Formula (I)

It will be appreciated that the compound of Formula (I) may be any suitable combination of left-hand side of the compound, linker, and right-hand side of the compound, as described herein.

In one example, the compound of Formula (I) is:

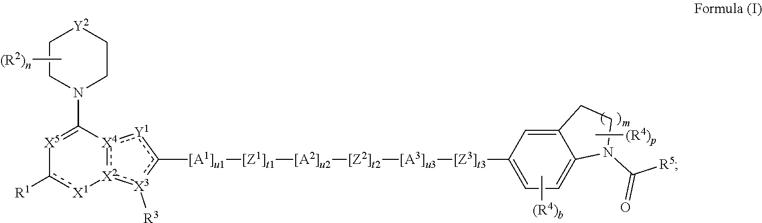

wherein
   X¹ is C(R) or N;
   X² is CH, C or N;
   X³ is C or N;
   X⁴ is C or N;
   X⁵ is C or N;
   Y¹ is CH, N or S;
   Y² is O, CH₂, or N(R);
   R is selected from the group consisting of hydrogen, halogen, and C₁₋₆alkyl;
   R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
      wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
         wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
   R⁵ is selected from the group consisting of —C₁₋₆alkyl, —O(R⁶), 3-10 membered carbocyclyl, and 3-10 membered heterocyclyl;
   m is 1, 2, or 3;
   n is 0, 1, 2, 3, 4, 5, 6, 7, or 8;
   p is 0, 1, 2, 3, 4, 5, 6, 7, or 8;
   b is 0, 1, 2, or 3;
   A¹, A², and A³ are each independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶;
   Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
   u¹, u², and u³ are each independently 0, 1, 2, 3, 4, or 5;
   t¹, t², and t³ are each independently 0, 1, or 2; and
   wherein at least one of u¹, u², u³, t¹, t², and t³, is at least 1; and
   if present, R⁶ and R⁷ are each independently selected from the group consisting of hydrogen, —C₁₋₆alkyl, 3-10 membered carbocyclyl, and 3-10 membered heterocyclyl; and
   if present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

In one example, the compound of Formula (I) is:

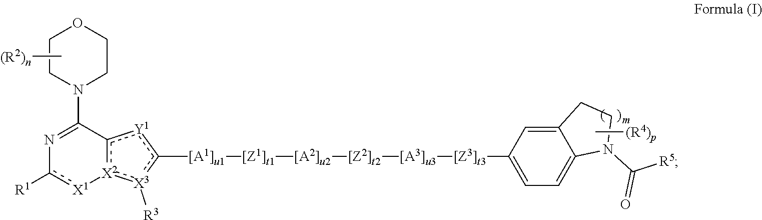

wherein
X¹ is C(R) or N;
X² is CH, C or N;
X³ is C or N;
Y¹ is CH, N or S;
R is selected from the group consisting of hydrogen, halogen, and C₁₋₆alkyl;
R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
   wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
      wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
m is 1, 2, or 3;
n is 0, 1, 2, 3, 4, 5, 6, 7, or 8;
p is 0, 1, 2, 3, 4, 5, 6, 7, or 8;
A¹, A², and A³ are each independently selected from the group consisting of —C₁₋₆alkylene-, —C(O)—, —C(O)N(R⁶)—, —N(R⁶)C(O)—, —N(R⁶)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
u¹, u², and u³ are each independently 0, 1, 2, 3, 4, or 5;
t¹, t², and t³ are each independently 0, 1, or 2; and
wherein at least one of u¹, u², u³, t¹, t², and t³, is at least 1; and
if present, R⁶ and R⁷ are each independently selected from the group consisting of hydrogen, —C₁₋₆alkyl, —C(O)OC₁₋₆alkyl, —C(O)C₁₋₆alkyl, 3-10 membered carbocyclyl, and 3-10 membered heterocyclyl.

In one example, the compound of Formula (I) is:

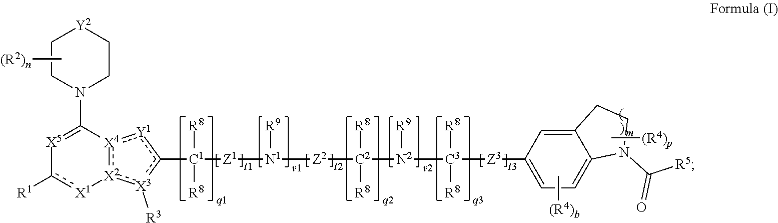

wherein X¹, X², X³, X⁴, X⁵, Y¹, Y², R¹, R², R³, R⁴, R⁵, m, n, p, b, R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is:

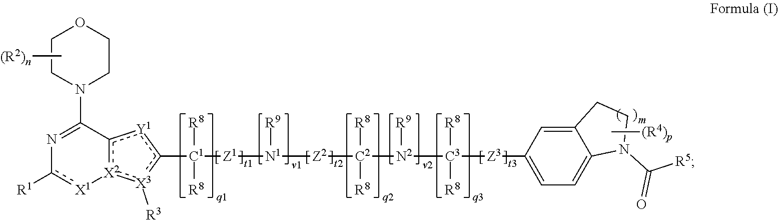

wherein X¹, X², X³, Y¹, R¹, R², R³, R⁴, R⁵, m, n, p, R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v^{t} and v² are each independently 0, 1 or 2; and wherein at least one of q¹, q², q³, t¹, t², t³, v¹, and v², is at least 1.

It will be appreciated that any one or more of the embodiments or examples as described herein, for the variables X¹, X², X³, X⁴, X⁵, Y¹, Y², R¹, R², R³, R⁴, R⁵, m, n, p, b, A, A¹, A², A³, Z¹, Z², Z³, u¹, u², u³, t¹, t², t³, R⁶, R⁷, R⁸, R⁹, q¹, q², q³, t¹, t², t³, v¹ and v², either taken separately or together may be provided for the above compounds of Formula (I).

In one example, the compound of Formula (I) is:

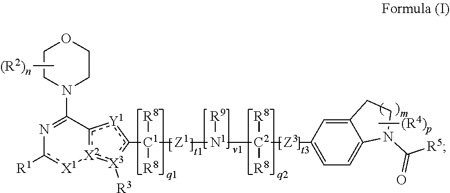

wherein X¹, X², X³, Y¹, R¹, R², R³, R⁴, R⁵, m, n, p, R⁸, R⁹, C¹, C², N¹, Z¹, and Z³ are as described herein; q¹ and q² are each independently 0, 1, 2, 3, 4, or 5; t¹ and t³ are each inde pendently 0, 1, or 2; v¹ is 0, 1 or 2; and wherein at least one of q¹, q², t¹, t³, and v¹ is at least 1.

In one example, the compound of Formula (I) is:

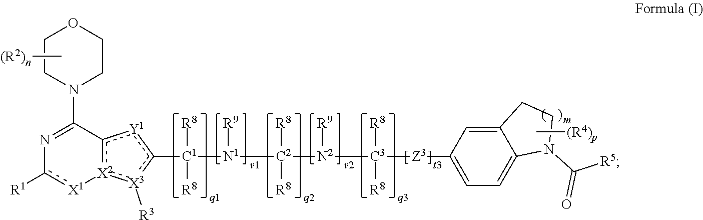

wherein X¹, X², X³, Y¹, R¹, R², R³, R⁴, R⁵, m, n, p, R⁸, R⁹, C¹, C², N¹, and Z³ are as described herein; q¹, q², and q³ are each independently 0, 1, 2, 3, 4, or 5; t³ is 0, 1 or 2; v¹ and v² are each independently 0, 1 or 2; and wherein at least one of q¹, q², q³, t³, v¹, and v², is at least 1.

In one example, the compound of Formula (I) is:

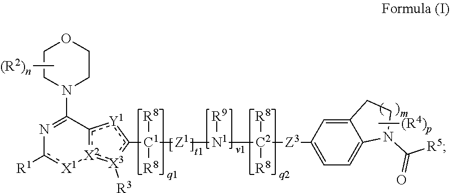

wherein X¹, X², X³, Y¹, R¹, R², R³, R⁴, R⁵, m, n, p, R⁸, R⁹, C¹, C², N¹, Z¹, and Z³ are as described herein; q¹ and q² are each independently 0, 1, 2, 3, 4, or 5; t¹ is 0, 1 or 2; v¹ is 0, 1 or 2; and wherein at least one of q¹, q², t¹, and v¹, is at least 1.

In one example, the compound of Formula (I) is:

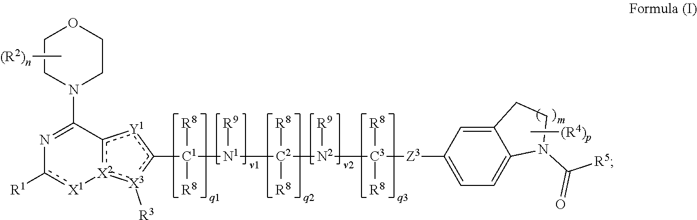

wherein X¹, X², X³, Y¹, R¹, R², R³, R⁴, R⁵, m, n, p, R⁸, R⁹, C¹, C², N¹, and Z³ are as described herein; q¹, q², and q³ are each independently 0, 1, 2, 3, 4, or 5; v¹ and v² are each independently 0, 1 or 2; and wherein at least one of q¹, q², q³, v¹, and v², is at least 1.

In one example, the compound of Formula (I) is:

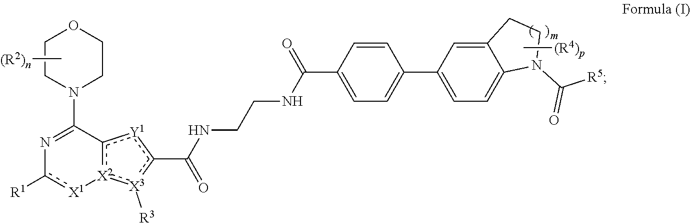

wherein X¹, X², X³, Y¹, R¹, R², R³, R⁴, R⁵, m, n, and p are as described herein; In one example, the compound of Formula (I) is:

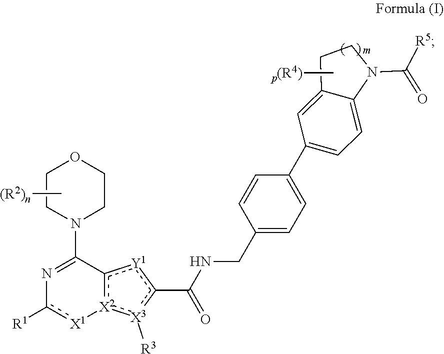

wherein X¹, X², X³, Y¹, R¹, R², R³, R⁴, R⁵ are as described herein; m is 1, 2, or 3; and n is 0, 1, 2, 3, 4, 5, 6, 7, or 8.

In one example, the compound of Formula (I) is a compound of Formula (Ia):

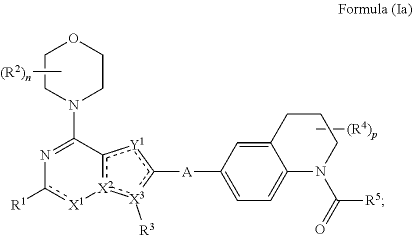

wherein X¹, X², X³, Y¹, R¹, R², R³, R⁴, R⁵, n, and p are as described herein;
A is a linker of Formula (IIa):

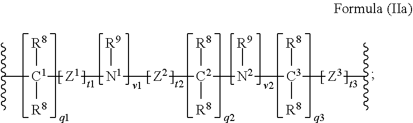

herein R⁸, R⁹, C¹, C², C³, N¹, N², Z¹, Z², and Z³ are as described herein; q¹, q², and q³ are each independently 0, 1, 2, 3, 4, or 5; t¹, t² and t³ are each independently 0, 1, or 2;
v¹ and v² are each independently 0, 1 or 2; and wherein at least one of q¹, q², q³, t¹, t², t³, v¹, and v², is at least 1.

In one example, the compound of Formula (I) is a compound of Formula (Ib):

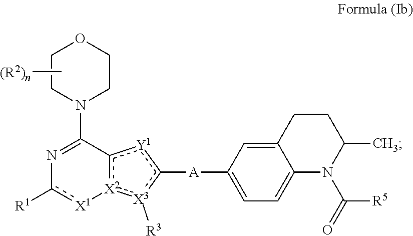

wherein X¹, X², X³, Y¹, R¹, R², R³, R⁴, R⁵, and n are as described herein;
A is a linker of Formula (IIa):

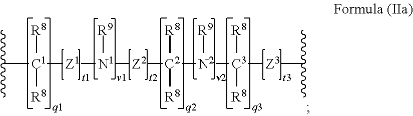

wherein R⁸, R⁹, C¹, C², C³, N¹, N², Z¹, Z², and Z³ are as described herein; q¹, q², and q³ are each independently 0, 1, 2, 3, 4, or 5; t¹, t² and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is a compound of Formula (Ic):

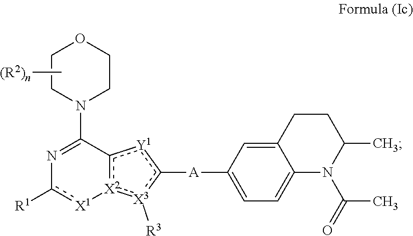

wherein X¹, X², X³, Y¹, R¹, R², R³, R⁴, and n are as described herein;
A is a linker of Formula (IIa):

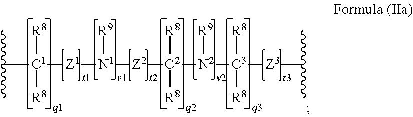

wherein R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is:

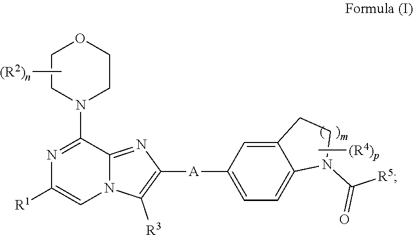

wherein R¹, R², R³, R⁴, R⁵, and n are as described herein; R⁵ is selected from the group consisting of —C₁₋₆alkyl, —O(R⁶), 3-10 membered carbocyclyl, and 3-10 membered heterocyclyl; m is 1, 2, or 3; n is 0, 1, 2, 3, 4, 5, 6, 7, or 8; p is 0, 1, 2, 3, 4, 5, 6, 7, or 8;
A is a linker of Formula (IIa):

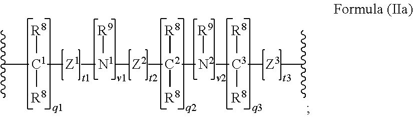

wherein R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is:

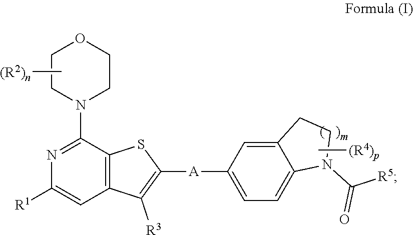

wherein R¹, R², R³, and R⁴ are as described herein; R⁵ is selected from the group consisting of —C₁₋₆alkyl, —O(R⁶), 3-10 membered carbocyclyl, and 3-10 membered heterocyclyl; m is 1, 2, or 3; n is 0, 1, 2, 3, 4, 5, 6, 7, or 8; p is 0, 1, 2, 3, 4, 5, 6, 7, or 8;
A is a linker of Formula (IIa):

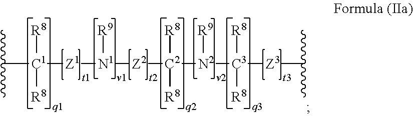

wherein R⁸, R⁹, C¹, C², C³, N¹, N², Z¹, Z², and Z³ are as described herein; q¹, q² and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is:

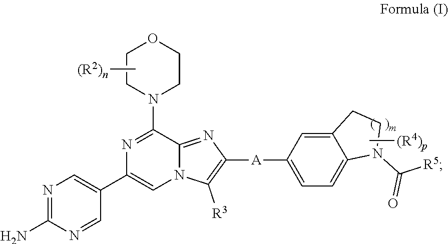

wherein R², R³, R⁴, R⁵, m, n, and p are as described herein;
A is a linker of Formula (IIa):

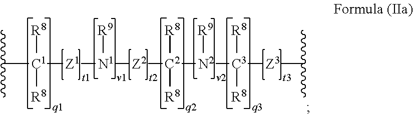

wherein R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is:

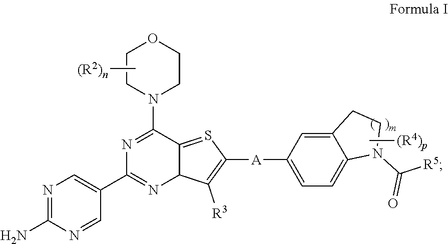

wherein R², R³, R⁴, R⁵, m, n and p are as described herein;
A is a linker of Formula (IIa):

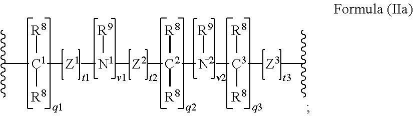

wherein R⁸, R⁹, C¹, C², C³, N¹, N², Z¹, Z², and Z³ are as described herein; q¹, q², and q³ are each independently 0, 1, 2, 3, 4, or 5; t¹, t², and t³ are each independently 0, 1, or 2; v¹ and v² are each independently 0, 1 or 2; and wherein at least one of q¹, q², q³, t¹, t², t³, v¹, and v², is at least 1.

In one example, the compound of Formula (I) is selected from the group consisting of:
1-(6-(4-(4-(6-(2-aminopyrimidin-5-yl)-8-morpholinoimidazo[1,2-a]pyrazine-2-carbonyl)pi-perazine-1-carbonyl)phenyl)-2-methyl-3,4-dihydroquinolin-1(2H)-yl)ethan-1-one;
N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
N-(2-((4-(1-acetyl-2-methyl-1,2,3,4-tetrahydroquinolin-6-yl)benzyl)amino)-2-oxoethyl)-6-(2-aminopyrimidin-5-yl)-8-morpholinoimidazo[1,2-a]pyrazine-2-carboxamide;
1-(6-(5-((4-(6-(2-aminopyrimidin-5-yl)-8-morpholinoimidazo[1,2-a]pyrazine-2-carbonyl)pi-perazin-1-yl)methyl)thiophen-2-yl)-2-methyl-3,4-dihydroquinolin-1(2H)-yl)ethan-1-one;
N-((5-(1-acetyl-2-methyl-1,2,3,4-tetrahydroquinolin-6-yl)thiophen-2-yl)methyl)-6-(2-amino-pyrimidin-5-yl)-8-morpholinoimidazo[1,2-a]pyrazine-2-carboxamide;
N-(2-(((5-(1-acetyl-2-methyl-1,2,3,4-tetrahydroquinolin-6-yl)thiophen-2-yl)methyl)amino)-2-oxoethyl)-6-(2-aminopyrimidin-5-yl)-8-morpholinoimidazo[1,2-a]pyrazine-2-carboxamide;
1-(4-(1-acetyl-2-methyl-1,2,3,4-tetrahydroquinolin-6-yl)phenyl)-2-(4-(6-(2-aminopyrimidin-5-yl)-8-morpholinoimidazo[1,2-a]pyrazine-2-carbonyl)piperazin-1-yl)ethan-1-one;
N-(2-(4-(1-acetyl-2-methyl-1,2,3,4-tetrahydroquinolin-6-yl)phenyl)-2-oxoethyl)-6-(2-amino-pyrimidin-5-yl)-8-morpholinoimidazo[1,2-a]pyrazine-2-carboxamide;
N-(2-(4-(1-acetyl-2-methyl-1,2,3,4-tetrahydroquinolin-6-yl)benzamido)ethyl)-6-(2-aminopy-rimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(3-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(2-(4-(1-acetyl-2-methyl-1,2,3,4-tetrahydroquinolin-6-yl)benzamido)ethyl)-6-(2-amino-pyrimidin-5-yl)-8-morpholinoimidazo[1,2-a]pyrazine-2-carboxamide;
(S)-N-(3-(4-(1-acetyl-2-methyl-1,2,3,4-tetrahydroquinolin-6-yl)benzamido)propyl)-6-(2-ami-nopyrimidin-5-yl)-8-morpholinoimidazo[1,2-a]pyrazine-2-carboxamide;
(S)-1-(6-(4-(4-((6-(2-aminopyrimidin-5-yl)-8-morpholinoimidazo[1,2-a]pyrazin-2-yl)methyl)piperazine-1-carbonyl)phenyl)-2-methyl-3,4-dihydroquinolin-1(2H)-yl)ethan-1-one;
(S)-1-(6-(4-((4-((6-(2-aminopyrimidin-5-yl)-8-morpholinoimidazo[1,2-a]pyrazin-2-yl)methyl)piperazin-1-yl)methyl)phenyl)-2-methyl-3,4-dihydroquinolin-1(2H)-yl)ethan-1-one;
(S)-4-(1-acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide;
(S)-3-(1-acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide;
(S)-2-(4-(1-acetyl-2-methyl-1,2,3,4-tetrahydroquinolin-6-yl)phenyl)-N-((6-(2-aminopyrim-idin-5-yl)-8-morpholinoimidazo[1,2-a]pyrazin-2-yl)methyl)acetamide;
(S)-2-(3-(1-acetyl-2-methyl-1,2,3,4-tetrahydroquinolin-6-yl)phenyl)-N-((6-(2-aminopyrim-idin-5-yl)-8-morpholinoimidazo[1,2-a]pyrazin-2-yl)methyl)acetamide;
(S)-N-(4-(1-acetyl-2-methyl-1,2,3,4-tetrahydroquinolin-6-yl)benzyl)-6-(2-aminopyrimidin-5-yl)-3-methyl-8-morpholinoimidazo[1,2-a]pyrazine-2-carboxamide;
(S)-N-(2-(4-(1-acetyl-2-methyl-1,2,3,4-tetrahydroquinolin-6-yl)benzamido)ethyl)-6-(2-amino-pyrimidin-5-yl)-3-methyl-8-morpholinoimidazo[1,2-a]pyrazine-2-carboxamide;
(S)-N-(4-(1-acetyl-2-methyl-1,2,3,4-tetrahydroquinolin-6-yl)phenyl)-2-(6-(2-aminopyrimidin-5-yl)-8-morpholinoimidazo[1,2-a]pyrazin-2-yl)acetamide;
(S)-N-(3-(1-acetyl-2-methyl-1,2,3,4-tetrahydroquinolin-6-yl)phenyl)-2-(6-(2-aminopyrimidin-5-yl)-8-morpholinoimidazo[1,2-a]pyrazin-2-yl)acetamide;
(S)-N-(4-(1-acetyl-2-methyl-1,2,3,4-tetrahydroquinolin-6-yl)benzyl)-2-(6-(2-aminopyrimidin-5-yl)-8-morpholinoimidazo[1,2-a]pyrazin-2-yl)acetamide;
(S)-N-(3-(1-acetyl-2-methyl-1,2,3,4-tetrahydroquinolin-6-yl)benzyl)-2-(6-(2-aminopyrimidin-5-yl)-8-morpholinoimidazo[1,2-a]pyrazin-2-yl)acetamide;
(S)-4-(1-acetyl-2-methyl-1,2,3,4-tetrahydroquinolin-6-yl)-N-((2-(2-aminopyrimidin-5-yl)-4-morpholinothieno[3,2-d]pyrimidin-6-yl)methyl)benzamide;
(S)-2-(4-(1-acetyl-2-methyl-1,2,3,4-tetrahydroquinolin-6-yl)phenyl)-N-((2-(2-aminopyrim-idin-5-yl)-4-morpholinothieno[3,2-d]pyrimidin-6-yl)methyl)acetamide;
(S)-1-(6-(4-(4-((2-(2-aminopyrimidin-5-yl)-4-morpholinothieno[3,2-d]pyrimidin-6-yl)methyl)piperazine-1-carbonyl)phenyl)-2-methyl-3,4-dihydroquinolin-1(2H)-yl)ethan-1-one;
(S)-1-(6-(4-((4-((2-(2-aminopyrimidin-5-yl)-4-morpholinothieno[3,2-d]pyrimidin-6-yl)methyl)piperazin-1-yl)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)benzyl)-2-(2-aminopyrimidin-5-yl)-4-morpholinothieno[3,2-d]pyrimidine-6-carboxamide;
(S)-N-(2-(4-(1-acetyl-2-methyl-1,2,3,4-tetrahydroquinolin-6-yl)benzamido)ethyl)-2-(2-amino-pyrimidin-5-yl)-4-morpholinothieno[3,2-d]pyrimidine-6-carboxamide;
(S)-N-(4-(1-acetyl-2-methyl-1,2,3,4-tetrahydroquinolin-6-yl)benzyl)-2-(2-aminopyrimidin-5-yl)-7-methyl-4-morpholinothieno[3,2-d]pyrimidine-6-carboxamide;
(S)-N-(2-(4-(1-acetyl-2-methyl-1,2,3,4-tetrahydroquinolin-6-yl)benzamido)ethyl)-2-(2-amino-pyrimidin-5-yl)-7-methyl-4-morpholinothieno[3,2-d]pyrimidine-6-carboxamide;
(R)-N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-8-(piperazin-1-yl)imidazo[1,2-a]pyrazine-2-carboxamide hydrochloride;
(S)-N-(2-(4-(1-acetyl-2-methyl-1,2,3,4-tetrahydroquinolin-6-yl)-N-methylbenzamido)ethyl)-6-(2-aminopyrimidin-5-yl)-N-methyl-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-N-methyl-8-morpholinoimidazo[1,2-a]pyrazine-2-carboxamide;
(S)-N-(4-(1-acetyl-2-methyl-1,2,3,4-tetrahydroquinolin-6-yl)benzyl)-2-(2-aminopyrimidin-5-yl)-N-methyl-4-morpholinothieno[3,2-d]pyrimidine-6-carboxamide;
(S)-N-(4-(1-acetyl-2-methyl-1,2,3,4-tetrahydroquinolin-6-yl)benzyl)-2-(2-aminopyrimidin-5-yl)-N,7-dimethyl-4-morpholinothieno[3,2-d]pyrimidine-6-carboxamide;
(S)-N-(3-(4-(1-acetyl-2-methyl-1,2,3,4-tetrahydroquinolin-6-yl)benzamido)propyl)-2-(2-ami-nopyrimidin-5-yl)-4-morpholinothieno[3,2-d]pyrimidine-6-carboxamide;
(S)-N-(3-(4-(1-acetyl-2-methyl-1,2,3,4-tetrahydroquinolin-6-yl)benzamido)propyl)-2-(2-ami-nopyrimidin-5-yl)-7-methyl-4-morpholinothieno[3,2-d]pyrimidine-6-carboxamide;
(S)-1-(6-(4-((((2-(2-aminopyrimidin-5-yl)-4-morpholinothieno[3,2-d]pyrimidin-6-yl)methyl)(methyl)amino)methyl)phenyl)-2-methyl-3,4-dihydroquinolin-1(2H)-yl)ethan-1-one;
(S)-1-(6-(4-((((2-(2-aminopyrimidin-5-yl)-7-methyl-4-morpholinothieno[3,2-d]pyrimidin-6-yl)methyl)(methyl)amino)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)phenethyl)-6-(2-aminopyrimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)phenethyl)-2-(2-aminopyrimidin-5-yl)-4-morpholinothieno[3,2-d]pyrimidine-6-carboxamide;
(S)-N-(4-(1-acetyl-2-methyl-1,2,3,4-tetrahydroquinolin-6-yl)phenethyl)-2-(2-aminopyrimidin-5-yl)-7-methyl-4-morpholinothieno[3,2-d]pyrimidine-6-carboxamide;
(S)-N-(2-((4-(1-acetyl-2-methyl-1,2,3,4-tetrahydroquinolin-6-yl)phenyl)amino)-2-oxoethyl)-2-(2-aminopyrimidin-5-yl)-7-methyl-4-morpholinothieno[3,2-d]pyrimidine-6-carboxamide;
(S)-N-(2-((4-(1-acetyl-2-methyl-1,2,3,4-tetrahydroquinolin-6-yl)phenyl)amino)-2-oxoethyl)-6-(2-aminopyrimidin-5-yl)-3-methyl-8-morpholinoimidazo[1,2-a]pyrazine-2-carboxamide;
(S)-N-(2-((3-(1-acetyl-2-methyl-1,2,3,4-tetrahydroquinolin-6-yl)phenyl)amino)-2-oxoethyl)-2-(2-aminopyrimidin-5-yl)-7-methyl-4-morpholinothieno[3,2-d]pyrimidine-6-carboxamide;
(S)-N-(3-(1-acetyl-2-methyl-1,2,3,4-tetrahydroquinolin-6-yl)phenethyl)-6-(2-aminopyrimidin-5-yl)-3-methyl-8-morpholinoimidazo[1,2-a]pyrazine-2-carboxamide;
(S)-N-(3-(1-acetyl-2-methyl-1,2,3,4-tetrahydroquinolin-6-yl)phenethyl)-2-(2-aminopyrimidin-5-yl)-7-methyl-4-morpholinothieno[3,2-d]pyrimidine-6-carboxamide;
(S)-1-(6-(1-(1-((2-(2-aminopyrimidin-5-yl)-7-methyl-4-morpholinothieno[3,2-d]pyrimidin-6-yl)methyl)piperidin-4-yl)-1H-pyrazol-4-yl)-2-methyl-3,4-dihydroquinolin-1(2H)-yl)ethan-1-one;
(S)-1-(6-(1-(1-(2-(2-aminopyrimidin-5-yl)-7-methyl-4-morpholinothieno[3,2-d]pyrimidine-6-carbonyl)piperidin-4-yl)-1H-pyrazol-4-yl)-2-methyl-3,4-dihydroquinolin-1(2H)-yl)ethan-1-one;
tert-butyl (S)-(4-(1-acetyl-2-methyl-1,2,3,4-tetrahydroquinolin-6-yl)benzyl)((2-(2-aminopy-rimidin-5-yl)-7-methyl-4-morpholinothieno[3,2-d]pyrimidin-6-yl)methyl)carbamate;
(S)-1-(6-(4-((((2-(2-aminopyrimidin-5-yl)-7-methyl-4-morpholinothieno[3,2-d]pyrimidin-6-yl)methyl)amino)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)benzyl)-N-((2-(2-aminopyrim-idin-5-yl)-7-methyl-4-morpholinothieno[3,2-d]pyrimidin-6-yl)methyl)acetamide;
(S)-N-(4-(1-acetyl-2-methyl-1,2,3,4-tetrahydroquinolin-6-yl)benzyl)-6-(6-amino-4-(trifluoro-methyl)pyridin-3-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(6-methoxypyridin-3-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(1H-indazol-4-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(2-(difluoromethyl)-1H-benzo[d]imidazol-1-yl)-8-morpholinoimidazo[1,2-a]pyrazine-2-carboxamide;
(S)-N-(2-(4-(1-Acetyl-2-methyl-1,2,3,4-tetrahydroquinolin-6-yl)phenyl)propan-2-yl)-6-(2-aminopyrimidin-5-yl)-8-morpholinoimidazo[1,2-a]pyrazine-2-carboxamide;
N-((S)-1-(4-((S)-1-Acetyl-2-methyl-1,2,3,4-tetrahydroquinolin-6-yl)phenyl)ethyl)-6-(2-ami-nopyrimidin-5-yl)-8-morpholinoimidazo[1,2-a]pyrazine-2-carboxamide;
N-((R)-1-(4-((S)-1-Acetyl-2-methyl-1,2,3,4-tetrahydroquinolin-6-yl)phenyl)ethyl)-6-(2-ami-nopyrimidin-5-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-8-(piperidin-1-yl)imidazo[1,2-a]pyrazine-2-carboxamide;
(S)-N-(4-(1-Acetyl-2-methyl-1,2,3,4-tetrahydroquinolin-6-yl)benzyl)-2-(2-aminopyrimidin-5-yl)-4-morpholinopyrrolo[2,1-f][1,2,4]triazine-6-carboxamide;
(S)-N-(4-(1-Acetyl-2-methyl-1,2,3,4-tetrahydroquinolin-6-yl)benzyl)-2-(2-aminopyrimidin-5-yl)-7-methyl-4-morpholinopyrrolo[2,1-f][1,2,4]triazine-6-carboxamide;
(S)-N-(4-(1-Acetyl-2-methyl-1,2,3,4-tetrahydroquinolin-6-yl)benzyl)-6-(2-aminopyrimidin-5-yl)-4-morpholinopyrazolo[1,5-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-7-chloro-4-morpholinopyrazolo[1,5-a]pyrazine-2-carboxamide;
(S)-N-(4-(1-Acetyl-2-methyl-1,2,3,4-tetrahydroquinolin-6-yl)benzyl)-3-(2-aminopyrimidin-5-yl)-1-morpholinopyrrolo[1,2-a]pyrazine-7-carboxamide;
(S)-N-(4-(1-Acetyl-2-methyl-1,2,3,4-tetrahydroquinolin-6-yl)benzyl)-7-(2-aminopyrimidin-5-yl)-5-morpholinoimidazo[1,2-c]pyrimidine-2-carboxamide;
(S)-N-(4-(1-Acetyl-2-methyl-1,2,3,4-tetrahydroquinolin-6-yl)benzyl)-2-(2-aminopyrimidin-5-yl)-9-methyl-6-morpholino-9H-purine-8-carboxamide;
(S)-N-(4-(1-Acetyl-2-methyl-1,2,3,4-tetrahydroquinolin-6-yl)benzyl)-6-(2-aminopyrimidin-5-yl)-8-morpholino-[1,2,4]triazolo[1,5-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-8-morpholino-[1,2,4]triazolo[1,5-a]pyridine-2-carboxamide;
(S)-N-(4-(1-Acetyl-2-methyl-1,2,3,4-tetrahydroquinolin-6-yl)benzyl)-6-(2-aminopyrimidin-5-yl)-8-morpholinoimidazo[1,2-a]pyridine-2-carboxamide;
(S)-N-(4-(1-Acetyl-2-methyl-1,2,3,4-tetrahydroquinolin-6-yl)benzyl)-5-(2-aminopyrimidin-5-yl)-7-morpholinopyrazolo[1,5-a]pyrimidine-2-carboxamide;
N-(4-(1-Acetyl-2-methylindolin-5-yl)benzyl)-6-(2-aminopyrimidin-5-yl)-8-morpholinoimidazo[1,2-a]pyrazine-2-carboxamide;
N-(4-(1-Acetyl-2-methyl-4-propoxyindolin-5-yl)benzyl)-6-(2-aminopyrimidin-5-yl)-8-morpholinoimidazo[1,2-a]pyrazine-2-carboxamide; and
(S)-1-(6-(4-((((6-(2-Aminopyrimidin-5-yl)-8-morpholinoimidazo[1,2-a]pyrazin-2-yl)methyl)(hydroxy)amino)methyl)phenyl)-2-methyl-3,4-dihydroquinolin-1(2H)-yl)ethan-1-one.

### Therapeutic Methods and Uses

The compounds of Formula (I), or a pharmaceutically acceptable salt, solvate or stereoisomer thereof, of the present disclosure, and pharmaceutical compositions comprising the compounds of Formula (I) or a pharmaceutically acceptable salt, solvate or stereoisomer thereof, find use in the therapy of diseases, for example, Myc-dependent disorders (e.g., cancers). Accordingly, there is also provided a compound of Formula (I), or a pharmaceutically acceptable salt, solvate or stereoisomer thereof as described herein, or pharmaceutical composition as described herein, for use in therapy. In one example, a compound of Formula (I) or a pharmaceutically acceptable salt, solvate or stereoisomer thereof, or a pharmaceutical composition as described herein, finds use in the prevention or treatment of cancer.

A compound of Formula (I) or a pharmaceutically acceptable salt, solvate or stereoisomer thereof, or a pharmaceutical composition as described herein, finds use in the treatment of diseases for which inhibition of a kinase and/or a bromodomain-containing protein, provides a therapeutic effect. In one example, the kinase includes, but is not limited to, PI3K. In one ex ample, the PI3K includes, but is not limited to, PI3Kα, PI3Kβ, PI3Kγ and PI3Kδ. In one exam ple, the bromodomain-containing protein includes, but is not limited to, a BET protein. In one example, the BET protein includes, but is not limited to, BRD2, BRD3, BRD4 and BRDT.

In one example, the compounds of Formula (I) or a pharmaceutically acceptable salt, solvate or stereoisomer thereof, or a pharmaceutical composition as described herein, finds use in the treatment of diseases for which inhibition of PI3K and a bromodomain-containing protein, provides a therapeutic effect. In one example, the compounds of Formula (I) or a pharmaceutically acceptable salt, solvate or stereoisomer thereof, or a pharmaceutical composition as described herein, finds use in the treatment of diseases for which inhibition of PI3K and a BET protein, provides a therapeutic effect. In one example, the compounds of Formula (I) or a pharmaceutically acceptable salt, solvate or stereoisomer thereof, or a pharmaceutical composition as described herein, finds use in the treatment of diseases for which inhibition of PI3K and a BRD4 protein, provides a therapeutic effect. In one example, the compounds of Formula (I) or a pharmaceutically acceptable salt, solvate or stereoisomer thereof, or a pharmaceutical composition as described herein, finds use in the treatment of diseases for which inhibition of PI3Kα, PI3Kβ, PI3Kγ and/or PI3Kδ, and a BRD4 protein, provides a therapeutic effect. In one example, diseases for which inhibition of a kinase and/or a bromodomain-containing protein provides a therapeutic effect are Myc-dependent disorders.

In one example, a compound of Formula (I) or a pharmaceutically acceptable salt, solvate or stereoisomer thereof, or a pharmaceutical composition as described herein, finds use in the treatment of Myc-dependent disorders.

### Myc-Dependent Disorders

Accordingly, there is provided a method of preventing or treating a Myc-dependent dis order in a subject, comprising administering an effective amount of the compound of Formula (I) or a pharmaceutically acceptable salt, solvate or stereoisomer thereof, or a pharmaceutical composition as described herein, to the subject.

Examples of Myc-dependent disorders include, but are not limited to, proliferative disorders (e.g., cancers), autoimmune diseases, and viral infections. In one example, the Myc-dependent disorder is a Myc-dependent proliferative disease. In one example, the Myc-dependent disorder is cancer, such as a Myc-dependent cancer.

Accordingly, there is provided a method of preventing or treating a Myc-dependent cancer in a subject, comprising administering an effective amount of the compound of Formula (I) or a pharmaceutically acceptable salt, solvate or stereoisomer thereof, or a pharmaceutical composition as described herein, to the subject.

In one example, the Myc-dependent cancer is a haematological cancer. In one example, the Myc-dependent cancer is a non-haematological cancer.

Examples of Myc-dependent cancers include, but are not limited to, adrenal cancer, acinic cell carcinoma, acoustic neuroma, acral lentiginous melanoma, acrospiroma, acute erythroid leukaemia, acute lymphoblastic leukaemia, acute myeloblastic leukaemia, acute megakaryoblastic leukaemia, acute monoblastic and monocytic leukaemia, acute promyelocy-tic leukaemia, adenocarcinoma, adenoid cystic carcinoma, adenoma, adenomatoid odontogenic tumor, adenosquamous carcinoma, adipose tissue neoplasm, adrenocortical carcinoma, adult T-cell leukaemia/lymphoma, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukaemia, B-cell prolymphocytic leukaemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chon-droma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endo-dermal sinus tumor, enteropathy-associated T-cell lymphoma, oesophageal cancer, fetus in fetu, fibroma, fibrosarcoma, follicular lymphoma, follicular thyroid cancer, ganglioneuroma, gastro intestinal cancer, germ cell tumor, gestational choriocarcinoma, giant cell fibroblastoma, giant cell tumor of the bone, glial tumor, glioblastoma multiforme, glioma, gliomatosis cerebri, glu-cagonoma, gonadoblastoma, granulosa cell tumor, gynandroblastoma, gallbladder cancer, gas-tric cancer, hairy cell leukaemia, haemangioblastoma, head and neck cancer, haemangiopericy-toma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangio sarcoma, lymphoepithelioma, lymphoma, chronic lymphocytic leukaemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukaemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, na-sopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peri-toneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Ser-toli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, ver-rucous carcinoma, visual pathway glioma, vulvar cancer, vaginal cancer, Waldenstrom's mac-roglobulinemia, Warthin's tumor, and Wilms' tumour.

In one example, the Myc-dependent cancer is selected from the group consisting of lymphoma, acute myeloid leukaemia, multiple myeloma, neuroblastoma, and medulloblastoma. In one example, the Myc-dependent cancer is multiple myeloma.

Accordingly, there is provided a method of preventing or treating a cancer in a subject, comprising administering an effective amount of the compound of Formula (I) or a pharmaceutically acceptable salt, solvate or stereoisomer thereof, or a pharmaceutical composition as described herein, to the subject, wherein the cancer is selected from the group consisting of lymphoma, acute myeloid leukaemia, multiple myeloma, neuroblastoma, and medulloblastoma.

Accordingly, there is provided a method of preventing or treating a Myc-dependent cancer in a subject, comprising administering an effective amount of the compound of Formula (I) or a pharmaceutically acceptable salt, solvate or stereoisomer thereof, or a pharmaceutical composition as described herein, to the subject, wherein the Myc-dependent cancer is selected from the group consisting of lymphoma, acute myeloid leukaemia, multiple myeloma, neuroblastoma, and medulloblastoma.

In one example, there is provided a compound of Formula (I) or a pharmaceutically acceptable salt, solvate or stereoisomer thereof, or a pharmaceutical composition as described herein, for use in the prevention and/or treatment of multiple myeloma.

A compound of Formula (I) may be provided as an agent, such as an anticancer agent or a dual protein kinase and bromodomain-containing protein inhibitor agent.

In one example, the compound of Formula (I), as described herein, is used in therapy (e.g., in the therapy of Myc-dependent disorders).

### Compositions

Whilst a compound of Formula (I), or salt, solvate or stereoisomer thereof, may, in some embodiments, be administered alone, it is more typically administered as part of a pharmaceutical composition or formulation. Thus, the present disclosure also provides a pharmaceutical composition comprising a compound of Formula (I), or a salt, solvate or stereoisomer, and a pharmaceutically acceptable excipient. The pharmaceutical composition comprises one or more pharmaceutically acceptable diluents, carriers or excipients (collectively referred to herein as “excipient” materials).

The present disclosure also provides pharmaceutical formulations or compositions, both for veterinary and for human medical use, which comprise compounds of Formula (I), or a salt, solvate or stereoisomer thereof, of the present disclosure, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof.

Examples of pharmaceutical formulations include those suitable for oral, parenteral (including subcutaneous, intradermal, intramuscular, intravenous, and intraarticular), inhalation (including fine particle dusts or mists that may be generated by means of various types of me-tered dose pressurised aerosols), nebulisers or insufflators, rectal, intraperitoneal and topical (including dermal, buccal, sublingual, and intraocular) administration, although the most suitable route may depend upon, for example, the condition and disorder of the recipient.

The pharmaceutical formulations may conveniently be presented in unit dosage form and may be prepared by any of the methods well known in the art of pharmacy. All methods include the step of brining a compound of Formula (I), or salt, solvate or stereoisomer thereof, into association with the excipient that constitutes one or more necessary ingredients. In general, the formulations are prepared by uniformly and intimately bringing into association the active ingredient with liquid carriers or finely divided solid carriers or both, and then, if necessary, shaping the product into the desired formulation.

In some embodiments, that composition is formulated for oral delivery. For example, pharmaceutical formulations of the present disclosure suitable for oral administration may be presented as discrete units such as capsules, cachets, pills or tablets each containing a predeter-mined amount of the active ingredient; as a powder or granules, as a solution or a suspension in an aqueous liquid or non-aqueous liquid, for example as elixirs, tinctures, suspensions or syrups; or as an oil-in-water liquid emulsion or a water-in-oil liquid emulsion. A compound of Formula (I), or salt, solvate or stereoisomer thereof, may also be presented as a bolus, electuary or paste.

A tablet may be made, for example, by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Moulded tablets may be made by moulding in a suitable machine a mixture of the powdered compound moistened with an inert liquid diluent. The tablets may be optionally coated or scored, and may be formulated so as to provide slow or controlled release of the compound of Formula (I), or salt, solvate or stereoisomer thereof. The compound of Formula (I), or salt, solvate or stereoisomer thereof, can, for example, be administered in a form suitable for immediate release or extended release. Immediate release or extended release can be achieved by the use of suitable pharmaceutical compositions comprising a compound of Formula (I), or salt, solvate or stereoisomer thereof, or, particularly in the case of extended release, by the use of devices such as subcutaneous implants or osmotic pumps. A compound of Formula (I), or salt, solvate or stereoisomer thereof, may also be administered liposomally.

For example, in one embodiment, the formulation may be a sterile, lyophilized composition that is suitable for reconstitution in an aqueous vehicle prior to injection. In one embodiment, a formulation suitable for parenteral administration conveniently comprises a sterile aqueous preparation of the compound of Formula (I), or salt, solvate or stereoisomer thereof, which may for example be formulated to be isotonic with the blood of the recipient.

The compounds of Formula (I), or salt, solvate or stereoisomer thereof, of the present disclosure may for example be formulated in compositions including those suitable for inhalation to the lung, by aerosol, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the compound of Formula (I), or salt, solvate or stereoisomer thereof, into association with a carrier that constitutes one or more accessory ingredients. In general, the compositions are prepared by bringing the compound of Formula (I), or salt, solvate or stereoisomer thereof, into association with a liquid carrier to form a solution or a suspension, or alternatively, bring the compound of Formula (I), or salt, solvate or stereoisomer thereof, into association with formulation components suitable for forming a solid, optionally a particulate product, and then, if warranted, shaping the product into a desired delivery form. Solid formulations of the present disclosure, when particulate, will typically comprise particles with sizes ranging from about 1 nanometer to about 500 microns. In general, for solid formulations intended for intravenous administration, particles will typically range from about 1 nm to about 10 microns in diameter. The composition may contain compounds of Formula (I) of the present disclosure that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In one example, the composition contains compounds of Formula (I) with a mean size of between 5 and 20 nm. In one example, the compound of Formula (I) is polydispersed in the composition, with PDI of between 1.01 and 1.8, especially between 1.01 and 1.5, and more especially between 1.01 and 1.2. In one example, the compounds of Formula (I) are monodispersed in the composition.

It should be understood that in addition to the ingredients particularly mentioned above, the formulations may include other agents conventional in the art having regard to the type of formulation in question, for example, those suitable for oral administration may include fla-vouring agents.

The compositions of the present disclosure may also include polymeric excipients/ad-ditives or carriers, e.g., polyvinylpyrrolidones, derivatised celluloses such as hydroxymethyl-cellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, citrate, trehalose, binders, disintegrants, thick-eners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidyl-ethanolamines, fatty acids and fatty esters, steroids (e.g., cholesterol)), and chelating agents (e.g., EDTA, zinc and other such suitable cations). Other pharmaceutical excipients and/or ad ditives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

### Dosages

The amount of active ingredient that is required to achieve a therapeutic effect will, of course, vary with the particular compound, the route of administration, the subject under treatment, including the type, species, age, weight, sex, and medical condition of the subject being treated, and the renal and hepatic function of the subject, and the particular condition, disorder or disease being treated, as well as its severity. An ordinary skilled physician or clinician can readily determine and prescribe the effective amount of the drug required to prevent or treat the condition, disorder or disease.

Dosages of a compound of Formula (I), or salt, solvate or stereoisomer thereof, when used for the indicated effects, will range between, for example, about 0.01 mg per kg of body weight per day (mg/kg/day) to about 1000 mg/kg/day. In one example, the dosage of a compound of Formula (I), or salt, solvate or stereoisomer thereof, is between about 0.01 and 1000, 0.1 and 500, 0.1 and 100, 1 and 50 mg/kg/day. In one example, the dosage of a compound of Formula (I), or salt, solvate or stereoisomer thereof, is between about 0.01 and 1000 mg/kg/day. In one example, the dosage of a compound of Formula (I), or salt, solvate or stereoisomer thereof, is between about 0.1 and 100 mg/kg/day. In one example, the dosage of a compound of Formula (I), or salt, solvate or stereoisomer thereof, is greater than about 0.01, 0.1, 1, 10, 20, 50, 75, 100, 500, 1000 mg/kg/day. In one example, the dosage of a compound of Formula (I), or salt, solvate or stereoisomer thereof, is greater than about 0.01 mg/kg/day. In one example, the dosage of a compound of Formula (I), or salt, solvate or stereoisomer thereof, is less than about 5000, 1000, 75, 50, 20, 10, 1, 0.1 mg/kg/day. In one example, the dosage of a compound of Formula (I), or salt, solvate or stereoisomer thereof, is less than about 1000 mg/kg/day.

A compound of Formula (I), or salt, solvate or stereoisomer thereof, may for example be administered as a single daily dose, or otherwise the total daily dosage may be administered in divided doses of two, three, or four times daily. In one example, the compound of Formula (I), or salt, solvate or stereoisomer thereof, may be dosed less frequently than once per day, for example once per two days, three days, four days, five days, six days, or once per week.

If administered intravenously, an infusion of the compound over a period of time may be used, for example. Furthermore, a compound of Formula (I), or salt, solvate or stereoisomer thereof, may be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

### Combination Therapy

Whilst a compound of Formula (I), or salt, solvate or stereoisomer thereof, may be used as the sole active agent in a medicament, it is also possible for a compound of Formula (I), or salt thereof, to be used in combination with one or more further therapeutic agents. Accordingly, in one example, a compound of Formula (I), or salt, solvate or stereoisomer thereof, is used in combination with one or more further therapeutic agents. The present disclosure therefore also provides a combination of a compound of Formula (I), or salt, solvate or stereoisomer thereof, and a further therapeutic agent. The present disclosure also provides a pharmaceutical composition comprising a combination of a compound of Formula (I), or salt, solvate or stereoisomer thereof, a further therapeutic agent, and a pharmaceutically acceptable excipient. Such one or more further therapeutic agents may for example be anti-cancer agents. Drugs are often co-administered with other drugs during chemotherapy. In one example, a compound of Formula (I), or salt, solvate or stereoisomer thereof, is used in combination with one or more further anti cancer agents. In one example, a compound of Formula (I), or salt, solvate or stereoisomer thereof, is used in combination with one or more further cytotoxic agents.

Examples of cytotoxic agents include, but are not limited to, alkylating agents (including, without limitation, nitrogen mustards, ethyl enimine derivatives, alkyl sulfonates, nitro-soureas and triazenes): uracil mustard, chlormethine, cyclophosphamide (Cytoxan®), ifosfamide, melphalan, chlorambucil, pipobroman, triethylene-melamine, triethylenethiophos-phoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors), methotrexate, 5-fluorouracil, floxuridine, cytara-bine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, bleomycin, dactinomycin, daunorubicin, doxorubicin, epirubicin, idarubicin, ara-c, paclitaxel (paclitaxel is commercially available as Taxol®), mithramycin, deoxyco-formycin, mitomycin-c, 1-aspara-ginase, interferons (preferably IFN-. alpha.), etoposide, and teniposide. Other proliferative cytotoxic agents are navelbene, CPT-11, anastrazole, letrazole, capecitabine, reloxafine, yclo-phosphamide, ifosamide, and droloxafine.

The compound of Formula (I), or salt, solvate or stereoisomer thereof, and the one or more further pharmaceutically active agents may be administered simultaneously, subsequently or separately. For example, they may be administered as part of the same composition, or by administration of separate compositions. The one or more further pharmaceutically active agents may for example be anti-cancer agents for therapy of chronic lymphocytic leukaemia (CLL), acute myeloid leukaemia, multiple myeloma, neuroblastoma, lymphoma, Hodgkin's lymphoma, non-Hodgkin's lymphoma, large B-cell lymphoma, and/or medulloblastoma.

In one example, the compound of Formula (I), or salt, solvate or stereoisomer, is administered in combination with one or more further anticancer agents. In one example, the compound of Formula (I), or salt, solvate or stereoisomer, is administered in combination with one or more further cytotoxic agents.

The further therapeutic agents, when employed in combination with a compound of Formula (I), or salt thereof, may be used for example in those amounts indicated in the Physi-cians' Desk Reference or as otherwise determined by one of ordinary skill in the art.

### Synthesis of Compounds of Formula (I)

Numerous synthetic routes to the compounds of Formula (I) can be devised by any person skilled in the art and the possible synthetic routes described below are not intended to be limiting. Possible synthetic routes for the compounds of Formula (I) are shown schematically below. Where appropriate, any initially produced compound of Formula (I) can be converted into another compound of Formula (I) by known methods.

A compound of Formula (I) of the present disclosure may for example be prepared by any suitable method, for example by a) reacting a compound of Formula (IV) with a compound of Formula (V):

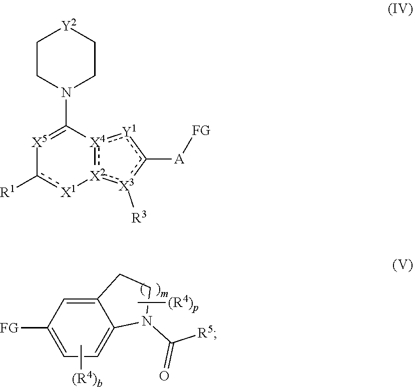

wherein X¹, X², X³, X⁴, X⁵, Y¹, Y², R¹, R², R³, R⁴, R⁵, m, n, p, b, A, R⁶, and R⁷ are as described herein; and wherein FG are complimentary, reactive functional groups.

A compound of Formula (I) of the present disclosure may for example be prepared by any suitable method, for example by a) reacting a compound of Formula (IV) with a compound of Formula (V):

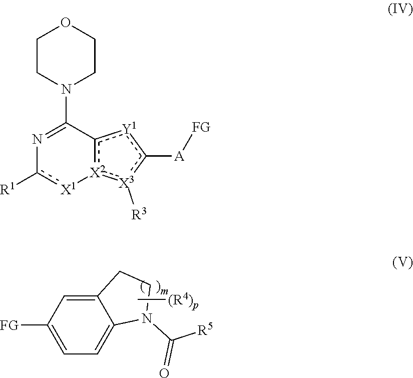

wherein X¹, X², X³, Y¹, R¹, R², R³, R⁴, R⁵, m, n, p, A, R⁶, and R⁷ are as described herein; and
wherein FG are complimentary, reactive functional groups.

A compound of Formula (I) of the present disclosure may also for example be prepared by any suitable method, for example by a) reacting a compound of Formula (VI) with a compound of Formula (VII):

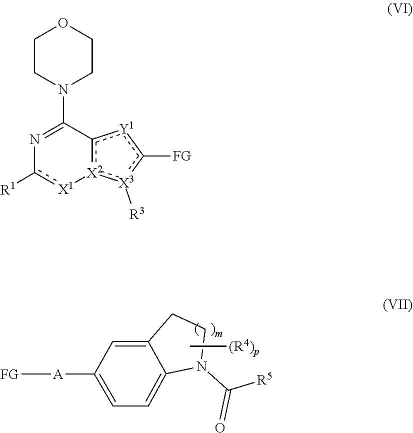

wherein X¹, X², X³, Y¹, R¹, R², R³, R⁴, R⁵, m, n, p, A, R⁶, and R⁷ are as described herein; and
wherein FG are complimentary, reactive functional groups.

The present disclosure will now be described with reference to the following examples that illustrate some particular aspects of the present disclosure. However, it is to be understood that the particularity of the following description of the present disclosure is not to supersede the generality of the preceding description of the present disclosure.

### EXAMPLES

The following Table provides compound numbers and nomenclature, with reference to its molecular structure, and is adhered to herein:

| | | |
|---|---|---|
| [Cpd 001] | 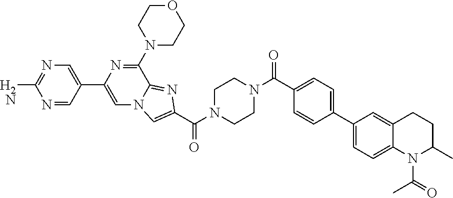 [SMILES: CC(=O)N1c2ccc(-c3ccc(C(=O)N4CCN(C(=O)c5cn6cc(-c7cnc(N)nc7)nc(N7CCOCC7)c6n5)CC4)cc3)cc2CCC1C] | 1-(6-(4-(4-(6-(2-Aminopy- rimidin-5-yl)-8-morpho- linoimidazo[1,2-a]pyrazine- 2-carbonyl)piperazine-1- carbonyl)phenyl)-2-methyl- 3,4-dihydroquinolin-1(2H)- yl)ethan-1-one |
| [Cpd 002] | 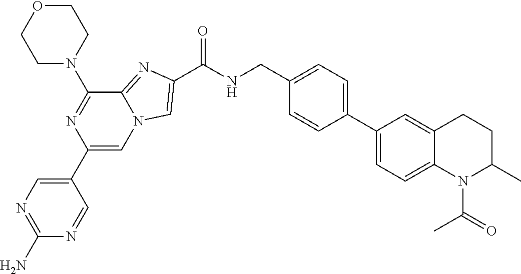 [SMILES: CC(=O)N1c2ccc(-c3ccc(CNC(=O)c4cn5cc(-c6cnc(N)nc6)nc(N6CCOCC6)c5n4)cc3)cc2CCC1C] | N-(4-(1-Acetyl-2-methyl- 1,2,3,4-tetrahydroquinolin- 6-yl)benzyl)-6-(2-aminopy- rimidin-5-yl)-8-morpho- linoimidazo[1,2-a]pyrazine- 2-carboxamide |
| [Cpd 003] | 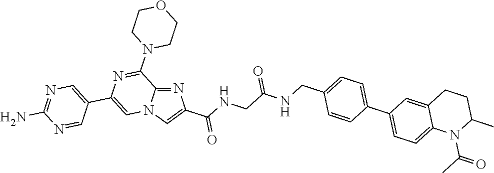 [SMILES: CC(=O)N1c2ccc(-c3ccc(CNC(=O)CNC(=O)c4cn5cc(-c6cnc(N)nc6)nc(N6CCOCC6)c5n4)cc3)cc2CCC1C] | N-(2-((4-(1-Acetyl-2-me- thyl-1,2,3,4-tetrahydroquin- olin-6-yl)benzyl)amino)-2- oxoethyl)-6-(2-aminopyrim- idin-5-yl)-8-morpholinoim- idazo[1,2-a]pyrazine-2-car- boxamide |
| [Cpd 004] | 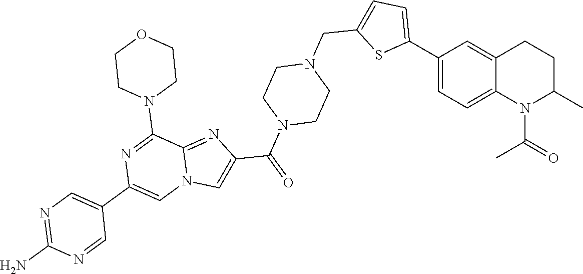 [SMILES: CC(=O)N1c2ccc(-c3ccc(CN4CCN(C(=O)c5cn6cc(-c7cnc(N)nc7)nc(N7CCOCC7)c6n5)CC4)s3)cc2CCC1C] | 1-(6-(5-((4-(6-(2-Aminopy- rimidin-5-yl)-8-morpho- linoimidazo[1,2-a]pyrazine- 2-carbonyl)piperazin-1- yl)methyl)thiophen-2-yl)-2- methyl-3,4-dihydroquinolin- 1(2H)-yl)ethan-1-one |
| [Cpd 005] | 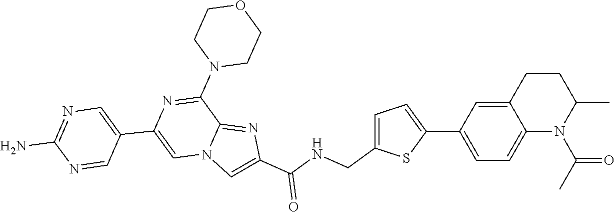 [SMILES: CC(=O)N1c2ccc(-c3ccc(CNC(=O)c4cn5cc(-c6cnc(N)nc6)nc(N6CCOCC6)c5n4)s3)cc2CCC1C] | N-((5-(1-Acetyl-2-methyl- 1,2,3,4-tetrahydroquinolin- 6-yl)thiophen-2-yl)methyl)- 6-(2-aminopyrimidin-5-yl)- 8-morpholinoimidazo[1,2- a]pyrazine-2-carboxamide |
| [Cpd 006] | 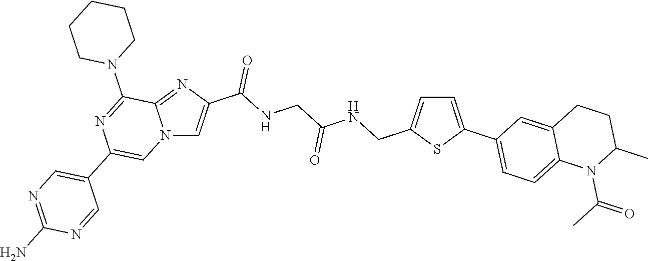 [SMILES: CC(=O)N1c2ccc(-c3ccc(CNC(=O)CNC(=O)c4cn5cc(-c6cnc(N)nc6)nc(N6CCCCC6)c5n4)s3)cc2CCC1C] | N-(2-(((5-(1-Acetyl-2-me- thyl-1,2,3,4-tetrahydroquin- olin-6-yl)thiophen-2-yl)me- thyl)amino)-2-oxoethyl)-6- (2-aminopyrimidin-5-yl)-8- morpholinoimidazo[1,2- a]pyrazine-2-carboxamide |
| [Cpd 007] | 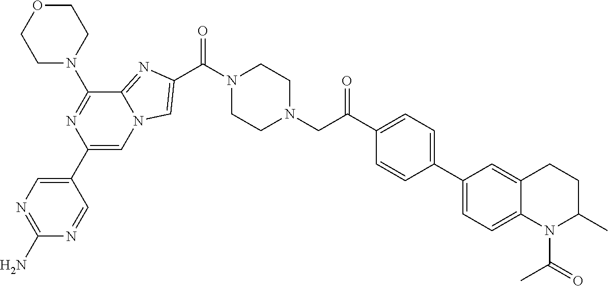 [SMILES: CC(=O)N1c2ccc(-c3ccc(C(=O)CN4CCN(C(=O)c5cn6cc(-c7cnc(N)nc7)nc(N7CCOCC7)c6n5)CC4)cc3)cc2CCC1C] | 1-(4-(1-Acetyl-2-methyl- 1,2,3,4-tetrahydroquinolin- 6-yl)phenyl)-2-(4-(6-(2-ami- nopyrimidin-5-yl)-8-mor- pholinoimidazo[1,2-a]pyra- zine-2-carbonyl)piperazin- 1-yl)ethan-1-one |
| [Cpd 008] | 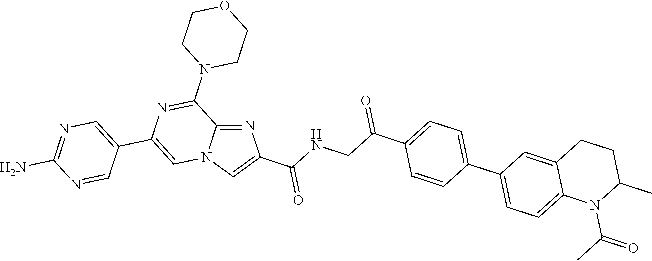 [SMILES: CC(=O)N1c2ccc(-c3ccc(C(=O)CNC(=O)c4cn5cc(-c6cnc(N)nc6)nc(N6CCOCC6)c5n4)cc3)cc2CCC1C] | N-(2-(4-(1-Acetyl-2-methyl- 1,2,3,4-tetrahydroquinolin- 6-yl)phenyl)-2-oxoethyl)-6- (2-aminopyrimidin-5-yl)-8- morpholinoimidazo[1,2- a]pyrazine-2-carboxamide |
| [Cpd 009] | 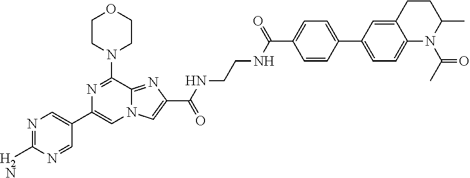 [SMILES: CC(=O)N1c2ccc(-c3ccc(C(=O)NCCNC(=O)c4cn5cc(-c6cnc(N)nc6)nc(N6CCOCC6)c5n4)cc3)cc2CCC1C] | N-(2-(4-(1-Acetyl-2-methyl- 1,2,3,4-tetrahydroquinolin- 6-yl)benzamido)ethyl)-6-(2- aminopyrimidin-5-yl)-8- morpholinoimidazo[1,2- a]pyrazine-2-carboxamide |
| [Cpd 010] | 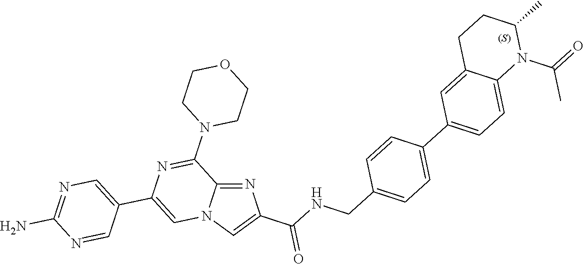 | (S)-N-(4-(1-Acetyl-2-me- thyl-1,2,3,4-tetrahydroquin- olin-6-yl)benzyl)-6-(2-ami- nopyrimidin-5-yl)-8-mor- pholinoimidazo[1,2-a]pyra- zine-2-carboxamide |
| [Cpd 011] | 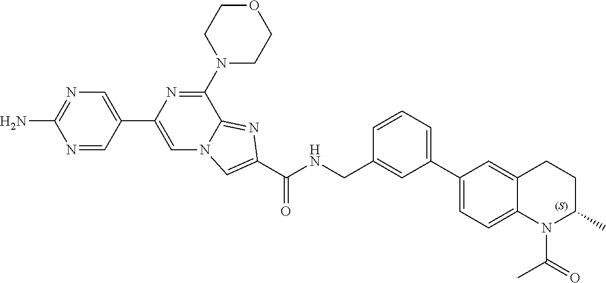 | (S)-N-(3-(1-Acetyl-2-me- thyl-1,2,3,4-tetrahydroquin- olin-6-yl)benzyl)-6-(2-ami- nopyrimidin-5-yl)-8-mor- pholinoimidazo[1,2-a]pyra- zine-2-carboxamide |
| [Cpd 012] | 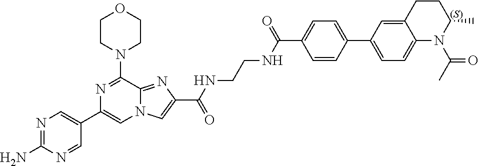 | (S)-N-(2-(4-(1-Acetyl-2-me- thyl-1,2,3,4-tetrahydroquin- olin-6-yl)benzamido)ethyl)- 6-(2-aminopyrimidin-5-yl)- 8-morpholinoimidazo[1,2- a]pyrazine-2-carboxamide |
| [Cpd 013] | 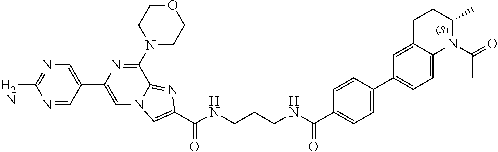 | (S)-N-(3-(4-(1-Acetyl-2-me- thyl-1,2,3,4-tetrahydroquin- olin-6-yl)benzamido)pro- pyl)-6-(2-aminopyrimidin- 5-yl)-8-morpholinoimid- azo[1,2-a]pyrazine-2-car- boxamide |
| [Cpd 014] | 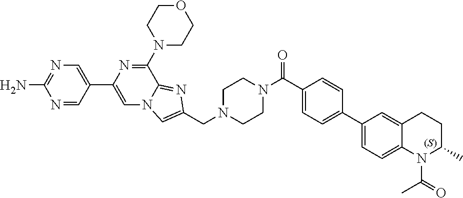 | (S)-1-(6-(4-(4-((6-(2-Ami- nopyrimidin-5-yl)-8-mor- pholinoimidazo[1,2-a]pyra- zin-2-yl)methyl)piperazine- 1-carbonyl)phenyl)-2-me- thyl-3,4-dihydroquinolin- 1(2H)-yl)ethan-1-one |
| [Cpd 015] | 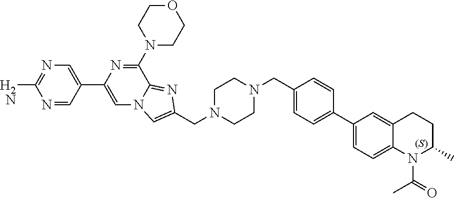 | (S)-1-(6-(4-((4-((6-(2-Ami- nopyrimidin-5-yl)-8-mor- pholinoimidazo[1,2-a]pyra- zin-2-yl)methyl)piperazin-1- yl)methyl)phenyl)-2-me- thyl-3,4-dihydroquinolin- 1(2H)-yl)ethan-1-one |
| [Cpd 016] | 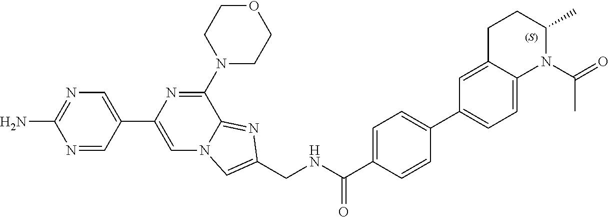 | (S)-4-(1-Acetyl-2-methyl- 1,2,3,4-tetrahydroquinolin- 6-yl)-N-((6-(2-aminopyrim- idin-5-yl)-8-morpholinoim- idazo[1,2-a]pyrazin-2- yl)methyl)benzamide |
| [Cpd 017] | 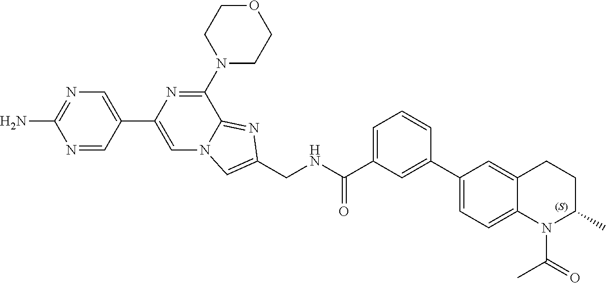 | (S)-3-(1-Acetyl-2-methyl- 1,2,3,4-tetrahydroquinolin- 6-yl)-N-((6-(2-aminopyrim- idin-5-yl)-8-morpholinoim- idazo[1,2-a]pyrazin-2- yl)methyl)benzamide |
| [Cpd 018] | 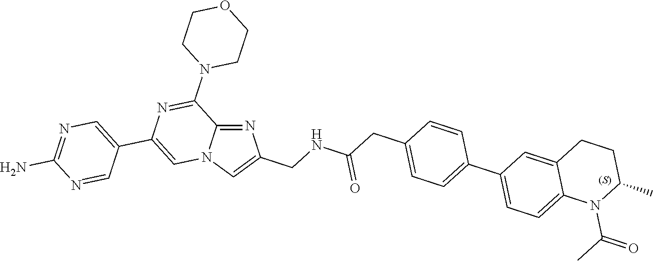 | (S)-2-(4-(1-Acetyl-2-me- thyl-1,2,3,4-tetrahydroquin- olin-6-yl)phenyl)-N-((6-(2- aminopyrimidin-5-yl)-8- morpholinoimidazo[1,2- a]pyrazin-2-yl)methyl)acet- amide |
| [Cpd 019] | 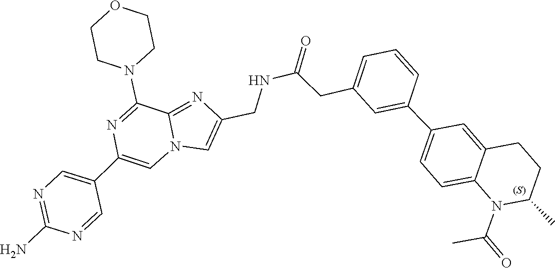 | (S)-2-(3-(1-Acetyl-2-me- thyl-1,2,3,4-tetrahydroquin- olin-6-yl)phenyl)-N-((6-(2- aminopyrimidin-5-yl)-8- morpholinoimidazo[1,2- a]pyrazin-2-yl)methyl)acet- amide |
| [Cpd 020] | 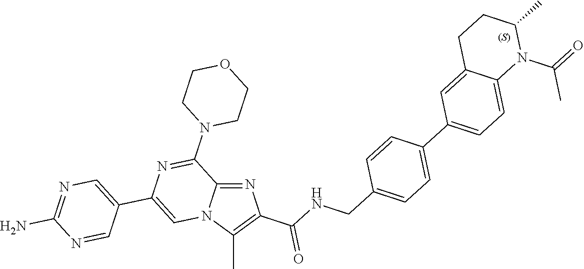 | (S)-N-(4-(1-Acetyl-2-me- thyl-1,2,3,4-tetrahydroquin- olin-6-yl)benzyl)-6-(2-ami- nopyrimidin-5-yl)-3-methyl- 8-morpholinoimidazo[1,2- a]pyrazine-2-carboxamide |
| [Cpd 021] | 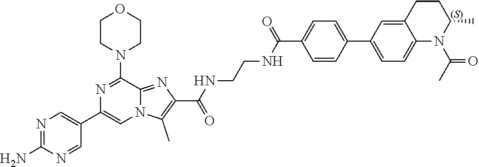 | (S)-N-(2-(4-(1-Acetyl-2-me- thyl-1,2,3,4-tetrahydroquin- olin-6-yl)benzamido)ethyl)- 6-(2-aminopyrimidin-5-yl)- 3-methyl-8-morpholinoim- idazo[1,2-a]pyrazine-2-car- boxamide |
| [Cpd 022] | 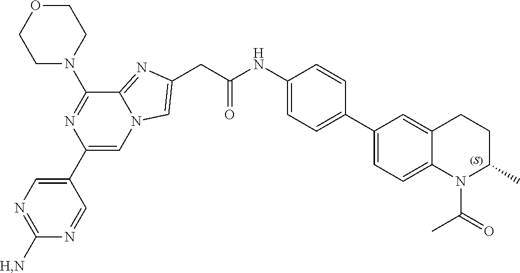 | (S)-N-(4-(1-Acetyl-2-me- thyl-1,2,3,4-tetrahydroquin- olin-6-yl)phenyl)-2-(6-(2- aminopyrimidin-5-yl)-8- morpholinoimidazo[1,2- a]pyrazin-2-yl)acetamide |
| [Cpd 023] | 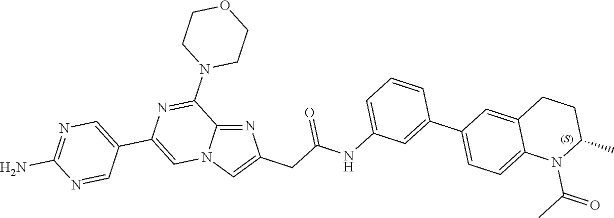 | (S)-N-(3-(1-Acetyl-2-me- thyl-1,2,3,4-tetrahydroquin- olin-6-yl)phenyl)-2-(6-(2- aminopyrimidin-5-yl)-8- morpholinoimidazo[1,2- a]pyrazin-2-yl)acetamide |
| [Cpd 024] | 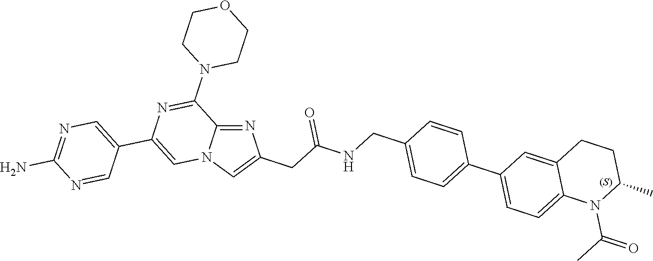 | (S)-N-(4-(1-Acetyl-2-me- thyl-1,2,3,4-tetrahydroquin- olin-6-yl)benzyl)-2-(6-(2- aminopyrimidin-5-yl)-8- morpholinoimidazo[1,2- a]pyrazin-2-yl)acetamide |
| [Cpd 025] | 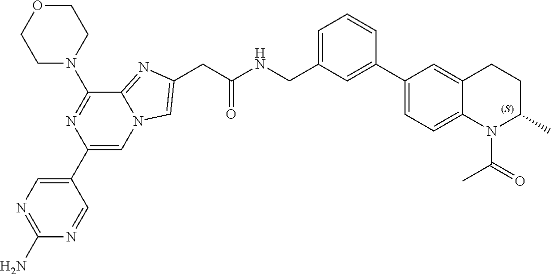 | (S)-N-(3-(1-Acetyl-2-me- thyl-1,2,3,4-tetrahydroquin- olin-6-yl)benzyl)-2-(6-(2- aminopyrimidin-5-yl)-8- morpholinoimidazo[1,2- a]pyrazin-2-yl)acetamide |
| [Cpd 026] | 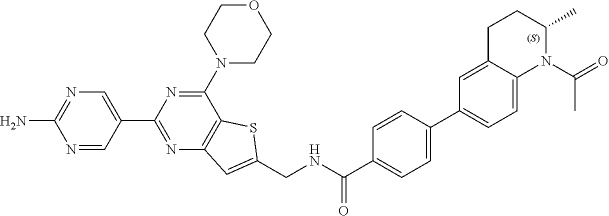 | (S)-4-(1-Acetyl-2-methyl- 1,2,3,4-tetrahydroquinolin- 6-yl)-N-((2-(2-aminopyrim- idin-5-yl)-4-morpho- linothieno[3,2-d]pyrimidin- 6-yl)methyl)benzamide |
| [Cpd 027] | 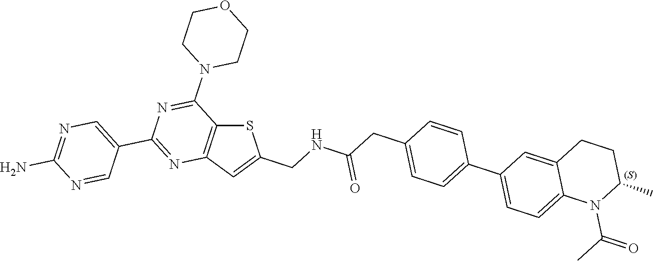 | (S)-2-(4-(1-Acetyl-2-me- thyl-1,2,3,4-tetrahydroquin- olin-6-yl)phenyl)-N-((2-(2- aminopyrimidin-5-yl)-4- morpholinothieno[3,2-d]py- rimidin-6-yl)methyl)acetam- ide |
| [Cpd 028] | 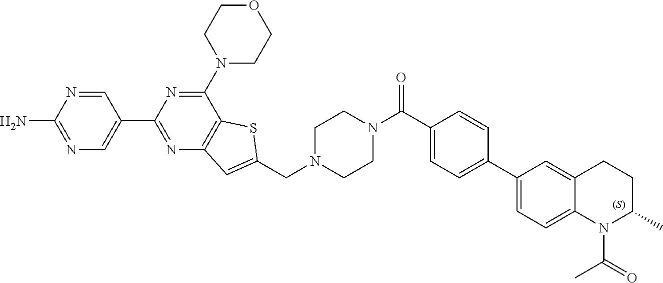 | (S)-1-(6-(4-(4-((2-(2-Ami- nopyrimidin-5-yl)-4-mor- pholinothieno[3,2-d]pyrim- idin-6-yl)methyl)piperazine- 1-carbonyl)phenyl)-2-me- thyl-3,4-dihydroquinolin- 1(2H)-yl)ethan-1-one |
| [Cpd 029] | 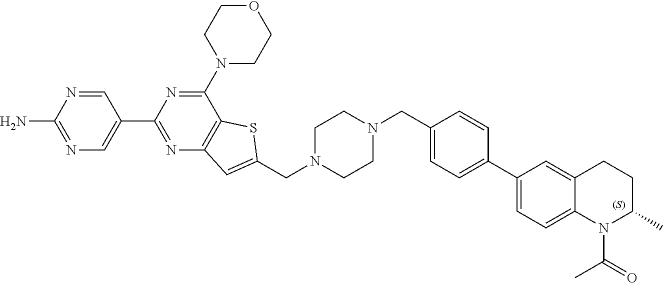 | (S)-1-(6-(4-((4-((2-(2-Ami- nopyrimidin-5-yl)-4-mor- pholinothieno[3,2-d]pyrim- idin-6-yl)methyl)piperazin- 1-yl)methyl)phenyl)-2-me- thyl-3,4-dihydroquinolin- 1(2H)-yl)ethan-1-one |
| [Cpd 030] | 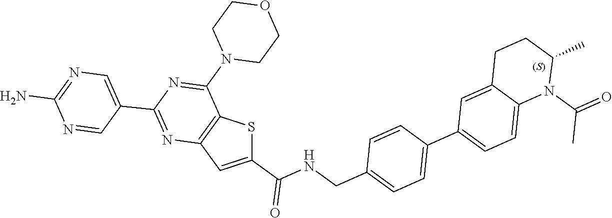 | (S)-N-(4-(1-Acetyl-2-me- thyl-1,2,3,4-tetrahydroquin- olin-6-yl)benzyl)-2-(2-ami- nopyrimidin-5-yl)-4-mor- pholinothieno[3,2-d]pyrimi- dine-6-carboxamide |
| [Cpd 031] | 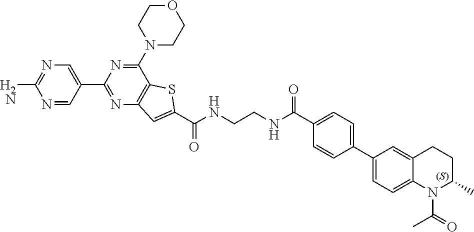 | (S)-N-(2-(4-(1-Acetyl-2-me- thyl-1,2,3,4-tetrahydroquin- olin-6-yl)benzamido)ethyl)- 2-(2-aminopyrimidin-5-yl)- 4-morpholinothieno[3,2- d]pyrimidine-6-carbox- amide |
| [Cpd 032] | 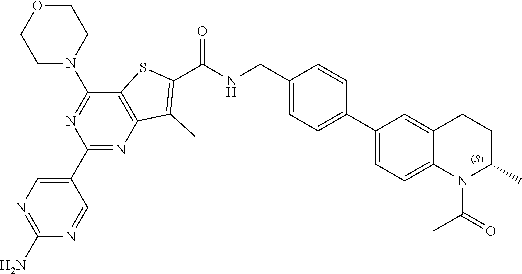 | (S)-N-(4-(1-Acetyl-2-me- thyl-1,2,3,4-tetrahydroquin- olin-6-yl)benzyl)-2-(2-ami- nopyrimidin-5-yl)-7-methyl- 4-morpholinothieno[3,2- d]pyrimidine-6-carbox- amide |
| [Cpd 033] | 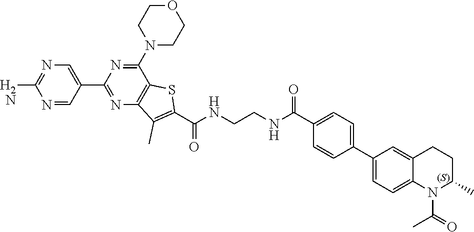 | (S)-N-(2-(4-(1-Acetyl-2-me- thyl-1,2,3,4-tetrahydroquin- olin-6-yl)benzamido)ethyl)- 2-(2-aminopyrimidin-5-yl)- 7-methyl-4-morpho- linothieno[3,2-d]pyrimi- dine-6-carboxamide |
| [Cpd 034] | 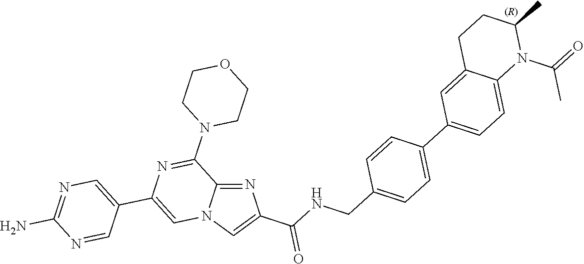 | (R)-N-(4-(1-Acetyl-2-me- thyl-1,2,3,4-tetrahydroquin- olin-6-yl)benzyl)-6-(2-ami- nopyrimidin-5-yl)-8-mor- pholinoimidazo[1,2-a]pyra- zine-2-carboxamide |
| [Cpd 035] | 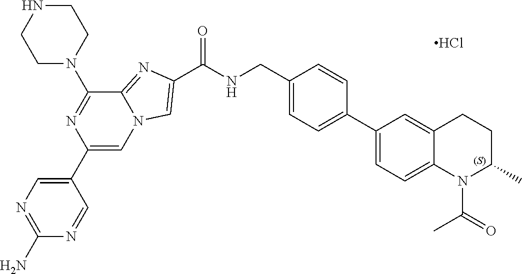 | (S)-N-(4-(1-Acetyl-2-me- thyl-1,2,3,4-tetrahydroquin- olin-6-yl)benzyl)-6-(2-ami- nopyrimidin-5-yl)-8-(piper- azin-1-yl)imidazo[1,2-a]py- razine-2-carboxamide hy- drochloride |
| [Cpd 036] | 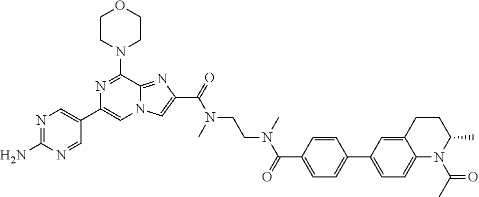 [SMILES: CC(=O)N1c2ccc(-c3ccc(C(=O)N(C)CCN(C)C(=O)c4cn5cc(-c6cnc(N)nc6)nc(N6CCOCC6)c5n4)cc3)cc2CC[C@@H]1C] | (S)-N-(2-(4-(1-Acetyl-2-me- thyl-1,2,3,4-tetrahydroquin- olin-6-yl)-N-methylben- zamido)ethyl)-6-(2-amino- pyrimidin-5-yl)-N-methyl-8- morpholinoimidazo[1,2- a]pyrazine-2-carboxamide |
| [Cpd 037] | 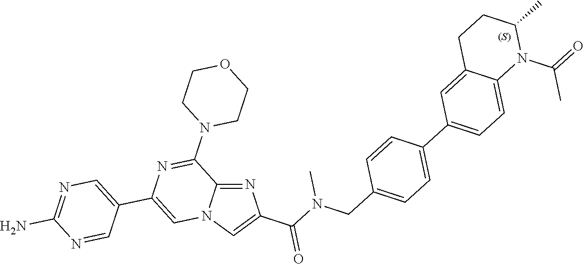 | (S)-N-(4-(1-Acetyl-2-me- thyl-1,2,3,4-tetrahydroquin- olin-6-yl)benzyl)-6-(2-ami- nopyrimidin-5-yl)-N-me- thyl-8-morpholinoimid- azo[1,2-a]pyrazine-2-car- boxamide |
| [Cpd 038] | 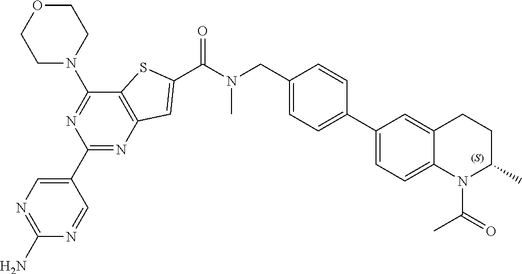 | (S)-N-(4-(1-Acetyl-2-me- thyl-1,2,3,4-tetrahydroquin- olin-6-yl)benzyl)-2-(2-ami- nopyrimidin-5-yl)-N-me- thyl-4-morpho- linothieno[3,2-d]pyrimi- dine-6-carboxamide |
| [Cpd 039] | 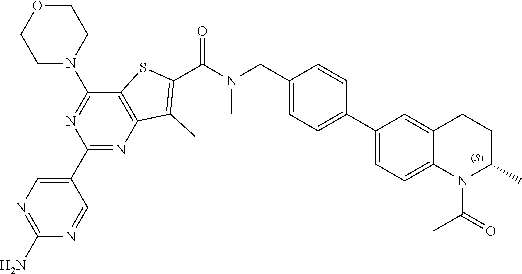 | (S)-N-(4-(1-Acetyl-2-me- thyl-1,2,3,4-tetrahydroquin- olin-6-yl)benzyl)-2-(2-ami- nopyrimidin-5-yl)-N,7-di- methyl-4-morpho- linothieno[3,2-d]pyrimi- dine-6-carboxamide |
| [Cpd 040] | 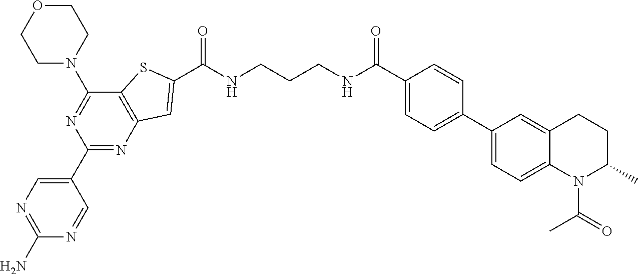 [SMILES: CC(=O)N1c2ccc(-c3ccc(C(=O)NCCCNC(=O)c4cc5nc(-c6cnc(N)nc6)nc(N6CCOCC6)c5s4)cc3)cc2CC[C@@H]1C] | (S)-N-(3-(4-(1-Acetyl-2-me- thyl-1,2,3,4-tetrahydroquin- olin-6-yl)benzamido)pro- pyl)-2-(2-aminopyrimidin- 5-yl)-4-morpho- linothieno[3,2-d]pyrimi- dine-6-carboxamide |
| [Cpd 041] | 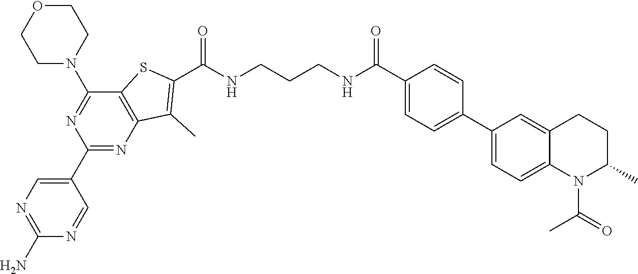 [SMILES: CC(=O)N1c2ccc(-c3ccc(C(=O)NCCCNC(=O)c4sc5c(N6CCOCC6)nc(-c6cnc(N)nc6)nc5c4C)cc3)cc2CC[C@@H]1C] | (S)-N-(3-(4-(1-Acetyl-2-me- thyl-1,2,3,4-tetrahydroquin- olin-6-yl)benzamido)pro- pyl)-2-(2-aminopyrimidin- 5-yl)-7-methyl-4-morpho- linothieno[3,2-d]pyrimi- dine-6-carboxamide |
| [Cpd 042] | 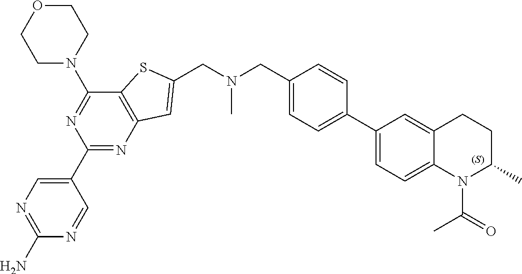 | (S)-1-(6-(4-((((2-(2-Amino- pyrimidin-5-yl)-4-morpho- linothieno[3,2-d]pyrimidin- 6-yl)methyl)(me- thyl)amino)methyl)phenyl)- 2-methyl-3,4-dihydroquino- lin-1(2H)-yl)ethan-1-one |
| [Cpd 043] | 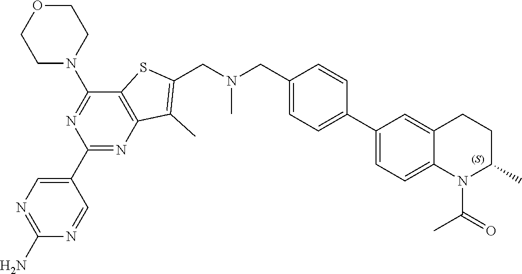 | (S)-1-(6-(4-((((2-(2-Amino- pyrimidin-5-yl)-7-methyl-4- morpholinothieno[3,2-d]py- rimidin-6-yl)methyl)(me- thyl)amino)methyl)phenyl)- 2-methyl-3,4-dihydroquino- lin-1(2H)-yl)ethan-1-one |
| [Cpd 044] | 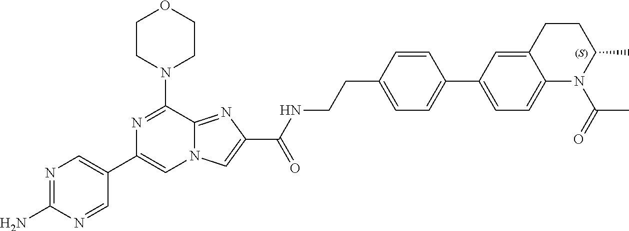 | (S)-N-(4-(1-Acetyl-2-me- thyl-1,2,3,4-tetrahydroquin- olin-6-yl)phenethyl)-6-(2- aminopyrimidin-5-yl)-8- morpholinoimidazo[1,2- a]pyrazine-2-carboxamide |
| [Cpd 045] | 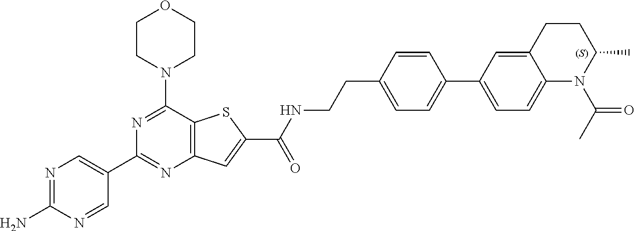 | (S)-N-(4-(1-Acetyl-2-me- thyl-1,2,3,4-tetrahydroquin- olin-6-yl)phenethyl)-2-(2- aminopyrimidin-5-yl)-4- morpholinothieno[3,2-d]py- rimidine-6-carboxamide |
| [Cpd 046] | 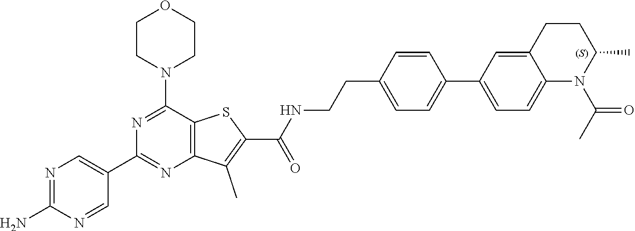 | (S)-N-(4-(1-Acetyl-2-me- thyl-1,2,3,4-tetrahydroquin- olin-6-yl)phenethyl)-2-(2- aminopyrimidin-5-yl)-7-me- thyl-4-morpho- linothieno[3,2-d]pyrimi- dine-6-carboxamide |
| [Cpd 047] | 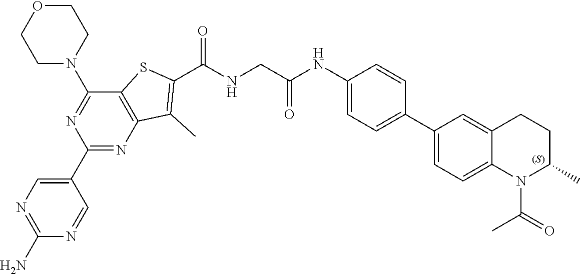 | (S)-N-(2-((4-(1-Acetyl-2- methyl-1,2,3,4-tetrahydro- quinolin-6-yl)phe- nyl)amino)-2-oxoethyl)-2- (2-aminopyrimidin-5-yl)-7- methyl-4-morpho- linothieno[3,2-d]pyrimi- dine-6-carboxamide |
| [Cpd 048] | 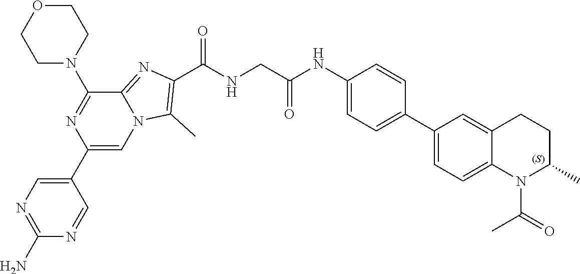 | (S)-N-(2-((4-(1-Acetyl-2- methyl-1,2,3,4-tetrahydro- quinolin-6-yl)phe- nyl)amino)-2-oxoethyl)-6- (2-aminopyrimidin-5-yl)-3- methyl-8-morpholinoimid- azo[1,2-a]pyrazine-2-car- boxamide |
| [Cpd 049] | 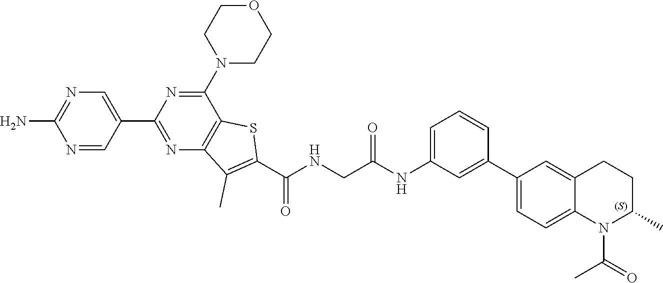 | (S)-N-(2-((3-(1-Acetyl-2- methyl-1,2,3,4-tetrahydro- quinolin-6-yl)phe- nyl)amino)-2-oxoethyl)-2- (2-aminopyrimidin-5-yl)-7- methyl-4-morpho- linothieno[3,2-d]pyrimi- dine-6-carboxamide |
| [Cpd 050] | 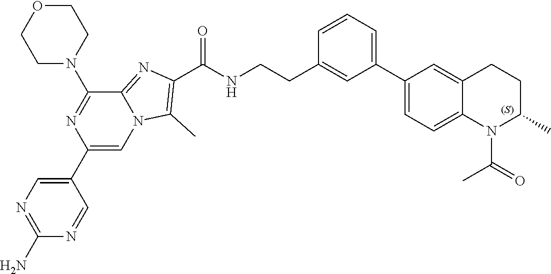 | (S)-N-(3-(1-Acetyl-2-me- thyl-1,2,3,4-tetrahydroquin- olin-6-yl)phenethyl)-6-(2- aminopyrimidin-5-yl)-3-me- thyl-8-morpholinoimid- azo[1,2-a]pyrazine-2-car- boxamide |
| [Cpd 051] | 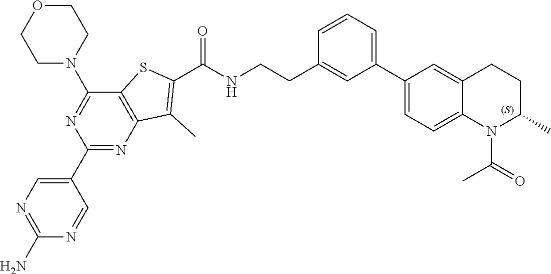 | (S)-N-(3-(1-Acetyl-2-me- thyl-1,2,3,4-tetrahydroquin- olin-6-yl)phenethyl)-2-(2- aminopyrimidin-5-yl)-7-me- thyl-4-morpho- linothieno[3,2-d]pyrimi- dine-6-carboxamide |
| [Cpd 052] | 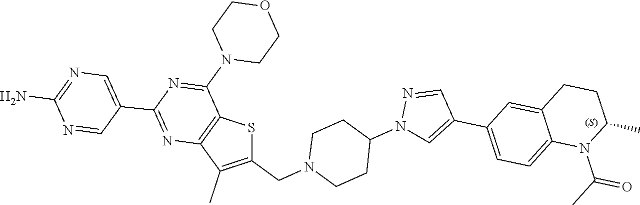 | (S)-1-(6-(1-(1-((2-(2-Ami- nopyrimidin-5-yl)-7-methyl- 4-morpholinothieno[3,2- d]pyrimidin-6-yl)methyl)pi- peridin-4-yl)-1H-pyrazol-4- yl)-2-methyl-3,4-dihydro- quinolin-1(2H)-yl)ethan-1- one |
| [Cpd 053] | 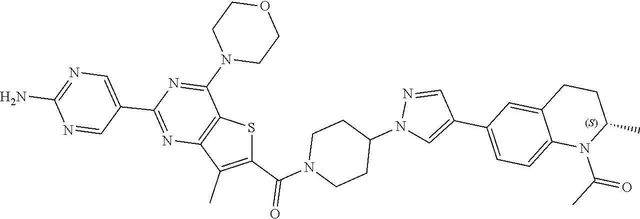 | (S)-1-(6-(1-(1-(2-(2-Amino- pyrimidin-5-yl)-7-methyl-4- morpholinothieno[3,2-d]py- rimidine-6-carbonyl)piperi- din-4-yl)-1H-pyrazol-4-yl)- 2-methyl-3,4-dihydroquino- lin-1(2H)-yl)ethan-1-one |
| [Cpd 054] | 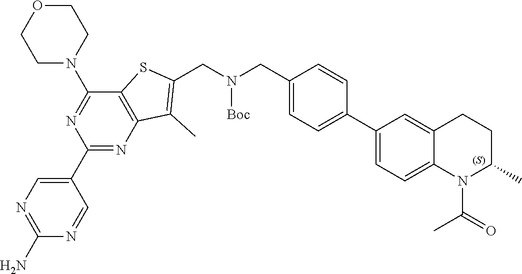 | tert-Butyl (S)-(4-(1-acetyl- 2-methyl-1,2,3,4-tetrahydro- quinolin-6-yl)benzyl)((2-(2- aminopyrimidin-5-yl)-7-me- thyl-4-morpho- linothieno[3,2-d]pyrimidin- 6-yl)methyl)carbamate |
| [Cpd 055] | 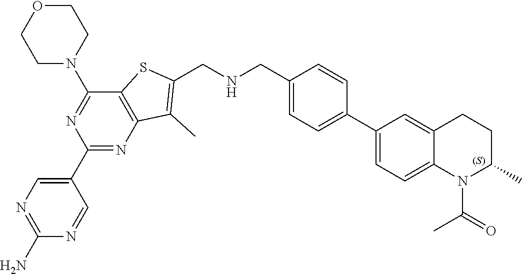 | (S)-1-(6-(4-((((2-(2-Amino- pyrimidin-5-yl)-7-methyl-4- morpholinothieno[3,2-d]py- rimidin-6-yl)me- thyl)amino)methyl)phenyl)- 2-methyl-3,4-dihydroquino- lin-1(2H)-yl)ethan-1-one |
| [Cpd 056] | 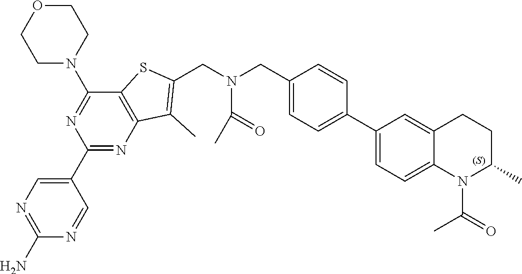 | (S)-N-(4-(1-Acetyl-2-me- thyl-1,2,3,4-tetrahydroquin- olin-6-yl)benzyl)-N-((2-(2- aminopyrimidin-5-yl)-7-me- thyl-4-morpho- linothieno[3,2-d]pyrimidin- 6-yl)methyl)acetamide |
| [Cpd 057] | 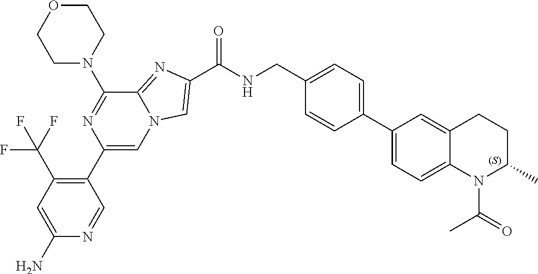 | (S)-N-(4-(1-Acetyl-2-me- thyl-1,2,3,4-tetrahydroquin- olin-6-yl)benzyl)-6-(6- amino-4-(trifluorome- thyl)pyridin-3-yl)-8-mor- pholinoimidazo[1,2-a]pyra- zine-2-carboxamide |
| [Cpd 058] | 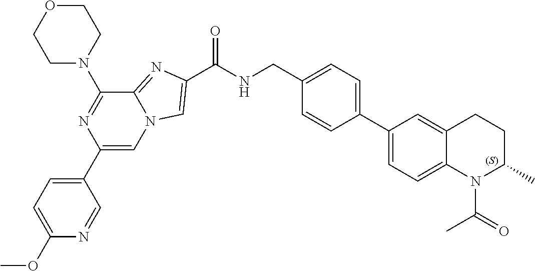 | (S)-N-(4-(1-Acetyl-2-me- thyl-1,2,3,4-tetrahydroquin- olin-6-yl)benzyl)-6-(6-meth- oxypyridin-3-yl)-8-morpho- linoimidazo[1,2-a]pyrazine- 2-carboxamide |
| [Cpd 059] | 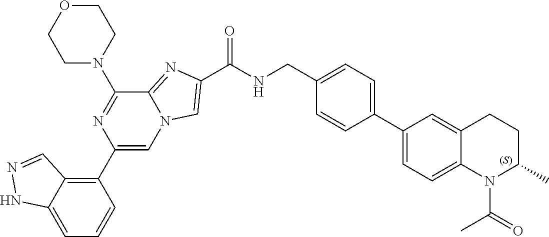 | (S)-N-(4-(1-Acetyl-2-me- thyl-1,2,3,4-tetrahydroquin- olin-6-yl)benzyl)-6-(1H-in- dazol-4-yl)-8-morpho- linoimidazo[1,2-a]pyrazine- 2-carboxamide |
| [Cpd 060] | 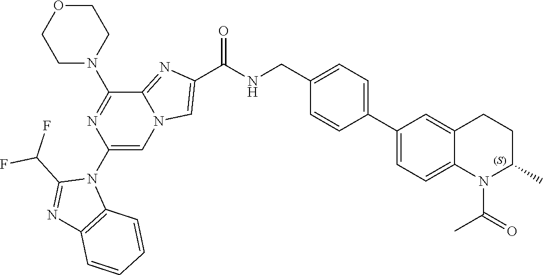 | (S)-N-(4-(1-Acetyl-2-me- thyl-1,2,3,4-tetrahydroquin- olin-6-yl)benzyl)-6-(2- (difluoromethyl)-1H- benzo[d]imidazol-1-yl)-8- morpholinoimidazo[1,2- a]pyrazine-2-carboxamide |
| [Cpd 061] | 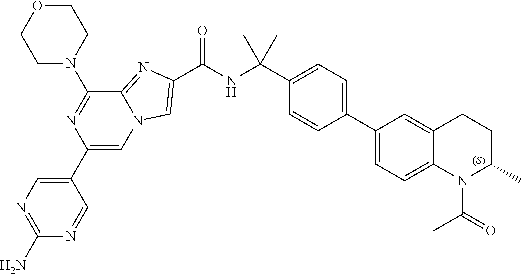 | (S)-N-(2-(4-(1-Acetyl-2-me- thyl-1,2,3,4-tetrahydroquin- olin-6-yl)phenyl)propan-2- yl)-6-(2-aminopyrimidin-5- yl)-8-morpholinoimid- azo[1,2-a]pyrazine-2-car- boxamide |
| [Cpd 062] | 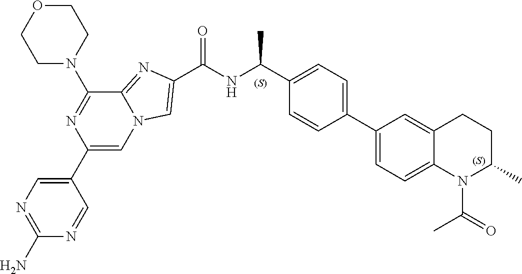 | N-((S)-1-(4-((S)-1-Acetyl-2- methyl-1,2,3,4-tetrahydro- quinolin-6-yl)phenyl)ethyl)- 6-(2-aminopyrimidin-5-y])- 8-morpholinoimidazo[1,2- a]pyrazine-2-carboxamide |
| [Cpd 063] | 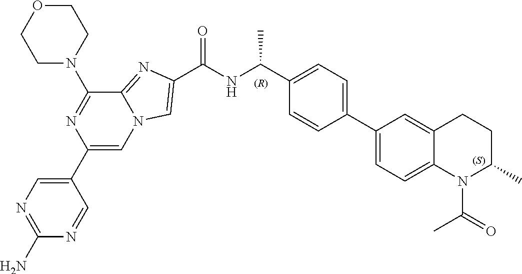 | N-((R)-1-(4-((S)-1-Acetyl- 2-methyl-1,2,3,4-tetrahydro- quinolin-6-yl)phenyl)ethyl)- 6-(2-aminopyrimidin-5-yl)- 8-morpholinoimidazo[1,2- a]pyrazine-2-carboxamide |
| [Cpd 064] | 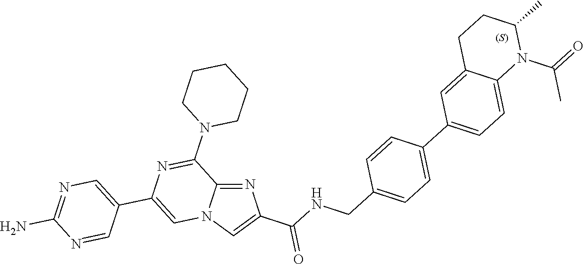 | (S)-N-(4-(1-Acetyl-2-me- thyl-1,2,3,4-tetrahydroquin- olin-6-yl)benzyl)-6-(2-ami- nopyrimidin-5-yl)-8-(piperi- din-1-yl)imidazo[1,2-a]py- razine-2-carboxamide |
| [Cpd 065] | 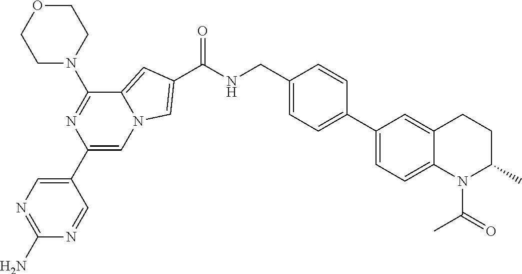 [SMILES: CC(=O)N1c2ccc(-c3ccc(CNC(=O)c4cc5c(N6CCOCC6)nc(-c6cnc(N)nc6)cn5c4)cc3)cc2CC[C@@H]1C] | (S)-N-(4-(1-Acetyl-2-me- thyl-1,2,3,4-tetrahydroquin- olin-6-yl)benzyl)-2-(2-ami- nopyrimidin-5-yl)-4-mor- pholinopyrrolo[2,1- f][1,2,4]triazine-6-carbox- amide |
| [Cpd 066] | 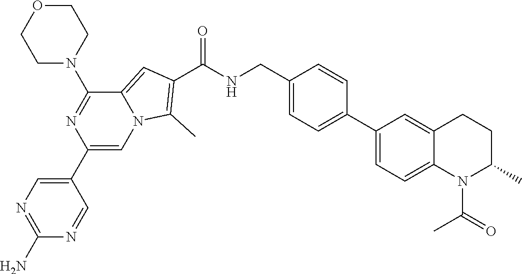 [SMILES: CC(=O)N1c2ccc(-c3ccc(CNC(=O)c4cc5c(N6CCOCC6)nc(-c6cnc(N)nc6)cn5c4C)cc3)cc2CC[C@@H]1C] | (S)-N-(4-(1-Acetyl-2-me- thyl-1,2,3,4-tetrahydroquin- olin-6-yl)benzyl)-2-(2-ami- nopyrimidin-5-yl)-7-methyl- 4-morpholinopyrrolo[2,1- f][1,2,4]triazine-6-carbox- amide |
| [Cpd 067] | 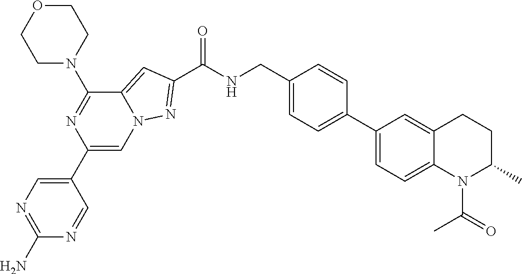 [SMILES: CC(=O)N1c2ccc(-c3ccc(CNC(=O)c4cc5c(N6CCOCC6)nc(-c6cnc(N)nc6)cn5n4)cc3)cc2CC[C@@H]1C] | (S)-N-(4-(1-Acetyl-2-me- thyl-1,2,3,4-tetrahydroquin- olin-6-yl)benzyl)-6-(2-ami- nopyrimidin-5-yl)-4-mor- pholinopyrazolo[1,5-a]pyra- zine-2-carboxamide |
| [Cpd 068] | 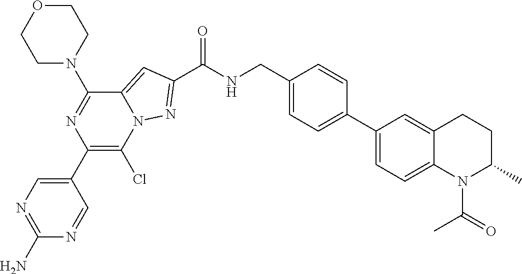 [SMILES: CC(=O)N1c2ccc(-c3ccc(CNC(=O)c4cc5c(N6CCOCC6)nc(-c6cnc(N)nc6)c(Cl)n5n4)cc3)cc2CC[C@@H]1C] | (S)-N-(4-(1-Acetyl-2-me- thyl-1,2,3,4-tetrahydroquin- olin-6-yl)benzyl)-6-(2-ami- nopyrimidin-5-yl)-7-chloro- 4-morpholinopyrazolo[1,5- a]pyrazine-2-carboxamide |
| [Cpd 069] | 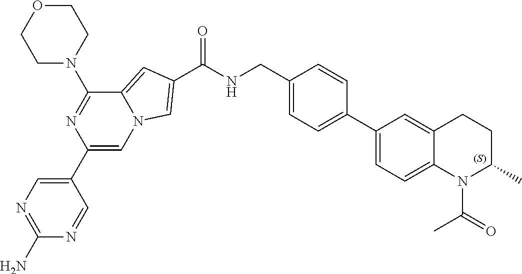 | (S)-N-(4-(1-Acetyl-2-me- thyl-1,2,3,4-tetrahydroquin- olin-6-yl)benzyl)-3-(2-ami- nopyrimidin-5-yl)-1-mor- pholinopyrrolo[1,2-a]pyra- zine-7-carboxamide |
| [Cpd 070] | 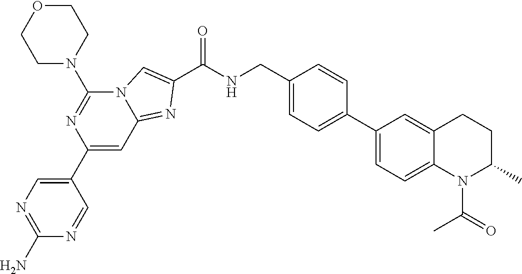 [SMILES: CC(=O)N1c2ccc(-c3ccc(CNC(=O)c4cn5c(N6CCOCC6)nc(-c6cnc(N)nc6)cc5n4)cc3)cc2CC[C@@H]1C] | (S)-N-(4-(1-Acetyl-2-me- thyl-1,2,3,4-tetrahydroquin- olin-6-yl)benzyl)-7-(2-ami- nopyrimidin-5-yl)-5-mor- pholinoimidazo[1,2-c]py- rimidine-2-carboxamide |
| [Cpd 071] | 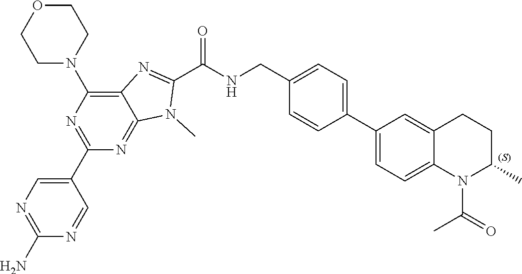 | (S)-N-(4-(1-Acetyl-2-me- thyl-1,2,3,4-tetrahydroquin- olin-6-yl)benzyl)-2-(2-ami- nopyrimidin-5-yl)-9-methyl- 6-morpholino-9H-purine-8- carboxamide |
| [Cpd 072] | 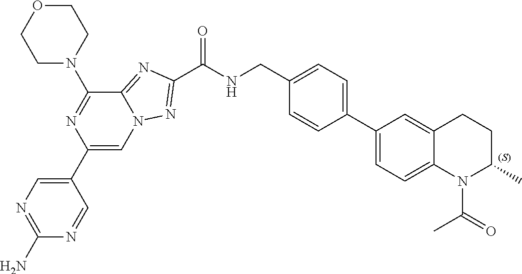 | (S)-N-(4-(1-Acetyl-2-me- thyl-1,2,3,4-tetrahydroquin- olin-6-yl)benzyl)-6-(2-ami- nopyrimidin-5-yl)-8-mor- pholino-[1,2,4]triazolo[1,5- a]pyrazine-2-carboxamide |
| [Cpd 073] | 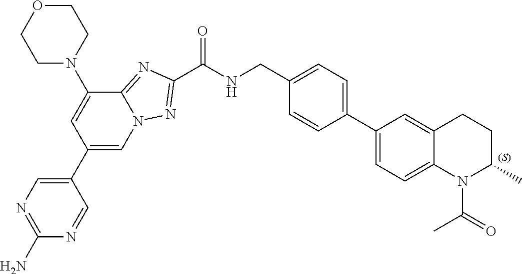 | (S)-N-(4-(1-Acetyl-2-me- thyl-1,2,3,4-tetrahydroquin- olin-6-yl)benzyl)-6-(2-ami- nopyrimidin-5-yl)-8-mor- pholino-[1,2,4]triazolo[1,5- a]pyridine-2-carboxamide |
| [Cpd 074] | 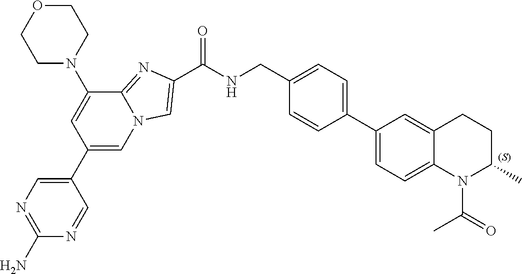 | (S)-N-(4-(1-Acetyl-2-me- thyl-1,2,3,4-tetrahydroquin- olin-6-yl)benzyl)-6-(2-ami- nopyrimidin-5-yl)-8-mor- pholinoimidazo[1,2-a]pyri- dine-2-carboxamide |
| [Cpd 075] | 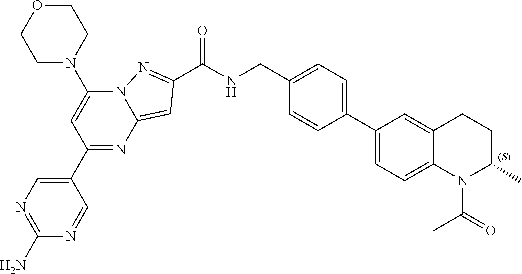 | (S)-N-(4-(1-Acetyl-2-me- thyl-1,2,3,4-tetrahydroquin- olin-6-yl)benzyl)-5-(2-ami- nopyrimidin-5-yl)-7-mor- pholinopyrazolo[1,5-a]py- rimidine-2-carboxamide |
| [Cpd 076] | 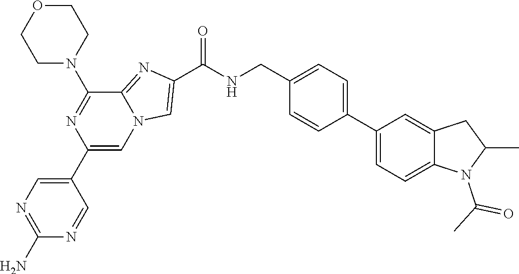 [SMILES: CC(=O)N1c2ccc(-c3ccc(CNC(=O)c4cn5cc(-c6cnc(N)nc6)nc(N6CCOCC6)c5n4)cc3)cc2CC1C] | N-(4-(1-Acetyl-2-methylin- dolin-5-yl)benzyl)-6-(2-ami- nopyrimidin-5-yl)-8-mor- pholinoimidazo[1,2-a]pyra- zine-2-carboxamide |
| [Cpd 077] | 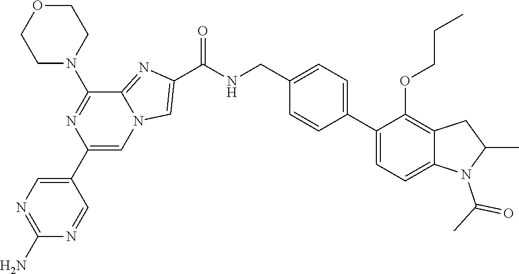 [SMILES: CCCOc1c(-c2ccc(CNC(=O)c3cn4cc(-c5cnc(N)nc5)nc(N5CCOCC5)c4n3)cc2)ccc2c1CC(C)N2C(C)=O] | N-(4-(1-Acetyl-2-methyl-4- propoxyindolin-5-yl)ben- zyl)-6-(2-aminopyrimidin-5- yl)-8-morpholinoimid- azo[1,2-a]pyrazine-2-car- boxamide |
| [Cpd 078] | 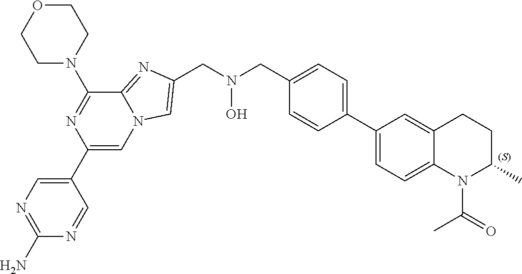 | (S)-1-(6-(4-((((6-(2-Amino- pyrimidin-5-yl)-8-morpho- linoimidazo[1,2-a]pyrazin- 2-yl)methyl)(hy- droxy)amino)methyl)phe- nyl)-2-methyl-3,4-dihydro- quinolin-1(2H)-yl)ethan-1- one |

### Abbreviations

Å Angstrom
uL Microliter
abs. Absolute
ACN Acetonitrile
AcOH Acetic acid
aq. Aqueous
bicarb. /NaHCO₃Sodium bicarbonate/Sodium hydrogen carbonate
Boc tert-Butyloxycarbonyl
BOP Benzotriazol-1-yloxytris(dimethylamino)phosphonium hexafluorophosphate
n-BuLi n-Butyllithium
tBuXPhos 2-Di-tert-butylphosphino-2′,4′,6′-triisopropylbiphenyl
° C. degree Celsius
CHCl₃ Chloroform
¹³C NMR Carbon-13 nuclear magnetic resonance
Conc. Concentrated
m-CPB A meta-Chloroperoxybenzoic acid
Cs₂CO₃ Cesium carbonate
min Minute(s)
d Day(s)
DBU 1,8-Diazabicyclo[5.4.0]undec-7-ene
DCM Dichloromethane
DCE Dichloroethane
de Diastereomeric excess
DIBAL Diisobutylaluminium hydride
DIPEA N,N-Diisopropylethylamine
DME Dimethoxyethane
DMF N,N-Dimethylformamide
DMSO Dimethylsulfoxide
EA Ethyl acetate
EDC 1-Ethyl-3-(3-dimethylaminopropyl)carbodiimide
ee Enantiomeric excess
ESI Electrospray ionization
EtOH Ethanol
HCl Hydrochloride
HCTU O-(1H-6-Chlorobenzotriazole-1-yl)-1,1,3,3-tetramethyluronium hexafluorophosphate
¹H NMR Proton nuclear magnetic resonance
HOBT Hydroxybenzotriazole
HPLC High performance liquid chromatography
hr Hour(s)
HRMS High Resolution
H₂SO₄ Sulfuric acid
in vacuo In a vacuum
K₂CO₃ Potassium carbonate
KOAc Potassium acetate
KOCN Potassium cyanate
LCMS Liquid chromatography-mass spectrometry
LDA Lithium diisopropylamide
LiHMDS Lithium bis(trimethylsilyl)amide
LiOH Lithium hydroxide
MeOH Methanol
MgSO₄ Magnesium sulfate
mL Milliliter
Mn(OAc)₃ Manganese (III) acetate
MS Molecular seives
MSH O-(Mesitylenesulfonyl)hydroxylamine
m/z Mass to charge ratio
N₂ Dinitrogen/nitrogen gas
NaBH₄ Sodium borohydride
NaOEt Sodium ethoxide
NaCNB H₃ Sodium cyanoborohydride
Na₂CO₃ Sodium carbonate
NaN₃ Sodium azide
NaOAc Sodium acetate
Na(OAc)₃BH Sodium triacetoxyborohydride
NaOH Sodium hydroxide
NBS N-Bromosuccimide
NCS N-Chlorosuccimide
NH₄Cl Ammonium chloride
NMR Nuclear magnetic resonance
o.n Overnight
PBr₃ Phosphorus tribromide
PE Petroleum ether/petroleum benzine/petroleum spirit
prep Preparative
Rf Retention factor
r.t Room temperature
Pd/C Palladium on carbon
PdCl₂(PPh₃)₂ Bids(triphenylphosphine)palladium dichloride
Pd₂(dba)₃ Tris(dibenzylideneacetone)dipalladium
Pd(dppf)Cl₂ (1,1′-Bis(diphenylphosphino)ferrocene)palladium(II) dichloride
POCl₃ Phosphorus oxychloride
p-tol para-Toluene
PyClock 6-Chloro-benzotriazole-1-yloxy-tris-pyrrolidinophosphonium hexafluorophosphate
sat. Saturated
TEA Triethylamine
TFA Trifluoroacetic acid
THE Tetrahydrofuran
TLC Thin layer chromatography
TMEDA Tetramethylethylenediamine
TMSOTf Trimethylsilyl trifluoromethanesulfonate
XPhos Pd G1 (2-Dicyclohexylphosphino-2′,4′,6′-triisopropyl-1,1′-biphenyl)[2-(2-aminoethyl)phenyl)]palladium(II) chloride

### Synthesis of Dual Inhibitor Compounds

### Synthesis of Racemic Precursors

Scheme 1 details the synthesis of the left-hand side of the compounds of Formula (I):

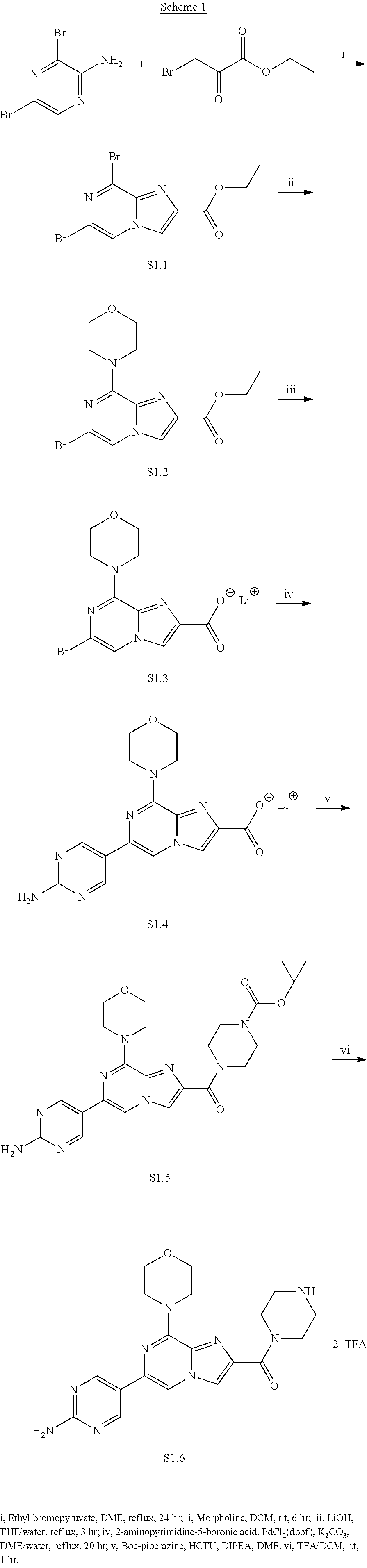

Scheme 2 details the attachment of the linker to the right-hand side of the compounds of Formula (I):

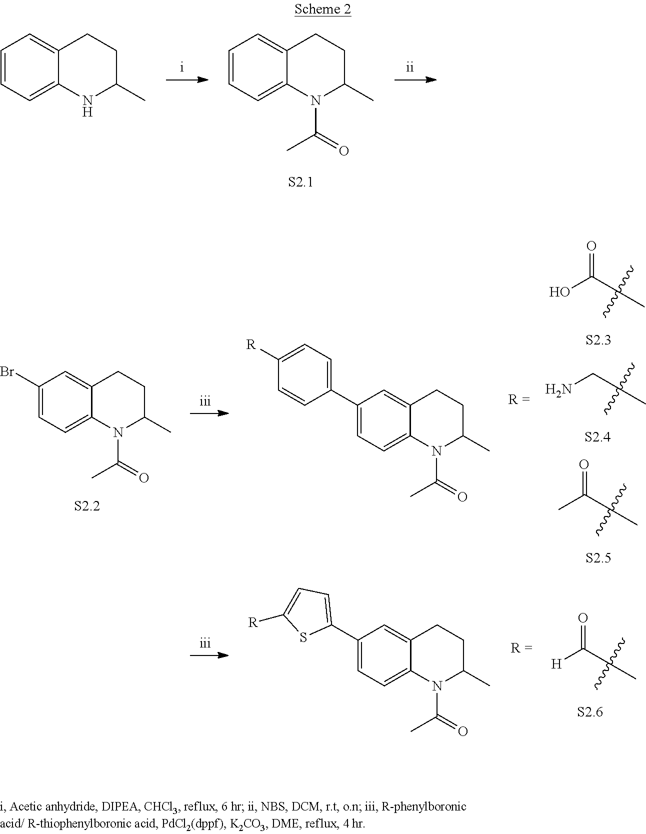

Scheme 3 details the details the attachment of the linker to the right-hand side of the compounds of Formula (I):

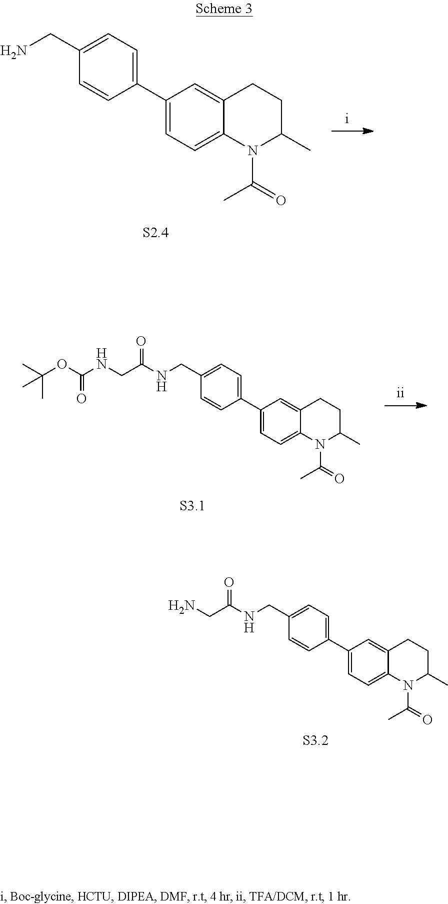

Scheme 4 details the attachment of the linker to the right-hand side of the compounds of Formula (I):

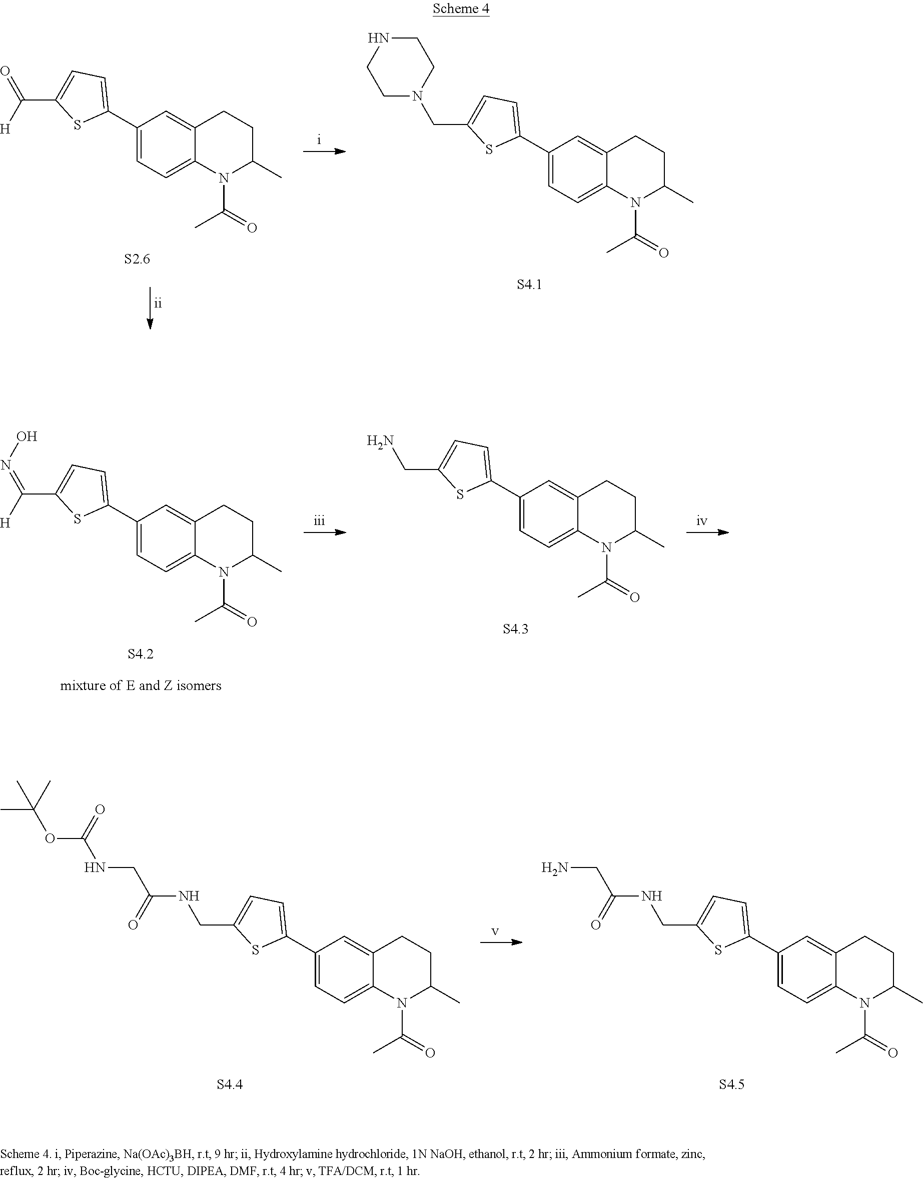 [SMILES: CC(=O)N1c2ccc(-c3ccc(C=O)s3)cc2CCC1C ; CC(=O)N1c2ccc(-c3ccc(CN4CCNCC4)s3)cc2CCC1C ; CC(=O)N1c2ccc(-c3ccc(/C=N\O)s3)cc2CCC1C ; CC(=O)N1c2ccc(-c3ccc(CN)s3)cc2CCC1C ; CC(=O)N1c2ccc(-c3ccc(CNC(=O)CNC(=O)OC(C)(C)C)s3)cc2CCC1C ; CC(=O)N1c2ccc(-c3ccc(CNC(=O)CN)s3)cc2CCC1C]

Scheme 5 details the attachment of the linker to the right-hand side of the compounds of Formula (I):

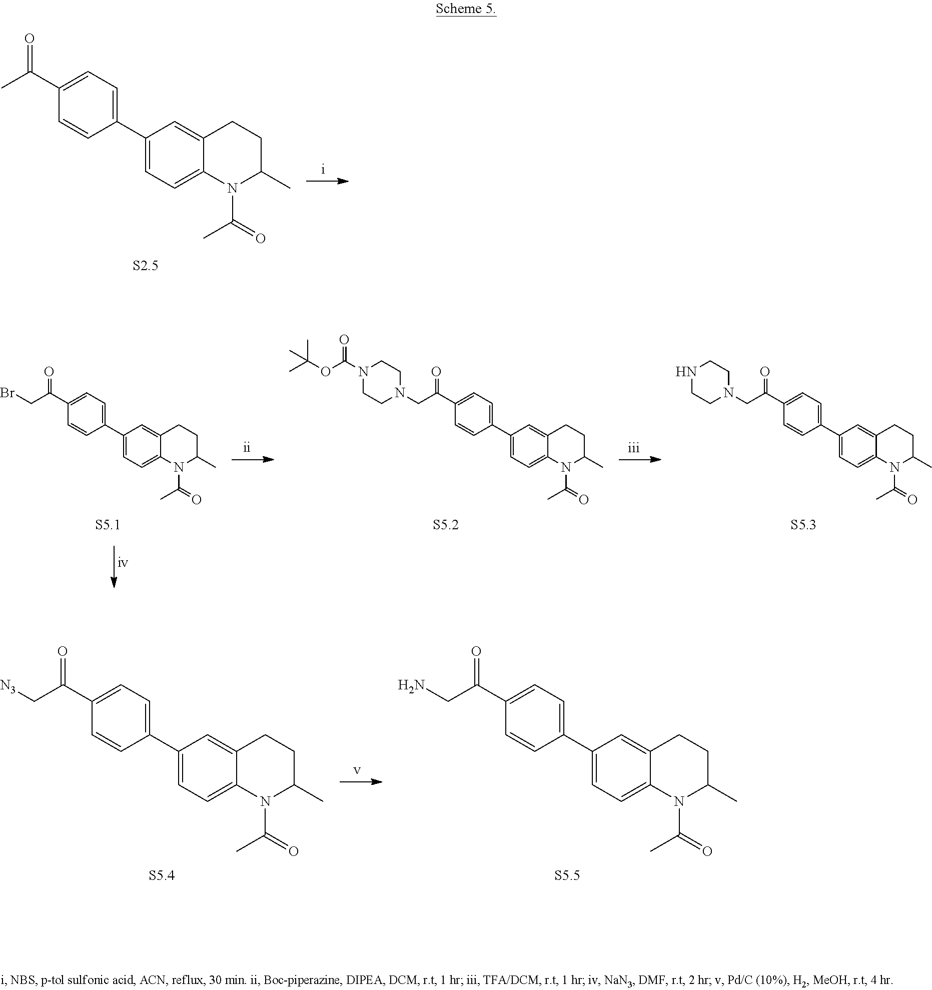 [SMILES: CC(=O)c1ccc(-c2ccc3c(c2)CCC(C)N3C(C)=O)cc1 ; CC(=O)N1c2ccc(-c3ccc(C(=O)CBr)cc3)cc2CCC1C ; CC(=O)N1c2ccc(-c3ccc(C(=O)CN4CCN(C(=O)OC(C)(C)C)CC4)cc3)cc2CCC1C ; CC(=O)N1c2ccc(-c3ccc(C(=O)CN4CCNCC4)cc3)cc2CCC1C ; CC(=O)N1c2ccc(-c3ccc(C(=O)CN=[N+]=[N-])cc3)cc2CCC1C ; CC(=O)N1c2ccc(-c3ccc(C(=O)CN)cc3)cc2CCC1C]

Synthesis of Compounds 001-009

Scheme 6 details the attachment of the left-hand side plus the linker of the compounds of Formula (I) to the right-hand side:

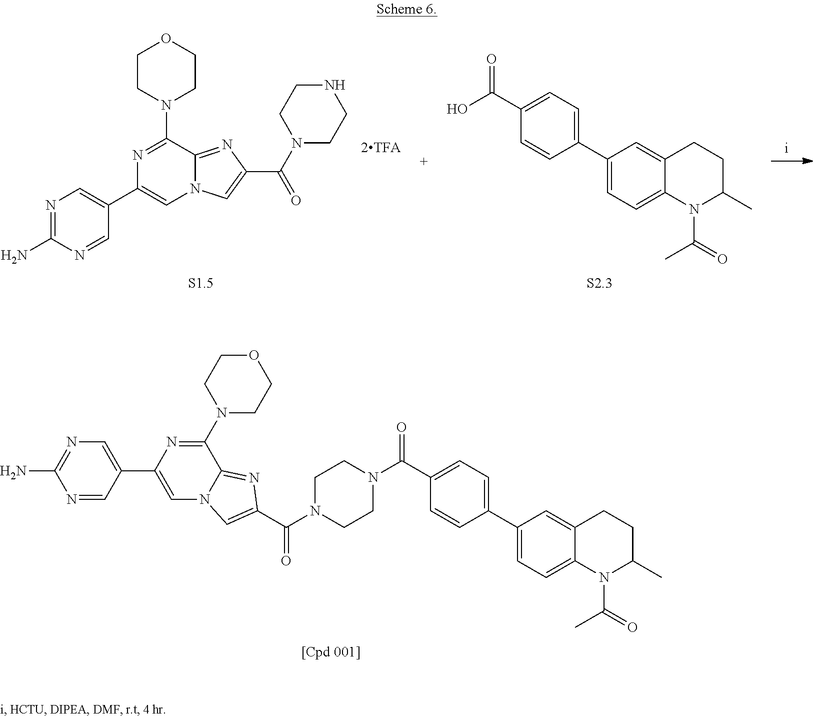 [SMILES: Nc1ncc(-c2cn3cc(C(=O)N4CCNCC4)nc3c(N3CCOCC3)n2)cn1 ; CC(=O)N1c2ccc(-c3ccc(C(=O)O)cc3)cc2CCC1C ; CC(=O)N1c2ccc(-c3ccc(C(=O)N4CCN(C(=O)c5cn6cc(-c7cnc(N)nc7)nc(N7CCOCC7)c6n5)CC4)cc3)cc2CCC1C]

Scheme 7 details the attachment of the right-hand side via the linker of the compounds of Formula (I) to the left-hand side:

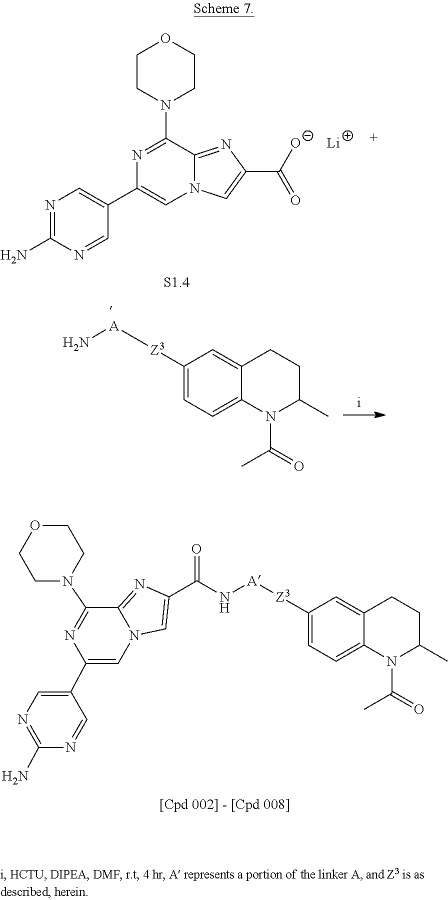

Compounds Cpd 002, Cpd 003, Cpd 004, Cpd 005, Cpd 006, Cpd 007, and Cpd 008 were synthesised according to Scheme 7.

Scheme 8 details the synthesis of Cpd 009:

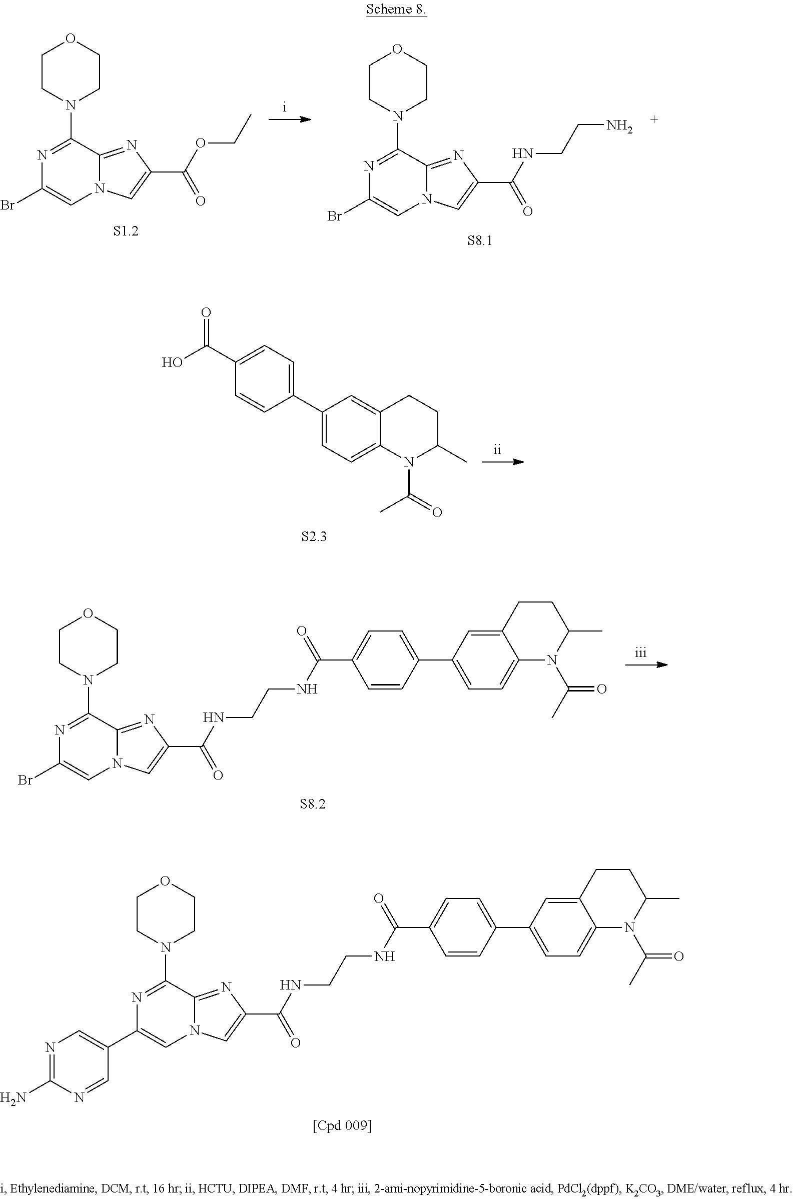 [SMILES: CCOC(=O)c1cn2cc(Br)nc(N3CCOCC3)c2n1 ; NCCNC(=O)c1cn2cc(Br)nc(N3CCOCC3)c2n1 ; CC(=O)N1c2ccc(-c3ccc(C(=O)O)cc3)cc2CCC1C ; CC(=O)N1c2ccc(-c3ccc(C(=O)NCCNC(=O)c4cn5cc(Br)nc(N6CCOCC6)c5n4)cc3)cc2CCC1C ; CC(=O)N1c2ccc(-c3ccc(C(=O)NCCNC(=O)c4cn5cc(-c6cnc(N)nc6)nc(N6CCOCC6)c5n4)cc3)cc2CCC1C]

### Synthesis of Enantiomeric Precursors

Scheme 9 details functionalisation of the right-hand side of compounds of Formula (I):

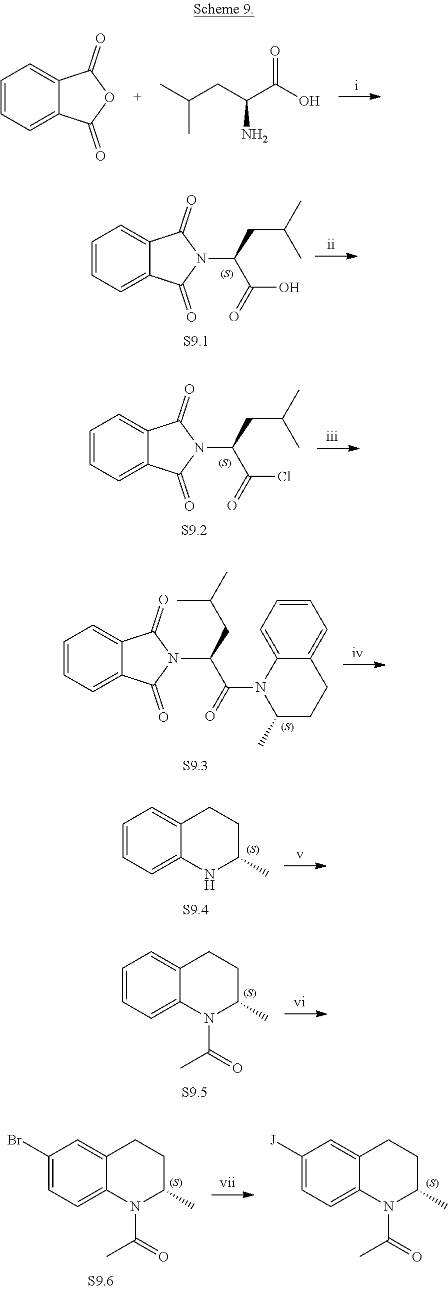

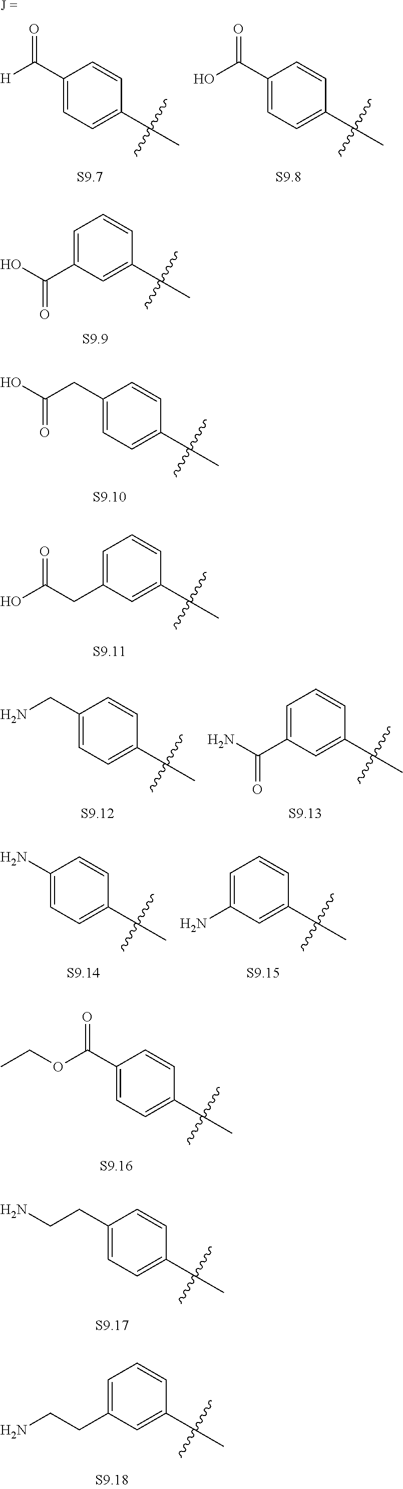

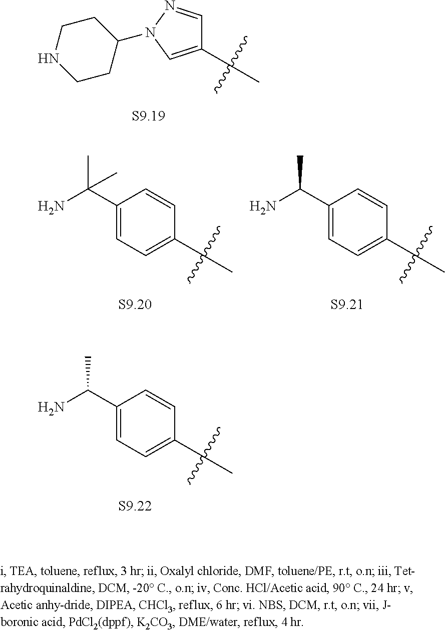

Scheme 10 details attachment of the linker to the right-hand side of compounds of Formula (I):

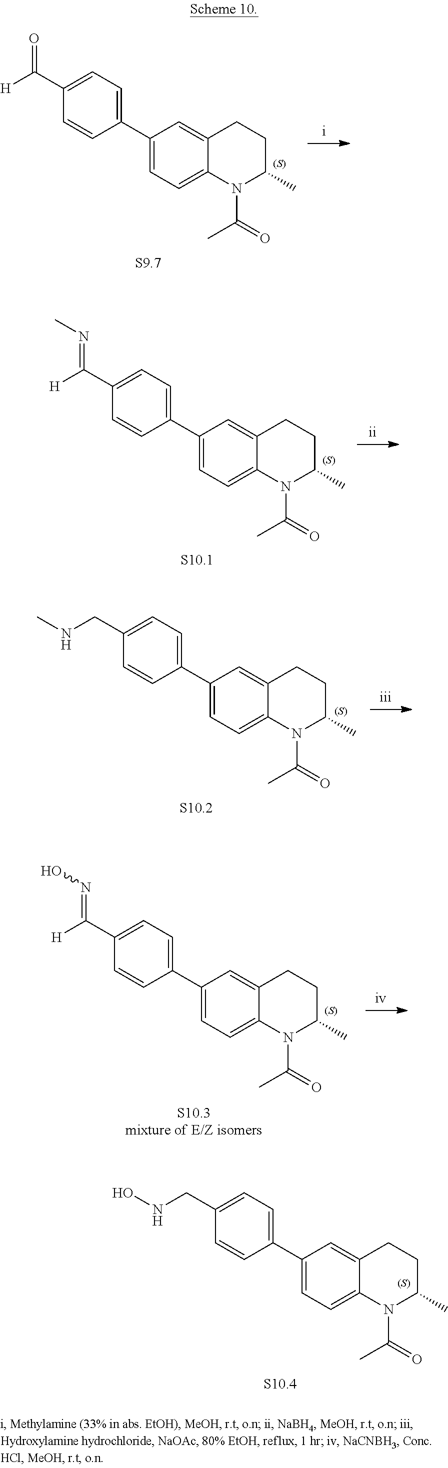

Scheme 11 details attachment of the linker to the right-hand side of the compound of Formula (I):

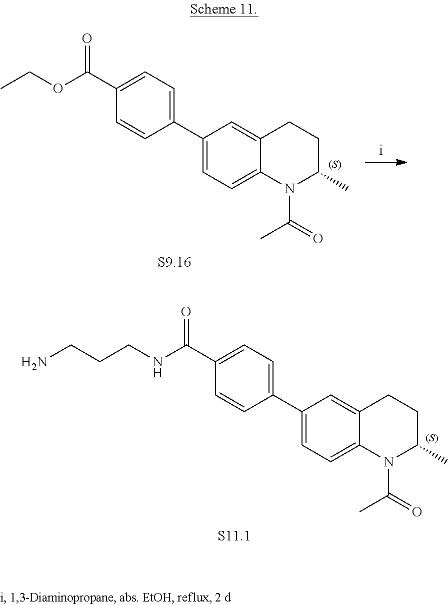

Scheme 12 details attachment of the linker to the right-hand side of the compound of Formula (I):

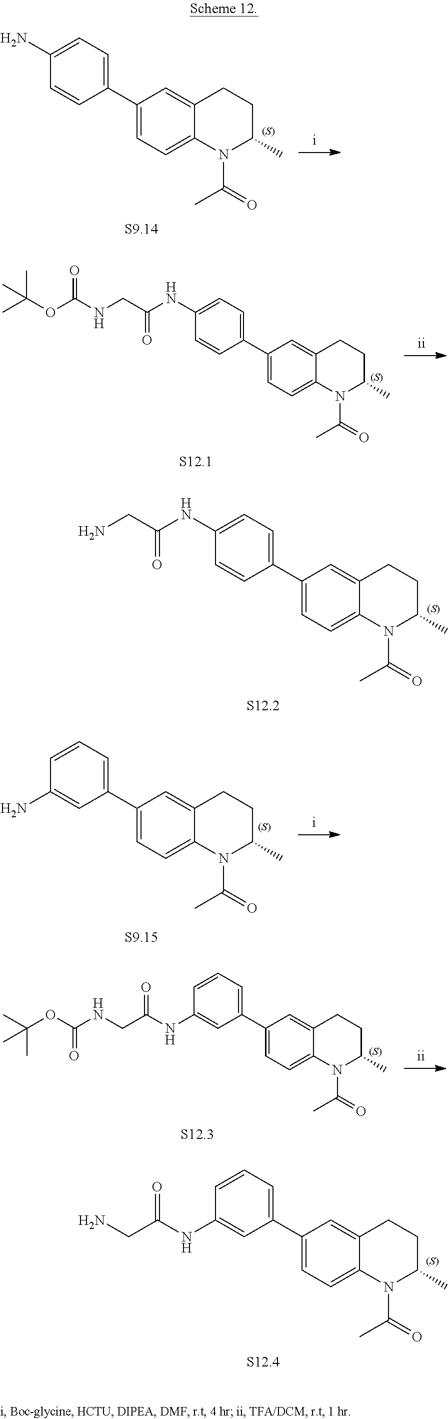

Scheme 13 details functionalisation of, and attachment of the linker to, the left-hand side of compounds of Formula (I):

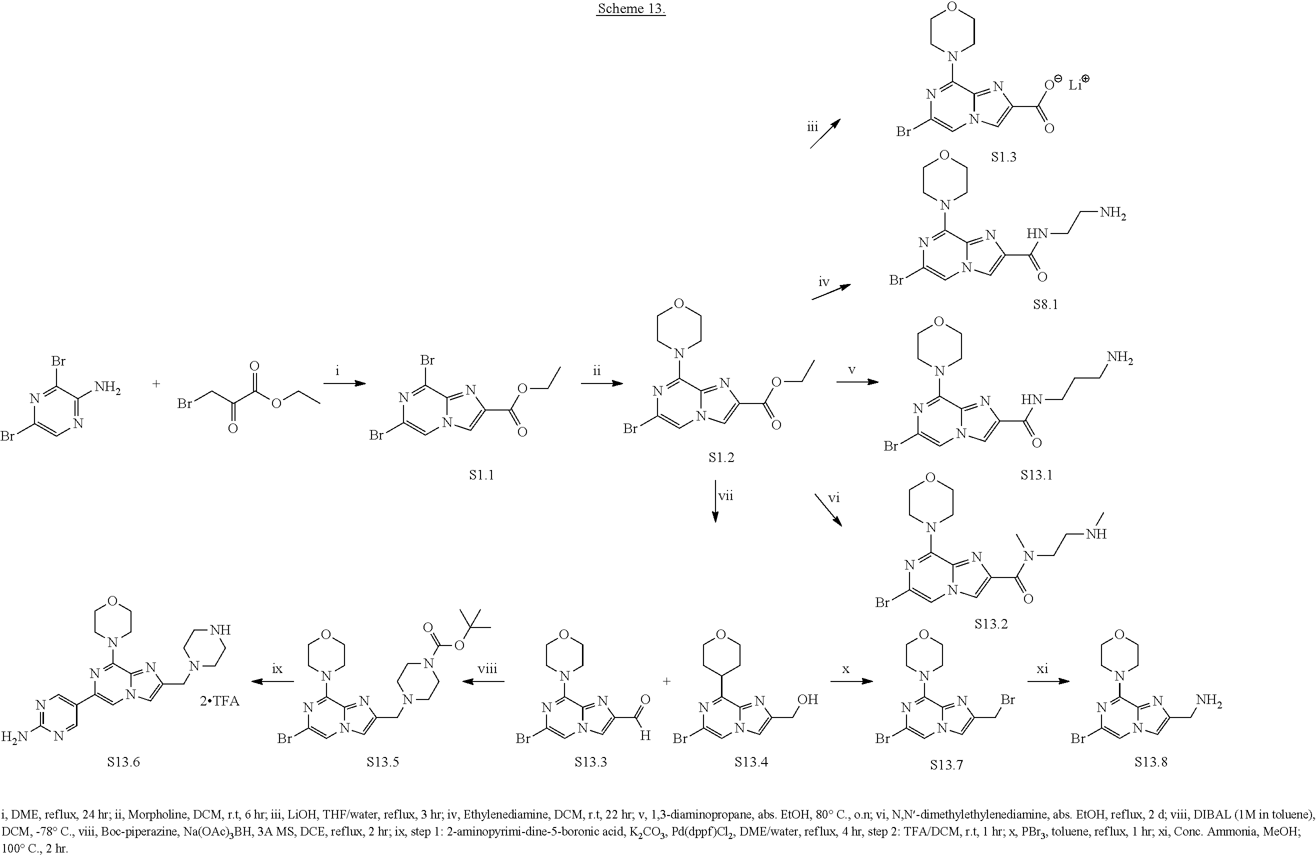

Scheme 14 details functionalisation of, and attachment of, the linker to the left-hand side of the compounds of Formula (I):

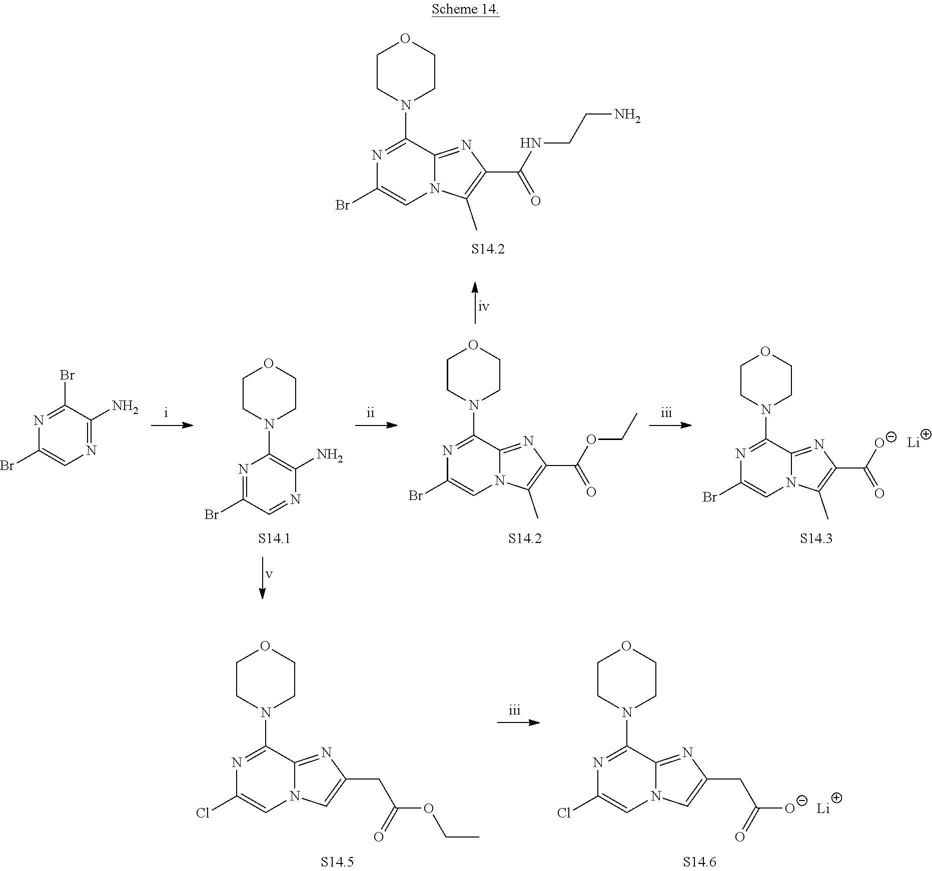

Scheme 15 details functionalisation of, and attachment of, the linker to the left-hand side of the compound of Formula (I):

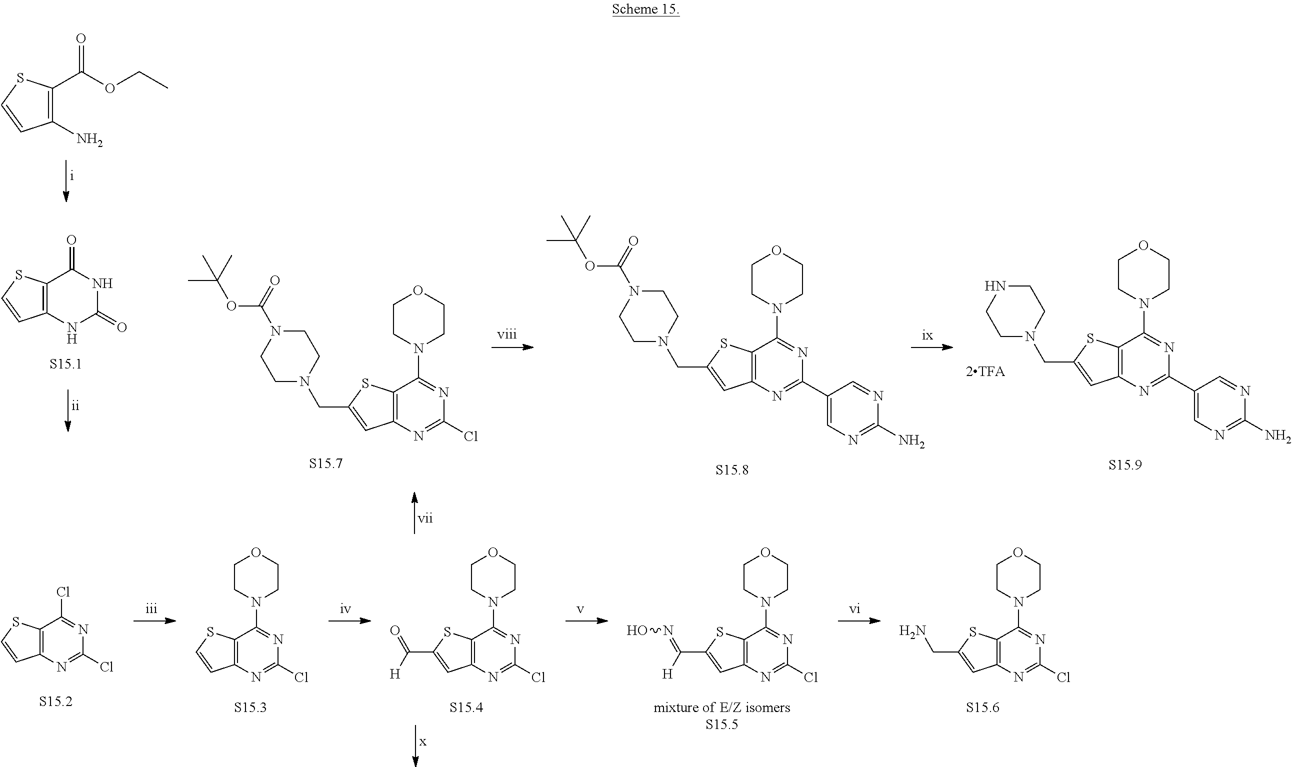

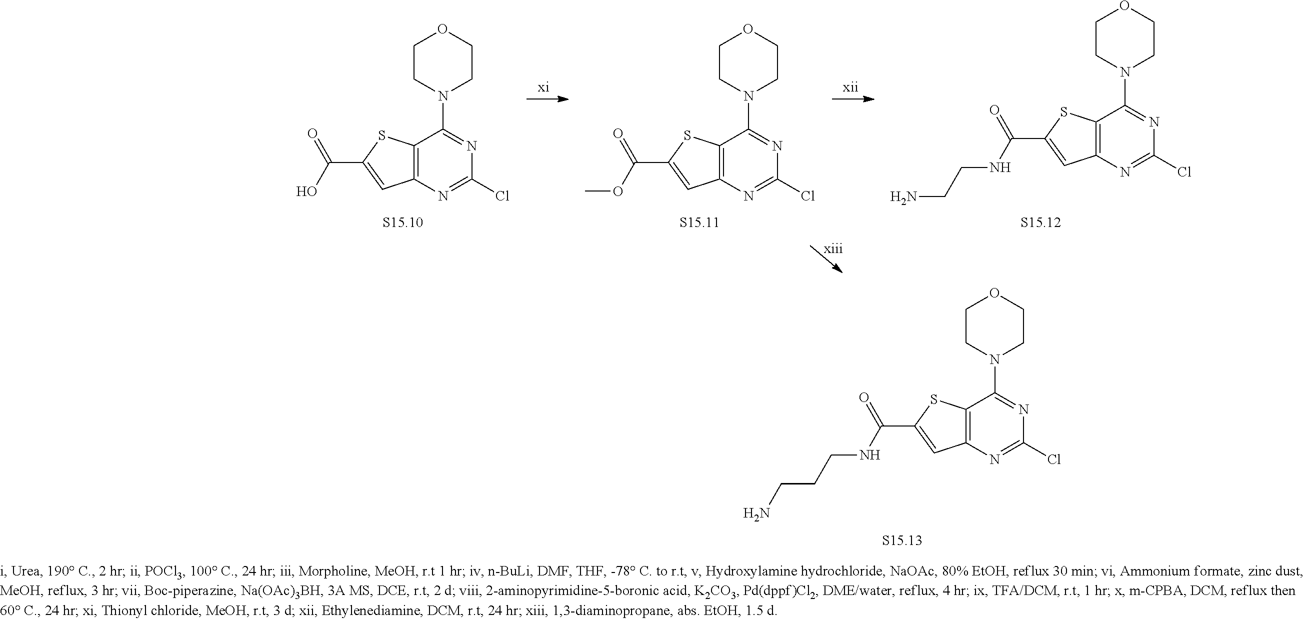 [SMILES: O=C(O)c1cc2nc(Cl)nc(N3CCOCC3)c2s1 ; COC(=O)c1cc2nc(Cl)nc(N3CCOCC3)c2s1 ; NCCNC(=O)c1cc2nc(Cl)nc(N3CCOCC3)c2s1 ; NCCCNC(=O)c1cc2nc(Cl)nc(N3CCOCC3)c2s1]

Scheme 16 details functionalisation of, and attachment of, the linker to the left-hand side of the compound of Formula (I):

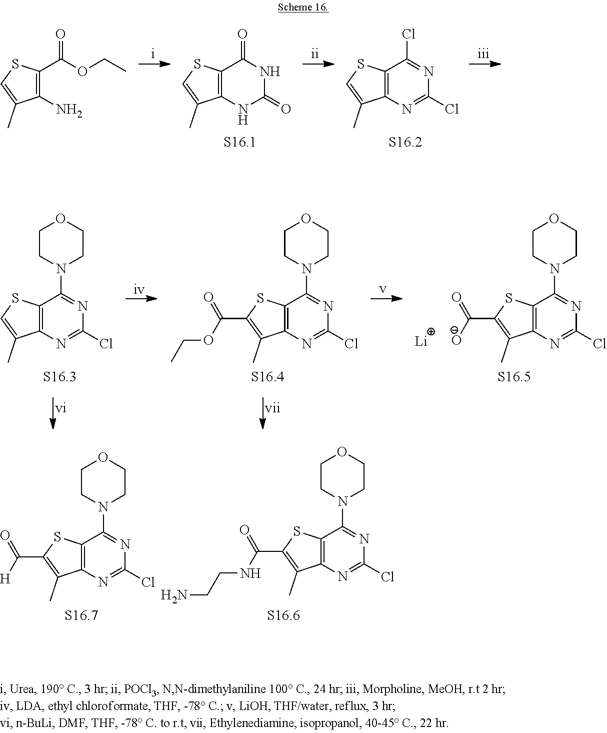

### Preparation of Compounds 010-063 and 078

Scheme 17 details coupling of the left-hand side of compounds of Formula (I) with the right-hand side of the compounds of Formula (I), via the linker:

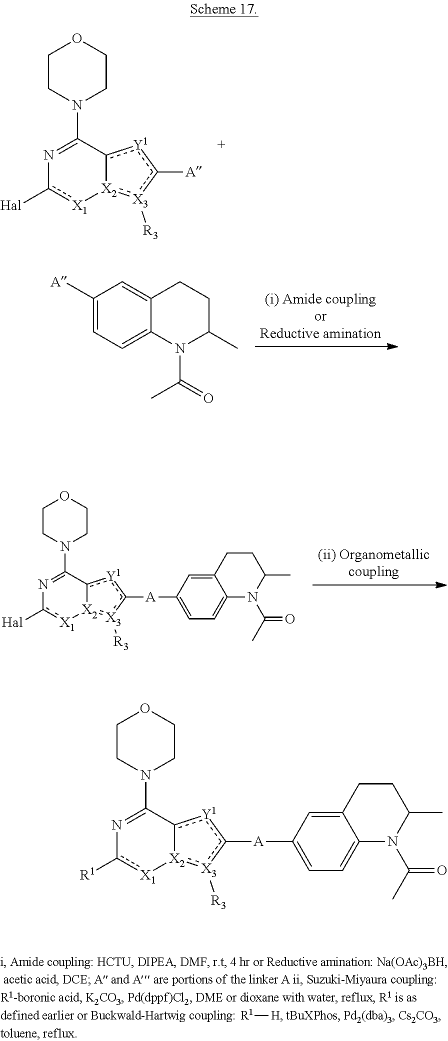

Compounds Cpd 010-013, 016-027, 030-034, 036-041, 044-053, and 057-063 were synthesised according to Scheme 17.

Scheme 18 details coupling of the left-hand side of compounds of Formula (I) with the right-hand side of the compounds of Formula (I), via the linker:

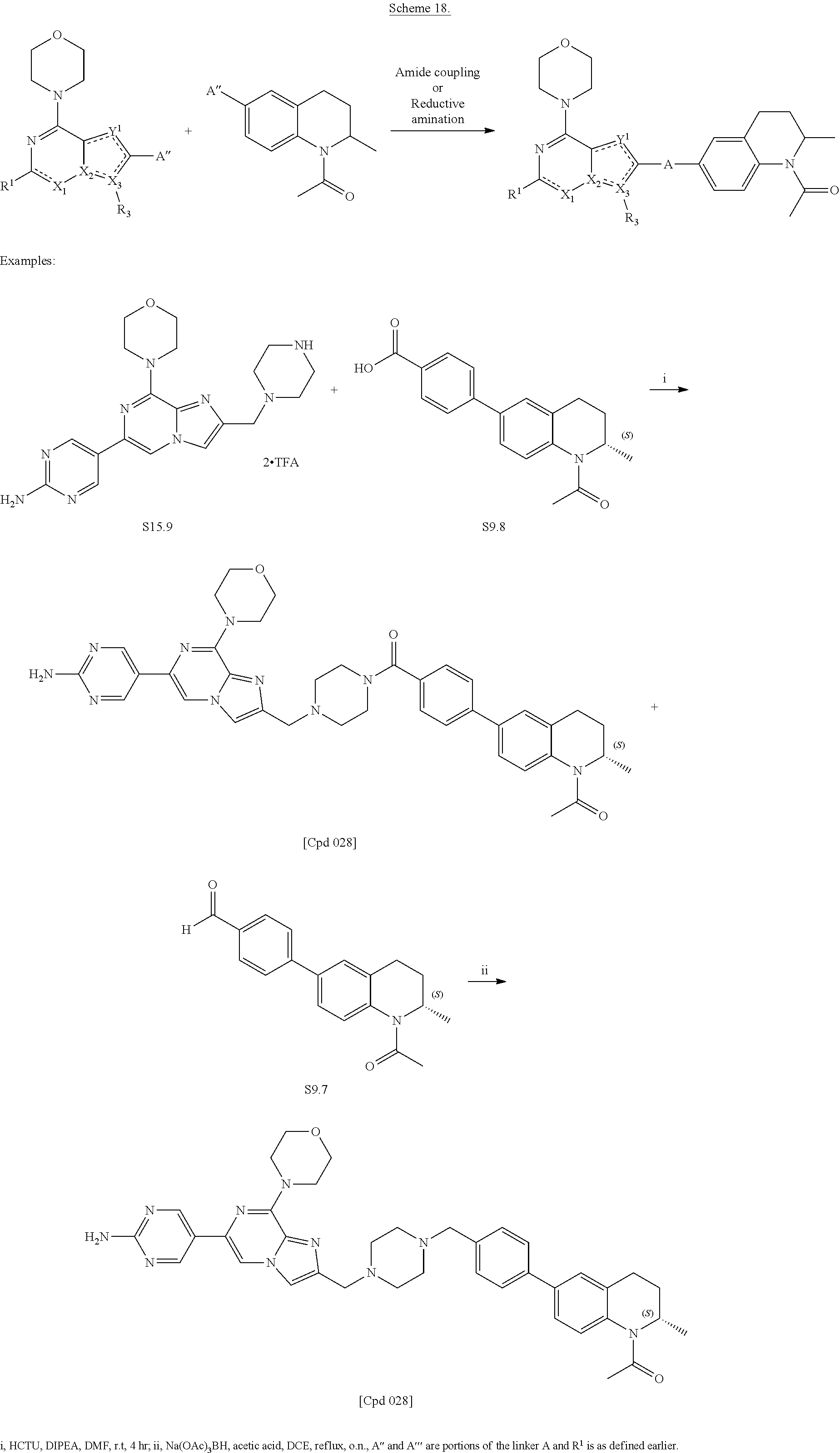

Compounds Cpd 014, 015, 028, and 029 were synthesised according to Scheme 18.

Scheme 19 details coupling of the left-hand side of compounds of Formula (I) with the right-hand side of the compounds of Formula (I), via the linker:

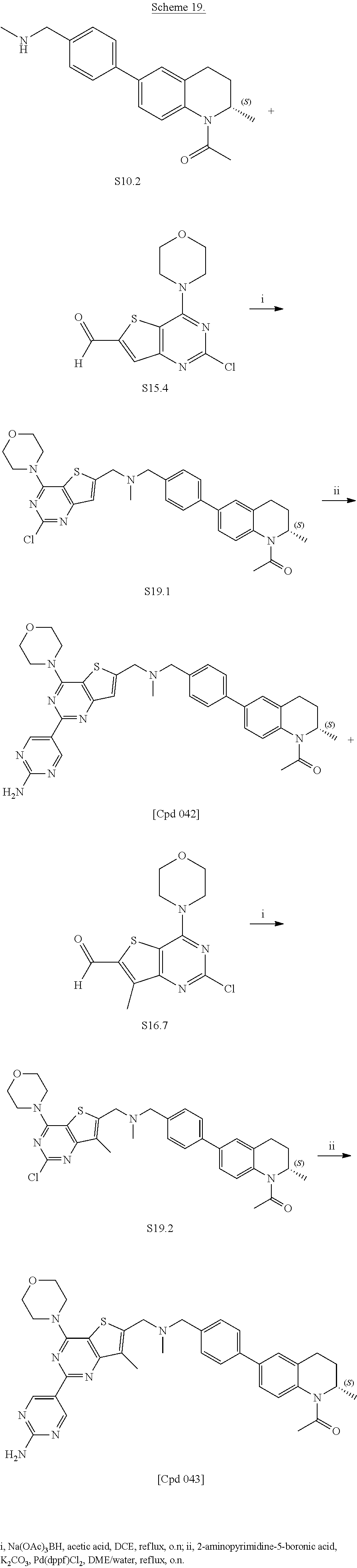

Compounds Cpd 042 and 043 were synthesised according to Scheme 19.

Scheme 20 details coupling of the left-hand side of compounds of Formula (I) with the right-hand side of the compounds of Formula (I), via the linker:

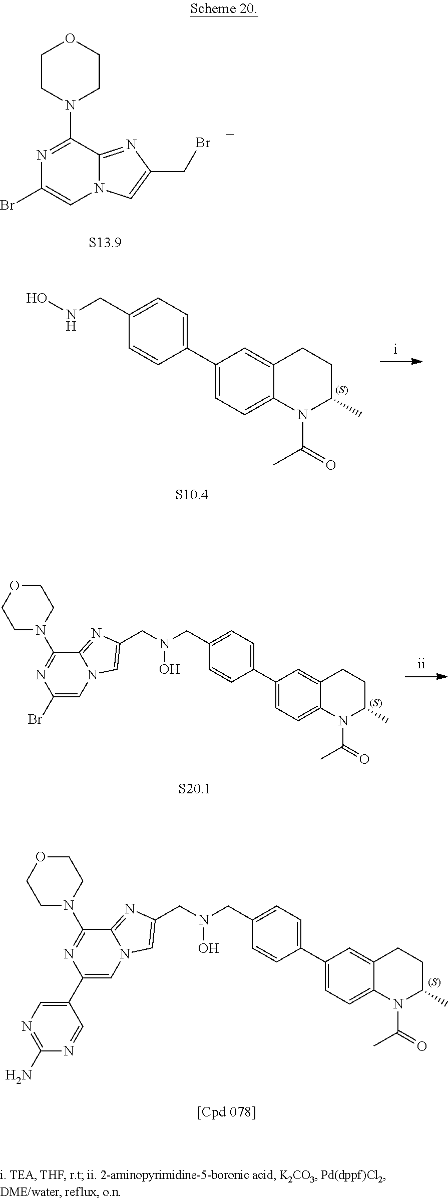

Compound Cpd 078 will be synthesised according to Scheme 20.

Scheme 21 details coupling of the left-hand side of compounds of Formula (I) with the right-hand side of the compounds of Formula (I), via the linker, and further functionalisation of the linker:

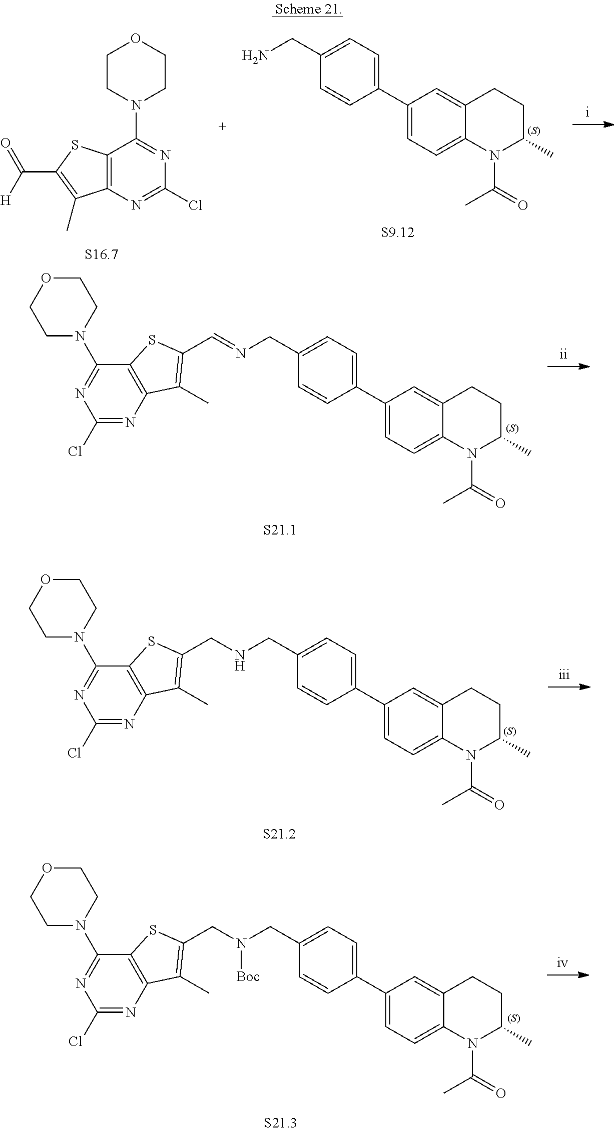

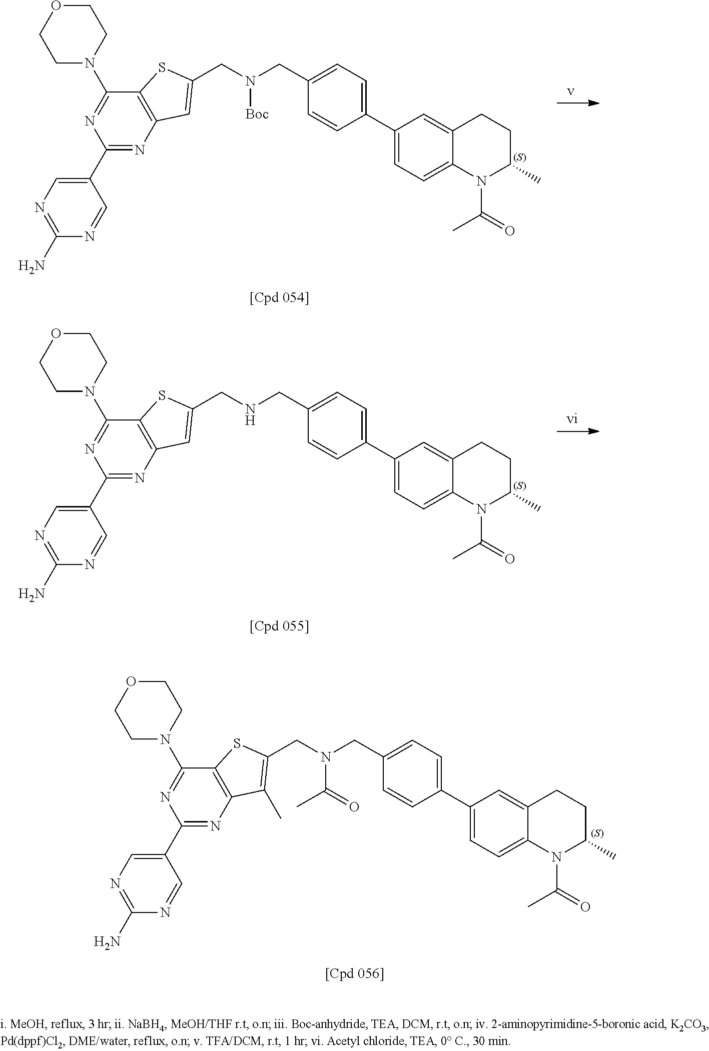

Compounds Cpd 054, 055, and 056 were synthesised according to Scheme 21.

### Preparation of Compound 035

Scheme 22 details coupling of the left-hand side of compounds of Formula (I) with the right-hand side of the compounds of Formula (I), via the linker:

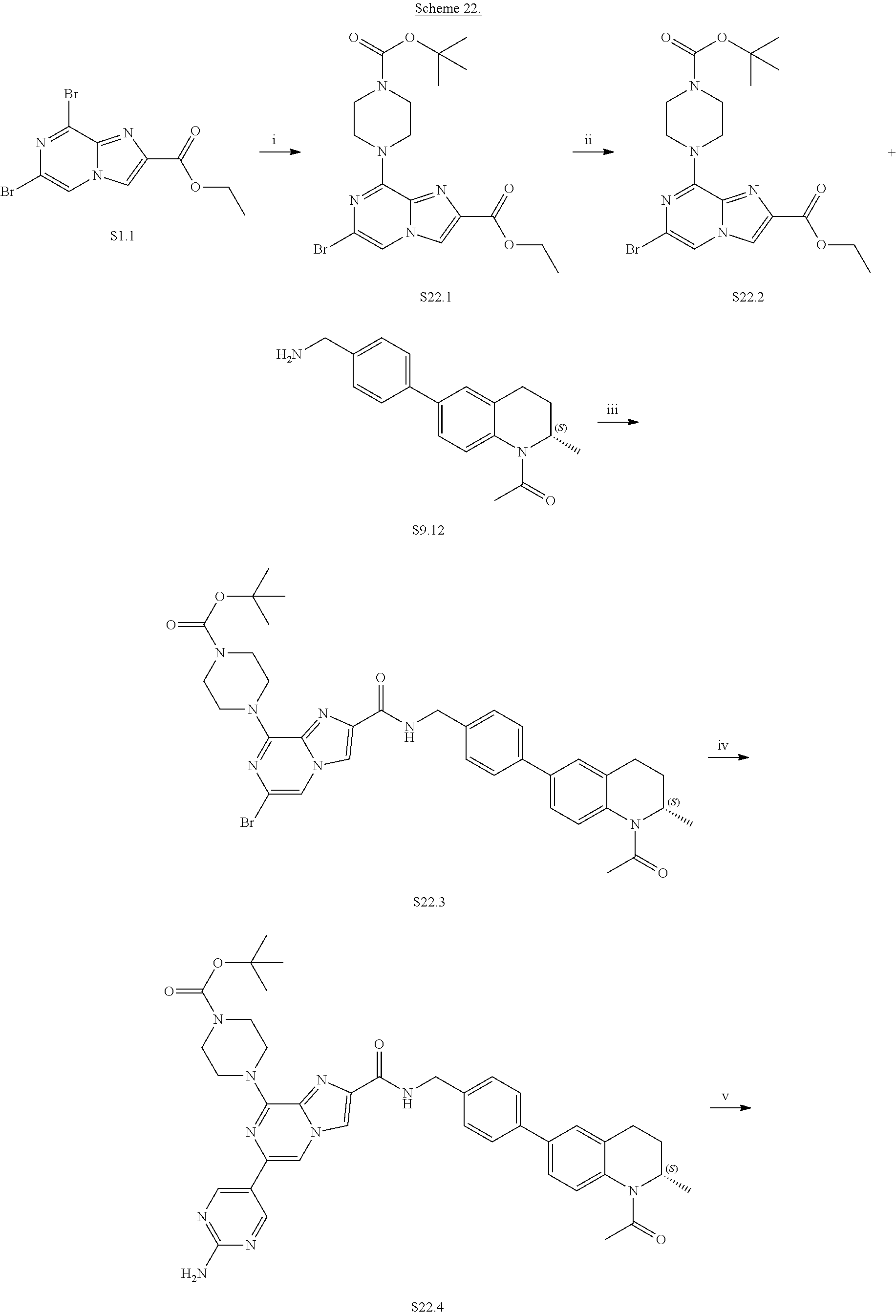

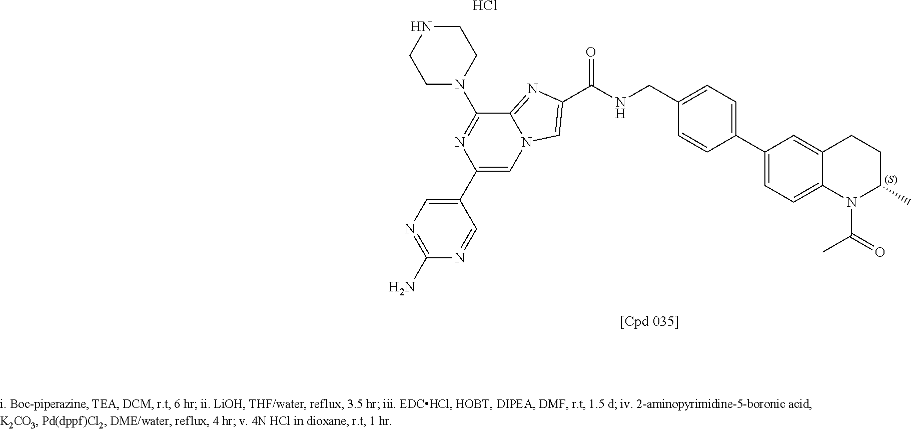

Compound Cpd 035 was synthesised according to Scheme 22.

### Preparation of Compound 064

Scheme 23 details coupling of the left-hand side of compounds of Formula (I) with the right-hand side of the compounds of Formula (I), via the linker:

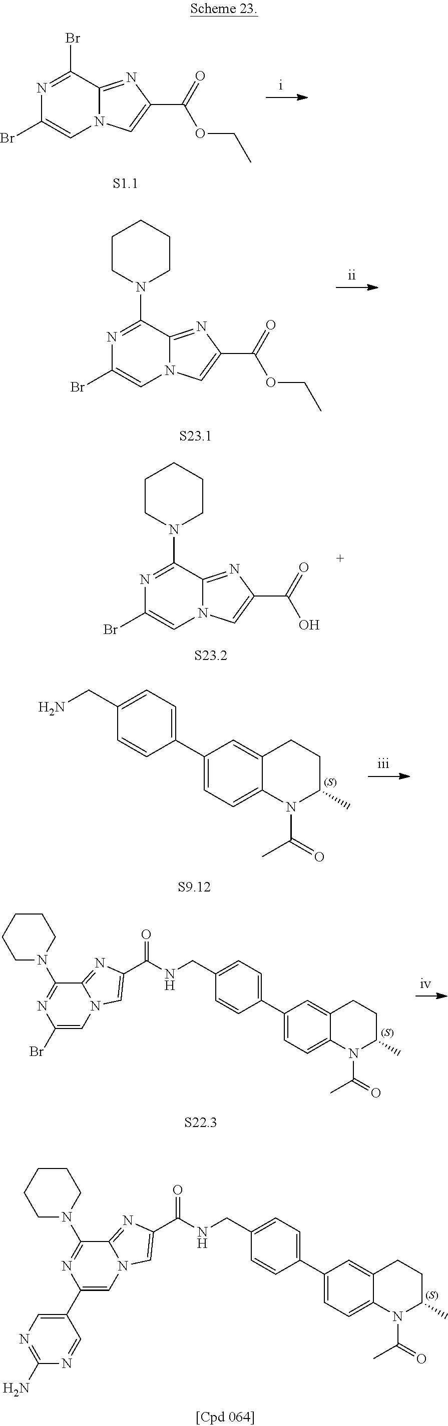

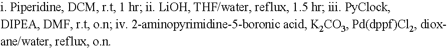

Compound Cpd 064 was synthesised according to Scheme 23.

### Preparation of Compound 065

Scheme 24 details coupling of the left-hand side of compounds of Formula (I) with the right-hand side of the compounds of Formula (I), via the linker:

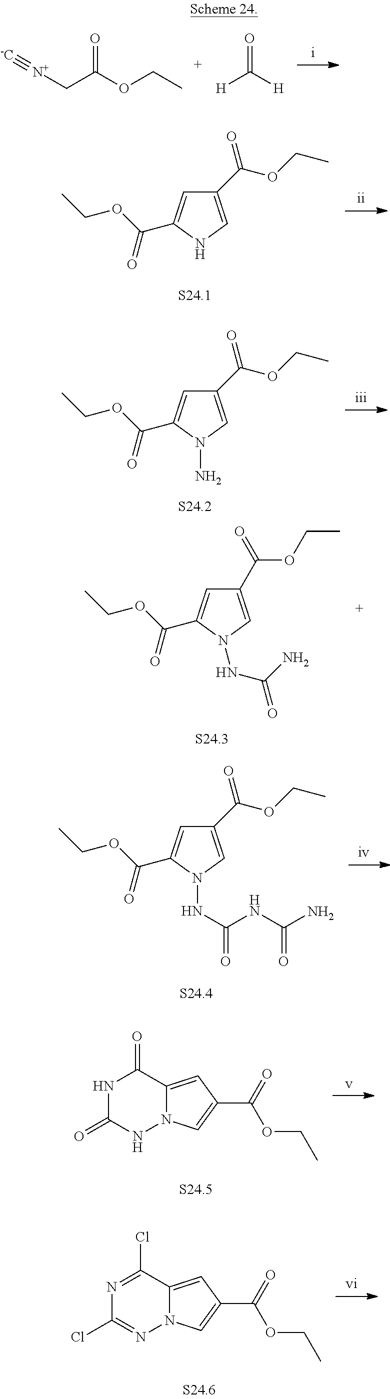

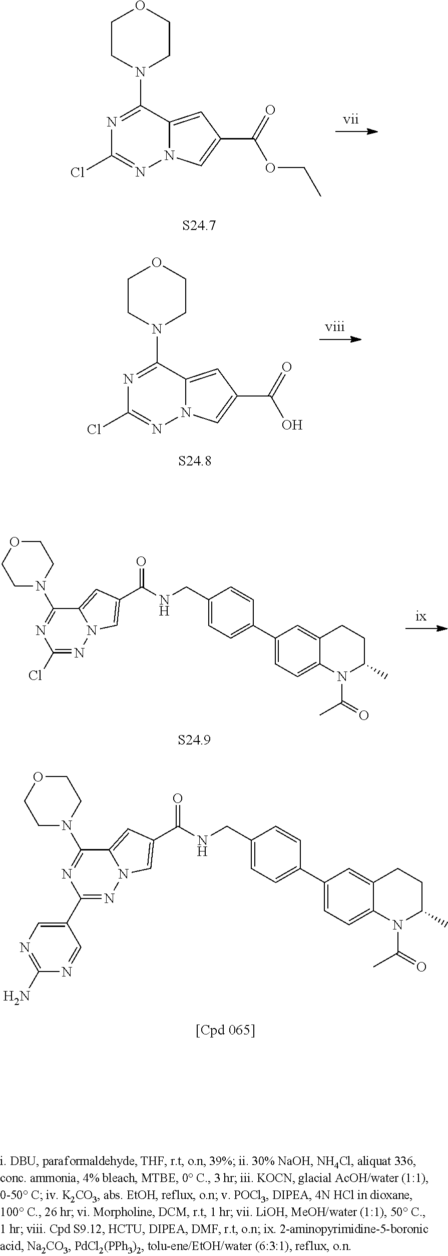 [SMILES: CCOC(=O)c1cc2c(N3CCOCC3)nc(Cl)nn2c1 ; O=C(O)c1cc2c(N3CCOCC3)nc(Cl)nn2c1 ; CC(=O)N1c2ccc(-c3ccc(CNC(=O)c4cc5c(N6CCOCC6)nc(Cl)nn5c4)cc3)cc2CC[C@@H]1C ; CC(=O)N1c2ccc(-c3ccc(CNC(=O)c4cc5c(N6CCOCC6)nc(-c6cnc(N)nc6)nn5c4)cc3)cc2CC[C@@H]1C]

Compound Cpd 065 was synthesised according to Scheme 24.

### Preparation of Compound 066

Scheme 25 details coupling of the left-hand side of compounds of Formula (I) with the right-hand side of the compounds of Formula (I), via the linker:

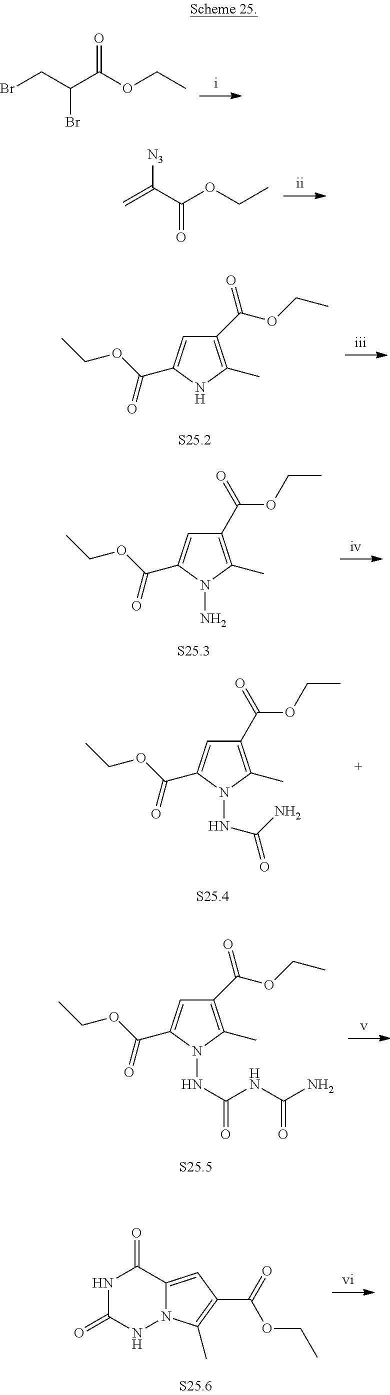

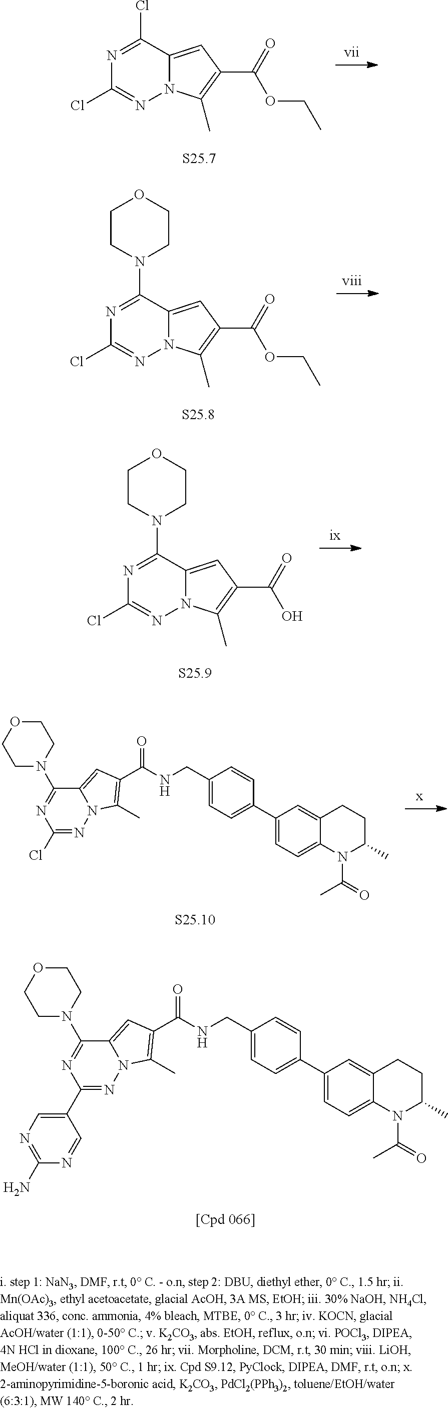 [SMILES: CCOC(=O)c1cc2c(Cl)nc(Cl)nn2c1C ; CCOC(=O)c1cc2c(N3CCOCC3)nc(Cl)nn2c1C ; Cc1c(C(=O)O)cc2c(N3CCOCC3)nc(Cl)nn12 ; CC(=O)N1c2ccc(-c3ccc(CNC(=O)c4cc5c(N6CCOCC6)nc(Cl)nn5c4C)cc3)cc2CC[C@@H]1C ; CC(=O)N1c2ccc(-c3ccc(CNC(=O)c4cc5c(N6CCOCC6)nc(-c6cnc(N)nc6)nn5c4C)cc3)cc2CC[C@@H]1C]

Compound Cpd 066 was synthesised according to Scheme 25.

### Preparation of Compounds 067 and 068

Scheme 26 details coupling of the left-hand side of compounds of Formula (I) with the right-hand side of the compounds of Formula (I), via the linker:

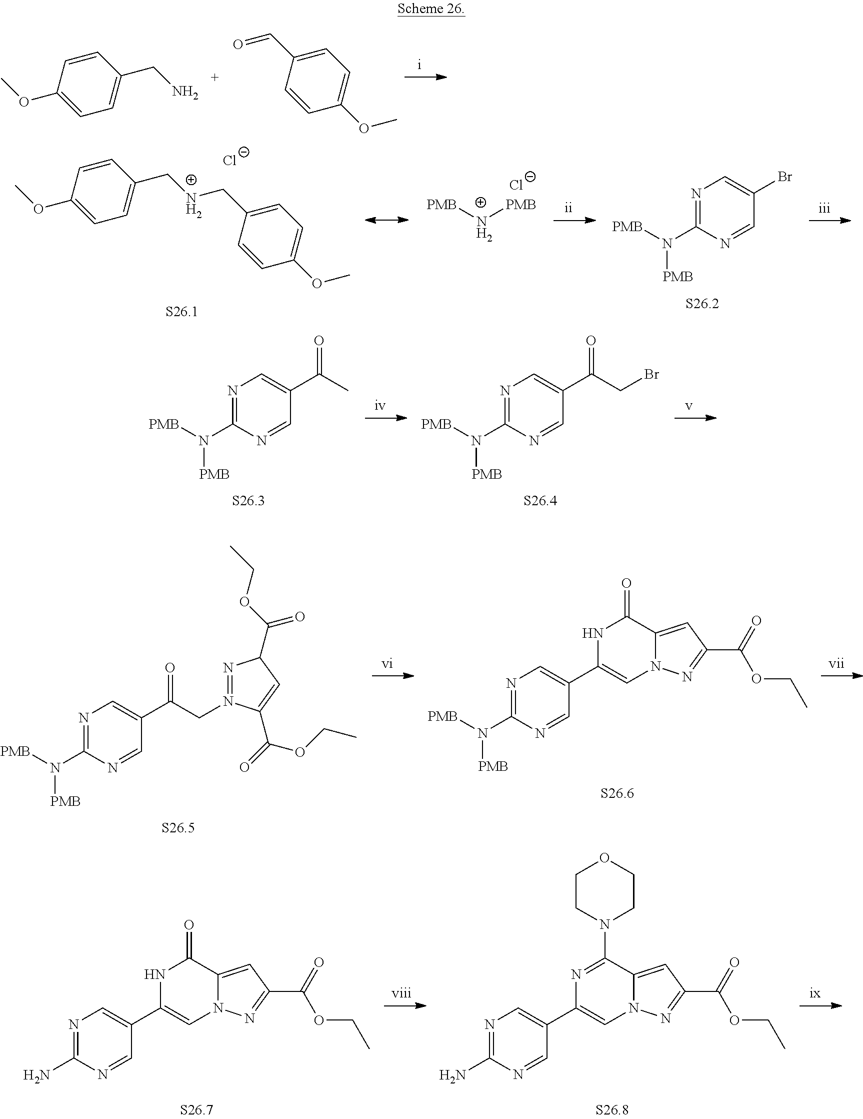

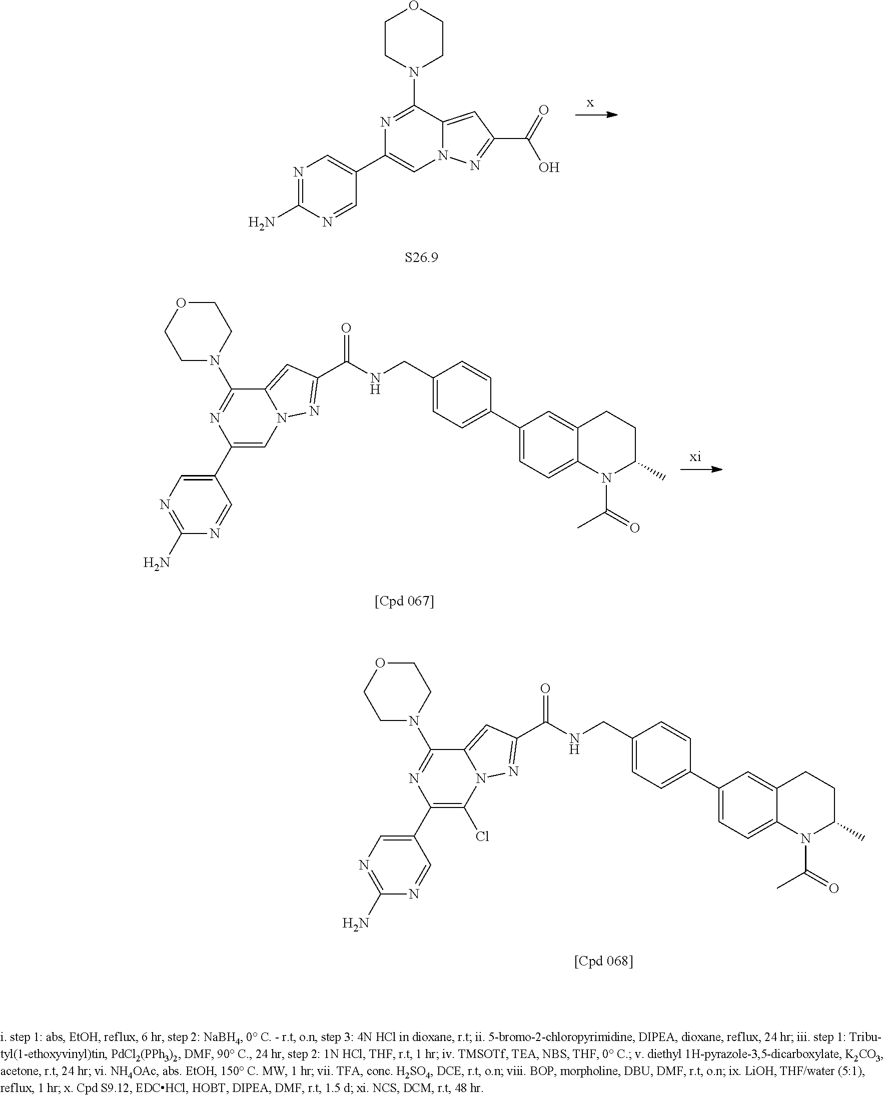 [SMILES: Nc1ncc(-c2cn3nc(C(=O)O)cc3c(N3CCOCC3)n2)cn1 ; CC(=O)N1c2ccc(-c3ccc(CNC(=O)c4cc5c(N6CCOCC6)nc(-c6cnc(N)nc6)cn5n4)cc3)cc2CC[C@@H]1C ; CC(=O)N1c2ccc(-c3ccc(CNC(=O)c4cc5c(N6CCOCC6)nc(-c6cnc(N)nc6)c(Cl)n5n4)cc3)cc2CC[C@@H]1C]

Compounds Cpd 067 and 068 were synthesised according to Scheme 26.

### Preparation of Compounds 069

Scheme 27 details coupling of the left-hand side of compounds of Formula (I) with the right-hand side of the compounds of Formula (I), via the linker:

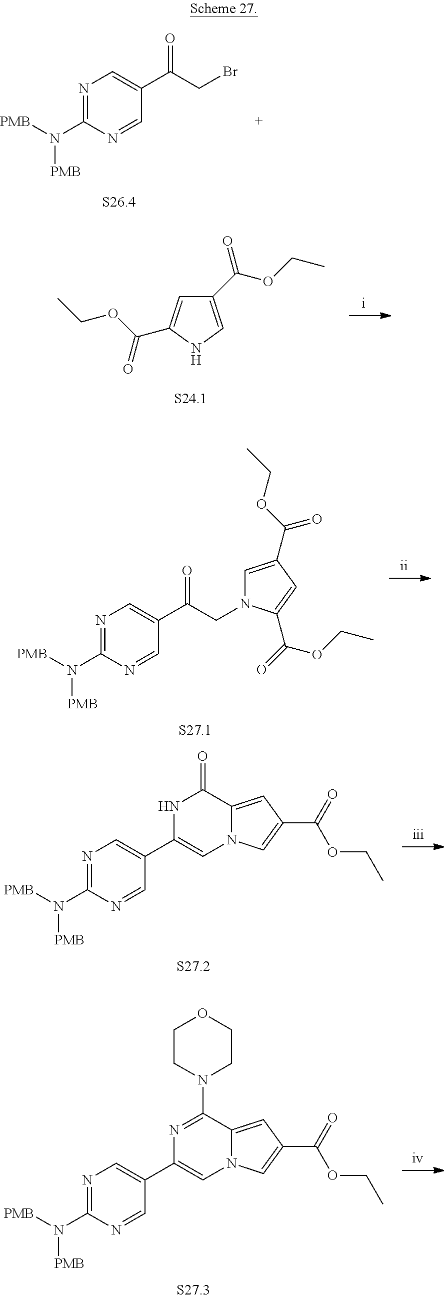

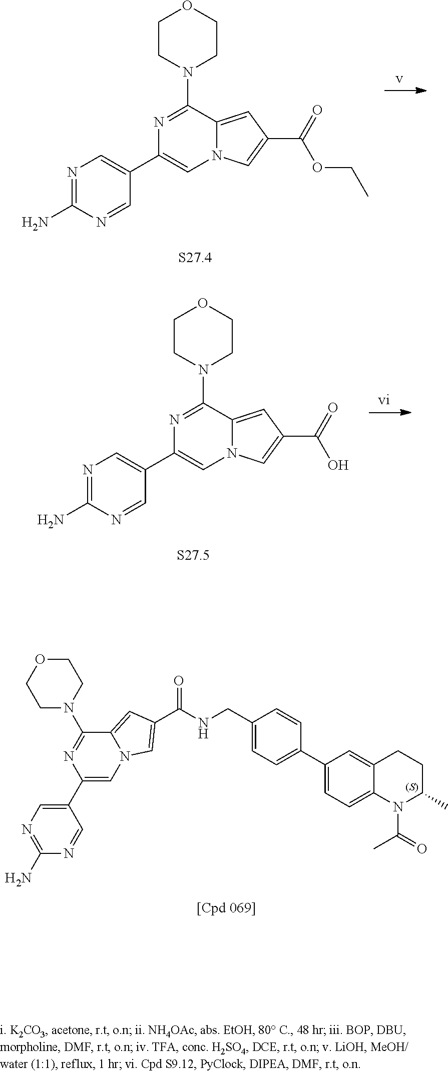

Compound Cpd 069 was synthesised according to Scheme 27.

### Preparation of Compound 070

Scheme 28 details coupling of the left-hand side of compounds of Formula (I) with the right-hand side of the compounds of Formula (I), via the linker:

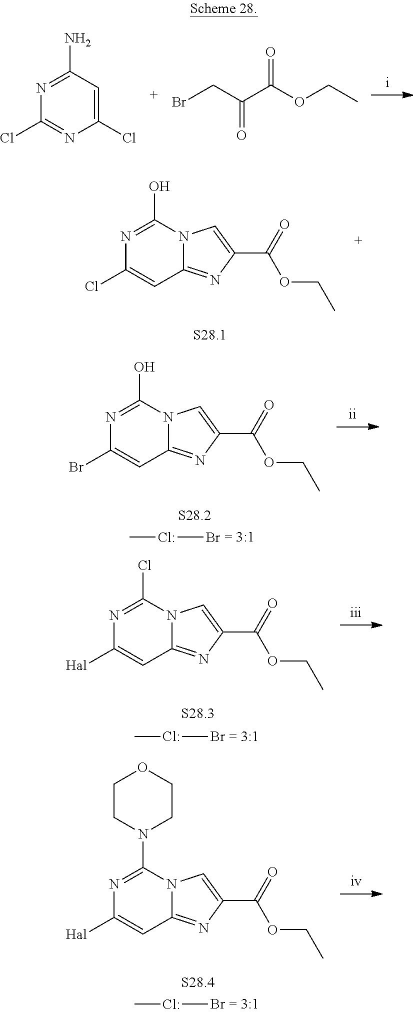

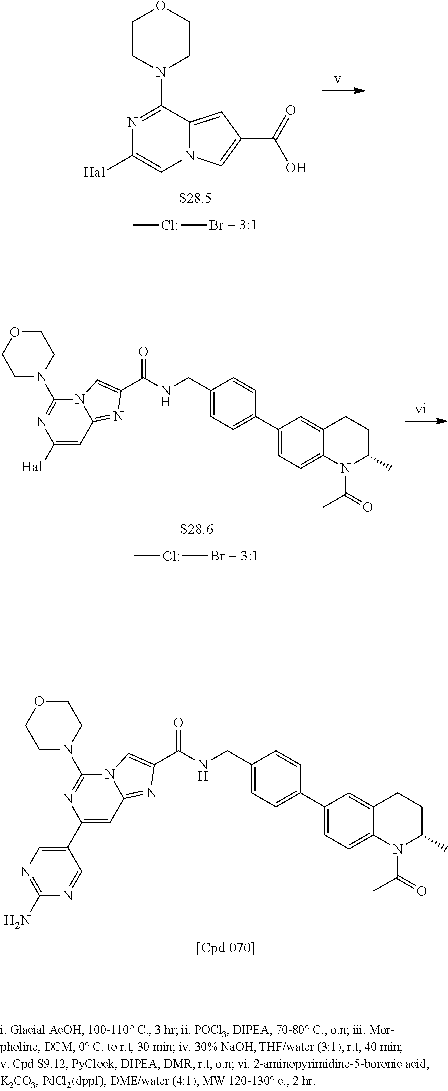

Compound Cpd 070 was synthesised according to Scheme 28.

### Preparation of Compound 071

Scheme 29 details coupling of the left-hand side of compounds of Formula (I) with the right-hand side of the compounds of Formula (I), via the linker:

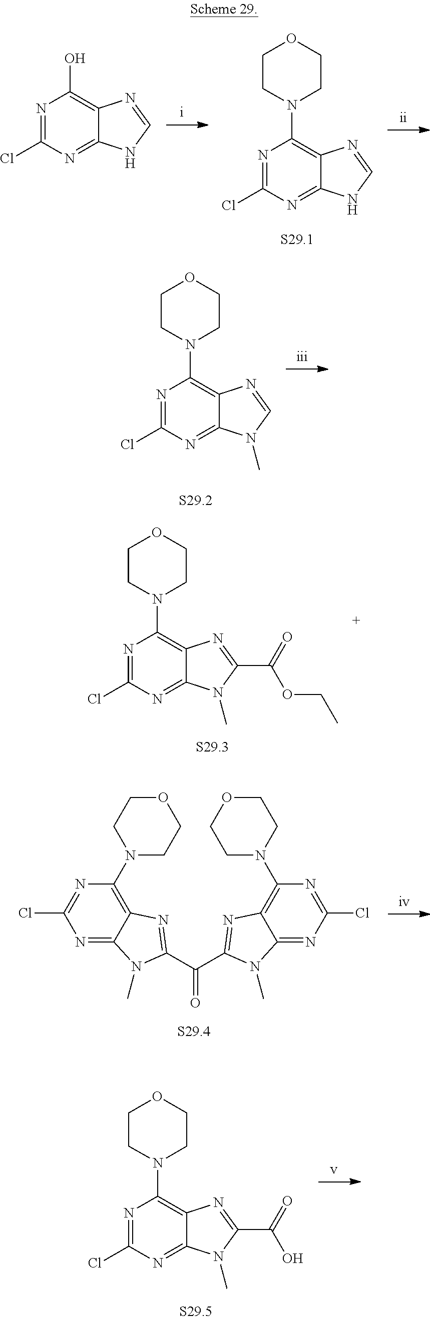 [SMILES: Oc1nc(Cl)nc2[nH]cnc12 ; Clc1nc(N2CCOCC2)c2nc[nH]c2n1 ; Cn1cnc2c(N3CCOCC3)nc(Cl)nc21 ; CCOC(=O)c1nc2c(N3CCOCC3)nc(Cl)nc2n1C ; Cn1c(C(=O)c2nc3c(N4CCOCC4)nc(Cl)nc3n2C)nc2c(N3CCOCC3)nc(Cl)nc21 ; Cn1c(C(=O)O)nc2c(N3CCOCC3)nc(Cl)nc21]

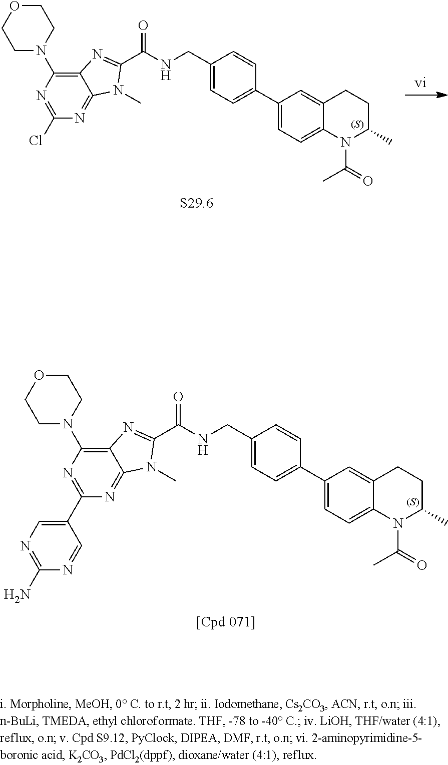

Compound Cpd 071 was synthesised according to Scheme 29.

### Preparation of Compound 072

Scheme 30 details coupling of the left-hand side of compounds of Formula (I) with the right-hand side of the compounds of Formula (I), via the linker:

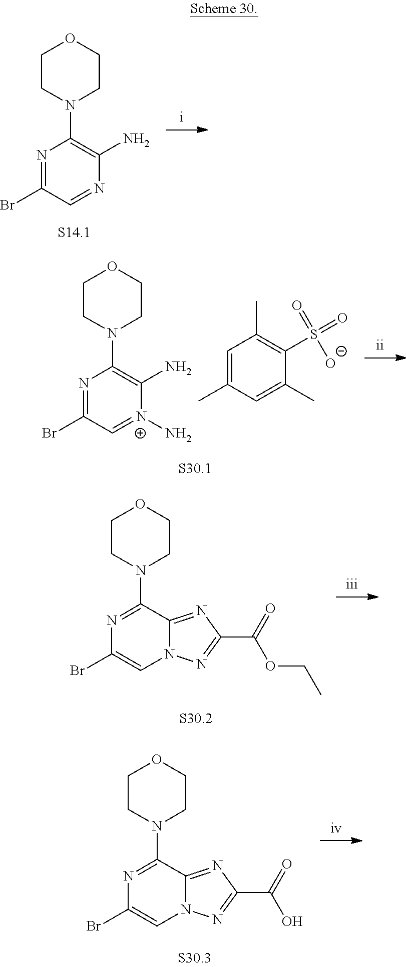 [SMILES: Nc1ncc(Br)nc1N1CCOCC1 ; Nc1c(N2CCOCC2)nc(Br)c[n+]1N ; Cc1cc(C)c(S(=O)(=O)[O-])c(C)c1 ; CCOC(=O)c1nc2c(N3CCOCC3)nc(Br)cn2n1 ; O=C(O)c1nc2c(N3CCOCC3)nc(Br)cn2n1]

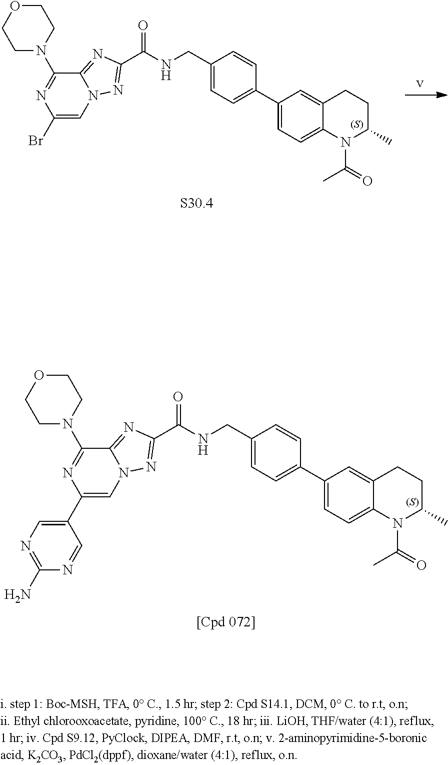

Compound Cpd 072 was synthesised according to Scheme 30.

### Preparation of Compound 073

Scheme 31 details coupling of the left-hand side of compounds of Formula (I) with the right-hand side of the compounds of Formula (I), via the linker:

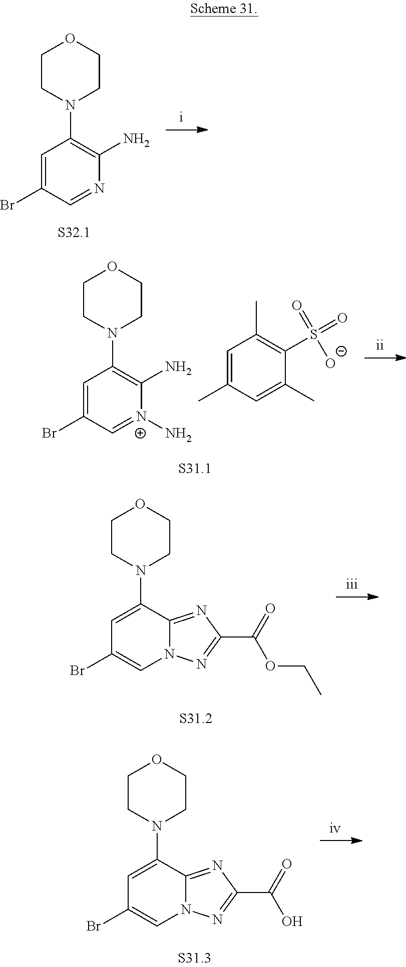 [SMILES: Nc1ncc(Br)cc1N1CCOCC1 ; Nc1c(N2CCOCC2)cc(Br)c[n+]1N ; Cc1cc(C)c(S(=O)(=O)[O-])c(C)c1 ; CCOC(=O)c1nc2c(N3CCOCC3)cc(Br)cn2n1 ; O=C(O)c1nc2c(N3CCOCC3)cc(Br)cn2n1]

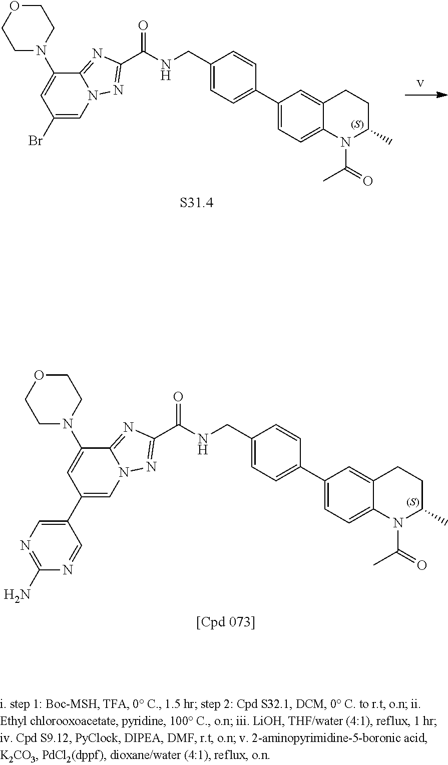

Compound Cpd 073 was synthesised according to Scheme 31.

### Preparation of Compound 074

Scheme 32 details coupling of the left-hand side of compounds of Formula (I) with the right-hand side of the compounds of Formula (I), via the linker:

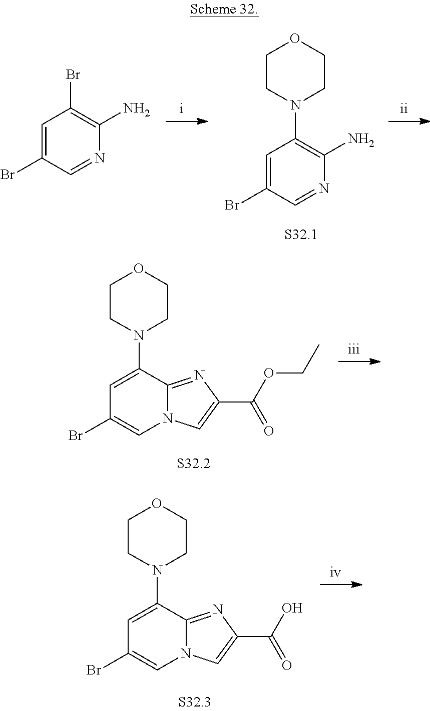 [SMILES: Nc1ncc(Br)cc1Br ; Nc1ncc(Br)cc1N1CCOCC1 ; CCOC(=O)c1cn2cc(Br)cc(N3CCOCC3)c2n1 ; O=C(O)c1cn2cc(Br)cc(N3CCOCC3)c2n1]

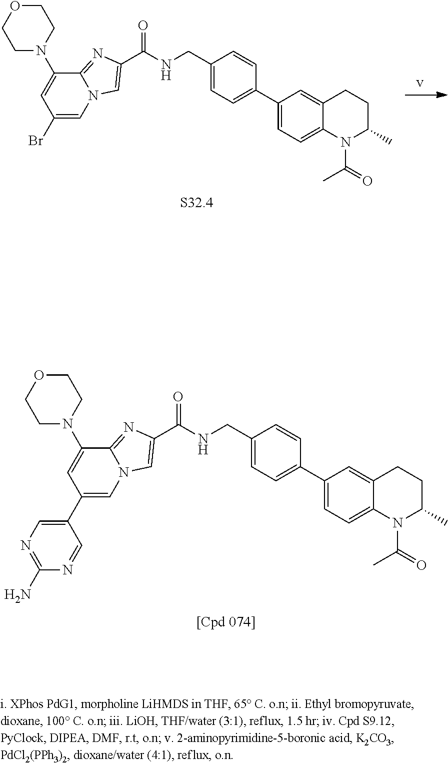 [SMILES: CC(=O)N1c2ccc(-c3ccc(CNC(=O)c4cn5cc(Br)cc(N6CCOCC6)c5n4)cc3)cc2CC[C@@H]1C ; CC(=O)N1c2ccc(-c3ccc(CNC(=O)c4cn5cc(-c6cnc(N)nc6)cc(N6CCOCC6)c5n4)cc3)cc2CC[C@@H]1C]

Compound Cpd 074 was synthesised according to Scheme 32.

### Preparation of Compound 075

Scheme 33 details coupling of the left-hand side of compounds of Formula (I) with the right-hand side of the compounds of Formula (I), via the linker:

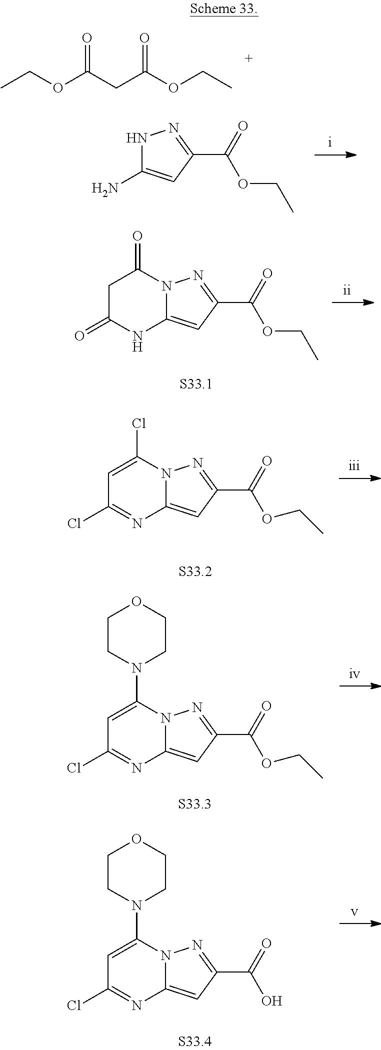

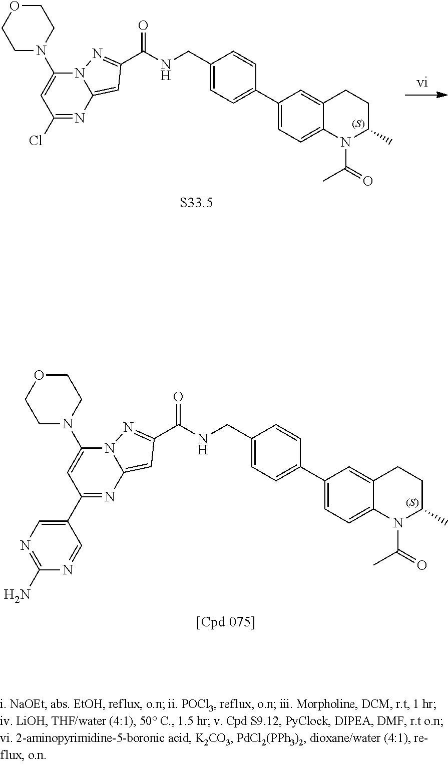

Compound Cpd 075 was synthesised according to Scheme 33.

### Preparation of Compounds 076 and 077

Scheme 34 details coupling of the left-hand side of compounds of Formula (I) with the right-hand side of the compounds of Formula (I), via the linker:

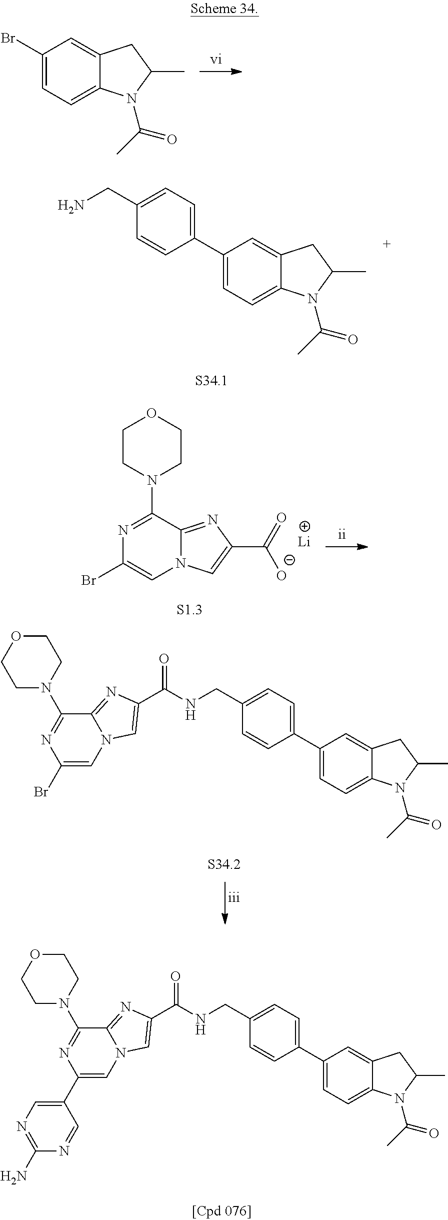

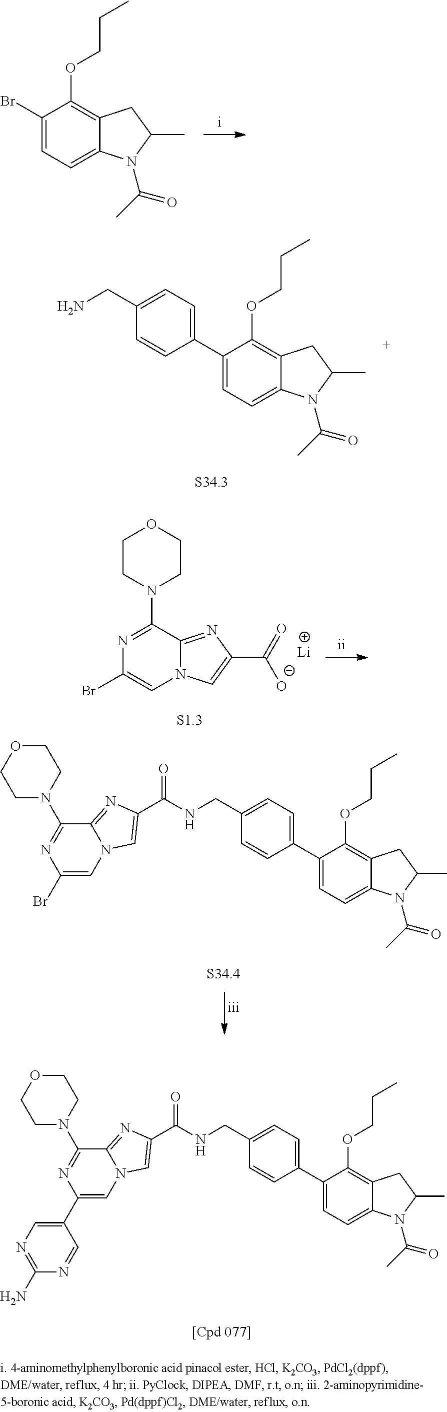 [SMILES: CCCOc1c(Br)ccc2c1CC(C)N2C(C)=O ; CCCOc1c(-c2ccc(CN)cc2)ccc2c1CC(C)N2C(C)=O ; O=C([O-])c1cn2cc(Br)nc(N3CCOCC3)c2n1 ; [Li+] ; CCCOc1c(-c2ccc(CNC(=O)c3cn4cc(Br)nc(N5CCOCC5)c4n3)cc2)ccc2c1CC(C)N2C(C)=O ; CCCOc1c(-c2ccc(CNC(=O)c3cn4cc(-c5cnc(N)nc5)nc(N5CCOCC5)c4n3)cc2)ccc2c1CC(C)N2C(C)=O]

Compounds Cpd 076 and 077 were synthesised according to Scheme 34.

### General Chemistry Methods

All solvents were purchased from Merck and all reagents were purchased from Sigma-Aldrich. All MSDSs were consulted before conducting each experiment.

¹H and ¹³C Nuclear Magnetic Resonance spectra were conducted on a Bruker Advance III Nanobay 400 MHz spectrometer coupled to the BACS 60 automatic sample changer and obtained at 400.13 MHz and 100.62 MHz respectively. All spectra were processed using Mes-tReNova 6.0 software. The chemical shifts of ¹H and ¹³C spectra are reported in parts per million (ppm). The chemical shifts of all ¹H NMR were measured relative to the expected solvent peaks of the respective NMR solvents; CDCl₃, 7.26; MeOD, 3.31; DMSO-D₆, 2.50. When a mixture of CDCl₃ and MeOD was used as the solvent, the residual solvent peak for CDCl₃ was used as the internal reference. The data for all spectra are reported in the following format: chemical shift (multiplicity, coupling constant, integration, assignment). Multiplicity is defined as: s=singlet, d=doublet, t=triplet, q=quartet, sd=singlet of doublets, dd=doublet of doublets, dt=doublet of triplets, tt=triplet of triplets and m=multiplet A broad resonance is denoted by the abbreviation br and the apparent sliptting is denoted as the abbreviation app. The assignment n/n′ is used to denote non-magnetically equivalent protons. Coupling constants are applied as J in Hertz (Hz). For ¹³C NMR, only chemical shifts are reported.

All HRMS analyses were done on an Agilent 6224 TOF LC/MS Mass Spectrometer coupled to an Agilent 1290 Infinity (Agilent, Palo Alto, CA). All data were acquired and reference mass corrected via a dual-spray electrospray ionization (ESI) source. Each scan or data point on the Total Ion Chromatogram (TIC) is an average of 13,700 transients, producing a spectrum every second. Mass spectra were created by averaging the scans across each peak and background subtracted against the first 10 seconds of the TIC. Acquisition was performed using the Agilent Mass Hunter Fata Acquisition software version B.05.00 Build 5.0.5042.2 and analysis was performed using Mass Hunter Qualitative Analysis version B.05.00 Build 5.0.519.13.

All LCMS analyses were carried out on an Agilent 6100 Series Single Quad LC/MS coupled with an Agilent 1200 Series HPLC, 1260 Infinity G4212A 1290 DAD detector. The liquid chromatography conditions were: Reverse phase HPLC analysis fitted with a Luna C-8 (2) 5 μl×4.6 mm 100 Å at a temperature of 30° C. The sample injection volume was 1 or 2 μl, which was run in 0.1% formic acid in acetonitrile at a gradient of 0-100% over 5 or 10 min. Detection methods were either 254 nm or 214 nm. The low resolution mass spectrum was per formed using electron spray ionization. Conditions: Quadrupole ion source with Multimode-ES, The drying gas temperature was 300° C. and the vaporizer temperature was 200° C. The capillary voltage in positive mode was 2000V, while in negative mode, the capillary voltage was 4000V. The scan range was 100-1000 m/z with a step size of 0.1 second over 10 min.

Purification on reverse-phase HPLC was done on a Waters Delta Prep 2000 Prep HPLC system that was fitted with a Waters Delta Prep 2000 Pump and Controller. Samples were in-jected into a Waters Prep Rack with Manual Injector, which were run through a Luna C8(2) 10 uL 50×21.20 mm 100 Å and Waters 486 Tunable Absorbance Detector. The conditions were: Solvent A (0.1% TFA in H₂O) and Solvent B (0.1% TFA in acetonitrile), at a gradient of 0-100% B over 60 min at either 10 or 15 mL/min.

### Synthesis and Characterization

### Ethyl 6,8-dibromoimidazo[1,2-a]pyrazine-2-carboxylate (S1.1)

To a mixture of 2-amino-3,5-dibromopyrazine (3.0 g, 11.86 mmol) in anhydrous DME (12 mL) was added ethyl bromopyruvate (2 mL˜90% pure, 14.34 mmol) at r.t. The reaction mixture was heated at 80° C. for 24 hr. The mixture was then diluted with DCM, washed with sat. NaHCO₃ solution (×3), dried over MgSO₄ and the solvent concentrated in vacuo. The residue was then triturated with cold diethyl ether and the solid was collected via filtration to give the title compound as a fawn color powder (3.26 g, 79%). This material was used in the next step without further purification. ¹H NMR (400 MHz, CDCl₃) δ 8.29 (s, 1H), 8.25 (s, 1H), 4.49 (q, J=7.1 Hz, 2H), 1.44 (t, J=7.1 Hz, 3H). LCMS: (0-100% Acetonitrile over 5 min, 0.1% formic acid throughout) Rf=3.20 min, (ESI) m/z: 349.7 (M+H, 100%).

### Ethyl 6-bromo-8-morpholinoimidazo[1,2-a]pyrazine-2-carboxylate (S1.2)

To a solution of ethyl 6,8-dibromoimidazo[1,2-a]pyrazine-2-carboxylate (S1.1) (3.26 g, 9.34 mmol) in DCM (40 mL) was added morpholine (3.26 mL, 37.80 mmol) dropwise. The resulting suspension was stirred at r.t for 3 hr. The mixture was then diluted with DCM, washed with sat. NaHCO₃ solution (×3), dried over MgSO₄ and concentrated in vacuo. The residue was triturated with cold diethyl ether and the solid was collected via filtration to give the title compound as a pale yellow solid (2.94 g, 89%). This material was used in the next step without further purification. ¹H NMR (400 MHz, CDCl₃) δ 7.97 (s, 1H), 7.58 (s, 1H), 4.39 (m, 6H), 3.85 (t, J=4.9 Hz, 4H), 1.41 (t, J=7.1 Hz, 3H). LCMS: (0-100% Acetonitrile over 10 min, 0.1% formic acid throughout) Rf=5.86 min, (ESI) m/z: 355.0 (M+H, 100%).

### Lithium (I) 6-bromo-8-morpholinoimidazo[1,2-a]pyrazine-2-carboxylate (S1.3)

To a stirred solution of ethyl 6-bromo-8-morpholinoimidazo[1,2-a]pyrazine-2-carboxylate (S1.2) (1.5 g, 4.22 mmol) in a mixture of THE and water (4:1, 30 mL) was added LiOH monohydrate (266 mg, 6.34 mmol). The reaction mixture was then heated to reflux for 3 hr. The organic solvent was concentrated in vacuo. To the residual aqueous layer was then added acetone (50 mL) and the resulting precipitate was collected via filtration and dried under vacuum to give the title compound as an off-white to white powder (1.31 g, 93%). ¹H NMR (400 MHz, MeOD) δ 8.00 (s, 1H), 7.91 (s, 1H), 4.33 (t, J=4.8 Hz, 4H), 3.81 (t, J=4.9 Hz, 4H). LCMS: (0-100% Acetonitrile over 10 min, 0.1% formic acid throughout) Rf=3.09 min, (ESI) m/z: 326.8 (M+H, 100%).

### Lithium 6-(2-aminopyrimidin-5-yl)-8-morpholinoimidazo[1,2-a]pyrazine-2-carboxylate (S1.4)

A suspension of lithium (I) 6-bromo-8-morpholinoimidazo[1,2-a]pyrazine-2-carboxylate (S1.3) (386 mg, 1.16 mmol), K₂CO₃ (479 mg, 3.47 mmol), 2-aminopyrimidine-5-boronic acid (195 mg, 1.40 mmol) in a mixture of DME/water (4:1, 5 mL) was degassed with N₂ for 15 min. Pd(dppf)Cl₂ (68 mg, 0.12 mmol) was then added and the mixture was further degassed for another 15 min. The reaction vessel was then evacuated and backfilled with N₂ three times. The reaction mixture was heated to reflux for 20 hr. The reaction mixture was then diluted with water, filtered through Celite and the filter rinsed with water. The filtrate was freeze-dried, the solid was then taken up into MeOH and filtered. The filtrate was concentrated in vacuo to give the crude title compound as the lithium salt contaminated by the potassium salt. (957 mg, ˜40% pure) (nb. purity taken into account in subsequent steps). ¹H NMR (400 MHz, MeOD) δ 8.83 (s, 2H), 8.26 (s, 1H), 8.08 (s, 1H), 4.36 (t, J=4.9 Hz, 4H), 3.87 (t, J=4.8 Hz, 4H), 1.89 (s, 2H). LCMS: (0-100% Acetonitrile over 10 min, 0.1% formic acid throughout) Rf=2.806 min, (ESI) m/z: 341.9 (M+H, 100%).

### tert-Butyl 4-(6-(2-aminopyrimidin-5-yl)-8-morpholinoimidazo[1,2-a]pyrazine-2-carbonyl)piperazine-1-carboxylate (S1.5)

To a mixture of lithium 6-(2-aminopyrimidin-5-yl)-8-morpholinoimidazo[1,2-a]pyrazine-2-carboxylate (S1.4) (100 mg, 0.29 mmol) in DMF (3 mL) was added HCTU (360 mg, 0.87 mmol), DIPEA (225 mg, 1.74 mmol) and 1-Boc piperazine (67 mg, 0.36 mmol). This mixture was then stirred at room temperature for 2 hr. The reaction mixture was then added into distilled water (50 mL). The resulting precipitate was collected and triturated with diethyl ether to give the tile product as a pale yellow powder (67 mg, 45%). ¹H NMR (400 MHz, CDCl₃) δ 8.83 (s, 2H), 8.07 (s, 1H), 7.82 (s, 1H), 5.47 (s, 2H), 4.36 (br s, 4H), 4.32-4.12 (m, 2H), 3.89 (t, J=4.8 Hz, 4H), 3.79 (br s, 2H), 3.55 (br s, 4H), 1.50 (s, 9H). LCMS: (0-100% Acetonitrile over 5 min, 0.1% formic acid throughout) Rf=3.176 min, (ESI) m/z: 510.0 (M+H, 100%).

### 1-(2-Methyl-3,4-dihydroquinolin-1(2H)-yl)ethan-1-one (S2.1)

To a stirred solution of 2-methyl-1,2,3,4-tetrahydroquinoline (1.033 g, 7.02 mmol) in CHCl₃ (50 mL) was added DIPEA (2.710 g. 21.01 mmol) and added dropwise a solution of acetic anhydride (1.454 g, 14.24 mmol). The solution was heated to reflux and stirred for 5 hr. This solution was cooled to room temperature and then washed successively with water (50 mL), sat. NaHCO₃ solution (2×50 mL), brine (50 mL) and dried over MgSO₄. The organic solvent was then removed in vacuo to provide the title compound as a pale brown oil which crystalized on standing. (1.114 g, 86%). ¹H NMR (400 MHz, CDCl₃) δ 7.25-7.04 (m, 4H), 4.86 (br s, 1H), 2.69-2.45 (m, 2H), 2.37-2.26 (m, 1H), 1.40-1.25 (m, 1H), 2.16 (s, 3H), 1.14 (d, J=4.0 Hz, 3H). LCMS: (0-100% Acetonitrile over 5 min, 0.1% formic acid through out) Rf=3.212 min, (ESI) m/z: 190.1 (M+H, 100%).

### 1-(6-Bromo-2-methyl-3,4-dihydroquinolin-1(2H)-yl)ethan-1-one (S2.2)

A solution of 1-(2-methyl-3,4-dihydroquinolin-1(2H)-yl)ethan-1-one (S2.1) (1.35 g, 7.12 mmol) in DCM (20 mL) was cooled to 0° C. and added NBS (1.52 g, 8.51 mmol). The mixture was stirred at this temperature for 2 hr and then warmed to room temperature for 12 hr. The reaction mixture was diluted with DCM (30 mL), washed with 10% NaOH solution (2×50 mL) and brine. The organic phase was dried over MgSO₄ and then concentrated to give the title compound as a clear yellow oil (1.90 g, 99%). ¹H NMR (400 MHz, CDCl₃) δ 7.32 (m, 2H), 7.03 (br s, 1H), 4.78 (br s, 1H), 2.62-2.46 (m, 2H), 2.36-2.28 (m, 1H), 2.13 (s, 3H), 1.35 (br s, 1H), 1.11 (d, J=6.5 Hz, 3H). LCMS: (0-100% Acetonitrile over 5 min, 0.1% formic acid throughout) Rf=3.395 min, (ESI) m/z: 267.9 (M+H, 100%).

### 4-(1-Acetyl-2-methyl-1,2,3,4-tetrahydroquinolin-6-yl)benzoic acid (S2.3)

To a sealed vial was added 1-(6-bromo-2-methyl-3,4-dihydroquinolin-1(2H)-yl)ethan-1-one (S2.2) (485 mg, 1.81 mmol), 4-Carboxyphenylboronic acid 0150 mg, 2.71 mmol) and K₂CO₃ (750 mg, 5.43 mmol) in a mixture of anhydrous DME and distilled water (4:1, 10 mL). A stream of nitrogen gas was bubbled through this suspension for 15 min. This suspension was then added PdCl₂(dppf) (95 mg, 0.13 mmol) and a stream of nitrogen gas was bubbled through this suspension for further 15 min. This reaction mixture was heated at 100° C. under nitrogen and stirred for 4 hr. To the reaction mixture was added distilled water (50 mL). The aqueous phase was washed with ethyl acetate (3×50 mL). The aqueous phase was then acidified to pH=1 by dropwise adding 1N HCl solution and extracted with DCM (3×50 mL). The organic extracts were combined, washed with brine and the solvent removed in vacuo. The resulting crude material was then purified by flash column chromatography (0-20% ethyl acetate in DCM+1% acetic acid) to give the title compound as a white powder (498 mg, 89%). ¹H NMR (400 MHz, CDCl₃) 8.18 (d, J=8.0 Hz, 2H), 7.79 (d, J=8.0 Hz, 2H), 7.50 (d, J=8.0 Hz, 1H), 7.46 (s, 1H), 7.28 (br s, 1H), 4.84 (br s, 1H), 2.74-2.59 (m, 2H), 2.40-2.37 (m, 1H), 2.24 (s, 3H), 1.68 (d, J=6.4 Hz, 3H), 1.47-1.35 (m, 1H). LCMS: (0-100% Acetonitrile over 5 min, 0.1% formic acid throughout) Rf=3.361 min, (ESI) m/z: 310.2 (M+H, 100%).

### 1-(6-(4-(Aminomethyl)phenyl)-2-methyl-3,4-dihydroquinolin-1(2H)-yl)ethan-1-one (S2.4)

To a sealed vial was added 1-(6-bromo-2-methyl-3,4-dihydroquinolin-1(2H)-yl)ethan-1-one (S2.2) (241 mg, 0.90 mmol), (4-(aminomethyl)phenyl)boronic acid (160 mg, 1.06 mmol) and K₂CO₃ (375 mg, 2.71 mmol) in a mixture of anhydrous dioxane and distilled water (4:1, 10 mL). A stream of nitrogen gas was bubbled through this suspension for 15 min. This suspension was then added PdCl₂(dppf) (47 mg, 0.06 mmol) and a stream of nitrogen gas was bubbled through this suspension for further 15 min. This reaction mixture was heated at 120° C. under nitrogen and stirred for 4 hr. This reaction mixture was then cooled to room temperature, added distilled water (50 mL) and extracted with DCM (3×50 mL). The organic extracts were then combined, washed with brine, dried over MgSO₄ and the solvent removed. The resulting crude material was purified by flash column chromatography (0-15% MeOH in DCM) to give the title compound as a brown oil that crystallized on standing (235 mg, 89%). ¹H NMR (400 MHz, CDCl₃) δ 7.56 (d, J=8.0 Hz, 2H), 7.43-7.38 (m, 4H), 7.20 (br s, 1H), 4.84 (br s, 1H), 3.93 (s, 2H), 2.71-2.54 (m, 2H), 2.37 (m, 1H), 2.19 (s, 3H), 1.40 (br s, 1H), 1.16 (d, J=6.5 Hz, 3H). LCMS: (0-100% Acetonitrile over 5 min, 0.1% formic acid throughout) Rf=2.804 min, (ESI) m/z: 295.0 (M+H, 100%).

### 1-(4-(1-Acetyl-2-methyl-1,2,3,4-tetrahydroquinolin-6-yl)phenyl)ethan-1-one (S2.5)

To a solution of 1-(6-bromo-2-methyl-3,4-dihydroquinolin-1(2H)-yl)ethan-1-one (S2.2) (540 mg, 2.01 mmol) in a mixture of anhydrous DME (8 mL) and distilled water (2 mL) was added 4-acetylphenyl boronic acid (397 mg, 2.42 mmol) and K₂CO₃ (835 mg, 6.04 mmol). A stream of nitrogen gas was bubbled through this suspension for 15 min. This suspension was then added PdCl₂(dppf) (106 mg, 0.20 mmol) and a stream of nitrogen gas was bubbled through this suspension for further 15 min. This reaction mixture was heated at 100° C. under a nitrogen atmosphere and stirred for 4 hr. This reaction mixture was then cooled to room temperature, added distilled water (50 mL) and extracted with DCM (3×50 mL). The organic extracts were then combined, washed with brine, dried over MgSO₄ and the solvent removed. The resulting crude material was purified by flash column chromatography (0-50% ethyl acetate in petroleum spirit), with the solvent removed in vacuo, the product was obtained as an yellow oil that solidified on standing as a pale yellow powder (564 mg, 91%). ¹H NMR (400 MHz, CDCl₃) δ 8.02 (app. dt, 2H), 7.67 (app. dt, 2H), 7.47 (dd, J=8.0 Hz, 2.0 Hz, 1H), 7.43 (sd, J=1.6 Hz, 1H), 7.25 (br s, 1H), 4.83 (br s, 1H), 2.74-2.56 (m, 2H), 2.63 (s, 3H), 2.41-2.33 (m, 1H), 1.42 (br s, 1H), 1.16 (d, J=6.5 Hz, 3H). LCMS: (0-100% Acetonitrile over 10 min, 0.1% formic acid throughout) Rf=5.867 min, (ESI) m/z: 308.2 (M+H, 100%)

### 5-(1-Acetyl-2-methyl-1,2,3,4-tetrahydroquinolin-6-yl)thiophene-2-carbaldehyde (S2.6)

To a solution of 1-(6-bromo-2-methyl-3,4-dihydroquinolin-1(2H)-yl)ethan-1-one (S2.2) (380 mg, 1.42 mmol) in anhydrous DME (8 mL) and distilled water (2 mL) was added 5-formyl-2-thiophene boronic acid (350 mg, 2.24 mmol) and K₂CO₃ (620 mg, 4.49 mmol). A stream of nitrogen gas was bubbled through this suspension for 15 min. This suspension was then added PdCl₂(dppf) (80 mg, 0.11 mmol) and a stream of nitrogen gas was bubbled through this suspension for further 15 min. This reaction mixture was heated at 100° C. under nitrogen and stirred for 4 hr. This reaction mixture was then cooled to room temperature, added distilled water (50 mL) and extracted with DCM (3×50 mL). The organic extracts were then combined, washed with brine, dried over MgSO₄ and the solvent removed. The resulting crude material was purified by flash column chromatography (0-50% ethyl acetate in petroleum spirit), with the solvent removed in vacuo, the product was obtained as an orange oil. The oil was triturated with diethyl ether and petroleum spirit to give the title compound as a bright yellow powder (308 mg, 73%). ¹H NMR (400 MHz, CDCl₃) δ 9.88 (s, 1H), 7.73 (d, J=4.0 Hz, 1H), 7.51 (dd, J=8.1 Hz, 2.0 Hz, 1H), 7.46 (sd, J=1.8 Hz, 1H), 7.37 (d, J=3.9 Hz, 1H), 7.24 (br s, 1H), 4.79 (br s, 1H), 2.73-2.55 (m, 2H), 2.39-2.31 (m, 1H), 2.19 (s, 3H), 1.47-1.40 (m, 1H), 1.15 (d, J=6.5 Hz, 3H). LCMS: (0-100% Acetonitrile over 10 min, 0.1% formic acid throughout) Rf=5.823 min, (ESI) m/z: 300.1 (M+H, 100%).

### tert-Butyl (2-((4-(1-acetyl-2-methyl-1,2,3,4-tetrahydroquinolin-6-yl)benzyl)amino)-2-ox-oethyl)carbamate (S3.1)

To a solution of 1-(6-(4-(aminomethyl)phenyl)-2-methyl-3,4-dihydroquinolin-1(2H)-yl)ethan-1-one (S2.4) (50 mg, 0.17 mmol) in DMF (3 mL) was added Boc-glycine (60 mg, 0.34 mmol), HCTU (141 mg, 0.34 mmol) and DIPEA (135 mg, 1.04 mmol). The solution was stirred at room temperature for 3 hr. The solution was then poured into distilled water (65 mL) and extracted with ethyl acetate (50 mL). Layers were separated after several hours. The organic layer was then washed successively with sat. NaHCO₃ solution (2×50 mL), brine (50 mL), dried over MgSO₄ and the solvent removed in vacuo. The resulting crude material was purified by flash column chromatography (0-45% ethyl acetate in petroleum spirit) to give the title compound as a yellow crystal (62 mg, 81%). ¹H NMR (400 MHz, CDCl₃) δ 7.49 (d, J=8.2 Hz, 2H), 7.36-7.29 (m, 4H), 7.14 (br s, 1H), 7.06 (br s, 1H), 5.55 (t, J=5.5 Hz, 1H), 4.78 (br s, 1H), 4.45 (d, J=5.7 Hz, 2H), 3.83 (d, J=4.3 Hz, 2H), 2.59 (m, 2H), 2.32 (m, 1H), 2.13 (s, 3H), 1.40 (s, 10H), 1.12 (d, J=6.4 Hz, 3H). LCMS: (0-100% Acetonitrile over 10 min, 0.1% formic acid throughout) Rf=5.700 min, (ESI) m/z: 452.3 (M+H, 100%).

### 1-(2-Methyl-6-(5-(piperazin-1-ylmethyl)thiophen-2-yl)-3,4-dihydroquinolin-1(2H)-yl)ethan-1-one (S4.1)

To a solution of 5-(1-acetyl-2-methyl-1,2,3,4-tetrahydroquinolin-6-yl)thiophene-2-carbaldehyde (S2.6) (57 mg, 0.19 mmol) in DCM (2 mL) was added piperazine (66 mg, 0.77 mmol) and sodium triacetoxyborohydride (81 mg, 0.31 mmol). The resulting suspension was stirred at room temperature under nitrogen for 9 hr. To the reaction mixture was added 10% NaOH (10 mL) solution and the aqueous layer was extracted by DCM (3×10 mL). The organic extracts were combined, washed with brine, dried over MgSO₄ and the solvent removed in vacuo. The resulting crude material was purified by flash column chromatography (0-10% MeOH in DCM) to give the title compound as a yellow semi-solid (44 mg, 63%). ¹H NMR (400 MHz, CDCl₃) δ 7.39 (dd, J=8.2 Hz, 1.8 Hz, 1H), 7.35 (s, 1H), 7.10 (app. d, 2H), 6.84 (d, J=3.5 Hz, 1H), 4.79 (br s, 1H), 3.67 (s, 2H), 2.91 (app t, 4H), 2.65-2.59 (m, 1H), 2.55-2.39 (m, 5H), 2.36-2.29 (m, 1H), 2.14 (s, 3H), 1.35 (br s, 1H), 1.11 (d, J=6.5 Hz, 3H). LCMS: (0-100% Acetonitrile over 10 min, 0.1% formic acid throughout) Rf=4.593 min, (ESI) m/z: 370.2 (M+H, 100%).

### 1-(6-(5-(Aminomethyl)thiophen-2-yl)-2-methyl-3,4-dihydroquinolin-1(2H)-yl)ethan-1-one (S4.3)

Step 1: To a solution of 5-(1-acetyl-2-methyl-1,2,3,4-tetrahydroquinolin-6-yl)thio-phene-2-carbaldehyde (S2.6) (210 mg, 0.53 mmol) in absolute ethanol (8 mL) was added a solution of hydroxylamine hydrochloride (120 mg, 1.73 mmol) in water (15 mL) and 1N sodium hydroxide (15 mL). The resulting mixture was allowed to stir at room temperature for 2 hr. The organic solvent was removed in vacuo and the remaining aqueous layer extracted by DCM (3×30 mL). The organic extracts were combined, washed with brine (30 mL), dried over MgSO₄ and the solvent removed in vacuo to a give the oxime intermediate (S4.2) as a yellow solid. Step 2: The yellow solid was re-dissolved in MeOH (8 mL) and to this solution was added ammonium formate (340 mg, 5.50 mmol) and zinc dust (350 mg, 5.35 mmol). The resulting suspension was stirred and heated to reflux under nitrogen atmosphere for 2 hr. The reaction mixture was allowed to cool to room temperature and the solid filtered. The organic solvent was concentrated and the resulting crude material purified by flash column chromatography (0-5% MeOH in DCM) to give the title product as a yellow oil (140 mg, 66% over two steps).

¹H NMR (400 MHz, CDCl₃) δ 7.31 (d, J=8.2 Hz, 1H), 7.28 (s, 1H), 7.04 (app d, 2H), 6.79 (app d, 1H), 4.71 (br s, 1H), 3.95 (s, 2H), 2.49 (m, 2H), 2.24 (m, 1H), 2.07 (s, 3H), 1.28 (br s, 1H), 1.04 (d, J=6.4 Hz, 3H). LCMS: (0-100% Acetonitrile over 10 min, 0.1% formic acid throughout) Rf=4.532 min, (ESI) m/z: 301.2 (M+H, 100%).

### tert-Butyl (2-(((5-(1-acetyl-2-methyl-1,2,3,4-tetrahydroquinolin-6-yl)thiophen-2-yl)methyl)amino)-2-oxoethyl)carbamate (S4.4)

To a solution of 1-(6-(5-(aminomethyl)thiophen-2-yl)-2-methyl-3,4-dihydroquinolin-1(2H)-yl)ethan-1-one (S4.3) (75 mg, 0.25 mmol) in DMF (3 mL) was added Boc-glycine (88 mg, 0.50 mmol), HCTU (207 mg, 0.50 mmol), DIPEA (197 mg, 1.52 mmol). The resulting solution was stirred at room temperature for 3 hr and then poured into distilled water (40 mL). The mixture was then extracted with ethyl acetate (100 mL). The organic extract was washed with sat. NaHCO₃ solution (2×100 mL) and brine (100 mL), dried over MgSO₄ and the solvent removed in vacuo. The resulting crude material was then purified by flash column chromatography (0-50% ethyl acetate in DCM) to give the title compound as a yellow solid (87 mg, 76%). ¹H NMR (400 MHz, CDCl₃) δ 7.36 (dd, J=8.2 Hz, 2.0 Hz), 7.32 (s, 1H), 7.12 (br s, 1H), 7.08 (d, J=3.6 Hz, 1H), 6.90 (d, J=3.5 Hz, 1H), 6.86 (br s, 1H), 5.32 (br s, 1H), 4.78 (br s, 1H), 4.59 (d, J=5.8 Hz, 2H), 3.83 (d, J=5.4 Hz, 2H), 2.57 (m, 2H), 2.33 (m, 1H), 2.14 (s, 3H), 1.42 (s, 9H), 1.37 (br s, 1H), 1.12 (d, J=6.5 Hz, 3H). LCMS: (0-100% Acetonitrile over 10 min, 0.1% formic acid throughout) Rf=5.672 min, (ESI) m/z: 458.2 (M+H, 100%).

### 1-(4-(1-Acetyl-2-methyl-1,2,3,4-tetrahydroquinolin-6-yl)phenyl)-2-bromoethan-1-one (S5.1)

To a solution of 1-(4-(1-acetyl-2-methyl-1,2,3,4-tetrahydroquinolin-6-yl)phenyl)ethan-1-one (S2.5) (420 mg, 1.37 mmol) in acetonitrile (6 mL) was added p-toluenesulfonic acid (479 mg, 2.78 mmol) and NBS (255 mg, 1.43 mmol). This solution was then heated to reflux for 30 min under nitrogen. The solution was cooled to room temperature and the solvent removed. The crude material was re-dissolved in DCM (50 mL) and washed successively with distilled water (50 mL), 10% NaOH solution (3×50 mL), brine (50 mL) and dried over MgSO₄. The solvent was then removed in vacuo to give the title compound as a bright yellow solid (527.6 mg) that was contaminated by the di-brominated product (<10% determined by ¹H NMR integration ratio). The crude product was used in the next step without further purification. ¹H NMR (400 MHz, CDCl₃) δ 8.00 (app. dt, 2H), 7.64 (app. dt, 2H), 7.42 (dd, J=8.2 Hz, 1.8 Hz, 1H), 7.37 (sd, J=1.6 Hz, 1H), 7.20 (br s, 1H), 4.76 (br s, 1H), 4.41 (s, 2H), 2.60 (m, 2H), 2.34-2.29 (m, 1H), 2.14 (s, 3H), 1.37 (br s, 1H), 1.10 (d, J=6.5 Hz, 3H). LCMS: (0-100% Acetonitrile over 5 min, 0.1% formic acid throughout) Rf=3.449 min, (ESI) m/z: 385.8 (M+H, 100%).

### tert-Butyl 4-(2-(4-(1-acetyl-2-methyl-1,2,3,4-tetrahydroquinolin-6-yl)phenyl)-2-ox-oethyl)piperazine-1-carboxylate (S5.2)

To a solution of 1-(4-(1-acetyl-2-methyl-1,2,3,4-tetrahydroquinolin-6-yl)phenyl)-2-bromoethan-1-one (S5.1) (135 mg of crude starting material, 0.35 mmol) in DCM (5 mL) was added DIPEA (93 mg, 0.72 mmol) and Boc-piperazine (130 mg, 0.70 mmol). The solution was then stirred at room temperature for 1 hr. The reaction mixture was then diluted with DCM (30 mL) and washed with distilled water (3×30 mL), brine (30 mL), dried over MgSO₄ and the solvent removed in vacuo. The resulting crude material was purified by flash column chromatography (0-80% ethyl acetate in petroleum spirit) to give the title compound as a pale yellow powder (129 mg, 75%). ¹H NMR (400 MHz, CDCl₃) δ 8.04 (d, J=8.2 Hz, 211), 7.65 (d, J=8.2 Hz, 2H), 7.44 (d, J=8.2 Hz, 1H), 7.41 (s, 1H), 7.24 (br s, 1H), 4.80 (br s, 1H), 3.84 (s, 2H), 3.49 (s, 4H), 2.73-2.66 (m, 1H), 2.55 (br s, 5H), 2.34 (m, 1H), 2.17 (s, 3H), 1.43 (s, 10H), 1.13 (d, J=6.4 Hz, 31-1). LCMS: (0-100% Acetonitrile over 10 min, 0.1% formic acid throughout) Rf=3.020 min, (ESI) m/z: 492.0 (M+H, 100%).

### 1-(4-(1-Acetyl-2-methyl-1,2,3,4-tetrahydroquinolin-6-yl)phenyl)-2-aminoethan-1-one (S5.5)

Step 1: To a solution of 1-(4-(1-acetyl-2-methyl-1,2,3,4-tetrahydroquinolin-6-yl)phenyl)-2-bromoethan-1-one (S5.1) (590 mg, 1.53 mmol) in DMF (6 mL) was added sodium azide (121 mg, 1.86 mmol). The resulting suspension was stirred at room temperature for 2 hr. The mixture was then poured into distilled water (50 mL) and extracted with ethyl acetate (3×50 mL). The organic extracts were then combined and washed successively with sat. NaHCO₃ solution (2×50 mL) and brine (50 mL), dried over MgSO₄ and the solvent removed in vacuo to give 1-(4-(1-acetyl-2-methyl-1,2,3,4-tetrahydroquinolin-6-yl)phenyl)-2-azidoethan-1-one (S5.4) as bright yellow powder (459 mg). The crude material was then used in the next step without further purification. Step 2: 1-(4-(1-acetyl-2-methyl-1,2,3,4-tetrahydroquinolin-6-yl)phenyl)-2-azidoethan-1-one (300 mg) obtained from last step was dissolved in MeOH (10 mL) and added Pd/C (10%) under an inert environment. The reaction vessel was then ex changed to hydrogen atmosphere and the reaction mixture stirred at room temperature for 4 hr. The Pd/C was then filtered, the mother liquors was then concentrated. The resulting crude material was then re-dissolved in DCM (20 mL) and added 1N HCl (20 mL). The layers were separated and the aqueous layer was then basified by 10% NaOH solution and extracted by DCM (2×20 mL). The organic extracts were combined, washed with brine, dried over MgSO₄ and the solvent removed to give the title compound as an orange-red solid (38 mg, 12% over 2 steps). ¹H NMR (400 MHz, CDCl₃) δ 9.15 (s, 1H), 8.17 (d, J=8.5 Hz, 2H), 7.77 (d, J=8.6 Hz, 2H), 7.52 (dd, J=8.2 Hz, 1.9 Hz, 1H), 7.49 (s, 1H), 7.26 (br s, 1H), 4.85 (br s, 1H), 2.76-2.58 (m, 2H), 2.43-2.36 (m, 1H), 2.22 (s, 3H), 1.43 (br s, 1H), 1.18 (d, J=6.4 Hz, 3H). LCMS: (0-100% Acetonitrile over 5 min, 0.1% formic acid throughout) Rf=2.848 min, (ESI) m/z: 323.0 (M+H, 100%).

### 1-(6-(4-(4-(6-(2-Aminopyrimidin-5-yl)-8-morpholinoimidazo[1,2-a]pyrazine-2-carbonyl)piperazine-1-carbonyl)phenyl)-2-methyl-3,4-dihydroquinolin-1(2H)-yl)ethan-1-one [Cpd 001]

Step 1: A solution of tert-butyl 4-(6-(2-aminopyrimidin-5-yl)-8-morpholinoimidazo[1,2-a]pyrazine-2-carbonyl)piperazine-1-carboxylate (S1.5) (60 mg, 0.12 mmol) in a mixture of DCM and TFA (1:1, 4 mL) was stirred at room temperature for 1 hr. The solvent was then removed by a stream of nitrogen gas to give a bright yellow residue (S1.6). This crude material was used in the next step with no further purification. Step 2: The residue was re dissolved in DMF (1 mL) and added DIPEA (90 mg, 0.70 mmol) dropwise until no smoke evolved. The solution was stirred for 10 min, then added 4-(1-acetyl-2-methyl-1,2,3,4-tetrahy-droquinolin-6-yl)benzoic acid (S2.3) (54 mg, 0.17 mmol), HCTU (145 mg, 0.35 mmol) and another portion of DIPEA (90 mg, 0.70 mmol). This solution was then stirred at room temperature for 4 hr and then poured into distilled water (50 mL). The precipitate was collected and purified by flash column chromatography (0-10% MeOH in DCM) to give the title compound as a yellow powder (74 mg, 89%). ¹H NMR (400 MHz, CDCl₃) δ 8.80 (s, 2H), 8.09 (s, 1H), 7.81 (s, 1H), 7.66 (d, J=8.3 Hz, 2H), 7.53 (d, J=8.4 Hz, 2H), 7.45 (dd, J=8.1 Hz, 1.9 Hz, 1H), 7.41 (sd, J=1.5 Hz, 1H), 5.21 (s, 2H), 4.83 (br s, 1H), 4.34 (s, 6H), 3.94-3.58 (m, 10H), 2.74-2.57 (m, 2H), 2.42-2.34 (m, 1H, H15), 2.21 (s, 3H), 1.43 (br s, 1H), 1.17 (d, J=6.5 Hz, 3H). LCMS: (0-100% Acetonitrile over 5 min, 0.1% formic acid throughout) Rf=3.170 min, (ESI) m/z: 702.0 (M+H, 100%). HRMS (ESI+) calcd for [C₃₈H₄₀N₁₀O₄+H]⁺701.3307, found 701.3322.

### N-(4-(1-Acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide [Cpd 002]

To a solution of 1-(6-(4-(aminomethyl)phenyl)-2-methyl-3,4-dihydroquinolin-1(2H)-yl)ethan-1-one (S2.4) (115 mg, 0.39 mmol) in DMF (4 mL) was added lithium 6-(2-aminopy-rimidin-5-yl)-8-morpholinoimidazo[1,2-a]pyrazine-2-carboxylate (S1.4) (200 mg, 0.59 mmol), HCTU (485 mg, 1.17 mmol) and DIPEA (152 mg, 1.18 mmol). The reaction mixture was stirred at room temperature for 4 hr. The reaction mixture was then poured into distilled water (50 mL). The precipitate was collected and purified by flash column chromatography (0-4% MeOH in DCM) to give the title compound as an off-white powder (101 mg, 42%). ¹H NMR (400 MHz, CDCl₃) δ 8.81 (s, 2H), 8.14 (s, 1H), 7.83 (s, 1H), 7.60-7.57 (m, 2H), 7.54 (t, J=6.0 Hz, 1H), 7.46 (d, J=8.4 Hz, 2H), 7.42 (dd, J=8.2, 1.7 Hz, 1H), 7.38 (sd, J=1.9 Hz, 1H), 7.21 (s, 1H), 5.20 (s, 2H), 4.84 (s, 1H), 4.75 (d, J=6.3 Hz, 2H), 4.34 (t, J=4.6 Hz, 4H), 3.88 (t, J=4.8 Hz, 4H), 2.72-2.54 (m, 2H), 2.37 (ddt, J=12.9, 7.7, 5.1 Hz, 1H), 2.19 (s, 3H), 1.64 (s, 3H), 1.40 (br s, 1H), 1.16 (d, J=6.5 Hz, 3H). LCMS: (0-100% Acetonitrile over 10 min, 0.1% formic acid throughout) Rf=6.050 min, (ESI) m/z: 618.3 (M+H, 100%). HRMS (ESI+) calcd for [C₃₄H₃₅N₉O₃+H]+618.2936, found 618.2926.

### N-(2-((4-(1-Acetyl-2-methyl-1,2,3,4-tetrahydroquinolin-6-yl)benzyl)amino)-2-oxoethyl)-6-(2-aminopyrimidin-5-yl)-8-morpholinoimidazo[1,2-a]pyrazine-2-carboxamide [Cpd 003]

Step 1: A solution of tert-butyl (2-((4-(1-acetyl-2-methyl-1,2,3,4-tetrahydroquinolin-6-yl)benzyl)amino)-2-oxoethyl)carbamate (S3.1) (60 mg, 0.13 mmol) in DCM:TFA mixture (1:1, 3 mL) was stirred at room temperature for 1 hr. The solvent was then removed by a stream of nitrogen gas to give a crude oil (S3.2). Step 2: The crude material was re-dissolved in DMF (3 mL) and added dropwise DIPEA until no smoke was evolved. To this solution was added lithium 6-(2-aminopyrimidin-5-yl)-8-morpholinoimidazo[1,2-a]pyrazine-2-carboxylate (S1.4) (93 mg, 0.27 mmol), HCTU (110 mg, 0.27 mmol) and DIPEA (105 mg, 0.81 mmol). The solution was stirred at room temperature for 4 hr and then poured into distilled water (75 mL). The precipitate was collected and purified by flash column chromatography (0-7% MeOH in DCM). The title compound was obtained as a yellow powder (53 mg, 59%). ¹H NMR (400 MHz, 0.6 mL CDCl₃+3 drops of MeOD) δ 8.76 (s, 2H), 8.10 (s, 1H), 8.03 (t, J=5.6 Hz, 1H), 7.84 (s, 1H), 7.53 (d, J=8.3 Hz, 2H), 7.45 (t, J=5.7 Hz, 1H), 7.39 (dd, J=8.2, 1.8 Hz, 1H), 7.36-7.34 (m, 3H), 7.17 (br s, 1H), 4.80 (br s, 1H), 4.50 (d, J=5.6 Hz, 2H), 4.36 (t, J=4.5 Hz, 4H), 4.15 (d, J=5.6 Hz, 2H), 3.89 (t, J=4.8 Hz, 4H), 2.70-2.55 (m, 2H), 2.40-2.32 (m, 1H), 2.17 (s, 3H), 1.38 (br s, 1H), 1.14 (d, J=6.5 Hz, 3H). LCMS: (0-100% Acetonitrile over 5 min, 0.1% formic acid throughout) Rf=3.119 min, (ESI) m/z: 674.9 (M+H, 100%). HRMS (ESI+) calcd for [C₃₆H₃₈N₁₀O₄+H]⁺675.3150, found 675.3167.

### 1-(6-(5-((4-(6-(2-Aminopyrimidin-5-yl)-8-morpholinoimidazo[1,2-a]pyrazine-2-carbonyl)piperazin-1-yl)methyl)thiophen-2-yl)-2-methyl-3,4-dihydroquinolin-1(2H)-yl)ethan-1-one [Cpd 004]

To a solution of 1-(2-methyl-6-(5-(piperazin-1-ylmethyl)thiophen-2-yl)-3,4-dihydro-quinolin-1(2H)-yl)ethan-1-one (S4.1) (40 mg, 0.11 mmol) in DMF (1 mL) was added lithium 6-(2-aminopyrimidin-5-yl)-8-morpholinoimidazo[1,2-a]pyrazine-2-carboxylate (S1.4) (74 mg, 0.22 mmol), HCTU (90 mg, 0.22 mmol) and DIPEA (91 mg, 0.70 mmol). The resulting suspension was stirred at room temperature for 3 hr and then poured into distilled water (75 mL) and left on standing for 15 min before filtering. The precipitate was collected and purified by flash column chromatography (0-3% MeOH in DCM) to give the title product as a pale yellow powder (32 mg, 43%). ¹H NMR (400 MHz, CDCl₃) δ 8.79 (s, 2H), 8.03 (s, 1H), 7.79 (s, 1H), 7.43 (dd, J=8.2, 2.0 Hz, 1H), 7.38 (sd, J=1.6 Hz, 1H), 7.14 (app. d, 2H), 6.89 (d, J=3.6 Hz, 1H), 5.20 (s, 2H), 4.81 (br s, 1H), 4.35-4.25 (m, 6H), 3.86, (app t, 6H), 2.68-2.51 (m, 6H), 3.77 (s, 2H), 2.35 (m, 1H), 2.17 (s, 3H), 1.38 (br s, 1H), 1.14 (d, J=6.5 Hz, 3H). LCMS: (0-100% Acetonitrile over 10 min, 0.1% formic acid throughout) Rf=4.926 min, (ESI) m/z: 693.3 (M+H, 100%). HRMS (ESI+) calcd for [C₃₆H₄₀N₁₀O₃S+H]⁺693.3078, found 693.3091.

### N-((5-(1-Acetyl-2-methyl-1,2,3,4-tetrahydroquinolin-6-yl)thiophen-2-yl)methyl)-6-(2-aminopyrimidin-5-yl)-8-morpholinoimidazo[1,2-a]pyrazine-2-carboxamide [Cpd 005]

To solution of 1-(6-(5-(aminomethyl)thiophen-2-yl)-2-methyl-3,4-dihydroquinolin-1(2H)-yl)ethan-1-one (S4.3) (65 mg, 0.22 mmol) in DMF (2 mL) was added lithium 6-(2-ami-nopyrimidin-5-yl)-8-morpholinoimidazo[1,2-a]pyrazine-2-carboxylate (S1.4) (110 mg, 0.32 mmol), HCTU (180 mg, 0.44 mmol) and DIPEA (171 mg, 1.32 mmol). The resulting suspension was stirred at room temperature for 4 hr and then poured into distilled water (75 mL). The precipitate was collected by filtration and then purified by flash column chromatography (0-10% MeOH in DCM) to give the title compound as pale yellow powder (67 mg, 50%). ¹H NMR (400 MHz, 0.5 mL CDCl₃+2 drops of MeOD) δ 8.74 (s, 2H), 8.14 (s, 1H), 7.86 (s, 1H), 7.37 (dd, J=8.2, 2.0 Hz, 1H), 7.32 (sd, J=0.7 Hz, 1H), 7.11 (d, J=3.6 Hz, 1H), 7.07 (br s, 1H), 6.99 (d, J=3.6, 1H), 4.79 (s, 2H), 4.74 (br s, 1H), 4.29 (app t, 4H), 3.85 (app t, 4H), 2.67 (br s, 1H), 2.55-2.45 (m, 1H), 2.31 (m, 1H), 2.12 (s, 3H), 1.33 (br s, 1H), 1.09 (d, J=6.4 Hz, 3H). LCMS: (0-100% Acetonitrile over 10 min, 0.1% formic acid throughout) Rf=5.852 min, (ESI) m/z: 624.3 (M+H, 100%). HRMS (ESI+) calcd for [C₃₂H₃₃N₉O₃S+H]⁺624.2500, found 624.2511.

### N-(2-(((5-(1-Acetyl-2-methyl-1,2,3,4-tetrahydroquinolin-6-yl)thiophen-2-yl)methyl)amino)-2-oxoethyl)-6-(2-aminopyrimidin-5-yl)-8-morpholinoimidazo[1,2-a]pyrazine-2-carboxamide [Cpd 006]

Step 1: A solution of tert-butyl (2-(((5-(1-acetyl-2-methyl-1,2,3,4-tetrahydroquinolin-6-yl)thiophen-2-yl)methyl)amino)-2-oxoethyl)carbamate (S4.4) (60 mg, 0.13 mmol) in a mixture of TFA and DCM (1:1, 3 mL) was stirred at room temperature for 1 hr. The solvent was removed by a stream of nitrogen gas to give a crude oil (S4.5). Step 2: The oil mixture was re dissolved in DMF (3 mL) added DIPEA dropwise until no smoke evolved. To this solution, was then added lithium 6-(2-aminopyrimidin-5-yl)-8-morpholinoimidazo[1,2-a]pyrazine-2-carboxylate (S1.4) (91 mg, 0.27 mmol), HCTU (108 mg, 0.26 mmol) and another portion of DIPEA (103 mg, 0.80 mmol). The solution was stirred at room temperature for 2 hr and then poured into distilled water (75 mL). The precipitate was collected and purified by flash column chromatography (0-8% MeOH in DCM) to give the title compound as a pale yellow powder (56 mg, 63%). ¹H NMR (400 MHz, 0.5 mL CDCl₃₊₂ drops of MeOD) δ 8.73 (s, 2H), 8.07 (s, 1H), 8.00 (t, J=5.6 Hz, 1H), 7.82 (s, 1H), 7.60 (t, J=5.2 Hz, 1H), 7.34 (dd, J=8.3, 2.0 Hz, 1H), 7.30 (s, 1H), 7.07 (app. d, 2H), 6.90 (d, J=3.6 Hz, 1H), 4.74 (br s, 1H), 4.58 (d, J=5.6 Hz, 2H), 4.32 (app t, 4H), 4.10 (d, J=5.6 Hz, 2H), 3.86 (app t, 4H), 2.58-2.52 (m, 2H), 2.34-2.28 (m, 1H), 2.12 (s, 3H), 1.32 (br s, 1H), 1.09 (d, J=6.5 Hz, 3H). LCMS: (0-100% Acetonitrile over 10 min, 0.1% formic acid throughout) Rf=5.504 min, (ESI) m/z: 681.3 (M+H, 100%). HRMS (ESI+) calcd for [C₃₄H₃₆N₁₀O₄S+H]⁺681.2714, found 681.2728.

### 1-(4-(1-Acetyl-2-methyl-1,2,3,4-tetrahydroquinolin-6-yl)phenyl)-2-(4-(6-(2-aminopyrim-idin-5-yl)-8-morpholinoimidazo[1,2-a]pyrazine-2-carbonyl)piperazin-1-yl)ethan-1-one [Cpd 007]

Step 1: A solution of tert-butyl 4-(2-(4-(1-acetyl-2-methyl-1,2,3,4-tetrahydroquinolin-6-yl)phenyl)-2-oxoethyl)piperazine-1-carboxylate (S5.2) (56 mg, 0.11 mmol) in a mixture of TFA and DCM (1:1, 3 mL) was stirred at room temperature for 1 hr. The solvent was then removed by a stream of nitrogen gas and gave the unprotected amine as a crude oil (S5.3). Step 2: The crude amine was re-dissolved in DMF (3 mL) and added DIPEA dropwise until no smoke evolved. To this solution was added lithium 6-(2-aminopyrimidin-5-yl)-8-morpholinoimidazo[1,2-a]pyrazine-2-carboxylate (S1.4) (78 mg, 0.23 mmol), HCTU (94 mg, 0.23 mmol) and another portion of DIPEA (93 mg, 0.72 mmol). The resulting solution was stirred at room temperature for 3 hr and then poured into distilled water (75 mL). The precipitate was collected and purified by flash column chromatography (0-7% MeOH in DCM) to give the title compound as a yellow solid (56 mg, 69%). ¹H NMR (400 MHz, CDCl₃) δ 8.79 (s, 2H), 8.08 (app. dt, 2H), 8.05 (s, 1H), 7.80 (s, 1H), 7.68 (app. dt, 2H), 7.48 (dd, J=8.2 Hz, 2.0 Hz, 1H), 7.43 (sd, J=1.6 Hz, 1H), 7.27 (br s, 1H), 5.20 (s, 2H), 4.83 (br s, 1H), 4.35 (s, 6H), 3.92 (s, 4H), 3.87 (app t, 4H), 2.76-2.64 (m, 6H), 2.41-2.35 (m, 1H), 2.21 (s, 3H), 1.43 (br s, 1H), 1.17 (d, J=6.5 Hz, 3H). LCMS: (0-100% Acetonitrile over 10 min, 0.1% formic acid throughout) Rf=4.994 min, (ESI) m/z: 715.3 (M+H, 100%). HRMS (ESI+) calcd for [C₃₉H₄₂N₁₀O₄+H]⁺715.3467, found 715.3463.

### N-(2-(4-(1-Acetyl-2-methyl-1,2,3,4-tetrahydroquinolin-6-yl)phenyl)-2-oxoethyl)-6-(2-aminopyrimidin-5-yl)-8-morpholinoimidazo[1,2-a]pyrazine-2-carboxamide [Cpd 008]

To a solution of 1-(4-(1-acetyl-2-methyl-1,2,3,4-tetrahydroquinolin-6-yl)phenyl)-2-aminoethan-1-one (S5.5) (35 mg, 0.11 mmol) in DMF (3 mL) was added lithium 6-(2-amino-pyrimidin-5-yl)-8-morpholinoimidazo[1,2-a]pyrazine-2-carboxylate (S1.4) (76 mg, 0.22 mmol), HCTU (90 mg, 0.22 mmol) and DIPEA (93 mg, 0.72 mmol). The resulting suspension was stirred at room temperature for 3 hr and poured into distilled water (50 mL). The precipitate was collected and purified by reverse phase prep-HPLC (0-100% Acetonitrile in water+0.1% TFA, flow rate: 10 mL/min) to give the title compound as an off-white powder (4 mg, 6%). ¹H NMR (400 MHz, CDCl₃) δ 8.87 (s, 2H), 8.14-8.11 (m, 3H), 7.86 (s, 1H), 7.75 (d, J=8.6 Hz, 2H), 7.50 (dd, J=8.1, 1.8 Hz, 1H), 7.46 (sd, J=1.8 Hz, 1H), 6.66 (br s, 1H), 5.34 (s, 2H), 5.02 (d, J=4.8, 2H), 4.84 (s, 1H), 4.44-4.38 (m, 4H), 3.95 (app t, 4H), 2.77-2.58 (m, 2H), 2.43-2.33 (m, 1H), 2.22 (s, 3H), 1.45 (br s, 1H), 1.18 (d, J=6.5 Hz, 3H). LCMS: (0-100% Acetonitrile over 5 min, 0.1% formic acid throughout) Rf=3.252 min, (ESI) m/z: 646.0 (M+H, 100%). HRMS (ESI+) calcd for [C₃₅H₃₅N₉O₄+H]⁺646.2885, found 646.2893.

### N-(2-Aminoethyl)-6-bromo-8-morpholinoimidazo[1,2-a]pyrazine-2-carboxamide (S8.1)

A solution of ethyl 6-bromo-8-morpholinoimidazo[1,2-a]pyrazine-2-carboxylate (S1.2) (345 mg, 0.97 mmol) in a mixture of DCM and ethylenediamine (3:1, 16 mL) was stirred at room temperature for 16 hr. The reaction mixture was added 10% NaOH solution (50 mL) and extracted with DCM (3×50 mL). The organic extracts were combined, washed with brine, dried over MgSO₄ and the solvent removed to give the title compound as a yellow solid (312 mg, 87%). The crude material was used in the next step without further purification. ¹H NMR (400 MHz, CDCl₃) δ 7.98 (s, 1H), 7.62 (s, 1H), 7.42 (t, J=5.8 Hz, 1H), 4.33 (s, 4H), 3.86 (app t, 4H), 3.53 (app. q, 2H), 2.96 (t, J=6.0 Hz, 2H). LCMS: (0-100% Acetonitrile over 5 min, 0.1% formic acid throughout) Rf=2.793 min, (ESI) m/z: 368.8 (M+H, 100%).

### N-(2-(4-(1-Acetyl-2-methyl-1,2,3,4-tetrahydroquinolin-6-yl)benzamido)ethyl)-6-bromo-8-morpholinoimidazo[1,2-a]pyrazine-2-carboxamide (S8.2)

To a solution of N-(2-aminoethyl)-6-bromo-8-morpholinoimidazo[1,2-a]pyrazine-2-carboxamide (S8.1) (150 mg, 0.41 mmol) in DMF (4 mL) was added 4-(1-acetyl-2-methyl-1,2,3,4-tetrahydroquinolin-6-yl)benzoic acid (S2.3) (151 mg, 0.49 mmol), HCTU (336 mg, 0.81 mmol) and DIPEA (300 mg, 2.32 mmol). This reaction mixture was then stirred at room temperature for 4 hr. The reaction mixture was poured into distilled water (75 mL). The precipitate was collected and purified by flash column chromatography (0-3% MeOH in DCM) to give the title compound as a white powder (239 mg, 89%). ¹H NMR (400 MHz, CDCl₃) δ 7.98 (s, 1H), 7.88 (d, J=8.5 Hz, 2H), 7.78 (br t, J=5.4 Hz, 1H), 7.65 (d, J=8.6 Hz, 2H), 7.59 (s, 1H), 7.45 (dd, J=8.2, 1.9 Hz, 1H), 7.41 (s, 1H), 7.33 (br t, J=5.2 Hz, 1H), 7.23 (br s, 1H), 4.83 (br s, 1H), 4.35 (s, 4H), 3.86 (app t, J=4.8 Hz, 4H), 3.75 (s, 4H), 2.74-2.56 (m, 2H), 2.41-2.34 (m, 1H), 2.20 (s, 3H), 1.42 (br s, 1H), 1.16 (d, J=6.5 Hz, 3H). LCMS: (0-100% Acetonitrile over 5 min, 0.1% formic acid throughout) Rf=6.049 min, (ESI) m/z: 660.2 (M+H, 100%).

### N-(2-(4-(1-Acetyl-2-methyl-1,2,3,4-tetrahydroquinolin-6-yl)benzamido)ethyl)-6-(2-ami-nopyrimidin-5-yl)-8-morpholinoimidazo[1,2-a]pyrazine-2-carboxamide [Cpd 009]

To a sealed vial was added N-(2-(4-(1-acetyl-2-methyl-1,2,3,4-tetrahydroquinolin-6-yl)benzamido)ethyl)-6-bromo-8-morpholinoimidazo[1,2-a]pyrazine-2-carboxamide (S8.2) (100 mg, 0.15 mmol), 2-aminopyrimidine-5-boronic acid (32 mg, 0.23 mmol) and K₂CO₃ (63 mg, 0.46 mmol) in a mixture of anhydrous DME and distilled water (4:1, 10 mL). A stream of nitrogen gas was bubbled through this suspension for 15 min. To this suspension was then added PdCl₂(dppf) (8 mg, 0.01 mmol) and a stream of nitrogen gas was bubbled through this suspension for further 15 min. The reaction mixture was heated at 100° C. under nitrogen and stirred for 4 hr. This reaction mixture was then cooled to room temperature, added distilled water (30 mL) and extracted with a mixture of DCM and 2-propanol (3:1, 3×50 mL). The organic extracts were then combined, washed with brine, dried over MgSO₄ and the solvent removed. The resulting crude material was purified by flash column chromatography (0-8% MeOH in DCM) and then triturated with MeOH to give the title compound as an off-white powder (92 mg, 90%). ¹H NMR (400 MHz, 0.5 mL of CDCl₃+2 drops of MeOD) δ 8.73 (s, 2H), 8.08 (s, 1H), 7.86 (d, J=8.2 Hz, 2H), 7.82 (s, 1H), 7.62 (d, J=8.2 Hz, 2H), 7.42 (dd, J=8.2 Hz, 1.9 Hz, 1H), 7.38 (s, 1H), 7.20 (br s, 1H), 4.77 (br s, 1H), 4.34 (app t, 4H), 3.87 (app t, 4H), 3.67 (br s, 4H), 2.71-2.64 (m, 1H), 2.58-2.51 (m, 1H), 2.38-2.30 (m, 1H), 2.15 (s, 3H), 1.37 (br s, 1H), 1.12 (d, J=6.5 Hz, 3H). LCMS: (0-100% Acetonitrile over 5 min, 0.1% formic acid throughout) Rf=3.115 min, (ESI) m/z: 674.9 (M+H, 100%). HRMS (ESI+) calcd for [C₃₆H₃₈N₁₀O₄+H]⁺675.3150, found 675.3168.

### (S)-2-(1,3-Dioxoisoindolin-2-yl)-4-methylpentanoic acid (S9.1)

To a mixture of phthalic anhydride (7.40 g, 50.0 mmol) and L-leucine (6.60 g, 50.3 mmol) in toluene (250 ml) was added TEA (506.0 mg, 5.0 mmol). The reaction mixture was refluxed for 3 h. The reaction mixture was then washed with 1N HCl solution (×2), dried over MgSO₄ and concentrated in vacuo to give the title compound as a white powder (12.95 g, 99%).

¹H NMR (401 MHz, CDCl₃) δ 7.89-7.82 (m, 2H), 7.76-7.70 (m, 2H), 5.00 (dd, J=11.5, 4.4 Hz, 1H), 2.37 (ddd, J=14.3, 11.5, 4.2 Hz, 1H), 1.95 (ddd, J=14.4, 10.2, 4.4 Hz, 1H), 1.58-1.41 (m, 1H), 0.94 (dd, J=8.6, 6.6 Hz, 6H). LCMS: (Method A) Rf=6.547 min, (ESI) m/z: (ESI) m/z: 284.1 ([M+Na]⁺, 100%), 262.1 ([M+H]⁺, 100%).

### 2-((S)-4-Methyl-1-((S)-2-methyl-3,4-dihydroquinolin-1(2H)-yl)-1-oxopentan-2-yl)isoin-doline-1,3-dione (S9.3)

Step 1: To a suspension of (S)-2-(1,3-dioxoisoindolin-2-yl)-4-methylpentanoic acid (S9.1) (13.50 g, 51.7 mmol) in a mixture of toluene and petroleum ether (1:1, 300 mL) was dropwise added oxalyl chloride (9.8 mL, 115 mmol) and DMF (100 μl). The reaction mixture was stirred at r.t o.n. The solvent was concentrated in vacuo to give the acid chloride intermediate (S9.2) as an oil. The residue was re-dissolved in DCM (200 mL) was added dropwise to a stirring solution of 2-methyl-1, 2, 3, 4-tetrahydroquinoline (12.0 g, 81.5 mmol) in DCM (200 ml) at −20° C. The reaction mixture was stirred at this temperature for 6 h. The reaction mixture was then washed with 1N HCl solution (2×150 ml). The aqueous layer was worked up separately to yield the (R)-enantiomerically enriched 2-methyl-1,2,3,4-tetrahydroquinoline (refer to the synthesis of S9.4 R enantiomer). The organic layer was washed successively with brine (1×200 ml), sat. bicarb solution (2×200 ml) and water (1×200 ml), dried over MgSO₄ and the solvent concentrated in vacuo. The residue was recrystallised from a mixture of PE and EA (5:2) to give the title compound as a white crystal (10.8 g 74%). ¹H NMR (401 MHz, CDCl₃) δ 7.89-7.82 (m, 2H), 7.75-7.68 (m, 2H), 7.58 (d, J=7.8 Hz, 1H), 7.38-7.31 (m, 1H), 7.26-7.21 (m, 2H), 5.73 (dd, J=12.7, 3.5 Hz, 1H), 4.81-4.66 (m, 1H), 2.75-2.57 (m, 2H), 2.53-2.35 (m, 2H), 1.36-1.18 (m, 2H), 1.10 (d, J=6.4 Hz, 3H), 0.80-0.69 (m, 1H), 0.67 (d, J=6.7 Hz, 3H), 0.34 (d, J=6.5 Hz, 3H). LCMS: (Method A) Rf=7.339 min, (ESI) m/z: 413.2 ([M+Na]⁺, 100%), 391.2 ([M+H]⁺, 100%).

### (S)-2-Methyl-1,2,3,4-tetrahydroquinoline (S9.4)

A solution of 2-((S)-4-methyl-1-((S)-2-methyl-3,4-dihydroquinolin-1(2H)-yl)-1-oxo-pentan-2-yl)isoindoline-1,3-dione (S9.3) (10.80 g, 27.66 mmol) in a mixture of glacial acetic acid and conc. HCl solution (1:1, 180 mL) was heated at 90° C. for 24 hr. The acetic acid was concentrated in vacuo. The remaining aqueous residue was basified to pH=14 with 10% NaOH solution and extracted with CHCl₃ (×3). The organic extracts were combined, washed with brine, dried over MgSO₄ and concentrated in vacuo to give the title compound a light yellow oil (3.79 g, 93%, >99.5% ee). Characterization matched with that of the racemic starting material.

### (S)-1-(2-Methyl-3,4-dihydroquinolin-1(2H)-yl)ethan-1-one (S9.5)

Refer to the synthesis of the racemic form (S2.1).

### (S)-1-(6-Bromo-2-methyl-3,4-dihydroquinolin-1(2H)-yl)ethan-1-one (S9.6)

Refer to the synthesis of the racemic form (S2.2).

### (S)-4-(1-Acetyl-2-methyl-1,2,3,4-tetrahydroquinolin-6-yl)benzaldehyde (S9.7)

A solution of (S)-1-(6-bromo-2-methyl-3,4-dihydroquinolin-1(2H)-yl)ethan-1-one (S9.6) (500 mg, 1.86 mmol), (4-formylphenyl)boronic acid (336 mg, 2.24 mmol), K₂CO₃ (773 mg, 5.59 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-60% EA in PE) to give the title compound as a yellow gel (507 mg, 93%). ¹H NMR (401 MHz, CDCl₃) δ 10.06 (s, 1H), 7.95 (d, J=8.4 Hz, 2H), 7.75 (d, J=8.2 Hz, 2H), 7.49 (dd, J=8.1, 2.0 Hz, 1H), 7.45 (sd, J=1.8 Hz, 1H), 7.27 (br s, 1H), 4.83 (br s, 1H), 2.72 (dt, J=14.8, 5.1 Hz, 1H), 2.67-2.55 (m, 1H), 2.44-2.32 (m, 1H), 2.21 (s, 3H), 1.44 (br s, 1H), 1.17 (d, J=6.4 Hz, 3H). LCMS: (Method A) Rf=6.162 min, (ESI) m/z: 294.2 ([M+H]⁺, 100%).

### (S)-4-(1-Acetyl-2-methyl-1,2,3,4-tetrahydroquinolin-6-yl)benzoic acid (S9.8)

A solution of (S)-1-(6-bromo-2-methyl-3,4-dihydroquinolin-1(2H)-yl)ethan-1-one (S9.6) (1.50 g, 5.59 mmol), (4-carboxylphenyl)boronic acid (1.14 g, 6.87 mmol), K₂CO₃ (3.10 g, 22.43 mmol) in a mixture of DME/H₂O (4:1, 15 mL) was degassed with N₂ for 15 min. Pd(dppf)Cl₂ (409 mg, 0.56 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The aqueous layer was then acidified to pH=1 and extracted with DCM (×3). The organic extracts were combined, washed with brine, dried over MgSO₄ and concentrated in vacuo. The resulting residue was purified using flash column chromatography (0-20% EA in DCM+1% acetic acid) to give the title compound as a white powder (1.51 g, 87%). ¹H NMR resembled that of the racemic form. LCMS: (Method A) Rf=5.722 min, (ESI) m/z: 310.2 ([M+H]⁺, 100%).

### (S)-3-(1-Acetyl-2-methyl-1,2,3,4-tetrahydroquinolin-6-yl)benzoic acid (S9.9)

A solution of (S)-1-(6-bromo-2-methyl-3,4-dihydroquinolin-1(2H)-yl)ethan-1-one (S9.6) (500 mg, 1.86 mmol), (3-carboxylphenyl)boronic acid (372 mg, 2.24 mmol), K₂CO₃ (1.03 g, 7.45 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The murky aqueous layer was filtered through celite. The filtrate was acidified to pH=1 and then extracted with DCM (×3). The organic extracts were combined, washed with brine, dried over MgSO₄ and concentrated in vacuo. The resulting residue was purified using flash column chromatography (0-20% EA in DCM+1% acetic acid) to give the title compound as a white powder (508 mg, 93%). ¹H NMR (401 MHz, CDCl₃) δ 8.36 (t, J=1.7 Hz, 1H), 8.11 (app. dt, 1H), 7.84 (ddd, J=7.8, 1.7, 1.2 Hz, 1H), 7.56 (t, J=7.8 Hz, 1H), 7.49 (dd, J=8.1, 2.0 Hz, 1H), 7.46 (sd, J=1.5 Hz, 1H), 7.24 (br s, 1H), 4.87 (br s, 1H), 2.72 (dt, J=14.7, 4.9 Hz, 1H), 2.67-2.55 (m, 1H), 2.46-2.33 (m, 1H), 2.23 (s, 3H), 1.43 (br s, 1H), 1.18 (d, J=6.5 Hz, 3H). LCMS: (Method C) Rf=3.403 min, (ESI) m/z: 310.1 ([M+H]⁺, 100%).

### (S)-2-(4-(1-Acetyl-2-methyl-1,2,3,4-tetrahydroquinolin-6-yl)phenyl)acetic acid (59.10)

A solution of (S)-1-(6-bromo-2-methyl-3,4-dihydroquinolin-1(2H)-yl)ethan-1-one (S9.6) (500 mg, 1.86 mmol), 4-(carboxymethyl)phenylboronic acid pinacol ester (550 mg, 2.10 mmol), K₂CO₃ (1.03 g, 7.45 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The murky aqueous layer was filtered through celite. The filtrate was acidified to pH=1 and then extracted with DCM (×3). The organic extracts were combined, washed with brine, dried over MgSO₄ and concentrated in vacuo. The resulting residue was purified using flash column chromatography (0-20% EA in DCM+1% acetic acid) to give a thick paste, which was triturated with DCM/PE to give the title compound as a white powder (536 mg, 89%). ¹H NMR (401 MHz, CDCl₃) δ 7.58-7.51 (m, 2H), 7.41 (dd, J=8.1, 1.9 Hz, 1H), 7.39-7.34 (m, 3H), 7.18 (br s, 1H), 4.85 (br s, 1H), 3.70 (s, 2H), 2.68 (dt, J=14.5, 4.7 Hz, 1H), 2.63-2.49 (m, 1H), 2.43-2.31 (m, 1H), 2.20 (s, 3H), 1.36 (br s, 1H), 1.16 (d, J=6.4 Hz, 3H). LCMS: (Method C) Rf=3.383 min, (ESI) m/z: 324.1 ([M+H]⁺, 100%).

### (S)-2-(3-(1-Acetyl-2-methyl-1,2,3,4-tetrahydroquinolin-6-yl)phenyl)acetic acid (59.11)

A solution of (S)-1-(6-bromo-2-methyl-3,4-dihydroquinolin-1(2H)-yl)ethan-1-one (S9.6) (600 mg, 2.24 mmol), 3-(carboxymethyl)phenylboronic acid pinacol ester (644 mg, 2.10 mmol), K₂CO₃ (1.24 g, 8.97 mmol) in a mixture of DME/H₂O (4:1, 5 mL) was degassed with N₂ for 15 min. Pd(dppf)Cl₂ (164 mg, 0.22 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The murky aqueous layer was filtered through celite. The filtrate was acidified to pH=1 and then extracted with DCM (×3). The organic extracts were combined, washed with brine, dried over MgSO₄ and concentrated in vacuo. The resulting residue was purified using flash column chromatography (0-20% EA in DCM+1% acetic acid) to give a thick paste, which was co-evaporated with EA/PE twice to give the title compound as a white powder (614 mg, 85%). ¹H NMR (401 MHz, CDCl₃) δ 7.50 (d, J=8.8 Hz, 2H), 7.45-7.35 (m, 3H), 7.28 (d, J=7.5 Hz, 1H), 7.18 (br s, 1H), 4.85 (br s, 1H), 3.72 (s, 2H), 2.74-2.63 (m, 1H), 2.63-2.49 (m, 1H), 2.43-2.30 (m, 1H), 2.20 (s, 3H), 1.39 (br s, 1H), 1.15 (d, J=6.3 Hz, 3H). LCMS: (Method C) Rf=3.409 min, (ESI) m/z: 324.2 ([M+H]⁺, 100%).

### (S)-1-(6-(4-(Aminomethyl)phenyl)-2-methyl-3,4-dihydroquinolin-1(2H)-yl)ethan-1-one (S9.12)

A solution of (S)-1-(6-bromo-2-methyl-3,4-dihydroquinolin-1(2H)-yl)ethan-1-one (S9.6) (2.0 g, 7.46 mmol), 4-aminomethylphenylboronic acid pinacol ester, hydrochloride (2.26 g, 8.38 mmol), K₂CO₃ (4.13 g, 22.50 mmol) in a mixture of DME/H₂O (4:1, 15 mL) was de gassed with N₂ for 15 min. Pd(dppf)Cl₂ (0.39 g, 0.53 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-5% MeOH in DCM+1% 7N NH₃ in MeOH) to give a thick oil which solidified after a few days and gave the title compound as a pale yellow powder (1.86 g, 85%). ¹H NMR as for the racemic form. LCMS: (Method A) Rf=1.560 min, (ESI) m/z: 278.2 ([(M-NH₂)+H]⁺, 100%), 295.2 ([M+H]⁺, 30%).

### (S)-1-(6-(3-(Aminomethyl)phenyl)-2-methyl-3,4-dihydroquinolin-1(2H)-yl)ethan-1-one (S9.13)

A solution of (S)-1-(6-bromo-2-methyl-3,4-dihydroquinolin-1(2H)-yl)ethan-1-one (S9.6) (500 mg, 1.86 mmol), 3-aminomethylphenylboronic acid, hydrochloride (384 mg, 2.05 mmol), K₂CO₃ (1.03 g, 7.45 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-5% MeOH in DCM+1% 7N NH₃ in MeOH) to give a thick oil which solidified overnight to give the title compound as an off-white solid (390 mg, 71%). ¹H NMR (401 MHz, CDCl₃) δ 7.55 (br s, 1H), 7.50-7.37 (m, 4H), 7.33-7.27 (m, 1H), 7.20 (br s, 1H), 4.84 (br s, 1H), 3.95 (s, 2H), 2.69 (dt, J=14.7, 4.9 Hz, 1H), 2.63-2.50 (m, 1H), 2.37 (ddt, J=10.1, 7.3, 5.1 Hz, 1H), 2.19 (s, 3H), 1.40 (br s, 1H), 1.16 (d, J=6.5 Hz, 3H). LCMS: (Method B) Rf=2.155 min, (ESI) m/z: 295.1 ([M+H]⁺, 100%), 278.2 ([(M-NH₂)+H]⁺, 30%).

### (S)-1-(6-(4-Aminophenyl)-2-methyl-3,4-dihydroquinolin-1(2H)-yl)ethan-1-one (S9.14)

A solution of (S)-1-(6-bromo-2-methyl-3,4-dihydroquinolin-1(2H)-yl)ethan-1-one (S9.6) (500 mg, 1.86 mmol), 4-aminophenylboronic acid pinacol ester (490 mg, 2.24 mmol), K₂CO₃ (775 mg, 5.61 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The organic extracts were combined, washed with brine, dried over MgSO₄ and concentrated in vacuo. The resulting residue was purified using flash column chromatography (0-40% EA in DCM) to give the title compound as a light yellow solid (475 mg, 91%). ¹H NMR (401 MHz, CDCl₃) δ 7.43-7.38 (m, 2H), 7.37 (dd, J=8.2, 1.9 Hz, 1H), 7.33 (s, 1H), 7.13 (br s, 1H), 6.80-6.72 (m, 1H), 4.84 (br s, 1H), 3.75 (br s, 2H), 2.65 (dt, J=14.4, 4.7 Hz, 1H), 2.60-2.48 (m, 1H), 2.36 (ddt, J=9.9, 7.5, 5.0 Hz, 1H), 2.18 (s, 3H), 1.38 (br s, 1H), 1.15 (d, J=6.4 Hz, 3H). LCMS: (Method C) Rf=3.116 min, (ESI) m/z: 281.1 ([M+H]⁺, 100%).

### (S)-1-(6-(3-Aminophenyl)-2-methyl-3,4-dihydroquinolin-1(2H)-yl)ethan-1-one (S9.15)

A solution of (S)-1-(6-bromo-2-methyl-3,4-dihydroquinolin-1(2H)-yl)ethan-1-one (S9.6) (500 mg, 1.86 mmol), 3-aminophenylboronic acid monohydrate (347 mg, 2.24 mmol), K₂CO₃ (775 mg, 5.61 mmol) in a mixture of DME/H₂O (4:1, 5 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The organic extracts were combined, washed with brine, dried over MgSO₄ and concentrated in vacuo. The resulting residue was purified using flash column chromatography (0-40% EA in DCM) to give the title compound as a dark brown solid (514 mg, 92%). ¹H NMR (401 MHz, CDCl₃) δ 7.40 (dd, J=8.2, 1.8 Hz, 1H), 7.36 (s, 1H), 7.22 (t, J=7.8 Hz, 1H), 7.17 (br s, 1H), 6.98 (ddd, J=7.7, 1.6, 1.0 Hz, 1H), 6.90 (st, J=2.0 Hz, 1H), 6.68 (ddd, J=7.9, 2.3, 0.9 Hz, 1H), 4.84 (s, 1H), 3.74 (s, 2H), 2.67 (dt, J=14.7, 4.9 Hz, 1H), 2.62-2.50 (m, 1H), 2.37 (ddt, J=12.7, 7.5, 5.0 Hz, 1H), 2.19 (s, 3H), 1.39 (br s, 1H), 1.16 (d, J=6.5 Hz, 3H). LCMS: (Method C) Rf=3.162 min, (ESI) m/z: 281.1 ([M+H]⁺, 100%).

### (S)-N-(4-(1-Acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010])

A solution of (S)-1-(6-(4-(aminomethyl)phenyl)-2-methyl-3,4-dihydroquinolin-1(2H)-yl)ethan-1-one (S9.12) (1.2 g, 4.08 mmol), lithium(I) 6-bromo-8-morpholinoimidazo[1,2-a]pyrazine-2-carboxylate (S1.3) (1.76 g, 5.28 mmol), HCTU (3.37 g, 8.15 mmol), DIPEA (4.26 mL, 24.45 mmol) in DMF (20 mL) was stirred at r.t o.n. The reaction mixture was then poured into 5% Na₂CO₃ solution, the resulting precipitate was filtered and purified using flash column chromatography (0-3% MeOH in DCM) to give the title compound as a white solid (2.05 g, 83%).

¹H NMR (401 MHz, CDCl₃) δ 8.04 (s, 1H), 7.63 (s, 1H), 7.58-7.49 (m, 3H), 7.45-7.38 (m, 3H), 7.36 (sd, J=1.5 Hz, 1H), 7.19 (br s, 1H), 4.83 (br s, 1H), 4.71 (d, J=6.3 Hz, 2H), 4.29 (br s, 4H), 3.81 (app t, 4H), 2.68 (dt, J=14.7, 4.9 Hz, 1H), 2.62-2.51 (m, 1H), 2.36 (ddt, J=10.2, 7.6, 5.1 Hz, 1H), 2.18 (s, 3H), 1.40 (br s, 1H), 1.15 (d, J=6.4 Hz, 3H). LCMS: (Method A) Rf=6.427 min, (ESI) m/z: 603.2, 605.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-morpholinoimidazo[1,2-a]pyrazine-2-carboxamide [Cpd 010]

A solution of (S)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010]) (1.50 g, 2.49 mmol), 2-aminopyrimidine-5-boronic acid (414 mg, 2.98 mmol), K₂CO₃ (1.03 g, 7.45 mmol) in a mixture of DME/H₂O (4:1, 20 mL) was degassed with N₂ for 15 min. Pd(dppf)Cl₂ (136 mg, 0.19 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-5% MeOH in DCM) to give a brown solid, which was triturated with MeOH to give the title compound as a white powder (1.31 g, 85%). ¹H NMR (401 MHz, CDCl₃) δ 8.81 (s, 2H), 8.14 (s, 1H), 7.83 (s, 1H), 7.61-7.51 (m, 3H), 7.46 (d, J=8.2 Hz, 2H), 7.41 (dd, J=8.2, 1.9 Hz, 1H), 7.37 (s, 1H), 7.20 (br s, 1H), 5.35 (s, 2H), 4.83 (br s, 1H), 4.74 (d, J=6.2 Hz, 2H), 4.33 (app. t, 4H), 3.87 (app. t, 4H), 2.68 (dt, J=14.7, 4.8 Hz, 1H), 2.63-2.52 (m, 1H), 2.42-2.32 (m, 1H), 2.19 (s, 3H), 1.41 (br s, 1H), 1.16 (d, J=6.5 Hz, 3H). LCMS: (Method A) Rf=5.688 min, (ESI) m/z: 618.2 ([M+H]⁺, 100%). HRMS (ESI+) calcd for [C₃₄H₃₅N₉O₃+H]⁺618.2936, found 618.2947.

### (S)-N-(3-(1-Acetyl-2-methyl-1,2,3,4-tetrahydroquinolin-6-yl)benzyl)-6-bromo-8-morpholinoimidazo[1,2-a]pyrazine-2-carboxamide (S17.i[Cpd 011])

A solution of (S)-1-(6-(3-(aminomethyl)phenyl)-2-methyl-3,4-dihydroquinolin-1(2H)-yl)ethan-1-one (S9.13) (100 mg, 0.34 mmol), lithium(I) 6-bromo-8-morpholinoimidazo[1,2-a]pyrazine-2-carboxylate (S1.3) (147 mg, 0.44 mmol), HCTU (281 mg, 0.68 mmol), DIPEA (0.36 mL, 2.06 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with EA and PE to give the title compound as an off-white powder (170 mg, 83%). ¹H NMR (401 MHz, CDCl₃) δ 8.04 (s, 1H), 7.63 (s, 1H), 7.58 (br t, J=1.6 Hz, 1H), 7.54-7.32 (m, 6H), 7.20 (br s, 1H), 4.82 (br s, 1H), 4.75 (d, J=6.3 Hz, 2H), 4.29 (br s, 4H), 3.82 (app. t, 4H), 2.68 (dt, J=14.6, 4.9 Hz, 1H), 2.62-2.51 (m, 1H), 2.36 (ddt, J=10.3, 7.5, 5.1 Hz, 1H), 2.18 (s, 3H), 1.40 (br s, 1H), 1.15 (d, J=6.5 Hz, 3H). LCMS: (Method C) Rf=3.823 min, (ESI) m/z: 603.2, 605.2 ([M+H]⁺, 100%).

### (S)-N-(3-(1-Acetyl-2-methyl-1,2,3,4-tetrahydroquinolin-6-yl)benzyl)-6-(2-aminopyrim-idin-5-yl)-8-morpholinoimidazo[1,2-a]pyrazine-2-carboxamide [Cpd 011]

A solution of (S)-N-(3-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 011]) (110 mg, 0.18 mmol), 2-aminopyrimidine-5-boronic acid (31 mg, 0.22 mmol), K₂CO₃ (76 mg, 0.55 mmol) in a mixture of DME/H₂O (4:1, 5 mL) was degassed with N₂ for 15 min. Pd(dppf)Cl₂ (14 mg, 0.02 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-5% MeOH in DCM) to give a dark yellow solid, which was triturated with MeOH to give the title compound as an off-white powder (91 mg, 81%). ¹H NMR (401 MHz, DMSO-d₆) δ 9.02 (br t, J=6.4 Hz, 1H), 8.78 (s, 2H), 8.44 (s, 1H), 8.27 (s, 1H), 7.63 (s, 1H), 7.54 (d, J=7.8 Hz, 1H), 7.50-7.37 (m, 4H), 7.31 (d, J=7.6 Hz, 1H), 6.88 (s, 2H), 4.71-4.54 (m, 3H), 4.29 (br s, 4H), 3.77 (app. t, 4H), 2.71 (dt, J=15.2, 5.4 Hz, 1H), 2.63-2.52 (m, 1H), 2.36-2.22 (m, 1H), 2.10 (s, 3H), 1.34 (br s, 1H), 1.05 (d, J=6.5 Hz, 3H). LCMS: (Method C) Rf=3.426 min, (ESI) m/z: 618.3 ([M+H]⁺, 100%). HRMS (ESI+) calcd for [C₃₄H₃₅N₉O₃+H]⁺618.2936, found 618.2960.

### (S)-N-(2-(4-(1-Acetyl-2-methyl-1,2,3,4-tetrahydroquinolin-6-yl)benzamido)ethyl)-6-bromo-8-morpholinoimidazo[1,2-a]pyrazine-2-carboxamide (S17.i[Cpd 012])

A solution of N-(2-aminoethyl)-6-bromo-8-morpholinoimidazo[1,2-a]pyrazine-2-carboxamide (S8.1) (110 mg, 0.30 mmol), (S)-4-(1-acetyl-2-methyl-1,2,3,4-tetrahydroquinolin-6-yl)benzoic acid (S9.8) (115 mg, 0.37 mmol), HCTU (250 mg, 0.60 mmol), DIPEA (0.30 mL, 1.70 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as a white powder (168 mg, 86%). Characterisation matches the racemic form.

### (S)-N-(2-(4-(1-Acetyl-2-methyl-1,2,3,4-tetrahydroquinolin-6-yl)benzamido)ethyl)-6-(2-aminopyrimidin-5-yl)-8-morpholinoimidazo[1,2-a]pyrazine-2-carboxamide [Cpd 012]

A solution of (S)-N-(2-(4-(1-acetyl-2-methyl-1,2,3,4-tetrahydroquinolin-6-yl)benzamido)ethyl)-6-bromo-8-morpholinoimidazo[1,2-a]pyrazine-2-carboxamide (S17.i[Cpd 012]) (80 mg, 0.12 mmol), 2-aminopyrimidine-5-boronic acid (26 mg, 0.19 mmol), K₂CO₃ (51 mg, 0.37 mmol) in a mixture of DME/H₂O (4:1, 5 mL) was degassed with N₂ for 15 min. Pd(dppf)Cl₂ (7 mg, 0.01 mmol) was then added and the mixture was further degassed for an other 15 min. The reaction vessel was then evacuated and backfilled with N₂ three times. The reaction mixture was heated to reflux under N₂ for 4 hr. The mixture was then diluted with water and extracted with a mixture of DCM and isopropanol (3:1) (×3). The organic extracts were combined, dried over MgSO₄ and concentrated in vacuo. The resulting residue was purified using flash column chromatography (0-8% MeOH in DCM) to give a dark brown solid, which was triturated with MeOH to give the title compound as an off-white powder (72 mg, 88%). Characterisation matches the racemic form.

### N-(3-Aminopropyl)-6-bromo-8-morpholinoimidazo[1,2-a]pyrazine-2-carboxamide (S13.1)

To a solution of ethyl 6-bromo-8-morpholinoimidazo[1,2-a]pyrazine-2-carboxylate (S1.2) (500 mg, 1.41 mmol) in abs. EtOH (15 mL) was added 1,3-diaminopropane (4 mL). The reaction mixture was heated at 80° C. o.n. The solvent was then concentrated in vacuo. The crude material was added 10% NaOH solution and the resulting precipitate was filtered, washed with water and dried on air to give the title compound as an off-white solid (448 mg, 83%). ¹H NMR (401 MHz, DMSO) δ 8.67 (t, J=5.9 Hz, 1H), 8.24 (s, 1H), 8.14 (s, 1H), 4.24 (br s, 4H), 3.75 (app. t, 4H), 3.37-3.31 (m, 3H), 2.60 (t, J=6.5 Hz, 2H), 1.59 (p, J=6.6 Hz, 2H). LCMS: (Method A) Rf=6.208 min, (ESI) m/z: 383.1, 385.1 ([M+H]⁺, 100%).

### (S)-N-(3-(4-(1-Acetyl-2-methyl-1,2,3,4-tetrahydroquinolin-6-yl)benzamido)propyl)-6-bromo-8-morpholinoimidazo[1,2-a]pyrazine-2-carboxamide (S17.i[Cpd 013])

A solution of N-(3-aminopropyl)-6-bromo-8-morpholinoimidazo[1,2-a]pyrazine-2-carboxamide (100 mg, 0.26 mmol), (S)-4-(1-acetyl-2-methyl-1,2,3,4-tetrahydroquinolin-6-yl)benzoic acid (S9.8) (97 mg, 0.31 mmol), HCTU (216 mg, 0.52 mmol), DIPEA (0.27 mL, 1.55 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as an off-white powder (126 mg, 70%). ¹H NMR (401 MHz, CDCl₃) δ 7.98 (s, 1H), 7.93 (d, J=7.7 Hz, 2H), 7.76 (br t, J=6.0 Hz, 1H), 7.68-7.58 (m, 3H), 7.53 (br t, 1H), 7.44 (d, J=8.2 Hz, 1H), 7.40 (s, 1H), 7.23 (br s, 1H), 4.82 (br s, 1H), 4.34 (br s, 4H), 3.85 (app. t, 4H), 3.72 (s, 1H), 3.66-3.49 (m, 4H), 2.78-2.65 (m, 1H), 2.64-2.50 (m, 1H), 2.44-2.30 (m, 1H), 2.19 (s, 3H), 1.95-1.79 (m, 2H), 1.41 (br s, 1H), 1.15 (d, J=6.1 Hz, 3H). LCMS: (Method A) Rf=5.955 min, (ESI) m/z: 674.2, 676.2 ([M+H]⁺, 100%).

### (S)-N-(3-(4-(1-Acetyl-2-methyl-1,2,3,4-tetrahydroquinolin-6-yl)benzamido)propyl)-6-(2-aminopyrimidin-5-yl)-8-morpholinoimidazo[1,2-a]pyrazine-2-carboxamide [Cpd 013]

A solution of (S)-N-(3-(4-(1-acetyl-2-methyl-1,2,3,4-tetrahydroquinolin-6-yl)benzamido)propyl)-6-bromo-8-morpholinoimidazo[1,2-a]pyrazine-2-carboxamide (S17.i[Cpd 013]) (80 mg, 0.12 mmol), 2-aminopyrimidine-5-boronic acid (20 mg, 0.14 mmol), K₂CO₃ (49 mg, 0.35 mmol) in a mixture of DME/H₂O (4:1, 5 mL) was degassed with N₂ for 15 min. Pd(dppf)Cl₂ (9 mg, 0.01 mmol) was then added and the mixture was further degassed for an other 15 min. The reaction vessel was then evacuated and backfilled with N₂ three times. The reaction mixture was heated to reflux under N₂ for 4 hr. The mixture was then diluted with water and extracted with a mixture of DCM/isopropanol (3:1) (×3). The organic extracts were combined, dried over MgSO₄ and concentrated in vacuo. The resulting residue was purified using flash column chromatography (0-8% MeOH in DCM) to give a brown solid, which was triturated with MeOH to give the title compound as a light beige powder (68 mg, 83%). ¹H NMR (401 MHz, DMSO) δ 8.77 (s, 2H), 8.60-8.51 (m, 2H), 8.43 (s, 1H), 8.23 (s, 1H), 7.90 (d, J=8.5 Hz, 2H), 7.75 (d, J=8.4 Hz, 2H), 7.61-7.51 (m, 2H), 7.43 (br d, J=6.9 Hz, 1H), 6.86 (s, 2H), 4.64 (dd, J=12.9, 6.0 Hz, 1H), 4.30 (br s, 4H), 3.79 (app. t, 4H), 2.72 (dt, J=15.1, 5.5 Hz, 1H), 2.61-2.52 (m, 1H), 2.34-2.22 (m, 1H), 2.11 (s, 3H), 1.79 (p, J=6.4 Hz, 2H), 1.35 (br s, 1H), 1.06 (d, J=6.4 Hz, 3H). LCMS: (Method A) Rf=5.170 min, (ESI) m/z: 689.3 ([M+H]⁺, 100%). HRMS (ESI+) calcd for [C₃₇H₄₀N₁₀O₄+H]⁺689.3307, found 689.3316.

### 6-Bromo-8-morpholinoimidazo[1,2-a]pyrazine-2-carbaldehyde (S13.3) and (6-bromo-8-morpholinoimidazo[1,2-a]pyrazin-2-yl)methanol (S13.4)

A solution of ethyl 6-bromo-8-morpholinoimidazo[1,2-a]pyrazine-2-carboxylate (S1.2) (1.0 g, 2.82 mmol) in dry DCM (40 mL) was evacuated and backfilled with N₂ three times. The solution was then cooled to −78° C. and DIBAL (1 M in toluene) (8.4 mL, 8.4 mmol) was added dropwise. The reaction mixture was stirred at this temperature for 40 min. The reaction was then quenched by dropwise addition of cold MeOH (25 mL) at −78° C. and stirred for 15 min. The cold reaction mixture was poured into sat. bicarb solution and the heterogenous mixture was stirred vigorously until reached r.t. The heterogenous mixture was then filtered through celite. The filtrate was extracted with DCM (×2) and celite pad was washed with a mixture of DCM/MeOH (3:1). The organics were combined, dried over MgSO₄ and concentrated in vacuo. The crude material was then purified using flash column chromatography (0-5% MeOH in DCM) to isolate the aldehyde (460 mg, 52%) as a pale yellow solid and the alcohol as an off-white solid (220 mg, 25%).

### 6-Bromo-8-morpholinoimidazo[1,2-a]pyrazine-2-carbaldehyde (S13.3)

¹H NMR (401 MHz, CDCl₃) δ 10.04 (s, 3H), 7.98 (s, 3H), 7.61 (s, 3H), 4.40 (br s, 12H), 3.86 (app. t, 13H). LCMS: (Method A) Rf=4.517 min, (ESI) m/z: 310.9, 312.9 ([M+H]⁺, 100%).

### (6-Bromo-8-morpholinoimidazo[1,2-a]pyrazin-2-yl)methanol (S13.4)

¹H NMR (401 MHz, DMSO) δ 8.16 (s, 1H), 7.77 (s, 1H), 5.28 (t, J=5.7 Hz, 1H), 4.56 (d, J=6.1 Hz, 2H), 4.18 (app. t, 4H), 3.72 (app. t, 4H). LCMS: (Method A) Rf=4.029 min, (ESI) m/z: 314.0, 316.0 ([M+H]⁺, 100%).

### tert-Butyl 4-((6-bromo-8-morpholinoimidazo[1,2-a]pyrazin-2-yl)methyl)piperazine-1-carboxylate (513.5)

To a solution of 6-bromo-8-morpholinoimidazo[1,2-a]pyrazine-2-carbaldehyde (S13.3) (400 mg, 1.29 mmol) in dry DCE (15 mL) was added Boc-piperazine (479 mg, 2.57 mmol), sodium triacetoxyborohydride (817 mg, 3.85 mmol) and 3A MS. The reaction vessel was then evacuated and backfilled with N₂ three times. The reaction mixture was heated to reflux under N₂ for 2 hr. The reaction mixture was then filtered through celite and washed with a mixture of DCM/MeOH (9:1). The solvent was then concentrated in vacuo, the crude material was used directly in the next step without further purification. LCMS: (Method A) Rf=3.923 min, (ESI) m/z: 480.9, 482.9 ([M+H]⁺, 100%).

### 5-(8-Morpholino-2-(piperazin-1-ylmethyl)imidazo[1,2-a]pyrazin-6-yl)pyrimidin-2-amine (di-TFA salt) (S13.6)

Step 1: A solution of crude tert-butyl 4-((6-bromo-8-morpholinoimidazo[1,2-a]pyrazin-2-yl)methyl)piperazine-1-carboxylate (S13.5) from the last step (1.29 mmol), 2-aminopyrimi-dine-5-boronic acid (268 mg, 1.93 mmol), K₂CO₃ (533 mg, 3.86 mmol) in a mixture of DME/H₂O (4:1, 10 mL) was degassed with N₂ for 15 min. Pd(dppf)Cl₂ (94 mg, 0.13 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with a mixture of DCM/isopropanol (3:1) (×3). The organic extracts were combined, dried over MgSO₄ and concentrated in vacuo. The resulting residue was triturated with MeOH to give tert-Butyl 4-((6-(2-aminopyrimidin-5-yl)-8-morpholinoimidazo[1,2-a]pyrazin-2-yl)methyl)piperazine-1-carboxylate as a grey powder (457 mg, 72%) LCMS: (Method A) Rf=3.488 min, (ESI) m/z: 496.1 ([M+H]⁺, 100%). ¹H NMR (401 MHz, DMSO) δ 8.76 (s, 2H), 8.40 (s, 1H), 7.75 (s, 1H), 6.82 (s, 2H), 4.22 (app .t, 4H), 3.76 (app .t, 4H), 3.61 (s, 2H), 3.31 (br s, 4H, partially overlapped with the water peak), 2.40 (app .t, 4H), 1.38 (s, 9H). Step 2: A solution of tert-butyl 4-((6-(2-aminopyrimidin-5-yl)-8-morpholinoimidazo[1,2-a]pyrazin-2-yl)methyl)piperazine-1-carboxylate (430 mg, 0.87 mmol) in a mixture of DCM/TFA (1:1, 6 mL) was stirred at r.t for 1 hr. The solvent was then concentrated in vacuo. The resulting residue was then triturated with MeOH and diethyl ether to give the title compound as a brown powder (508 mg, 94%). ¹H NMR (401 MHz, MeOD) δ 8.91 (s, 2H), 8.32 (s, 1H), 7.86 (s, 1H), 4.29 (app. t, 4H), 4.06 (s, 2H), 3.85 (app. t, 4H), 3.36 (app. t, 4H), 3.09 (app. t, 4H). LCMS: (Method A) Rf=3.033 min, (ESI) m/z: 396.1 ([M+H]⁺, 100%).

### (S)-1-(6-(4-(4-((6-(2-Aminopyrimidin-5-yl)-8-morpholinoimidazo[1,2-a]pyrazin-2-yl)methyl)piperazine-1-carbonyl)phenyl)-2-methyl-3,4-dihydroquinolin-1(2H)-yl)ethan-1-one [Cpd 014]

A solution of (S)-4-(1-acetyl-2-methyl-1,2,3,4-tetrahydroquinolin-6-yl)benzoic acid (S9.8) (74 mg, 0.24 mmol), HCTU (133 mg, 0.32 mmol), DIPEA (0.22 mL, 1.26 mmol) in DMF (5 mL) was stirred at r.t for 15 min. To this solution was then added 5-(8-morpholino-2-(piperazin-1-ylmethyl)imidazo[1,2-a]pyrazin-6-yl)pyrimidin-2-amine (di-TFA salt) (513.6) (100 mg, 0.16 mmol) and reaction mixture was stirred at r.t o.n. The reaction mixture was then poured into 5% Na₂CO₃ solution, the resulting precipitate was filtered and purified using flash column chromatography (0-5% MeOH in DCM) to give a light beige powder (90 mg, 82%).

¹H NMR (401 MHz, CDCl₃) δ 8.79 (br s, 2H), 7.77 (s, 1H), 7.61 (d, J=8.4 Hz, 2H), 7.51-7.45 (m, 3H), 7.42 (dd, J=8.2, 2.0 Hz, 1H), 7.38 (sd, J=1.4 Hz, 1H), 7.23 (br s, 1H), 5.17 (s, 2H), 4.83 (s, 1H), 4.34 (app. t, 4H), 4.00-3.79 (m, 6H), 3.75 (s, 2H), 3.54 (br s, 2H), 2.76-2.50 (m, 6H), 2.37 (ddt, J=10.5, 7.7, 5.2 Hz, 1H), 2.20 (s, 3H), 1.42 (br s, 1H), 1.16 (d, J=6.5 Hz, 3H). LCMS: (Method A) Rf=3.794 min, (ESI) m/z: 687.0 ([M+H]⁺, 100%). HRMS (ESI⁺) calcd for [C₃₈H₄₂N₁₀O₃+H]⁺687.3514, found 687.3536.

### (S)-1-(6-(4-((4-((6-(2-Aminopyrimidin-5-yl)-8-morpholinoimidazo[1,2-a]pyrazin-2-yl)methyl)piperazin-1-yl)methyl)phenyl)-2-methyl-3,4-dihydroquinolin-1(2H)-yl)ethan-1-one [Cpd 015]

A solution of 5-(8-morpholino-2-(piperazin-1-ylmethyl)imidazo[1,2-a]pyrazin-6-yl)pyrimidin-2-amine (di-TFA salt) (S13.6) (100 mg, 0.16 mmol), (S)-4-(1-acetyl-2-methyl-1,2,3,4-tetrahydroquinolin-6-yl)benzaldehyde (S9.7) (71 mg, 0.24 mmol) and glacial acetic acid (0.18 mL, 3.08 mmol) in dry DCE (15 mL) was stirred at r.t for 15 min. To this mixture was then added sodium triacetoxyborohydride (102 mg, 0.48 mmol). The reaction vessel was then evacuated and backfilled with N₂ three times. The reaction mixture was heated to reflux under N₂ o.n. Another portion of sodium triacetoxyborohydride was added and the reaction mixture was stirred for another 12 hr. The reaction mixture was then diluted with DCM, washed with 10% NaOH solution, dried over MgSO₄ and the solvent concentrated in vacuo. The residue was then purified using flash column chromatography (0-7% MeOH (1% 7N NH₃ in MeOH) in DCM) to give an oil, which was triturated with DCM and PE to give the title compound as a pale yellow powder (36 mg, 34%). ¹H NMR (401 MHz, CDCl₃) δ 8.78 (s, 2H), 7.76 (s, 1H), 7.53 (d, J=8.2 Hz, 2H), 7.46-7.31 (m, 4H), 7.19 (br s, 1H), 5.15 (s, 2H), 4.84 (br s, 1H), 4.40-4.27 (m, 4H), 3.91-3.84 (m, 4H), 3.77 (br s, 2H), 3.59 (br s, 2H), 2.93-2.45 (m, 10H), 2.37 (ddt, J=10.4, 7.7, 5.1 Hz, 1H), 2.19 (s, 3H), 1.40 (br s, 1H), 1.16 (d, J=6.5 Hz, 3H). LCMS: (Method A) Rf=3.865 min, (ESI) m/z: 673.1 ([M+H]⁺, 100%). HRMS (ESI⁺) calcd for [C₃₈H₄₄N₁₀O₂+H]⁺673.3721, found 673.3717.

### 4-(6-Bromo-2-(bromomethyl)imidazo[1,2-a]pyrazin-8-yl)morpholine (S13.7)

A solution of (6-bromo-8-morpholinoimidazo[1,2-a]pyrazin-2-yl)methanol (S13.4) (840 mg, 2.68 mmol) in dry toluene was cooled to 0° C. PBr₃ (0.28 mL, 2.94 mmol) was added dropwise to the cold solution and gave a thick suspension. The reaction vessel was then evacuated and backfilled with N₂ three times. The suspension was then heated to reflux under N₂ for 1 hr. The reaction mixture was then cooled to 0° C. and quenched by water. The heterogenous mixture was then extracted with EA (×2). The organic extracts were combined, dried over MgSO₄ and concentrated in vacuo. The residue was then triturated with cold diethyl ether to give the title compound as an off-white powder. (815 mg, 81%). ¹H NMR (401 MHz, DMSO) δ 8.14 (s, 1H), 7.97 (s, 1H), 4.76 (s, 2H), 4.18 (br s, 4H), 3.73 (app. t, 4H). LCMS: (Method C) Rf=3.789 min, (ESI) m/z: 374.9 ([M+H]⁺, 50%), 376.9 ([M+H]⁺, 100%), 378.9 ([M+H]⁺, 50%).

### (6-Bromo-8-morpholinoimidazo[1,2-a]pyrazin-2-yl)methanamine (S13.8)

To a sealed tube was charged with a suspension of 4-(6-bromo-2-(bromomethyl)imidazo[1,2-a]pyrazin-8-yl)morpholine (S13.7) (340 mg, 0.90 mmol) in a mixture of MeOH and 30% NH₃ (1:1, 20 mL). The reaction mixture was then heated at 100° C. for 2 hr. The MeOH was then concentrated in vacuo. The remaining aqueous residue was acidified to pH=1 with 1N HCl and extracted with DCM (×2). The aqueous layer was then basified to pH=14 with 10% NaOH solution and exacted with DCM (×3). The organic extracts were combined, dried over MgSO₄ and concentrated in vacuo to give the title compound as a white powder (257 mg, 89%). LCMS: (Method C) Rf=2.873 min, (ESI) m/z: 312.0, 314.0 ([M+H]⁺, 100%). ¹H NMR (401 MHz, CDCl₃) δ 7.56 (s, 1H), 7.34 (s, 1H), 4.32 (app. t, 4H), 3.97 (s, 2H), 3.84 (app. t, 4H).

### (S)-4-(1-Acetyl-2-methyl-1,2,3,4-tetrahydroquinolin-6-yl)-N-((6-bromo-8-morpholinoimidazo[1,2-a]pyrazin-2-yl)methyl)benzamide (S17.i[Cpd 016])

To a solution of (6-bromo-8-morpholinoimidazo[1,2-a]pyrazin-2-yl)methanamine (S13.8) (70 mg, 0.22 mmol) in DMF (5 mL) was added (S)-4-(1-acetyl-2-methyl-1,2,3,4-tetra-hydroquinolin-6-yl)benzoic acid (S9.8) (83 mg, 0.27 mmol), EDC.HCl (86 mg, 0.45 mmol), HOBT (46 mg, 0.34 mmol) and DIPEA (0.23 mL, 1.35 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was co-evaporated with DCM and PE to give the title compound as a white powder (112 mg, 83%). ¹H NMR (401 MHz, CDCl₃) δ 7.87 (d, J=8.4 Hz, 2H), 7.64 (d, J=8.4 Hz, 2H), 7.56 (s, 1H), 7.48 (s, 1H), 7.44 (dd, J=8.2, 1.9 Hz, 1H), 7.41 (br s, 1H), 7.23 (br s, 1H), 6.96 (br t, J=5.3 Hz, 1H), 4.82 (br s, 1H), 4.74 (d, J=5.4 Hz, 2H), 4.30 (app. t, 4H), 3.83 (app. t, 4H), 2.70 (dt, J=14.7, 5.0 Hz, 1H), 2.64-2.52 (m, 1H), 2.42-2.31 (m, 1H), 2.18 (br s, 1H), 1.39 (s, 2H), 1.21 (s, 2H), 1.16 (d, J=6.5 Hz, 3H). LCMS: (Method C) Rf=3.698 min, (ESI) m/z: 603.2, 605.2 ([M+H]⁺, 100%).

### (S)-4-(1-Acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide [Cpd 016]

A solution of (S)-4-(1-acetyl-2-methyl-1,2,3,4-tetrahydroquinolin-6-yl)-N-((6-bromo-8-morpholinoimidazo[1,2-a]pyrazin-2-yl)methyl)benzamide (S17.i[Cpd 016]) (70 mg, 0.12 mmol), 2-aminopyrimidine-5-boronic acid (20 mg, 0.14 mmol), K₂CO₃ (48 mg, 0.35 mmol) in a mixture of DME/H₂O (4:1, 5 mL) was degassed with N₂ for 15 min. Pd(dppf)Cl₂ (9 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-6% MeOH in DCM) to give a yellow solid, which was triturated with cold MeOH to give the title compound as an off-white powder (47 mg, 66%). ¹H NMR (401 MHz, CDCl₃) δ 8.78 (s, 2H), 7.88 (d, J=8.4 Hz, 2H), 7.77 (s, 1H), 7.66 (d, J=8.5 Hz, 2H), 7.57 (s, 1H), 7.46 (dd, J=8.2, 2.0 Hz, 1H), 7.42 (br s, 1H), 7.21 (br s, 1H), 6.88 (br t, J=5.3 Hz, 1H), 5.15 (s, 2H), 4.82 (br s, 1H), 4.78 (d, J=5.4 Hz, 2H), 4.35 (app. t, 4H), 3.89 (app. t, 4H), 2.71 (dt, J=10.1, 4.8 Hz, 1H), 2.60 (ddd, J=14.1, 10.1, 4.3 Hz, 1H), 2.43-2.31 (m, 1H), 2.20 (s, 3H), 1.42 (br s, 1H), 1.16 (d, J=6.5 Hz, 3H). LCMS: (Method C) Rf=3.277 min, (ESI) m/z: 618.3 ([M+H]⁺, 100%). HRMS (ESI⁺) calcd for [C₃₄H₃₅N₉O₃+H]⁺618.2936, found 618.2957.

### (S)-3-(1-Acetyl-2-methyl-1,2,3,4-tetrahydroquinolin-6-yl)-N-((6-bromo-8-morpholinoimidazo[1,2-a]pyrazin-2-yl)methyl)benzamide (S17.i[Cpd 017])

To a solution of (6-bromo-8-morpholinoimidazo[1,2-a]pyrazin-2-yl)methanamine (S13.8) (60 mg, 0.19 mmol) in DMF (5 mL) was added (S)-3-(1-acetyl-2-methyl-1,2,3,4-tetra-hydroquinolin-6-yl)benzoic acid (S9.9) (72 mg, 0.23 mmol), EDC.HCl (74 mg, 0.39 mmol), HOBT (40 mg, 0.30 mmol) and DIPEA (0.20 mL, 1.15 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was co-evaporated with DCM and PE to give the title compound as a white powder (97 mg, 84%). ¹H NMR (401 MHz, CDCl₃) δ 8.05 (st, J=1.6 Hz, 1H), 7.77-7.69 (m, 2H), 7.57 (s, 1H), 7.54-7.40 (m, 4H), 7.23 (br s, 1H), 7.04 (br s, 1H), 4.83 (br s, 1H), 4.76 (d, J=5.5 Hz, 2H), 4.39-4.17 (m, 4H), 3.89-3.74 (m, 4H), 2.69 (dt, J=14.7, 5.0 Hz, 1H), 2.64-2.51 (m, 1H), 2.43-2.29 (m, 1H), 2.19 (s, 3H), 1.42 (br s, 2H), 1.16 (d, J=6.5 Hz, 3H). LCMS: (Method C) Rf=3.730 min, (ESI) m/z: 603.2, 605.2 ([M+H]⁺, 100%).

### (S)-3-(1-Acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide [Cpd 017]

A solution of (S)-3-(1-acetyl-2-methyl-1,2,3,4-tetrahydroquinolin-6-yl)-N-((6-bromo-8-morpholinoimidazo[1,2-a]pyrazin-2-yl)methyl)benzamide (S17.i[Cpd 017]) (70 mg, 0.12 mmol), 2-aminopyrimidine-5-boronic acid (20 mg, 0.14 mmol), K₂CO₃ (48 mg, 0.35 mmol) in a mixture of DME/H₂O (4:1, 5 mL) was degassed with N₂ for 15 min. Pd(dppf)Cl₂ (9 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as an off-white powder (47 mg, 66%). ¹H NMR (401 MHz, CDCl₃) δ 8.78 (s, 2H), 8.05 (st, J=1.6 Hz, 1H), 7.76 (s, 1H), 7.76-7.70 (m, 2H), 7.57 (s, 1H), 7.51 (t, J=7.7 Hz, 1H), 7.45 (dd, J=8.1, 2.0 Hz, 1H), 7.42 (br s, 1H), 7.23 (br s, 1H), 6.95 (br t, J=5.3 Hz, 1H), 5.17 (s, 2H), 4.82 (br s, 1H), 4.78 (d, J=5.4 Hz, 2H), 4.33 (app. t, 4H), 3.87 (app. t, 4H), 2.69 (dt, J=15.2, 5.0 Hz, 1H), 2.64-2.53 (m, 1H), 2.43-2.32 (m, 1H), 2.19 (s, 3H), 1.41 (br s, 1H), 1.16 (d, J=6.5 Hz, 3H). LCMS: (Method C) Rf=3.305 min, (ESI) m/z: 618.3 ([M+H]⁺, 100%). HRMS (ESI⁺) calcd for [C₃₄H₃₅N₉O₃+H]⁺618.2936, found 618.2945.

### (S)-2-(4-(1-Acetyl-2-methyl-1,2,3,4-tetrahydroquinolin-6-yl)phenyl)-N-((6-bromo-8-morpholinoimidazo[1,2-a]pyrazin-2-yl)methyl)acetamide (S17.i[Cpd 018])

To a solution of (6-bromo-8-morpholinoimidazo[1,2-a]pyrazin-2-yl)methanamine (S13.8) (60 mg, 0.19 mmol) in DMF (5 mL) was (S)-2-(4-(1-acetyl-2-methyl-1,2,3,4-tetrahy-droquinolin-6-yl)phenyl)acetic acid (S9.10) (75 mg, 0.23 mmol), EDC.HCl (74 mg, 0.39 mmol), HOBT (39 mg, 0.30 mmol) and DIPEA (0.20 mL, 1.15 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was triturated with EA and PE to give the title compound as a white powder (99 mg, 83%). ¹H NMR (401 MHz, CDCl₃) δ 7.56 (d, J=8.2 Hz, 2H), 7.52 (s, 1H), 7.42 (dd, J=8.2, 1.9 Hz, 1H), 7.38 (br s, 1H), 7.33 (d, J=8.8 Hz, 3H), 7.22 (br s, 1H), 6.18 (br s, 1H), 4.83 (br s, 1H), 4.51 (d, J=5.5 Hz, 2H), 4.21 (app. t, 4H), 3.77 (app. t, 4H), 3.65 (s, 2H), 2.69 (dt, J=14.6, 4.8 Hz, 1H), 2.64-2.52 (m, 1H), 2.43-2.31 (m, 1H), 2.19 (s, 3H), 1.42 (br s, 1H), 1.16 (d, J=6.5 Hz, 3H). LCMS: (Method C) Rf=3.652 min, (ESI) m/z: 617.2, 619.2 ([M+H]⁺, 100%).

### (S)-2-(4-(1-Acetyl-2-methyl-1,2,3,4-tetrahydroquinolin-6-yl)phenyl)-N-((6-(2-aminopy-rimidin-5-yl)-8-morpholinoimidazo[1,2-a]pyrazin-2-yl)methyl)acetamide [Cpd 018]

A solution of (S)-2-(4-(1-acetyl-2-methyl-1,2,3,4-tetrahydroquinolin-6-yl)phenyl)-N-((6-bromo-8-morpholinoimidazo[1,2-a]pyrazin-2-yl)methyl)acetamide (S17.i[Cpd 018]) (70 mg, 0.11 mmol), 2-aminopyrimidine-5-boronic acid (19 mg, 0.14 mmol), K₂CO₃ (47 mg, 0.35 mmol) in a mixture of DME/H₂O (4:1, 5 mL) was degassed with N₂ for 15 min. Pd(dppf)Cl₂ (9 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as a white powder (57 mg, 80%). ¹H NMR (401 MHz, CDCl₃) δ 8.75 (s, 2H), 7.71 (s, 1H), 7.57 (d, J=8.2 Hz, 2H), 7.45-7.32 (m, 5H), 7.21 (br s, 1H), 6.17 (br t, J=5.2 Hz, 1H), 5.19 (s, 2H), 4.84 (br s, 1H), 4.53 (d, J=5.4 Hz, 2H), 4.25 (app. t, 4H), 3.81 (app. t, 4H), 3.67 (s, 2H), 2.69 (dt, J=14.5, 4.7 Hz, 1H), 2.63-2.52 (m, 1H), 2.42-2.31 (m, 1H), 2.19 (s, 3H), 1.40 (br s, 1H), 1.16 (d, J=6.5 Hz, 3H). LCMS: (Method C) Rf=3.270 min, (ESI) m/z: 632.3 ([M+H]⁺, 100%). HRMS (ESI⁺) calcd for [C₃₅H₃₇N₉O₃+H]⁺632.3092, found 632.3103.

### (S)-2-(3-(1-Acetyl-2-methyl-1,2,3,4-tetrahydroquinolin-6-yl)phenyl)-N-((6-bromo-8-morpholinoimidazo[1,2-a]pyrazin-2-yl)methyl)acetamide (S17.i[Cpd 019])

To a solution of (6-bromo-8-morpholinoimidazo[1,2-a]pyrazin-2-yl)methanamine (S13.8) (60 mg, 0.19 mmol) in DMF (5 mL) was (S)-2-(3-(1-acetyl-2-methyl-1,2,3,4-tetrahy-droquinolin-6-yl)phenyl)acetic acid (S9.11) (75 mg, 0.23 mmol), EDC.HCl (74 mg, 0.39 mmol), HOBT (39 mg, 0.30 mmol) and DIPEA (0.20 mL, 1.15 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as a white powder (101 mg, 85%). ¹H NMR (401 MHz, CDCl₃) δ 7.57-7.46 (m, 3H), 7.45-7.34 (m, 3H), 7.31 (s, 1H), 7.25 (d, J=8.0 Hz, 1H), 7.21 (br s, 1H), 6.21 (br s, 1H), 4.83 (br s, 1H), 4.51 (d, J=5.4 Hz, 2H), 4.18 (app. t, 4H), 3.75 (app. t, 4H), 3.68 (s, 2H), 2.66 (dt, J=14.5, 4.9 Hz, 1H), 2.62-2.50 (m, 1H), 2.44-2.30 (m, 1H), 2.19 (s, 3H), 1.41 (br s, 1H), 1.16 (d, J=6.5 Hz, 3H). LCMS: (Method C) Rf=3.660 min, (ESI) m/z: 617.2, 619.2 ([M+H]⁺, 100%).

### (S)-2-(3-(1-Acetyl-2-methyl-1,2,3,4-tetrahydroquinolin-6-yl)phenyl)-N-((6-(2-aminopy-rimidin-5-yl)-8-morpholinoimidazo[1,2-a]pyrazin-2-yl)methyl)acetamide [Cpd 019]

A solution of (S)-2-(3-(1-acetyl-2-methyl-1,2,3,4-tetrahydroquinolin-6-yl)phenyl)-N-((6-bromo-8-morpholinoimidazo[1,2-a]pyrazin-2-yl)methyl)acetamide ((S17.i[Cpd 019]) (70 mg, 0.11 mmol), 2-aminopyrimidine-5-boronic acid (19 mg, 0.14 mmol), K₂CO₃ (47 mg, 0.35 mmol) in a mixture of DME/H₂O (4:1, 5 mL) was degassed with N₂ for 15 min. Pd(dppf)Cl₂ (9 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as an off-white powder (49 mg, 69%). ¹H NMR (401 MHz, CDCl₃) δ 8.75 (s, 2H), 7.70 (s, 1H), 7.56-7.49 (m, 2H), 7.46-7.35 (m, 4H), 7.27-7.24 (m, 1H), 7.20 (br s, 1H), 6.19 (br t, J=5.1 Hz, 1H), 5.17 (s, 2H), 4.82 (br s, 1H), 4.53 (d, J=5.4 Hz, 2H), 4.22 (app. t, 4H), 3.79 (app. t, 4H), 3.70 (s, 2H), 2.66 (dt, J=14.7, 4.8 Hz, 1H), 2.61-2.51 (m, 1H), 2.42-2.30 (m, 1H), 2.18 (s, 3H), 1.40 (br s, 1H), 1.15 (d, J=6.5 Hz, 3H). LCMS: (Method C) Rf=3.274 min, (ESI) m/z: 632.3 ([M+H]⁺, 100%). HRMS (ESI⁺) calcd for [C₃₅H₃₇N₉O₃+H]⁺632.3092, found 632.3104.

### 5-Bromo-3-morpholinopyrazin-2-amine (514.1)

To a sealed tube was charged with a solution of 3,5-dibromopyrazin-2-amine (1.28 g, 5.06 mmol) in morpholine (1.3 mL, 15.07 mmol). The reaction mixture was heated at 120° C. o.n. The suspension was dissolved in DCM and washed with sat. bicarb solution (×2), brine, dried over MgSO₄. The solvent was concentrated in vacuo and the residue was triturated with cold diethyl ether to give the title compound as a pale yellow powder (1.13 g, 86%). ¹H NMR (401 MHz, CDCl₃) δ 7.78 (s, 1H), 4.61 (br s, 2H), 3.84 (app. t, 4H), 3.21 (app. t, 4H). LCMS: (Method A) Rf=4.066 min, (ESI) m/z: 258.9, 260.9 ([M+H]⁺, 100%).

### Ethyl 6-bromo-3-methyl-8-morpholinoimidazo[1,2-a]pyrazine-2-carboxylate (S14.2)

A solution of 5-bromo-3-morpholinopyrazin-2-amine (S14.1) (880 mg, 3.40 mmol) and ethyl 3-bromo-2-oxobutanoate (770 mg, 3.68 mmol) in dry DME (3 mL) was heated at 100° C. for 20 hr. The reaction mixture was diluted with DCM and washed with sat. bicarb solution (×2), dried over MgSO₄. The solvent was concentrated in vacuo and the residue purified using flash column chromatography (0-40% EA in PE) to give the title compound as a white powder (801 mg, 64%). ¹H NMR (401 MHz, CDCl₃) δ 7.37 (s, 1H), 4.42 (q, J=7.1 Hz, 2H), 4.37 (br s, 4H), 3.88-3.79 (app. t, 4H), 2.68 (s, 3H), 1.42 (t, J=7.1 Hz, 3H). LCMS: (Method A) Rf=5.072 min, (ESI) m/z: 368.9, 370.9 ([M+H]⁺, 100%).

### Lithium(I) 6-bromo-3-methyl-8-morpholinoimidazo[1,2-a]pyrazine-2-carboxylate (S14.3)

To a solution of ethyl 6-bromo-3-methyl-8-morpholinoimidazo[1,2-a]pyrazine-2-carboxylate (S14.2) (800 mg, 2.17 mmol) in a mixture of THE and water (4:1, 15 mL) was added lithium hydroxide monohydrate (92 mg, 2.19 mmol). The reaction mixture was heated to reflux for 3 hr before cooling to r.t. The organic solvent was concentrated and to the residual aqueous layer was added acetone. The resulting precipitate was collected via filtration and dried under vacuum to give the title compound as a white powder (709 mg, 94%). ¹H NMR (401 MHz, D₂O) δ 7.15 (s, 1H), 3.94 (br s, 4H), 3.75 (br s, 4H), 2.36 (s, 3H). LCMS: (Method A) Rf=4.359 min, (ESI) m/z: 340.9, 342.9 ([M+H]⁺, 100%).

### (S)-N-(4-(1-Acetyl-2-methyl-1,2,3,4-tetrahydroquinolin-6-yl)benzyl)-6-bromo-3-methyl-8-morpholinoimidazo[1,2-a]pyrazine-2-carboxamide (S17.i[Cpd 020])

A solution of (S)-1-(6-(4-(aminomethyl)phenyl)-2-methyl-3,4-dihydroquinolin-1(2H)-yl)ethan-1-one (S9.12) (110 mg, 0.37 mmol), lithium(I) 6-bromo-3-methyl-8-morpholinoimidazo[1,2-a]pyrazine-2-carboxylate (S14.3) (165 mg, 0.48 mmol), HCTU (400 mg, 0.97 mmol), DIPEA (0.50 mL, 2.90 mmol) in DMF (5 mL) was stirred at r.t o.n. Another portion of HCTU (200 mg, 0.48 mmol) was added and the reaction mixture was allowed to stirred at r.t for another 4 hr. The reaction mixture was then poured into 5% Na₂CO₃ solution, the resulting precipitate was filtered and purified using flash column chromatography (0-3% MeOH in DCM) to give an oil, which was triturated with EA and PE to give the title compound as an off-white powder (210 mg, 91%). ¹H NMR (401 MHz, DMSO) δ 8.98 (t, J=6.4 Hz, 1H), 7.95 (s, 1H), 7.63 (d, J=8.3 Hz, 2H), 7.52-7.44 (m, 2H), 7.39 (br s, 1H, d, J=8.3 Hz, 2H), 4.68-4.58 (m, 1H), 4.53 (d, J=6.3 Hz, 2H), 4.25 (br s, 4H), 3.78-3.72 (m, 4H), 2.86-2.64 (m, 1H), 2.69 (s, 3H), 2.58-2.53 (m, 1H), 2.36-2.21 (m, 1H), 2.10 (s, 3H), 1.34 (br s, 1H), 1.05 (d, J=6.5 Hz, 3H). LCMS: (Method A) Rf=5.444 min, (ESI) m/z: 617.0, 619.0 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-3-methyl-8-morpholinoimidazo[1,2-a]pyrazine-2-carboxamide [Cpd 020]

A solution of (S)-N-(4-(1-acetyl-2-methyl-1,2,3,4-tetrahydroquinolin-6-yl)benzyl)-6-bromo-3-methyl-8-morpholinoimidazo[1,2-a]pyrazine-2-carboxamide (S17.i[Cpd 020]) (100 mg, 0.16 mmol), 2-aminopyrimidine-5-boronic acid (27 mg, 0.19 mmol), K₂CO₃ (67 mg, 0.48 mmol) in a mixture of DME/H₂O (4:1, 5 mL) was degassed with N₂ for 15 min. Pd(dppf)Cl₂ (12 mg, 0.02 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as an off-white powder (95 mg, 84%). ¹H NMR (401 MHz, CDCl₃) δ 8.83 (s, 2H), 7.65 (br t, J=6.2 Hz, 1H), 7.59-7.55 (m, 3H), 7.46 (d, J=8.3 Hz, 2H), 7.42 (dd, J=8.2, 1.9 Hz, 1H), 7.37 (sd, J=1.5 Hz, 1H), 7.20 (br s, 1H), 5.20 (s, 2H), 4.84 (br s, 1H), 4.73 (d, J=6.3 Hz, 2H), 4.34 (app. t, 4H), 3.87 (app. t, 4H), 2.87 (s, 3H), 2.68 (dt, J=14.9, 4.8 Hz, 1H), 2.63-2.53 (m, 1H), 2.37 (ddt, J=10.2, 7.3, 5.1 Hz, 1H), 2.19 (s, 3H), 1.41 (br s, 1H), 1.16 (d, J=6.5 Hz, 3H). LCMS: (Method A) Rf=4.673 min, (ESI) m/z: 632.0 ([M+H]⁺, 100%). HRMS (ESI⁺) calcd for [C₃₅H₃₇N₉O₃+H]⁺632.3092, found 632.3115.

### N-(2-Aminoethyl)-6-bromo-3-methyl-8-morpholinoimidazo[1,2-a]pyrazine-2-carboxamide (514.4)

To a solution of ethyl 6-bromo-3-methyl-8-morpholinoimidazo[1,2-a]pyrazine-2-carboxylate (S14.2) (210 mg, 0.57 mmol) in abs. EtOH (5 mL) was added ethylenediamine (5 mL). The reaction mixture was heated to reflux for 4 hr. The EtOH was removed in vacuo. The residue was diluted with DCM and washed with 10% NaOH solution (×2). The organic was dried over MgSO₄, concentrated in vacuo. The resulting residue was triturated with DCM and PE to give the title compound as a pale yellow powder (214 mg, 98%). ¹H NMR (401 MHz, DMSO) δ 8.30 (br t, J=6.0 Hz, 4H), 7.92 (br s, 4H), 4.23 (s, 17H), 3.74 (app. t, 18H), 3.27 (dd, J=12.8, 6.5 Hz, 15H), 2.69 (t, J=6.6 Hz, 8H), 2.66 (s, 13H). LCMS: (Method B) Rf=3.605 min, (ESI) m/z: 382.9, 384.9 ([M+H]⁺, 100%).

### (S)-N-(2-(4-(1-Acetyl-2-methyl-1,2,3,4-tetrahydroquinolin-6-yl)benzamido)ethyl)-6-bromo-3-methyl-8-morpholinoimidazo[1,2-a]pyrazine-2-carboxamide (S17.i[Cpd 021])

A solution of N-(2-aminoethyl)-6-bromo-3-methyl-8-morpholinoimidazo[1,2-a]pyrazine-2-carboxamide (S14.4) (110 mg, 0.29 mmol), (S)-4-(1-acetyl-2-methyl-1,2,3,4-tetrahy-droquinolin-6-yl)benzoic acid (S9.8) (115 mg, 0.37 mmol), HCTU (237 mg, 0.57 mmol), DIPEA (0.30 mL, 1.72 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as an off-white powder (184 mg, 95%). ¹H NMR (401 MHz, CDCl₃) δ 7.96 (br s, 1H), 7.87 (d, J=8.2 Hz, 2H), 7.75 (br s, 1H), 7.57 (d, J=8.2 Hz, 2H), 7.39 (d, J=8.2 Hz, 1H), 7.36 (s, 1H), 7.30 (s, 1H), 7.20 (br s, 1H), 4.78 (br s, 1H), 4.28 (br s, 4H), 3.79 (app. t, 4H), 3.69 (br s, 4H), 2.71-2.62 (m, 1H), 2.66 (s, 3H), 2.60-2.50 (m, 1H), 2.39-2.27 (m, 1H), 2.16 (s, 3H), 1.37 (br s, 1H), 1.12 (d, J=6.4 Hz, 3H). LCMS: (Method A) Rf=5.005 min, (ESI) m/z: 674.2, 676.2 ([M+H]⁺, 100%).

### (S)-N-(2-(4-(1-Acetyl-2-methyl-1,2,3,4-tetrahydroquinolin-6-yl)benzamido)ethyl)-6-(2-aminopyrimidin-5-yl)-3-methyl-8-morpholinoimidazo[1,2-a]pyrazine-2-carboxamide [Cpd 021]

A solution of (S)-N-(2-(4-(1-acetyl-2-methyl-1,2,3,4-tetrahydroquinolin-6-yl)benzamido)ethyl)-6-bromo-3-methyl-8-morpholinoimidazo[1,2-a]pyrazine-2-carboxamide (S17.i[Cpd 021]) (80 mg, 0.12 mmol), 2-aminopyrimidine-5-boronic acid (20 mg, 0.14 mmol), K₂CO₃ (49 mg, 0.35 mmol) in a mixture of DME/H₂O (4:1, 5 mL) was degassed with N₂ for 15 min. Pd(dppf)Cl₂ (12 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The organic extracts were combined, dried over MgSO₄ and concentrated in vacuo. The resulting residue was purified using flash column chromatography (0-8% MeOH in DCM) to give a brown solid, which was triturated with cold MeOH to give the title compound as a white powder (65 mg, 80%). ¹H NMR (401 MHz, DMSO) δ 8.90 (s, 2H), 8.72 (br t, J=4.8 Hz, 1H), 8.53 (br t, J=4.8 Hz, 1H), 8.19 (s, 1H), 7.94 (d, J=8.5 Hz, 2H), 7.79 (d, J=8.5 Hz, 2H), 7.61 (s, 1H), 7.57 (dd, J=8.5, 1.6 Hz, 1H), 7.46 (br s, 1H), 6.87 (s, 2H), 4.72-4.56 (m, 1H), 4.30 (br s, 4H), 3.77 (app. t, 4H), 3.50 (br s, 4H), 2.81-2.69 (m, 4H), 2.62-2.53 (m, 1H), 2.35-2.23 (m, 1H), 2.12 (s, 3H), 1.36 (br s, 1H), 1.07 (d, J=6.4 Hz, 3H). LCMS: (Method A) Rf=4.324 min, (ESI) m/z: 689.0 ([M+H]⁺, 100%). HRMS (ESI⁺) calcd for [C₃₇H₄₀N₁₀O₄+H]⁺689.3307, found 689.3329.

### Ethyl 2-(6-chloro-8-morpholinoimidazo[1,2-a]pyrazin-2-yl)acetate (514.5)

A suspension of 5-bromo-3-morpholinopyrazin-2-amine (S14.1) (500 mg, 1.93 mmol) and ethyl 4-chloroacetoacetate (1.25 g, 7.59 mmol) was heated in a sealed vial at 70° C. o.n. The solidified crude material was dissolved in DCM, washed with sat. bicarb solution (×2), dried over MgSO₄ and concentrated in vacuo. The residue was then triturated with diethyl ether and filtered to give the crude product as a brown powder (275 mg), which was used directly in the next step without further purification. ¹H NMR (401 MHz, CDCl₃) δ 7.48 (s, 1H), 7.47 (s, 1H), 4.31 (app. t, 4H), 4.20 (q, J=7.1 Hz, 2H), 3.84 (app. t, 4H), 3.82 (s, 2H), 1.29 (t, J=7.1 Hz, 3H). LCMS: (Method B) Rf=3.309 min, (ESI) m/z: 325.1 ([M+H]⁺, 100%).

### Lithium(I) 2-(6-chloro-8-morpholinoimidazo[1,2-a]pyrazin-2-yl)acetate (S14.6)

The crude ethyl 2-(6-chloro-8-morpholinoimidazo[1,2-a]pyrazin-2-yl)acetate (S14.5) (275 mg) from the last step was re-dissolved in a mixture a THE/water (4:1, 5 mL). To this solution was added LiOH monohydrate (53 mg, 1.26 mmol) and the reaction mixture was heated to reflux o.n. The THE was removed in vacuo and to the aqueous residue was added acetone. The resulting precipitate was filtered to give the title compound as a light yellow pow der (237 mg, 41% over two steps). ¹H NMR (401 MHz, D₂O) δ 7.51 (s, 1H), 7.42 (s, 1H), 3.92-3.86 (m, 4H), 3.86-3.80 (m, 4H), 3.60 (s, 2H). LCMS: (Method C) Rf=3.232 min, (ESI) m/z: 297.1 ([M+H]⁺, 100%).

### (S)-N-(4-(1-Acetyl-2-methyl-1,2,3,4-tetrahydroquinolin-6-yl)phenyl)-2-(6-chloro-8-morpholinoimidazo[1,2-a]pyrazin-2-yl)acetamide (S17.i[Cpd 022])

A solution of (S)-1-(6-(4-aminophenyl)-2-methyl-3,4-dihydroquinolin-1(2H)-yl)ethan-1-one (S9.14) (150 mg, 0.54 mmol), lithium(I) 2-(6-chloro-8-morpholinoimidazo[1,2-a]pyra-zin-2-yl)acetate (S14.6) (243 mg, 0.80 mmol), HCTU (443 mg, 1.07 mmol), DIPEA (0.56 mL, 3.25 mmol) in DMF (5 mL) was stirred at r.t o.n. The reaction mixture was then poured into 5% Na₂CO₃ solution, the resulting precipitate was filtered and purified using flash column chromatography (0-2% MeOH in DCM) to give an oil, which was triturated with EA and PE to give the title compound as a white powder (226 mg, 76%). ¹H NMR (401 MHz, CDCl₃) δ 9.23 (s, 1H), 7.55 (s, 4H), 7.52 (s, 1H), 7.45 (s, 1H), 7.40 (dd, J=8.2, 1.9 Hz, 1H), 7.36 (s, 1H), 7.18 (br s, 1H), 4.84 (br s, 1H), 4.37 (app. t, 4H), 3.90 (app. t, 4H), 3.87 (s, 2H), 2.68 (dt, J=14.5, 4.8 Hz, 1H), 2.62-2.51 (m, 1H), 2.36 (ddt, J=10.1, 7.2, 5.0 Hz, 1H), 2.18 (s, 3H), 1.40 (br s, 1H), 1.15 (d, J=6.5 Hz, 3H). LCMS: (Method B) Rf=3.000 min, (ESI) m/z: 559.2 ([M+H]⁺, 100%).

### (S)-N-(4-(1-Acetyl-2-methyl-1,2,3,4-tetrahydroquinolin-6-yl)phenyl)-2-(6-(2-aminopy-rimidin-5-yl)-8-morpholinoimidazo[1,2-a]pyrazin-2-yl)acetamide [Cpd 022]

A solution of (S)-N-(4-(1-acetyl-2-methyl-1,2,3,4-tetrahydroquinolin-6-yl)phenyl)-2-(6-chloro-8-morpholinoimidazo[1,2-a]pyrazin-2-yl)acetamide (S17.i[Cpd 022]) (100 mg, 0.18 mmol), 2-aminopyrimidine-5-boronic acid (38 mg, 0.27 mmol), K₂CO₃ (75 mg, 0.54 mmol) in a mixture of DME/H₂O (4:1, 5 mL) was degassed with N₂ for 15 min. Pd(dppf)Cl₂ (27 mg, 0.04 mmol) was then added and the mixture was further degassed for another 15 min. The reaction mixture was heated at 135° C. under microwave irradiation (fixed powder: 100 W) for 2 hr. The mixture was then diluted with water and extracted with DCM (×2). The organic extracts were combined, dried over MgSO₄ and concentrated in vacuo. The resulting residue was purified using flash column chromatography (0-5% MeOH in DCM) to give a brown solid, which was purified again using flash column chromatography (isocratic 3% MeOH (1% 7N NH₃ in MeOH) in DCM) to give the title compound as a light yellow powder (65 mg, 59%). ¹H NMR (401 MHz, CDCl₃) δ 9.40 (br s, 1H), 8.80 (s, 2H), 7.80 (s, 1H), 7.62-7.52 (m, 4H), 7.51 (s, 1H), 7.40 (dd, J=8.2, 2.0 Hz, 1H), 7.37 (br s, 1H), 7.18 (s, 1H), 5.18 (s, 2H), 4.84 (br s, 1H), 4.41 (app. t, 4H), 3.95 (app. t, 4H), 3.89 (s, 2H), 2.73-2.63 (m, 1H), 2.63-2.51 (m, 1H), 2.42-2.32 (m, 1H), 2.18 (s, 3H), 1.39 (br s, 1H), 1.15 (d, J=6.5 Hz, 3H). LCMS: (Method B) Rf=2.616 min, (ESI) m/z: 618.3 ([M+H]⁺, 100%). HRMS (ESI⁺) calcd for [C₃₄H₃₅N₉O₃+H]⁺618.2936, found 618.2952.

### (S)-N-(3-(1-Acetyl-2-methyl-1,2,3,4-tetrahydroquinolin-6-yl)phenyl)-2-(6-chloro-8-morpholinoimidazo[1,2-a]pyrazin-2-yl)acetamide (S17.i[Cpd 023])

A solution of (S)-1-(6-(3-aminophenyl)-2-methyl-3,4-dihydroquinolin-1(2H)-yl)ethan-1-one (S9.15) (150 mg, 0.54 mmol), lithium(I) 2-(6-chloro-8-morpholinoimidazo[1,2-a]pyra-zin-2-yl)acetate (S14.6) (243 mg, 0.80 mmol), HCTU (443 mg, 1.07 mmol), DIPEA (0.56 mL, 3.25 mmol) in DMF (5 mL) was stirred at r.t o.n. The reaction mixture was then poured into 5% Na₂CO₃ solution, the resulting precipitate was filtered and purified using flash column chromatography (0-2% MeOH in DCM) to give an oil, which was triturated with EA and PE to give the title compound as a white powder (225 mg, 75%). ¹H NMR (401 MHz, CDCl₃) δ 9.24 (s, 1H), 7.81 (s, 1H), 7.52 (s, 1H), 7.45 (s, 1H), 7.43-7.35 (m, 4H), 7.34-7.29 (m, 1H), 7.19 (br s, 1H), 4.83 (br s, 1H), 4.35 (app. t, 4H), 3.92-3.82 (m, 6H), 2.68 (dt, J=14.7, 4.9 Hz, 1H), 2.62-2.51 (m, 1H), 2.36 (ddt, J=10.2, 7.3, 5.1 Hz, 1H), 2.18 (s, 3H), 1.40 (br, 1H), 1.15 (d, J=6.5 Hz, 3H). LCMS: (Method B) Rf=3.034 min, (ESI) m/z: 559.2 ([M+H]⁺, 100%).

### (S)-N-(3-(1-Acetyl-2-methyl-1,2,3,4-tetrahydroquinolin-6-yl)phenyl)-2-(6-(2-aminopy-rimidin-5-yl)-8-morpholinoimidazo[1,2-a]pyrazin-2-yl)acetamide [Cpd 023]

A solution of (S)-N-(3-(1-acetyl-2-methyl-1,2,3,4-tetrahydroquinolin-6-yl)phenyl)-2-(6-chloro-8-morpholinoimidazo[1,2-a]pyrazin-2-yl)acetamide (S17.i[Cpd 023]) (100 mg, 0.18 mmol), 2-aminopyrimidine-5-boronic acid (38 mg, 0.27 mmol), K₂CO₃ (75 mg, 0.54 mmol) in a mixture of DME/H₂O (4:1, 5 mL) was degassed with N₂ for 15 min. Pd(dppf)Cl₂ (27 mg, 0.04 mmol) was then added and the mixture was further degassed for another 15 min. The reaction mixture was heated at 135° C. under microwave irradiation (fixed powder: 100 W) for 2 hr. The mixture was then diluted with water and extracted with DCM (×2). The organic extracts were combined, dried over MgSO₄ and concentrated in vacuo. The resulting residue was purified using flash column chromatography (0-5% MeOH in DCM) to give a brown solid, which was purified again using flash column chromatography (isocratic 3% MeOH (1% 7N NH₃ in MeOH) in DCM) to give the title compound as a pale yellow powder (48 mg, 43%). ¹H NMR (401 MHz, CDCl₃) δ 9.39 (s, 1H), 8.80 (s, 2H), 7.86-7.76 (m, 2H), 7.51 (s, 1H), 7.46-7.28 (m, 6H), 7.19 (br s, 1H), 5.33 (s, 2H), 4.84 (br s, 1H), 4.39 (app. t, 4H), 3.92 (app. t, 4H), 3.89 (s, 2H), 2.68 (dt, J=14.3, 4.6 Hz, 1H), 2.63-2.51 (m, 1H), 2.43-2.30 (m, 1H), 2.19 (s, 3H), 1.40 (br s, 1H), 1.15 (d, J=6.5 Hz, 3H). LCMS: (Method C) Rf=3.447 min, (ESI) m/z: 618.3 ([M+H]⁺, 100%). HRMS (ESI⁺) calcd for [C₃₄H₃₅N₉O₃+H]⁺618.2936, found 618.2959.

### (S)-N-(4-(1-Acetyl-2-methyl-1,2,3,4-tetrahydroquinolin-6-yl)benzyl)-2-(6-chloro-8-morpholinoimidazo[1,2-a]pyrazin-2-yl)acetamide (S17.i[Cpd 024])

To a solution of (S)-1-(6-(4-(aminomethyl)phenyl)-2-methyl-3,4-dihydroquinolin-1(2H)-yl)ethan-1-one (S9.12) (70 mg, 0.24 mmol) in DMF (5 mL) was added lithium(I) 2-(6-chloro-8-morpholinoimidazo[1,2-a]pyrazin-2-yl)acetate (S14.6) (80 mg, 0.26 mmol), EDC.HCl (81 mg, 0.20 mmol), HOBT (53 mg, 0.39 mmol) and DIPEA (0.14 mL, 0.77 mmol). The reaction mixture was stirred at r.t for 1.5 d and then poured into a 5% Na₂CO₃ solution. The resulting precipitate was filtered and purified using flash column chromatography (0-3% MeOH in DCM) to give an oil, which was triturated with EA and PE to give the title compound as an off-white powder (72 mg, 53%). ¹H NMR (401 MHz, CDCl₃) δ 7.53 (d, J=8.2 Hz, 2H), 7.49 (s, 1H), 7.42 (s, 1H), 7.39 (dd, J=8.1, 1.9 Hz, 1H), 7.35 (sd, J=1.6 Hz, 1H), 7.32 (d, J=8.3 Hz, 3H, overlapped with amide NH proton), 7.20 (br s, 1H), 4.84 (br s, 1H), 4.49 (d, J=5.5 Hz, 2H), 4.17 (app. t, 4H), 3.79 (s, 2H), 3.73 (app. t, 4H), 2.69 (dt, J=10.1, 5.3 Hz, 1H), 2.64-2.53 (m, 1H), 2.42-2.32 (m, 1H), 2.20 (s, 3H), 1.41 (br s, 1H), 1.17 (d, J=6.5 Hz, 3H). LCMS: (Method B) Rf=3.446 min, (ESI) m/z: 573.2 ([M+H]⁺, 100%).

### (S)-N-(4-(1-Acetyl-2-methyl-1,2,3,4-tetrahydroquinolin-6-yl)benzyl)-2-(6-(2-aminopy-rimidin-5-yl)-8-morpholinoimidazo[1,2-a]pyrazin-2-yl)acetamide [Cpd 024]

A solution (S)-N-(4-(1-acetyl-2-methyl-1,2,3,4-tetrahydroquinolin-6-yl)benzyl)-2-(6-chloro-8-morpholinoimidazo[1,2-a]pyrazin-2-yl)acetamide (S17.i[Cpd 024]) (25 mg, 0.04 mmol), 2-aminopyrimidine-5-boronic acid (13 mg, 0.09 mmol), K₂CO₃ (18 mg, 0.13 mmol) in a mixture of DME/H₂O (4:1, 5 mL) was degassed with N₂ for 15 min. Pd(dppf)Cl₂ (7 mg, 0.01 mmol) was then added and the mixture was further degassed for another 15 min. The reaction mixture was heated at 135° C. under microwave irradiation (fixed powder: 100 W) for 2 hr. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was purified again using flash column chromatography (0-4% MeOH (1% 7N NH₃ in MeOH) in DCM) to give the title compound as an off-white powder (12 mg, 44%). ¹H NMR (401 MHz, CDCl₃) δ 8.77 (s, 2H), 7.76 (s, 1H), 7.53 (d, J=8.0 Hz, 2H), 7.46 (s, 1H), 7.44-7.30 (m, 5H), 7.20 (br s, 1H), 5.20 (s, 2H), 4.84 (br s, 1H), 4.50 (d, J=5.4 Hz, 2H), 4.21 (app. t, 4H), 3.78 (s, 2H), 3.74 (app. t, 4H), 2.75-2.64 (m, 1H), 2.63-2.51 (m, 1H), 2.43-2.30 (m, 1H), 2.19 (s, 3H), 1.40 (br s, 1H), 1.16 (d, J=6.3 Hz, 3H). LCMS: (Method C) Rf=3.341 min, (ESI) m/z: 632.3 ([M+H]⁺, 100%). HRMS (ESI⁺) calcd for [C₃₅H₃₇N₉O₃+H]⁺632.3092, found 632.3101.

### (S)-N-(3-(1-Acetyl-2-methyl-1,2,3,4-tetrahydroquinolin-6-yl)benzyl)-2-(6-chloro-8-morpholinoimidazo[1,2-a]pyrazin-2-yl)acetamide (S17.i[Cpd 025])

To a solution of (S)-1-(6-(3-(aminomethyl)phenyl)-2-methyl-3,4-dihydroquinolin-1(2H)-yl)ethan-1-one (S9.13) (70 mg, 0.24 mmol) in DMF (5 mL) was added lithium(I) 2-(6-chloro-8-morpholinoimidazo[1,2-a]pyrazin-2-yl)acetate (S14.6) (80 mg, 0.26 mmol), EDC.HCl (81 mg, 0.20 mmol), HOBT (53 mg, 0.39 mmol) and DIPEA (0.14 mL, 0.77 mmol). The reaction mixture was stirred at r.t for 1.5 d and then poured into a 5% Na₂CO₃ solution. The resulting precipitate was filtered and purified using flash column chromatography (0-3% MeOH in DCM) to give an oil, which was triturated with DCM and PE to give the title compound as an off-white powder (80 mg, 59%). ¹H NMR (401 MHz, DMSO) δ 8.54 (t, J=5.9 Hz, 1H), 8.09 (s, 1H), 7.82 (s, 1H), 7.56-7.49 (m, 2H), 7.44 (s, 1H), 7.42-7.33 (m, 3H), 7.23 (d, J=7.6 Hz, 1H), 4.72-4.57 (m, 1H), 4.36 (d, J=5.8 Hz, 2H), 4.15 (app. t, 4H), 3.66 (app. t, 6H), 2.67 (dt, J=15.0, 5.3 Hz, 1H), 2.59-2.46 (m, 1H), 2.33-2.22 (m, 1H), 2.11 (s, 3H), 1.34 (br s, 1H), 1.06 (d, J=6.5 Hz, 3H). LCMS: (Method C) Rf=3.648 min, (ESI) m/z: 573.2 ([M+H]⁺, 100%).

### (S)-N-(3-(1-Acetyl-2-methyl-1,2,3,4-tetrahydroquinolin-6-yl)benzyl)-2-(6-(2-aminopy-rimidin-5-yl)-8-morpholinoimidazo[1,2-a]pyrazin-2-yl)acetamide [Cpd 025]

A solution (S)-N-(3-(1-Acetyl-2-methyl-1,2,3,4-tetrahydroquinolin-6-yl)benzyl)-2-(6-chloro-8-morpholinoimidazo[1,2-a]pyrazin-2-yl)acetamide (S17.i[Cpd 025]) (50 mg, 0.09 mmol), 2-aminopyrimidine-5-boronic acid (19 mg, 0.14 mmol), K₂CO₃ (36 mg, 0.26 mmol) in a mixture of DME/H₂O (4:1, 5 mL) was degassed with N₂ for 15 min. Pd(dppf)Cl₂ (13 mg, 0.02 mmol) was then added and the mixture was further degassed for another 15 min. The reaction mixture was heated at 135° C. under microwave irradiation (fixed powder: 100 W) for 2 hr. The mixture was then diluted with water and extracted with a mixture of DCM and isopropanol (3:1) (×2). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as an-off white powder (19 mg, 35%). ¹H NMR (401 MHz, CDCl₃) δ 8.76 (s, 2H), 7.75 (s, 1H), 7.55-7.32 (m, 8H), 7.19 (br s, 1H), 5.19 (s, 2H), 4.82 (br s, 1H), 4.52 (d, J=5.3 Hz, 2H), 4.14 (app. t, 4H), 3.78 (s, 2H), 3.66 (app. t, 4H), 2.66 (dt, J=14.5, 4.8 Hz, 1H), 2.61-2.50 (m, 1H), 2.42-2.29 (m, 1H), 2.18 (s, 3H), 1.40 (br s, 1H), 1.15 (d, J=6.5 Hz, 3H). LCMS: (Method C) Rf=3.286 min, (ESI) m/z: 632.3 ([M+H]⁺, 100%). HRMS (ESI⁺) calcd for [C₃₅H₃₇N₉O₃+H]⁺632.3092, found 632.3108.

### Thieno[3,2-d]pyrimidine-2,4(1H,3H)-dione (S15.1)

Synthesis as per WO2018/085342A1.

### 2,4-Dichlorothieno[3,2-d]pyrimidine (S15.2)

A solution of thieno[3,2-d]pyrimidine-2,4(1H,3H)-dione (S15.1) (5.9 g, 35.1 mmol) in POCl₃ (47 mL) was heated at 100° C. for 24 hr. The reaction mixture was slowly transferred into an ice-water mixture with vigorous stirring. The resulting suspension was stirred at 0° C. for 20 min and then filtered, washed with water, air-dried to give the title compound as an off-white powder (5.16 g. 72%). This product was used in the next step without further purification.

¹H NMR (401 MHz, CDCl₃): δ 8.06 (d, J=5.5 Hz, 1H), 7.50 (d, J=5.5 Hz, 1H). LCMS: (Method A) Rf=6.262 min, (ESI) m/z: 205.1 ([M+H]⁺, 100%).

### 4-(2-Chlorothieno[3,2-d]pyrimidin-4-yl)morpholine (S15.3)

Synthesis as per WO2011/130628A1.

### 2-Chloro-4-morpholinothieno[3,2-d]pyrimidine-6-carbaldehyde (S15.4)

Synthesis as per WO2018/085342A1.

### (2-Chloro-4-morpholinothieno[3,2-d]pyrimidin-6-yl)methanamine (S15.6)

Step 1: A solution of hydroxylamine hydrochloride (160 mg, 2.30 mmol) and sodium acetate (235 mg, 2.86 mmol) in 80% EtOH was stirred at r.t for 30 min. 2-chloro-4-morpholinothieno[3,2-d]pyrimidine-6-carbaldehyde (S15.4) (400 mg, 1.41 mmol) was then added and the reaction mixture was heated to reflux for another 30 min. The EtOH was removed in vacuo and to the aqueous residue was added water, the precipitate was filtered and air-dried to give the oxime intermediate as a cis and trans mixture (S15.5). Step 2: The oxime was re-suspended in MeOH (10 mL). To this suspension was added zinc dust (922 mg, 14.1 mmol) and ammonium formate (890 mg, 14.1 mmol). The reaction mixture was then heated to reflux for 3 hr. The mixture was then filtered and the filtrate concentrated in vacuo. The residue was taken up into 1 M HCl solution and extracted with DCM (×2). The aqueous layer was basified to pH=14 with 10% NaOH solution and extracted with DCM (×2). The organic extracts were combined, dried over MgSO₄ and concentrated in in vacuo to give the title compound as an off-white powder (296 mg, 74%). ¹H NMR (401 MHz, DMSO) δ 7.20 (st, J=1.2 Hz, 1H), 4.04 (sd, J=1.2 Hz, 2H), 3.89 (app. t, 4H), 3.74 (app .t, 4H), 2.26 (br s, 2H). LCMS: (Method A) Rf=0.536 min, (ESI) m/z: 285.0 ([M+H]⁺, 100%).

### (S)-4-(1-Acetyl-2-methyl-1,2,3,4-tetrahydroquinolin-6-yl)-N-((2-chloro-4-morpholinothieno[3,2-d]pyrimidin-6-yl)methyl)benzamide (S17.i[Cpd 026])

A solution of (2-chloro-4-morpholinothieno[3,2-d]pyrimidin-6-yl)methanamine (515.6) (90 mg, 0.32 mmol), (S)-4-(1-acetyl-2-methyl-1,2,3,4-tetrahydroquinolin-6-yl)benzoic acid (S9.8) (117 mg, 0.38 mmol), HCTU (262 mg, 0.63 mmol), DIPEA (0.33 mL, 1.89 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as a light yellow powder (101 mg, 55%). ¹H NMR (401 MHz, CDCl₃) δ 7.93 (d, J=8.4 Hz, 2H), 7.66 (br, J=8.3 Hz, 2H), 7.45 (dd, J=8.2, 2.0 Hz, 1H), 7.41 (br s, 1H), 7.26 (br s overlapped with CHCl₃ peak, 1H)7.24 (s, 1H), 7.20-7.12 (m, 1H), 4.90 (d, J=6.0 Hz, 2H), 4.81 (br s, 1H), 3.99-3.90 (app. t, 4H), 3.84-3.75 (app. t, 4H), 2.71 (dt, J=14.9, 5.1 Hz, 1H), 2.60 (ddd, J=14.8, 10.4, 4.7 Hz, 1H), 2.43-2.31 (m, 1H), 2.19 (s, 3H), 1.42 (br s, 1H). LCMS: (Method A) Rf=6.092 min, (ESI) m/z: 576.2 ([M+H]⁺, 100%).

### (S)-4-(1-Acetyl-2-methyl-1,2,3,4-tetrahydroquinolin-6-yl)-N-((2-(2-aminopyrimidin-5-yl)-4-morpholinothieno[3,2-d]pyrimidin-6-yl)methyl)benzamide [Cpd 026]

A solution of (S)-4-(1-acetyl-2-methyl-1,2,3,4-tetrahydroquinolin-6-yl)-N-((2-chloro-4-morpholinothieno[3,2-d]pyrimidin-6-yl)methyl)benzamide (S17.i[Cpd 026]) (70 mg, 0.12 mmol), 2-aminopyrimidine-5-boronic acid (25 mg, 0.18 mmol), K₂CO₃ (50 mg, 0.36 mmol) in a mixture of DME/H₂O (4:1, 5 mL) was degassed with N₂ for 15 min. Pd(dppf)Cl₂ (18 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×3). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as a white powder (60 mg, 78%). ¹H NMR (401 MHz, CDCl₃) δ 9.24 (s, 2H), 7.92 (d, J=8.4 Hz, 2H), 7.67 (d, J=8.4 Hz, 2H), 7.45 (dd, J=8.2, 1.8 Hz, 1H), 7.42 (s, 1H), 7.35 (s, 1H), 7.23 (br s, 1H), 6.98 (br t, J=5.7 Hz, 1H), 5.35 (s, 2H), 4.95 (d, J=5.8 Hz, 2H), 4.82 (br s, 1H), 3.98 (app. t, 4H), 3.85 (app. t, 4H), 2.70 (dt, J=14.2, 4.7 Hz, 1H), 2.65-2.53 (m, 1H), 2.44-2.31 (m, 1H), 2.20 (s, 3H), 1.42 (br s, 2H), 1.16 (d, J=6.5 Hz, 3H). LCMS: (Method A) Rf=5.187 min, (ESI) m/z: 635.3 ([M+H]⁺, 100%). HRMS (ESI⁺) calcd for [C₃₄H₃₄N₈O₃S+H]⁺635.2547, found 635.2575.

### (S)-2-(4-(1-Acetyl-2-methyl-1,2,3,4-tetrahydroquinolin-6-yl)phenyl)-N-((2-chloro-4-morpholinothieno[3,2-d]pyrimidin-6-yl)methyl)acetamide (S17.i[Cpd 027])

To a solution of (2-chloro-4-morpholinothieno[3,2-d]pyrimidin-6-yl)methanamine (S15.6) (100 mg, 0.35 mmol) in DMF (5 mL) was (S)-2-(4-(1-acetyl-2-methyl-1,2,3,4-tetrahy-droquinolin-6-yl)phenyl)acetic acid (S9.10) (136 mg, 0.42 mmol), EDC.HCl (101 mg, 0.53 mmol), HOBT (71 mg, 0.53 mmol) and DIPEA (0.18 mL, 1.15 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as a white powder (180 mg, 87%). ¹H NMR (401 MHz, CDCl₃) δ 7.56 (d, J=8.2 Hz, 2H), 7.40 (dd, J=8.2, 1.9 Hz, 1H), 7.37 (s, 1H), 7.34 (d, J=8.2 Hz, 2H), 7.21 (br s, 1H), 7.09 (s, 1H), 6.26 (br t, J=5.9 Hz, 1H), 4.82 (br s, 1H), 4.65 (d, J=6.0 Hz, 2H), 3.94 (app. t, 4H), 3.79 (app. t, 4H), 3.69 (s, 2H), 2.69 (dt, J=14.5, 4.9 Hz, 1H), 2.64-2.52 (m, 1H), 2.42-2.31 (m, 1H), 2.18 (s, 3H), 1.40 (br s, 1H), 1.15 (d, J=6.5 Hz, 3H). LCMS: (Method A) Rf=4.753 min, (ESI) m/z: 589.9, 591.9 ([M+H]⁺, 100%).

### (S)-2-(4-(1-Acetyl-2-methyl-1,2,3,4-tetrahydroquinolin-6-yl)phenyl)-N-((2-(2-aminopy-rimidin-5-yl)-4-morpholinothieno[3,2-d]pyrimidin-6-yl)methyl)acetamide [Cpd 027]

A solution of (S)-2-(4-(1-acetyl-2-methyl-1,2,3,4-tetrahydroquinolin-6-yl)phenyl)-N-((2-chloro-4-morpholinothieno[3,2-d]pyrimidin-6-yl)methyl)acetamide (S17.i[Cpd 027]) (80 mg, 0.14 mmol), 2-aminopyrimidine-5-boronic acid (37 mg, 0.27 mmol), K₂CO₃ (56 mg, 0.41 mmol) in a mixture of DME/H₂O (4:1, 5 mL) was degassed with N₂ for 15 min. Pd(dppf)Cl₂ (20 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with a mixture of DCM/isoproanol (3:1, ×3). The organic extracts were combined, dried over MgSO₄ and concentrated in vacuo. The resulting residue was purified using flash column chromatography (0-6% MeOH in DCM) to give a yellow solid, which was triturated with cold MeOH to give the title compound as an off-white powder (67 mg, 76%). ¹H NMR (401 MHz, DMSO) δ 9.09 (s, 2H), 8.92 (br t, J=5.9 Hz, 1H), 7.62 (d, J=8.3 Hz, 2H), 7.54-7.47 (m, 2H), 7.42 (br s, 1H), 7.38 (d, J=8.3 Hz, 2H), 7.28 (s, 1H), 7.09 (s, 2H), 4.65 (q, J=6.4 Hz, 1H), 4.60 (d, J=5.8 Hz, 2H), 3.87 (app. t, 4H), 3.70 (app. t, 4H), 3.55 (s, 2H), 2.71 (dt, J=15.0, 5.3 Hz, 1H), 2.60-2.52 (m, 1H), 2.35-2.23 (m, 1H), 2.11 (s, 3H), 1.34 (br s, 1H), 1.06 (d, J=6.5 Hz, 3H). LCMS: (Method A) Rf=4.123 min, (ESI) m/z: 649.0 ([M+H]⁺, 100%). HRMS (ESI⁺) calcd for [C₃₅H₃₆N₈O₃S+H]⁺649.2704, found 649.2728.

### tert-Butyl 4-((2-chloro-4-morpholinothieno[3,2-d]pyrimidin-6-yl)methyl)piperazine-1-carboxylate (S15.7)

To a solution of 2-chloro-4-morpholinothieno[3,2-d]pyrimidine-6-carbaldehyde (S15.4) (400 mg, 1.41 mmol) in dry DCE (15 mL) was added Boc-piperazine (520 mg, 2.79 mmol), sodium triacetoxyborohydride (613 mg, 2.89 mmol) and 3 Å MS. The reaction vessel was then evacuated and backfilled with N₂ three times. The reaction mixture was stirred at r.t under N₂ for 2 d. The reaction mixture was then filtered through celite and washed with DCM. The solvent was then concentrated in vacuo and the residue purified using flash column chromatography (20% EA in DCM then 2% MeOH in DCM) to give the title compound as a bright yellow powder (505 mg, 79%). ¹H NMR (401 MHz, CDCl₃) δ 7.12 (s, 1H), 3.95 (app. t, 4H), 3.80 (app. t, 4H), 3.77 (s, 2H), 3.43 (app. t, 4H), 2.46 (app. t, 4H), 1.42 (s, 9H). LCMS: (Method A) Rf=5.152 min, (ESI) m/z: 454.2 ([M+H]⁺, 100%).

### tert-Butyl 4-((2-(2-aminopyrimidin-5-yl)-4-morpholinothieno[3,2-d]pyrimidin-6-yl)methyl)piperazine-1-carboxylate (S15.8)

A solution of tert-butyl 4-((2-chloro-4-morpholinothieno[3,2-d]pyrimidin-6-yl)methyl)piperazine-1-carboxylate (S15.7) (400 mg, 0.88 mmol), 2-aminopyrimidine-5-boronic acid (184 mg, 1.32 mmol), K₂CO₃ (365 mg, 2.64 mmol) in a mixture of DME/H₂O (4:1, 10 mL) was degassed with N₂ for 15 min. Pd(dppf)Cl₂ (129 mg, 0.18 mmol) was then added and the mixture was further degassed for another 15 min. The reaction vessel was then evacuated and backfilled with N₂ three times. The reaction mixture was heated to reflux under N₂ o.n. The mixture was then diluted with water and acidified to pH=1 and extracted with DCM (×2). The aqueous layer was then filtered through celite and the filtrated basified to pH=14 with 10% NaOH solution. The resulting precipitate was filtered and re-dissolved in a mixture of DCM/isopropanol (3:1). The organic was filtered again and the filtrate concentrated in vacuo. The residue was triturated with cold isopropanol to give the title compound as an off-white powder (350 mg, 78%). ¹H NMR (401 MHz, CDCl₃) δ 9.24 (s, 2H), 7.24 (s, 1H), 5.66 (s, 2H), 3.99 (br s, 4H), 3.85 (br s, 4H), 3.80 (s, 2H), 3.45 (br s, 4H), 2.48 (br s, 4H), 1.43 (s, 9H). LCMS: (Method A) Rf=1.908 min, (ESI) m/z: 513.3 ([M+H]⁺, 100%).

### 5-(4-Morpholino-6-(piperazin-1-ylmethyl)thieno[3,2-d]pyrimidin-2-yl)pyrimidin-2-amine (di-TFA salt) (S15.9)

A solution of tert-butyl 4-((2-(2-aminopyrimidin-5-yl)-4-morpholinothieno[3,2-d]py-rimidin-6-yl)methyl)piperazine-1-carboxylate (S15.8) (260 mg, 0.51 mmol) in a mixture of DCM/TFA (1:1, 6 mL) was stirred at r.t for 1 hr. The solvent was then concentrated in vacuo. The resulting residue was then triturated with MeOH and diethyl ether to give the title compound as an off-white powder (325 mg, 98%). ¹H NMR (401 MHz, MeOD) δ 9.14 (s, 2H), 7.40 (s, 1H), 4.17 (app. t, 4H), 4.04 (s, 2H), 3.89 (app. t, 4H), 3.29 (app. t, 4H), 2.85 (app. t, 4H). LCMS: (Method A) Rf=4.153 min, (ESI) m/z: 413.2 ([M+H]⁺, 100%).

### (S)-1-(6-(4-(4-((2-(2-Aminopyrimidin-5-yl)-4-morpholinothieno[3,2-d]pyrimidin-6-yl)methyl)piperazine-1-carbonyl)phenyl)-2-methyl-3,4-dihydroquinolin-1(2H)-yl)ethan-1-one [Cpd 028]

A solution of (S)-4-(1-acetyl-2-methyl-1,2,3,4-tetrahydroquinolin-6-yl)benzoic acid (S9.8) (72 mg, 0.23 mmol), HCTU (129 mg, 0.31 mmol), DIPEA (0.22 mL, 1.26 mmol) in DMF (5 mL) was stirred at r.t for 15 min. To this solution was then added 5-(4-morpholino-6-(piperazin-1-ylmethyl)thieno[3,2-d]pyrimidin-2-yl)pyrimidin-2-amine (di-TFA salt) (515.9) (100 mg, 0.16 mmol) and reaction mixture was allowed to stir at r.t o.n. The reaction mixture was then poured into 5% Na₂CO₃ solution, the resulting precipitate was filtered and purified using flash column chromatography (0-4% MeOH in DCM) to give an oil, which was triturated with DCM and PE to give the title compound as a white powder (72 mg, 65%). ¹H NMR (401 MHz, CDCl₃) δ 9.27 (s, 2H), 7.61 (d, J=8.3 Hz, 2H), 7.48 (d, J=8.3 Hz, 2H), 7.42 (dd, J=8.2, 2.0 Hz, 1H), 7.38 (br s, 1H), 7.29 (s, 1H), 7.23 (br s, 1H), 5.33 (s, 2H), 4.83 (br s, 1H), 4.07-3.97 (m, 4H), 3.96-3.75 (m, 8H), 3.57 (br s, 2H), 2.77-2.45 (m, 6H), 2.42-2.32 (m, 1H), 2.19 (s, 3H), 1.41 (br s, 1H), 1.16 (d, J=6.5 Hz, 3H). LCMS: (Method A) Rf=4.895 min, (ESI) m/z: 704.3 ([M+H]⁺, 100%). HRMS (ESI⁺) calcd for [C₃₈H₄₁N₉O₃S+H]⁺704.3126, found 704.3140.

### (S)-1-(6-(4-((4-((2-(2-Aminopyrimidin-5-yl)-4-morpholinothieno[3,2-d]pyrimidin-6-yl)methyl)piperazin-1-yl)methyl)phenyl)-2-methyl-3,4-dihydroquinolin-1(2H)-yl)ethan-1-one [Cpd 029]

A solution of 5-(4-morpholino-6-(piperazin-1-ylmethyl)thieno[3,2-d]pyrimidin-2-yl)pyrimidin-2-amine (di-TFA salt) (S15.9) (100 mg, 0.16 mmol), (S)-4-(1-acetyl-2-methyl-1,2,3,4-tetrahydroquinolin-6-yl)benzaldehyde (S9.7) (92 mg, 0.31 mmol) and glacial acetic acid (0.18 mL, 3.08 mmol) in dry DCE (15 mL) was stirred at r.t for 15 min. To this mixture was then added sodium triacetoxyborohydride (102 mg, 0.48 mmol). The reaction vessel was then evacuated and backfilled with N₂ three times. The reaction mixture was heated to reflux under N₂ o.n. Another portion of sodium triacetoxyborohydride (102 mg, 0.48 mmol) was added and the reaction mixture was allowed to stir for another 12 hr. The reaction mixture was diluted with DCM, washed with 10% NaOH solution, dried over MgSO₄ and the solvent concentrated in vacuo. The residue was then purified using flash column chromatography (0-7% MeOH (1% 7N NH₃ in MeOH) in DCM) to give an oil, which was triturated with DCM and PE to give the title compound as a light yellow powder (49 mg, 43%). ¹H NMR (401 MHz, CDCl₃) δ 9.27 (s, 2H), 7.53 (d, J=8.1 Hz, 2H), 7.44-7.35 (m, 4H), 7.26 (s, 1H, partially overlapped with the solvent residue peak), 7.19 (br s, 1H), 5.33 (s, 2H), 4.83 (br s, 1H), 4.10-3.98 (m, 4H), 3.91-3.85 (m, 4H), 3.83 (s, 2H), 3.59 (s, 2H), 2.78-2.43 (m, 10H), 2.42-2.31 (m, 1H), 2.19 (s, 3H), 1.40 (br s, 1H), 1.15 (d, J=6.5 Hz, 3H). LCMS: (Method A) Rf=4.555 min, (ESI) m/z: 690.4 ([M+H]⁺, 100%). HRMS (ESI⁺) calcd for [C₃₈H₄₃N₉O₂S+H]⁺690.3333, found 690.3335.

### 2-Chloro-4-morpholinothieno[3,2-d]pyrimidine-6-carboxylic acid (515.10)

To a solution of 2-chloro-4-morpholinothieno[3,2-d]pyrimidine-6-carbaldehyde (S15.4) (400 mg, 1.41 mmol) in DCM (10 mL) was added m-CPBA (75%, 487 mg, 2.11 mmol). The reaction mixture was concentrated and the uniform mixture of solid was heated at 60° C. for 24 hr. The solid was triturated with DCM repeatedly to remove the 3-chlorobenzoic acid and the product was obtained as a yellow powder (292 mg, 69%). ¹H NMR (401 MHz, DMSO) δ 7.89 (s, 1H), 3.96-3.90 (m, 4H), 3.81-3.70 (m, 4H). LCMS: (Method A) Rf=6.339 min, (ESI) m/z: 300.1 ([M+H]⁺, 100%).

### (S)-N-(4-(1-Acetyl-2-methyl-1,2,3,4-tetrahydroquinolin-6-yl)benzyl)-2-chloro-4-morpholinothieno[3,2-d]pyrimidine-6-carboxamide (S17.i[Cpd 030])

A solution of (S)-1-(6-(4-(aminomethyl)phenyl)-2-methyl-3,4-dihydroquinolin-1(2H)-yl)ethan-1-one (S9.12) (150 mg, 0.51 mmol), 2-chloro-4-morpholinothieno[3,2-d]pyrimidine-6-carboxylic acid (S15.10) (183 mg, 0.61 mmol), HCTU (421 mg, 1.02 mmol), DIPEA (0.53 mL, 3.06 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as an off-white powder (87 mg, 30%). ¹H NMR (401 MHz, CDCl₃) δ 7.63 (s, 1H), 7.57 (d, J=8.2 Hz, 2H), 7.43 (d, J=8.2 Hz, 2H), 7.40-7.35 (m, 2H), 7.19 (br s, 1H), 6.84 (br t, J=5.3 Hz, 1H), 4.82 (br s, 1H), 4.70 (d, J=5.7 Hz, 2H), 4.03 (app. t, 4H), 3.85 (app. t, 4H), 2.69 (dt, J=14.7, 4.9 Hz, 1H), 2.64-2.53 (m, 1H), 2.43-2.31 (m, 1H), 2.16 (s, 3H), 1.40 (br s, 1H), 1.16 (d, J=6.5 Hz, 3H). LCMS: (Method A) Rf=6.312 min, (ESI) m/z: 576.2 ([M+H]⁺, 100%).

### (S)-N-(4-(1-Acetyl-2-methyl-1,2,3,4-tetrahydroquinolin-6-yl)benzyl)-2-(2-aminopyrim-idin-5-yl)-4-morpholinothieno[3,2-d]pyrimidine-6-carboxamide [Cpd 030]

A solution of (S)-N-(4-(1-acetyl-2-methyl-1,2,3,4-tetrahydroquinolin-6-yl)benzyl)-2-chloro-4-morpholinothieno[3,2-d]pyrimidine-6-carboxamide (S17.i[Cpd 030]) (40 mg, 0.07 mmol), 2-aminopyrimidine-5-boronic acid (15 mg, 0.11 mmol), K₂CO₃ (29 mg, 0.21 mmol) in a mixture of DME/H₂O (4:1, 5 mL) was degassed with N₂ for 15 min. Pd(dppf)Cl₂ (10 mg, 0.01 mmol) was then added and the mixture was further degassed for another 15 min. The reaction vessel was then evacuated and backfilled with N₂ three times. The reaction mixture was heated to reflux under N₂ for 3 d. The mixture was then diluted with water and extracted with DCM (×3). The organic extracts were combined, dried over MgSO₄ and concentrated in vacuo. The resulting residue was purified using flash column chromatography (0-6% MeOH in DCM) to give a yellow solid, which was triturated with cold MeOH to give the title compound as a beige powder (12 mg, 27%). ¹H NMR (401 MHz, CDCl₃) δ 9.26 (s, 2H), 7.73 (s, 1H), 7.59 (d, J=8.2 Hz, 2H), 7.45 (d, J=8.2 Hz, 2H), 7.42 (dd, J=8.4, 2.1 Hz, 1H), 7.38 (br s, 1H), 7.20 (br s, 1H), 6.60 (br t, J=5.4 Hz, 1H), 5.26 (s, 2H), 4.83 (br s, 1H), 4.73 (d, J=5.7 Hz, 2H), 4.15-4.00 (m, 4H), 3.96-3.80 (m, 4H), 2.70 (dt, J=9.7, 4.6 Hz, 1H), 2.64-2.53 (m, 1H), 2.44-2.31 (m, 1H), 2.19 (s, 3H), 1.40 (br s, 1H), 1.16 (d, J=6.5 Hz, 3H). LCMS: (Method A) Rf=5.668 min, (ESI) m/z: 635.2 ([M+H]⁺, 100%). HRMS (ESI⁺) calcd for [C₃₄H₃₄N₈O₃S+H]⁺635.2547, found 635.2547.

### Methyl 2-chloro-4-morpholinothieno[3,2-d]pyrimidine-6-carboxylate (S15.11)

To a solution of 2-chloro-4-morpholinothieno[3,2-d]pyrimidine-6-carboxylic acid (S15.10) (350 mg, 1.17 mmol) in MeOH at 0° C. was added thionyl chloride (1.7 mL, 23.37 mmol) dropwise. The reaction mixture was then warmed up to r.t and stirred at this temperature for 3 d. The mixture was then concentrated to 5 mL and a 10% Na₂CO₃ solution was added. The resulting precipitate was filtered and purified using flash column chromatography (0-50% EA in PE) to give the title compound as a white powder (190 mg, 52%). ¹H NMR (401 MHz, CDCl₃) δ 7.91 (s, 1H), 4.04-3.99 (m, 4H), 3.96 (s, 3H), 3.87-3.82 (m, 4H). LCMS: (Method A) Rf=6.116 min, (ESI) m/z: 314.1 ([M+H]⁺, 100%).

### N-(2-Aminoethyl)-2-chloro-4-morpholinothieno[3,2-d]pyrimidine-6-carboxamide (S15.12)

To a solution of methyl 2-chloro-4-morpholinothieno[3,2-d]pyrimidine-6-carboxylate (S15.11) (150 mg, 0.48 mmol) in DCM (5 mL) was added ethylenediamine (3 mL). The reaction mixture was stirred at r.t for 24 hr. The residue was diluted with more DCM and washed with 10% NaOH solution (×2). The organic was dried over MgSO₄, concentrated in vacuo to give the title compound as an off-white powder (151 mg, 93%). ¹H NMR (401 MHz, CDCl₃) δ 7.63 (s, 1H), 7.19 (br t, J=4.4 Hz, 1H), 4.02 (app. t, 4H), 3.85 (app. t, 4H), 3.51 (dd, J=11.5, 5.6 Hz, 2H), 2.98 (app. t, 2H). LCMS: (Method A) Rf=4.354 min, (ESI) m/z: 342.1 ([M+H]⁺, 100%).

### (S)-N-(2-(4-(1-Acetyl-2-methyl-1,2,3,4-tetrahydroquinolin-6-yl)benzamido)ethyl)-2-chloro-4-morpholinothieno[3,2-d]pyrimidine-6-carboxamide (S17.i[Cpd 031])

A solution of N-(2-aminoethyl)-2-chloro-4-morpholinothieno[3,2-d]pyrimidine-6-carboxamide (S15.12) (120 mg, 0.35 mmol), (S)-4-(1-acetyl-2-methyl-1,2,3,4-tetrahydroquino-lin-6-yl)benzoic acid (S9.8) (163 mg, 0.53 mmol), HCTU (290 mg, 0.70 mmol), DIPEA (0.37 mL, 2.10 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as an off-white powder (185 mg, 83%). ¹H NMR (401 MHz, CDCl₃) δ 8.54 (br t, J=4.1 Hz, 1H), 7.88 (d, J=8.4 Hz, 2H), 7.80 (s, 1H), 7.62 (d, J=8.4 Hz, 2H), 7.42 (dd, J=8.2, 1.8 Hz, 1H), 7.39 (br s, 1H), 7.28 (br t, J=4.1 Hz, 1H), 7.24 (br s, 1H), 4.81 (br s, 1H), 4.00 (app. t, 4H), 3.82 (app. t, 4H), 3.80-3.69 (m, 4H), 2.70 (dt, J=14.8, 5.0 Hz, 1H), 2.65-2.52 (m, 1H), 2.42-2.30 (m, 1H), 2.19 (s, 3H), 1.42 (br s, 1H), 1.16 (d, J=6.5 Hz, 3H). LCMS: (Method A) Rf=5.868 min, (ESI) m/z: 633.2 ([M+H]⁺, 100%).

### (S)-N-(2-(4-(1-Acetyl-2-methyl-1,2,3,4-tetrahydroquinolin-6-yl)benzamido)ethyl)-2-(2-aminopyrimidin-5-yl)-4-morpholinothieno[3,2-d]pyrimidine-6-carboxamide[Cpd 031]

A solution of (S)-N-(2-(4-(1-acetyl-2-methyl-1,2,3,4-tetrahydroquinolin-6-yl)benzamido)ethyl)-2-chloro-4-morpholinothieno[3,2-d]pyrimidine-6-carboxamide (S17.i[Cpd 031]) (85 mg, 0.13 mmol), 2-aminopyrimidine-5-boronic acid (28 mg, 0.20 mmol), K₂CO₃ (56 mg, 0.41 mmol) in a mixture of DME/H₂O (4:1, 5 mL) was degassed with N₂ for 15 min. Pd(dppf)Cl₂ (20 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with a mixture of DCM and isopropanol (3:1) (×3). The organic extracts were combined, dried over MgSO₄ and concentrated in vacuo. The resulting residue was purified using flash column chromatography (0-8% MeOH in DCM) to give brown solid, which was triturated with cold MeOH to give the title compound as an off-white powder (44 mg, 47%).

¹H NMR (401 MHz, DMSO) δ 9.18-9.05 (m, 3H), 8.71 (br t, J=5.1 Hz, 1H), 8.06 (s, 1H), 7.94 (d, J=8.5 Hz, 2H), 7.79 (d, J=8.5 Hz, 2H), 7.60 (s, 1H), 7.57 (dd, J=8.4, 1.9 Hz, 1H), 7.45 (br d, J=6.8 Hz, 1H), 7.13 (s, 2H), 4.70-4.58 (m, 1H), 4.04-3.94 (m, 4H), 3.83-3.74 (m, 4H), 3.49 (br s, 4H), 2.74 (dt, J=14.9, 5.4 Hz, 1H), 2.62-2.53 (m, 1H), 2.35-2.23 (m, 1H), 2.12 (s, 3H), 1.36 (br s, 1H), 1.06 (d, J=6.5 Hz, 3H). LCMS: (Method B) Rf=2.554 min, (ESI) m/z: 346.7 ([M+2H]²+, 100%), 692.2 ([M+H]⁺, 40%). HRMS (ESI⁺) calcd for [C₃₆H₃₇N₉O₄S+H]⁺692.2762, found 692.2779.

### 7-Methylthieno[3,2-d]pyrimidine-2,4(1H,3H)-dione (516.1)

A mixture of methyl 3-aminothiophene-2-carboxylate (10 g, 58.41 mmol) and urea (17.5 g, 292 mmol) was heated at 190° C. for 3 hr. The reaction mixture was cooled to r.t and the resulting solid was dissolved in hot aq. NaOH solution (10%). The alkaline solution was filtered and the filtrate was neutralised to pH=7 with conc. HCl and stirred at 0° C. for 30 min. The resulting precipitate was filtered, washed with water and air-dried to give the title compound as an off-white powder (6.77 g, 64%). This product was used in the next step without further purification. ¹H NMR (401 MHz, DMSO) δ 11.41 (s, 1H), 11.21 (s, 1H), 7.70-7.67 (m, J=1.1 Hz, 1H), 2.19 (sd, J=1.1 Hz, 3H). LCMS: (Method A) Rf=5.795 min, (ESI) m/z: 140.1 ([(M-C═ONH)+H]⁺, 100%).

### 2,4-Dichloro-7-methylthieno[3,2-d]pyrimidine (S16.2)

Synthesis as per WO2014/0100366A1.

### 4-(2-Chloro-7-methylthieno[3,2-d]pyrimidin-4-yl)morpholine (S16.3)

Synthesis as per WO2014/0100366A1.

### Ethyl 2-chloro-7-methyl-4-morpholinothieno[3,2-d]pyrimidine-6-carboxylate (S16.4)

A solution of 4-(2-chloro-7-methylthieno[3,2-d]pyrimidin-4-yl)morpholine (S16.3) (2.0 g, 7.41 mmol) in dry THE (100 mL) was evacuated and backfilled with nitrogen 3 times. The solution was cooled to −78° C. and stirred for 10 min. To this solution was then dropwise added LDA (2 M in THF/Heptane/Ethylbenzene) (4.3 mL, 8.6 mmol) and the resulting mixture was stirred at −78° C. for 1.5 hr. To this reaction mixture was then quickly added ethyl chloroformate (4.2 mL, 44.4 mmol) with vigorous stirring. A large amount of precipitate was formed and the thick suspension was then stirred at this temperature for an additional 30 min. The suspension was then quenched by adding into an ice/0.5 M HCl solution mixture and stirred for 15 min. The resulting precipitate was filtered, air-dried and triturated with diethyl ether to get a bright-yellow powder (2.08 g, 82%). ¹H NMR (401 MHz, CDCl₃) δ 4.42 (q, J=7.1 Hz, 2H), 4.06-3.99 (m, 4H), 3.89-3.81 (m, 4H), 2.71 (s, 3H), 1.43 (t, J=7.1 Hz, 3H). LCMS: (Method A) Rf=5.524 min, (ESI) m/z: 341.9 ([M+H]⁺, 100%).

### Lithium(I) 2-chloro-7-methyl-4-morpholinothieno[3,2-d]pyrimidine-6-carboxylate (S16.5)

To a solution of ethyl 2-chloro-7-methyl-4-morpholinothieno[3,2-d]pyrimidine-6-carboxylate (S16.4) (500 mg, 0.1.46 mmol) in a mixture of THE and water (4:1, 10 mL) was added lithium hydroxide monohydrate (92 mg, 2.19 mmol). The reaction mixture was heated to reflux for 3 hr before cooling to r.t. The organic solvent was concentrated and to the residual aqueous layer was added acetone. The resulting precipitate was collected via filtration and dried under vacuum to give the title compound as an off-white powder (416 mg, 89%). ¹H NMR (400 MHz, D₂O) δ 3.84-3.76 (m, 4H), 3.73-3.64 (m, 4H), 2.18 (s, 3H). LCMS: (Method A) Rf=4.456 min, (ESI) m/z: 313.9 ([M+H]⁺, 100%).

### N-(2-Aminoethyl)-2-chloro-7-methyl-4-morpholinothieno[3,2-d]pyrimidine-6-carboxamide (516.6)

To a suspension of ethyl 2-chloro-7-methyl-4-morpholinothieno[3,2-d]pyrimidine-6-carboxylate (S16.4) in (200 mg, 0.59 mmol) in isopropanol (3 mL) was added ethylenediamine (3 mL). The reaction mixture was heated at 40-45° C. for 22 hr. The solvent was concentrated in vacuo and the residue was re-dissolved in DCM. The organic was washed with 5% NaOH solution (×2), dried over MgSO₄ and the solvent removed in vacuo. The residue was co-evaporated in DCM and diethyl ether (×2) to give the title compound as a yellow powder (196 mg, 94%). ¹H NMR (401 MHz, DMSO) δ 3.92 (app. t, 4H), 3.76 (app. t, 4H), 3.28 (app. t, 2H overlapped with H₂O peak), 2.70 (t, J=6.5 Hz, 2H), 2.46 (s, 3H). LCMS: (Method A) Rf=4.486 min, (ESI) m/z: 356.0 ([M+H]⁺, 100%).

### (S)-N-(4-(1-Acetyl-2-methyl-1,2,3,4-tetrahydroquinolin-6-yl)benzyl)-2-chloro-7-methyl-4-morpholinothieno[3,2-d]pyrimidine-6-carboxamide (S17.i[Cpd 032])

A solution of (S)-1-(6-(4-(aminomethyl)phenyl)-2-methyl-3,4-dihydroquinolin-1(2H)-yl)ethan-1-one (S9.12) (100 mg, 0.34 mmol), lithium(I) 2-chloro-7-methyl-4-morpholinothieno[3,2-d]pyrimidine-6-carboxylate (S16.5) (150 mg, 0.47 mmol), HCTU (211 mg, 0.51 mmol), DIPEA (0.36 mL, 2.06 mmol) in DMF (5 mL) was stirred at r.t o.n. Another portion of HCTU (211 mg, 0.51 mmol) was added and the reaction mixture was allowed to stir at r.t for another 4 hr. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as a white powder (170 mg, 85%). ¹H NMR (401 MHz, CDCl₃) δ 7.59 (d, J=8.2 Hz, 2H), 7.48-7.39 (m, 3H), 7.38 (br s, 1H), 7.21 (br s, 1H), 6.50 (t, J=5.4 Hz, 1H), 4.82 (br s, 1H), 4.71 (d, J=5.7 Hz, 2H), 4.00 (app. t, 4H), 3.83 (app. t, 4H), 2.69 (dt, J=10.1, 5.4 Hz, 1H), 2.63 (s, 3H), 2.64-2.51 (m, 1H), 2.43-2.31 (m, 1H), 2.18 (s, 3H), 1.41 (br s, 1H), 1.15 (d, J=6.5 Hz, 3H). LCMS: (Method A) Rf=5.258 min, (ESI) m/z: 590.2 ([M+H]⁺, 100%).

### (S)-N-(4-(1-Acetyl-2-methyl-1,2,3,4-tetrahydroquinolin-6-yl)benzyl)-2-(2-aminopyrim-idin-5-yl)-7-methyl-4-morpholinothieno[3,2-d]pyrimidine-6-carboxamide[Cpd 032]

A solution of (S)-N-(4-(1-acetyl-2-methyl-1,2,3,4-tetrahydroquinolin-6-yl)benzyl)-2-chloro-7-methyl-4-morpholinothieno[3,2-d]pyrimidine-6-carboxamide (S17.i[Cpd 032]) (80 mg, 0.14 mmol), 2-aminopyrimidine-5-boronic acid (28 mg, 0.20 mmol), K₂CO₃ (56 mg, 0.41 mmol) in a mixture of DME/H₂O (4:1, 5 mL) was degassed with N₂ for 15 min. Pd(dppf)Cl₂ (20 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×3). The organic extracts were combined, dried over MgSO₄ and concentrated in vacuo. The resulting residue was purified using flash column chromatography (0-6% MeOH in DCM) to give a dark yellow solid, which was triturated with cold MeOH to give the title compound as an off-white powder (54 mg, 61%). ¹H NMR (401 MHz, CDCl₃) δ 9.30 (s, 2H), 7.60 (d, J=8.2 Hz, 2H), 7.47 (d, J=8.2 Hz, 2H), 7.43 (dd, J=8.2, 1.9 Hz, 1H), 7.39 (s, 1H), 7.21 (br s, 1H), 6.48 (br t, J=5.4 Hz, 1H), 5.31 (s, 2H), 4.83 (br s, 1H), 4.74 (d, J=5.6 Hz, 2H), 4.04 (app. t, 4H), 3.87 (app. t, 4H), 2.79-2.64 (m, 4H), 2.63-2.52 (m, 1H), 2.44-2.31 (m, 1H), 2.19 (s, 3H), 1.41 (s, 1H), 1.16 (d, J=6.5 Hz, 3H). LCMS: (Method A) Rf=4.787 min, (ESI) m/z: 649.0 ([M+H]⁺, 100%). HRMS (ESI⁺) calcd for [C₃₅H₃₆N₈O₃S+H]⁺649.2704, found 649.2722.

### (S)-N-(2-(4-(1-Acetyl-2-methyl-1,2,3,4-tetrahydroquinolin-6-yl)benzamido)ethyl)-2-chloro-7-methyl-4-morpholinothieno[3,2-d]pyrimidine-6-carboxamide (S17.i[Cpd 033])

A solution of N-(2-aminoethyl)-2-chloro-7-methyl-4-morpholinothieno[3,2-d]pyrimi-dine-6-carboxamide (S16.6) (80 mg, 0.22 mmol), (S)-4-(1-acetyl-2-methyl-1,2,3,4-tetrahydro-quinolin-6-yl)benzoic acid (S9.8) (90 mg, 0.29 mmol), HCTU (186 mg, 0.45 mmol), DIPEA (0.24 mL, 1.38 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was co-evaporated with DCM and PE to give the title compound as a white powder (85 mg, 58%). ¹H NMR (401 MHz, DMSO) δ 8.65 (br t, J=4.7 Hz, 1H), 8.59 (br t, J=5.0 Hz, 1H), 7.93 (d, J=8.5 Hz, 2H), 7.79 (d, J=8.5 Hz, 2H), 7.60 (s, 1H), 7.57 (dd, J=8.3, 1.9 Hz, 1H), 7.46 (br d, J=7.1 Hz, 1H), 4.73-4.57 (m, 1H), 3.91 (app. t, 4H), 3.75 (app. t, 4H), 3.49 (br s, 4H), 2.81-2.69 (m, 1H), 2.62-2.53 (m, 1H), 2.47 (s, 3H), 2.36-2.22 (m, 1H), 2.12 (s, 3H), 1.37 (br s, 1H), 1.07 (d, J=6.5 Hz, 3H). LCMS: (Method A) Rf=4.830 min, (ESI) m/z: 647.1 ([M+H]⁺, 100%).

### (S)-N-(2-(4-(1-Acetyl-2-methyl-1,2,3,4-tetrahydroquinolin-6-yl)benzamido)ethyl)-2-(2-aminopyrimidin-5-yl)-7-methyl-4-morpholinothieno[3,2-d]pyrimidine-6-carboxamide [Cpd 033]

A solution of (S)-N-(2-(4-(1-acetyl-2-methyl-1,2,3,4-tetrahydroquinolin-6-yl)benzamido)ethyl)-2-chloro-7-methyl-4-morpholinothieno[3,2-d]pyrimidine-6-carboxamide (S17.i[Cpd 033]) (50 mg, 0.08 mmol), 2-aminopyrimidine-5-boronic acid (18 mg, 0.13 mmol), K₂CO₃ (35 mg, 0.25 mmol) in a mixture of DME/H₂O (4:1, 5 mL) was degassed with N₂ for 15 min. Pd(dppf)Cl₂ (12 mg, 0.02 mmol) was then added and the mixture was further degassed for another 15 min. The reaction vessel was then evacuated and backfilled with N₂ three times. The reaction mixture was heated to reflux under N₂ for 24 hr. The mixture was then diluted with water and extracted with a mixture of DCM and isopropanol (3:1) (×3). The organic extracts were combined, dried over MgSO₄ and concentrated in vacuo. The resulting residue was purified using flash column chromatography (0-8% MeOH in DCM) to give a brown solid, which was triturated with cold MeOH to give the title compound as an off-white powder (29 mg, 53%). ¹H NMR (401 MHz, DMSO) δ 9.17 (s, 2H), 8.67 (br t, J=4.8 Hz, 1H), 8.54 (br t, J=4.6 Hz, 1H), 7.95 (d, J=8.4 Hz, 2H), 7.79 (d, J=8.4 Hz, 2H), 7.61 (s, 1H), 7.57 (dd, J=8.4, 1.6 Hz, 1H), 7.46 (br s, 1H), 7.13 (s, 2H), 4.72-4.56 (m, 1H), 3.98 (app. t, 4H), 3.78 (app. t, 4H), 3.50 (s, 4H), 2.74 (dt, J=10.7, 5.1 Hz, 1H), 2.63-2.54 (m, 4H), 2.36-2.23 (m, 1H), 2.12 (s, 3H), 1.37 (br s, 1H), 1.06 (d, J=6.4 Hz, 3H). LCMS: (Method A) Rf=4.394 min, (ESI) m/z: 706.0 ([M+H]⁺, 100%). HRMS (ESI⁺) calcd for [C₃₇H₃₉N₉O₄S+H]⁺706.2918, found 706.2940.

### (R)-2-Methyl-1,2,3,4-tetrahydroquinoline (S9.4 R enantiomer)

The aqueous layer from the chiral resolution was basified to pH=14 with 10% NaOH solution and extracted with DCM (×3). The organic extracts were combined, dried over MgSO₄ and concentrated in vacuo to give the (R)-enantiomerically enriched product (5.1 g, 60% ee). This was resolved again using the same procedure mentioned above with the chiral auxiliary being the limiting reagent and (R)-enriched 2-methyl-1,2,3,4-tetrahydroquinoline being 1.5 eq. After the reaction was completed, the reaction mixture was washed with 1N HCl (×2). The aqueous washes were combined, basified to pH=14 with 10% NaOH solution and extracted with DCM (×3). The organic extracts were combined, dried over MgSO₄ and concentrated in vacuo. The residue was purified using flash column chromatography (isocratic 100% DCM) to give the title compound as a light yellow oil (2.43 g, >99.5% ee).

### (R)-1-(2-Methyl-3,4-dihydroquinolin-1(2H)-yl)ethan-1-one (S9.5 R enantiomer)

Refer to the synthesis of S2.1.

### (R)-1-(6-Bromo-2-methyl-3,4-dihydroquinolin-1(2H)-yl)ethan-1-one (S9.6 R enantiomer)

Refer to the synthesis of S2.2.

### (R)-1-(6-(4-(Aminomethyl)phenyl)-2-methyl-3,4-dihydroquinolin-1(2H)-yl)ethan-1-one (S9.12 R enantiomer)

Refer to the synthesis of S9.12.

### (R)-N-(4-(1-Acetyl-2-methyl-1,2,3,4-tetrahydroquinolin-6-yl)benzyl)-6-bromo-8-morpholinoimidazo[1,2-a]pyrazine-2-carboxamide (S17.i[Cpd 034])

A solution of (R)-1-(6-(4-(aminomethyl)phenyl)-2-methyl-3,4-dihydroquinolin-1(2H)-yl)ethan-1-one (S9.12 R enantiomer) (140 mg, 0.48 mmol), lithium(I) 6-bromo-8-morpholinoimidazo[1,2-a]pyrazine-2-carboxylate (S1.3) (206 mg, 0.62 mmol), HCTU (393 mg, 0.95 mmol), DIPEA (0.5 mL, 2.87 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with DCM and PE to give the title compound as an off-white powder (204 mg, 71%). ¹H NMR (401 MHz, CDCl₃) δ 8.04 (s, 1H), 7.64 (s, 1H), 7.60-7.54 (m, 2H), 7.51-7.39 (m, 4H), 7.37 (br s, 1H), 7.21 (br s, 1H), 4.84 (br s, 1H), 4.72 (d, J=6.3 Hz, 2H), 4.30 (s, 4H), 3.83 (app. t, 4H), 2.68 (dt, J=14.7, 5.0 Hz, 1H), 2.64-2.53 (m, 1H), 2.37 (ddt, J=10.2, 7.7, 5.1 Hz, 1H), 2.19 (s, 3H), 1.40 (br s, 1H), 1.16 (d, J=6.5 Hz, 3H). LCMS: (Method A) Rf=5.205 min, (ESI) m/z: 602.9, 604.9 ([M+H]⁺, 100%).

### (R)-N-(4-(1-Acetyl-2-methyl-1,2,3,4-tetrahydroquinolin-6-yl)benzyl)-6-(2-aminopyrim-idin-5-yl)-8-morpholinoimidazo[1,2-a]pyrazine-2-carboxamide [Cpd 034]

A solution of (R)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 034]) (100 mg, 0.17 mmol), 2-aminopyrimidine-5-boronic acid (28 mg, 0.20 mmol), K₂CO₃ (69 mg, 0.50 mmol) in a mixture of DME/H₂O (4:1, 5 mL) was degassed with N₂ for 15 min. Pd(dppf)Cl₂ (12 mg, 0.02 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-5% MeOH in DCM) to give a dark yellow solid, which was triturated with MeOH to give the title compound as an off-white powder (64 mg, 63%). Characterisation refers to the racemic form and the (S)-enantiomer. LCMS: (Method A) Rf=4.466 min, (ESI) m/z: 618.0 ([M+H]⁺, 100%). HRMS (ESI⁺) calcd for [C₃₄H₃₅N₉O₃+H]⁺618.2936, found 618.2952.

### Ethyl 6-bromo-8-(4-(tert-butoxycarbonyl)piperazin-1-yl)imidazo[1,2-a]pyrazine-2-carboxylate (S22.1)

To a solution of ethyl 6,8-dibromoimidazo[1,2-a]pyrazine-2-carboxylate (S1.1) (500 mg, 1.43 mmol) in DCM (10 mL) was added Boc-piperazine (320 mg, 1.72 mmol) and TEA (0.3 mL, 2.15 mmol). The reaction mixture was stirred at r.t for 6 hr. The mixture was then diluted with DCM and washed with 0.5 M HCl solution (×2). The organic layer was then dried over MgSO₄ and concentrated in vacuo to give the title compound as a light yellow solid (643 mg, 99%). This compound was used directly in the next step without further purification. ¹H NMR (401 MHz, CDCl₃) δ 7.97 (s, 1H), 7.58 (s, 1H), 4.42 (q, J=7.1 Hz, 2H), 4.35 (br s, 4H), 3.62-3.54 (m, 4H), 1.49 (s, 9H), 1.41 (t, J=7.1 Hz, 3H). LCMS: (Method B) Rf=3.307 min, (ESI) m/z: 398.0, 400.0 ([(M-tert-butyl)+H]⁺, 100%), 454.1, 456.1 ([(M+H]⁺, 10%).

### Lithium(I) 6-bromo-8-(4-(tert-butoxycarbonyl)piperazin-1-yl)imidazo[1,2-a]pyrazine-2-carboxylate (S22.2)

To a solution of ethyl 6-bromo-8-(4-(tert-butoxycarbonyl)piperazin-1-yl)imidazo[1,2-a]pyrazine-2-carboxylate (S22.1) (600 mg, 1.32 mmol) in a mixture of THE/water (4:1) was added LiOH·H₂O (1.2 eq, mass mg, mmol). The reaction mixture was heated to reflux for 3.5 hr. The THE was concentrated in vacuo. To the remaining aqueous residue was added acetone, the resulting precipitate was filtered to give the title compound as a pale yellow powder (540 mg, 95%). ¹H NMR (401 MHz, D₂O) δ 7.81 (s, 1H), 7.54 (s, 1H), 4.04 (br s, 4H), 3.48 (br s, 4H), 1.42 (s, 9H). LCMS: (Method C) Rf=3.707 min, (ESI) m/z: 326.1, 328.0 ([(M-Boc)+H]⁺, 100%), 370.0, 372.0 ([(M-tert-butyl)+H]⁺, 100%).

### tert-Butyl (S)-4-(2-((4-(1-acetyl-2-methyl-1,2,3,4-tetrahydroquinolin-6-yl)benzyl)car-bamoyl)-6-bromoimidazo[1,2-a]pyrazin-8-yl)piperazine-1-carboxylate (S22.3)

To a solution of (S)-1-(6-(4-(aminomethyl)phenyl)-2-methyl-3,4-dihydroquinolin-1(2H)-yl)ethan-1-one (S9.12) (200 mg, 0.68 mmol) in DMF (5 mL) was added lithium(I) 6-bromo-8-(4-(tert-butoxycarbonyl)piperazin-1-yl)imidazo[1,2-a]pyrazine-2-carboxylate (S22.2) (440 mg, 1.02 mmol), EDC.HCl (261 mg, 1.36 mmol), HOBT (184 mg, 1.36 mmol) and DIPEA (0.71 mL, 4.08 mmol). The reaction mixture was stirred at r.t for 1.5 d and then poured into a 5% Na₂CO₃ solution. The resulting precipitate was filtered and purified using flash column chromatography (0-2% MeOH in DCM) to give an oil, which was triturated with EA and PE to give the title compound as a white powder (193 mg, 40%). ¹H NMR (401 MHz, CDCl₃) δ 8.04 (s, 1H), 7.64 (s, 1H), 7.57 (d, J=8.3 Hz, 2H), 7.49 (br t, J=6.2 Hz, 1H), 7.46-7.40 (m, 3H), 7.38 (br s, 1H), 7.20 (br s, 1H), 4.83 (br s, 1H), 4.73 (d, J=6.2 Hz, 2H), 4.27 (br s, 4H), 3.63-3.50 (m, 4H), 2.69 (dt, J=14.6, 4.9 Hz, 1H), 2.63-2.51 (m, 1H), 2.37 (ddt, J=10.1, 7.6, 5.0 Hz, 1H), 2.19 (s, 3H), 1.48 (s, 9H), 1.41 (br s, 1H), 1.16 (d, J=6.5 Hz, 3H). LCMS: (Method B) Rf=3.332 min, (ESI) m/z: 602.2, 604.2 ([(M-Boc)+H]⁺, 100%).

### tert-Butyl (S)-4-(2-((4-(1-acetyl-2-methyl-1,2,3,4-tetrahydroquinolin-6-yl)benzyl)car-bamoyl)-6-(2-aminopyrimidin-5-yl)imidazo[1,2-a]pyrazin-8-yl)piperazine-1-carboxylate (S22.4)

A solution of tert-butyl (S)-4-(2-((4-(1-acetyl-2-methyl-1,2,3,4-tetrahydroquinolin-6-yl)benzyl)carbamoyl)-6-bromoimidazo[1,2-a]pyrazin-8-yl)piperazine-1-carboxylate (S22.3) (100 mg, 0.14 mmol), 2-aminopyrimidine-5-boronic acid (26 mg, 0.19 mmol), K₂CO₃ (50 mg, 0.43 mmol) in a mixture of DME/H₂O (4:1, 5 mL) was degassed with N₂ for 15 min. Pd(dppf)Cl₂ (10 mg, 0.01 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-4% MeOH in DCM) to give an oil, which was triturated with DCM and PE to give the title compound as a light beige powder (101 mg, 99%). ¹H NMR (401 MHz, CDCl₃) δ 8.81 (s, 2H), 8.15 (s, 1H), 7.84 (s, 1H), 7.62-7.52 (m, 3H), 7.46 (d, J=8.1 Hz, 2H), 7.42 (dd, J=8.2, 1.8 Hz, 1H), 7.38 (br s, 1H), 7.20 (br s, 1H), 5.40 (br s, 2H), 4.83 (br s, 1H), 4.74 (d, J=6.1 Hz, 2H), 4.31 (br s, 4H), 3.67-3.54 (m, 4H), 2.69 (dt, J=14.5, 4.8 Hz, 1H), 2.63-2.52 (m, 1H), 2.43-2.31 (m, 1H), 2.19 (s, 3H), 1.48 (s, 9H), 1.43 (br s, 1H), 1.16 (d, J=6.5 Hz, 3H). LCMS: (Method B) Rf=2.912 min, (ESI) m/z: 717.4 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-(piperazin-1-yl)imidazo[1,2-a]pyrazine-2-carboxamide hydrochloride [Cpd 035]

To a solution of tert-butyl (S)-4-(2-((4-(1-acetyl-2-methyl-1,2,3,4-tetrahydroquinolin-6-yl)benzyl)carbamoyl)-6-(2-aminopyrimidin-5-yl)imidazo[1,2-a]pyrazin-8-yl)piperazine-1-carboxylate (S22.4) (55 mg, 0.08 mmol) in dioxane (5 mL) was added 4N HCl in dioxane (5 mL). The reaction mixture was stirred at r.t for 1 hr and the solvent was then concentrated in vacuo. The residue was then triturated with MeOH and diethyl ether. The resulting gel-ish precipitate was centrifuged and the supernatant solution was decanted. The precipitate was repeatedly suspended in diethyl ether, centrifuged and the supernatant decanted. The precipitate was then suspended in ACN and lyophilised to give the title compound as an off-white powder (51 mg, 96%). ¹H NMR (401 MHz, MeOD) δ 9.04 (s, 2H), 8.52 (s, 1H), 8.35 (s, 1H), 7.62 (d, J=8.0 Hz, 2H), 7.52-7.44 (m 4H), 7.31 (br s, 1H), 4.68 (s, 6H), 3.43 (br s, 4H), 2.78-2.69 (m, 1H), 2.64-2.51 (m, 1H), 2.46-2.36 (m, 1H), 2.17 (s, 3H), 1.37 (br s, 1H). 1.14 (d, J=6.0 Hz, 3H). Tetrahydroquinaldine —N(Ac)CH(—CH₂—)(—CH₃) was not observed due to overlap with the water peak. LCMS: (Method B) Rf=2.311 min, (ESI) m/z: 617.3 ([M+H]⁺, 100%). HRMS (ESI⁺) calcd for [C₃₄H₃₆N₁₀O₂+H]⁺617.3095, found 617.3115.

### (S)-1-(2-Methyl-6-(4-((methylamino)methyl)phenyl)-3,4-dihydroquinolin-1(2H)-yl)ethan-1-one (510.2)

Step 1: To a solution of (S)-4-(1-acetyl-2-methyl-1,2,3,4-tetrahydroquinolin-6-yl)benzaldehyde (S9.7) (300 mg. 1.02 mmol) in MeOH (5 mL) was added methylamine (33% in abs. EtOH) (1 mL, 8.3 mmol). The reaction mixture was stirred at r.t o.n. The reaction mixture was then concentrated in vacuo get give the imine intermediate (S10.1). This was directly used in the next step without further purification. ¹H NMR (401 MHz, CDCl₃) δ 8.35-8.30 (m, 1H), 7.78 (d with fine splitting, J=8.3 Hz, 2H), 7.64 (d with fine splitting, J=8.3 Hz, 2H), 7.47 (dd, J=8.1, 1.9 Hz, 1H), 7.43 (sd, J=1.6 Hz, 1H), 7.23 (br s, 1H), 4.85 (br s, 1H), 3.55 (d, J=1.6 Hz, 3H), 2.70 (dt, J=14.7, 5.1 Hz, 1H), 2.65-2.52 (m, 1H), 2.38 (ddt, J=12.8, 7.5, 5.0 Hz, 1H), 2.20 (s, 3H), 1.43 (br s, 1H), 1.16 (d, J=6.5 Hz, 3H). Step 2: The imine intermediate was re-dissolved in MeOH (5 mL) and cooled to 0° C. NaBH₄ (116 mg, 3.07 mmol) was added in portions. The reaction mixture was warmed to r.t and stirred at this temperature o.n. To the reaction mixture was added water (1 mL) and the MeOH concentrated in vacuo. To the aqueous residue was added aq. 10% NaOH and extracted with DCM (×3). The organic extracts were combined, dried over MgSO₄ and concentrated in vacuo to give the title product as a light yel low oil (308 mg, 98%). This product was used directly in the next step without further purifi cation. ¹H NMR (401 MHz, CDCl₃) δ 7.56 (d, J=8.3 Hz, 2H), 7.43-7.37 (m, 4H), 7.19 (br s, 1H), 4.84 (s, 1H), 3.82 (s, 2H), 2.68 (dt, J=9.5, 4.5 Hz, 1H), 2.57 (ddd, J=10.0, 7.6, 3.9 Hz, 1H), 2.50 (s, 3H), 2.37 (ddt, J=10.3, 7.0, 5.2 Hz, 1H), 2.19 (s, 3H), 1.39 (br s, 1H), 1.16 (d, J=6.5 Hz, 3H). LCMS: (Method C) Rf=2.979 min, (ESI) m/z: 309.2 ([M+H]⁺, 30%), 278.2 ([(M-NHCH₃)+H]⁺, 100%).

### 6-Bromo-N-methyl-N-(2-(methylamino)ethyl)-8-morpholinoimidazo[1,2-a]pyrazine-2-carboxamide (513.2)

To a solution of ethyl 6-bromo-8-morpholinoimidazo[1,2-a]pyrazine-2-carboxylate (S1.2) (400 mg, 1.13 mmol) in abs. EtOH (8 mL) was added N,N′-dimethylethylenediamine (3 mL). The reaction mixture was heated to reflux for 48 hr. The solvent was then concentrated in vacuo. The crude material was suspended in 10% (w/w) NaOH solution and the precipitate was filtered, washed with water and dried on air to give the title compound as a white powder (365 mg, purity: ˜90%). ¹H NMR (401 MHz, CDCl₃) δ 7.97 (s, 1H), 7.60 (s, 1H), 4.32 (br s, 4H), 3.89-3.80 (app. t, 4H), 3.49, 3.14 (s,s, 3H), 2.89 (t, J=6.7 Hz, 2H), 2.48, 2.40 (s,s, 3H). LCMS: (Method C) Rf=2.972 min, (ESI) m/z: 397.1, 399.1 ([M+H]⁺, 100%).

### (S)-N-(2-(4-(1-Acetyl-2-methyl-1,2,3,4-tetrahydroquinolin-6-yl)-N-methylbenzamido)ethyl)-6-bromo-N-methyl-8-morpholinoimidazo[1,2-a]pyrazine-2-carboxamide (S17.i[Cpd 036])

To a solution of 6-bromo-N-methyl-N-(2-(methylamino)ethyl)-8-morpholinoimidazo[1,2-a]pyrazine-2-carboxamide (S13.2) (purity: ˜90%) (110 mg, 0.28 mmol) in DMF (5 mL) was added (S)-4-(1-acetyl-2-methyl-1,2,3,4-tetrahydroquinolin-6-yl)benzoic acid (S9.8) (111 mg, 0.36 mmol), EDC.HCl (80 mg, 0.42 mmol), HOBT (57 mg, 0.42 mmol) and DIPEA (0.26 mL, 1.50 mmol). The reaction mixture was stirred at r.t for 1.5 d. The reaction mixture was then poured into 5% Na₂CO₃ solution, the resulting precipitate was filtered and purified using flash column chromatography (0-4% MeOH in DCM) to give a semi-solid, which was co-evaporated with DCM and PE to give the title compound as an off-white powder (173 mg, 91%). LCMS: (Method C) Rf=3.583 min, (ESI) m/z: 688.2, 690.2 ([M+H]⁺, 100%).

### (S)-N-(2-(4-(1-Acetyl-2-methyl-1,2,3,4-tetrahydroquinolin-6-yl)-N-methylbenzamido)ethyl)-6-(2-aminopyrimidin-5-yl)-N-methyl-8-morpholinoimidazo[1,2-a]pyrazine-2-carboxamide [Cpd 036]

A solution of (S)-N-(2-(4-(1-acetyl-2-methyl-1,2,3,4-tetrahydroquinolin-6-yl)-N-methylbenzamido)ethyl)-6-bromo-N-methyl-8-morpholinoimidazo[1,2-a]pyrazine-2-carboxamide (S17.i[Cpd 036]) (140 mg, 0.20 mmol), 2-aminopyrimidine-5-boronic acid (34 mg, 0.24 mmol), K₂CO₃ (84 mg, 0.61 mmol) in a mixture of DME/H₂O (4:1, 5 mL) was degassed with N₂ for 15 min. Pd(dppf)Cl₂ (15 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-8% MeOH in DCM) to give a yellow solid, which was triturated with a cold mixture of MeOH and diethyl ether (1:1) to give the title compound as a white powder (106 mg, 74%). LCMS: (Method B) Rf=2.453 min, (ESI) m/z: 703.3 ([M+H]⁺, 100%). HRMS (ESI⁺) calcd for [C₃₈H₄₂N₁₀O₄+H]⁺703.3463, found 703.3479.

### Ethyl (S)-4-(1-acetyl-2-methyl-1,2,3,4-tetrahydroquinolin-6-yl)benzoate (59.16)

A solution of (S)-1-(6-bromo-2-methyl-3,4-dihydroquinolin-1(2H)-yl)ethan-1-one (S9.6) (500 mg, 1.86 mmol), 4-ethoxycarbonylphenylboronic acid (520 mg, 2.68 mmol), K₂CO₃ (928 mg, 6.71 mmol) in a mixture of DME/H₂O (4:1, 10 mL) was degassed with N₂ for 15 min. Pd(dppf)Cl₂ (164 mg, 0.22 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-50% EA in PE) to give the title compound as a white powder (685 mg, 91%). ¹H NMR (400 MHz, CDCl₃) δ 8.13 (d, J=8.6 Hz, 2H), 7.66 (d, J=8.6 Hz 2H), 7.46 (dd, J=8.2, 2.0 Hz, 1H), 7.44 (s, 1H), 7.24 (br s, 1H), 4.86 (br s, 1H), 4.42 (q, J=7.1 Hz, 2H), 2.78-2.67 (m, 1H), 2.66-2.54 (m, 1H), 2.45-2.31 (m, 1H), 2.20 (s, 3H), 1.42 (app. t, 4H), 1.17 (d, J=6.5 Hz, 3H). LCMS: (Method B) Rf=4.057 min, (ESI) m/z: 338.2 ([M+H]⁺, 100%).

### (S)-N-(4-(1-Acetyl-2-methyl-1,2,3,4-tetrahydroquinolin-6-yl)benzyl)-6-bromo-N-methyl-8-morpholinoimidazo[1,2-a]pyrazine-2-carboxamide (S17.i[Cpd 037])

To a solution of (S)-1-(2-methyl-6-(4-((methylamino)methyl)phenyl)-3,4-dihydroquin-olin-1(2H)-yl)ethan-1-one (S10.2) (100 mg, 0.32 mmol) in DMF (5 mL) was added lithium(I) 6-bromo-8-morpholinoimidazo[1,2-a]pyrazine-2-carboxylate (S1.3) (111 mg, 0.39 mmol), EDC.HCl (93 mg, 0.49 mmol), HOBT (66 mg, 0.49 mmol) and DIPEA (0.34 mL, 1.94 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give a semi-solid, which was co-evaporated with DCM and PE to give the title compound as a white powder (144 mg, 72%). ¹H NMR (401 MHz, CDCl₃) δ 8.05 (br s, 1H), 7.65-7.53 (m, 3H), 7.45-7.35 (m, 4H), 7.20 (br s, 1H), 5.18 (s, 1.5H, methylene proton), 4.84 (br s, 1H, THQ chiral centre —CH— proton), 4.81 (s, 0.5H, methylene proton), 4.33 (br s, 1H, morpholine proton), 4.02 (br s, 3H, morpholine protons), 3.87-3.78 (m, 1H, morpholine proton), 3.49 (br s, 3H, morpholine proton), 3.46 (s, 0.75H, —N—CH₃ pro ton), 3.12 (s, 2.25H, —N—CH₃ proton), 2.69 (dt, J=14.1, 4.5 Hz, 1H), 2.64-2.51 (m, 1H), 2.44-2.31 (m, 1H), 2.20 (s, 3H), 1.41 (s, 1H), 1.17 (d, J=6.4 Hz, 3H). LCMS: (Method C) Rf=4.024 min, (ESI) m/z: 617.2, 619.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-N-methyl-8-morpholinoimidazo[1,2-a]pyrazine-2-carboxamide [Cpd 037]

A solution of (S)-N-(4-(1-acetyl-2-methyl-1,2,3,4-tetrahydroquinolin-6-yl)benzyl)-6-bromo-N-methyl-8-morpholinoimidazo[1,2-a]pyrazine-2-carboxamide (S17.i[Cpd 037]) (100 mg, 0.16 mmol), 2-aminopyrimidine-5-boronic acid (27 mg, 0.19 mmol), K₂CO₃ (68 mg, 0.49 mmol) in a mixture of DME/H₂O (4:1, 5 mL) was degassed with N₂ for 15 min. Pd(dppf)Cl₂ (12 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a light brown solid, which was triturated with MeOH to give the title compound as a white powder (85 mg, 83%). ¹H NMR (401 MHz, CDCl₃) δ 8.83 (s, 0.5H, pyrimidine —CH proton), 8.80 (s, 1.5H, pyrimidine —CH proton), 8.17 (s, 1H), 7.84 (s, 0.25H, imidazole pyrazine —CH proton), 7.82 (s, 0.75H, imidazole pyrazine —CH proton), 7.60 (d, J=8.1 Hz, 2H), 7.49-7.38 (m, 4H), 7.23 (br s, 1H), 5.25 (s, 1.5H, linker methylene protons), 5.20 (s, 2H, pyrimidine amino protons), 4.87 (br s, 1H, THQ chiral centre —CH— proton), 4.85 (s, 0.5H, linker methylene protons), 4.40 (br s, 1H, morpholine protons), 4.10 (br s, 3H, morpholine pro tons), 3.89 (br s, 1H, morpholine protons), 3.57 (s, 3H, morpholine protons), 3.52 (s, 0.75H, —N—CH₃ proton), 3.15 (s, 2.25H, —N—CH₃ proton), 2.77-2.67 (m, 1H), 2.66-2.55 (m, 1H), 2.47-2.34 (m, 1H), 2.23 (s, 3H), 1.44 (br s, 1H), 1.19 (d, J=6.4 Hz, 3H). LCMS: (Method C) Rf=3.504 min, (ESI) m/z: 632.3 ([M+H]⁺, 100%). HRMS (ESI⁺) calcd for [C₃₅H₃₇N₉O₃+H]⁺632.3092, found 632.3106.

### (S)-N-(4-(1-Acetyl-2-methyl-1,2,3,4-tetrahydroquinolin-6-yl)benzyl)-2-chloro-N-methyl-4-morpholinothieno[3,2-d]pyrimidine-6-carboxamide (S17.i[Cpd 038])

To a solution of (S)-1-(2-methyl-6-(4-((methylamino)methyl)phenyl)-3,4-dihydroquin-olin-1(2H)-yl)ethan-1-one (S10.2) (100 mg, 0.32 mmol) in DMF (5 mL) was added 2-chloro-4-morpholinothieno[3,2-d]pyrimidine-6-carboxylic acid (S15.10) (117 mg, 0.39 mmol), EDC.HCl (93 mg, 0.49 mmol), HOBT (66 mg, 0.49 mmol) and DIPEA (0.34 mL, 1.94 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil, which was triturated with EA/PE to give the title compound as a white powder (105 mg, 55%). ¹H NMR (401 MHz, CDCl₃) δ 7.60 (d, J=7.5 Hz, 2H), 7.48-7.35 (m, 4H), 7.33-7.27 (m, 1H), 7.22 (br s, 1H), 4.83 (s, 3H, chiral centre —CH— proton overlapped with the linker methylene protons), 4.09-3.95 (m, 4H), 3.91-3.78 (m, 4H), 3.19 (br d, J=15.5 Hz, 3H, N-methyl protons showed splitting), 2.76-2.65 (m, 1H), 2.64-2.52 (m, 1H), 2.44-2.32 (m, 1H), 2.20 (s, 3H), 1.42 (br s, 1H), 1.17 (d, J=6.4 Hz, 3H). LCMS: (Method C) Rf=3.929 min, (ESI) m/z: 590.2 ([M+H]⁺, 100%), 592.2 ([M+H]⁺, 30%).

### (S)-N-(4-(1-Acetyl-2-methyl-1,2,3,4-tetrahydroquinolin-6-yl)benzyl)-2-(2-aminopyrim-idin-5-yl)-N-methyl-4-morpholinothieno[3,2-d]pyrimidine-6-carboxamide [Cpd 038]

A solution of (S)-N-(4-(1-acetyl-2-methyl-1,2,3,4-tetrahydroquinolin-6-yl)benzyl)-2-chloro-N-methyl-4-morpholinothieno[3,2-d]pyrimidine-6-carboxamide (S17.i[Cpd 038]) (80 mg, 0.14 mmol), 2-aminopyrimidine-5-boronic acid (29 mg, 0.21 mmol), K₂CO₃ (56 mg, 0.41 mmol) in a mixture of DME/H₂O (4:1, 5 mL) was degassed with N₂ for 15 min. Pd(dppf)Cl₂ (20 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×3). The organic extracts were combined, dried over MgSO₄ and concentrated in vacuo. The resulting residue was purified using flash column chromatography (0-5% MeOH in DCM) to give a yellow solid, which was triturated with MeOH to give the title compound as a white powder (72 mg, 82%). ¹H NMR (401 MHz, CDCl₃) δ 9.25 (br s, 2H), 7.65-7.31 (m, 7H), 7.22 (s, 1H), 5.31 (s, 2H), 4.85 (br s, 3H, linker methylene protons overlapped with the chiral centre —CH— proton), 4.07 (br s, 4H), 3.88 (app. t, 4H), 3.21 (br d, J=43.9 Hz, 3H, N-methyl protons showed splitting, large coupling constant), 2.70 (dt, J=14.3, 4.7 Hz, 1H), 2.64-2.52 (m, 1H), 2.45-2.30 (m, 1H), 2.20 (s, 3H), 1.41 (br s, 1H), 1.17 (d, J=6.5 Hz, 3H). LCMS: (Method C) Rf=3.503 min, (ESI) m/z: 649.3 ([M+H]⁺, 100%). HRMS (ESI⁺) calcd for [C₃₅H₃₆N₈O₃S+H]⁺649.2704, found 649.2720.

### (S)-N-(4-(1-Acetyl-2-methyl-1,2,3,4-tetrahydroquinolin-6-yl)benzyl)-2-chloro-N,7-dimethyl-4-morpholinothieno[3,2-d]pyrimidine-6-carboxamide (S17.i[Cpd 039])

A solution of (S)-1-(2-methyl-6-(4-((methylamino)methyl)phenyl)-3,4-dihydroquino-lin-1(2H)-yl)ethan-1-one (S10.2) (100 mg, 0.32 mmol), lithium(I) 2-chloro-7-methyl-4-morpholinothieno[3,2-d]pyrimidine-6-carboxylate (S16.5) (155 mg, 0.48 mmol), HCTU (268 mg, 0.65 mmol), DIPEA (0.34 mL, 1.94 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was triturated with EA and PE to give the title compound as an off-white powder (90 mg, 46%). LCMS: (Method C) Rf=4.012 min, (ESI) m/z: 604.2 ([M+H]⁺, 100%), 606.2 ([M+H]⁺, 30%).

### (S)-N-(4-(1-Acetyl-2-methyl-1,2,3,4-tetrahydroquinolin-6-yl)benzyl)-2-(2-aminopyrim-idin-5-yl)-N,7-dimethyl-4-morpholinothieno[3,2-d]pyrimidine-6-carboxamide [Cpd 039]

A solution of (S)-N-(4-(1-acetyl-2-methyl-1,2,3,4-tetrahydroquinolin-6-yl)benzyl)-2-chloro-N,7-dimethyl-4-morpholinothieno[3,2-d]pyrimidine-6-carboxamide (S17.i[Cpd 039]) (90 mg, 0.15 mmol), 2-aminopyrimidine-5-boronic acid (31 mg, 0.22 mmol), K₂CO₃ (62 mg, 0.45 mmol) in a mixture of DME/H₂O (4:1, 5 mL) was degassed with N₂ for 15 min. Pd(dppf)Cl₂ (22 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×3). The organic extracts were combined, dried over MgSO₄ and concentrated in vacuo. The resulting residue was purified using flash column chromatography (0-5% MeOH in DCM) to give a yellow solid, which was triturated with MeOH to give the title compound as an off-white powder (80 mg, 37% over two steps). ¹H NMR (401 MHz, CDCl₃) δ 9.32 (s, 2H), 7.61-7.37 (m, 6H), 7.22 (br s, 1H), 5.30 (s, 2H), 4.83 (br s, 2H), 4.63 (br s, 1H), 4.03 (br s, 4H), 3.94-3.79 (m, 4H), 3.03 (br d, J=45.9 Hz, 3H, N-methyl protons showed splitting, large coupling constant), 2.74-2.65 (m, 1H), 2.64-2.55 (m, 1H), 2.48 (br s, 3H), 2.42-2.32 (m, 1H), 2.20 (s, 3H), 1.41 (br s, 1H), 1.16 (d, J=6.5 Hz, 3H). LCMS: (Method C) Rf=3.646 min, (ESI) m/z: 663.3 ([M+H]⁺, 100%). HRMS (ESI⁺) calcd for [C₃₆H₃₈N₈O₃S+H]⁺663.2860, found 663.2874.

### N-(3-Aminopropyl)-2-chloro-4-morpholinothieno[3,2-d]pyrimidine-6-carboxamide (S15.13)

To a solution of methyl 2-chloro-4-morpholinothieno[3,2-d]pyrimidine-6-carboxylate (S15.11) (180 mg, 0.57 mmol) in Abs. EtOH (5 mL) was added 1,3-diaminopropane (2 mL). The reaction mixture was stirred at r.t for 1.5 d. The solvent was then concentrated in vacuo. The residue was re-dissolved in DCM and washed with 10% NaOH solution (×2). The organic was dried over MgSO₄, concentrated in vacuo to give an oil, which was co-evaporated with DCM and PE, followed by diethyl ether trituration to give the title compound as an off-white powder (164 mg, 80%). ¹H NMR (401 MHz, CDCl₃) δ 9.23 (br s, 1H), 7.66 (s, 1H), 4.01 (app. t, 4H), 3.88-3.76 (m, 6H), 3.63 (br s, 2H), 3.13-3.02 (m, 2H), 1.90-1.76 (m, 2H). LCMS: (Method C) Rf=2.930 min, (ESI) m/z: 356.1 ([M+H]⁺, 100%).

### (S)-4-(1-Acetyl-2-methyl-1,2,3,4-tetrahydroquinolin-6-yl)-N-(3-aminopropyl)benzamide (S11.1)

A solution of ethyl (S)-4-(1-acetyl-2-methyl-1,2,3,4-tetrahydroquinolin-6-yl)benzoate (S9.16) (200 mg, 0.59 mmol) and 1,3-diaminopropane (2 mL) in Abs. EtOH (5 mL) was heated to reflux in a sealed tube for 2 d. The solvent was then concentrated in vacuo. The residue was re-dissolved in DCM and washed with 10% NaOH solution (×2). The organic was dried over MgSO₄, concentrated in vacuo to give the title compound as light yellow solid (209 mg, 96%).

¹H NMR (401 MHz, CDCl₃) δ 7.90-7.85 (m, 2H), 7.83 (br t, J=6.9 Hz, 1H), 7.65-7.61 (m, 2H), 7.45 (dd, J=8.1, 1.9 Hz, 1H), 7.41 (br s, 1H), 7.22 (br s, 1H), 4.83 (br s, 1H), 3.62 (dt, J=6.1, 5.4 Hz, 2H), 2.94 (app. t, 2H), 2.70 (dt, J=9.6, 4.5 Hz, 1H), 2.65-2.53 (m, 1H), 2.44-2.30 (m, 1H), 2.20 (s, 3H), 1.80-1.72 (m, 2H), 1.41 (br s, 1H), 1.16 (d, J=6.5 Hz, 3H). LCMS: (Method C) Rf=2.944 min, (ESI) m/z: 366.3 ([M+H]⁺, 100%).

### (S)-N-(3-(4-(1-Acetyl-2-methyl-1,2,3,4-tetrahydroquinolin-6-yl)benzamido)propyl)-2-chloro-4-morpholinothieno[3,2-d]pyrimidine-6-carboxamide (S17.i[Cpd 040])

A solution of N-(3-aminopropyl)-2-chloro-4-morpholinothieno[3,2-d]pyrimidine-6-carboxamide (S15.13) (120 mg, 0.34 mmol), (S)-4-(1-acetyl-2-methyl-1,2,3,4-tetrahydroquin-olin-6-yl)benzoic acid (S9.8) (125 mg, 0.40 mmol), HCTU (279 mg, 0.67 mmol), DIPEA (0.35 mL, 2.03 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give an oil, which was co-evaporated with DCM and PE to give the title compound as a light yellow solid (173 mg, 79%). ¹H NMR (401 MHz, CDCl₃) δ 8.23 (br t, J=6.2 Hz, 1H), 7.93-7.86 (m, 3H), 7.66 (d, J=8.5 Hz, 2H), 7.45 (dd, J=8.2, 2.0 Hz, 1H), 7.42 (s, 1H), 7.21 (br s, 1H), 6.86 (t, J=6.6 Hz, 1H), 4.84 (br s, 1H), 4.02 (app. t, 4H), 3.84 (app. t, 4H), 3.69-3.51 (m, J=22.8, 11.8, 6.3 Hz, 4H), 2.72 (dt, J=14.7, 4.9 Hz, 1H), 2.67-2.55 (m, 1H), 2.45-2.32 (m, 1H), 2.21 (s, 3H), 1.94-1.83 (m, 2H), 1.42 (br s, 1H), 1.17 (d, J=6.5 Hz, 3H). LCMS: (Method C) Rf=3.529 min, (ESI) m/z: 647.2 ([M+H]⁺, 100%), 649.2 ([M+H]⁺, 30%).

### (S)-N-(3-(4-(1-Acetyl-2-methyl-1,2,3,4-tetrahydroquinolin-6-yl)benzamido)propyl)-2-(2-aminopyrimidin-5-yl)-4-morpholinothieno[3,2-d]pyrimidine-6-carboxamide [Cpd 040]

A solution of (S)-N-(3-(4-(1-acetyl-2-methyl-1,2,3,4-tetrahydroquinolin-6-yl)benzamido)propyl)-2-chloro-4-morpholinothieno[3,2-d]pyrimidine-6-carboxamide (S17.i[Cpd 040]) (140 mg, 0.22 mmol), 2-aminopyrimidine-5-boronic acid (46 mg, 0.33 mmol), K₂CO₃ (92 mg, 0.67 mmol) in a mixture of DME/H₂O (4:1, 5 mL) was degassed with N₂ for 15 min. Pd(dppf)Cl₂ (32 mg, 0.04 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-8% MeOH in DCM) to give a yellow solid, which was triturated with MeOH to give the title compound as a beige powder (119 mg, 78%). ¹H NMR (401 MHz, CDCl₃) δ 9.28 (s, 2H), 7.99 (br t, J=6.2 Hz, 1H), 7.96 (s, 1H), 7.93 (d, J=8.4 Hz, 2H), 7.67 (d, J=8.4 Hz, 2H), 7.45 (dd, J=8.2, 2.0 Hz, 1H), 7.41 (s, 1H), 7.24 (br s, 1H), 6.91 (br t, J=6.4 Hz, 1H), 5.32 (s, 2H), 4.83 (br s, 1H), 4.06 (app. t, 4H), 3.88 (app. t, 4H), 3.71-3.53 (m, 4H), 2.71 (dt, J=9.7, 5.1 Hz, 1H), 2.65-2.54 (m, 1H), 2.43-2.32 (m, 1H), 2.20 (s, 3H), 1.95-1.83 (m, 2H), 1.43 (br s, 1H), 1.17 (d, J=6.5 Hz, 3H). LCMS: (Method C) Rf=3.178 min, (ESI) m/z: 706.3 ([M+H]⁺, 100%). HRMS (ESI⁺) calcd for [C₃₇H₃₉N₉O₄S+H]⁺706.2918, found 706.2936.

### (S)-N-(3-(4-(1-Acetyl-2-methyl-1,2,3,4-tetrahydroquinolin-6-yl)benzamido)propyl)-2-chloro-7-methyl-4-morpholinothieno[3,2-d]pyrimidine-6-carboxamide (S17.i[Cpd 041])

A solution of (S)-4-(1-acetyl-2-methyl-1,2,3,4-tetrahydroquinolin-6-yl)-N-(3-ami-nopropyl)benzamide (S11.1) (90 mg, 0.25 mmol), lithium(I) 2-chloro-7-methyl-4-morpholinothieno[3,2-d]pyrimidine-6-carboxylate (S16.5) (102 mg, 0.32 mmol), PyClock (273 mg, 0.49 mmol), DIPEA (0.27 mL, 1.55 mmol) in DMF (5 mL) was stirred at r.t for 24 hr. The reaction mixture was then poured into 5% Na₂CO₃ solution, the resulting precipitate was fil tered and purified using flash column chromatography (0-3% MeOH in DCM) to give an oil, which was triturated with DCM and PE to give the title compound as a white powder (108 mg, 67%). ¹H NMR (401 MHz, CDCl₃) δ 7.87 (d, J=8.5 Hz, 2H), 7.64 (d, J=8.5 Hz, 2H), 7.45 (dd, J=8.2, 2.0 Hz, 1H), 7.41 (br s, 1H), 7.29 (br t, J=6.6 Hz, 1H), 7.24 (br s, 1H), 6.91 (br t, J=6.5 Hz, 1H), 4.83 (br s, 1H), 4.00 (app. t, 4H), 3.82 (app. t, 4H), 3.69-3.55 (m, 4H), 2.77-2.67 (m, 4H), 2.66-2.54 (m, 1H), 2.43-2.33 (m, 1H), 2.20 (s, 3H), 1.95-1.84 (m, 2H), 1.17 (d, J=6.5 Hz, 3H). LCMS: (Method C) Rf=3.647 min, (ESI) m/z: 661.3 ([M+H]⁺, 100%), 663.3 ([M+H]⁺, 30%).

### (S)-N-(3-(4-(1-Acetyl-2-methyl-1,2,3,4-tetrahydroquinolin-6-yl)benzamido)propyl)-2-(2-aminopyrimidin-5-yl)-7-methyl-4-morpholinothieno[3,2-d]pyrimidine-6-carboxamide [Cpd 041]

A solution of (S)-N-(3-(4-(1-acetyl-2-methyl-1,2,3,4-tetrahydroquinolin-6-yl)benzamido)propyl)-2-chloro-7-methyl-4-morpholinothieno[3,2-d]pyrimidine-6-carboxamide (S17.i[Cpd 041]) (75 mg, 0.11 mmol), 2-aminopyrimidine-5-boronic acid (24 mg, 0.17 mmol), K₂CO₃ (47 mg, 0.34 mmol) in a mixture of DME/H₂O (4:1, 5 mL) was degassed with N₂ for 15 min. Pd(dppf)Cl₂ (17 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with a mixture of DCM/isopropanol (3:1, ×2). The organic extracts were combined, dried over MgSO₄ and concentrated in vacuo. The resulting residue was purified using flash column chromatography (0-8% MeOH in DCM) to give a yellow solid, which was triturated with MeOH to give the title compound as an off-white powder (50 mg, 61%). ¹H NMR (401 MHz, DMSO) δ 9.17 (s, 2H), 8.57 (br t, J=5.6 Hz, 1H), 8.46 (br t, J=5.7 Hz, 1H), 7.91 (d, J=8.5 Hz, 2H), 7.75 (d, J=8.5 Hz, 2H), 7.61-7.51 (m, 2H), 7.44 (br s, 1H), 7.13 (s, 2H), 4.71-4.55 (m, 1H), 3.97 (app. t, 4H), 3.77 (app. t, 4H), 2.73 (dt, J=15.2, 5.4 Hz, 1H), 2.60 (s, 3H), 2.57-2.53 (m, 1H), 2.35-2.23 (m, 1H), 2.12 (s, 3H), 1.90-1.76 (m, 2H), 1.36 (br s, 1H), 1.06 (d, J=6.4 Hz, 3H). LCMS: (Method C) Rf=3.303 min, (ESI) m/z: 720.3 ([M+H]⁺, 100%). HRMS (ESI⁺) calcd for [C₃₈H₄₁N₉O₄S+H]⁺720.3075, found 720.3092.

### (S)-1-(6-(4-(2-Aminoethyl)phenyl)-2-methyl-3,4-dihydroquinolin-1(2H)-yl)ethan-1-one (S9.17)

A solution of (S)-1-(6-bromo-2-methyl-3,4-dihydroquinolin-1(2H)-yl)ethan-1-one (S9.6) (600 mg, 2.24 mmol), 2-(4-(4,4,5,5-tetramethyl-1,3,2-dioxaborolan-2-yl)phenyl)ethan-1-amine (660 mg, 2.67 mmol), K₂CO₃ (930 mg, 6.73 mmol) in a mixture of DME/H₂O (4:1, 10 mL) was degassed with N₂ for 15 min. Pd(dppf)Cl₂ (164 mg, 0.22 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-5% MeOH in DCM+1% 7N NH₃ in MeOH) to give the title compound as a yellow oil (594 mg, 86%). ¹H NMR (401 MHz, Chloroform-d) δ 7.52 (d, J=8.1 Hz, 2H), 7.41 (dd, J=8.2, 2.2 Hz, 1H), 7.37 (sd, J=2.1 Hz, 1H), 7.28 (d, J=8.1 Hz, 2H), 7.19 (br s, 1H), 4.84 (br s, 1H), 3.03 (t, J=6.9 Hz, 2H), 2.82 (t, J=6.9 Hz, 2H), 2.68 (dt, J=14.7, 5.0 Hz, 1H), 2.63-2.51 (m, 1H), 2.37 (ddt, J=12.8, 7.5, 5.0 Hz, 1H), 2.19 (s, 3H), 1.40 (br s, 1H), 1.16 (d, J=6.5 Hz, 3H). LCMS: (Method B) Rf=2.193 min, (ESI) m/z: 309.2 ([M+H]⁺, 100%).

### 2-Chloro-7-methyl-4-morpholinothieno[3,2-d]pyrimidine-6-carbaldehyde (516.7)

A suspension of 4-(2-chloro-7-methylthieno[3,2-d]pyrimidin-4-yl)morpholine (S16.3) (3.0 g, 11.1 mmol) in dry THE (30 mL) was evacuated and backfilled with nitrogen three times. The suspension was cooled to −78° C. and stirred for 10 min. To this suspension was then dropwise added n-BuLi (2.5 M in hexanes) (5.35 mL, 13.38 mmol) and the resulting mixture was stirred at −78° C. for 1 hr. To the reaction mixture was then dropwise added dry DMF (1.75 mL, 22.62 mmol) and the reaction mixture was stirred at this temperature for 2 hr then warmed up to r.t and stirred at r.t for an additional 2 hr. The reaction mixture was then poured onto a mixture of ice/HCl solution (0.25 M). The resulting mixture was stirred at 0° C. for 15 min, filtered, washed with water and dried on air to give the title compound as a bright yellow powder (2.85 g, 86%). This product was used in the next step without further purification. ¹H NMR (401 MHz, Chloroform-d) δ 10.38 (s, 1H), 4.08-4.00 (m, 4H), 3.88-3.81 (m, 4H), 2.76 (s, 3H). LCMS: (Method B) Rf=3.590 min, (ESI) m/z: 298.0 ([M+H]⁺, 100%).

### (S)-1-(6-(4-((((2-Chloro-4-morpholinothieno[3,2-d]pyrimidin-6-yl)methyl)(methyl)amino)methyl)phenyl)-2-methyl-3,4-dihydroquinolin-1(2H)-yl)ethan-1-one (S19.1)

A solution of (S)-1-(2-methyl-6-(4-((methylamino)methyl)phenyl)-3,4-dihydroquino-lin-1(2H)-yl)ethan-1-one (510.2) (150 mg, 0.49 mmol), 2-chloro-4-morpholinothieno[3,2-d]pyrimidine-6-carbaldehyde (515.4) (276 mg, 0.97 mmol) and glacial acetic acid (0.28 mL, 4.86 mmol) in dry DCE (15 mL) was stirred at r.t for 15 min. To this mixture was then added sodium triacetoxyborohydride (309 mg, 1.46 mmol). The reaction vessel was then evacuated and backfilled with N₂ three times. The reaction mixture was heated to reflux under N₂ for 3 hr. After cooling, the reaction mixture was filtered through celite and rinsed with DCM. The organic filtrate was washed with 10% NaOH solution, dried over MgSO₄ and the solvent concentrated in vacuo. The residue was then purified using flash column chromatography (0-3% MeOH in DCM) to give an oil which was co-evaporated with DCM and PE to give the title compound as a pale yellow powder (175 mg, 63%). ¹H NMR (401 MHz, Chloroform-d) δ 7.57 (d, J=8.1 Hz, 2H), 7.47-7.40 (m, 3H), 7.39 (s, 1H), 7.20 (br s, 2H), 4.84 (br s, 1H), 4.04-3.97 (m, 4H), 3.89-3.80 (m, 6H), 3.68 (br s, 2H), 2.69 (dt, J=14.7, 5.1 Hz, 1H), 2.58 (ddd, J=15.1, 10.8, 5.1 Hz, 1H), 2.44-2.29 (m, 4H), 2.19 (s, 3H), 1.41 (br s, 1H), 1.16 (d, J=6.5 Hz, 3H). LCMS: (Method B) Rf=2.556 min, (ESI) m/z: 576.2 ([M+H]⁺, 100%).

### (S)-1-(6-(4-((((2-Chloro-7-methyl-4-morpholinothieno[3,2-d]pyrimidin-6-yl)methyl)(methyl)amino)methyl)phenyl)-2-methyl-3,4-dihydroquinolin-1(2H)-yl)ethan-1-one (S19.2)

A solution of (S)-1-(2-methyl-6-(4-((methylamino)methyl)phenyl)-3,4-dihydroquino-lin-1(2H)-yl)ethan-1-one (510.2) (150 mg, 0.49 mmol), 2-chloro-7-methyl-4-morpholinothieno[3,2-d]pyrimidine-6-carbaldehyde (S16.7) (290 mg, 0.97 mmol) and glacial acetic acid (0.28 mL, 4.86 mmol) in dry DCE (15 mL) was stirred at r.t for 15 min. To this mixture was then added sodium triacetoxyborohydride (309 mg, 1.46 mmol). The reaction vessel was then evacuated and backfilled with N₂ three times. The reaction mixture was heated to reflux under N₂ for 3 hr. After cooling, the reaction mixture was filtered through celite and rinsed with DCM. The organic filtrate was washed with 10% NaOH solution, dried over MgSO₄ and the solvent concentrated in vacuo. The residue was then purified using flash column chromatography (0-3% MeOH in DCM) to give an oil which was co-evaporated with DCM and PE to give the title compound as an off-white powder (217 mg, 76%). ¹H NMR (401 MHz, Chloro form-d) δ 7.56 (d with fine splitting, J=8.2 Hz, 2H), 7.48-7.40 (m, 3H), 7.38 (sd, J=2.1 Hz, 1H), 7.19 (br s, 1H), 4.84 (br s, 1H), 4.03-3.98 (m, 4H), 3.88-3.83 (m, 4H), 3.79 (s, 2H), 3.67 (s, 2H), 2.68 (dt, J=14.7, 5.1 Hz, 1H), 2.63-2.52 (m, 1H), 2.43-2.36 (m, 1H), 2.35 (s, 3H), 2.33 (s, 3H), 2.19 (s, 3H), 1.40 (br s, 1H), 1.16 (d, J=6.5 Hz, 3H). LCMS: (Method B) Rf=2.683 min, (ESI) m/z: 590.2 ([M+H]⁺, 100%).

### (S)-1-(6-(4-((((2-(2-Aminopyrimidin-5-yl)-4-morpholinothieno[3,2-d]pyrimidin-6-yl)methyl)(methyl)amino)methyl)phenyl)-2-methyl-3,4-dihydroquinolin-1(2H)-yl)ethan-1-one [Cpd 042]

A solution of (S)-1-(6-(4-((((2-chloro-4-morpholinothieno[3,2-d]pyrimidin-6-yl)methyl)(methyl)amino)methyl)phenyl)-2-methyl-3,4-dihydroquinolin-1(2H)-yl)ethan-1-one (S19.1) (100 mg, 0.16 mmol), 2-aminopyrimidine-5-boronic acid (36 mg, 0.26 mmol), K₂CO₃ (72 mg, 0.52 mmol) in a mixture of DME/H₂O (4:1, 5 mL) was degassed with N₂ for 15 min. Pd(dppf)Cl₂ (25 mg, 0.03 mmol) was then added and the mixture was further degassed for another 15 min. The reaction vessel was then evacuated and backfilled with N₂ three times. The reaction mixture was heated to reflux under N₂ o.n. To this reaction mixture was added another portion of the boronic acid and catalyst and the reaction mixture was further allowed to heat to reflux under N₂ for 6 hr. The reaction mixture was then diluted with water and extracted with DCM (×2) The organic extracts were combined, dried over MgSO₄ and concentrated in vacuo. The resulting residue was purified using flash column chromatography (0-3% MeOH in DCM) to give a brown solid. The solid was re-dissolved in 1 N HCl solution and extracted with DCM (×2). The aqueous layer was then basified and extracted with DCM (×2). The organic extracts were combined, washed with brine, dried over MgSO₄ and concentrated in vacuo to give the title compound as light yellow powder (71 mg, 66%). ¹H NMR (401 MHz, Chloroform-d) δ 9.29 (s, 2H), 7.63-7.55 (m, 2H), 7.52-7.37 (m, 5H), 7.20 (br s, 1H), 5.28 (s, 2H), 4.84 (br s, 1H), 4.06 (app. t, 4H), 3.90 (app. t, 6H), 3.72 (br s, 2H), 2.75-2.64 (m, 1H), 2.63-2.50 (m, 1H), 2.49-2.29 (m, 4H), 2.19 (s, 3H), 1.41 (br s, 1H), 1.16 (d, J=6.5 Hz, 3H). LCMS: (Method B) Rf=2.363 min, (ESI) m/z: 635.3 ([M+H]⁺, 40%), 318.2 ([M+2H]²+, 100%).

### (S)-1-(6-(4-((((2-(2-Aminopyrimidin-5-yl)-7-methyl-4-morpholinothieno[3,2-d]pyrim-idin-6-yl)methyl)(methyl)amino)methyl)phenyl)-2-methyl-3,4-dihydroquinolin-1(2H)-yl)ethan-1-one [Cpd 043]

A solution of (S)-1-(6-(4-((((2-chloro-7-methyl-4-morpholinothieno[3,2-d]pyrimidin-6-yl)methyl)(methyl)amino)methyl)phenyl)-2-methyl-3,4-dihydroquinolin-1(2H)-yl)ethan-1-one (S19.2) (100 mg, 0.16 mmol), 2-aminopyrimidine-5-boronic acid (36 mg, 0.26 mmol), K₂CO₃ (72 mg, 0.52 mmol) in a mixture of DME/H₂O (4:1, 5 mL) was degassed with N₂ for 15 min. Pd(dppf)Cl₂ (25 mg, 0.03 mmol) was then added and the mixture was further degassed for another 15 min. The reaction vessel was then evacuated and backfilled with N₂ three times. The reaction mixture was heated to reflux under N₂ o.n. To this reaction mixture was added another portion of the boronic acid and catalyst and the reaction mixture was further allowed to heat to reflux under N₂ for 6 hr. The reaction mixture was then diluted with water and extracted with DCM (×2) The organic extracts were combined, dried over MgSO₄ and concentrated in vacuo. The resulting residue was purified using flash column chromatography (0-3% MeOH in DCM) to give a brown solid. The solid was re-dissolved in 1 N HCl solution and extracted with DCM (×2). The aqueous layer was then basified and extracted with DCM (×2). The organic extracts were combined, washed with brine, dried over MgSO₄ and concentrated in vacuo to give the title compound as an off-white powder (67 mg, 61%). ¹H NMR (401 MHz, Chloro form-d) δ 9.32 (s, 2H), 7.58 (d, J=8.2 Hz, 2H), 7.48 (d, J=7.9 Hz, 2H), 7.43 (dd, J=8.2, 2.2 Hz, 1H), 7.39 (sd, J=2.1 Hz, 1H), 7.19 (br s, 1H), 5.42 (s, 2H), 4.84 (br s, 1H), 4.11-3.98 (m, 4H), 3.95-3.81 (m, 6H), 3.74 (br s, 2H), 2.68 (dt, J=14.7, 4.8 Hz, 1H), 2.62-2.52 (m, 1H), 2.43 (s, 3H), 2.41-2.31 (m, 4H), 2.19 (s, 3H), 1.40 (br s, 1H), 1.15 (d, J=6.4 Hz, 3H). LCMS: (Method B) Rf=2.363 min, (ESI) m/z: 649.3 ([M+H]⁺, 30%), 325.2 ([M+2H]²+, 100%). HRMS (ESI⁺) calcd for [C₃₆H₄₀N₈O₂S+H]⁺649.3068, found 649.3080.

### (S)-N-(4-(1-Acetyl-2-methyl-1,2,3,4-tetrahydroquinolin-6-yl)phenethyl)-6-bromo-8-morpholinoimidazo[1,2-a]pyrazine-2-carboxamide (S17.i[Cpd 044])

To a solution of (S)-1-(6-(4-(2-aminoethyl)phenyl)-2-methyl-3,4-dihydroquinolin-1(2H)-yl)ethan-1-one (S9.17) (100 mg, 0.32 mmol) in DMF (5 mL) lithium(I) 6-bromo-8-morpholinoimidazo[1,2-a]pyrazine-2-carboxylate (S1.3) (130 mg, 0.39 mmol), EDC.HCl (124 mg, 0.65 mmol), HOBT (88 mg, 0.65 mmol) and DIPEA (0.34 mL, 1.94 mmol). The reaction mixture was stirred at r.t for 2 d. The reaction mixture was then poured into 5% Na₂CO₃ solution, the resulting precipitate was filtered and purified using flash column chromatography (0-3% MeOH in DCM) to give the title compound as a white foam which solidified under vacuum (105 mg, 53%). ¹H NMR (401 MHz, Chloroform-d) δ 7.98 (s, 1H), 7.62 (s, 1H), 7.54 (d with fine splitting, J=8.1 Hz, 2H), 7.42 (dd, J=8.0, 1.7 Hz, 1H), 7.38 (s, 1H), 7.33 (d with fine splitting, J=8.1 Hz, 2H), 7.25-7.11 (m, 2H), 4.85 (br s, 1H), 4.26 (br s, 4H), 3.81-3.71 (m, 6H), 2.99 (t, J=7.0 Hz, 2H), 2.69 (dt, J=14.5, 4.8 Hz, 1H), 2.64-2.53 (m, 1H), 2.46-2.31 (m, 1H), 2.20 (s, 3H), 1.41 (br s, 1H), 1.16 (d, J=6.4 Hz, 3H). LCMS: (Method C) Rf=4.008 min, (ESI) m/z: 617.2, 619.2 ([M+H]⁺, 100%).

### (S)-N-(4-(1-Acetyl-2-methyl-1,2,3,4-tetrahydroquinolin-6-yl)phenethyl)-2-chloro-4-morpholinothieno[3,2-d]pyrimidine-6-carboxamide (S17.i[Cpd 045])

To a solution of (S)-1-(6-(4-(2-aminoethyl)phenyl)-2-methyl-3,4-dihydroquinolin-1(2H)-yl)ethan-1-one (S9.17) (100 mg, 0.32 mmol) in DMF (5 mL) 2-chloro-4-morpholinothieno[3,2-d]pyrimidine-6-carboxylic acid (S15.10) (146 mg, 0.49 mmol), EDC.HCl (124 mg, 0.65 mmol), HOBT (88 mg, 0.65 mmol) and DIPEA (0.34 mL, 1.94 mmol). The reaction mixture was stirred at r.t for 1.5 d. The reaction mixture was then poured into 5% Na₂CO₃ solution, the resulting precipitate was filtered and purified using flash column chromatography (0-3% MeOH in DCM) to give an oil which was co-evaporated with DCM and diethyl ether to give the title compound as a white powder (115 mg, 63%). ¹H NMR (401 MHz, Chloroform-d) δ 7.61-7.47 (m, 3H), 7.40 (app. d, 2H), 7.34-7.27 (m, 2H), 7.19 (br s, 1H), 6.66 (br s, 1H), 4.84 (br s, 1H), 4.02 (s, 4H), 3.84 (s, 4H), 3.77 (br s, 2H), 3.00 (br s, 2H), 2.69 (dt, J=14.3, 4.5 Hz, 1H), 2.64-2.52 (m, 1H), 2.45-2.30 (m, 1H), 2.17 (s, 3H), 1.41 (br s, 1H), 1.16 (d, J=6.4 Hz, 3H). LCMS: (Method C) Rf=3.877 min, (ESI) m/z: 590.2 ([M+H]⁺, 100%).

### (S)-N-(4-(1-Acetyl-2-methyl-1,2,3,4-tetrahydroquinolin-6-yl)phenethyl)-6-(2-aminopy-rimidin-5-yl)-8-morpholinoimidazo[1,2-a]pyrazine-2-carboxamide [Cpd 044]

A solution of (S)-N-(4-(1-acetyl-2-methyl-1,2,3,4-tetrahydroquinolin-6-yl)phenethyl)-6-bromo-8-morpholinoimidazo[1,2-a]pyrazine-2-carboxamide (S17.i[Cpd 044]) (70 mg, 0.11 mmol), 2-aminopyrimidine-5-boronic acid (19 mg, 0.14 mmol), K₂CO₃ (47 mg, 0.34 mmol) in a mixture of DME/H₂O (4:1, 5 mL) was degassed with N₂ for 15 min. Pd(dppf)Cl₂ (8 mg, 0.01 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as a white powder (61 mg, 85%). ¹H NMR (400 MHz, DMSO-d₆) δ 8.77 (s, 2H), 8.44 (d, J=2.5 Hz, 2H), 8.23 (s, 1H), 7.61 (d, J=8.3 Hz, 2H), 7.53-7.44 (m, 2H), 7.41 (s, 1H), 7.34 (d, J=8.3 Hz, 2H), 6.89 (s, 2H), 4.64 (dd, J=12.7, 5.8 Hz, 1H), 4.35-4.20 (m, 4H), 3.83-3.71 (m, 4H), 3.62-3.49 (m, 2H), 2.91 (br t, J=7.6 Hz, 2H), 2.71 (dt, J=15.1, 5.4 Hz, 1H), 2.61-2.50 (m, 1H), 2.37-2.21 (m, 1H), 2.11 (s, 3H), 1.34 (br s, 1H), 1.05 (d, J=6.5 Hz, 3H). LCMS: (Method C) Rf=3.520 min, (ESI) m/z: 632.3 ([M+H]⁺, 100%). HRMS (ESI⁺) calcd for [C₃₅H₃₇N₉O₃+H]⁺632.3092, found 632.3106.

### (S)-N-(4-(1-acetyl-2-methyl-1,2,3,4-tetrahydroquinolin-6-yl)phenethyl)-2-(2-aminopy-rimidin-5-yl)-4-morpholinothieno[3,2-d]pyrimidine-6-carboxamide [Cpd 045]

A solution of (S)-N-(4-(1-acetyl-2-methyl-1,2,3,4-tetrahydroquinolin-6-yl)phenethyl)-2-chloro-4-morpholinothieno[3,2-d]pyrimidine-6-carboxamide (S17.i[Cpd 045]) (70 mg, 0.14 mmol), 2-aminopyrimidine-5-boronic acid (28 mg, 0.20 mmol), K₂CO₃ (56 mg, 0.41 mmol) in a mixture of DME/H₂O (4:1, 5 mL) was degassed with N₂ for 15 min. Pd(dppf)Cl₂ (20 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as an off-white powder (53 mg, 60%). ¹H NMR (401 MHz, DMSO-d₆) δ 9.11 (s, 2H), 9.05 (br t, J=5.6 Hz, 1H), 8.04 (s, 1H), 7.61 (d, J=8.3 Hz, 2H), 7.51-7.43 (m, 2H), 7.43-7.36 (m, 1H), 7.34 (d, J=8.2 Hz, 2H), 7.11 (s, 2H), 4.74-4.55 (m, 1H), 4.03-3.92 (m, 4H), 3.83-3.73 (m, 4H), 3.57 (app. q, 2H), 2.92 (br t, J=7.2 Hz, 2H), 2.70 (dt, J=15.1, 5.4 Hz, 1H), 2.59-2.51 (m, 1H), 2.34-2.21 (m, 1H), 2.10 (s, 3H), 1.33 (br s, 1H), 1.05 (d, J=6.4 Hz, 3H). LCMS: (Method C) Rf=3.540 min, (ESI) m/z: 649.3 ([M+H]⁺, 100%). HRMS (ESI⁺) calcd for [C₃₅H₃₆N₈O₃S+H]⁺649.2704, found 649.2716.

### (S)-N-(4-(1-Acetyl-2-methyl-1,2,3,4-tetrahydroquinolin-6-yl)phenethyl)-2-chloro-7-methyl-4-morpholinothieno[3,2-d]pyrimidine-6-carboxamide (S17.i[Cpd 046])

A solution of (S)-1-(6-(4-(2-aminoethyl)phenyl)-2-methyl-3,4-dihydroquinolin-1(2H)-yl)ethan-1-one (S9.17) (100 mg, 0.32 mmol), lithium(I) 2-chloro-7-methyl-4-morpholinothieno[3,2-d]pyrimidine-6-carboxylate (S16.5) (124 mg, 0.39 mmol), PyClock (360 mg, 0.65 mmol), DIPEA (0.34 mL, 1.94 mmol) in DMF (5 mL) was stirred at r.t for 24 hr. The reaction mixture was then poured into 5% Na₂CO₃ solution, the resulting precipitate was fil tered and purified using flash column chromatography (0-3% MeOH in DCM) to give an oil, which was co-evaporated with DCM and PE to give the title compound as a white powder (136 mg, 69%). ¹H NMR (401 MHz, Chloroform-d) δ 7.56 (d, J=8.1 Hz, 2H), 7.41 (dd, J=8.2, 1.8 Hz, 1H), 7.37 (s, 1H), 7.32 (d, J=8.1 Hz, 2H), 7.21 (br s, 1H), 6.09 (br t, J=4.8 Hz, 1H), 4.84 (br s, 1H), 4.07-3.92 (m, 4H), 3.89-3.74 (m, 6H), 3.02 (t, J=6.7 Hz, 2H), 2.69 (dt, J=14.5, 4.7 Hz, 1H), 2.58 (ddd, J=14.6, 11.1, 4.5 Hz, 1H), 2.43 (s, 3H), 2.41-2.32 (m, 1H), 2.19 (s, 3H), 1.41 (br s, 1H), 1.16 (d, J=6.4 Hz, 3H). LCMS: (Method C) Rf=4.021 min, (ESI) m/z: 604.2 ([M+H]⁺, 100%).

### (S)-N-(4-(1-Acetyl-2-methyl-1,2,3,4-tetrahydroquinolin-6-yl)phenethyl)-2-(2-aminopy-rimidin-5-yl)-7-methyl-4-morpholinothieno[3,2-d]pyrimidine-6-carboxamide [Cpd 046]

A solution of (S)-N-(4-(1-acetyl-2-methyl-1,2,3,4-tetrahydroquinolin-6-yl)phenethyl)-2-chloro-7-methyl-4-morpholinothieno[3,2-d]pyrimidine-6-carboxamide (S17.i[Cpd 046]) (100 mg, 0.50 mmol), 2-aminopyrimidine-5-boronic acid (35 mg, 0.25 mmol), K₂CO₃ (69 mg, 0.41 mmol) in a mixture of DME/H₂O (4:1, 5 mL) was degassed with N₂ for 15 min. Pd(dppf)Cl₂ (mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with cold MeOH to give the title compound as a white powder (70 mg, 65%). ¹H NMR (400 MHz, DMSO-d₆) δ 9.15 (s, 2H), 8.50 (br t, J=5.6 Hz, 1H), 7.61 (d, J=7.7 Hz, 2H), 7.54-7.44 (m, 2H), 7.40 (br s, 1H), 7.34 (d, J=7.8 Hz, 2H), 7.11 (s, 2H), 4.74-4.54 (m, 1H), 4.01-3.89 (m, 4H), 3.81-3.71 (m, 4H), 3.60-3.51 (m, 2H), 2.92 (br t, J=7.3 Hz, 2H), 2.71 (dt, J=15.6, 5.3 Hz, 1H), 2.61-2.51 (m, 1H), 2.34-2.20 (m, 1H), 2.10 (s, 3H), 1.33 (br s, 1H), 1.05 (d, J=6.4 Hz, 3H). LCMS: (Method B) Rf=2.874 min, (ESI) m/z: 663.3 ([M+H]⁺, 100%). HRMS (ESI⁺) calcd for [C₃₆H₃₈N₈O₃S+H]⁺663.2860, found 663.2874.

### (S)-N-(4-(1-Acetyl-2-methyl-1,2,3,4-tetrahydroquinolin-6-yl)phenyl)-2-aminoacetamide (S12.2)

Step 1: A solution of (S)-1-(6-(4-aminophenyl)-2-methyl-3,4-dihydroquinolin-1(2H)-yl)ethan-1-one (S9.14) (300 mg, 1.07 mmol), Boc-Gly-OH (281 mg, 1.60 mmol), HCTU (885 mg, 2.14 mmol), DIPEA (1.12 mL, 6.42 mmol) in DMF (5 mL) was stirred at r.t for 4 hr. The reaction mixture was diluted with EA and washed with 0.5 N HCl solution (×2), 5% NaOH solution (×2), dried over MgSO₄ and concentrated in vacuo to give S12.1. LCMS: (Method B) Rf=2.837 min, (ESI) m/z: 438.2 ([M+H]⁺, 10%), 364.2 ([M-(tert-butyl)+H]⁺, 100%). Step 2: The crude material was re-dissolved in a mixture of DCM and TFA (1:1, 8 mL) and the resulting reaction mixture was stirred at r.t for 1 hr. The reaction mixture was then basified to pH=14 with 5% NaOH solution and extracted with DCM (×2). The organic extracts were combined, dried over MgSO₄ and concentrated in vacuo. The resulting residue was triturated with DCM and PE to give the title compound as a light beige powder (316 mg, 87% over 2 steps). ¹H NMR (401 MHz, Chloroform-d) δ 9.50 (br s, 1H), 7.68 (d, J=8.2 Hz, 2H), 7.54 (d, J=8.2 Hz, 2H), 7.39 (d, J=8.2 Hz, 1H), 7.36 (s, 1H), 7.17 (br s, 1H), 4.83 (br s, 1H), 3.54 (s, 2H), 2.73-2.62 (m, 1H), 2.61-2.49 (m, 1H), 2.42-2.29 (m, 1H), 2.18 (s, 3H), 1.39 (br s, 1H), 1.15 (d, J=6.4 Hz, 3H). LCMS: (Method B) Rf=2.137 min, (ESI) m/z: 338.1 ([M+H]⁺, 100%).

### (S)-N-(3-(1-Acetyl-2-methyl-1,2,3,4-tetrahydroquinolin-6-yl)phenyl)-2-aminoacetamide (S12.4)

Step 1: A solution of (S)-1-(6-(3-aminophenyl)-2-methyl-3,4-dihydroquinolin-1(2H)-yl)ethan-1-one (S9.15) (300 mg, 1.07 mmol), Boc-Gly-OH (281 mg, 1.60 mmol), HCTU (885 mg, 2.14 mmol), DIPEA (1.12 mL, 6.42 mmol) in DMF (5 mL) was stirred at r.t for 4 hr. The reaction mixture was diluted with EA and washed with 0.5 N HCl solution (×2), 5% NaOH solution (×2), dried over MgSO₄ and concentrated in vacuo to give S12.3. LCMS: (Method B) Rf=2.830 min, (ESI) m/z: 338.2 ([M+H]⁺, 100%). Step 2: The crude material was re-dissolved in a mixture of DCM and TFA (1:1, 8 mL) and the resulting reaction mixture was stirred at r.t for 1 hr. The reaction mixture was then basified to pH=14 with 5% NaOH solution and extracted with DCM (×2). The organic extracts were combined, dried over MgSO₄ and concentrated in vacuo. The resulting residue then purified using flash column chromatography (0-5% MeOH in DCM+1% 7N NH₃ in MeOH) to give the title compound as an off-white semi solid (316 mg, 87% over 2 steps). ¹H NMR (401 MHz, Chloroform-d) δ 9.50 (s, 1H), 7.91 (s, 1H), 7.66-7.29 (m, 5H), 7.19 (br s, 1H), 4.84 (br s, 1H), 3.52 (s, 2H), 2.79-2.63 (m, 1H), 2.62-2.50 (m, 1H), 2.43-2.29 (m, 1H), 2.18 (s, 3H), 1.49-1.32 (m, 2H), 1.15 (br d, J=6.2 Hz, 5H). LCMS: (Method B) Rf=2.219 min, (ESI) m/z: 338.2 ([M+H]⁺, 100%).

### (S)-N-(2-((4-(1-Acetyl-2-methyl-1,2,3,4-tetrahydroquinolin-6-yl)phenyl)amino)-2-ox-oethyl)-2-chloro-7-methyl-4-morpholinothieno[3,2-d]pyrimidine-6-carboxamide (S17.I[Cpd 047])

A solution of (S)-N-(4-(1-acetyl-2-methyl-1,2,3,4-tetrahydroquinolin-6-yl)phenyl)-2-aminoacetamide (S12.2) (100 mg, 0.30 mmol), lithium(I) 2-chloro-7-methyl-4-morpholinothieno[3,2-d]pyrimidine-6-carboxylate (S16.5) (114 mg, 0.36 mmol), PyClock (329 mg, 0.59 mmol), DIPEA (0.31 mL, 1.78 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give the title compound as a white powder (110 mg, 59%). ¹H NMR (401 MHz, Chloroform-d) δ 8.65 (s, 1H), 7.65 (d, J=8.4 Hz, 2H), 7.55 (d, J=8.5 Hz, 2H), 7.39 (dd, J=8.2, 2.2 Hz, 1H), 7.36 (sd, J=2.0 Hz, 1H), 7.32 (d, J=4.5 Hz, 1H), 7.22 (br s, 1H), 4.83 (br s, 1H), 4.44 (d, J=4.6 Hz, 2H), 4.06-3.97 (m, 4H), 3.87-3.80 (m, 4H), 2.74 (s, 3H), 2.72-2.65 (m, 1H), 2.64-2.54 (m, 1H), 2.43-2.31 (m, 1H), 2.20 (s, 3H), 1.40 (br s, 1H), 1.16 (d, J=6.5 Hz, 3H). LCMS: (Method B) Rf=2.673 min, (ESI) m/z: 633.2 ([M+H]⁺, 100%).

### (S)-N-(2-((3-(1-Acetyl-2-methyl-1,2,3,4-tetrahydroquinolin-6-yl)phenyl)amino)-2-ox-oethyl)-2-chloro-7-methyl-4-morpholinothieno[3,2-d]pyrimidine-6-carboxamide (S17.i[Cpd 049])

A solution of (S)-N-(3-(1-acetyl-2-methyl-1,2,3,4-tetrahydroquinolin-6-yl)phenyl)-2-aminoacetamide (S12.4) (100 mg, 0.30 mmol), lithium(I) 2-chloro-7-methyl-4-morpholinothieno[3,2-d]pyrimidine-6-carboxylate (S16.5) (114 mg, 0.36 mmol), PyClock (329 mg, 0.59 mmol), DIPEA (0.31 mL, 1.78 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give the title compound as a white powder (130 mg, 69%). ¹H NMR (401 MHz, DMSO-d₆) δ 10.21 (s, 1H), 8.76 (t, J=5.8 Hz, 1H), 7.96 (s, 1H), 7.55 (dt, J=7.9, 1.6 Hz, 1H), 7.47-7.34 (m, 5H), 4.65 (tq, J=11.9, 6.4 Hz, 1H), 4.14 (d, J=5.8 Hz, 2H), 3.97-3.91 (m, 4H), 3.79-3.74 (m, 4H), 2.73 (dt, J=15.1, 5.3 Hz, 1H), 2.55 (s, 4H), 2.35-2.24 (m, 1H), 2.12 (s, 3H), 1.36 (br s, 1H), 1.06 (d, J=6.4 Hz, 3H). LCMS: (Method B) Rf=2.958 min, (ESI) m/z: 633.1 ([M+H]⁺, 100%).

### (S)-N-(2-((4-(1-Acetyl-2-methyl-1,2,3,4-tetrahydroquinolin-6-yl)phenyl)amino)-2-ox-oethyl)-6-bromo-3-methyl-8-morpholinoimidazo[1,2-a]pyrazine-2-carboxamide (S17.i[Cpd 048])

A solution of (S)-N-(4-(1-acetyl-2-methyl-1,2,3,4-tetrahydroquinolin-6-yl)phenyl)-2-aminoacetamide (S12.2) (100 mg, 0.30 mmol), lithium(I) 6-bromo-3-methyl-8-morpholinoimidazo[1,2-a]pyrazine-2-carboxylate (S14.3) (124 mg, 0.36 mmol), PyClock (329 mg, 0.59 mmol), DIPEA (0.31 mL, 1.78 mmol) in DMF (5 mL) was stirred at r.t o.n. The reaction mixture was then poured into 5% Na₂CO₃ solution, the resulting precipitate was filtered and purified using flash column chromatography (0-3% MeOH in DCM) to give the title compound as a white powder (138 mg, 69%). ¹H NMR (401 MHz, Chloroform-d) δ 8.67 (s, 1H), 7.94 (t, J=6.1 Hz, 1H), 7.61 (d, J=8.7 Hz, 2H), 7.52 (d, J=8.7 Hz, 2H), 7.41-7.37 (m, 2H), 7.35 (sd, J=2.0 Hz, 1H), 7.19 (br s, 1H), 4.83 (br s, 1H), 4.33 (br s, 4H), 4.30 (d, J=6.1 Hz, 2H), 3.89-3.82 (m, 4H), 2.74 (s, 3H), 2.71-2.64 (m, 1H), 2.63-2.50 (m, 1H), 2.43-2.30 (m, 1H), 2.19 (s, 3H), 1.40 (br s, 1H), 1.15 (d, J=6.4 Hz, 3H). LCMS: (Method B) Rf=2.991 min, (ESI) m/z: 660.2, 662.2 ([M+H]⁺, 100%).

### (S)-N-(2-((4-(1-Acetyl-2-methyl-1,2,3,4-tetrahydroquinolin-6-yl)phenyl)amino)-2-ox-oethyl)-2-(2-aminopyrimidin-5-yl)-7-methyl-4-morpholinothieno[3,2-d]pyrimidine-6-carboxamide [Cpd 047]

A solution of (S)-N-(2-((4-(1-acetyl-2-methyl-1,2,3,4-tetrahydroquinolin-6-yl)phenyl)amino)-2-oxoethyl)-2-chloro-7-methyl-4-morpholinothieno[3,2-d]pyrimidine-6-carboxamide (S17.I[Cpd 047]) (80 mg, 0.13 mmol), 2-aminopyrimidine-5-boronic acid (27 mg, 0.19 mmol), K₂CO₃ (53 mg, 0.38 mmol) in a mixture of DME/H₂O (4:1, 5 mL) was degassed with N₂ for 15 min. Pd(dppf)Cl₂ (19 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with a mixture of DCM and isopropanol (3:1, ×3). The organic extracts were combined, dried over MgSO₄ and concentrated in vacuo. The resulting residue was purified using flash column chromatography (0-6% MeOH in DCM) to give a brown solid, which was triturated with MeOH to give the title compound as a light beige powder (49 mg, 56%). ¹H NMR (401 MHz, DMSO-d₆) δ 10.22 (s, 1H), 9.19 (s, 2H), 8.70 (br t, J=5.8 Hz, 1H), 7.71 (d, J=8.9 Hz, 2H), 7.65 (d, J=8.8 Hz, 2H), 7.54-7.45 (m, 2H), 7.44-7.33 (m, 1H), 7.13 (s, 2H), 4.74-4.54 (m, 1H), 4.14 (d, J=5.8 Hz, 2H), 4.01 (app. t, 4H), 3.80 (app. t, 4H), 2.72 (dt, J=15.1, 5.3 Hz, 1H), 2.66 (s, 3H), 2.60-2.52 (m, 1H), 2.36-2.23 (m, 1H), 2.11 (s, 3H), 1.35 (br s, 1H), 1.06 (d, J=6.4 Hz, 3H). LCMS: (Method B) Rf=2.662 min, (ESI) m/z: 692.3 ([M+H]⁺, 100%). HRMS (ESI⁺) calcd for [C₃₆H₃₇N₉O₄S+H]⁺692.2762, found 692.2771.

### (S)-N-(2-((4-(1-Acetyl-2-methyl-1,2,3,4-tetrahydroquinolin-6-yl)phenyl)amino)-2-ox-oethyl)-6-(2-aminopyrimidin-5-yl)-3-methyl-8-morpholinoimidazo[1,2-a]pyrazine-2-carboxamide [Cpd 048]

A solution of (S)-N-(2-((4-(1-acetyl-2-methyl-1,2,3,4-tetrahydroquinolin-6-yl)phenyl)amino)-2-oxoethyl)-6-bromo-3-methyl-8-morpholinoimidazo[1,2-a]pyrazine-2-carboxamide (S17.i[Cpd 048]) (100 mg, 0.15 mmol), 2-aminopyrimidine-5-boronic acid (26 mg, 0.19 mmol), K₂CO₃ (63 mg, 0.46 mmol) in a mixture of DME/H₂O (4:1, 5 mL) was degassed with N₂ for 15 min. Pd(dppf)Cl₂ (11 mg, 0.02 mmol) was then added and the mixture was further degassed for another 15 min. The reaction vessel was then evacuated and backfilled with N₂ three times. The reaction mixture was heated to reflux under N₂ o.n. The resulting suspension was added water and the solid was filtered. The solid was re-suspended in a mixture of DCM and MeOH (1:1), and to this suspension was added TFA until fully dissolved. The solution was concentrated and the residue was purified using flash column chromatography (0-8% MeOH in DCM) to give a brown solid which was triturated with MeOH to give the title compound as a light beige powder (71 mg, 70%). II-1 NMR (401 MHz, DMSO-d₆) δ 10.17 (s, 1H), 8.91 (s, 2H), 8.59 (br t, J=6.0 Hz, 1H), 8.22 (s, 1H), 7.70 (d, J=8.8 Hz, 2H), 7.65 (d, J=8.8 Hz, 2H), 7.54-7.45 (m, 2H), 7.44-7.33 (m, 1H), 6.86 (s, 2H), 4.71-4.57 (m, 1H), 4.41-4.24 (m, 4H), 4.12 (d, J=5.9 Hz, 2H), 3.87-3.73 (m, 4H), 2.77 (s, 3H), 2.71 (dt, J=15.3, 5.7 Hz, 1H), 2.60-2.52 (m, 1H), 2.34-2.24 (m, 1H), 2.11 (s, 3H), 1.34 (br s, 1H), 1.06 (d, J=6.5 Hz, 3H). LCMS: (Method B) Rf=2.629 min, (ESI) m/z: 675.4 ([M+H]⁺, 100%). HRMS (ESI⁺) calcd for [C₃₆H₃₈N₁₀O₄+H]⁺675.3150, found 675.3157.

### (S)-N-(2-((3-(1-Acetyl-2-methyl-1,2,3,4-tetrahydroquinolin-6-yl)phenyl)amino)-2-ox-oethyl)-2-(2-aminopyrimidin-5-yl)-7-methyl-4-morpholinothieno[3,2-d]pyrimidine-6-carboxamide [Cpd 049]

A solution of (S)-N-(2-((3-(1-acetyl-2-methyl-1,2,3,4-tetrahydroquinolin-6-yl)phenyl)amino)-2-oxoethyl)-2-chloro-7-methyl-4-morpholinothieno[3,2-d]pyrimidine-6-carboxamide (S17.i[Cpd 049]) (100 mg, 0.16 mmol), 2-aminopyrimidine-5-boronic acid (34 mg, 0.24 mmol), K₂CO₃ (67 mg, 0.48 mmol) in a mixture of DME/H₂O (4:1, 5 mL) was degassed with N₂ for 15 min. Pd(dppf)Cl₂ (24 mg, 0.03 mmol) was then added and the mixture was further degassed for another 15 min. The reaction vessel was then evacuated and backfilled with N₂ three times. The reaction mixture was heated to reflux under N₂ o.n. The resulting suspension was added water and the solid was filtered. The solid was re-suspended in a mixture of DCM and MeOH (1:1), and to this suspension was added TFA until fully dissolved. The solution was concentrated and the residue was purified using flash column chromatography (0-8% MeOH in DCM) to a dark yellow solid which was triturated with MeOH to give the title compound as a white powder (69 mg, 63%). ¹H NMR (401 MHz, DMSO-d₆) δ 10.21 (s, 1H), 9.18 (s, 2H), 8.69 (br t, J=5.8 Hz, 1H), 7.97 (br st, J=1.9 Hz, 1H), 7.55 (dt, J=8.0, 1.6 Hz, 1H), 7.49-7.34 (m, 5H), 7.12 (s, 2H), 4.78-4.52 (m, 1H), 4.14 (d, J=5.8 Hz, 2H), 4.00 (app. t, 4H), 3.80 (app. t, 4H), 2.73 (dt, J=15.0, 5.4 Hz, 1H), 2.66 (s, 3H), 2.61-2.52 (m, 1H), 2.34-2.23 (m, 1H), 2.12 (s, 3H), 1.37 (br s, 1H), 1.06 (d, J=6.5 Hz, 3H). LCMS: (Method B) Rf=2.714 min, (ESI) m/z: 692.3 ([M+H]⁺, 100%). HRMS (ESI⁺) calcd for [C₃₆H₃₇N₉O₄S+H]⁺692.2762, found 6692.2776.

### tert-Butyl (3-bromophenethyl)carbamate (S9.18a)

To a solution of 3-bromophenethylamine (1 g, 5.00 mmol) in DCM (30 mL) was added di-tert-butyl dicarbonate (1.64 g, 7.51 mmol) and TEA (2.05 mL, 14.7 mmol). The reaction mixture was stirred at r.t o.n. The reaction mixture was diluted with DCM, washed with 0.5 N HCl solution (×2), sat. bicarb solution (×1), dried over MgSO₄ and concentrated in vacuo. The resulting residue was purified using flash column chromatography (0-10% EA in PE) to give the title compound as a clear oil (1.50 g, quant). ¹H NMR (401 MHz, CDCl₃) δ 7.40-7.32 (m, 2H), 7.20-7.09 (m, 2H), 4.53 (br s, 1H), 3.36 (q, J=6.8 Hz, 2H), 2.77 (t, J=7.0 Hz, 2H), 1.44 (s, 9H).

### tert-Butyl (3-(4,4,5,5-tetramethyl-1,3,2-dioxaborolan-2-yl)phenethyl)carbamate (S9.18b)

A solution of tert-butyl (3-bromophenethyl)carbamate (S9.18a) (1.5 g, 5.00 mmol), bis(pinacolato)diboron (2.55 g, 10.04 mmol) and KOAc (1.96 g, 19.97 mmol) in dry dioxane (15 mL) was degassed for 15 min. Pd(dppf)Cl₂ (24 mg, 0.03 mmol) was then added and the mixture was further degassed for another 15 min. The reaction vessel was then evacuated and backfilled with N₂ three times. The reaction mixture was heated at 80-90° C. for 18 hr. The reaction mixture was then filtered through celite and the filter pad rinsed with DCM. The filtrate was then concentrated in vacuo and the resulting residue was purified using flash column chromatography (0-10% EA in PE) to give the title compound as a clear oil which crystallized on standing (1.61 g, 93%). ¹H NMR (401 MHz, CDCl₃) δ 7.70-7.60 (m, 2H), 7.36-7.27 (m, 2H), 4.51 (br s, 1H), 3.38 (q, J=6.7 Hz, 2H), 2.80 (t, J=7.0 Hz, 2H), 1.43 (s, 9H), 1.35 (s, 12H).

### (S)-1-(6-(3-(2-Aminoethyl)phenyl)-2-methyl-3,4-dihydroquinolin-1(2H)-yl)ethan-1-one (S9.18)

Step 1: A solution of (S)-1-(6-bromo-2-methyl-3,4-dihydroquinolin-1(2H)-yl)ethan-1-one (S9.6) (300 mg, 1.12 mmol), tert-butyl (3-(4,4,5,5-tetramethyl-1,3,2-dioxaborolan-2-yl)phenethyl)carbamate (S9.18b) (466 mg, 1.34 mmol), K₂CO₃ (464 mg, 3.36 mmol) in a mixture of DME/H₂O (4:1, 5 mL) was degassed with N₂ for 15 min. N₂ three times. The reaction mixture was heated to reflux under N₂ for 4 hr. The mixture was then diluted with water and extracted with DCM (×2). The organic extracts were combined, washed with brine, dried over MgSO₄ and concentrated in vacuo to give an oily residue (Boc-S9.18). LCMS: (Method B) Rf=3.184 min, (ESI) m/z: 409.2 ([M+H]⁺, 100%). Step 2: The residue was re-dissolved in a mixture of DCM and TFA (1:1, 8 mL), the reaction mixture was stirred at r.t for 1 hr. The reaction mixture was then partitioned between 1 N HCl solution and DCM. The organic extract was discarded and the aqueous layer extracted again with DCM (×1). The aqueous layer was then basified to pH=14 with 10% NaOH solution and extracted with DCM (×3). The organic extracts were combined, dried over MgSO₄ and concentrated in vacuo. The residue was then purified using flash column chromatography chromatography (0-5% MeOH in DCM+1% 7N NH₃ in MeOH) to give the title compound as a pale yellow oil (309 mg, 90%). ¹H NMR (401 MHz, Chloroform-d) δ 7.51-7.31 (m, 5H), 7.24-7.07 (m, 2H), 4.83 (br s, 1H), 3.05 (t, J=6.9 Hz, 1H), 2.86 (t, J=6.9 Hz, 1H), 2.68 (dt, J=14.2, 4.8 Hz, 1H), 2.62-2.51 (m, 1H), 2.42-2.29 (m, 1H), 2.18 (s, 3H), 1.32 (br s, 1H), 1.15 (d, J=6.5 Hz, 3H). LCMS: (Method B) Rf=2.175 min, (ESI) m/z: 309.2 ([M+H]⁺, 100%).

### (S)-1-(2-Methyl-6-(1-(piperidin-4-yl)-1H-pyrazol-4-yl)-3,4-dihydroquinolin-1(2H)-yl)ethan-1-one (S9.19)

Step 1: A solution of (S)-1-(6-bromo-2-methyl-3,4-dihydroquinolin-1(2H)-yl)ethan-1-one (S9.6) (300 mg, 1.12 mmol), 1-(1-Boc-4-piperidyl)pyrazol-4-boronic acid pinacol ester (507 mg, 1.34 mmol), K₂CO₃ (464 mg, 3.36 mmol) in a mixture of DME/H₂O (4:1, 5 mL) was degassed with N₂ for 15 min. Pd(dppf)Cl₂ (82 mg, 0.11 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×2). The organic extracts were combined, washed with brine, dried over MgSO₄ and concentrated in vacuo. The residue was purified using flash column chromatography (0-100% EA) to give Boc-S9.19 as a white powder (426 mg, 87%). ¹H NMR (401 MHz, Chloroform-d) δ 7.76 (s, 1H), 7.65 (s, 1H), 7.30 (dd, J=8.1, 2.0 Hz, 1H), 7.27 (s, 1H), 7.11 (br s, 1H), 4.82 (br s, 1H), 4.38-4.16 (m, 3H), 2.91 (br t, J=12.6 Hz, 2H), 2.63 (dt, J=14.7, 4.6 Hz, 1H), 2.58-2.47 (m, 1H), 2.41-2.30 (m, 1H), 2.16 (m, 5H), 1.94 (qd, J=12.4, 4.4 Hz, 2H), 1.47 (s, 9H), 1.36 (br s, 1H), 1.13 (d, J=6.4 Hz, 3H). LCMS: (Method B) Rf=2.949 min, (ESI) m/z: 439.2 ([M+H]⁺, 20%), 383.3 ([M-(tert-butyl)+H]⁺, 100%). Step 2: The residue was re-dissolved in a mixture of DCM and TFA (1:1, 8 mL), the reaction mixture was stirred at r.t for 1 hr. To the reaction mixture was then added 10% NaOH (15 mL) and extracted with DCM (×3). The organic extracts were combined, dried over MgSO₄ and concentrated in vacuo to give the title compound as an off-white solid (304 mg, 92%). ¹H NMR (401 MHz, Chloroform-d) δ 7.76 (sd, J=0.6 Hz, 1H), 7.67 (sd, J=0.7 Hz, 1H), 7.31 (dd, J=8.1, 1.8 Hz, 1H), 7.27 (sd, J=1.9 Hz, 1H), 7.11 (br s, 1H), 4.82 (br s, 1H), 4.25 (tt, J=11.6, 4.0 Hz, 1H), 3.31-3.21 (m, 2H), 2.79 (td, J=12.4, 2.5 Hz, 2H), 2.63 (dt, J=14.7, 4.9 Hz, 1H), 2.58-2.46 (m, 1H), 2.35 (ddt, J=12.8, 7.5, 5.0 Hz, 1H), 2.25-2.17 (m, 2H), 2.15 (s, 3H), 2.02-1.86 (m, 4H), 1.36 (br s, 1H), 1.13 (d, J=6.5 Hz, 3H). LCMS: (Method B) Rf=2.084 min, (ESI) m/z: 339.2 ([M+H]⁺, 100%).

### (S)-1-(6-(4-((Hydroxyamino)methyl)phenyl)-2-methyl-3,4-dihydroquinolin-1(2H)-yl)ethan-1-one (S10.4)

Step 1: A solution of hydroxylamine hydrochloride (133 mg, 1.91 mmol) and sodium acetate (196 mg, 2.39 mmol) in 80% EtOH was stirred at r.t for 30 min. To this mixture was added (S)-4-(1-acetyl-2-methyl-1,2,3,4-tetrahydroquinolin-6-yl)benzaldehyde (S9.7) (350 mg, 1.19 mmol). The reaction mixture was then heated to reflux for 1 hr. The reaction mixture was then concentrated in vacuo. The residual solid was suspended in water and filtered to give the oxime (S10.3) as a white powder (330 mg). Step 2: The oxime was re-dissolved in MeOH (20 mL). To this mixture was added Conc. HCl (0.5 mL) and NaCNBH₃ (131 mg, 2.08 mmol) and stirred at r.t. After 3 hr, another portion of NaCNBH₃ was added and the reaction mixture was allowed to stir at r.t for another 3 hr. The reaction mixture was then quenched adding water (5 mL). and the MeOH removed in vacuo. The aqueous residue was acidified to pH=1 with 1 N HCl solution and extracted with DCM (×1). The aqueous layer was then basified to pH=12 with 5% NaOH solution and extracted with DCM (×2). The organic extracts were combined, dried over MgSO₄ and concentrated in vacuo to give the title compound as a white foam which solidified under vacuum (307 mg, 83% over two steps). ¹H NMR (401 MHz, Chloroform-d) δ 7.56 (d, J=8.2 Hz, 2H), 7.46 (d, J=8.2 Hz, 2H), 7.41-7.33 (m, 2H), 7.17 (br s, 1H), 4.82 (br s, 3H), 4.14 (s, 2H), 2.67 (dt, J=14.8, 5.0 Hz, 1H), 2.57 (ddd, J=14.8, 10.8, 4.9 Hz, 1H), 2.42-2.30 (m, 1H), 2.17 (s, 3H), 1.40 (br t, 1H), 1.15 (d, J=6.4 Hz, 3H). LCMS: (Method C) Rf=3.021 min, (ESI) m/z: 311.2 ([M+H]⁺, 100%).

### (S)-N-(3-(1-Acetyl-2-methyl-1,2,3,4-tetrahydroquinolin-6-yl)phenethyl)-6-bromo-3-methyl-8-morpholinoimidazo[1,2-a]pyrazine-2-carboxamide (S17.i[Cpd 050])

A solution of (S)-1-(6-(3-(2-aminoethyl)phenyl)-2-methyl-3,4-dihydroquinolin-1(2H)-yl)ethan-1-one (S9.18) (100 mg, 0.32 mmol), lithium(I) 6-bromo-3-methyl-8-morpholinoimidazo[1,2-a]pyrazine-2-carboxylate (S14.3) (140 mg, 0.40 mmol), PyClock (360 mg, 0.65 mmol), DIPEA (0.34 mL, 1.94 mmol) in DMF (5 mL) was stirred at r.t for 24 hr. The reaction mixture was then poured into 5% Na₂CO₃ solution, the resulting precipitate was filtered and purified using flash column chromatography (0-3% MeOH in DCM) to give an oil, which was co-evaporated with DCM and PE to give the title compound as a white powder (181 mg, 88%). ¹H NMR (401 MHz, Chloroform-d) δ 7.55-7.48 (m, 2H), 7.47-7.35 (m, 4H), 7.33-7.29 (m, 1H), 7.28-7.24 (m, 1H), 7.20 (br s, 1H), 4.85 (br s, 1H), 4.23 (app. t, 4H), 3.81-3.71 (m, 6H), 3.02 (t, J=6.9 Hz, 2H), 2.77 (s, 3H), 2.73-2.54 (m, 2H), 2.39 (dt, J=12.9, 6.3 Hz, 1H), 2.21 (s, 3H), 1.42 (br s, 1H), 1.18 (d, J=6.5 Hz, 3H). LCMS: (Method B) Rf=3.247 min, (ESI) m/z: 631.2, 633.2 ([M+H]⁺, 100%).

### (S)-N-(3-(1-acetyl-2-methyl-1,2,3,4-tetrahydroquinolin-6-yl)phenethyl)-6-(2-aminopy-rimidin-5-yl)-3-methyl-8-morpholinoimidazo[1,2-a]pyrazine-2-carboxamide [Cpd 050]

A solution of (S)-N-(3-(1-acetyl-2-methyl-1,2,3,4-tetrahydroquinolin-6-yl)phenethyl)-6-bromo-3-methyl-8-morpholinoimidazo[1,2-a]pyrazine-2-carboxamide (S17.i[Cpd 050]) (90 mg, 0.14 mmol), 2-aminopyrimidine-5-boronic acid (24 mg, 0.17 mmol), K₂CO₃ (138 mg, 0.42 mmol) in a mixture of DME/H₂O (4:1, 5 mL) was degassed with N₂ for 15 min. Pd(dppf)Cl₂ (11 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with a small amount of cold MeOH to give the title compound as a light beige powder (65 mg, 71%). ¹H NMR (401 MHz, DMSO-d₆) δ 8.89 (s, 2H), 8.27 (br t, J=6.0 Hz, 1H), 8.18 (s, 1H), 7.56-7.33 (m, 6H), 7.25 (d, J=7.5 Hz, 1H), 6.86 (s, 2H), 4.69-4.54 (m, 1H), 4.33-4.12 (m, 4H), 3.78-3.67 (m, 4H), 3.58 (app. q, 2H), 2.94 (br t, J=7.3 Hz, 2H), 2.74 (s, 3H), 2.62 (dt, J=15.0, 5.6 Hz, 1H), 2.49-2.40 (m, 1H), 2.31-2.18 (m, 1H), 2.09 (s, 3H), 1.33 (br s, 1H), 1.04 (d, J=6.4 Hz, 3H). LCMS: (Method B) Rf=2.832 min, (ESI) m/z: 646.3 ([M+H]⁺, 100%). HRMS (ESI⁺) calcd for [C₃₆H₃₉N₉O₃+H]⁺646.3249, found 646.3261.

### (S)-N-(3-(1-Acetyl-2-methyl-1,2,3,4-tetrahydroquinolin-6-yl)phenethyl)-2-chloro-7-methyl-4-morpholinothieno[3,2-d]pyrimidine-6-carboxamide (S17.i[Cpd 051])

A solution of (S)-1-(6-(4-(2-aminoethyl)phenyl)-2-methyl-3,4-dihydroquinolin-1(2H)-yl)ethan-1-one (S9.17) (100 mg, 0.32 mmol), lithium(I) 2-chloro-7-methyl-4-morpholinothieno[3,2-d]pyrimidine-6-carboxylate (S16.5) (124 mg, 0.39 mmol), PyClock (360 mg, 0.65 mmol), DIPEA (0.34 mL, 1.94 mmol) in DMF (5 mL) was stirred at r.t for 24 hr. The reaction mixture was then poured into 5% Na₂CO₃ solution, the resulting precipitate was fil tered and purified using flash column chromatography (0-3% MeOH in DCM) to give an oil, which was co-evaporated with DCM and PE to give the title compound as an off-white powder (180 mg, 91%). ¹H NMR (401 MHz, Chloroform-d) δ 7.53-7.44 (m, 2H), 7.45-7.36 (m, 2H), 7.35 (d, J=2.0 Hz, 1H), 7.27-7.12 (m, 2H), 6.10 (br t, J=4.9 Hz, 1H), 4.83 (br s, 1H), 4.06-3.93 (m, 4H), 3.88-3.73 (m, 6H), 3.05 (t, J=6.8 Hz, 2H), 2.66 (dt, J=14.9, 5.1 Hz, 1H), 2.57 (dp, J=14.7, 5.0 Hz, 1H), 2.42 (s, 3H), 2.36 (ddt, J=10.2, 7.6, 3.7 Hz, 1H), 2.19 (s, 3H), 1.40 (br s, 1H), 1.16 (d, J=6.5 Hz, 3H). LCMS: (Method B) Rf=3.145 min, (ESI) m/z: 604.2 ([M+H]⁺, 100%).

### (S)-N-(3-(1-Acetyl-2-methyl-1,2,3,4-tetrahydroquinolin-6-yl)phenethyl)-2-(2-aminopy-rimidin-5-yl)-7-methyl-4-morpholinothieno[3,2-d]pyrimidine-6-carboxamide [Cpd 051]

A solution of (S)-N-(3-(1-acetyl-2-methyl-1,2,3,4-tetrahydroquinolin-6-yl)phenethyl)-2-chloro-7-methyl-4-morpholinothieno[3,2-d]pyrimidine-6-carboxamide (S17.i[Cpd 051]) (90 mg, 0.14 mmol), 2-aminopyrimidine-5-boronic acid (32 mg, 0.23 mmol), K₂CO₃ (62 mg, 0.45 mmol) in a mixture of DME/H₂O (4:1, 5 mL) was degassed with N₂ for 15 min. Pd(dppf)Cl₂ (22 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with a small amount of cold MeOH to give the title compound as an off-white powder (67 mg, 68%). ¹H NMR (401 MHz, DMSO-d₆) δ 9.15 (s, 2H), 8.49 (br t, J=5.5 Hz, 1H), 7.58-7.35 (m, 6H), 7.25 (d, J=7.5 Hz, 1H), 7.11 (s, 2H), 4.73-4.53 (m, 1H), 3.95 (app. t, 4H), 3.76 (app. t, 4H), 3.59 (dt, J=6.7 Hz, 2H), 2.96 (br t, J=7.0 Hz, 2H), 2.65 (dt, J=15.1, 5.4 Hz, 1H), 2.56-2.51 (m, 1H), 2.45 (s, 3H), 2.31-2.19 (m, 1H), 2.10 (s, 3H), 1.32 (br s, 1H), 1.04 (d, J=6.4 Hz, 3H). LCMS: (Method B) Rf=2.885 min, (ESI) m/z: 663.3 ([M+H]⁺, 100%). HRMS (ESI⁺) calcd for [C₃₆H₃₈N₈O₃S+H]⁺663.2860, found 663.2871.

### (S)-1-(6-(1-(1-((2-Chloro-7-methyl-4-morpholinothieno[3,2-d]pyrimidin-6-yl)methyl)pi-peridin-4-yl)-1H-pyrazol-4-yl)-2-methyl-3,4-dihydroquinolin-1(2H)-yl)ethan-1-one (S17.i[Cpd 052])

To a solution of (S)-1-(2-methyl-6-(1-(piperidin-4-yl)-1H-pyrazol-4-yl)-3,4-dihydro-quinolin-1(2H)-yl)ethan-1-one (S9.19) (100 mg, 0.30 mmol), 2-chloro-7-methyl-4-morpholinothieno[3,2-d]pyrimidine-6-carbaldehyde (S16.7) (264 mg, 0.89 mmol) in dry DCE was added glacial acetic acid (0.34 mL, 5.9 mmol) and sodium triacetoxyborohydride (250 mg, 1.18 mmol). The reaction vessel was then evacuated and backfilled with N₂ three times. The reaction mixture was heated to reflux under N₂ o.n. The reaction mixture was then filtered through celite and the filter pad rinsed with DCM. The filtrated was washed with 2% NaOH solution, dried over MgSO₄ and the solvent concentrated in vacuo. The residue was then purified using flash column chromatography (0-3% MeOH in DCM) to give an oil which co-evaporated with DCM and PE to give the title compound as an off-white powder (143 mg, 78%). ¹H NMR (401 MHz, Chloroform-d) δ 7.80 (s, 1H), 7.71 (s, 1H), 7.33 (dd, J=8.1, 2.1 Hz, 1H), 7.30 (s, 1H), 7.14 (br s, 1H), 4.85 (br s, 1H), 4.29-4.14 (m, 1H), 4.08-3.96 (m, 4H), 3.94-3.77 (m, 6H), 3.15 (br d, J=11.0 Hz, 2H), 2.65 (dt, J=14.6, 4.5 Hz, 1H), 2.56 (ddd, J=17.2, 12.1, 3.2 Hz, 1H), 2.45-2.29 (m, 6H), 2.29-2.10 (m, 7H), 1.38 (br s, 1H), 1.16 (d, J=6.4 Hz, 3H). LCMS: (Method B) Rf=2.476 min, (ESI) m/z: 620.2 ([M+H]⁺, 100%).

### (S)-1-(6-(1-(1-((2-(2-Aminopyrimidin-5-yl)-7-methyl-4-morpholinothieno[3,2-d]pyrim-idin-6-yl)methyl)piperidin-4-yl)-1H-pyrazol-4-yl)-2-methyl-3,4-dihydroquinolin-1(2H)-yl)ethan-1-one [Cpd 052]

A solution of (S)-1-(6-(1-(1-((2-Chloro-7-methyl-4-morpholinothieno[3,2-d]pyrim-idin-6-yl)methyl)piperidin-4-yl)-1H-pyrazol-4-yl)-2-methyl-3,4-dihydroquinolin-1(2H)-yl)ethan-1-one (S17.i[Cpd 052]) (100 mg, 0.30 mmol), 2-aminopyrimidine-5-boronic acid (46 mg, 0.33 mmol), K₂CO₃ (67 mg, 0.48 mmol) in a mixture of DME/H₂O (4:1, 5 mL) was degassed with N₂ for 15 min. Pd(dppf)Cl₂ (120 mg, 0.16 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-6% MeOH in DCM) to give a brown solid, which was triturated with a small amount of cold MeOH to give the title compound as an off-white powder (71 mg, 65%). ¹H NMR (401 MHz, DMSO-d₆) δ 9.15 (s, 2H), 8.24 (s, 1H), 7.86 (s, 1H), 7.48-7.36 (m, 2H), 7.29 (s, 1H), 7.07 (s, 2H), 4.63 (br s, 1H), 4.27-4.13 (m, 1H), 3.94 (app. t, 4H), 3.86 (s, 2H), 3.78 (app. t, 4H), 3.02 (br d, J=11.0 Hz, 2H), 2.62 (dq, J=10.0, 5.0 Hz, 1H), 2.49-2.40 (m, 1H), 2.36 (s, 3H), 2.33-2.21 (m, 3H), 2.12-1.93 (m, 7H), 1.29 (br s, 1H), 1.03 (d, J=6.4 Hz, 3H). LCMS: (Method B) Rf=2.324 min, (ESI) m/z: 679.3 ([M+H]⁺, 40%), 340.2 ([M+2H]²+, 100%). HRMS (ESI⁺) calcd for [C₃₆H₄₂N₁₀O₂S+H]⁺679.3286, found 679.3298.

### (S)-1-(6-(1-(1-(2-Chloro-7-methyl-4-morpholinothieno[3,2-d]pyrimidine-6-carbonyl)pi-peridin-4-yl)-1H-pyrazol-4-yl)-2-methyl-3,4-dihydroquinolin-1(2H)-yl)ethan-1-one (S17.i[Cpd 053])

A solution of (S)-1-(2-methyl-6-(1-(piperidin-4-yl)-1H-pyrazol-4-yl)-3,4-dihydroquin-olin-1(2H)-yl)ethan-1-one (59.19) (100 mg, 0.32 mmol), lithium(I) 2-chloro-7-methyl-4-morpholinothieno[3,2-d]pyrimidine-6-carboxylate (516.5) (113 mg, 0.35 mmol), PyClock (328 mg, 0.59 mmol), DIPEA (0.31 mL, 1.77 mmol) in DMF (5 mL) was stirred at r.t for 24 hr. The reaction mixture was then poured into 5% Na₂CO₃ solution, the resulting precipitate was fil tered and purified using flash column chromatography (0-3% MeOH in DCM) to give the title compound as an off-white powder (104 mg, 56%). ¹H NMR (401 MHz, Chloroform-d) δ 7.81 (sd, J=0.7 Hz, 1H), 7.69 (sd, J=0.8 Hz, 1H), 7.33 (dd, J=8.2, 2.0 Hz, 1H), 7.29 (sd, J=1.4 Hz, 1H), 7.16 (br s, 1H), 4.98-3.95 (br s, 2H), 4.84 (br s, 1H), 4.46 (tt, J=11.0, 4.0 Hz, 1H), 4.08-3.95 (m, 4H), 3.90-3.78 (m, 4H), 3.24 (s, 2H), 2.66 (dt, J=14.9, 5.0 Hz, 1H), 2.56 (tt, J=11.4, 5.2 Hz, 1H), 2.45 (s, 3H), 2.38 (ddt, J=9.9, 7.2, 5.0 Hz, 1H), 2.30 (d, J=10.5 Hz, 2H), 2.18 (s, 3H), 2.16-2.04 (m, 2H), 1.36 (br s, 1H), 1.16 (d, J=6.5 Hz, 3H). LCMS: (Method B) Rf=2.892 min, (ESI) m/z: 634.3 ([M+H]⁺, 100%).

### (S)-1-(6-(1-(1-(2-(2-Aminopyrimidin-5-yl)-7-methyl-4-morpholinothieno[3,2-d]pyrimi-dine-6-carbonyl)piperidin-4-yl)-1H-pyrazol-4-yl)-2-methyl-3,4-dihydroquinolin-1(2H)-yl)ethan-1-one [Cpd 053]

A solution of (S)-1-(6-(1-(1-(2-chloro-7-methyl-4-morpholinothieno[3,2-d]pyrimidine-6-carbonyl)piperidin-4-yl)-1H-pyrazol-4-yl)-2-methyl-3,4-dihydroquinolin-1(2H)-yl)ethan-1-one (S17.i[Cpd 053]) (70 mg, 0.11 mmol), 2-aminopyrimidine-5-boronic acid (23 mg, 0.17 mmol), K₂CO₃ (46 mg, 0.33 mmol) in a mixture of DME/H₂O (4:1, 5 mL) was degassed with N₂ for 15 min. Pd(dppf)Cl₂ (23 mg, 0.03 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-4% MeOH in DCM+1% 7N NH₃ in MeOH) to give a brown solid, which was triturated with a small amount of cold MeOH to give the title compound as a light yellow powder (47 mg, 61). ¹H NMR (401 MHz, DMSO-d₆) δ 9.17 (s, 2H), 8.27 (sd, J=0.8 Hz, 1H), 7.88 (sd, J=0.7 Hz, 1H), 7.47-7.37 (m, 2H), 7.30 (s, 1H), 7.12 (s, 2H), 4.79-4.35 (m, 3H), 4.02-3.90 (m, 4H), 3.84-3.73 (m, 4H), 3.21 (br s, 2H), 2.63 (dt, J=15.0, 5.4 Hz, 1H), 2.49-2.43 (m, 1H), 2.39 (s, 3H), 2.35-2.22 (m, 1H), 2.20-2.02 (m, 5H), 1.92 (br s, 2H), 1.29 (br s, 1H), 1.03 (d, J=6.5 Hz, 3H). LCMS: (Method B) Rf=2.610 min, (ESI) m/z: 693.3 ([M+H]⁺, 100%). HRMS (ESI⁺) calcd for [C₃₆H₄₀N₁₀O₃S+H]⁺693.3078, found 693.3089.

### tert-Butyl (S)-(4-(1-acetyl-2-methyl-1,2,3,4-tetrahydroquinolin-6-yl)benzyl)((2-chloro-7-methyl-4-morpholinothieno[3,2-d]pyrimidin-6-yl)methyl)carbamate (S21.3)

Step 1: To a sealed tube was added (S)-1-(6-(4-(aminomethyl)phenyl)-2-methyl-3,4-dihydroquinolin-1(2H)-yl)ethan-1-one (59.12) (100 mg, 0.34 mmol), 2-chloro-7-methyl-4-morpholinothieno[3,2-d]pyrimidine-6-carbaldehyde (S16.7) (112 mg, 0.38 mmol) and MeOH (4 mL). The resulting suspension was heated at 80° C. for 3 hr. The resulting precipitate was filter and washed with MeOH to give the imine (S21.1) as an off-white powder (175 mg). Step 2: The imine intermediate was re-dissolved in a mixture of MeOH and THE (4:1). To this mixture was added NaBH₄ (117 mg, 3.09 mmol). The reaction mixture was stirred at r.t. After 3 hr, the reaction was quenched by 5% NaOH soln (10 mL). The organic solvent was removed in vacuo. The remaining aqueous residue was extracted with DCM (×2). The organic extracts were combined, dried over MgSO₄ and concentrated in vacuo to give the free amine (S21.2). LCMS: (Method B) Rf=2.442 min, (ESI) m/z: 576.2 ([M+H]⁺, 100%). Step 3: The amine intermediate was dissolved in DCM (10 mL) and to this mixture was added di-tert-butyl dicarbonate (100 mg, 0.46 mmol) and TEA (127 uL, 0.92 mmol). The reaction mixture was stirred at r.t o.n. The reaction mixture was then diluted with DCM, washed with 0.5 N HCl solution (×2), sat. bicarb solution (×1), dried over MgSO₄ and concentrated in vacuo. The resulting residue was then purified using flash column chromatography (0-50% EA in DCM) to give the title compound as a clear oil which crystallized upon scratching (190 mg, 83% over 3 steps). ¹H NMR (401 MHz, Chloroform-d) δ 7.54 (d, J=8.2 Hz, 2H), 7.41 (dd, J=8.1, 2.2 Hz, 1H), 7.37 (s, 1H), 7.33-7.26 (m, 2H), 7.22 (br s, 1H), 4.85 (br s, 1H), 4.72-4.39 (m, 4H), 4.02-3.90 (m, 4H), 3.88-3.73 (m, 4H), 2.70 (dt, J=14.8, 5.0 Hz, 1H), 2.64-2.53 (m, 1H), 2.44-2.33 (m, 1H), 2.25 (s, 3H), 2.20 (s, 3H), 1.53 (s, 9H), 1.45-1.34 (m, 1H), 1.16 (d, J=6.5 Hz, 3H). LCMS: (Method C) Rf=3.019 min, (ESI) m/z: 676.3 ([M+H]⁺, 100%).

### tert-Butyl (S)-(4-(1-acetyl-2-methyl-1,2,3,4-tetrahydroquinolin-6-yl)benzyl)((2-(2-amino-pyrimidin-5-yl)-7-methyl-4-morpholinothieno[3,2-d]pyrimidin-6-yl)methyl)carbamate [Cpd 054]

A solution of tert-butyl (S)-(4-(1-acetyl-2-methyl-1,2,3,4-tetrahydroquinolin-6-yl)benzyl)((2-chloro-7-methyl-4-morpholinothieno[3,2-d]pyrimidin-6-yl)methyl)carbamate (521.3) (160 mg, 0.24 mmol), 2-aminopyrimidine-5-boronic acid (50 mg, 0.36 mmol), K₂CO₃ (100 mg, 0.72 mmol) in a mixture of DME/H₂O (4:1, 5 mL) was degassed with N₂ for 15 min. Pd(dppf)Cl₂ (35 mg, 0.05 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×2). The organic extracts were combined, dried over MgSO₄ and concentrated in vacuo. The resulting residue was purified using flash column chromatography (0-4% MeOH in DCM) to give the title compound as an oil which was co-evaporated with DCM and PE to give the title compound as a white powder (165 mg, 95%). ¹H NMR (401 MHz, Chloroform-d) δ 9.41 (br s, 2H), 7.57 (d, J=6.7 Hz, 2H), 7.45-7.29 (m, 4H), 6.50 (br t, 2H), 4.84 (s, 1H), 4.67 (app. br d, 2H), 4.52 (app. br d, 2H), 4.02 (br s, 4H), 3.87 (br s, 4H), 2.80-2.65 (m, 1H), 2.64-2.51 (m, 1H), 2.42-2.29 (m, 4H), 2.20 (s, 3H), 1.55 (s, 9H), 1.42 (br s, 1H), 1.17 (d, J=6.0 Hz, 3H). LCMS: (Method B) Rf=3.339 min, (ESI) m/z: 735.3 ([M+H]⁺, 30%), 368.2 ([M+2H]²+, 100%). HRMS (ESI⁺) calcd for [C₄₀H₄₆N₈O₄S+H]⁺735.3435, found 735.3447.

### (S)-1-(6-(4-((((2-(2-Aminopyrimidin-5-yl)-7-methyl-4-morpholinothieno[3,2-d]pyrim-idin-6-yl)methyl)amino)methyl)phenyl)-2-methyl-3,4-dihydroquinolin-1(2H)-yl)ethan-1-one [Cpd 055]

A solution of tert-butyl (S)-(4-(1-acetyl-2-methyl-1,2,3,4-tetrahydroquinolin-6-yl)benzyl)((2-(2-aminopyrimidin-5-yl)-7-methyl-4-morpholinothieno[3,2-d]pyrimidin-6-yl)methyl)carbamate [Cpd 054] (160 mg 0.22 mmol) in a mixture of DCM and TFA (8 mL) was stirred at r.t for 1 hr. The reaction mixture was then partitioned between 1 N HCl solution and DCM. The organic extract was discarded and the aqueous layer extracted again with DCM (×1). The aqueous layer was then basified to pH=14 with 10% NaOH solution and extracted with DCM (×3). The organic extracts were combined, dried over MgSO₄ and concentrated in vacuo to give the title compound as an off-white powder (113 mg, 82%). ¹H NMR (401 MHz, DMSO-d₆) δ 9.15 (s, 2H), 7.63 (d, J=8.3 Hz, 2H), 7.53-7.47 (m, 2H), 7.46-7.35 (m, 3H), 7.08 (s, 2H), 4.73-4.55 (m, 1H), 3.98 (s, 2H), 3.96-3.91 (m, 4H), 3.81 (s, 2H), 3.79-3.74 (m, 4H), 3.15 (br s, 1H), 2.71 (dt, J=15.1, 5.3 Hz, 1H), 2.60-2.51 (m, 1H), 2.34-2.22 (m, 4H), 2.11 (s, 3H), 1.34 (br s, 1H), 1.06 (d, J=6.4 Hz, 3H). LCMS: (Method B) Rf=2.338 min, (ESI) m/z: 635.3 ([M+H]⁺, 100%). HRMS (ESI⁺) calcd for [C₃₅H₃₈N₈O₂S+H]⁺635.2911, found 635.2923.

### (S)-N-(4-(1-Acetyl-2-methyl-1,2,3,4-tetrahydroquinolin-6-yl)benzyl)-N-((2-(2-aminopy-rimidin-5-yl)-7-methyl-4-morpholinothieno[3,2-d]pyrimidin-6-yl)methyl)acetamide [Cpd 056]

To a solution of (S)-1-(6-(4-((((2-(2-aminopyrimidin-5-yl)-7-methyl-4-morpholinothieno[3,2-d]pyrimidin-6-yl)methyl)amino)methyl)phenyl)-2-methyl-3,4-dihydroquinolin-1(2H)-yl)ethan-1-one [Cpd 055] (15 mg, 0.02 mmol) in dry DCM at 0° C. was added TEA (14 uL, 0.10 mmol) and acetyl chloride (6 uL, 0.05 mmol). The reaction mixture was stirred at this temperature for 15 min. The reaction was then quenched with 2% NaOH solution and extracted with DCM (×2). The organic extracts were combined, dried over MgSO₄ and concentrated in vacuo. The resulting residue was purified using flash column chromatography (0-3% MeOH in DCM) to give the title compound as a white powder (10 mg, 63%). LCMS: (Method B) Rf=2.723 min, (ESI) m/z: 677.3 ([M+H]⁺, 100%). HRMS (ESI⁺) calcd for [C₃₇H₄₀N₈O₃S+H]⁺677.3017, found 677.3030.

### (S)-N-(4-(1-Acetyl-2-methyl-1,2,3,4-tetrahydroquinolin-6-yl)benzyl)-6-(6-amino-4-(tri-fluoromethyl)pyridin-3-yl)-8-morpholinoimidazo[1,2-a]pyrazine-2-carboxamide [Cpd 057]

A solution of (S)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010]) (90 mg, 0.15 mmol), 2-Amino-4-(trifluoromethyl)pyridine-5-boronic acid pinacol ester (52 mg, 0.18 mmol), K₂CO₃ (62 mg, 0.45 mmol) in a mixture of DME/H₂O (4:1, 5 mL) was degassed with N₂ for 15 min. Pd(dppf)Cl₂ (11 mg, 0.02 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, dried over MgSO₄ and concentrated in vacuo. The resulting residue was purified using flash column chromatography (0-4% MeOH in DCM+1% 7N NH₃ in MeOH) to the title compound as an off-white powder (44 mg, 43%). ¹H NMR (401 MHz, Chloroform-d) δ 8.23 (s, major rotamer), 8.14 (s, major rotamer), 7.66-7.51 (m), 7.49-7.36 (m), 7.19 (br s, 1H), 6.88 (s, major rotamer), 5.13, 4.69 (two br s, 2H, pair of rotamers), 4.83 (br s, 1H), 4.75 (d, J=6.2 Hz, 2H), 4.28 (app. t, 4H), 3.83 (app. t, 4H), 2.74-2.64 (m, 1H), 2.63-2.52 (m, 1H), 2.42-2.31 (m, 1H), 2.19 (s, 3H), 1.40 (br s, 1H), 1.16 (d, J=6.4 Hz, 3H). Minor rotamer peaks were significantly overlapped with the two multiplets (7.66-7.51), (7.49-7.36) and could not be distinguished. LCMS: (Method B) Rf=2.809 min, (ESI) m/z: 685.3 ([M+H]⁺, 100%). HRMS (ESI⁺) calcd for [C₃₆H₃₅F₃N₈O₃+H]⁺685.2857, found 685.2870.

### (S)-N-(4-(1-Acetyl-2-methyl-1,2,3,4-tetra-hydroquinolin-6-yl)benzyl)-6-(6-methoxypyridin-3-yl)-8-morpholinoimidazo[1,2-a]pyrazine-2-carboxamide [Cpd 058]

A mixture of (S)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010]) (90 mg, 0.15 mmol), 6-methoxy-3-pyridinylboronic acid (31 mg, 0.20 mmol), K₂CO₃ (69 mg, 0.50 mmol) in a mixture of DME/H₂O (4:1, 5 mL) was degassed with N₂ for 15 min. Pd(dppf)Cl₂ (11 mg, 0.02 mmol) was then added and the mixture was further degassed for another 15 min. The reaction vessel was then evacuated and backfilled with N₂ three times. The reaction mixture was heated to reflux under N₂ o.n. The mixture was then diluted with 2% NaOH solution and extracted with CHCl₃/isopropanol (3:1, ×2). The organic extracts were combined, dried over MgSO₄ and concentrated in vacuo. The resulting residue was purified using flash column chromatography (0-5% MeOH in DCM) to give a yellow solid, which was triturated with cold MeOH to give the title compound as a white powder (72 mg, 77%). ¹H NMR (401 MHz, DMSO-d₆) δ 9.03 (br t, J=6.4 Hz, 1H), 8.78 (dd, J=2.5, 0.7 Hz, 1H), 8.55 (s, 1H), 8.33 (s, 1H), 8.23 (dd, J=8.7, 2.5 Hz, 1H), 7.64 (d, J=8.3 Hz, 2H), 7.53-7.45 (m, 2H), 7.41 (d, J=8.1 Hz, 3H), 6.93 (dd, J=8.7, 0.7 Hz, 1H), 4.70-4.60 (m, 1H), 4.55 (d, J=6.3 Hz, 2H), 4.32 (app. t, 4H), 3.90 (s, 3H), 3.80 (app. t, 4H), 2.71 (dt, J=15.2, 5.4 Hz, 1H), 2.59-2.52 (m, 1H), 2.34-2.23 (m, 1H), 2.11 (s, 3H), 1.34 (br s, 1H), 1.06 (d, J=6.5 Hz, 3H). LCMS: (Method B) Rf=2.941 min, (ESI) m/z: 632.3 ([M+H]⁺, 100%). HRMS (ESI⁺) calcd for [C₃₆H₃₇N₇O₄+H]⁺632.2980, found 632.2992.

### (S)-N-(4-(1-Acetyl-2-methyl-1,2,3,4-tetrahydroquinolin-6-yl)benzyl)-6-(1H-indazol-4-yl)-8-morpholinoimidazo[1,2-a]pyrazine-2-carboxamide [Cpd 059]

To a mixture of (S)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010]) (90 mg, 0.15 mmol), indazole-4-boronic acid (33 mg, 0.17 mmol), Na₂CO₃ (53 mg, 0.50 mmol) in degassed toluene/EtOH/water (6:3:1, 3 mL) was added PdCl₂(PPh₃)₂ (6 mg, 0.01 mmol) and the mixture was further degassed with N₂ for another 5 min. The reaction mixture was heated at 120° C. under microwave irradiation for 1 hr. The mixture was then diluted with 2% NaOH solution and extracted with DCM (×2). The organic extracts were combined, washed with brine (×1), dried over MgSO₄ and concentrated in vacuo. The resulting crude residue was purified using flash column chromatography (0-3% MeOH in DCM) to give a clear oil, which was co-evaporated in DCM and diethyl ether to give the title compound as a white powder (79 mg, 83%).

¹H NMR (401 MHz, Chloroform-d) δ 10.90 (br s, 1H), 8.55 (s, 1H), 8.21 (s, 1H), 8.02 (s, 1H), 7.65 (t, J=6.3 Hz, 1H), 7.61-7.49 (m, 4H), 7.50-7.37 (m, 5H), 7.37 (sd, J=2.1 Hz, 1H), 7.19 (br s, 1H), 4.84 (br s, 1H), 4.77 (d, J=6.2 Hz, 2H), 4.37 (app. t, 4H), 3.89 (app. t, 4H), 2.68 (dt, J=14.8, 4.7 Hz, 1H), 2.63-2.50 (m, 1H), 2.43-2.30 (m, 1H), 2.20 (s, 3H), 1.40 (br s, 1H), 1.16 (d, J=6.5 Hz, 3H). LCMS: (Method B) Rf=2.923 min, (ESI) m/z: 641.3 ([M+H]⁺, 100%). HRMS (ESI⁺) calcd for [C₃₇H₃₆N₈O₃+H]⁺641.2983, found 641.3000.

### (S)-N-(4-(1-Acetyl-2-methyl-1,2,3,4-tetrahydroquinolin-6-yl)benzyl)-6-(2-(difluoromethyl)-1H-benzo[d]imidazol-1-yl)-8-morpholinoimidazo[1,2-a]pyrazine-2-carboxamide [Cpd 060]

A mixture of (S)-N-(4-(1-acetyl-2-methyl-1,2,3,4-tetrahydroquinolin-6-yl)benzyl)-6-bromo-8-morpholinoimidazo[1,2-a]pyrazine-2-carboxamide (S17.i[Cpd 010]) (100 mg, 0.17 mmol), 2-(difluoromethyl)benzimidazole (56 mg, 0.33 mmol), Cs₂CO₃ (222 mg, 0.62 mmol), tBuXPhos (7 mg, 0.02 mmol) in dry toluene (8 mL) was degassed with N₂ for 10 min. Pd₂(dba)₃ (16 mg, 0.02 mmol) was then added and the mixture was further degassed for another 5 min. The reaction vessel was then evacuated and backfilled with N₂ three times. The reaction mixture was heated to reflux under N₂ for 24 hr. The reaction mixture was then cooled to r.t and additional portions of Pd₂(dba)₃ (48 mg, 0.05 mmol) and tBuXPhos (21 mg, 0.05 mmol) were added. After heating for another 24 hr, the reaction mixture was diluted with DCM and washed with water (×1), brine (×1), dried over MgSO₄ and concentrated in vacuo. The crude residue was then purified using flash column chromatography (0-5% MeOH in DCM) to give the title compound as an off-white powder (15 mg, 13%) ¹H NMR (401 MHz, Chloroform-d) δ 8.25 (s, 1H), 7.95-7.88 (m, 1H), 7.85 (s, 1H), 7.59 (d with fine splitting, J=8.2 Hz, 2H), 7.53-7.36 (m, 8H), 7.21 (br t, 1H), 4.83 (br t, 1H), 4.77 (d, J=6.2 Hz, 2H), 4.45-4.21 (m, 4H), 3.89-3.78 (m, 4H), 2.75-2.65 (m, 1H), 2.65-2.54 (m, 1H), 2.41-2.33 (m, 1H), 2.20 (s, 3H), 1.41 (br t, 1H), 1.16 (d, J=6.5 Hz, 3H). Minor rotamer exists. LCMS: (Method B) Rf=3.007 min, (ESI) m/z: 691.3 ([M+H]⁺, 100%). HRMS (ESI⁺) calcd for [C₃₈H₃₆F₂N₈O₃+H]⁺691.2951, found 691.2970.

### tert-Butyl (2-(4-bromophenyl)propan-2-yl)carbamate (S9.20a)

To a solution of 2-(4-bromophenyl)propan-2-amine (1.10 g, 5.14 mmol) in DCM (20 mL) was added di-tert-butyl dicarbonate (1.53 g, 7.01 mmol) and TEA (1.42 g, 1.96 mL, 14.03 mmol). The resulting mixture was stirred at r.t o.n. The reaction mixture was then diluted with more DCM, washed with 0.5N HCl solution (×2), 2% NaOH solution (×1), dried over MgSO₄ and concentrated in vacuo. The crude material was then purified using flash column chromatography (0-10% EA in PE) to give the title compound as a white solid (1.06 g, 66%). ¹H NMR (401 MHz, Chloroform-d) δ 7.48-7.39 (m, 2H), 7.31-7.22 (m, 2H, partially overlapped with residual CHCl₃ peak), 4.92 (br s, 1H), 1.59 (s, 6H), 1.51-1.10 (m, 9H).

### tert-Butyl (2-(4-(4,4,5,5-tetramethyl-1,3,2-dioxaborolan-2-yl)phenyl)propan-2-yl)carbamate (S9.20b)

A suspension of tert-butyl (2-(4-bromophenyl)propan-2-yl)carbamate (S9.20a) (1 g, 3.18 mmol), bis(pinacolato)diboron (1.62 g, 6.38 mmol), KOAc (1.25 g, 12.74 mmol) in dry dioxane (15 mL) was degassed with N₂ for 15 min. Pd(dppf)Cl₂ (233 mg, 0.32 mmol) was then added and the suspension was further degassed with N₂ for 5 min. The reaction vessel was evacuated and backfilled with N₂ three times. The reaction mixture was then heated at 90° C. o.n before being diluted with DCM and filtered. The filter pad was rinsed with more DCM and the filtrate was concentrated in vacuo. The resulting crude material was then purified using flash column chromatography (0-10% EA in PE) to give the title compound as a white solid (1.07 g, 93%). ¹H NMR (401 MHz, Chloroform-d) δ 7.76 (d, J=8.0 Hz, 2H), 7.39 (d, J=8.3 Hz, 2H), 4.94 (br t, 1H), 1.61 (s, 6H), 1.47-1.27 (m, 21H).

### tert-Butyl (R)-(1-(4-(4,4,5,5-tetramethyl-1,3,2-dioxaborolan-2-yl)phenyl)ethyl)carbamate (S9.22a)

Refer to the synthesis of tert-butyl (2-(4-(4,4,5,5-tetramethyl-1,3,2-dioxaborolan-2-yl)phenyl)propan-2-yl)carbamate (S9.20b), where the starting material was substituted with tert-butyl (R)-(1-(4-bromophenyl)ethyl)carbamate. Yield: 91%. ¹H NMR (401 MHz, Chloro form-d) δ 7.77 (d, J=8.1 Hz, 2H), 7.30 (d, J=7.6 Hz, 2H), 4.80 (br s, 2H), 1.47-1.35 (m, 12H), 1.33 (s, 12H).

### tert-Butyl (S)-(1-(4-(4,4,5,5-tetramethyl-1,3,2-dioxaborolan-2-yl)phenyl)ethyl)carbamate (S9.21a)

Refer to the synthesis of tert-butyl (2-(4-(4,4,5,5-tetramethyl-1,3,2-dioxaborolan-2-yl)phenyl)propan-2-yl)carbamate (S9.20b), where the starting material was substituted with tert-butyl (S)-(1-(4-bromophenyl)ethyl)carbamate. Yield: 88%. ¹H NMR (401 MHz, Chloro form-d) δ 7.77 (d, J=8.1 Hz, 2H), 7.30 (d, J=7.8 Hz, 2H), 4.79 (br s, 2H), 1.48-1.36 (m, 12H), 1.33 (s, 12H).

### (S)-1-(6-(4-(2-Aminopropan-2-yl)phenyl)-2-methyl-3,4-dihydroquinolin-1(2H)-yl)ethan-1-one (S9.20)

Step 1: A mixture of (S)-1-(6-bromo-2-methyl-3,4-dihydroquinolin-1(2H)-yl)ethan-1-one (S9.6) (400 mg, 1.49 mmol), tert-butyl (2-(4-(4,4,5,5-tetramethyl-1,3,2-dioxaborolan-2-yl)phenyl)propan-2-yl)carbamate (S9.20b) (647 mg, 1.79 mmol), K₂CO₃ (619 mg, 4.48 mmol) in DME/H₂O (4:1, 10 mL) was degassed with N₂ for 15 min. Pd(dppf)Cl₂ (109 mg, 0.15 mmol) was then added and the mixture was further degassed for 15 min. The reaction vessel was then evacuated and backfilled with N₂ three times. The reaction mixture was heated to reflux under N₂ for 4 hr. The mixture was then diluted with water and extracted with DCM (×3). The organic extracts were combined, washed with brine, dried over MgSO₄ and concentrated in vacuo. The resulting residue was purified using flash column chromatography (0-60% EA in PE) to give tert-butyl (S)-(2-(4-(1-acetyl-2-methyl-1,2,3,4-tetrahydroquinolin-6-yl)phenyl)propan-2-yl)carbamate as a thick light yellow gel (Boc-S9.20) (583 mg, 92%). ¹H NMR (401 MHz, Chloroform-d) δ 7.56-7.51 (m, 2H), 7.48-7.44 (m, 2H), 7.42 (dd, J=8.0, 1.6 Hz, 1H), 7.38 (br s, 1H), 7.19 (d, J=12.8 Hz, 1H), 4.97 (br s, 1H), 4.84 (br s, 1H), 2.68 (dt, J=14.5, 5.0 Hz, 1H), 2.65-2.51 (m, 1H), 2.37 (ddt, J=9.9, 7.0, 5.0 Hz, 1H), 2.19 (s, 3H), 1.76 (br s, 1H), 1.66 (s, 6H), 1.40 (br s, 9H). LCMS: (Method B) Rf=3.147 min, (ESI) m/z: 423.2 ([M+H]⁺, 100%). Step 2: To a solution of tert-butyl (S)-(2-(4-(1-acetyl-2-methyl-1,2,3,4-tetrahydroquinolin-6-yl)phenyl)propan-2-yl)carbamate (540 mg, 1.28 mmol) in MeOH was added 4N HCl in dioxane (8 mL). The reaction mixture was then stirred at r.t for 2 hr. The solvent was concentrated in vacuo. The crude material was partitioned between DCM and 10% NaOH solution. The layers were separated and the aqueous layer extracted with DCM (×1). The organic extracts were combined, dried over MgSO₄ and concentrated in vacuo to give the title compound as an off-white solid (383 mg, 93%). ¹H NMR (401 MHz, Chloroform-d) δ 7.62-7.52 (m, 4H), 7.42 (dd, J=8.2, 1.9 Hz, 1H), 7.38 (sd, J=2.0 Hz, 1H), 7.19 (br s, 1H), 4.84 (br s, 1H), 2.67 (td, J=8.9, 8.0, 4.0 Hz, 1H), 2.63-2.50 (m, 1H), 2.49-2.24 (m, 3H), 2.19 (s, 3H), 1.57 (s, 6H), 1.40 (br s, 1H), 1.16 (d, J=6.5 Hz, 3H). LCMS: (Method B) Rf=2.212 min, (ESI) m/z: 306.2 ([M−NH₂]⁺, 100%), 323.2 ([M+H]⁺, 5%).

### 1-((S)-6-(4-((S)-1-Aminoethyl)phenyl)-2-methyl-3,4-dihydroquinolin-1(2H)-yl)ethan-1-one (S9.21)

Refer to the synthesis of (S)-1-(6-(4-(2-aminopropan-2-yl)phenyl)-2-methyl-3,4-dihy-droquinolin-1(2H)-yl)ethan-1-one (S9.20), where the boronic acid was substituted with tert-butyl (S)-(1-(4-(4,4,5,5-tetramethyl-1,3,2-dioxaborolan-2-yl)phenyl)ethyl)carbamate (S9.21a). Yield: 80% over 2 steps. ¹H NMR (401 MHz, Chloroform-d) δ 7.55 (d, J=8.3 Hz, 2H), 7.47-7.38 (m, 3H), 7.38 (d, J=2.1 Hz, 1H), 7.19 (s, 1H), 4.84 (s, 1H), 4.18 (q, J=6.6 Hz, 1H), 2.68 (dt, J=14.8, 5.0 Hz, 1H), 2.58 (ddd, J=15.1, 11.1, 4.9 Hz, 1H), 2.37 (ddt, J=12.8, 7.6, 5.0 Hz, 1H), 2.19 (s, 3H), 1.89 (br s, 2H), 1.44 (d, J=6.6 Hz, 3H), 1.38 (s, 1H), 1.16 (d, J=6.5 Hz, 3H). LCMS: (Method B) Rf=2.205 min, (ESI) m/z: 292.2 ([M−NH₂]⁺, 100%), 309.2 ([M+H]⁺, 5%).

### 1-((S)-6-(4-((R)-1-Aminoethyl)phenyl)-2-methyl-3,4-dihydroquinolin-1(2H)-yl)ethan-1-one (S9.22)

Refer to the synthesis of (S)-1-(6-(4-(2-aminopropan-2-yl)phenyl)-2-methyl-3,4-dihy-droquinolin-1(2H)-yl)ethan-1-one (S9.20), where the boronic acid was substituted with tert-butyl (R)-(1-(4-(4,4,5,5-tetramethyl-1,3,2-dioxaborolan-2-yl)phenyl)ethyl)carbamate (S9.22a). Yield: 79% over 2 steps. ¹H NMR (401 MHz, Chloroform-d) δ 7.55 (d, J=8.3 Hz, 2H), 7.46-7.39 (m, 3H), 7.37 (d, J=2.1 Hz, 1H), 7.19 (br s, 1H), 4.84 (br s, 1H), 4.19 (q, J=6.6 Hz, 1H), 2.68 (dt, J=14.9, 5.1 Hz, 1H), 2.62-2.51 (m, 1H), 2.42-2.30 (m, 2H), 2.24 (br s, 2H), 2.19 (s, 3H), 1.45 (d, J=6.6 Hz, 3H), 1.40 (br s, 1H), 1.16 (d, J=6.5 Hz, 3H). LCMS: (Method B) Rf=2.205 min, (ESI) m/z: 292.2 ([M−NH₂]⁺, 100%), 309.2 ([M+H]⁺, 5%).

### (S)-N-(2-(4-(1-Acetyl-2-methyl-1,2,3,4-tetrahydroquinolin-6-yl)phenyl)propan-2-yl)-6-bromo-8-morpholinoimidazo[1,2-a]pyrazine-2-carboxamide (S17.i[Cpd 061])

A solution of (S)-1-(6-(4-(2-aminopropan-2-yl)phenyl)-2-methyl-3,4-dihydroquinolin-1(2H)-yl)ethan-1-one (S9.20) (100 mg, 0.31 mmol), lithium(I) 6-bromo-8-morpholinoimidazo[1,2-a]pyrazine-2-carboxylate (S1.3) (124 mg, 0.36 mmol), PyClock (344 mg, 0.62 mmol), DIPEA (241 mg/0.33 mL, 1.78 mmol) in DMF (8 mL) was stirred at r.t o.n. The reaction mixture was then poured into 5% Na₂CO₃ solution, the resulting precipitate was filtered and purified using flash column chromatography (0-3% MeOH in DCM) to give the title compound as a white solid (172 mg, 80%). ¹H NMR (401 MHz, Chloroform-d) δ 7.94 (s, 1H), 7.62 (s, 1H), 7.56-7.50 (m, 4H), 7.46 (s, 1H), 7.40 (dd, J=8.2, 2.2 Hz, 1H), 7.36 (sd, J=2.0 Hz, 1H), 7.18 (br s, 1H), 4.84 (br s, 1H), 4.34 (app. t, 4H), 3.92-3.85 (m, 4H), 2.67 (dt, J=14.7, 5.1 Hz, 1H), 2.57 (ddd, J=14.8, 11.0, 4.9 Hz, 1H), 2.42-2.31 (m, 1H), 2.19 (s, 3H), 1.87 (s, 6H), 1.41 (br s, 1H), 1.16 (d, J=6.5 Hz, 3H). LCMS: (Method B) Rf=3.086 min, (ESI) m/z: 630.2, 632.2 ([M+H]⁺, 100%).

### (S)-N-(2-(4-(1-Acetyl-2-methyl-1,2,3,4-tetrahydroquinolin-6-yl)phenyl)propan-2-yl)-6-(2-aminopyrimidin-5-yl)-8-morpholinoimidazo[1,2-a]pyrazine-2-carboxamide [Cpd 061]

A mixture of (S)-N-(2-(4-(1-acetyl-2-methyl-1,2,3,4-tetrahydroquinolin-6-yl)phenyl)propan-2-yl)-6-bromo-8-morpholinoimidazo[1,2-a]pyrazine-2-carboxamide (S17.i[Cpd 061]) (100 mg, 0.16 mmol), 2-aminopyrimidine-5-boronic acid (27 mg, 0.19 mmol), K₂CO₃ (66 mg, 0.48 mmol) in a mixture of DME/H₂O (4:1, 10 mL) was degassed with N₂ for 10 min. Pd(dppf)Cl₂ (12 mg, 0.02 mmol) was then added and the mixture was further degassed for another 10 min. The reaction vessel was then evacuated and backfilled with N₂ three times. The reaction mixture was heated to reflux under N₂ o.n. The mixture was then diluted with 2% NaOH solution and extracted with DCM (×2). The organic extracts were combined, dried over MgSO₄ and concentrated in vacuo. The resulting residue was purified using flash column chromatography (0-6% MeOH in DCM) and gave a light brown semi-solid. The semi-solid was co-evaporated in DCM/PE to give a beige solid, which was triturated with a small amount of cold MeOH (3 mL) to provide the title compound as a white powder (70 mg, 68%). ¹H NMR (401 MHz, DMSO-d₆) δ 8.78 (s, 2H), 8.46 (s, 1H), 8.22 (s, 1H), 8.05 (s, 1H), 7.60 (d, J=8.5 Hz, 2H), 7.52-7.44 (m, 4H), 7.44-7.34 (m, 1H), 6.88 (s, 2H), 4.72-4.57 (m, 1H), 4.29 (app. t, 4H), 3.81 (app. t, 4H), 2.71 (dt, J=15.0, 5.3 Hz, 1H), 2.60-2.51 (m, 1H), 2.35-2.22 (m, 1H), 2.11 (s, 3H), 1.76 (s, 6H), 1.34 (br s, 1H), 1.06 (d, J=6.4 Hz, 3H). LCMS: (Method B) Rf=2.880 min, (ESI) m/z: 646.4 ([M+H]⁺, 100%). HRMS (ESI⁺) calcd for [C₃₆H₃₉N₉O₃+H]⁺646.3249, found 646.3265.

### N-((S)-1-(4-((S)-1-Acetyl-2-methyl-1,2,3,4-tetrahydroquinolin-6-yl)phenyl)ethyl)-6-bromo-8-morpholinoimidazo[1,2-a]pyrazine-2-carboxamide (S17.i[Cpd 062])

A solution of 1-((S)-6-(4-((S)-1-aminoethyl)phenyl)-2-methyl-3,4-dihydroquinolin-1(2H)-yl)ethan-1-one (S9.21) (100 mg, 0.32 mmol), lithium(I) 6-bromo-8-morpholinoimidazo[1,2-a]pyrazine-2-carboxylate (S1.3) (119 mg, 0.37 mmol), PyClock (328 mg, 0.59 mmol), DIPEA (230 mg/0.31 mL, 1.78 mmol) in DMF (8 mL) was stirred at r.t o.n. The reaction mixture was then poured into 5% Na₂CO₃ solution, the resulting precipitate was filtered and purified using flash column chromatography (0-3% MeOH in DCM). Fractions containing the desired product were concentrated and gave a gel, which was co-evaporated in DCM/PE to provide the title compound as a white solid (185 mg, 92%). ¹H NMR (401 MHz, Chloroform-d) δ 8.01 (s, 1H), 7.63 (s, 1H), 7.57 (d with fine splitting, J=8.3 Hz, 2H), 7.47 (d with fine splitting, J=8.3 Hz, 2H), 7.40 (dd, J=8.2, 2.1 Hz, 1H), 7.36 (sd, J=2.1 Hz, 1H), 7.31 (br d, J=8.3 Hz, 1H), 7.20 (br s, 1H), 5.40 (p, J=7.1 Hz, 1H), 4.83 (br s, 1H), 4.31 (app. t, 4H), 3.89-3.80 (m, 4H), 2.68 (dt, J=14.8, 5.0 Hz, 1H), 2.58 (ddd, J=15.1, 11.0, 5.0 Hz, 1H), 2.43-2.30 (m, 1H), 2.19 (s, 3H), 1.67 (d, J=7.0 Hz, 3H), 1.41 (br s, 1H), 1.16 (d, J=6.5 Hz, 3H). LCMS: (Method B) Rf=2.941 min, (ESI) m/z: 617.2, 619.2 ([M+H]⁺, 100%).

### N-((S)-1-(4-((S)-1-Acetyl-2-methyl-1,2,3,4-tetrahydroquinolin-6-yl)phenyl)ethyl)-6-(2-aminopyrimidin-5-yl)-8-morpholinoimidazo[1,2-a]pyrazine-2-carboxamide [Cpd 062]

Refer to the synthesis of (S)-N-(2-(4-(1-acetyl-2-methyl-1,2,3,4-tetrahydroquinolin-6-yl)phenyl)propan-2-yl)-6-(2-aminopyrimidin-5-yl)-8-morpholinoimidazo[1,2-a]pyrazine-2-carboxamide ([Cpd061]), where the halogenated starting material was substituted with N-((S) 1-(4-((S)-1-acetyl-2-methyl-1,2,3,4-tetrahydroquinolin-6-yl)phenyl)ethyl)-6-bromo-8-morpholinoimidazo[1,2-a]pyrazine-2-carboxamide (S17.i[Cpd 062]). Yield: 58%. ¹H NMR (401 MHz, DMSO-d₆) δ 8.78 (s, 2H), 8.61 (d, J=8.5 Hz, 1H), 8.46 (s, 1H), 8.28 (s, 1H), 7.63 (d, J=8.4 Hz, 2H), 7.52-7.45 (m, 4H), 7.44-7.32 (m, 1H), 6.88 (s, 2H), 5.27 (dq, J=7.3 Hz, 1H), 4.72-4.56 (m, 1H), 4.30 (app. t, 4H), 3.79 (app. t, 4H), 2.71 (dt, J=15.2, 5.4 Hz, 1H), 2.55 (dt, J=9.5, 4.8 Hz, 1H), 2.34-2.22 (m, 1H), 2.11 (s, 3H), 1.58 (d, J=7.0 Hz, 3H), 1.34 (br s, 1H), 1.06 (d, J=6.4 Hz, 3H). LCMS: (Method B) Rf=2.738 min, (ESI) m/z: 632.3 ([M+H]⁺, 100%). HRMS (ESI⁺) calcd for [C₃₅H₃₇N₉O₃+H]⁺632.3092, found 632.3108.

### N-((R)-1-(4-((S)-1-Acetyl-2-methyl-1,2,3,4-tetrahydroquinolin-6-yl)phenyl)ethyl)-6-bromo-8-morpholinoimidazo[1,2-a]pyrazine-2-carboxamide (S17.i[Cpd 063])

Refer to the synthesis of N-((S)-1-(4-((S)-1-acetyl-2-methyl-1,2,3,4-tetrahydroquinolin-6-yl)phenyl)ethyl)-6-bromo-8-morpholinoimidazo[1,2-a]pyrazine-2-carboxamide (S17.i[Cpd 062]), where the amine was substituted with 1-((S)-6-(4-((R)-1-aminoethyl)phenyl)-2-methyl-3,4-dihydroquinolin-1(2H)-yl)ethan-1-one (S9.22). Yield: 87%. ¹H NMR (401 MHz, Chloro form-d) δ 8.01 (s, 1H), 7.63 (s, 1H), 7.57 (d with fine splitting, J=8.3 Hz, 2H), 7.47 (d with fine splitting, J=8.3 Hz, 2H), 7.41 (dd, J=8.2, 2.2 Hz, 1H), 7.36 (sd, J=2.1 Hz, 1H), 7.31 (br d, J=8.3 Hz, 1H), 7.20 (br s, 1H), 5.40 (p, J=7.1 Hz, 1H), 4.83 (br s, 1H), 4.31 (br s, 4H), 3.89-3.82 (m, 4H), 2.68 (dt, J=14.8, 5.0 Hz, 1H), 2.58 (ddd, J=15.1, 11.0, 4.8 Hz, 1H), 2.19 (s, 3H), 1.68 (d, J=6.9 Hz, 3H), 1.41 (br s, 1H), 1.16 (d, J=6.4 Hz, 3H). LCMS: (Method B) Rf=3.125 min, (ESI) m/z: 617.2, 619.2 ([M+H]⁺, 100%).

### N-((R)-1-(4-((S)-1-Acetyl-2-methyl-1,2,3,4-tetrahydroquinolin-6-yl)phenyl)ethyl)-6-(2-aminopyrimidin-5-yl)-8-morpholinoimidazo[1,2-a]pyrazine-2-carboxamide [Cpd 063]

Refer to the synthesis of (S)-N-(2-(4-(1-acetyl-2-methyl-1,2,3,4-tetrahydroquinolin-6-yl)phenyl)propan-2-yl)-6-(2-aminopyrimidin-5-yl)-8-morpholinoimidazo[1,2-a]pyrazine-2-carboxamide ([Cpd 061]), where the halogenated starting material was substituted with N-((R) 1-(4-((S)-1-acetyl-2-methyl-1,2,3,4-tetrahydroquinolin-6-yl)phenyl)ethyl)-6-bromo-8-morpholinoimidazo[1,2-a]pyrazine-2-carboxamide (S17.i[Cpd 063]). Yield: 61%. ¹H NMR (401 MHz, DMSO-d₆) δ 8.78 (s, 2H), 8.61 (d, J=8.6 Hz, 1H), 8.45 (s, 1H), 8.28 (s, 1H), 7.63 (d, J=8.3 Hz, 2H), 7.53-7.44 (m, 4H), 7.44-7.33 (m, 1H), 6.88 (s, 2H), 5.27 (dq, J=7.2 Hz, 1H), 4.71-4.57 (m, 1H), 4.30 (app. t, 4H), 3.79 (app. t, 4H), 2.70 (dt, J=15.1, 5.3 Hz, 1H), 2.60-2.52 (m, 1H), 2.33-2.23 (m, 1H), 2.10 (s, 3H), 1.58 (d, J=7.0 Hz, 3H), 1.34 (br s, 1H), 1.05 (d, J=6.4 Hz, 3H). LCMS: (Method B) Rf=2.762 min, (ESI) m/z: 632.4 ([M+H]⁺, 100%). HRMS (ESI⁺) calcd for [C₃₅H₃₇N₉O₃+H]⁺632.3092, found 632.3108.

### Ethyl 6-bromo-8-(piperidin-1-yl)imidazo[1,2-a]pyrazine-2-carboxylate (523.1)

To a solution of ethyl 6,8-dibromoimidazo[1,2-a]pyrazine-2-carboxylate (S1.1) (300 mg, 0.86 mmol) in DCM (8 mL) was added piperidine (220 mg/255 uL, 2.58 mmol). The reaction mixture was stirred at r.t for 1 hr. The mixture was then diluted with DCM and washed with 0.5 M HCl solution (×2). The organic layer was then dried over MgSO₄ and concentrated in vacuo to give the title compound as a light yellow solid (301 mg, 99%). This compound was used directly in the next step without further purification. ¹H NMR (401 MHz, Chloroform-d) δ 7.94 (s, 1H), 7.50 (s, 1H), 4.41 (q, J=7.1 Hz, 3H), 4.31 (br s, 4H), 1.76-1.69 (m, 6H), 1.40 (t, J=7.1 Hz, 4H). LCMS: (Method B) Rf=3.154 min, (ESI) m/z: 353.1, 355.1 ([M+H]⁺, 100%).

### 6-Bromo-8-(piperidin-1-yl)imidazo[1,2-a]pyrazine-2-carboxylic acid (S23.2)

A solution of ethyl 6-bromo-8-(piperidin-1-yl)imidazo[1,2-a]pyrazine-2-carboxylate (S23.1) (300 mg, 0.85 mmol) and LiOH·H₂O (143 mg, 3.40 mmol) in THE/water (4:1, 8 mL) was heated to reflux for 1.5 hr. THE was then removed in vacuo and the remaining aqueous suspension was acidified to pH=1 with 1N HCl solution. The solid precipitate was collected via filtration, washed with water and dried to give the title compound as an off-white solid (255 mg, 92%). ¹H NMR (401 MHz, DMSO-d₆) δ 8.36 (s, 1H), 8.05 (s, 1H), 4.23 (br s, 4H), 1.77-1.52 (m, 6H). LCMS: (Method B) Rf=2.808 min, (ESI) m/z: 325.0, 327.0 ([M+H]⁺, 100%).

### (S)-N-(4-(1-Acetyl-2-methyl-1,2,3,4-tetrahydroquinolin-6-yl)benzyl)-6-bromo-8-(piperi-din-1-yl)imidazo[1,2-a]pyrazine-2-carboxamide (S23.3)

A solution of (S)-1-(6-(4-(aminomethyl)phenyl)-2-methyl-3,4-dihydroquinolin-1(2H)-yl)ethan-1-one (S9.12) (30 mg, 0.10 mmol), 6-bromo-8-(piperidin-1-yl)imidazo[1,2-a]pyrazine-2-carboxylic acid (S23.2) (40 mg, 0.12 mmol), PyClock (114 mg, 0.21 mmol), DIPEA (79 mg/110 uL, 0.61 mmol) in DMF was stirred at r.t o.n. The reaction was quenched by adding into by adding into 5% Na₂CO₃ solution. The resulting precipitate was filtered and dried to give the crude product as a light pink solid (72 mg), which was used directly in the next step without further purification. LCMS: (Method B) Rf=3.165 min, (ESI) m/z: 601.2, 603.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-(piperidin-1-yl)imidazo[1,2-a]pyrazine-2-carboxamide [Cpd 064]

A mixture of crude (S)-N-(4-(1-acetyl-2-methyl-1,2,3,4-tetrahydroquinolin-6-yl)benzyl)-6-bromo-8-(piperidin-1-yl)imidazo[1,2-a]pyrazine-2-carboxamide (S23.3) (72 mg), 2-aminopyrimidine-5-boronic acid (20 mg, 0.14 mmol), K₂CO₃ (50 mg, 0.36 mmol) in dioxane/water (4:1, 5 mL) was degassed with N₂ for 5 min. PdCl₂(PPh₃)₂ (9 mg, 0.01 mmol) was then added and the mixture was degassed for another 5 min. The reaction vessel was then evacuated and backfilled with N₂ three times and the reaction mixture was heated to reflux under N₂ o.n. The mixture was then diluted with 2% NaOH solution and extracted with DCM (×2). The organic extracts were combined, washed with brine, dried over MgSO₄ and concentrated in vacuo. The residue was then purified using preparative TLC (6% MeOH in DCM) to give the title compound as an off-white powder (13 mg, 21% over 2 steps). ¹H NMR (401 MHz, DMSO-d₆) δ 8.94 (br t, J=6.4 Hz, 1H), 8.77 (s, 2H), 8.39 (s, 1H), 8.25 (s, 1H), 7.63 (d, J=8.2 Hz, 2H), 7.54-7.44 (m, 2H), 7.41 (d, J=8.1 Hz, 3H), 6.87 (s, 2H), 4.69-4.60 (m, 1H), 4.54 (d, J=6.3 Hz, 2H), 4.30 (br s, 4H), 2.77-2.65 (m, 1H), 2.59-2.52 (m, 1H), 2.34-2.22 (m, 1H), 2.10 (s, 3H), 1.74-1.59 (m, 6H), 1.34 (br s, 1H), 1.05 (d, J=6.4 Hz, 3H). LCMS: (Method B) Rf=2.802 min, (ESI) m/z: 616.4 ([M+H]⁺, 100%). HRMS (ESI⁺) calcd for [C₃₅H₃₇N₉O₂+H]⁺616.3143, found 616.3163.

### Diethyl 1H-pyrrole-2,4-dicarboxylate (S24.1)

To a solution of ethyl isocyanoacetate (5.0 g, 44.2 mmol) and DBU (8.75 g, 8.6 mL, 57.5 mmol) in dry THE (50 mL) was added portionwise paraformaldehyde (0.8 g, 26.6 mmol) with cooling if necessary. The reaction mixture was then stirred at r.t o.n under N₂ with vigorous stirring. The solvent was then removed in vacuo. The residue was re-dissolved in water and extracted with EA (×2). The organic extracts were combined, washed with water (×2) and brine (×1), dried over MgSO₄ and concentrated in vacuo to give the title compound as a light yellow oil which crystalized on standing (1.8 g, 39%). ¹H NMR (401 MHz, Chloroform-d) δ 9.31 (br s, 1H), 7.53 (dd, J=3.2, 1.5 Hz, 1H), 7.30 (dd, J=2.6, 1.5 Hz, 1H), 4.34 (q, J=7.1 Hz, 2H), 4.30 (q, J=7.1 Hz, 2H), 1.36 (app. q, J=7.2 Hz, 6H). LCMS: (Method B) Rf=2.686 min, (ESI) m/z: 212.1 ([M+H]⁺, 100%).

### Diethyl 1-amino-1H-pyrrole-2,4-dicarboxylate (S24.2)

A suspension of diethyl 1H-pyrrole-2,4-dicarboxylate (S24.1) (1.8 g, 8.5 mmol) in MTBE (50 mL) was cooled to 0° C. To this mixture was added cold 30% NaOH solution (21.5 mL), NH₄C₁ (2.7 g), Aliquat 336 (90 mg) and Conc. ammonia solution (7.5 mL) at the same temperature and stirred for 10 min. To the mixture was then added bleach solution (4%, 120 mL) over 20 min and the reaction mixture was then stirred at 0° C. for 3 hr (the reaction progress was monitored using ¹H NMR). To the reaction mixture was then added diethyl ether. The layers were separated and the aqueous layer extracted again with diethyl ether (×1). The organic extracts were combined, washed with sat. bicarb solution (×1), brine (×1), dried over MgSO₄ and concentrated in vacuo to give the title compound as a light yellow oil (1.6 g, 83%). ¹H NMR (401 MHz, Chloroform-d) δ 7.50 (d, J=2.1 Hz, 1H), 7.25 (d, J=2.1 Hz, 1H), 4.39-4.21 (m, 6H), 1.38-1.31 (m, 8H). LCMS: (Method B) Rf=2.686 min, (ESI) m/z: 227.1 ([M+H]⁺, 100%).

### Diethyl 1-ureido-1H-pyrrole-2,4-dicarboxylate (S24.3); Diethyl 1-(3-carbamoylureido)-1H-pyrrole-2,4-dicarboxylate (Biuret by-product S24.4)

A solution of diethyl 1-amino-1H-pyrrole-2,4-dicarboxylate (S24.2) (1.75 g, 7.74 mmol) in AcOH/water (1:1, 20 mL) was cooled to 0° C. To this cold solution was dropwise added KOCN (1.01 g, 12.45 mmol) in water (5 mL). The reaction mixture was then heated at 50° C. for 1 hr. The reaction mixture was then cooled to 0° C. again and another portion of KOCN (0.63 g, 7.77 mmol) was added. The reaction mixture was heated at 50° C. for another 1 hr. After cooling to r.t, the reaction mixture was diluted with water, the resulting the suspension was cooled to 0° C. and stirred for 20 min. The suspension was then filtered and washed with water to give S24.3 as an off-white powder which was contaminated with the biuret by-product (S24.4) (1.78 g), a ratio of 4:1 between the desired product and by-product was determined by analytical HPLC. The crude product was used in the next step without further purification. LCMS (S24.3): (Method B) Rf=2.417 min, (ESI) m/z: 270.1 ([M+H]⁺, 100%). LCMS (S24.4): (Method B) Rf=2.489 min, (ESI) m/z: 313.1 ([M+H]⁺, 100%). ¹H NMR (524.3) (401 MHz, DMSO-d₆) δ 9.39 (s, 1H), 7.59 (sd, J=2.1 Hz, 1H), 7.09 (sd, J=2.1 Hz, 1H), 6.33 (s, 2H), 4.25-4.16 (m, 4H), 1.30-1.23 (m, 6H).

### Ethyl 2,4-dioxo-1,2,3,4-tetrahydropyrrolo[2,1-f][1,2,4]triazine-6-carboxylate (S24.5)

0.95 g of crude mixture of starting materials (S24.3 and S24.4) from the last step was dissolved in abs. EtOH (45 mL). To this solution was added K₂CO₃ (2.09 g, 15.1 mmol). The reaction mixture was then heated to reflux o.n. A thick suspension was formed and the solvent was then concentrated in vacuo. The resulting solid was suspended in water (5 mL) and acidified to pH=1 with Conc. HCl solution. The solid was then filtered, washed with water and a small amount of cold EtOH (3 mL) to give the title compound as a white powder (610 mg, 66% over 2 steps). ¹H NMR (401 MHz, DMSO-d₆) δ 11.39 (s, 1H), 7.53 (sd, J=1.9 Hz, 1H), 7.05 (sd, J=1.9 Hz, 1H), 4.22 (q, J=7.0 Hz, 2H), 1.27 (t, J=7.1 Hz, 3H). LCMS: (Method B) Rf=2.297 min, (ESI) m/z: 224.1 ([M+H]⁺, 100%).

### Ethyl 2,4-dichloropyrrolo[2,1-f][1,2,4]triazine-6-carboxylate (24.6)

To a suspension of ethyl 2,4-dioxo-1,2,3,4-tetrahydropyrrolo[2,1-f][1,2,4]triazine-6-carboxylate (S24.5) (530 mg, 2.37 mmol) in 4N HCl in dioxane (15 mL) was dropwise added POCl₃ (8.70 mL) and DIPEA (1.74 mL). The mixture was heated at 100° C. for 26 hr. The reaction mixture was then quenching by adding into an ice-water mixture, the resulting precipitate was filtered, washed with water and dried to give title compound as a yellow powder (530 mg, 86%). The crude product was then used directly in the next step without further purification. ¹H NMR (401 MHz, Chloroform-d) δ 8.26 (sd, J=1.6 Hz, 1H), 7.47 (sd, J=1.5 Hz, 1H), 4.40 (q, J=7.1 Hz, 2H), 1.41 (t, J=7.1 Hz, 3H). LCMS: (Method B) Rf=3.186 min, (ESI) m/z: 260.0 ([M+H]⁺, 100%).

### Ethyl 2-chloro-4-morpholinopyrrolo[2,1-f][1,2,4]triazine-6-carboxylate (S24.7)

To a solution of ethyl 2,4-dichloropyrrolo[2,1-f][1,2,4]triazine-6-carboxylate (S24.6) (265 mg, 1.02 mmol) in DCM (10 mL) was added morpholine (356 mg, 353 uL, 4.09 mmol). The reaction mixture was stirred at r.t for 1 hr. The organic then was diluted with more DCM, washed with water (×1), brine (×1), dried over MgSO₄ and concentrated in vacuo. The crude residue was then purified using flash column chromatography (0-10% EA in DCM) to give a light brown solid, which was triturated with PE and yielded the title compound as a white pow der (280 mg, 88%). ¹H NMR (401 MHz, Chloroform-d) δ 7.99 (sd, J=1.6 Hz, 1H), 7.20 (sd, J=1.6 Hz, 1H), 4.35 (q, J=7.1 Hz, 2H), 4.12-4.02 (m, 4H), 3.91-3.79 (m, 4H), 1.38 (t, J=7.1 Hz, 3H). LCMS: (Method B) Rf=2.895 min, (ESI) m/z: 311.1 ([M+H]⁺, 100%).

### 2-Chloro-4-morpholinopyrrolo[2,1-f][1,2,4]triazine-6-carboxylic acid (S24.8)

To a suspension of ethyl 2-chloro-4-morpholinopyrrolo[2,1-f][1,2,4]triazine-6-carboxylate (S24.7) (270 mg, 0.87 mmol) in MeOH/water (1:1, 20 mL) was added LiOH·H₂O (331 mg, 7.88 mmol). The reaction mixture was heated at 50° C. for 1 hr. MeOH was then removed in vacuo. The remaining aqueous residue was acidified with 1N HCl solution to pH=3. The resulting precipitate was filtered, washed with water and dried on air to give the title compound as a white powder (228 mg, 93%). II-1 NMR (401 MHz, DMSO-d₆) δ 8.13 (s, 1H), 7.40 (s, 1H), 4.05-3.91 (m, 4H), 3.75 (app. t, 4H). LCMS: (Method B) Rf=2.547 min, (ESI) m/z: 283.1 ([M+H]⁺, 100%).

### (S)-N-(4-(1-Acetyl-2-methyl-1,2,3,4-tetrahydroquinolin-6-yl)benzyl)-2-chloro-4-morpholinopyrrolo[2,1-f][1,2,4]triazine-6-carboxamide (S24.9)

A solution of 2-chloro-4-morpholinopyrrolo[2,1-f][1,2,4]triazine-6-carboxylic acid (S24.8) (125 mg, 0.44 mmol), (S)-1-(6-(4-(aminomethyl)phenyl)-2-methyl-3,4-dihydroquino-lin-1(2H)-yl)ethan-1-one (S9.12) (100 mg, 0.34 mmol), HCTU (281 mg, 0.68 mmol) and DIPEA (267 mg, 0.32 mL, 2.07 mmol) in DMF (10 mL) was stirred at r.t o.n. The reaction was then quenched by adding into 5% Na₂CO₃ solution. The resulting precipitate was filtered and then purified using flash column chromatography (0-3% MeOH in DCM) to give the title compound as a white powder (166 mg, 88%). ¹H NMR (401 MHz, Chloroform-d) δ 7.93 (s, 1H), 7.53 (d, J=7.7 Hz, 2H), 7.41 (d, J=7.8 Hz, 2H), 7.38-7.32 (m, 2H), 7.20 (s, 1H), 7.16 (br s, 1H), 6.66 (br s, 1H), 4.81 (br s, 1H), 4.66 (d, J=5.3 Hz, 2H), 4.04 (app. t, 4H), 3.82 (app. t, 4H), 2.68 (dt, J=14.8, 5.0 Hz, 1H), 2.63-2.51 (m, 1H), 2.43-2.30 (m, 1H), 2.13 (s, 3H), 1.42 (br s, 1H), 1.15 (d, J=6.4 Hz, 3H). LCMS: (Method B) Rf=2.841 min, (ESI) m/z: 559.2 ([M+H]⁺, 100%).

### (S)-N-(4-(1-Acetyl-2-methyl-1,2,3,4-tetrahydroquinolin-6-yl)benzyl)-2-(2-aminopyrim-idin-5-yl)-4-morpholinopyrrolo[2,1-f][1,2,4]triazine-6-carboxamide [Cpd 065]

A mixture of (S)-N-(4-(1-acetyl-2-methyl-1,2,3,4-tetrahydroquinolin-6-yl)benzyl)-2-chloro-4-morpholinopyrrolo[2,1-f][1,2,4]triazine-6-carboxamide (S24.9) (100 mg, 0.18 mmol), 2-aminopyrimidine-5-boronic acid (125 mg, 0.90 mmol), Na₂CO₃ (190 mg, 1.80 mmol) in toluene/EtOH/water (6:3:1, 20 mL) was degassed with N₂ for 15 min. PdCl₂(PPh₃)₂ (38 mg, 0.05 mmol) was then added and the mixture was further degassed for another 15 min. The reaction vessel was then evacuated and backfilled with N₂ three times. The reaction mixture was heated to reflux under N₂ o.n. The mixture was then diluted with water and extracted with DCM (×3). The organic extracts were combined, washed with brine, dried over MgSO₄ and concentrated in vacuo. The resulting residue was purified using flash column chromatography (0-6% MeOH in DCM) to give a brown solid, which was triturated with MeOH and yielded the title compound as a white powder (58 mg, 52%). II-1 NMR (401 MHz, DMSO-d₆) δ 8.97 (s, 2H), 8.83 (t, J=6.0 Hz, 1H), 8.20 (d, J=1.6 Hz, 1H), 7.65 (d, J=8.3 Hz, 2H), 7.54-7.44 (m, 3H), 7.45-7.35 (m, 3H), 7.11 (s, 2H), 4.70-4.58 (m, 1H), 4.53 (d, J=5.8 Hz, 2H), 4.07 (app. t, J=4.9 Hz, 4H), 3.79 (app. t, J=4.9 Hz, 4H), 2.78-2.64 (m, 1H), 2.60-2.51 (m, 1H), 2.36-2.21 (m, 1H), 2.11 (s, 3H), 1.34 (br s, 1H), 1.06 (d, J=6.4 Hz, 3H). LCMS: (Method B) Rf=2.686 min, (ESI) m/z: 618.3 ([M+H]⁺, 100%). HRMS (ESI⁺) calcd for [C₃₄H₃₅N₉O₃+H]⁺618.2936, found 618.2949.

### Ethyl 2-azidoacrylate (S25.1)

Step 1: A solution of ethyl 2,3-dibromopropanoate (5.00 g, 19.24 mmol) in DMF (30 mL) was cooled to 0° C. NaN₃ (3.76 g, mmol) was added portionwise and the suspension was stirred at r.t o.n. The reaction was then quenched by adding water and extracted with diethyl ether (×3). The organic extracts were combined, washed with brine (×2), dried over MgSO₄ and concentrated in vacuo. Step 2: The residue was re-dissolved in diethyl ether (40 mL) and cooled to 0° C. DBU (3.3 g/3.24 mL, 21. 68 mmol) was then added dropwise and the reaction mixture was stirred at the same temperature for 1.5 hr. The reaction mixture was then diluted with more diethyl ether, washed with water (×2), brine (×1), dried over MgSO₄ and concentrated in vacuo. The rotavap pressure was lowered to 200 mBar (water bath temperature: 30° C.) and kept for 5 min. The crude material was not dried under high vac due to the low boiling point of the product and the title compound was obtained as a yellow liquid, which was contaminated with a small amount of diethyl ether. ¹H NMR integration suggested a molar ratio of 4:1 between the desired product and diethyl ether, which gave an approximately 88% purity by weight (2.22 g, ˜88% pure, 72% over 2 steps). The crude material was then used in the next step without further purification. ¹H NMR (401 MHz, Chloroform-d) δ 5.84 (sd, J=1.4 Hz, 1H), 5.34 (sd, J=1.4 Hz, 1H), 4.30 (q, J=7.1 Hz, 2H), 1.34 (t, J=7.1 Hz, 3H).

### Diethyl 5-methyl-1H-pyrrole-2,4-dicarboxylate (S25.2)

To a solution of ethyl 2-azidoacrylate (S25.1) (2.00 g, ˜88% pure, 12.47 mmol) in dry EtOH (30 mL) was added ethyl acetoacetate (2.42 g/2.35 mL, 18.60 mmol), glacial acetic acid (1.51 g/1.44 mL, 25.15 mmol), Mn(OAc)₃·2H₂O (0.34 g, 1.27 mmol), and 3A MS (5 g). The mixture was degassed with N₂ for 15 min. The reaction vessel was then evacuated and backfilled with N₂ (×3) and the reaction mixture was heated at 50° C. under N₂ for 2 hr. The reaction mixture was then filtered through celite and the filtrate concentrated in vacuo. The residue was re-dissolved in EA, washed with 10% Na₂CO₃ solution (×2), dried over MgSO₄ and concentrated in vacuo. The oily residue was then triturated with PE to give the title compound as a beige powder (1.91 g, 68%), which was used directly in the next step without further purifica tion. ¹H NMR (400 MHz, Chloroform-d) δ 9.40 (br s, 1H), 7.35-7.13 (m, 1H), 4.39-4.22 (m, 4H), 2.57 (d, J=2.0 Hz, 3H), 1.41-1.30 (m, 6H). LCMS: (Method B) Rf=2.812 min, (ESI) m/z: 226.1 ([M+H]⁺, 100%).

### Diethyl 1-amino-5-methyl-1H-pyrrole-2,4-dicarboxylate (S25.3)

A suspension of diethyl 5-methyl-1H-pyrrole-2,4-dicarboxylate (S25.2) (1.75 g, 7.77 mmol) in MTBE (50 mL) was cooled to 0° C. To this mixture was added cold 30% NaOH solution (20.1 mL), NH₄Cl (2.5 g), Aliquat 336 (84 mg) and Conc. ammonia solution (6.9 mL) at the same temperature and stirred for 10 min. To the mixture was then added Bleach solution (4%, 110 mL) over 20 min and the reaction mixture was stirred at 0° C. for 3 hr (the reaction progress was monitored using ¹H NMR). To the reaction mixture was then added diethyl ether. The layers were separated and the aqueous layer extracted again with diethyl ether (×1). The organic extracts were combined, washed with sat. bicarb solution (×1), brine (×1), dried over MgSO₄ and concentrated in vacuo to give the title compound as an off-white solid (1.87 g, quant). ¹H NMR (401 MHz, Chloroform-d) δ 7.26 (s, 1H), 4.30 (q, J=7.1 Hz, 3H), 4.27 (q, J=7.1 Hz, 2H), 1.36 (t, J=7.1 Hz, 3H), 1.35 (t, J=7.1 Hz, 3H), 2.58 (s, 2H). LCMS: (Method B) Rf=2.873 min, (ESI) m/z: 241.1 ([M+H]⁺, 100%).

### Diethyl 5-methyl-1-ureido-1H-pyrrole-2,4-dicarboxylate (S25.4); Diethyl 1-(3-car-bamoylureido)-5-methyl-1H-pyrrole-2,4-dicarboxylate (Biuret by-product S25.5)

A solution of diethyl 1-amino-5-methyl-1H-pyrrole-2,4-dicarboxylate (S25.3) (1.83 g, 7.62 mmol) in AcOH/water (2:1, 20 mL) was cooled to 0° C. To this cold solution was dropwise added KOCN (1.24 g, 15.29 mmol) in water (5 mL). The reaction mixture was then heated at 50° C. for 1 hr. The reaction mixture was then cooled to 0° C. again and another portion of KOCN (1.24 g, 15.29 mmol) was added. The reaction mixture was heated at 50° C. for another 1 hr. After cooling to r.t, the reaction mixture was diluted with water, the resulting the suspension was cooled to 0° C. and stirred for 20 min. The suspension was then filtered and washed with water to give S25.4 as an off-white powder which was contaminated with the biuret by-product (S25.5) (2.24 g), an approximated ratio of 3:1 between the desired product and by-product was determined by analytical HPLC. The crude product was used in the next step without further purification. LCMS (S25.4): (Method B) Rf=2.442 min, (ESI) m/z: 284.1 ([M+H]⁺, 100%). LCMS (S25.5): (Method B) Rf=2.509 min, (ESI) m/z: 327.1 ([M+H]⁺, 100%).

### Ethyl 7-methyl-2,4-dioxo-1,2,3,4-tetrahydropyrrolo[2,1-f][1,2,4]triazine-6-carboxylate (S25.6)

1.35 g of crude mixture of starting materials (S25.4 and S25.5) from the last step was dissolved in abs. EtOH (50 mL). To this solution was added K₂CO₃ (2.65 g, 19.17 mmol). The reaction mixture was then heated to reflux o.n. A thick suspension was formed and the solvent was then concentrated in vacuo. The resulting solid was suspended in water (5 mL) and acidified to pH=1 with Conc. HCl. The solid was then filtered, washed with water and a small amount of cold EtOH (3 mL) to give the title compound as a white powder (966 mg, 75% over 2 steps). ¹H NMR (401 MHz, DMSO-d₆) δ 11.47 (s, 1H), 7.06 (s, 1H), 4.21 (q, J=7.1 Hz, 2H), 2.55 (s, 3H), 1.27 (t, J=7.1 Hz, 3H). LCMS: (Method B) Rf=2.450 min, (ESI) m/z: 238.1 ([M+H]⁺, 100%).

### Ethyl 2,4-dichloro-7-methylpyrrolo[2,1-f][1,2,4]triazine-6-carboxylate (S25.7)

To a suspension of ethyl 7-methyl-2,4-dioxo-1,2,3,4-tetrahydropyrrolo[2,1-f][1,2,4]tri-azine-6-carboxylate (S25.6) (600 mg, 2.53 mmol) in 4N HCl in dioxane (15 mL) was added dropwise POCl₃ (9 mL) and DIPEA (1.8 mL). The reaction mixture was heated at 100° C. for 26 hr. The reaction was then quenched by adding into an ice-water mixture. The resulting precipitate was filtered and washed with cold water and dried to give the title compound a light yellow powder (630 mg, 91%). The crude material was then directly used in the next step with out further purification. ¹H NMR (401 MHz, Chloroform-d) δ 7.48 (s, 1H), 4.39 (q, J=7.1 Hz, 2H), 2.84 (s, 3H), 1.42 (t, J=7.2 Hz, 3H). LCMS: (Method B) Rf=3.255 min, (ESI) m/z: 276.0 ([M+H]⁺, 100%).

### Ethyl 2-chloro-7-methyl-4-morpholinopyrrolo[2,1-f][1,2,4]triazine-6-carboxylate (S25.8)

A solution of ethyl 2,4-dichloro-7-methylpyrrolo[2,1-f][1,2,4]triazine-6-carboxylate (S25.7) (600 mg, 2.19 mmol) in DCM (20 mL) was cooled to 0° C. Morpholine (572 mg, 6.57 mmol) was added dropwise at the same temperature. The reaction mixture was then stirred at r.t for 30 min. The reaction mixture was then diluted with more DCM, washed with water (×1) and brine (×1), dried over MgSO₄ and concentrated in vacuo. The residue was then purified using flash column chromatography (0-10% EA in DCM) and yielded a light yellow solid, which was triturated with PE to give the title compound as a white solid (560 mg, 79%). ¹H NMR (401 MHz, Chloroform-d) δ 7.20 (s, 1H), 4.36 (q, J=7.1 Hz, 2H), 4.11-4.01 (m, 4H), 3.92-3.79 (m, 4H), 2.74 (s, 3H), 1.40 (t, J=7.1 Hz, 3H). LCMS: (Method B) Rf=3.082 min, (ESI) m/z: 325.1 ([M+H]⁺, 100%).

### 2-Chloro-7-methyl-4-morpholinopyrrolo[2,1-f][1,2,4]triazine-6-carboxylic acid (S25.9)

To a suspension of ethyl 2-chloro-7-methyl-4-morpholinopyrrolo[2,1-f][1,2,4]triazine-6-carboxylate (S25.8) (230 mg, 0.71 mmol) in MeOH/water (1:1, 30 mL) was added LiOH·H₂O (276 mg, 6.57 mmol). The reaction mixture was heated at 50° C. for 1 hr. MeOH was then removed in vacuo. The remaining aqueous residue was acidified with 1N HCl solution to pH=3 and extracted with EA (×2). The organic extracts were combined, washed with water (×1), dried over MgSO₄ and concentrated in vacuo to give the title compound as a white powder (205 mg, 98%). ¹H NMR (401 MHz, DMSO-d₆) δ 7.36 (s, 1H), 3.96 (app. t, 4H), 3.79-3.68 (m, 4H), 2.60 (s, 3H). LCMS: (Method B) Rf=2.727 min, (ESI) m/z: 297.1 ([M+H]⁺, 100%).

### (S)-N-(4-(1-Acetyl-2-methyl-1,2,3,4-tetrahydroquinolin-6-yl)benzyl)-2-chloro-7-methyl-4-morpholinopyrrolo[2,1-f][1,2,4]triazine-6-carboxamide (525.10)

A solution of (S)-1-(6-(4-(aminomethyl)phenyl)-2-methyl-3,4-dihydroquinolin-1(2H)-yl)ethan-1-one (S9.12) (100 mg, 0.34 mmol), 2-chloro-7-methyl-4-morpholinopyrrolo[2,1-f][1,2,4]triazine-6-carboxylic acid (S25.9) (112 mg, 0.38 mmol), PyClock (377 mg, 0.68 mmol), DIPEA (264 mg/355 uL, 2.04 mmol) in DMF (15 mL) was stirred r.t o.n. The reaction was quenched by added into 5% Na₂CO₃ solution. The resulting precipitate was filtered and washed with water. The dried solid residue was then purified using flash column chromatography (0-3% MeOH in DCM) and yielded an oil, which was co-evaporated in DCM/diethyl ether to give the title compound as an off-white powder (175 mg, 90%). ¹H NMR (401 MHz, Chloroform-d) δ 7.56 (d, J=8.3 Hz, 2H), 7.43 (d, J=8.2 Hz, 2H), 7.40-7.35 (m, 2H), 7.19 (br s, 1H), 7.01 (s, 1H), 6.33 (br t, J=5.8 Hz, 1H), 4.82 (br s, 1H), 4.67 (d, J=5.4 Hz, 2H), 4.08-3.95 (m, 4H), 3.81 (dd, J=5.7, 4.2 Hz, 4H), 2.75 (s, 3H), 2.69 (dt, J=14.9, 5.0 Hz, 1H), 2.63-2.52 (m, 1H), 2.36 (ddt, J=12.7, 7.2, 5.1 Hz, 1H), 2.17 (s, 3H), 1.41 (br s, 1H), 1.15 (d, J=6.5 Hz, 3H). LCMS: (Method B) Rf=3.004 min, (ESI) m/z: 573.2 ([M+H]⁺, 100%).

### (S)-N-(4-(1-Acetyl-2-methyl-1,2,3,4-tetrahydroquinolin-6-yl)benzyl)-2-(2-aminopyrim-idin-5-yl)-7-methyl-4-morpholinopyrrolo[2,1-f][1,2,4]triazine-6-carboxamide [Cpd 066]

A mixture of (S)-N-(4-(1-acetyl-2-methyl-1,2,3,4-tetrahydroquinolin-6-yl)benzyl)-2-chloro-7-methyl-4-morpholinopyrrolo[2,1-f][1,2,4]triazine-6-carboxamide (525.10) (80 mg, 0.14 mmol), 2-aminopyrimidine-5-boronic acid (40 mg, 0.29 mmol), K₂CO₃ (77 mg, 0.56 mmol) in toluene/EtOH/water (6:3:1, 3 mL) was degassed with N₂ for 5 min. PdCl₂(PPh₃)₂ (20 mg, 0.03 mmol) was then added and the mixture was further degassed for another 5 min. The reaction mixture was then heated at 140° C. under microwave irradiation for 2 hr. The mixture was then diluted with water and extracted with DCM (×3). The organic extracts were combined, washed with brine, dried over MgSO₄ and concentrated in vacuo. The resulting residue was purified using flash column chromatography (0-6% MeOH in DCM) to give a yellow solid, which was triturated with MeOH and yielded the title compound as a white powder (20 mg, 23%). ¹H NMR (401 MHz, DMSO-d₆) δ 9.01 (s, 2H), 8.71 (br t, J=6.0 Hz, 1H), 7.65 (d, J=8.2 Hz, 2H), 7.58 (s, 1H), 7.54-7.45 (m, 2H), 7.41 (app. d, 3H), 7.09 (s, 2H), 4.71-4.58 (m, 1H), 4.52 (d, J=5.9 Hz, 2H), 4.07 (app. t, J=4.8 Hz, 4H), 3.79 (app. t, J=4.8 Hz, 4H), 2.76 (s, 3H), 2.74-2.65 (m, 1H), 2.60-2.52 (m, 1H), 2.35-2.23 (m, 1H), 2.11 (s, 3H), 1.34 (br s, 1H), 1.06 (d, J=6.4 Hz, 3H). LCMS: (Method B) Rf=2.647 min, (ESI) m/z: 632.4 ([M+H]⁺, 100%). HRMS (ESI⁺) calcd for [C₃₅H₃₇N₉O₃+H]⁺632.3092, found 632.3107.

### Bis(4-methoxybenzyl)ammonium chloride (526.1)

Step 1: A solution of 4-methoxybenzylamine (2.00 g, 14.58 mmol) and p-anisaldehyde (2.03 g, 14.91 mmol) in abs. EtOH (40 mL) was heated to reflux. Reaction progress was mon itored by proton NMR. After 6 hr, reaction mixture was cooled to 0° C. and NaBH₄ (667 mg, 17.63 mmol) was added portionwise. The reaction mixture was then stirred at r.t o.n. The reaction was quenched by water and the EtOH was concentrated in vacuo. The resulting residue was re-dissolved in DCM and washed with 10% NaOH solution (×1). The organic was dried over MgSO₄ and concentrated in vacuo to give the crude bis(4-methoxybenzyl)amine as a clear oil. Step 2: The crude oil from last step was re-dissolved in diethyl ether. To this solution was added dropwise 4N HCl in dioxane (10 mL). The resulting precipitate was filtered and washed with diethyl ether to give the title compound a white solid (4.03 g, 94%). ¹H NMR (401 MHz, DMSO-d₆) δ 9.49 (br s, 2H), 7.46 (d, J=8.7 Hz, 4H), 6.97 (d, J=8.8 Hz, 4H), 4.02 (t, J=5.7 Hz, 4H), 3.77 (s, 6H). LCMS: (Method B) Rf=2.307 min, (ESI) m/z: 258.2 ([M+H]⁺, 100%).

### 5-Bromo-N,N-bis(4-methoxybenzyl)pyrimidin-2-amine (S26.2)

To a suspension of 5-bromo-2-chloropyrimidine (2.00 g, 10.34 mmol) and bis(4-meth-oxybenzyl)ammonium chloride (S26.1) (3.19 g, 10.86 mmol) in dry dioxane (25 mL) was added DIPEA (4.01 g/5.4 mL, 31.03 mmol). The reaction vessel was then evacuated and backfilled with N₂ (×3) and the reaction mixture was then heated to reflux under N₂ for 24 hr. The reaction mixture was diluted with EA (200 mL) and washed with 0.5N HCl solution (×2), brine (×1), dried over MgSO₄ and concentrated in vacuo. The crude residue was then purified using a silica plug (5-10% EA in PE) to give the title compound as a very light yellow oil which solidified under vacuum (3.65 g, 85%). ¹H NMR (401 MHz, Chloroform-d) δ 8.33 (s, 2H), 7.15 (d with fine splitting, J=8.6 Hz, 4H), 6.84 (d with fine splitting, J=8.7 Hz, 4H), 4.73 (s, 4H), 3.79 (s, 6H). LCMS: (Method B) Rf=3.543 min, (ESI) m/z: 414.1, 416.1 ([M+H]⁺, 100%).

### 1-(2-(Bis(4-methoxybenzyl)amino)pyrimidin-5-yl)ethan-1-one (S26.3)

To a solution of 5-bromo-N,N-bis(4-methoxybenzyl)pyrimidin-2-amine (S26.2) (3.50 g, 8.45 mmol) in dry degassed DMF (30 mL) was added tributyl(1-ethoxyvinyl)tin (4.58 g/4.28 mL, 12.68 mmol) and PdCl₂(PPh₃)₂ (297 mg, 0.42 mmol). This mixture was further degassed with N₂ for 15 min. The reaction vessel was then evacuated and backfilled with N₂ (×3) and the reaction mixture was then heated at 90° C. under N₂ for 24 hr. The insoluble material was re moved via filtration and the filtrate concentrated in vacuo. The residue was re-dissolved in THF (40 mL) and 1N HCl solution (10 mL) was added. The mixture was stirred vigorously at r.t for 1 hr. THF was then removed in vacuo and the remaining aqueous residue was basified with 10% NaOH to pH=14. EA (50 mL) and water (20 mL) were added and the resulting heterogenous mixture was filtered through celite. The heterogenous filtrate was transferred into a separating funnel. The layers were then separated and the aqueous extracted with EA (×2). The organic extracts were combined, washed with brine (×3), dried over MgSO₄ and concentrated in vacuo. The crude material was then purified using flash column chromatography (0-30% EA in PE) to give the title compound as a light yellow oil which solidified on standing (2.56 g, 80%). ¹H NMR (401 MHz, DMSO-d₆) δ 8.91 (s, 2H), 7.18 (d with fine splitting, J=8.6 Hz, 4H), 6.88 (d with fine splitting, J=8.7 Hz, 4H), 4.79 (s, 4H), 3.72 (s, 6H), 2.47 (s, 3H). LCMS: (Method B) Rf=3.267 min, (ESI) m/z: 378.2 ([M+H]⁺, 100%).

### 1-(2-(Bis(4-methoxybenzyl)amino)pyrimidin-5-yl)-2-bromoethan-1-one (S26.4)

A solution of 1-(2-(bis(4-methoxybenzyl)amino)pyrimidin-5-yl)ethan-1-one (S26.3) (2.00 g, 5.30 mmol) and TEA (1.61 g/2.22 mL, 15.91 mmol) in dry THF (30 mL) was cooled to 0° C. The reaction vessel was evacuated and backfilled with N₂ (×3). To this mixture was then added TMSOTf (3.55 g/2.91 mL, 15.97 mmol) at 0° C. and reaction mixture was stirred at the same temperature for 2 hr. NBS (1.42 g, 7.98 mmol) in dry THF (20 mL) was then added dropwise over 10 min and the reaction mixture was stirred at 0° C. for another 1.5 hr. The reaction was quenched by adding water and stirred for 10 min at r.t. EA was then added and the organic was washed with sat. bicarb (×2), dried over MgSO₄ and concentrated in vacuo to give the title compound as a yellow oil. The crude material was used directly in the next step without further purification. LCMS: (Method B) Rf=3.666 min, (ESI) m/z: 456.1, 458.1 ([M+H]⁺, 100%).

### Diethyl 1-(2-(2-(bis(4-methoxybenzyl)amino)pyrimidin-5-yl)-2-oxoethyl)-1H-pyrazole-3,5-dicarboxylate (S26.5)

The crude bromide (S26.4) (1 eq) from the last step was re-dissolved in acetone (50 mL). To this solution was added K₂CO₃ (2.2 g, 15.92 mmol, 3 eq), diethyl 1H-pyrazole-3,5-dicarboxylate (1.24 g, 5.84 mmol, 1.1 eq). The reaction mixture was stirred at r.t for 24 hr. The solvent was then removed in vacuo. The residue was re-dissolved in EA and washed with water (×1), sat. bicarb (×1), dried over MgSO₄ and concentrated in vacuo. The resulting oily residue was then triturated with diethyl ether and filtered to give the title compound as an off-white powder (2.10 g, 68% over 2 steps). ¹H NMR (401 MHz, Chloroform-d) δ 8.92 (s, 2H), 7.44 (s, 1H), 7.18 (d with fine splitting, J=8.6 Hz, 4H), 6.87 (d with fine splitting, J=8.7 Hz, 4H), 5.96 (s, 2H), 4.85 (s, 4H), 4.43 (q, J=7.1 Hz, 2H), 4.31 (q, J=7.1 Hz, 2H), 3.81 (s, 6H), 1.41 (t, J=7.1 Hz, 3H), 1.35 (t, J=7.1 Hz, 3H). LCMS: (Method B) Rf=3.826 min, (ESI) m/z: 588.2 ([M+H]⁺, 100%).

### Ethyl 6-(2-(bis(4-methoxybenzyl)amino)pyrimidin-5-yl)-4-oxo-4,5-dihydropyrazolo[1,5-a]pyrazine-2-carboxylate (S26.6)

A mixture of diethyl 1-(2-(2-(bis(4-methoxybenzyl)amino)pyrimidin-5-yl)-2-ox-oethyl)-1H-pyrazole-3,5-dicarboxylate (S26.5) (2.0 g, 3.40 mmol) and NH₄OAc (4.0 g, 51.89 mmol) in abs. EtOH (15 mL) was heated at 150° C. under microwave irradiation for 1 hr. The reaction mixture was then cooled to 0° C. The resulting precipitate was then filtered, washed with water and cold EtOH to give the title compound as an off-white powder (1.65 g, 89%). 1H NMR (401 MHz, DMSO-d₆) δ 8.78 (br s, 2H), 8.21 (s, 1H), 7.41 (s, 1H), 7.22 (d with fine splitting, J=8.7 Hz, 4H), 6.90 (d with fine splitting, J=8.7 Hz, 4H), 4.780 (s, 4H), 4.36 (q, J=7.1 Hz, 2H), 3.77 (s, 6H), 1.35 (t, J=7.1 Hz, 3H). LCMS: (Method B) Rf=3.798 min, (ESI) m/z: 541.2 ([M+H]⁺, 100%).

### Ethyl 6-(2-aminopyrimidin-5-yl)-4-oxo-4,5-dihydropyrazolo[1,5-a]pyrazine-2-carboxylate (S26.7)

To a solution of ethyl 6-(2-(bis(4-methoxybenzyl)amino)pyrimidin-5-yl)-4-oxo-4,5-dihydropyrazolo[1,5-a]pyrazine-2-carboxylate (S26.6) (1.65 g, 3.05 mmol) in DCE (30 mL) was added TFA (30 mL) and conc. H₂SO₄ (10 mL). The reaction mixture was stirred at r.t o.n. The solvent was then concentrated in vacuo. The residue was re-dissolved in water and neutralized with 5% NaOH solution. The resulting black precipitate was filtered and washed with water. The crude product was then taken up into DCM/MeOH (1:1, 200 mL), any insoluble was re moved by filtration and the filtrate was concentrated in vacuo. The dark brown solid was then triturated with MeOH to give the crude title product as a dark yellow solid (1.2 g). The crude material was used directly in the next step without further purification. LCMS: (Method B) Rf=2.346 min, (ESI) m/z: 301.1 ([M+H]⁺, 100%).

### Ethyl 6-(2-aminopyrimidin-5-yl)-4-morpholinopyrazolo[1,5-a]pyrazine-2-carboxylate (S26.8)

The crude starting material (S26.7) (1.20 g) from the last step was redissolved in DMF. To this solution was added BOP (2.30 g, 5.20 mmol) and DBU (0.92 g/0.90 mL, 6.04 mmol). The mixture was stirred at r.t for 10 min and morpholine (0.52 g/0.52 mL, 5.97 mmol) was then added. The resulting reaction mixture was stirred at r.t o.n. The reaction was quenched by adding into 5% Na₂CO₃ solution, the resulting precipitate was filtered and triturated with MeOH to give the title compound as a light yellow powder (766 mg, 68% over 2 steps)¹H NMR (401 MHz, DMSO-d₆) δ 8.88 (s, 2H), 8.78 (s, 1H), 7.54 (s, 1H), 6.91 (s, 2H), 4.36 (q, J=7.1 Hz, 2H), 3.87-3.74 (m, 8H), 1.34 (t, J=7.1 Hz, 3H). LCMS: (Method B) Rf=3.679 min, (ESI) m/z: 370.1 ([M+H]⁺, 100%).

### 6-(2-Aminopyrimidin-5-yl)-4-morpholinopyrazolo[1,5-a]pyrazine-2-carboxylic acid (S26.9)

To a solution of ethyl 6-(2-aminopyrimidin-5-yl)-4-morpholinopyrazolo[1,5-a]pyrazine-2-carboxylate (S26.8) (600 mg, 1.62 mmol) in THE/water (5:1, 24 mL) was added LiOH·H₂O (102 mg, 2.43 mmol). The reaction mixture was heated to reflux for 1 hr. The sol vent was then concentrated in vacuo and the remaining aqueous residue was diluted with water and acidified with 1N HCl solution to pH=4. The resulting precipitate was filtered, washed with water and dried to give the title compound as a yellow solid (549 mg, 99%). ¹H NMR (401 MHz, DMSO-d₆) δ 8.89 (s, 2H), 8.72 (s, 1H), 7.44 (s, 1H), 6.89 (s, 2H), 3.85-3.77 (m, 8H). LCMS: (Method B) Rf=2.449 min, (ESI) m/z: 342.1 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-4-morpholinopyrazolo[1,5-a]pyrazine-2-carboxamide [Cpd 067]

A solution of (S)-1-(6-(4-(aminomethyl)phenyl)-2-methyl-3,4-dihydroquinolin-1(2H)-yl)ethan-1-one (S9.12) (100 mg, 0.34 mmol), 6-(2-aminopyrimidin-5-yl)-4-morpholinopyrazolo[1,5-a]pyrazine-2-carboxylic acid (S26.9) (139 mg, 0.41 mmol), EDC.HCl (98 mg, 0.51 mmol), HOBT (69 mg, 0.51 mmol) and DIPEA (264 mg/355 uL, 2.04 mmol) in DMF (10 mL) was stirred at r.t for 1.5 d. The reaction was quenched by adding into 5% Na₂CO₃ solution. The resulting precipitate was filtered and purified using flash column chromatography (0-6% MeOH in DCM) to give the title compound as a light yellow powder (104 mg, 50%). ¹H NMR (401 MHz, Chloroform-d) δ 8.83 (s, 2H), 8.10 (sd, J=0.9 Hz, 1H), 7.57 (d, J=8.3 Hz, 2H), 7.45 (d, J=8.1 Hz, 2H), 7.43-7.36 (m, 3H), 7.32 (sd, J=0.9 Hz, 1H), 7.20 (br s, 1H), 5.41 (s, 2H), 4.84 (br s, 1H), 4.73 (d, J=6.0 Hz, 2H), 3.97-3.84 (m, 8H), 2.69 (dt, J=14.9, 5.1 Hz, 1H), 2.64-2.52 (m, 1H), 2.37 (ddt, J=10.2, 7.8, 5.1 Hz, 1H), 2.19 (s, 3H), 1.41 (br s, 1H), 1.16 (d, J=6.5 Hz, 3H). LCMS: (Method B) Rf=2.694 min, (ESI) m/z: 618.3 ([M+H]⁺, 100%). HRMS (ESI⁺) calcd for [C₃₄H₃₅N₉O₃+H]⁺618.2936, found 618.2947.

### (S)-N-(4-(1-Acetyl-2-methyl-1,2,3,4-tetrahydroquinolin-6-yl)benzyl)-6-(2-aminopyrim-idin-5-yl)-7-chloro-4-morpholinopyrazolo[1,5-a]pyrazine-2-carboxamide [Cpd 068]

A solution of (S)-N-(4-(1-acetyl-2-methyl-1,2,3,4-tetrahydroquinolin-6-yl)benzyl)-6-(2-aminopyrimidin-5-yl)-4-morpholinopyrazolo[1,5-a]pyrazine-2-carboxamide [Cpd 067] (15 mg, 0.02 mmol) and NCS (8 mg, 0.06 mmol) in DCM (4 mL) was stirred at r.t for 48 hr. The reaction mixture was then concentrated in vacuo and purified using preparative TLC (6% MeOH in DCM) to give the title compound as an off-white powder (10 mg, 63%). ¹H NMR (401 MHz, DMSO-d₆) δ 9.09 (br t, J=6.2 Hz, 1H), 8.72 (s, 2H), 7.71-7.60 (m, 3H), 7.55-7.46 (m, 2H), 7.46-7.36 (m, 3H), 7.03 (s, 2H), 4.71-4.60 (m, 1H), 4.57 (d, J=6.2 Hz, 2H), 3.85-3.75 (m, 8H), 2.72 (dt, J=15.0, 5.3 Hz, 1H), 2.57-2.52 (m, 1H), 2.35-2.23 (m, 1H), 2.11 (s, 3H), 1.35 (br s, 1H), 1.06 (d, J=6.4 Hz, 3H). LCMS: (Method B) Rf=2.726 min, (ESI) m/z: 652.3 ([M+H]⁺, 100%). HRMS (ESI⁺) calcd for [C₃₄H₃₄C₁N₉O₃+H]⁺652.2546, found 652.2560.

### Diethyl 1-(2-(2-(bis(4-methoxybenzyl)amino)pyrimidin-5-yl)-2-oxoethyl)-1H-pyrrole-2,4-dicarboxylate (S27.1)

Step 1: A solution of 1-(2-(bis(4-methoxybenzyl)amino)pyrimidin-5-yl)ethan-1-one (S26.3) (1.41 g, 3.74 mmol) and TEA (1.13 g/1.56 mL, 11.17 mmol) in dry THF (20 mL) was cooled to 0° C. The reaction vessel was evacuated and backfilled with N₂ (×3). To this mixture was then added TMSOTf (2.47 g/2.03 mL, 11.11 mmol) at 0° C. and reaction mixture was stirred at the same temperature for 2 hr. NBS (1.00 g, 5.62 mmol) in dry THF (12 mL) was then added dropwise over 10 min and the reaction mixture was stirred at 0° C. for another 1.5 hr. The reaction was quenched by adding water and stirred for 10 min at r.t. EA was then added and the organic was washed with sat. bicarb solution (×2), dried over MgSO₄ and concentrated in vacuo to give the crude bromide (S26.4) as a light yellow oil. Step 2: The bromide was re-dissolved in acetone (40 mL), K₂CO₃ (2.06 g, 14.91 mmol) and diethyl 1H-pyrrole-2,4-dicarboxylate (S24.1) (0.83 g, 3.93 mmol) were added. The resulting suspension was stirred at r.t o.n. The solvent was then concentrated in vacuo and the residue re-dissolved in EA. The organic layer was washed with water (×1), brine (×1), dried over MgSO₄ and concentrated in vacuo. The crude residue was then triturated with diethyl ether to give the title compound as a light beige powder (1.43 g, 65% over 2 steps). This was used directly in the next step without further purification. ¹H NMR (401 MHz, Chloroform-d) δ 8.95 (s, 2H), 7.44 (sd, J=1.8 Hz, 1H), 7.43 (sd, J=1.9 Hz, 1H), 7.18 (d with fine splitting, J=8.6 Hz, 4H), 6.86 (d with fine splitting, J=8.7 Hz, 4H), 5.61 (s, 2H), 4.85 (s, 4H), 4.30 (q, J=7.1 Hz, 2H), 4.22 (q, J=7.1 Hz, 2H), 3.81 (s, 6H), 1.35 (t, J=6.8 Hz, 3H), 1.31 (t, J=6.8 Hz, 3H). LCMS: (Method B) Rf=3.187 min, (ESI) m/z: 587.3 ([M+H]⁺, 100%).

### Ethyl 3-(2-(bis(4-methoxybenzyl)amino)pyrimidin-5-yl)-1-oxo-1,2-dihydropyrrolo[1,2-a]pyrazine-7-carboxylate (S27.2)

To a sealed tube was added diethyl 1-(2-(2-(bis(4-methoxybenzyl)amino)pyrimidin-5-yl)-2-oxoethyl)-1H-pyrrole-2,4-dicarboxylate (S27.1) (1.30 g, 2.22 mmol), NH₄OAc (6.80, 88.22 mmol) and abs. EtOH (40 mL). The thick suspension was heated at 80° C. with vigorous stirring for 48 hr. The suspension was then cooled to 0° C. and stirred for 15 min. The resulting precipitate was filtered, washed with water and cold EtOH to give the title compound as a light yellow solid (0.96 g, 80%). ¹H NMR (401 MHz, DMSO-d₆) δ 11.06 (br s, 1H), 8.64 (s, 2H), 7.95 (sd, J=1.7 Hz, 1H), 7.66 (s, 1H), 7.21-7.14 (m, 5H), 6.88 (d, J=8.6 Hz, 4H), 4.76 (s, 4H), 4.26 (q, J=7.1 Hz, 2H), 3.73 (s, 6H), 1.30 (t, J=7.1 Hz, 3H). LCMS: (Method B) Rf=2.995 min, (ESI) m/z: 540.2 ([M+H]⁺, 100%).

### Ethyl 3-(2-(bis(4-methoxybenzyl)amino)pyrimidin-5-yl)-1-morpholinopyrrolo[1,2-a]pyrazine-7-carboxylate (S27.3)

A solution of ethyl 3-(2-(bis(4-methoxybenzyl)amino)pyrimidin-5-yl)-1-oxo-1,2-dihydropyrrolo[1,2-a]pyrazine-7-carboxylate (S27.2) (450 mg, 0.83 mmol), BOP (477 mg, 1.08 mmol) and DBU (191 mg/187 uL, 1.25 mmol) in DMF (15 mL) was stirred at r.t for 10 min. Morpholine (110 mg/109 uL, 1.26 mmol) was then added and the resulting reaction mixture was stirred at r.t o.n. The reaction was then quenched by adding into 5% Na₂CO₃ solution and the resulting precipitate was filtered and washed with water. The solid was then triturated with DCM and diethyl ether to give the title compound as an off-white powder (460 mg, 91%). ¹H NMR (401 MHz, Chloroform-d) δ 8.87 (s, 2H), 7.83 (sd, J=1.4 Hz, 1H), 7.67 (s, 1H), 7.19 (d, J=8.6 Hz, 4H), 7.11 (s, 1H), 6.85 (d, J=8.6 Hz, 4H), 4.83 (s, 4H), 4.37 (q, J=7.1 Hz, 2H), 3.92-3.83 (m, 8H), 3.80 (s, 6H), 1.40 (t, J=7.1 Hz, 3H). LCMS: (Method C) Rf=4.803 min, (ESI) m/z: 509.3 ([M+H]⁺, 100%).

### Ethyl 3-(2-aminopyrimidin-5-yl)-1-morpholinopyrrolo[1,2-a]pyrazine-7-carboxylate (S27.4)

To a solution of ethyl 3-(2-(bis(4-methoxybenzyl)amino)pyrimidin-5-yl)-1-morpholinopyrrolo[1,2-a]pyrazine-7-carboxylate (S27.3) (430 mg, 0.85 mmol) in DCE (20 mL) was added conc. H₂SO₄ (20 drops) and TFA (10 mL). The reaction mixture was stirred vigorously at r.t o.n. Any volatile organic was then concentrated in vacuo. The residue was re-suspended in water and basified to pH=10 using 10% Na₂CO₃ solution. The resulting precipitate was filtered, washed with water and dried on air. The crude solid material was then triturated with MeOH multiple times to give the title compound as a light yellow powder (231 mg, 89%). This was then used in the next step without further purification. ¹H NMR (401 MHz, DMSO-d₆) δ 8.75 (s, 2H), 8.33 (s, 1H), 8.04 (sd, J=1.5 Hz, 1H), 7.17 (s, 1H), 6.88 (br s, 2H), 4.28 (q, J=7.1 Hz, 2H), 3.80-3.72 (m, 8H), 1.31 (t, J=7.1 Hz, 3H). LCMS: (Method B) Rf=2.582 min, (ESI) m/z: 369.2 ([M+H]⁺, 100%).

### 3-(2-Aminopyrimidin-5-yl)-1-morpholinopyrrolo[1,2-a]pyrazine-7-carboxylic acid (S27.5)

A suspension of ethyl 3-(2-aminopyrimidin-5-yl)-1-morpholinopyrrolo[1,2-a]pyrazine-7-carboxylate (S27.4) (120 mg, 0.33 mmol) and LiOH·H₂O (137 mg, 3.26 mmol) in MeOH/water (1:1, 10 mL) was heated to reflux for 1 hr. MeOH was removed in vacuo and the remaining aqueous residue was diluted with water. Any insoluble material was removed via filtration and filtrate acidified to pH=1 with 1N HCl solution. The resulting precipitate was filtered, washed with water to give the title compound as a yellow solid (106 mg, 96%) ¹H NMR (401 MHz, DMSO-d₆) δ 8.78 (s, 2H), 8.37 (s, 1H), 8.00 (sd, J=1.5 Hz, 1H), 7.15 (s, 1H), 6.94 (br s, 2H), 3.80-3.72 (m, 8H). LCMS: (Method B) Rf=2.394 min, (ESI) m/z: 341.1 ([M+H]⁺, 100%).

### (S)-N-(4-(1-Acetyl-2-methyl-1,2,3,4-tetrahydroquinolin-6-yl)benzyl)-3-(2-aminopyrim-idin-5-yl)-1-morpholinopyrrolo[1,2-a]pyrazine-7-carboxamide [Cpd 069]

A solution of (S)-1-(6-(4-(aminomethyl)phenyl)-2-methyl-3,4-dihydroquinolin-1(2H)-yl)ethan-1-one (S9.12) (40 mg, 0.14 mmol), 3-(2-aminopyrimidin-5-yl)-1-morpholinopyrrolo[1,2-a]pyrazine-7-carboxylic acid (S27.5) (56 mg, 0.16 mmol), PyClock (151 mg, 0.27 mmol) and DIPEA (106 mg/142 uL, 0.82 mmol) in DMF (5 mL) was stirred at r.t o.n. The reaction was then quenched by adding into 5% Na₂CO₃ solution. The resulting precipitate was filtered and purified using flash column chromatography (0-5% MeOH in DCM) to give the title compound as a white powder (50 mg, 60%). ¹H NMR (401 MHz, DMSO-d₆) δ 8.88 (t, J=6.0 Hz, 1H), 8.78 (s, 2H), 8.37 (s, 1H), 8.01 (s, 1H), 7.64 (d, J=7.8 Hz, 2H), 7.54-7.30 (m, 6H), 6.82 (s, 2H), 4.70-4.57 (m, 1H), 4.53 (d, J=5.9 Hz, 2H), 3.87-3.66 (m, 8H), 2.71 (dt, J=14.3, 4.8 Hz, 1H), 2.59-2.50 (m, 1H), 2.34-2.22 (m, 1H), 2.10 (s, 3H), 1.34 (br s, 1H), 1.05 (d, J=6.4 Hz, 3H). LCMS: (Method B) Rf=2.619 min, (ESI) m/z: 617.3 ([M+H]⁺, 100%). HRMS (ESI⁺) calcd for [C₃₅H₃₆N₈O₃+H]⁺617.2983, found 617.2995.

### Ethyl 7-chloro-5-hydroxyimidazo[1,2-c]pyrimidine-2-carboxylate (S28.1); Ethyl 7-bromo-5-hydroxyimidazo[1,2-c]pyrimidine-2-carboxylate (S28.2)

A suspension of 4-amino-2,6-dichlorpyrimidine (2.0 g, 12.2 mmol) and ethyl bromopyruvate (5.2 g/3.3 mL, 26.8 mmol) in glacial acetic acid (20 mL) was heated in a sealed vial at 100-110° C. for 3 hr. The reaction was cooled to 0° C. and diluted with diethyl ether (150 mL), the resulting brown precipitate was filtered and washed with diethyl ether to yield the crude title compound which was contaminated with a small amount of the halogen exchanged bromide by-product (3.06 g) (a ratio of 3:1 was determined by ¹H-NMR between the desired product and the bromide by-product). The crude material was directly used in the next step without further purification. ¹H NMR (S28.1) (401 MHz, DMSO-d₆) δ 8.23 (sd, J=0.8 Hz, 1H), 6.87 (sd, J=0.8 Hz, 1H), 6.11 (s, 1H), 4.29 (q, J=7.1 Hz, 2H), 1.30 (t, J=7.1 Hz, 3H). ¹H NMR (S28.2) (401 MHz, DMSO-d₆) δ 8.22 (sd, J=0.8 Hz, 1H), 6.98 (sd, J=0.8 Hz, 1H), 6.23 (s, 1H), 4.29 (q, J=7.1 Hz, 2H, partially overlapped), 1.30 (t, J=7.1 Hz, 3H, overlapped). LCMS (S28.1): (Method B) Rf=2.397 min, (ESI) m/z: 242.0 ([M+H]⁺, 100%). LCMS (S28.2): (Method B) Rf=2.397 min, (ESI) m/z: 286.0, 288.0 ([M+H]⁺, 100%). Analytical HPLC (S28.1): Rf=3.405 min. Analytical HPLC (S28.2): Rf=3.501 min.

### Ethyl 5,7-dichloroimidazo[1,2-c]pyrimidine-2-carboxylate and ethyl 7-bromo-5-chloroimidazo[1,2-c]pyrimidine-2-carboxylate (S28.3)

2.6 g of crude mixture of starting materials (S28.1 and S28.2) from the last step was suspended in POCl₃ (39 mL) and cooled to 0° C. DIPEA (7.8 mL) was dropwise added at the same temperature. The reaction mixture was heated at 70-80° C. o.n. The reaction was then quenched by adding into an ice-water mixture and resulting black precipitate was filtered, washed with water and dried. The solid was re-dissolved in DCM and any insoluble material was removed via filtration. The organic was concentrated in vacuo to give the first batch of crude title compounds as a dark brown solid. The aqueous filtrate was extricated with DCM (×1), dried over MgSO₄ and concentrated in vacuo to give another batch of the crude title compounds. A total of 1.45 g of the crude product was yielded (a ratio of 3:1 between the desired product and the bromide by-product remained unchanged), this was used in the next step with out further purification. ¹H NMR (—Cl) (401 MHz, Chloroform-d) δ 8.29 (sd, J=0.8 Hz, 1H), 7.60 (sd, J=0.8 Hz, 1H), 4.48 (q, J=7.2 Hz, 2H), 1.45 (t, J=7.2 Hz, 3H). ¹H NMR (—Br) (401 MHz, Chloroform-d) δ 8.29 (sd, J=0.9 Hz, 1H, partially overlapped), 7.78 (sd, J=0.9 Hz, 1H), 4.48 (q, J=7.2 Hz, 2H, overlapped), 1.45 (t, J=7.2 Hz, 3H, overlapped). LCMS (—Cl): (Method B) Rf=2.726 min, (ESI) m/z: 260.0 ([M+H]⁺, 100%). LCMS (—Br): (Method B) Rf=2.726 min, (ESI) m/z: 303.9, 306.0 ([M+H]⁺, 100%). Analytical HPLC (—Cl): Rf=4.338 min. Analytical HPLC (—Br): Rf=4.455 min.

### Ethyl 7-chloro-5-morpholinoimidazo[1,2-c]pyrimidine-2-carboxylate and ethyl 7-bromo-5-morpholinoimidazo[1,2-c]pyrimidine-2-carboxylate (S28.4)

1.4 g of the crude mixture of starting materials (S28.3) from the last step was dissolved in DCM (30 mL) and cooled to 0° C. Morpholine (1.88 g/1.86 mL, 21.58 mmol) was added at the same temperature and the reaction mixture was stirred at r.t for 30 min. The reaction mixture was then diluted with more DCM, washed with sat. bicarb solution (×2), dried over MgSO₄ and concentrated in vacuo. The crude residue was then purified using flash column chromatography (0-50% EA in DCM) and a brown oil was yielded. This product was triturated with diethyl ether to give the title compounds as a light yellow solid (1.03 g) (a ratio of 3:1 between the desired product and the bromide by-product remained unchanged). ¹H NMR (—Cl) (401 MHz, Chloroform-d) δ 8.00 (sd, J=0.9 Hz, 1H), 7.26 (sd, J=0.9 Hz, 1H), 4.47 (q, J=7.2 Hz, 2H), 3.96-3.88 (m, 4H), 3.61-3.52 (m, 4H), 1.44 (t, J=7.1 Hz, 3H). ¹H NMR (—Br) (401 MHz, Chloroform-d) δ 7.99 (sd, J=0.9 Hz, 1H), 7.44 (sd, J=0.8 Hz, 1H), 4.47 (q, J=7.2 Hz, 2H, overlapped), 3.96-3.88 (m, 4H, overlapped), 3.61-3.52 (m, 4H, overlapped), 1.44 (t, J=7.1 Hz, 3H, overlapped). LCMS (—Cl): (Method B) Rf=2.600 min, (ESI) m/z: 311.1 ([M+H]⁺, 100%). LCMS (—Br): (Method B) Rf=2.600 min, (ESI) m/z: 355.0, 357.0 ([M+H]⁺, 100%). Analytical HPLC (—Cl): Rf=5.402 min. Analytical HPLC (—Br): Rf=5.523 min.

### 7-Chloro-5-morpholinoimidazo[1,2-c]pyrimidine-2-carboxylic acid and 7-Bromo-5-morpholinoimidazo[1,2-c]pyrimidine-2-carboxylic acid (S28.5)

To the mixture of starting materials (S28.4) (250 mg) in THE/water (3:1, 16 mL) was added 30% NaOH solution (324 uL). The reaction mixture was stirred at r.t for 40 min. THE was removed in vacuo. The remaining aqueous residue was acidified with 1N HCl solution to pH=3, the resulting precipitate was filtered to give the title compounds as a white powder (216 mg) (a ratio of 3:1 between the desired product and the bromide by-product remained un changed). This was used directly in the next step without further purification. ¹H NMR (—Cl) (401 MHz, Methanol-d₄) δ 8.32 (s, 1H), 7.21 (s, 1H), 3.91-3.86 (m, 4H), 3.66-3.62 (m, 4H).

¹H NMR (—Br) (401 MHz, Methanol-d₄) δ 8.31 (s, 1H), 7.39 (s, 1H), 3.91-3.86 (m, 4H, over lapped), 3.66-3.62 (m, 4H, overlapped). LCMS (—Cl): (Method C) Rf=2.940 min, (ESI) m/z: 283.1 ([M+H]⁺, 100%). LCMS (—Br): (Method C) Rf=2.985 min, (ESI) m/z: 327.0, 329.0 ([M+H]⁺, 100%). Analytical HPLC (—Cl): Rf=4.303 min. Analytical HPLC (—Br): Rf=4.561 min.

### (S)-N-(4-(1-Acetyl-2-methyl-1,2,3,4-tetrahydroquinolin-6-yl)benzyl)-7-chloro-5-morpholinoimidazo[1,2-c]pyrimidine-2-carboxamide and (S)-N-(4-(1-Acetyl-2-methyl-1,2,3,4-tet-rahydroquinolin-6-yl)benzyl)-7-bromo-5-morpholinoimidazo[1,2-c]pyrimidine-2-carboxamide (S28.6)

A solution of the mixture of starting materials (S28.5) from the last step (140 mg), (S)-1-(6-(4-(aminomethyl)phenyl)-2-methyl-3,4-dihydroquinolin-1(2H)-yl)ethan-1-one (S9.12) (100 mg, 0.34 mmol), PyClock (377 mg, 0.68 mmol), DIPEA (264 mg/355 uL, 2.04 mmol) in DMF (15 mL) was stirred at r.t o.n. The reaction was quenched by adding into 5% Na₂CO₃ solution, the resulting precipitate was filtered purified using flash column chromatography (0-3% MeOH in DCM) to give the title compounds as a white solid (110 mg) (a ratio of 3:1 be tween the desired product and the bromide by-product remained unchanged). ¹H NMR (—Cl) (401 MHz, Chloroform-d) δ 8.02 (sd, J=0.9 Hz, 1H), 7.75 (br t, J=5.9 Hz, 1H), 7.56 (d, J=8.3 Hz, 2H), 7.46-7.39 (m, 3H), 7.37 (sd, J=2.1 Hz, 1H), 7.19 (br s, 1H), 7.10 (sd, J=0.8 Hz, 1H), 4.84 (br s, 1H), 4.70 (d, J=6.1 Hz, 2H), 3.95-3.86 (m, 4H), 3.62-3.51 (m, 4H), 2.68 (dt, J=14.8, 5.0 Hz, 1H), 2.58 (ddd, J=15.0, 10.8, 4.9 Hz, 1H), 2.37 (ddt, J=10.1, 7.5, 5.1 Hz, 1H), 2.19 (s, 3H), 1.41 (br s, 1H), 1.16 (d, J=6.5 Hz, 3H). ¹H NMR (—Cl) (401 MHz, Chloroform-d) δ 8.01 (sd, J=0.9 Hz, 1H), 7.75 (br t, J=5.9 Hz, 1H), 7.56 (d, J=8.3 Hz, 2H), 7.46-7.39 (m, 3H), 7.37 (sd, J=2.1 Hz, 1H), 7.29 (sd, J=0.8 Hz, 1H), 7.19 (br s, 1H), 4.84 (br s, 1H), 4.70 (d, J=6.1 Hz, 2H), 3.95-3.86 (m, 4H), 3.62-3.51 (m, 4H), 2.68 (dt, J=14.8, 5.0 Hz, 1H), 2.58 (ddd, J=15.0, 10.8, 4.9 Hz, 1H), 2.37 (ddt, J=10.1, 7.5, 5.1 Hz, 1H), 2.19 (s, 3H), 1.41 (br s, 1H), 1.16 (d, J=6.5 Hz, 3H). All signals were overlapped perfectly except for two pairs of signals highlighted. LCMS (—Cl): (Method B) Rf=2.874 min, (ESI) m/z: 559.3 ([M+H]⁺, 100%). LCMS (—Br): (Method B) Rf=2.874 min, (ESI) m/z: 603.2, 605.2 ([M+H]⁺, 100%). Analytical HPLC (—Cl): Rf=6.708 min. Analytical HPLC (—Br): Rf=6.708 min. Two peaks were heavily overlapped, the difference could only be told by the asymmetrical shape of the peak.

### (S)-N-(4-(1-Acetyl-2-methyl-1,2,3,4-tetrahydroquinolin-6-yl)benzyl)-7-(2-aminopyrim-idin-5-yl)-5-morpholinoimidazo[1,2-c]pyrimidine-2-carboxamide [Cpd 070]

A solution of the mixture of starting materials (S28.6) from the last step (80 mg), 2-aminopyrimidine-5-boronic acid (40 mg, 0.29 mmol), K₂CO₃ (119 mg, 0.86 mmol) in DME/water (4:1, 3 mL) was degassed with N₂ for 5 min. PdCl₂(dppf) (16 mg, 0.02 mmol) was then added and the mixture was further degassed for another 5 min. The reaction mixture was then heated at 120-130° C. under microwave irradiation for 2 hr. The mixture was then diluted with water and extracted with DCM (×2). The organic extracts were combined, washed with brine, dried over MgSO₄ and concentrated in vacuo. The resulting residue was purified using flash column chromatography (0-6% MeOH in DCM) to give a yellow solid, which was triturated with MeOH and yielded the title compound as a white powder (19 mg). ¹H NMR (401 MHz, DMSO-d₆) δ 9.03 (br t, J=6.3 Hz, 1H), 9.00 (s, 2H), 8.21-8.13 (m, 1H), 7.69-7.57 (m, 3H), 7.52-7.45 (m, 2H), 7.41 (app. d, 3H), 7.04 (s, 2H), 4.71-4.57 (m, 1H), 4.53 (d, J=6.3 Hz, 2H), 3.84 (app. t, 4H), 3.59-3.46 (m, 4H), 2.71 (dt, J=14.8, 5.2 Hz, 1H), 2.59-2.52 (m, 1H), 2.36-2.21 (m, 1H), 2.10 (s, 3H), 1.34 (br s, 1H), 1.05 (d, J=6.4 Hz, 3H). LCMS: (Method C) Rf=3.375 min, (ESI) m/z: 618.3 ([M+H]⁺, 100%). HRMS (ESI⁺) calcd for [C₃₄H₃₅N₉O₃+H]⁺618.2936, found 618.2950.

### 4-(2-Chloro-9H-purin-6-yl)morpholine (29.1)

To a suspension of 2,6-dichloropurine (2.00 g, 10.58 mmol) in MeOH was added dropwise morpholine (2.04 g/2.02 mL, 23.42 mmol) at 0° C. The reaction mixture was then stirred at r.t for 2 hr. This thick suspension was cooled to 0° C. again and stirred for 15 min. The solid was collected via filtration and washed with MeOH to give the title compound as a white powder (2.51 g, 99%). ¹H NMR (401 MHz, DMSO-d₆) δ 8.15 (s, 1H), 4.18 (br s, 4H), 3.77-3.65 (m, 4H). LCMS: (Method C) Rf=3.034 min, (ESI) m/z: 240.1 ([M+H]⁺, 100%).

### 4-(2-Chloro-9-methyl-9H-purin-6-yl)morpholine (S29.2)

To a suspension of 4-(2-chloro-9H-purin-6-yl)morpholine (S29.1) (1.00 g, 4.18 mmol) and Cs₂CO₃ (2.72 g, 8.25 mmol) in ACN (30 mL) was added iodomethane (0.89 g, 0.39 mL). The reaction mixture was stirred at r.t o.n. The reaction mixture was diluted with DCM, washed with water (×1), dried over MgSO₄ and concentrated in vacuo. The crude solid was then triturated with diethyl ether to give the title compound as a white powder (1.01 g, 95%). ¹H NMR (401 MHz, Chloroform-d) δ 7.67 (s, 1H), 4.29 (br s, 4H), 3.86-3.80 (m, 4H), 3.78 (s, 3H). LCMS: (Method C) Rf=2.554 min, (ESI) m/z: 254.1 ([M+H]⁺, 100%).

### Ethyl 2-chloro-9-methyl-6-morpholino-9H-purine-8-carboxylate (S29.3); Bis(2-chloro-9-methyl-6-morpholino-9H-purin-8-yl)methanone (S29.4)

To a solution of 4-(2-chloro-9-methyl-9H-purin-6-yl)morpholine (S29.2) (400 mg, 1.58 mmol) in dry THF (20 mL) was added TMEDA (275 mg/355 uL, 2.37 mmol). The reaction vessel was evacuated and backfilled with N₂ (×3) and cooled to −78° C. n-BuLi (2.5 M in hexanes, 0.95 mL, 2.38 mmol) was added dropwise at the same temperature. The solution was then warmed up to −40° C. and stirred for 1.5 hr before cooling to −78° C. again. To this mixture was then added ethyl chloroformate (685 mg/0.60 mL, 6.31 mmol) was added quickly with vigorous stirring and the reaction mixture was stirred at −78° C. for another 30 min. The reaction was then quenched by adding into an ice/0.5N HCl solution mixture and extracted with DCM (×2). The organic extracts were combined, washed with water (×1), dried over MgSO₄ and concentrated in vacuo to give a mixture of the desired product and the ketone dimer bis(2-chloro-9-methyl-6-morpholino-9H-purin-8-yl)methanone. This was used directly in the next step without further purification. Desired product LCMS (S29.3): (Method B) Rf=2.825 min, (ESI) m/z: 326.1 ([M+H]⁺, 100%). LCMS (S29.4): (Method B) Rf=3.048 min, (ESI) m/z: 533.1 ([M+H]⁺, 5%), 579.2 ([M+H+2Na]⁺, 100%).

### 2-Chloro-9-methyl-6-morpholino-9H-purine-8-carboxylic acid (S29.5)

The crude mixture (S29.3 and S29.4) from the last step was re-dissolved in THF/water (4:1, 15 mL). LiOH·H₂O (440 mg, 10.48 mmol) was added and the reaction mixture was heated to reflux o.n. THF was then removed in vacuo and the remaining aqueous residue was diluted with 5% NaOH solution. Any insoluble material was removed via filtration and the filtrate extracted with DCM (×3) to remove by-products. The aqueous layer was then acidified with conc. HCl solution to pH=1, the resulting precipitate was filtered and washed with water to give the title compound as a white solid (301 mg, 64% over 2 steps). ¹H NMR (401 MHz, DMSO-d₆) δ 4.53 (br s, 2H), 4.20-3.61 (m, 2H, very broad signal overlapped with the other peaks), 3.90 (s, 3H), 3.81-3.66 (m, 4H). LCMS: (Method B) Rf=2.509 min, (ESI) m/z: 298.0 ([M+H]⁺, 100%).

### (S)-N-(4-(1-Acetyl-2-methyl-1,2,3,4-tetrahydroquinolin-6-yl)benzyl)-2-chloro-9-methyl-6-morpholino-9H-purine-8-carboxamide (S29.6)

A solution of (S)-1-(6-(4-(aminomethyl)phenyl)-2-methyl-3,4-dihydroquinolin-1(2H)-yl)ethan-1-one (S9.12) (90 mg, 0.31 mmol), 2-chloro-9-methyl-6-morpholino-9H-purine-8-carboxylic acid (S29.5) (110 mg, 0.37 mmol), PyClock (340 mg, 0.61 mmol), DIPEA (238 mg/320 uL, 1.84 mmol) in DMF (15 mL) was stirred at r.t o.n. The reaction was then quenched by adding into 5% Na₂CO₃ solution, the resulting precipitate was filtered and purified using flash column chromatography (0-3% MeOH in DCM) to give the title compound as a light yellow solid (170 mg, 97%). ¹H NMR (401 MHz, Chloroform-d) δ 7.71 (br t, J=6.2 Hz, 1H), 7.59 (d with fine splitting, J=8.2 Hz, 2H), 7.47-7.39 (m, 3H), 7.37 (sd, J=1.9 Hz, 1H), 7.22 (br s, 1H), 4.83 (br s, 1H), 4.70 (d, J=6.3 Hz, 2H), 4.59-3.94 (br s, 4H), 4.16 (s, 3H), 3.89-3.75 (m, 4H), 2.69 (dt, J=14.8, 5.2 Hz, 1H), 2.64-2.53 (m, 1H), 2.43-2.32 (m, 1H), 2.19 (s, 3H), 1.41 (br s, 1H), 1.16 (d, J=6.5 Hz, 3H). LCMS: (Method B) Rf=3.032 min, (ESI) m/z: 574.2 ([M+H]⁺, 100%).

### (S)-N-(4-(1-Acetyl-2-methyl-1,2,3,4-tetrahydroquinolin-6-yl)benzyl)-2-(2-aminopyrim-idin-5-yl)-9-methyl-6-morpholino-9H-purine-8-carboxamide [Cpd 071]

A mixture of ((S)-N-(4-(1-acetyl-2-methyl-1,2,3,4-tetrahydroquinolin-6-yl)benzyl)-2-chloro-9-methyl-6-morpholino-9H-purine-8-carboxamide (S29.6) (150 mg, 0.26 mmol), 2-aminopyrimidine-5-boronic acid (58 mg, 0.42 mmol), K₂CO₃ (154 mg, 1.11 mmol) in dioxane/water (4:1, 10 mL) was degassed with N₂ for 15 min. PdCl₂(dppf) (40 mg, 0.05 mmol) was then added and the mixture was degassed for another 15 min. The reaction vessel was then evacuated and backfilled with N₂ three times and the reaction mixture was heated to reflux under N₂ o.n. The mixture was then diluted with water and extracted with DCM (×3). The organic extracts were combined, washed with brine, dried over MgSO₄ and concentrated in vacuo. The resulting residue was purified using flash column chromatography (0-6% MeOH in DCM) to give a dark yellow solid, which was triturated with MeOH and yielded the title compound as a light yellow powder (112 mg, 68%). ¹H NMR (401 MHz, DMSO-d₆) δ 9.38 (br t, J=6.4 Hz, 1H), 9.13 (s, 2H), 7.64 (d with fine splitting, J=8.3 Hz, 2H), 7.51-7.34 (m, 5H), 7.11 (s, 2H), 4.69-4.59 (m, 1H), 4.54 (d, J=6.4 Hz, 2H), 4.30 (br s, 4H), 4.03 (s, 3H), 3.82-3.69 (m, 4H), 2.70 (dt, J=14.9, 5.2 Hz, 1H), 2.59-2.51 (m, 1H), 2.34-2.20 (m, 1H), 2.10 (s, 3H), 1.33 (br s, 1H), 1.05 (d, J=6.4 Hz, 3H). LCMS: (Method B) Rf=2.710 min, (ESI) m/z: 633.4 ([M+H]⁺, 100%). HRMS (ESI⁺) calcd for [C₃₄H₃₆N₁₀O₃+H]⁺633.3045, found 633.3058.

### 1,2-Diamino-5-bromo-3-morpholinopyrazin-1-ium 2,4,6-trimethylbenzenesulfonate (S30.1)

Step 1: tert-butyl ((mesitylsulfonyl)oxy)carbamate (792 mg, 2.51 mmol) was added portionwise to ice-cold TFA (4 mL) and reaction mixture was then stirred at 0° C. for 1.5 hr. To this mixture was then added crushed ice and stirred vigorously at 0° C. Cold water was added to dilute the thick suspension and the white precipitate was filtered and washed with cold water. The wet solid was then re-dissolved with cold DCM, dried over MgSO₄ to prepare a DCM solution of O-(Mesitylenesulfonyl)hydroxylamine (MSH). (Note: dry MSH is potentially ex plosive, do not leave the MSH over-dry during filtration. Storage of the DCM solution is also not recommended even in the freezer) Step 2: To the cold DCM solution of MSH was added 5-bromo-3-morpholinopyrazin-2-amine (S14.1) (500 mg, 1.93 mmol) and reaction mixture was stirred at 0° C. for 30 min before warming up to r.t o.n. The resulting precipitate was then filtered and washed with DCM to give the title compound as a light yellow powder (890 mg, 97%). ¹H NMR (401 MHz, DMSO-d₆) δ 8.66 (br s, 2H), 7.94 (s, 1H), 7.03 (s, 2H), 6.74 (s, 2H), 3.79-3.73 (m, 4H), 3.26-3.19 (m, 4H), 2.49 (s, 6H), 2.17 (s, 3H). LCMS: (Method B) Rf=1.724 min, (ESI) m/z: 274.0, 276.0 ([M+H]⁺, 100%).

### Ethyl 6-bromo-8-morpholino-[1,2,4]triazolo[1,5-a]pyrazine-2-carboxylate (S30.2)

To a solution of 1,2-diamino-5-bromo-3-morpholinopyrazin-1-ium 2,4,6-trimethylbenzenesulfonate (S30.1) (860 mg, 1.81 mmol) in pyridine (8 mL) was added dropwise ethyl chlorooxoacetate (495 mg/405 uL, 3.63 mmol). The reaction mixture was heated at 100° C. for 18 hr. Pyridine was then removed in vacuo and sat. bicarb solution was added slowly to quench the residual acid at 0° C. The aqueous mixture was then extracted with DCM (×2), dried over MgSO₄ and concentrated in vacuo. The resulting crude material was then purified using flash column chromatography (0-15% EA in DCM) to give the title compound as a white powder (575 mg, 89%). ¹H NMR (401 MHz, Chloroform-d) δ 8.04 (s, 1H), 4.53 (q, J=7.1 Hz, 2H), 4.34 (br s, 4H), 3.88-3.81 (m, 4H), 1.47 (t, J=7.1 Hz, 3H). LCMS: (Method B) Rf=2.824 min, (ESI) m/z: 328.0, 330.0 ([M+H]⁺, 100%).

### 6-Bromo-8-morpholino-[1,2,4]triazolo[1,5-a]pyrazine-2-carboxylic acid (S30.3)

To a solution of ethyl 6-bromo-8-morpholino-[1,2,4]triazolo[1,5-a]pyrazine-2-carboxylate (S30.2) (250 mg, 0.71 mmol) in THE/water (4:1, 10 mL) was added LiOH·H₂O (60 mg, 1.43 mmol). The reaction mixture was then heated to reflux for 1 hr. THE was removed in vacuo and the remaining aqueous residue was acidified to pH=1 with 1N HCl solution. The resulting precipitate was filtered and washed with water to give the title compound as a white solid (209 mg, 91%). ¹H NMR (401 MHz, DMSO-d₆) δ 8.63 (s, 1H), 4.18 (br s, 4H), 3.80-3.73 (m, 5H). LCMS: (Method B) Rf=2.512 min, (ESI) m/z: 328.0, 330.0 ([M+H]⁺, 100%).

### (S)-N-(4-(1-Acetyl-2-methyl-1,2,3,4-tetrahydroquinolin-6-yl)benzyl)-6-bromo-8-morpholino-[1,2,4]triazolo[1,5-a]pyrazine-2-carboxamide (S30.4)

A solution of (S)-1-(6-(4-(aminomethyl)phenyl)-2-methyl-3,4-dihydroquinolin-1(2H)-yl)ethan-1-one (S9.12) (90 mg, 0.31 mmol), 6-bromo-8-morpholino-[1,2,4]triazolo[1,5-a]pyrazine-2-carboxylic acid (S30.3) (120 mg, 0.37 mmol), PyClock (340 mg, 0.61 mmol), DIPEA (238 mg/320 uL, 1.84 mmol) in DMF (15 mL) was stirred at r.t o.n. The reaction was then quenched by adding into 5% Na₂CO₃ solution, the resulting precipitate was filtered and purified using flash column chromatography (0-3% MeOH in DCM). Fractions containing desired product were concentrated and the oily residue was triturated with diethyl ether to give the title compound as a white powder (155 mg, 84%). ¹H NMR (401 MHz, Chloroform-d) δ 8.03 (s, 1H), 7.58 (d with fine splitting, J=8.3 Hz, 2H), 7.51-7.43 (m, 3H), 7.41 (dd, J=8.2, 2.2 Hz, 1H), 7.37 (sd, J=2.0 Hz, 1H), 7.20 (br s, 1H), 4.83 (br s, 1H), 4.76 (d, J=6.1 Hz, 2H), 4.30 (br s, 4H), 3.87-3.81 (m, 4H), 2.69 (dt, J=14.8, 5.1 Hz, 1H), 2.58 (ddd, J=14.6, 10.3, 4.9 Hz, 1H), 2.43-2.31 (m, 1H), 2.19 (s, 3H), 1.41 (br s, 1H), 1.16 (d, J=6.5 Hz, 3H). LCMS: (Method B) Rf=2.889 min, (ESI) m/z: 604.2, 606.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-morpholino-[1,2,4]triazolo[1,5-a]pyrazine-2-carboxamide [Cpd 072]

A mixture of (S)-N-(4-(1-acetyl-2-methyl-1,2,3,4-tetrahydroquinolin-6-yl)benzyl)-6-bromo-8-morpholino-[1,2,4]triazolo[1,5-a]pyrazine-2-carboxamide (S30.4) (100 mg, 0.17 mmol), 2-aminopyrimidine-5-boronic acid (35 mg, 0.25 mmol), K₂CO₃ (69 mg, 0.50 mmol) in dioxane/water (4:1, 10 mL) was degassed with N₂ for 15 min. PdCl₂(dppf) (12 mg, 0.02 mmol) was then added and the mixture was degassed for another 15 min. The reaction vessel was then evacuated and backfilled with N₂ three times and the reaction mixture was heated to reflux under N₂ o.n. The mixture was then diluted with 2% NaOH solution and extracted with DCM (×2). The organic extracts were combined, washed with brine, dried over MgSO₄ and concentrated in vacuo. The resulting residue was purified using flash column chromatography (0-6% MeOH in DCM) to give a light brown solid, which was triturated with MeOH and yielded the title compound as a white powder (65 mg, 64%). ¹H NMR (401 MHz, DMSO-d₆) δ 9.42 (br t, J=6.3 Hz, 1H), 8.91 (s, 2H), 8.84 (s, 1H), 7.63 (d, J=8.3 Hz, 2H), 7.53-7.35 (m, 5H), 6.99 (s, 2H), 4.69-4.59 (m, 1H), 4.55 (d, J=6.3 Hz, 2H), 4.25 (br s, 4H), 3.85-3.72 (m, 4H), 2.70 (dt, J=15.1, 5.3 Hz, 1H), 2.59-2.51 (m, 1H), 2.34-2.21 (m, 1H), 2.10 (s, 3H), 1.33 (br s, 1H), 1.05 (d, J=6.4 Hz, 3H). LCMS: (Method B) Rf=2.659 min, (ESI) m/z: 619.4 ([M+H]⁺, 100%). HRMS (ESI⁺) calcd for [C₃₃H₃₄N₁₀O₃+H]⁺619.2888, found 619.2906.

### 1,2-Diamino-5-bromo-3-morpholinopyridin-1-ium 2,4,6-trimethylbenzenesulfonate (S31.1)

Step 1: tert-butyl ((mesitylsulfonyl)oxy)carbamate (525 mg, 1.66 mmol) was added portionwise to ice-cold TFA (3 mL) and reaction mixture was then stirred at 0° C. for 1.5 hr. To this mixture was then added crushed ice and stirred vigorously at 0° C. Cold water was added to dilute the thick suspension and the white precipitate was filtered and washed with cold water. The wet solid was then re-dissolved with cold DCM, dried over MgSO₄ to prepare a DCM solution of O-(Mesitylenesulfonyl)hydroxylamine (MSH) (Note: dry MSH is potentially explo-sive, do not leave the MSH over-dry during filtration. Storage of the DCM solution is also not recommended even in the freezer). Step 2: To the cold DCM solution of MSH was added 5-bromo-3-morpholinopyridin-2-amine (S32.1) (330 mg, 1.28 mmol) and reaction mixture was stirred at 0° C. for 30 min before warming up to r.t o.n. The solvent was then concentrated in vacuo with gentle warming (water bath temperature˜20° C.). The crude title compound was obtained as a light brown amorphous solid and used directly in the next step without further purification. LCMS: (Method B) Rf=1.415 min, (ESI) m/z: 273.0, 275.0 ([M+H]⁺, 100%).

### Ethyl 6-bromo-8-morpholino-[1,2,4]triazolo[1,5-a]pyridine-2-carboxylate (S31.2)

The crude material (S31.1) from the last step was re-dissolved in pyridine (5 mL) and ethyl chlorooxoacetate (350 mg/285 uL, 2.56 mmol) was added dropwise. The reaction mixture was heated at 100° C. o.n. Pyridine was then removed in vacuo and sat. bicarb solution was added slowly to quench the residual acid at 0° C. for. The aqueous mixture was then extracted with DCM (×2), dried over MgSO₄ and concentrated in vacuo. The resulting crude material was then purified using flash column chromatography (0-15% EA in DCM) and obtained a dark yellow solid, which was triturated with cold diethyl ether to provide the title compound as a light yellow solid (335 mg, 74% over 2 steps). ¹H NMR (401 MHz, Chloroform-d) δ 8.34 (sd, J=1.6 Hz, 1H), 6.75 (sd, J=1.6 Hz, 1H), 4.53 (q, J=7.1 Hz, 2H), 3.97-3.91 (m, 4H), 3.69-3.64 (m, 4H), 1.47 (t, J=7.1 Hz, 3H). LCMS: (Method B) Rf=2.721 min, (ESI) m/z: 355.0, 357.0 ([M+H]⁺, 100%).

### 6-Bromo-8-morpholino-[1,2,4]triazolo[1,5-a]pyridine-2-carboxylic acid (S31.3)

A solution of ethyl 6-bromo-8-morpholino-[1,2,4]triazolo[1,5-a]pyridine-2-carboxylate (S31.2) (265 mg, 0.75 mmol) and LiOH·H₂O (94 mg, 2.24 mmol) in THE/water (3:1, 12 mL) was heated to reflux for 1 hr. THE was then removed in vacuo and the remaining aqueous residue was acidified to pH=1 with 1N HCl solution. The resulting precipitate was filtered and washed with water to give the title compound as a white solid (233 mg, 95%). ¹H NMR (401 MHz, DMSO-d₆) δ 8.87 (sd, J=1.5 Hz, 1H), 6.96 (sd, J=1.6 Hz, 1H), 3.80 (dd, J=6.1, 3.5 Hz, 4H), 3.61 (dd, J=5.9, 3.6 Hz, 4H). LCMS: (Method B) Rf=2.462 min, (ESI) m/z: 327.0, 329.0 ([M+H]⁺, 100%).

### (S)-N-(4-(1-Acetyl-2-methyl-1,2,3,4-tetrahydroquinolin-6-yl)benzyl)-6-bromo-8-morpholino-[1,2,4]triazolo[1,5-a]pyridine-2-carboxamide (S31.4)

A solution of (S)-1-(6-(4-(aminomethyl)phenyl)-2-methyl-3,4-dihydroquinolin-1(2H)-yl)ethan-1-one (S9.12) (90 mg, 0.31 mmol), 6-bromo-8-morpholino-[1,2,4]triazolo[1,5-a]pyri-dine-2-carboxylic acid (S31.3) (120 mg, 0.37 mmol), PyClock (340 mg, 0.61 mmol) and DIPEA (238 mg/320 uL, 1.84 mmol) in DMF (10 mL) was stirred at r.t o.n. The reaction was then quenched by adding into 5% Na₂CO₃ solution. The resulting precipitate was filtered and purified using flash column chromatography (0-75% EA in DCM) to give the title compound as a beige powder (180 mg, 98%). ¹H NMR (401 MHz, Chloroform-d) δ 8.35 (sd, J=1.6 Hz, 1H), 7.62 (br t, J=6.2 Hz, 1H), 7.57 (d with fine splitting, J=8.3 Hz, 2H), 7.45 (d with fine splitting, J=8.2 Hz, 2H), 7.41 (dd, J=8.2, 1.9 Hz, 1H), 7.37 (sd, J=2.0 Hz, 1H), 7.20 (br s, 1H), 6.77 (sd, J=1.5 Hz, 1H), 4.83 (br s, 1H), 4.77 (d, J=6.2 Hz, 2H), 3.96-3.89 (m, 4H), 3.66-3.54 (m, 4H), 2.69 (dt, J=14.8, 5.0 Hz, 1H), 2.58 (ddd, J=14.9, 10.8, 5.0 Hz, 1H), 2.43-2.31 (m, 1H), 2.19 (s, 3H), 1.41 (br s, 1H), 1.16 (d, J=6.5 Hz, 3H). LCMS: (Method B) Rf=2.822 min, (ESI) m/z: 603.2, 605.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-morpholino-[1,2,4]triazolo[1,5-a]pyridine-2-carboxamide [Cpd 073]

A mixture of (S)-N-(4-(1-acetyl-2-methyl-1,2,3,4-tetrahydroquinolin-6-yl)benzyl)-6-bromo-8-morpholino-[1,2,4]triazolo[1,5-a]pyridine-2-carboxamide (S31.4) (120 mg, 0.20 mmol), 2-aminopyrimidine-5-boronic acid (56 mg, 0.40 mmol), K₂CO₃ (110 mg, 0.80 mmol) in dioxane/water (4:1, 10 mL) was degassed with N₂ for 10 min. PdCl₂(PPh₃)₂ (15 mg, 0.02 mmol) was then added and the mixture was degassed for another 10 min. The reaction vessel was then evacuated and backfilled with N₂ three times and the reaction mixture was heated to reflux under N₂ o.n. The mixture was then diluted with 2% NaOH solution and extracted with DCM (×2). The organic extracts were combined, washed with brine, dried over MgSO₄ and concentrated in vacuo. The residue was then purified using flash column chromatography (0-6% MeOH in DCM) and obtained a light brown solid, which was triturated with cold MeOH to provide the title compound as a white powder (98 mg, 80%). ¹H NMR (401 MHz, DMSO-d₆) δ 9.35 (br t, J=6.3 Hz, 1H), 8.82 (sd, J=1.4 Hz, 1H), 8.71 (s, 2H), 7.64 (d, J=8.3 Hz, 2H), 7.53-7.35 (m, 5H), 7.09 (d, J=1.5 Hz, 1H), 6.93 (s, 2H), 4.70-4.59 (m, 1H), 4.56 (d, J=6.3 Hz, 2H), 3.91-3.76 (m, 4H), 3.73-3.59 (m, 4H), 2.70 (dt, J=14.9, 5.2 Hz, 1H), 2.60-2.51 (m, 1H), 2.37-2.21 (m, 1H), 2.10 (s, 3H), 1.33 (br s, 1H), 1.05 (d, J=6.4 Hz, 3H). LCMS: (Method B) Rf=2.618 min, (ESI) m/z: 618.4 ([M+H]⁺, 100%). HRMS (ESI⁺) calcd for [C₃₄H₃₅N₉O₃+H]⁺618.2936, found 618.2951.

### 5-Bromo-3-morpholinopyridin-2-amine (S32.1)

The reaction vessel was charged with 3,5-dibromopyridin-2-amine (1.01 g, 4.01 mmol) and XPhos Pd G1 (0.12 g, 0.16 mmol) then evacuated and backfilled with N₂ (×3). Morpholine (0.53 g/0.52 mL, 6.03 mmol) was then added followed by LiHMDS (1M in THF, 10 mL, 10 mmol). The reaction mixture was heated at 65° C. o.n then quenched by adding sat. NH₄C₁ solution (10 mL). The mixture was further diluted with EA (100 mL) and passed through layer of celite to remove any insoluble material. The filtrate was then washed with sat. bicarb solution (×2), dried over MgSO₄ and concentrated in vacuo. The crude residue was purified using flash column chromatography (0-30% EA in PE). Fraction containing the desired product were concentrated and triturated with PE to give the title compound as a beige powder (340 mg, 33%). ¹H NMR (401 MHz, DMSO-d₆) δ 7.74 (sd, J=2.2 Hz, 1H), 7.25 (sd, J=2.2 Hz, 1H), 5.86 (s, 2H), 3.80-3.69 (m, 4H), 2.86-2.74 (m, 4H). LCMS: (Method B) Rf=2.370 min, (ESI) m/z: 258.0, 260.0 ([M+H]⁺, 100%).

### Ethyl 6-bromo-8-morpholinoimidazo[1,2-a]pyridine-2-carboxylate (S32.2)

To a sealed vial was added 5-bromo-3-morpholinopyridin-2-amine (S32.1) (310 mg, 1.20 mmol), ethyl bromopyruvate (352 mg/226 uL, 1.80 mmol) and dry dioxane (3 mL). The reaction mixture was then heated at 100° C. o.n. After cooling to r.t, the reaction mixture was diluted with DCM, washed with sat. bicarb solution (×2), dried over MgSO₄ and concentrated in vacuo. The crude residue was then purified using flash column chromatography (0-30% EA in DCM). Fractions containing the desired product were concentrated and triturated with cold ether to give the title compound as a light yellow powder (65 mg, 15%). ¹H NMR (400 MHz, Chloroform-d) δ 8.03 (s, 1H), 7.87 (sd, J=1.6 Hz, 1H), 6.45 (sd, J=1.6 Hz, 1H), 4.42 (q, J=7.1 Hz, 2H), 4.01-3.92 (m, 4H), 3.69-3.59 (m, 4H), 1.41 (t, J=7.1 Hz, 3H). LCMS: (Method B) Rf=2.781 min, (ESI) m/z: 354.1, 356.1 ([M+H]⁺, 100%).

### 6-Bromo-8-morpholinoimidazo[1,2-a]pyridine-2-carboxylic acid (S32.3)

A solution of ethyl 6-bromo-8-morpholinoimidazo[1,2-a]pyridine-2-carboxylate (S32.2) (60 mg, 0.17 mmol) and LiOH·H₂O (72 mg, 1.71 mmol) in THF/water (3:1, 8 mL) was heated to reflux for 1.5 hr. THF was then removed in vacuo and the remaining aqueous residue was acidified to pH=1 with 1N HCl solution. The aqueous solution was then freeze-dried to obtain the crude title compound which was contaminated with excess amount of LiCl. This was then used in the next step without further purification. LCMS: (Method B) Rf=2.516 min, (ESI) m/z: 326.0, 328.0 ([M+H]⁺, 100%).

### (S)-N-(4-(1-Acetyl-2-methyl-1,2,3,4-tetrahydroquinolin-6-yl)benzyl)-6-bromo-8-morpholinoimidazo[1,2-a]pyridine-2-carboxamide (S32.4)

A solution of the crude acid starting material (S32.3), (S)-1-(6-(4-(aminomethyl)phenyl)-2-methyl-3,4-dihydroquinolin-1(2H)-yl)ethan-1-one (59.12) (60 mg, 0.20 mmol), PyClock (205 mg, 0.37 mmol) and DIPEA (143 mg/193 uL, 1.11 mmol) in DMF (8 mL) was stirred at r.t o.n. Another portion of the amine (17 mg, 0.06 mmol), PyClock (102 mg, 0.18 mmol) and DIPEA (72 mg/97 uL, 0.56 mmol) were added and reaction mixture was stirred for another 8 hr. The reaction was then quenched by adding into 5% Na₂CO₃ solution. The resulting precipitate was filtered and purified using flash column chromatography (0-70% EA in DCM) to give an oily residue, which was co-evaporated in DCM/diethyl ether to provide the title compound as an off-white solid (60 mg, 59% over 2 steps). ¹H NMR (401 MHz, Chloroform-d) δ 8.08 (s, 1H), 7.95 (d, J=1.6 Hz, 1H), 7.73 (d, J=12.7 Hz, 1H), 7.56 (d, J=8.3 Hz, 2H), 7.44 (d, J=8.2 Hz, 3H), 7.41 (dd, J=8.2, 2.1 Hz, 3H), 7.37 (s, 1H), 7.20 (br s, 1H), 6.54 (s, 1H), 4.84 (br s, 1H), 4.72 (d, J=6.2 Hz, 2H), 4.01-3.87 (m, 4H), 3.59-3.50 (m, 4H), 2.76-2.63 (m, 1H), 2.64-2.51 (m, 1H), 2.44-2.30 (m, 1H), 2.19 (s, 3H), 1.39 (br s, 1H), 1.16 (d, J=6.4 Hz, 4H). LCMS: (Method B) Rf=2.822 min, (ESI) m/z: 602.2, 604.2 ([M+H]⁺, 100%).

### (S)-N-(4-(1-Acetyl-2-methyl-1,2,3,4-tetrahydroquinolin-6-yl)benzyl)-6-(2-aminopyrim-idin-5-yl)-8-morpholinoimidazo[1,2-a]pyridine-2-carboxamide [Cpd 074]

A mixture of (S)-N-(4-(1-acetyl-2-methyl-1,2,3,4-tetrahydroquinolin-6-yl)benzyl)-6-bromo-8-morpholinoimidazo[1,2-a]pyridine-2-carboxamide (S32.4) (50 mg, 0.08 mmol), 2-aminopyrimidine-5-boronic acid (18 mg, 0.13 mmol), K₂CO₃ (46 mg, 0.33 mmol) in dioxane/water (4:1, 10 mL) was degassed with N₂ for 10 min. PdCl₂(PPh₃)₂ (12 mg, 0.02 mmol) was then added and the mixture was degassed for another 10 min. The reaction vessel was then evacuated and backfilled with N₂ three times and the reaction mixture was heated to reflux under N₂ o.n. The mixture was then diluted with 2% NaOH solution and extracted with DCM (×2). The organic extracts were combined, washed with brine, dried over MgSO₄ and concentrated in vacuo. The resulting residue was purified using preparative TLC (5% MeOH in DCM) to give the title compound as an off-white powder (12 mg, 23%). ¹H NMR (401 MHz, DMSO-d₆) δ 8.87 (br t, J=6.4 Hz, 1H), 8.61 (s, 2H), 8.45 (sd, J=1.4 Hz, 1H), 8.27 (s, 1H), 7.64 (d, J=8.2 Hz, 2H), 7.53-7.45 (m, 2H), 7.41 (app. d, 3H), 6.85 (s, 2H), 6.72 (sd, J=1.5 Hz, 1H), 4.71-4.60 (m, 1H), 4.55 (d, J=6.4 Hz, 2H), 3.91-3.77 (m, 4H), 3.72-3.56 (m, 4H), 2.76-2.64 (m, 1H), 2.60-2.52 (m, 1H), 2.35-2.22 (m, 1H), 2.11 (s, 3H), 1.34 (br s, 1H), 1.06 (d, J=6.5 Hz, 3H). LCMS: (Method B) Rf=2.617 min, (ESI) m/z: 617.4 ([M+H]⁺, 100%). HRMS (ESI⁺) calcd for [C₃₅H₃₆N₈O₃+H]⁺617.2983, found 617.2995.

### Ethyl 5,7-dioxo-4,5,6,7-tetrahydropyrazolo[1,5-a]pyrimidine-2-carboxylate (S33.1)

A suspension of ethyl 5-amino-1H-pyrazole-3-carboxylate (2.00 g, 12.89 mmol) and diethyl malonate (4.20 g/4.00 mL, 26.22 mmol) in abs. EtOH (50 mL) was degassed with N₂ for 15 min. Sodium ethoxide (1.95 g, 28.66 mmol) was added under N₂ atmosphere. The reaction vessel was then evacuated and backfilled with N₂ (×3) and the reaction mixture was heated to reflux o.n. The solid material was then collected via filtration, washed with EtOH and used in the next step without further purification.

### Ethyl 5,7-dichloropyrazolo[1,5-a]pyrimidine-2-carboxylate (S33.2)

The solid material (S33.1) from the last step was suspended in POCl₃ (18 mL) and heated at 100° C. o.n. The reaction mixture was then quenched by slowly adding into an ice-water mixture with vigorous stirring. Upon complete quenching of POCl₃, the mixture was filtered to removed any insoluble material. The filtrate was then extracted with DCM (×2). The organic extracts were combined, washed with brine, dried over MgSO₄ and concentrated in vacuo. The crude material was then used directly in the next step without further purification.

¹H NMR (401 MHz, Chloroform-d) δ 7.22 (s, 1H), 7.11 (s, 1H), 4.50 (q, J=7.1 Hz, 2H), 1.45 (t, J=7.1 Hz, 3H). LCMS: (Method B) Rf=2.760 min, (ESI) m/z: 259.1 ([M+H]⁺, 100%).

### Ethyl 5-chloro-7-morpholinopyrazolo[1,5-a]pyrimidine-2-carboxylate (S33.3)

The crude material (S33.2) was re-dissolved in DCM (30 mL) and morpholine was added dropwise. The reaction mixture was then stirred at r.t for 1 hr. More DCM was added to dilute the reaction mixture and then washed with water (×1), brine (×1), dried over MgSO₄ and concentrated in vacuo. The crude residue was then purified using flash column chromatography (0-25% EA in DCM) to give the title compound as a white solid (80 mg, 2% over 3 steps).

¹H NMR (401 MHz, Chloroform-d) δ 6.98 (s, 1H), 6.17 (s, 1H), 4.45 (q, J=7.1 Hz, 2H), 4.03-3.93 (m, 4H), 3.89-3.80 (m, 4H), 1.43 (t, J=7.1 Hz, 3H). LCMS: (Method B) Rf=2.760 min, (ESI) m/z: 311.1 ([M+H]⁺, 100%).

### 5-Chloro-7-morpholinopyrazolo[1,5-a]pyrimidine-2-carboxylic acid (S33.4)

To a solution of ethyl 5-chloro-7-morpholinopyrazolo[1,5-a]pyrimidine-2-carboxylate (S33.3) (75 mg, 0.24 mmol) in THE/water (4:1, 10 mL) was added LiOH·H₂O (51 mg, 1.21 mmol). The reaction mixture was then heated at 50° C. for 1.5 hr. THE was removed in vacuo and the remaining aqueous residue was acidified to pH=1 with 1N HCl solution. The resulting precipitate was filtered and washed with water to give the title compound as a white solid (25 mg, 37%). ¹H NMR (401 MHz, DMSO-d₆) δ 6.82 (s, 1H), 6.55 (s, 1H), 3.70-3.61 (m, 4H), 3.54-3.48 (m, 4H). LCMS: (Method B) Rf=2.497 min, (ESI) m/z: 283.0 ([M+H]⁺, 100%).

### (S)-N-(4-(1-Acetyl-2-methyl-1,2,3,4-tetrahydroquinolin-6-yl)benzyl)-5-chloro-7-morpholinopyrazolo[1,5-a]pyrimidine-2-carboxamide (S33.5)

A solution of (S)-1-(6-(4-(aminomethyl)phenyl)-2-methyl-3,4-dihydroquinolin-1(2H)-yl)ethan-1-one (59.12) (23 mg, 0.08 mmol), 5-chloro-7-morpholinopyrazolo[1,5-a]pyrimidine-2-carboxylic acid (S33.4) (20 mg, 0.07 mmol), PyClock (79 mg, 0.14 mmol), DIPEA (55 mg/75 uL, 0.43 mmol) in DMF (5 mL) was stirred at r.t o.n. The reaction was then quenched by adding into 5% Na₂CO₃ solution, the resulting precipitate was filtered, washed with water and used directly in the next step without further purification. LCMS: (Method B) Rf=2.783 min, (ESI) m/z: 559.2 ([M+H]⁺, 100%).

### (S)-N-(4-(1-Acetyl-2-methyl-1,2,3,4-tetrahydroquinolin-6-yl)benzyl)-5-(2-aminopyrim-idin-5-yl)-7-morpholinopyrazolo[1,5-a]pyrimidine-2-carboxamide [Cpd 075]

A mixture of (S)-N-(4-(1-acetyl-2-methyl-1,2,3,4-tetrahydroquinolin-6-yl)benzyl)-6-bromo-8-morpholino-[1,2,4]triazolo[1,5-a]pyrazine-2-carboxamide (S33.5) (45 mg crude material from the last step), 2-aminopyrimidine-5-boronic acid (22 mg, 0.16 mmol), K₂CO₃ (45 mg, 0.33 mmol) in dioxane/water (4:1, 10 mL) was degassed with N₂ for 10 min. PdCl₂(PPh₃)₂ (12 mg, 0.02 mmol) was then added and the mixture was degassed for another 10 min. The reaction vessel was then evacuated and backfilled with N₂ three times and the reaction mixture was heated to reflux under N₂ o.n. The mixture was then diluted with 2% NaOH solution and extracted with DCM (×2). The organic extracts were combined, washed with brine, dried over MgSO₄ and concentrated in vacuo. The resulting residue was purified using flash column chromatography (0-6% MeOH in DCM) to give a light yellow solid, which was triturated with MeOH and yielded the title compound as an off-white powder (27 mg, 62% over 2 steps). ¹H NMR (401 MHz, DMSO-d₆) δ 9.11-9.01 (m, 3H), 7.65 (d, J=8.3 Hz, 2H), 7.53-7.45 (m, 2H), 7.42 (app. d, 3H), 7.20 (s, 2H), 6.91 (s, 1H), 6.86 (s, 1H), 4.70-4.60 (m, 1H), 4.57 (d, J=6.3 Hz, 2H), 3.95-3.78 (m, 8H), 2.77-2.63 (m, 1H), 2.60-2.52 (m, 1H), 2.35-2.21 (m, 1H), 2.11 (s, 3H), 1.34 (br s, 1H), 1.06 (d, J=6.4 Hz, 3H). LCMS: (Method B) Rf=2.623 min, (ESI) m/z: 618.3 ([M+H]⁺, 100%). HRMS (ESI⁺) calcd for [C₃₄H₃₅N₉O₃+H]⁺618.2936, found 618.2953.

### 1-(5-(4-(Aminomethyl)phenyl)-2-methylindolin-1-yl)ethan-1-one (534.1)

A solution of 1-(5-bromo-2-methylindolin-1-yl)ethan-1-one (300 mg, 1.18 mmol), 4-aminomethylphenylboronic acid, hydrochloride (266 mg, 1.42 mmol), K₂CO₃ (653 mg, 4.72 mmol) in a mixture of DME/H₂O (4:1, 10 mL) was degassed with N₂ for 15 min. Pd(dppf)Cl₂ (87 mg, 0.12 mmol) was then added and the mixture was further degassed for another 15 min. The reaction vessel was then evacuated and backfilled with N₂ three times. The reaction mixture was heated to reflux under N₂ for 4 hr. After cooling to r.t, the reaction mixture was then diluted with 1N HCl solution and washed with DCM (×2). The aqueous layer was adjusted to pH=14 with 10% NaOH solution and extracted with DCM (×2). The organic extracts were combined, dried over MgSO₄ and concentrated in vacuo to give the title compound as a light-yellow solid (260 mg, 79%). ¹H NMR (401 MHz, DMSO-d₆) δ 8.04 (d, J=8.4 Hz, 1H), 7.60-7.51 (m, 3H), 7.45 (br d, J=8.2 Hz, 1H), 7.38 (d, J=7.9 Hz, 2H), 4.69-4.56 (m, 1H), 3.73 (s, 2H), 3.47-3.41 (m, 1H), 2.71 (br d, J=16.0 Hz, 1H), 2.23 (s, 3H), 1.24 (d, J=6.4 Hz, 3H). LCMS: (Method B) Rf=2.283 min, (ESI) m/z: 264.1 ([M−NH₂]⁺, 100%), 281.1 ([M+H]⁺, 10%).

### 1-(5-(4-(Aminomethyl)phenyl)-2-methyl-4-propoxyindolin-1-yl)ethan-1-one (S34.3)

A solution of 1-(5-bromo-2-methyl-4-propoxyindolin-1-yl)ethan-1-one (80 mg, 0.26 mmol), 4-aminomethylphenylboronic acid pinacol ester, hydrochloride (58 mg, 0.31 mmol), K₂CO₃ (142 mg, 1.03 mmol) in a mixture of DME/H₂O (4:1, 5 mL) was degassed with N₂ for 15 min. Pd(dppf)Cl₂ (19 mg, 0.03 mmol) was then added and the mixture was further degassed for another 15 min. The reaction vessel was then evacuated and backfilled with N₂ three times. The reaction mixture was heated to reflux under N₂ for 4 hr. After cooling to r.t, the reaction mixture was then diluted with 1N HCl solution and washed with DCM (×2). The aqueous layer was adjusted to pH=14 with 10% NaOH solution and extracted with DCM (×2). The organic extracts were combined, dried over MgSO₄ and concentrated in vacuo to give the title compound as a light-yellow solid (38 mg, 44%). LCMS: (Method B) Rf=2.381 min, (ESI) m/z: 322.2 ([M−NH₂]⁺, 100%), 339.2 ([M+H]⁺, 10%).

### N-(4-(1-Acetyl-2-methylindolin-5-yl)benzyl)-6-bromo-8-morpholinoimidazo[1,2-a]pyrazine-2-carboxamide (S34.2)

A solution of 1-(5-(4-(aminomethyl)phenyl)-2-methylindolin-1-yl)ethan-1-one (S34.1) (100 mg, 0.36 mmol), lithium(I) 6-bromo-8-morpholinoimidazo[1,2-a]pyrazine-2-carboxylate (S1.3) (155 mg, 0.47 mmol), PyClock (396 mg, 0.71 mmol), DIPEA (277 mg/373 μL, 2.14 mmol) in DMF (5 mL) was stirred at r.t o.n. The reaction mixture was then poured into 5% Na₂CO₃ solution, the resulting precipitate was filtered and purified using flash column chromatography (0-2% MeOH in DCM) to yield a clear oil which crystallised on standing to give the title compound as a white solid (171 mg, 81%). ¹H NMR (401 MHz, DMSO-d₆) δ 9.06 (br t, J=6.4 Hz, 1H), 8.31 (s, 1H), 8.17 (s, 1H), 8.04 (br d, J=8.4 Hz, 1H), 7.60 (d, J=8.0 Hz, 2H), 7.55 (br s, 1H), 7.46 (br d, J=8.5 Hz, 1H), 7.38 (d, J=8.0 Hz, 2H), 4.69-4.59 (m, 1H), 4.53 (d, J=6.3 Hz, 2H), 4.26 (br t, 4H), 3.76 (app. t, 4H), 3.48-3.40 (m, 1H), 2.77-2.65 (m, 1H), 2.24 (s, 3H), 1.24 (d, J=6.2 Hz, 3H). LCMS: (Method B) Rf=3.017 min, (ESI) m/z: 589.2, 591.2 ([M+H]⁺, 100%).

### N-(4-(1-Acetyl-2-methyl-4-propoxyindolin-5-yl)benzyl)-6-bromo-8-morpholinoimidazo[1,2-a]pyrazine-2-carboxamide (S34.4)

A solution of 1-(5-(4-(aminomethyl)phenyl)-2-methyl-4-propoxyindolin-1-yl)ethan-1-one (S34.3) (25 mg, 0.07 mmol), lithium(I) 6-bromo-8-morpholinoimidazo[1,2-a]pyrazine-2-carboxylate (S1.3) (32 mg, 0.10 mmol), PyClock (82 mg, 0.15 mmol), DIPEA (58 mg/78 μL, 2.14 mmol) in DMF (3 mL) was stirred at r.t o.n. The reaction mixture was then poured into 5% Na₂CO₃ solution, the resulting precipitate was filtered and purified using flash column chromatography (0-2% MeOH in DCM) to yield a clear oil which crystallised on standing to give the title compound as an off-white solid (40 mg, 83%). LCMS: (Method B) Rf=3.124 min, (ESI) m/z: 647.2, 649.2 ([M+H]⁺, 100%).

### N-(4-(1-Acetyl-2-methylindolin-5-yl)benzyl)-6-(2-aminopyrimidin-5-yl)-8-morpholinoimidazo[1,2-a]pyrazine-2-carboxamide [Cpd 076]

A solution of N-(4-(1-acetyl-2-methylindolin-5-yl)benzyl)-6-bromo-8-morpholinoimidazo[1,2-a]pyrazine-2-carboxamide (S34.2) (100 mg, 0.17 mmol), 2-aminopyrimidine-5-boronic acid (29 mg, 0.21 mmol), K₂CO₃ (71 mg, 0.51 mmol) in a mixture of DME/H₂O (4:1, 10 mL) was degassed with N₂ for 15 min. Pd(dppf)Cl₂ (13 mg, 0.02 mmol) was then added and the mixture was further degassed for another 15 min. The reaction vessel was then evacuated and backfilled with N₂ three times. The reaction mixture was heated to reflux under N₂ for 4 hr. The mixture was then diluted with water and extracted with a mixture of DCM/isopropanol (3:1, ×3). The organic extracts were combined, dried over MgSO₄ and concentrated in vacuo. The resulting residue was purified using flash column chromatography (0-5% MeOH in DCM) to give a light yellow solid, which was triturated with MeOH to give the title compound as a white powder (73 mg, 71%). ¹H NMR (401 MHz, DMSO-d₆) δ 9.02 (br t, J=6.4 Hz, 1H), 8.78 (s, 2H), 8.46 (s, 1H), 8.28 (s, 1H), 8.04 (br d, J=8.5 Hz, 1H), 7.60 (d, J=8.1 Hz, 2H), 7.54 (br s, 1H), 7.45 (br d, J=8.5 Hz, 1H), 7.38 (d, J=8.1 Hz, 2H), 6.89 (s, 2H), 4.70-4.57 (m, 1H), 4.53 (d, J=6.4 Hz, 2H), 4.35-4.23 (m, 4H), 3.79 (app. t, 4H), 3.47-3.40 (m, 1H), 2.76-2.64 (m, 1H), 2.23 (s, 3H), 1.24 (d, J=6.2 Hz, 3H). LCMS: (Method B) Rf=2.735 min, (ESI) m/z: 604.3 ([M+H]⁺, 100%). HRMS (ESI⁺) calcd for [C₃₃H₃₃N₉O₃+H]⁺604.2779, found 604.2807.

### N-(4-(1-Acetyl-2-methyl-4-propoxyindolin-5-yl)benzyl)-6-(2-aminopyrimidin-5-yl)-8-morpholinoimidazo[1,2-a]pyrazine-2-carboxamide [Cpd 077]

A solution of N-(4-(1-acetyl-2-methyl-4-propoxyindolin-5-yl)benzyl)-6-bromo-8-morpholinoimidazo[1,2-a]pyrazine-2-carboxamide (S34.4) (40 mg, 0.06 mmol), 2-aminopyrimi-dine-5-boronic acid (12 mg, 0.09 mmol), K₂CO₃ (26 mg, 0.19 mmol) in a mixture of DME/H₂O (4:1, 5 mL) was degassed with N₂ for 10 min. Pd(dppf)Cl₂ (5 mg, 0.01 mmol) was then added and the mixture was further degassed for another 10 min. The reaction vessel was then evacuated and backfilled with N₂ three times. The reaction mixture was heated to reflux under N₂ for 4 hr. The mixture was then diluted with water and extracted with DCM (×2). The organic extracts were combined, dried over MgSO₄ and concentrated in vacuo. The resulting residue was purified using flash column chromatography (0-5% MeOH in DCM) to give a light brown solid, which was triturated with MeOH to give the title compound as an off-white powder (26 mg, 63%). ¹H NMR (401 MHz, DMSO-d₆) δ 9.03 (br t, J=6.5 Hz, 1H), 8.78 (s, 2H), 8.46 (s, 1H), 8.28 (s, 1H), 7.79 (br d, J=8.1 Hz, 1H), 7.48 (d, J=8.2 Hz, 2H), 7.36 (d, J=8.1 Hz, 2H), 7.15 (br d, J=8.2 Hz, 1H), 6.89 (s, 2H), 4.76-4.59 (m, 1H), 4.55 (d, J=6.4 Hz, 2H), 4.38-4.21 (m, 4H), 3.79 (app. t, 4H), 3.62-3.46 (m, 2H), 3.44-3.37 (m, 1H), 2.79-2.62 (m, 1H), 2.22 (s, 3H), 1.46 (h, J=7.1 Hz, 2H), 1.31-1.13 (m, 3H), 0.79 (t, J=7.4 Hz, 3H). ˜10% of rotamer exists. LCMS: (Method B) Rf=2.802 min, (ESI) m/z: 662.3 ([M+H]⁺, 100%). HRMS (ESI⁺) calcd for [C₃₆H₃₉N₉O₄+H]⁺662.3198, found 662.3222.

### (S)-1-(6-(4-((((6-bromo-8-morpholinoimidazo[1,2-a]pyrazin-2-yl)methyl)(hydroxy)amino)methyl)phenyl)-2-methyl-3,4-dihydroquinolin-1(2H)-yl)ethan-1-one (S20.1)

A solution of (S)-1-(6-(4-((hydroxyamino)methyl)phenyl)-2-methyl-3,4-dihydroquino-lin-1(2H)-yl)ethan-1-one (510.4) (1.2 eq.), 4-(6-bromo-2-(bromomethyl)imidazo[1,2-a]pyra-zin-8-yl)morpholine (S13.7) (1 eq.) and TEA (2 eq.) in THE will be heated to reflux o.n. The reaction mixture will then be concentrated and purified using flash column chromatography to afford the title compound.

### (S)-1-(6-(4-((((6-(2-Aminopyrimidin-5-yl)-8-morpholinoimidazo[1,2-a]pyrazin-2-yl)methyl)(hydroxy)amino)methyl)phenyl)-2-methyl-3,4-dihydroquinolin-1(2H)-yl)ethan-1-one [Cpd 078]

A solution of (S)-1-(6-(4-((((6-bromo-8-morpholinoimidazo[1,2-a]pyrazin-2-yl)methyl)(hydroxy)amino)methyl)phenyl)-2-methyl-3,4-dihydroquinolin-1(2H)-yl)ethan-1-one (S20.1) (1 eq.), 2-amino-4-(trifluoromethyl)pyridine-5-boronic acid pinacol ester (1.2 eq.), K₂CO₃ (3 eq.), in a mixture of DME/H₂O (4:1) will be degassed with N₂ for 15 min. Pd(dppf)Cl₂ (0.1 eq.) will then be added and the mixture further degassed for another 15 min. The reaction vessel will then be evacuated and backfilled with N₂ three times. The reaction mixture will be heated to reflux under N₂ for 4 hr. The mixture will then be diluted with water and extracted with DCM (×3). The organic extracts will be combined, dried over MgSO₄ and concentrated in vacuo. The resulting residue will be purified using flash column chromatography to afford the title compound.

### Biological Evaluation of Dual Inhibitor Compounds

### Cell Proliferation Assays

OPM2 cells (1×10³) were plated in a 96 well plate. Test compounds were added to cells at final concentrations ranging from 5 μM to 1 nM. They were incubated for 5 days before they were analysed using ViaLight luminescence-based proliferation assays which detect cellular ATP, as per the manufacturer's instructions. All samples were prepared in technical triplicates. Data analysis was performed in GraphPad Prism version 7.00 for Mac OS X, (GraphPad Software, San Diego California USA) in order to calculate the GI₅₀.

The results of the proliferation assay are shown in Table 1.

### PI3K Enzyme Activity Assay

PI3K activity was determined using a luminescence assay measuring ATP consumption as described previously (J.-A. Pinson, Z. Zheng, M. S. Miller, D. K. Chalmers, I. G. Jennings, P. E. Thompson, L-Aminoacyl-triazine derivatives are isoform-selective PI3Kβ inhibitors that target nonconserved Asp862 of PI3Kb, ACS Med. Chem. Lett. 4 (2013) 206-210). PI3K enzyme activity was determined in 25 μl of 20 mM HEPES pH 7.5, 5 mM MgCl₂ with 180 μM phosphatidyl inositol and 10 μMATP. After a 60 min incubation at room temperature the reaction was stopped by the addition of Kinase-Glo (Promega) followed by a further 15 min incubation. Luminescence was then read using a Fluostar plate reader (BMGLabtech).

Inhibitors were diluted in 20% (v/v) DMSO at the indicated concentrations in order to generate a concentration versus inhibition of enzyme activity curve which was then analysed using GraphPad Prism version 8.00 for Windows, (GraphPad Software, San Diego California USA) in order to calculate the IC₅₀. Standard deviations for the calculated IC₅₀'s are within 25% of the mean.

The results of the PI3K enzyme activity assay are shown in Table 1.

### Bromodomain Binding Assay

Inhibition of bromodomain binding was measured using a FRET (fluorescence resonance energy transfer) based assay. GST-BRD4 (binding domain 1, amino acids 44-168) was incubated with the tetracetylated histone 4 peptide (amino acid sequence SGRGK(ac)GGK(ac)GLGK(ac)GGAK(ac)RHRKV) which had a C-terminal biotin. In the presence of the two detection reagents, streptavidin D2 and anti-GST-Eu (CisBio) binding trig-gers a FRET signal detected by fluorescence at 665 nm as measured using a Pherastar reader (BMG Labtech). Data was analysed using GraphPad Prism version 8.00 for Windows, (GraphPad Software, San Diego California USA) in order to calculate the IC₅₀. Standard deviations for the calculated IC₅₀'s are within 25% of the mean.

The results of the bromodomain binding assay are shown in Table 1.

TABLE 1
Results of the cell proliferation assay, PI3K enzyme
activity assay, and bromodomain binding assay.

| | Cell | PI3K Enzyme | Bromodomain |
|---|---|---|---|
| | Proliferation | Activity | Binding |
| | Assay | Assay | Assay (BRD4) |
| Cpd | GI₅₀ (nM) | IC₅₀ (nM) | IC₅₀ (nM) |
| 001 | ++ | + | + |
| 002 | +++ | +++ | ++ |
| 003 | ++ | +++ | + |
| 004 | ND | ++ | ++ |
| 005 | +++ | +++ | ++ |
| 006 | ++ | +++ | ++ |
| 007 | ND | +++ | + |
| 008 | ND | +++ | + |
| 009 | +++ | ++ | +++ |
| 010 | +++ | ND | ND |
| 011 | ++ | ND | ND |
| 012 | +++ | ND | ND |
| 013 | + | +++ | +++ |
| 014 | ++ | ND | ND |
| 015 | ++ | ND | ND |
| 016 | ++ | ND | ND |
| 017 | ++ | ND | ND |
| 018 | ++ | ND | ND |
| 019 | ++ | ND | ND |
| 020 | +++ | +++ | ND |
| 021 | +++ | +++ | ND |
| 022 | +++ | ND | ND |
| 023 | ++ | ND | ND |
| 024 | + | ND | ND |
| 025 | ++ | ND | ND |
| 026 | +++ | ++ | ND |
| 027 | ++ | +++ | ND |
| 028 | ++ | ++ | ND |
| 029 | ++ | ++ | ND |
| 030 | +++ | + | ND |
| 031 | +++ | ++ | ND |
| 032 | +++ | + | ND |
| 033 | +++ | +++ | ND |
| 035 | +++ | ND | ND |
| 036 | + | ND | ND |
| 037 | ++ | ND | ND |
| 038 | + | ND | ND |
| 039 | + | ND | ND |
| 040 | + | ND | ND |
| 041 | + | ND | ND |
| 042 | + | ND | ND |
| 043 | + | ND | ND |
| 044 | ++ | ND | ND |
| 045 | +++ | ND | ND |
| 046 | ++ | ND | ND |
| 047 | + | ND | ND |
| 048 | +++ | ND | ND |
| 049 | ++ | ND | ND |
| 050 | ++ | ND | ND |
| 051 | ++ | ND | ND |
| 052 | + | ND | ND |
| 053 | +++ | ND | ND |
| 055 | + | ND | ND |
| 056 | ++ | ND | ND |
| 057 | + | ND | ND |
| 058 | ++ | ND | ND |
| 059 | +++ | ND | ND |
| 060 | +++ | ND | ND |
| 061 | ++ | ND | ND |
| 062 | +++ | ND | ND |
| 063 | +++ | ND | ND |
| 064 | ++ | ND | ND |
| 065 | +++ | ND | ND |
| 066 | +++ | ND | ND |
| 067 | +++ | ND | ND |
| 068 | +++ | ND | ND |
| 069 | +++ | ND | ND |
| 070 | +++ | ND | ND |
| 071 | +++ | ND | ND |
| 072 | +++ | ND | ND |
| 073 | +++ | ND | ND |
| 074 | +++ | ND | ND |
| 075 | +++ | ND | ND |

+++ denotes ≤100 nM.
++ denotes 100 nM-500 nM.
+ denotes >500 nM.
ND: Not determined.

### Apoptosis Assay

A panel of human multiple myeloma cell lines (3×10⁵) was incubated in the presence of Cpd 010 or DMSO vehicle control with indicated concentrations for 48 h before flow cytometric analysis of viability using Tetramethylrhodamine ethylester (TMRE) stains. Further apoptotic assay using only OPM2 cells was carried out using Cpd 010 or DMSO with TMRE stains.

The results, shown in FIG. 1, demonstrate that Cpd 010 induced dose-dependent apoptosis in five of the six treated cells. OPM2 cells were the most sensitive and U266 was the least sensitive to Cpd 010 (Note: U266 cells express MYCN instead of c-MYC).

### Western Blot Analysis

OPM2 cells (1.2×10⁶) were treated with Cpd 010 or DMSO control at indicated concentrations for 4 hours. Lysates were prepared in ice-cold RIPA lysis buffer (Thermofisher, Life Technologies) and supplemented with Halt protease phosphatase inhibitor cocktail (Thermofisher, Pierce Biotechnology). Protein concentrations of lysate samples were determined by bicinchoninic acid assay (BCA assay) using the Thermo Scientific Pierce BCA Protein Assay Kit (ThermoFisher, Life Technologies) as per manufacturer's instructions. Absorbance for protein concentration estimation was measured using a BMG OPTIMA (Fluostar optima, BMG Labtech). Lysates (25-50 μg) were separated on pre-cast gels (Bis-Tris 4-12% gradient gels) (Bolt, Life Technologies) and transferred onto polyvinylidene difluoride (PVDF) membranes (Immobilon-FL, Merck). The membranes were sequentially incubated with primary antibodies o.n. at 4° C.; mouse anti-β-actin (Sigma-Aldrich, clone AC-74), rabbit anti-cMyc (Cell Signaling, #9402S), rabbit anti-pAKT Ser473 (Cell Signaling, #9271), rabbit anti-pS6 Ser240/244 (Cell Signaling, #2215). Subsequently they were incubated with horse radish pe-roxidase (HRP)-conjugated secondary antibodies for 1-2 h at r.t before the detection of proteins by enzymatic (chemiluminescent) reaction using a ChemiDoc^{Tm} XRS+ System (Bio-Rad La-boratories Inc.). All Western Blots were repeated at least three times.

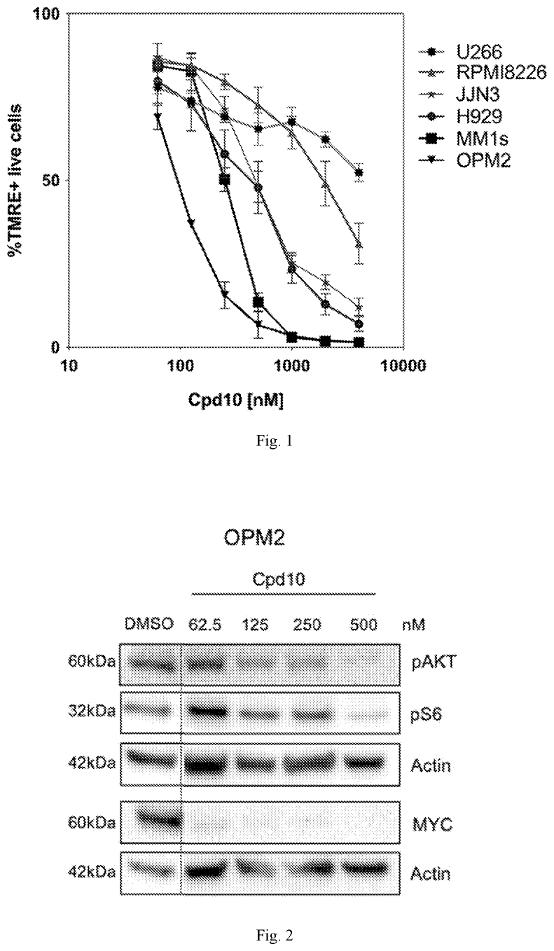

The results, shown in FIG. 2, demonstrate that Cpd 010 suppressed levels of both pAKT (PI3K substrate) and MYC (BET target protein) at all test concentrations.

### Cellular Thermal Shift Assay (CETSA)

Ramos cells (human Burkitt lymphoma cell line) at 20×10⁶ were washed in PBS followed by resuspension in 5 mL serum-free RPMI. They were treated either with Cpd 010 or DMSO at 20 μM for 40 min at 37° C. with 5% CO₂. Cells were then collected, resuspended in PBS and separated into PCR tubes. They were heated at designated temperature for strictly 3 min followed by another 3 min at 25° C. on ABI Applied Biosystems 9902 Veriti PCR Thermal Cycler. Immediately, they were placed in liquid nitrogen for snap freezing. The samples were freeze-thawed until the final samples were place on ice. They were centrifuged and supernatants were collected. Loading dye was mixed and these were processed as per our Western Blot protocol (see above). The following primary antibodies were used; rabbit BRD4 (Cell Signaling, #13440), rabbit BRD2 (Cell Signaling, #5848), and mouse anti-β-actin (Sigma-Aldrich, clone AC-74).

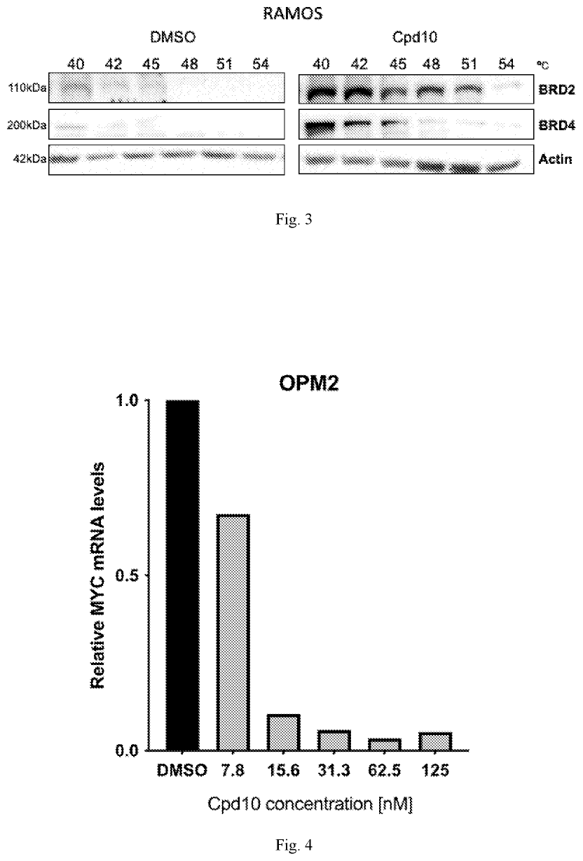

The results, shown in FIG. 3, demonstrate that Cpd 010 delayed clearance of both BRD2 and BRD4 proteins with increasing temperature as compared to DMSO control. This suggests tight binding and stabilisation of both target proteins by Cpd 010.

### RT-qPCR

OPM2 cells (1×10⁶) were treated with Cpd 010 or DMSO vehicle at indicated concentrations for 2 h. RNA was extracted from cell pellets using the PureLink™ RNA Mini Kit (ThermoFisher Scientific, #121839025) as per the manufacturer's instructions. cDNA was syn thesised with the SuperScript™ III cDNA synthesis kit (ThermoFisher Scientific, #18080093) according to the manufacturer's instructions. Quantitative PCR analysis of samples was per formed on the QuantStudio7 Flex RT-PCR system (ThermoFisher) with SYBR™ Master Mix (ThermoFisher Scientific, #4472903). L32 were used as the human control gene. Primer se quences were: Homo sapiens MYC F: GGACGACGAGACCTTCATCAA R: CCAGCTTCTCTGAGACGAGCTT, Homo sapiens L32 F: TTCCTGGTCCACAATGTCAAG R: TTGTGAGCGATCTCGGCAC. Each RT-qPCR am plification was performed in triplicate (technical triplicates) and repeated 2 times (biological replicates).

The results, shown in FIG. 4, demonstrate that Cpd 010 showed transcriptional downregulation of MYC gene, as demonstrated by significant decrease in MYC mRNA level.

RNA-Seq and gene Set Enrichment Analysis (GSEA)

OPM2 cells (4×10⁶) were treated with 250 nM of Cpd 010 or DMSO vehicle for 2 h. Cells pellets were collected, and RNA was extracted using the Nucleospin RNA extraction kit (Macherey-Nagel) as per the manufacturer's instructions. The QuantSeq 30 mRNA-seq Library Prep Kit for Illumina (Lexogen) was used to generate libraries as per the manufacturer's instructions, which were sequenced on the NextSeq (Illumina; 75 bp SE). GSEA2-2.2.2 was used for identification of enriched signatures obtained from the MSigDB Hallmarks datasets (Liber-zon et al., 2015). Samples were prepared in technical triplicates.

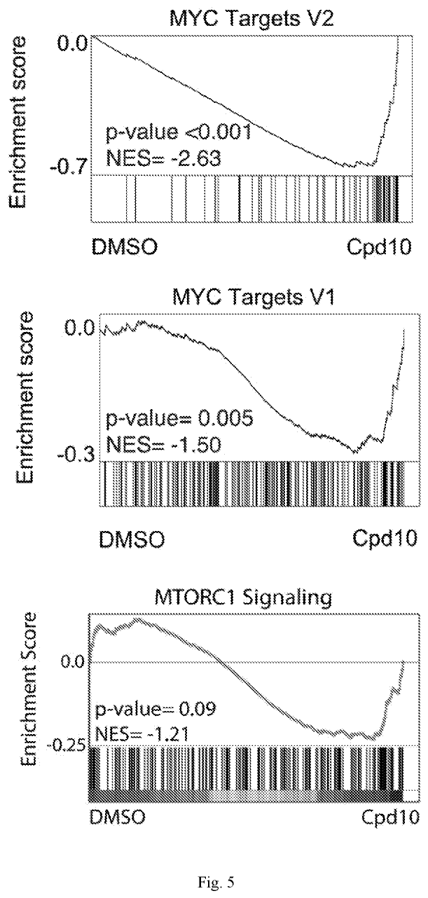

The results, shown in FIG. 5, show that Cpd 010 demonstrated enrichment against MYC target genes with a trend toward MTORC1 signaling genes.

### Chromatin Immunoprecipitation and Sequencing Analysis

OPM2 cells (4×10⁶) were treated with 250 nM of Cpd 010 or DMSO control for 2 h. Cells were cross-linked, and nuclei were lysed to achieve a mean DNA fragment size of 300-500 bp. Immunoprecipitation was performed using protein A/G magnetic beads for at least 12 h at 4° C. using the following antibodies: anti-BRD4 anti-H₃K₂₇Ac, anti-RNA polymerase II. Reverse crosslinking of DNA was performed, and DNA was purified using QIAquick PCR purification kit. The KAPA Hyper Prep Kit was used to generate libraries as per the manufacturer's instruction, which sequenced on the NextSeq (Illumina; 75 bp SE).

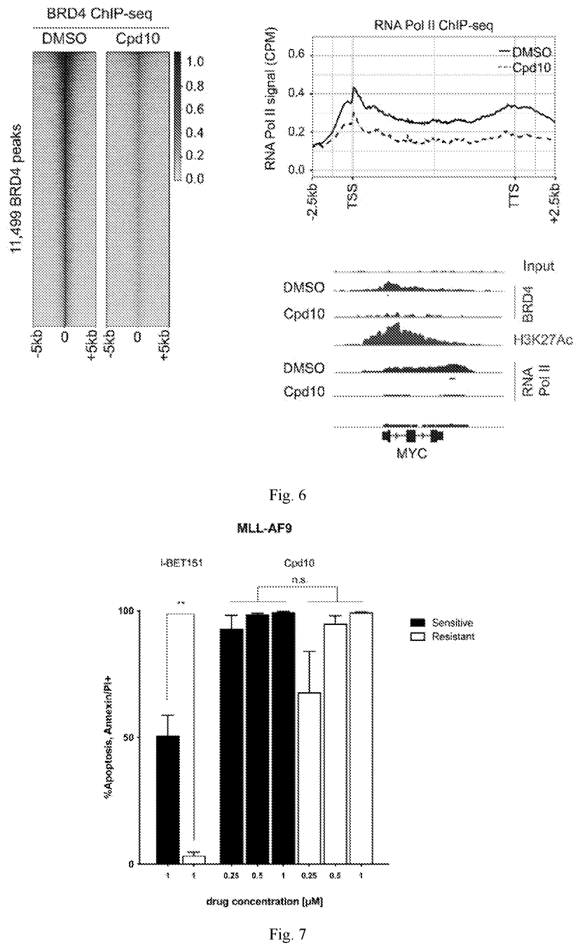

The results, shown in FIG. 6, demonstrate that Cpd 010 demonstrated robust displace ment of BRD4. At MYC locus, significant reduction in BRD4 and RNA Pol II was observed after the treatment with Cpd 010.

### Apoptosis Assay in BET-Inhibitor-Resistant Cells

Mouse AML cells; MLL-AF9 (IBET-151-sensitive and IBET-151-resistant) (5×10⁴) were treated with indicated compounds for 72 h. Apoptotic analysis was performed by flow cytometry using Annexin V/propidium iodide (PI) stains. Data were collected on a BD LSR-Fortessa X20 (BD Biosciences) and analysed using FlowJo Software, version 10.0.7 (Tree Star). IC₅₀ were calculated using the GraphPad Prism Software Version 7.0c. Data are representative of biological triplicates.

The results, as shown in FIG. 7, demonstrate that Cpd 010 was capable of inducing apoptosis in I-BET151-resistant MLL-AF9 cells, comparable to the I-BETE151-sensitive cells. At the same time, resistance to I-BET151 drug in the resistant cells was confirmed using 1 μM I-BET151.

### Synergy Between PI3K and BET Inhibitors and Synergy Score Calculation

An apoptosis assay using TMRE stain by flow cytometry was performed in OPM2 cells with indicated compounds. Cpd 034 is a selective PI3K inhibitor, and Cpd 035 is a selective BRD-4 inhibitor. Using Synergyfinder web application (https://synergyfinder.fimm.fi), synergy score was calculated. Zero Interaction Potency (ZIP) reference models was utilised.

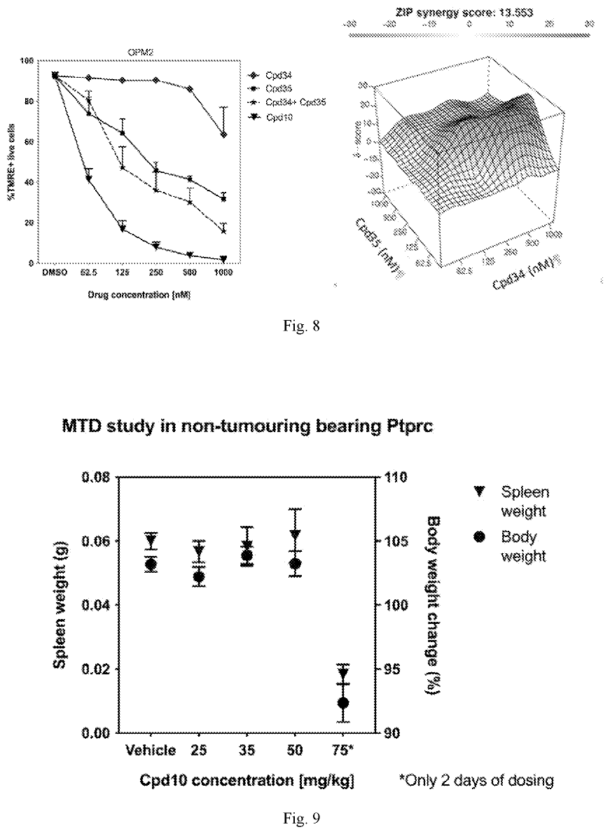

The results, shown in FIG. 8, indicate that a combination of Cpd 034 and Cpd 035 showed synergistic induction of apoptosis compared to each drug alone. Synergy score, which is a deviation between observed and expected responses (positive score indicates synergy; negative indicates antagonism), was 13.553. Cpd 010 showed even more enhanced apoptosis compared to the combination of two drugs together.

### In Vivo Assessment of Maximum Tolerated Dose (MTD)

The Peter MacCallum Cancer Centre Animal Ethics Committee approved all in vivo procedures in this study. B6.SJL-Ptprc<a>Pepc<b>/BoyJ (B6.CD45.1) mice were purchased from the Walter and Eliza Hall Institute (Melbourne, VIC). For enhanced solubility, Cpd 010 was reconstituted in double dose with 60% Hydroxypropyl-B-cyclodextrin (HPBCD; Cyclodextrin Technologies Development Inc.) and subsequently diluted with ultrafiltered water in 1:1 ratio prior to injection. Vehicle control was 60% HPBCD subsequently diluted 1:1 ratio with ultrafiltered water prior to injection for the final concentration of 30% HPBCD. MTD was carried out in 6-8 week old, non-tumour bearing female mice starting from 25 mg/kg of Cpd 010, 5 day intraperitoneal injection. Six mice received Cpd 010 and two mice received vehicle (30% HPBCD). Full Blood Examination (FBE), serum, spleen weight and morphological examination for toxicity were assessed on day 8 and day 15. In the absence of toxicity further dose escalation was carried out.

The results, shown in FIG. 9, demonstrate that the MTD for Cpd 010 was determined to be 50 mg/kg. At this dose, there were no changes in spleen and body weight. FBE at day 8 showed moderate thrombocytopenia at 50 mg/kg however, on day 15 they have recovered to the normal level.

### In Vivo Therapeutic Assessment of Cpd 010

For transplantation of Eμ-Myc lymphomas in vivo, cohorts of 6-8 week old B6.SJL-Ptprc<a>Pepc<b>/BoyJ (B6.CD45.1) mice were inoculated via tail vein injection with 5×10⁵ Eμ-Myc lymphoma cells (C57BL/6 (CD45.2)). Cpd 010 and vehicle controls (30% HPBCD) were prepared as above. Three days after lymphoma inoculation, intraperitoneal (IP) injection of compound 10 (45 mg/kg), or vehicle (30% HPBCD) was administered 5 days per week, for a total of 3-weeks of therapy. Detection of tumour cells on peripheral blood was carried out by flow cytometric analysis using the following antibodies; fixable Viability dye-AmCyan (Zom-bie Aqua™ Fixable Viability Kit, BioLegend), anti-mouse CD19-PE (ThermoFisher Scientific, #12-0193-82), anti-mouse CD45R (B220)-APC (ThermoFisher Scientific, #RA3-6B2), anti-mouse CD45.1-PerCP-Cy™5.5 (ThermoFisher Scientific, #A20), anti-mouse CD45.2-FITC (BioLegend, #104). Data were collected on a BD LSR-Fortessa X20 (BD Biosciences). The data were analysed using FlowJo Software, version 10.0.7 (Tree Star).

Cpd 010 showed statistically significant survival benefit (p<0.0001) with 90% demon-strating curative response.

For Vk*MYC multiple myeloma mouse models, wild type C₅₇/B16 mice were irradi-ated with 2 fractions of 3.3Gy (Gammacell® 40 caesium source, Atomic Energy of Canada Ltd., Ontario, Canada) prior to transplantation of Vk*MYC myeloma cells (2.4×10⁵). Mice were monitored for tumour progression by measuring M-proteins via serum protein electropho-resis (SPEP). Mice were treated with 3 weeks of Cpd 010 or vehicle once daily (5 days a week) for 3 weeks when their average SPEP has reached 7-10%. Serial measurement of SPEP was carried out during and post therapy. Overall survival was recorded.

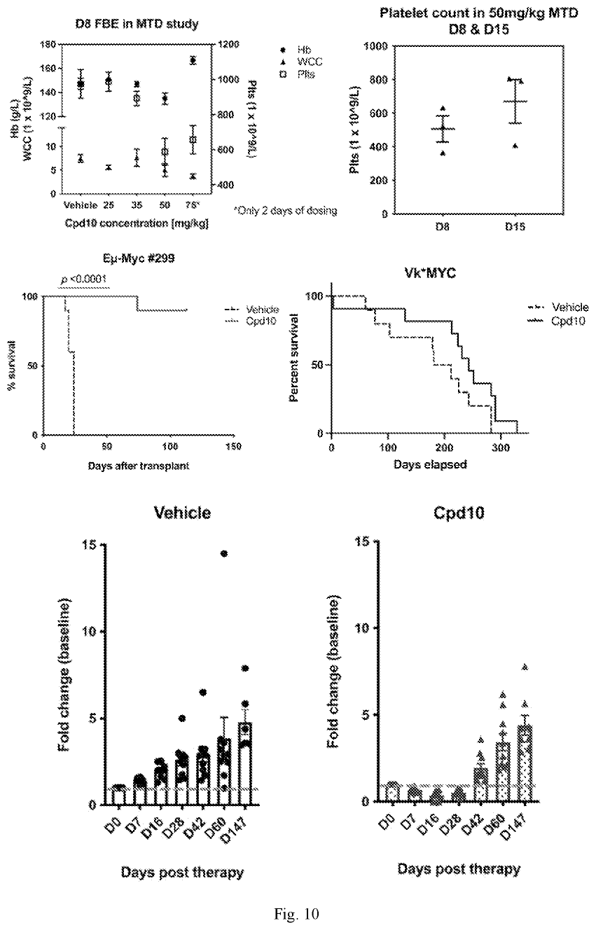

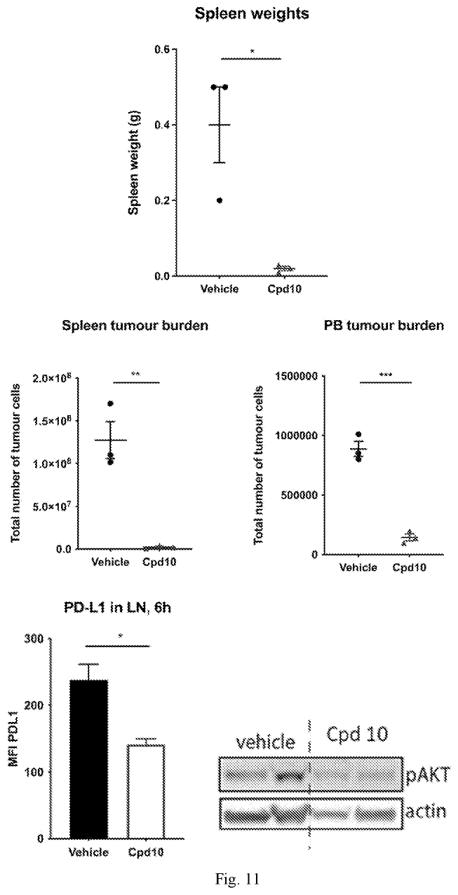

The results, shown in FIG. 10, demonstrate that median survival between the mice treated with Cpd 010 and vehicle was 243 vs. 196.5 days, respectively. Cpd 010 was able to reduce tumour burden (M-proteins) while the mice were on treatment, three mice even cleared the M-proteins completely during treatment.

In Vivo Assessment of PD-L1, PI3K Activity and Tumour Burden from a Single Dose

B6.SJL-Ptprc<a>Pepc<b>/BoyJ (B6.CD45.1) mice were inoculated via tail vein injection with 5×10⁵ Eμ-Myc lymphoma cells and left untreated for 12-14 days. A single injection of Cpd 010 at 35-45 mg/kg, or vehicle control (30% HPBCD) was administered. Half of the mice (n=6) were harvested 6 h post injection while the other half (n=6) were harvested 16 h later. Assessment of tumour burden was carried out on single cell suspension of harvested lymph nodes, spleen and blood by flow cytometry using antibodies as above. PD-L1 MFI was assessed by flow cytometry in harvested LNs using anti-mouse PD-L1-PE (ThermoFisher Scientific, clone MIH5) or isotype control antibodies (Mouse IgG2b PE, #559529). Harvested LNs were also washed in PBS and lysates were collected using Laemmli Buffer (AlfaAeser #J61337). They were processed as per our Western Blot protocol (see above) before they were probed for primary pAKT antibodies. Spleen size and weights were also measured between the treatment groups.

The results, shown in FIG. 11, demonstrate that Cpd 010 demonstrated marked reduction in number of tumour cells in peripheral blood and harvested spleen at 16 h. The size of the spleen, which was enlarged due to tumour invasion, was also significantly reduced by Cpd 010 after single dose of treatment.

In terms of target engagement, PD-L1 expression in the tumour cells were measured in the harvested LN and spleen. As expression of the Myc transgene in Eμ-Myc is not BET-de pendent, MYC cannot be measured as a read-out of BET inhibition, therefore PD-L1 was used as an indicator for BET inhibition. In the LN, PD-L1 expression was significantly reduced with Cpd 010. Similarly, pAKT (PI3K substrate) was suppressed by Cpd 010 in the lysates made from harvested LN.

1. A compound of Formula (I), or a pharmaceutically acceptable salt, solvate or stereoisomer thereof: 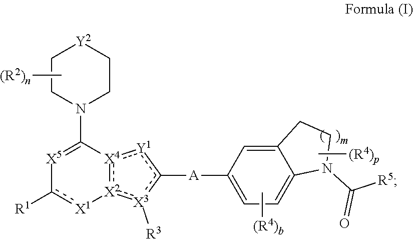
wherein
X¹ is C(R) or N;
X² is CH, C or N;
X³ is C or N;
X⁴ is C or N;
X⁵ is C or N;
Y¹ is CH, N or S;
Y² is O, CH₂, or N(R);
R is selected from the group consisting of hydrogen, halogen, and C₁₋₆alkyl;
R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
m is 1, 2, or 3;
n is 0, 1, 2, 3, 4, 5, 6, 7, or 8;
p is 0, 1, 2, 3, 4, 5, 6, 7, or 8;
b is 0, 1, 2, or 3;
A is a linker of Formula (II):
-[A¹]ᵤ₁-[Z¹]ₜ₁-[A²]ᵤ₂-[Z²]ₜ₂-[A³]ᵤ₃-[Z³]ₜ₃— Formula (II);
wherein A¹, A², and A³ are each independently selected from the group consisting of —C₁₋₆alkylene-, —C(O)—, —C(O)N(R¹¹)—, —N(R¹¹)C(O)—, —N(R¹¹)—, 3-10 membered carbocyclyl, and 3-10 membered heterocyclyl; wherein the —C₁₋₆alkylene, 3-10 membered carbocyclyl, and 3-10 membered heterocyclyl, are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
u¹, u², and u³ are each independently 0, 1, 2, 3, 4, or 5;
t¹, t², and t³ are each independently 0, 1, or 2; and
wherein at least one of u¹, u², u³, t¹, t², and t³, is at least 1; and
if present, R⁶ and R⁷ are each independently selected from the group consisting of hydrogen, —C₁₋₆alkyl, 3-10 membered carbocyclyl, and 3-10 membered heterocyclyl; and
if present, R¹¹ are each independently selected from the group consisting of hydrogen, hydroxy, —C₁₋₆alkyl, —C(O)OC₁₋₆alkyl, —C(O)C₁₋₆alkyl, 3-10 membered carbocyclyl, and 3-10 membered heterocyclyl; and wherein —C₁₋₆alkyl is unsubstituted or substituted with one or more substituents each independently selected from the group consisting of —OH, ═O, halogen, —O—C₁₋₆alkyl, —NH₂, —N(H)(C₁₋₆alkyl), —N(C₁₋₆alkyl)₂, —C(O)NH₂, —C(O)N(H)(C₁₋₆alkyl), —C(O)N(C₁₋₆alkyl)₂, —C(O)OH, —C(O)OC₁₋₆alkyl, and —C(O)C₁₋₆alkyl.

2. The compound of claim 1, or a pharmaceutically acceptable salt, solvate or stereoisomer thereof, wherein Formula (I) is a compound of Formula (Ia): 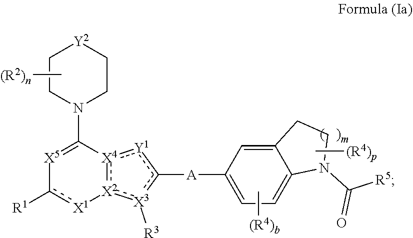
wherein
X¹ is C(R) or N;
X² is CH, C or N;
X³ is C or N;
X⁴ is C or N;
X⁵ is C or N;
Y¹ is CH, N or S;
Y² is O, CH₂, or N(R);
R is selected from the group consisting of hydrogen, halogen, and C₁₋₆alkyl;
R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
m is 1, 2, or 3;
n is 0, 1, 2, 3, 4, 5, 6, 7, or 8;
p is 0, 1, 2, 3, 4, 5, 6, 7, or 8;
b is 0, 1, 2, or 3;
A is a linker of Formula (IIa): 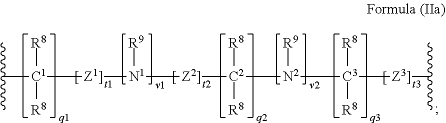
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

3. The compound, salt, solvate or stereoisomer of claim 1 or claim 2, wherein Formula (I) is a compound of Formula (Ib): 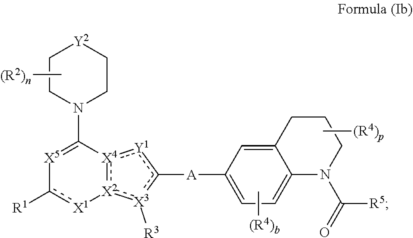
wherein
X¹ is C(R) or N;
X² is CH, C or N;
X³ is C or N;
X⁴ is C or N;
X⁵ is C or N;
Y¹ is CH, N or S;
Y² is O, CH₂, or N(R);
R is selected from the group consisting of hydrogen, halogen, and C₁₋₆alkyl;
R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
n is 0, 1, 2, 3, 4, 5, 6, 7, or 8;
p is 0, 1, 2, 3, 4, 5, 6, 7, or 8;
b is 0, 1, 2, or 3;
A is a linker of Formula (IIa): 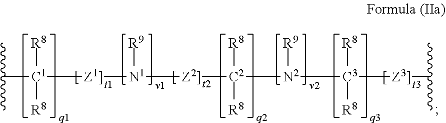
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

4. The compound, salt, solvate or stereoisomer of any one of claims 1 to 3, wherein Formula (I) is a compound of Formula (Ibi): 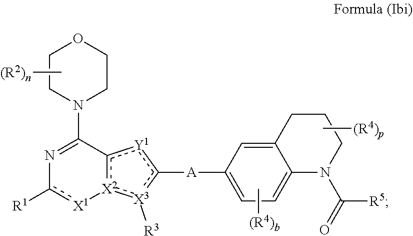
wherein
X¹ is C(R) or N;
X² is CH, C or N;
X³ is C or N;
Y¹ is CH, N or S;
R is selected from the group consisting of hydrogen, halogen, and C₁₋₆alkyl;
R¹, R², R³, and R⁴ are each independently selected from the group consisting of hydro gen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R′), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
n is 0, 1, 2, 3, 4, 5, 6, 7, or 8;
p is 0, 1, 2, 3, 4, 5, 6, 7, or 8;
b is 0, 1, 2, or 3;
A is a linker of Formula (IIa): 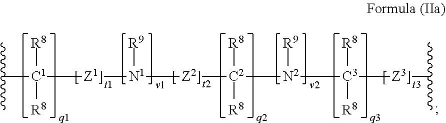
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

5. The compound, salt, solvate or stereoisomer of any one of claims 1 to 4, wherein Formula (I) is a compound of Formula (Ic): 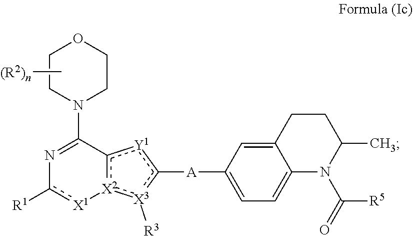
wherein
X¹ is CH or N;
X² is CH, C or N;
X³ is C or N;
Y¹ is CH, N or S;
R¹, R², and R³ are each independently selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
R⁵ is selected from the group consisting of —C₁₋₆alkyl, —O(R⁶), 3-10 membered carbocyclyl, and 3-10 membered heterocyclyl;
n is 0, 1, 2, 3, 4, 5, 6, 7, or 8;
A is a linker of Formula (IIa): 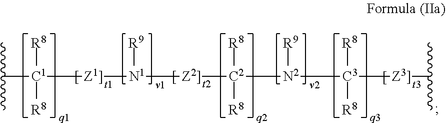
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

6. The compound, salt, solvate or stereoisomer of any one of claims 1 to 5, wherein Formula (I) is a compound of Formula (Ic): 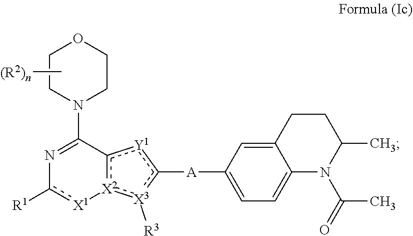
wherein
X¹ is CH or N;
X² is CH, C or N;
X³ is C or N;
Y¹ is CH, N or S;
R¹, R², and R³ are each independently selected from the group consisting of hydrogen, halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —OR⁶, —N(R⁶)(R⁷), —C(O)R⁶, —C(O)OR⁶, —C(O)N(R⁶)(R⁷), and —N(R⁶)C(O)(R⁷);
wherein said alkyl, alkenyl, alkynyl, carbocyclyl or heterocyclyl are each unsubstituted or substituted with one or more substituents each independently selected from the group consisting of halogen, —C₁₋₆alkyl, —C₁₋₆haloalkyl, —OR⁶, —N(R⁶)(R⁷), —C(O)N(R⁶)(R⁷), —N(R⁶)C(O)(R⁷), —C(O)OR⁶, and —C(O)R⁶; and
wherein each —C₁₋₆alkyl is unsubstituted or substituted with one or more substituents each independently selected from —OR⁶, —N(R⁶)(R⁷), —C(O)N(R⁶)(R′), —N(R⁶)C(O)(R⁷), —C(O)OR⁶, and —C(O)R⁶;
n is 0, 1, 2, 3, 4, 5, 6, 7, or 8;
A is a linker of Formula (IIa): 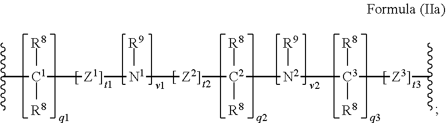
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹, Z², and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R′), —N(R⁶)C(O)(R⁷), —C(O)OR⁶, and —C(O)R⁶;
q¹, q², and q³ are each independently 0, 1, 2, 3, 4, or 5;
t¹, t², and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

7. The compound, salt, solvate or stereoisomer of any one of claims 1 to 6, wherein Formula (II) is selected from the group consisting of Formula (IIai) and Formula (Ilaii): 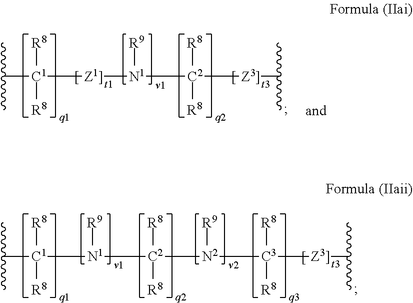
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹ and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶, and —C(O)R⁶;
q¹, q², and q³ are each independently 0, 1, 2, 3, 4, or 5;
t¹ and t³ are each independently 0, 1, or 2;
v¹ and v² are each independently 0, 1 or 2; and
wherein at least one of q¹, q², q³, t¹, t², t³, v¹, and v², is at least 1; and
if present, R⁶ and R⁷ are each independently selected from the group consisting of hydrogen, —C₁₋₆alkyl, 3-10 membered carbocyclyl, and 3-10 membered heterocyclyl.

8. The compound, salt, solvate or stereoisomer of any one of claims 1 to 7, wherein Formula (II) is selected from the group consisting of Formula (Ilaiii) and Formula (Ilaiv): 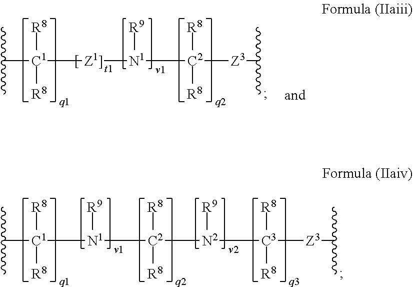
wherein R⁸ and R⁹ are each independently selected from the group consisting of hy drogen, —OH, —C₁₋₆alkyl, —C(O)OC₁₋₆alkyl, and —C(O)C₁₋₆alkyl; or two R⁸ are taken together to form a C═O substituent with the adjacent carbon atom; wherein the —C₁₋₆alkyl is unsubstituted or substituted with one or more substituents independently selected from the group consisting of halogen, —OH, ═O, —O—C₁₋₆alkyl, —N(R⁶)(R⁷), —C(O)N(R⁶)(R⁷), —N(R⁶)C(O)(R⁷), —C(O)OR⁶, and —C(O)R⁶;
C¹, C², and C³ are each carbon;
N¹ and N² are each nitrogen;
Z¹ and Z³ are each independently selected from the group consisting of 3-10 membered carbocyclyl and 3-10 membered heterocyclyl; wherein the carbocyclyl and heterocyclyl are each unsubstituted or substituted with one or more substituents independently selected from the group consisting of halogen, —OH, ═O, —C₁₋₆alkyl, —O—C₁₋₆alkyl, —N(R⁶)(R⁷), —C(O)N(R⁶)(R⁷), —N(R⁶)C(O)(R⁷), —C(O)OR⁶ and —C(O)R⁶;
q¹, q², and q³ are each independently 0, 1, 2, 3, 4, or 5;
t¹ is 0, 1, or 2;
v¹ and v² are each independently 0, 1 or 2; and
if present, R⁶ and R⁷ are each independently selected from the group consisting of hydrogen, —C₁₋₆alkyl, 3-10 membered carbocyclyl, and 3-10 membered heterocyclyl.

9. The compound, salt, solvate or stereoisomer of any one of claims 1 to 8, wherein t¹ is 1, and Z¹ is Formula (III): 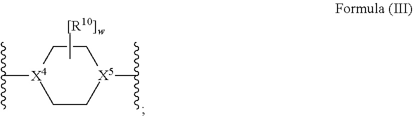
wherein
X⁴ is CH or N;
X⁵ is CH or N;
R¹⁰ are each independently selected from the group consisting of halogen, —C₁₋₆alkyl, —O—C₁₋₆alkyl, —C₁₋₆haloalkyl, —O—C₁₋₆haloalkyl, —C₂₋₆alkenyl, —C₂₋₆alkynyl, 3-10 membered carbocyclyl, 3-10 membered heterocyclyl, —CN, —OR⁶, —SR⁶, —N(R⁶)(R⁷), —C(O)R⁶, —C(O)OR⁶, —C(O)N(R⁶)(R⁷), —N(R⁶)C(O)R⁷, —S(O)OR⁶, —S(O)ON(R⁶)(R⁷), and —N(R⁶)S(O)OR⁷;
w is 0, 1, 2, 3, 4, 5, 6, 7, or 8; and
if present, R⁶ and R⁷ are each independently selected from the group consisting of hydrogen, —C₁₋₆alkyl, 3-10 membered carbocyclyl, and 3-10 membered heterocyclyl.

10. The compound, salt, solvate or stereoisomer of claim 9, wherein w is 0.

11. The compound, salt, solvate or stereoisomer of any one of claims 1 to 10, wherein b is 0.

12. The compound, salt, solvate or stereoisomer of any one of claims 1 to 11, wherein X⁴ is C.

13. The compound, salt, solvate or stereoisomer of any one of claims 1 to 12, wherein X⁵ is C.

14. The compound, salt, solvate or stereoisomer of any one of claims 1 to 13, wherein Y² is O.

15. The compound, salt, solvate or stereoisomer of any one of claims 2 to 14, wherein q¹ is 1, and the two R⁸ of C¹ are hydrogen, or the two R⁸ of C¹ are taken together form a C═O substituent with the adjacent C¹ atom.

16. The compound, salt, solvate or stereoisomer of any one of claims 1 to 15, wherein Z³ is a 3-10 membered unsaturated heterocyclyl or a 3-10 membered unsaturated carbocyclyl.

17. The compound, salt, solvate or stereoisomer of any one of claims 1 to 16, wherein Z³ is selected from the group consisting of:

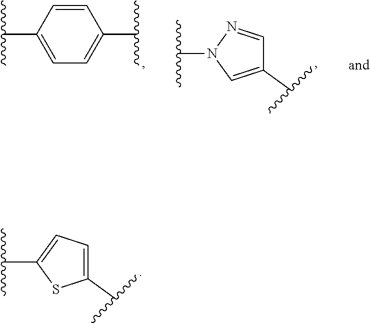

18. The compound, salt, solvate or stereoisomer of any one of claims 1 to 17, wherein X¹ is CH, X² is N, X³ is C, and Y¹ is N.

19. The compound, salt, solvate or stereoisomer of any one of claims 1 to 17, wherein X¹ is N, X² is C, X³ is C, and Y¹ is S.

20. The compound, salt, solvate or stereoisomer of any one of claims 1 to 17, wherein X¹ is CH, X² is C, X³ is N, and Y¹ is CH, and R³ is not present.

21. The compound, salt, solvate or stereoisomer of any one of claims 1 to 17, wherein X¹ is N, X² is N, X³ is C, and Y¹ is CH.

22. The compound, salt, solvate or stereoisomer of any one of claims 1 to 21, wherein R³ is hydrogen or —CH₃.

23. The compound, salt, solvate or stereoisomer of any one of claims 2 to 22, wherein if present, one or more R⁹ is hydrogen.

24. The compound, salt, solvate or stereoisomer of any one of claims 2 to 22, wherein if present, one or more R⁹ is —OH.

25. The compound, salt, solvate or stereoisomer of any one of claims 2 to 22, wherein if present, one or more R⁹ is —CH₃.

26. The compound, salt, solvate or stereoisomer of any one of claims 2 to 22, wherein if present, one or more R⁹ is —C₁₋₆alkyl, and each —C₁₋₆alkyl is independently substituted with one or more substituents selected from the group selected from —OH, ═O, and —NH₂.

27. The compound, salt, solvate or stereoisomer of any one of claims 1 to 26, wherein R¹ is selected from the group consisting of:

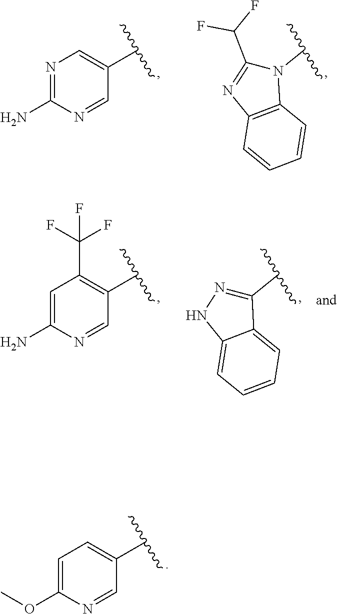

28. The compound, salt, solvate or stereoisomer of claim 1, wherein A is selected from the group consisting of:

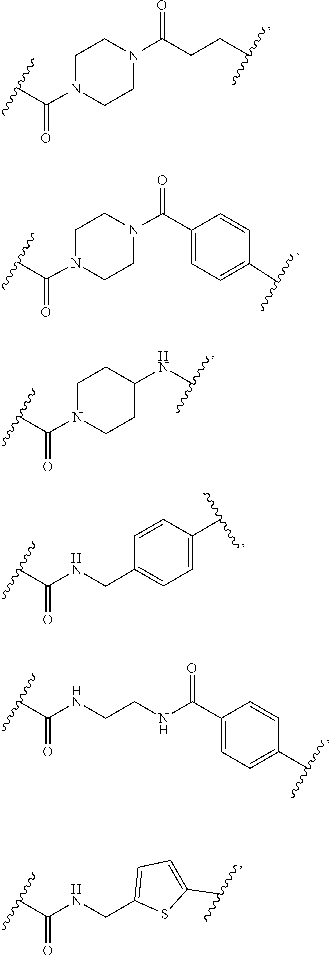 [SMILES: *CCC(=O)N1CCN(C(*)=O)CC1 ; *C(=O)N1CCN(C(=O)c2ccc(*)cc2)CC1 ; *NC1CCN(C(*)=O)CC1 ; *C(=O)NCc1ccc(*)cc1 ; *C(=O)NCCNC(=O)c1ccc(*)cc1 ; *C(=O)NCc1ccc(*)s1]

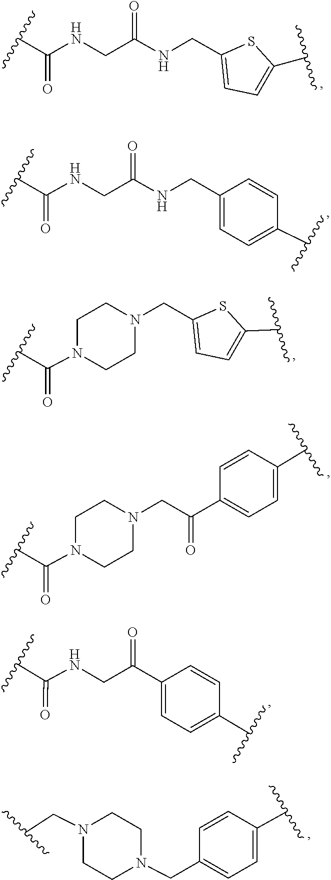 [SMILES: *C(=O)NCC(=O)NCc1ccc(*)s1 ; *C(=O)NCC(=O)NCc1ccc(*)cc1 ; *C(=O)N1CCN(Cc2ccc(*)s2)CC1 ; *C(=O)N1CCN(CC(=O)c2ccc(*)cc2)CC1 ; *C(=O)NCC(=O)c1ccc(*)cc1 ; *CN1CCN(Cc2ccc(*)cc2)CC1]

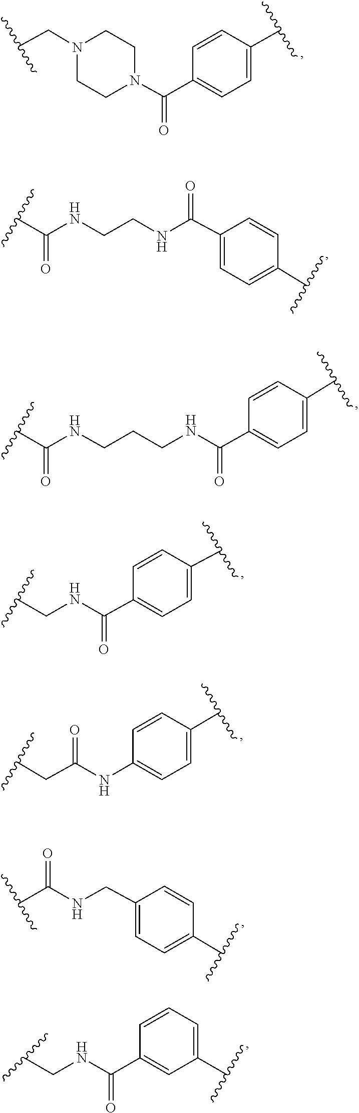

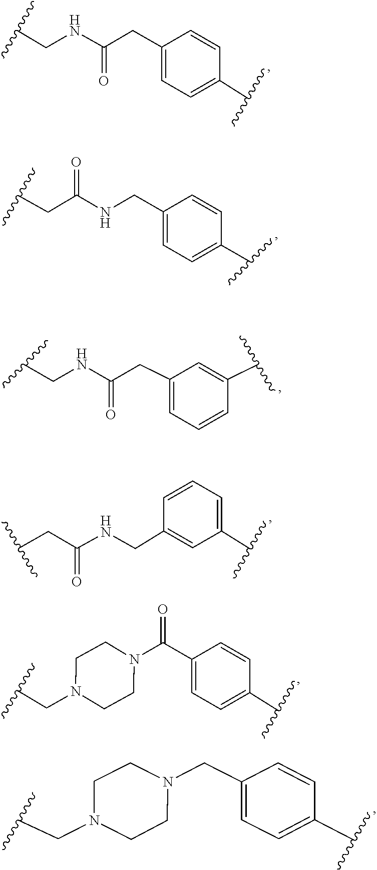 [SMILES: *CNC(=O)Cc1ccc(*)cc1 ; *CC(=O)NCc1ccc(*)cc1 ; *CNC(=O)Cc1cccc(*)c1 ; *CC(=O)NCc1cccc(*)c1 ; *CN1CCN(C(=O)c2ccc(*)cc2)CC1 ; *CN1CCN(Cc2ccc(*)cc2)CC1]

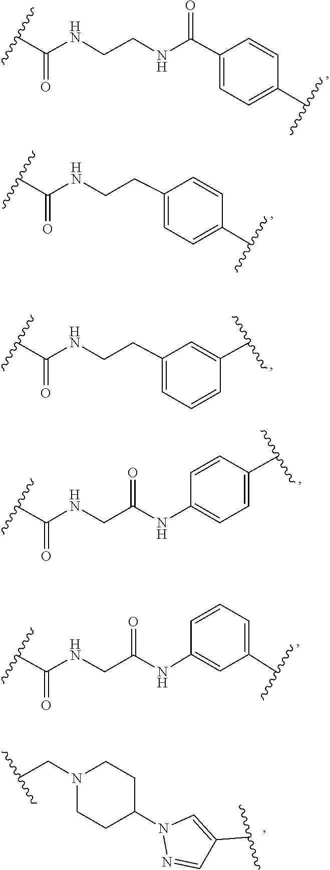 [SMILES: *C(=O)NCCNC(=O)c1ccc(*)cc1 ; *C(=O)NCCc1ccc(*)cc1 ; *C(=O)NCCc1cccc(*)c1 ; *C(=O)NCC(=O)Nc1ccc(*)cc1 ; *C(=O)NCC(=O)Nc1cccc(*)c1 ; *CN1CCC(n2cc(*)cn2)CC1]

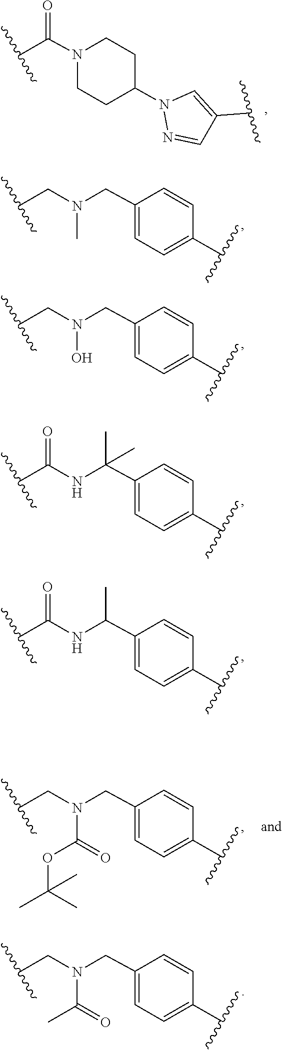

29. The compound, salt, solvate or stereoisomer of claim 1, wherein Formula (I) is selected from the group consisting of:
1-(6-(4-(4-(6-(2-aminopyrimidin-5-yl)-8-morpholinoimidazo[1,2-a]pyrazine-2-carbonyl)pi-perazine-1-carbonyl)phenyl)-2-methyl-3,4-dihydroquinolin-1(2H)-yl)ethan-1-one;
N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
N-(2-((4-(1-acetyl-2-methyl-1,2,3,4-tetrahydroquinolin-6-yl)benzyl)amino)-2-oxoethyl)-6-(2-aminopyrimidin-5-yl)-8-morpholinoimidazo[1,2-a]pyrazine-2-carboxamide;
1-(6-(5-((4-(6-(2-aminopyrimidin-5-yl)-8-morpholinoimidazo[1,2-a]pyrazine-2-carbonyl)pi-perazin-1-yl)methyl)thiophen-2-yl)-2-methyl-3,4-dihydroquinolin-1(2H)-yl)ethan-1-one;
N-((5-(1-acetyl-2-methyl-1,2,3,4-tetrahydroquinolin-6-yl)thiophen-2-yl)methyl)-6-(2-amino-pyrimidin-5-yl)-8-morpholinoimidazo[1,2-a]pyrazine-2-carboxamide;
N-(2-(((5-(1-acetyl-2-methyl-1,2,3,4-tetrahydroquinolin-6-yl)thiophen-2-yl)methyl)amino)-2-oxoethyl)-6-(2-aminopyrimidin-5-yl)-8-morpholinoimidazo[1,2-a]pyrazine-2-carboxamide;
1-(4-(1-acetyl-2-methyl-1,2,3,4-tetrahydroquinolin-6-yl)phenyl)-2-(4-(6-(2-aminopyrimidin-5-yl)-8-morpholinoimidazo[1,2-a]pyrazine-2-carbonyl)piperazin-1-yl)ethan-1-one;
N-(2-(4-(1-acetyl-2-methyl-1,2,3,4-tetrahydroquinolin-6-yl)phenyl)-2-oxoethyl)-6-(2-amino-pyrimidin-5-yl)-8-morpholinoimidazo[1,2-a]pyrazine-2-carboxamide;
N-(2-(4-(1-acetyl-2-methyl-1,2,3,4-tetrahydroquinolin-6-yl)benzamido)ethyl)-6-(2-aminopy-rimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(3-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(2-(4-(1-acetyl-2-methyl-1,2,3,4-tetrahydroquinolin-6-yl)benzamido)ethyl)-6-(2-amino-pyrimidin-5-yl)-8-morpholinoimidazo[1,2-a]pyrazine-2-carboxamide;
(S)-N-(3-(4-(1-acetyl-2-methyl-1,2,3,4-tetrahydroquinolin-6-yl)benzamido)propyl)-6-(2-ami-nopyrimidin-5-yl)-8-morpholinoimidazo[1,2-a]pyrazine-2-carboxamide;
(S)-1-(6-(4-(4-((6-(2-aminopyrimidin-5-yl)-8-morpholinoimidazo[1,2-a]pyrazin-2-yl)methyl)piperazine-1-carbonyl)phenyl)-2-methyl-3,4-dihydroquinolin-1(2H)-yl)ethan-1-one;
(S)-1-(6-(4-((4-((6-(2-aminopyrimidin-5-yl)-8-morpholinoimidazo[1,2-a]pyrazin-2-yl)methyl)piperazin-1-yl)methyl)phenyl)-2-methyl-3,4-dihydroquinolin-1(2H)-yl)ethan-1-one;
(S)-4-(1-acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide;
(S)-3-(1-acetyl-2-methyl-1,2,3,4-tetrahydroquinolin-6-yl)-N-((6-(2-aminopyrimidin-5-yl)-8-morpholinoimidazo[1,2-a]pyrazin-2-yl)methyl)benzamide;
(S)-2-(4-(1-acetyl-2-methyl-1,2,3,4-tetrahydroquinolin-6-yl)phenyl)-N-((6-(2-aminopyrim-idin-5-yl)-8-morpholinoimidazo[1,2-a]pyrazin-2-yl)methyl)acetamide;
(S)-2-(3-(1-acetyl-2-methyl-1,2,3,4-tetrahydroquinolin-6-yl)phenyl)-N-((6-(2-aminopyrim-idin-5-yl)-8-morpholinoimidazo[1,2-a]pyrazin-2-yl)methyl)acetamide;
(S)-N-(4-(1-acetyl-2-methyl-1,2,3,4-tetrahydroquinolin-6-yl)benzyl)-6-(2-aminopyrimidin-5-yl)-3-methyl-8-morpholinoimidazo[1,2-a]pyrazine-2-carboxamide;
(S)-N-(2-(4-(1-acetyl-2-methyl-1,2,3,4-tetrahydroquinolin-6-yl)benzamido)ethyl)-6-(2-amino-pyrimidin-5-yl)-3-methyl-8-morpholinoimidazo[1,2-a]pyrazine-2-carboxamide;
(S)-N-(4-(1-acetyl-2-methyl-1,2,3,4-tetrahydroquinolin-6-yl)phenyl)-2-(6-(2-aminopyrimidin-5-yl)-8-morpholinoimidazo[1,2-a]pyrazin-2-yl)acetamide;
(S)-N-(3-(1-acetyl-2-methyl-1,2,3,4-tetrahydroquinolin-6-yl)phenyl)-2-(6-(2-aminopyrimidin-5-yl)-8-morpholinoimidazo[1,2-a]pyrazin-2-yl)acetamide;
(S)-N-(4-(1-acetyl-2-methyl-1,2,3,4-tetrahydroquinolin-6-yl)benzyl)-2-(6-(2-aminopyrimidin-5-yl)-8-morpholinoimidazo[1,2-a]pyrazin-2-yl)acetamide;
(S)-N-(3-(1-acetyl-2-methyl-1,2,3,4-tetrahydroquinolin-6-yl)benzyl)-2-(6-(2-aminopyrimidin-5-yl)-8-morpholinoimidazo[1,2-a]pyrazin-2-yl)acetamide;
(S)-4-(1-acetyl-2-methyl-1,2,3,4-tetrahydroquinolin-6-yl)-N-((2-(2-aminopyrimidin-5-yl)-4-morpholinothieno[3,2-d]pyrimidin-6-yl)methyl)benzamide;
(S)-2-(4-(1-acetyl-2-methyl-1,2,3,4-tetrahydroquinolin-6-yl)phenyl)-N-((2-(2-aminopyrim-idin-5-yl)-4-morpholinothieno[3,2-d]pyrimidin-6-yl)methyl)acetamide;
(S)-1-(6-(4-(4-((2-(2-aminopyrimidin-5-yl)-4-morpholinothieno[3,2-d]pyrimidin-6-yl)methyl)piperazine-1-carbonyl)phenyl)-2-methyl-3,4-dihydroquinolin-1(2H)-yl)ethan-1-one;
(S)-1-(6-(4-((4-((2-(2-aminopyrimidin-5-yl)-4-morpholinothieno[3,2-d]pyrimidin-6-yl)methyl)piperazin-1-yl)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)benzyl)-2-(2-aminopyrimidin-5-yl)-4-morpholinothieno[3,2-d]pyrimidine-6-carboxamide;
(S)-N-(2-(4-(1-acetyl-2-methyl-1,2,3,4-tetrahydroquinolin-6-yl)benzamido)ethyl)-2-(2-amino-pyrimidin-5-yl)-4-morpholinothieno[3,2-d]pyrimidine-6-carboxamide;
(S)-N-(4-(1-acetyl-2-methyl-1,2,3,4-tetrahydroquinolin-6-yl)benzyl)-2-(2-aminopyrimidin-5-yl)-7-methyl-4-morpholinothieno[3,2-d]pyrimidine-6-carboxamide;
(S)-N-(2-(4-(1-acetyl-2-methyl-1,2,3,4-tetrahydroquinolin-6-yl)benzamido)ethyl)-2-(2-amino-pyrimidin-5-yl)-7-methyl-4-morpholinothieno[3,2-d]pyrimidine-6-carboxamide;
(R)-N-(4-(1-acetyl-2-methyl-1,2,3,4-tetrahydroquinolin-6-yl)benzyl)-6-(2-aminopyrimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-8-(piperazin-1-yl)imidazo[1,2-a]pyrazine-2-carboxamide hydrochloride;
(S)-N-(2-(4-(1-acetyl-2-methyl-1,2,3,4-tetrahydroquinolin-6-yl)-N-methylbenzamido)ethyl)-6-(2-aminopyrimidin-5-yl)-N-methyl-8-morpholinoimidazo[1,2-a]pyrazine-2-carboxamide;
(S)-N-(4-(1-acetyl-2-methyl-1,2,3,4-tetrahydroquinolin-6-yl)benzyl)-6-(2-aminopyrimidin-5-yl)-N-methyl-8-morpholinoimidazo[1,2-a]pyrazine-2-carboxamide;
(S)-N-(4-(1-acetyl-2-methyl-1,2,3,4-tetrahydroquinolin-6-yl)benzyl)-2-(2-aminopyrimidin-5-yl)-N-methyl-4-morpholinothieno[3,2-d]pyrimidine-6-carboxamide;
(S)-N-(4-(1-acetyl-2-methyl-1,2,3,4-tetrahydroquinolin-6-yl)benzyl)-2-(2-aminopyrimidin-5-yl)-N,7-dimethyl-4-morpholinothieno[3,2-d]pyrimidine-6-carboxamide;
(S)-N-(3-(4-(1-acetyl-2-methyl-1,2,3,4-tetrahydroquinolin-6-yl)benzamido)propyl)-2-(2-ami-nopyrimidin-5-yl)-4-morpholinothieno[3,2-d]pyrimidine-6-carboxamide;
(S)-N-(3-(4-(1-acetyl-2-methyl-1,2,3,4-tetrahydroquinolin-6-yl)benzamido)propyl)-2-(2-ami-nopyrimidin-5-yl)-7-methyl-4-morpholinothieno[3,2-d]pyrimidine-6-carboxamide;
(S)-1-(6-(4-((((2-(2-aminopyrimidin-5-yl)-4-morpholinothieno[3,2-d]pyrimidin-6-yl)methyl)(methyl)amino)methyl)phenyl)-2-methyl-3,4-dihydroquinolin-1(2H)-yl)ethan-1-one;
(S)-1-(6-(4-((((2-(2-aminopyrimidin-5-yl)-7-methyl-4-morpholinothieno[3,2-d]pyrimidin-6-yl)methyl)(methyl)amino)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)phenethyl)-6-(2-aminopyrimidin-5-yl)-8-morpholinoimidazo[1,2-a]pyrazine-2-carboxamide;
(S)-N-(4-(1-acetyl-2-methyl-1,2,3,4-tetrahydroquinolin-6-yl)phenethyl)-2-(2-aminopyrimidin-5-yl)-4-morpholinothieno[3,2-d]pyrimidine-6-carboxamide;
(S)-N-(4-(1-acetyl-2-methyl-1,2,3,4-tetrahydroquinolin-6-yl)phenethyl)-2-(2-aminopyrimidin-5-yl)-7-methyl-4-morpholinothieno[3,2-d]pyrimidine-6-carboxamide;
(S)-N-(2-((4-(1-acetyl-2-methyl-1,2,3,4-tetrahydroquinolin-6-yl)phenyl)amino)-2-oxoethyl)-2-(2-aminopyrimidin-5-yl)-7-methyl-4-morpholinothieno[3,2-d]pyrimidine-6-carboxamide;
(S)-N-(2-((4-(1-acetyl-2-methyl-1,2,3,4-tetrahydroquinolin-6-yl)phenyl)amino)-2-oxoethyl)-6-(2-aminopyrimidin-5-yl)-3-methyl-8-morpholinoimidazo[1,2-a]pyrazine-2-carboxamide;
(S)-N-(2-((3-(1-acetyl-2-methyl-1,2,3,4-tetrahydroquinolin-6-yl)phenyl)amino)-2-oxoethyl)-2-(2-aminopyrimidin-5-yl)-7-methyl-4-morpholinothieno[3,2-d]pyrimidine-6-carboxamide;
(S)-N-(3-(1-acetyl-2-methyl-1,2,3,4-tetrahydroquinolin-6-yl)phenethyl)-6-(2-aminopyrimidin-5-yl)-3-methyl-8-morpholinoimidazo[1,2-a]pyrazine-2-carboxamide;
(S)-N-(3-(1-acetyl-2-methyl-1,2,3,4-tetrahydroquinolin-6-yl)phenethyl)-2-(2-aminopyrimidin-5-yl)-7-methyl-4-morpholinothieno[3,2-d]pyrimidine-6-carboxamide;
(S)-1-(6-(1-(1-((2-(2-aminopyrimidin-5-yl)-7-methyl-4-morpholinothieno[3,2-d]pyrimidin-6-yl)methyl)piperidin-4-yl)-1H-pyrazol-4-yl)-2-methyl-3,4-dihydroquinolin-1(2H)-yl)ethan-1-one;
(S)-1-(6-(1-(1-(2-(2-aminopyrimidin-5-yl)-7-methyl-4-morpholinothieno[3,2-d]pyrimidine-6-carbonyl)piperidin-4-yl)-1H-pyrazol-4-yl)-2-methyl-3,4-dihydroquinolin-1(2H)-yl)ethan-1-one;
tert-butyl (S)-(4-(1-acetyl-2-methyl-1,2,3,4-tetrahydroquinolin-6-yl)benzyl)((2-(2-aminopy-rimidin-5-yl)-7-methyl-4-morpholinothieno[3,2-d]pyrimidin-6-yl)methyl)carbamate;
(S)-1-(6-(4-((((2-(2-aminopyrimidin-5-yl)-7-methyl-4-morpholinothieno[3,2-d]pyrimidin-6-yl)methyl)amino)methyl)phenyl)-2-methyl-3,4-dihydroquinolin-1(2H)-yl)ethan-1-one;
(S)-N-(4-(1-acetyl-2-methyl-1,2,3,4-tetrahydroquinolin-6-yl)benzyl)-N-((2-(2-aminopyrim-idin-5-yl)-7-methyl-4-morpholinothieno[3,2-d]pyrimidin-6-yl)methyl)acetamide;
(S)-N-(4-(1-acetyl-2-methyl-1,2,3,4-tetrahydroquinolin-6-yl)benzyl)-6-(6-amino-4-(trifluoro-methyl)pyridin-3-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(6-methoxypyridin-3-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(1H-indazol-4-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(2-(difluoromethyl)-1H-benzo[d]imidazol-1-yl)-8-morpholinoimidazo[1,2-a]pyrazine-2-carboxamide;
(S)-N-(2-(4-(1-Acetyl-2-methyl-1,2,3,4-tetrahydroquinolin-6-yl)phenyl)propan-2-yl)-6-(2-aminopyrimidin-5-yl)-8-morpholinoimidazo[1,2-a]pyrazine-2-carboxamide;
N-((S)-1-(4-((S)-1-Acetyl-2-methyl-1,2,3,4-tetrahydroquinolin-6-yl)phenyl)ethyl)-6-(2-ami-nopyrimidin-5-yl)-8-morpholinoimidazo[1,2-a]pyrazine-2-carboxamide;
N-((R)-1-(4-((S)-1-Acetyl-2-methyl-1,2,3,4-tetrahydroquinolin-6-yl)phenyl)ethyl)-6-(2-ami-nopyrimidin-5-yl)-8-morpholinoimidazo[1,2-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-8-(piperidin-1-yl)imidazo[1,2-a]pyrazine-2-carboxamide;
(S)-N-(4-(1-Acetyl-2-methyl-1,2,3,4-tetrahydroquinolin-6-yl)benzyl)-2-(2-aminopyrimidin-5-yl)-4-morpholinopyrrolo[2,1-f][1,2,4]triazine-6-carboxamide;
(S)-N-(4-(1-Acetyl-2-methyl-1,2,3,4-tetrahydroquinolin-6-yl)benzyl)-2-(2-aminopyrimidin-5-yl)-7-methyl-4-morpholinopyrrolo[2,1-f][1,2,4]triazine-6-carboxamide;
(S)-N-(4-(1-Acetyl-2-methyl-1,2,3,4-tetrahydroquinolin-6-yl)benzyl)-6-(2-aminopyrimidin-5-yl)-4-morpholinopyrazolo[1,5-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-7-chloro-4-morpholinopyrazolo[1,5-a]pyrazine-2-carboxamide;
(S)-N-(4-(1-Acetyl-2-methyl-1,2,3,4-tetrahydroquinolin-6-yl)benzyl)-3-(2-aminopyrimidin-5-yl)-1-morpholinopyrrolo[1,2-a]pyrazine-7-carboxamide;
(S)-N-(4-(1-Acetyl-2-methyl-1,2,3,4-tetrahydroquinolin-6-yl)benzyl)-7-(2-aminopyrimidin-5-yl)-5-morpholinoimidazo[1,2-c]pyrimidine-2-carboxamide;
(S)-N-(4-(1-Acetyl-2-methyl-1,2,3,4-tetrahydroquinolin-6-yl)benzyl)-2-(2-aminopyrimidin-5-yl)-9-methyl-6-morpholino-9H-purine-8-carboxamide;
(S)-N-(4-(1-Acetyl-2-methyl-1,2,3,4-tetrahydroquinolin-6-yl)benzyl)-6-(2-aminopyrimidin-5-yl)-8-morpholino-[1,2,4]triazolo[1,5-a]pyrazine-2-carboxamide;
(S)-N-(4-(1-Acetyl-2-methyl-1,2,3,4-tetrahydroquinolin-6-yl)benzyl)-6-(2-aminopyrimidin-5-yl)-8-morpholino-[1,2,4]triazolo[1,5-a]pyridine-2-carboxamide;
(S)-N-(4-(1-Acetyl-2-methyl-1,2,3,4-tetrahydroquinolin-6-yl)benzyl)-6-(2-aminopyrimidin-5-yl)-8-morpholinoimidazo[1,2-a]pyridine-2-carboxamide;
(S)-N-(4-(1-Acetyl-2-methyl-1,2,3,4-tetrahydroquinolin-6-yl)benzyl)-5-(2-aminopyrimidin-5-yl)-7-morpholinopyrazolo[1,5-a]pyrimidine-2-carboxamide;
N-(4-(1-Acetyl-2-methylindolin-5-yl)benzyl)-6-(2-aminopyrimidin-5-yl)-8-morpholinoimidazo[1,2-a]pyrazine-2-carboxamide;
N-(4-(1-Acetyl-2-methyl-4-propoxyindolin-5-yl)benzyl)-6-(2-aminopyrimidin-5-yl)-8-morpholinoimidazo[1,2-a]pyrazine-2-carboxamide; and
(S)-1-(6-(4-((((6-(2-Aminopyrimidin-5-yl)-8-morpholinoimidazo[1,2-a]pyrazin-2-yl)methyl)(hydroxy)amino)methyl)phenyl)-2-methyl-3,4-dihydroquinolin-1(2H)-yl)ethan-1-one.

30. The compound of Formula (I), salt, solvate or stereoisomer of any one of claims 1 to 29, wherein the compound is a dual inhibitor of a protein kinase enzyme and a bromodomain protein.

31. The compound of Formula (I), salt, solvate or stereoisomer of any one of claims 1 to 30, wherein the compound is a dual inhibitor of PI3K and BRD4.

32. A pharmaceutical composition comprising a compound of Formula (I), salt, solvate or stereoisomer of any one of claims 1 to 31 and a pharmaceutically acceptable excipient.

33. A method of inhibiting a protein kinase enzyme and a bromodomain protein, comprising contacting a compound of Formula (I), salt, solvate or stereoisomer of any one of claims 1 to 31, or the pharmaceutical composition of claim 32, with a protein kinase enzyme and a bromodomain protein.

34. The compound of Formula (I), salt, solvate or stereoisomer of any one of claims 1 to 31, or the pharmaceutical composition of claim 32, for use in the prevention or treatment of cancer.

35. The compound of Formula (I), salt, solvate or stereoisomer of any one of claims 1 to 31, or the pharmaceutical composition of claim 32, for use in the prevention or treatment of a Myc-dependent cancer.

36. The compound of Formula (I), salt, solvate or stereoisomer of any one of claims 1 to 31, or the pharmaceutical composition of claim 32, for use in the prevention or treatment of a Myc-dependent cancer selected from the group consisting of lymphoma, acute myeloid leukaemia, multiple myeloma, neuroblastoma, and medulloblastoma.

37. A method of preventing or treating cancer in a subject, comprising administering an effective amount of the compound of Formula (I), salt, solvate or stereoisomer of any one of claims 1 to 31, or the pharmaceutical composition of claim 32, to the subject.

38. Use of a compound of Formula (I), salt, solvate or stereoisomer of any one of claims 1 to 31, or of the pharmaceutical composition of claim 32, in the manufacture of a medicament for use in preventing or treating a cancer.

39. Use of a compound of Formula (I), salt, solvate or stereoisomer of any one of claims 1 to 31, or of the pharmaceutical composition of claim 32, in the manufacture of a medicament for use in preventing or treating a Myc-dependent cancer in a subject.

40. The use of claim 39, wherein the Myc-dependent cancer is selected from the group consisting of lymphoma, acute myeloid leukaemia, multiple myeloma, neuroblastoma, and medulloblastoma.
